(19) United States
(12) Patent Application Publication
(10) Pub. No.: US 2026/0014261 A1
(43) Pub. Date: Jan. 15, 2026

(54) TARGETED PROTEIN MODIFICATION

(71) Applicant: Weatherwax Biotechnologies Corporation, San Francisco, CA (US)

(72) Inventors: Armand B. COGNETTA, III, San Francisco, CA (US); Corentine M.C. LAURIN, Kufstein (AT); Veronika M. SHOBA, San Francisco, CA (US); Rodrigo RODRIGUEZ, San Diego, CA (US)

(21) Appl. No.: 19/213,795

(22) Filed: May 20, 2025

Related U.S. Application Data
(63) Continuation of application No. PCT/US2023/080384, filed on Nov. 17, 2023.
(60) Provisional application No. 63/427,164, filed on Nov. 22, 2022.

(51) Int. Cl. A61K 47/55; A61K 47/54

(52) U.S. Cl. CPC A61K 47/55; A61K 47/54; A61K 47/545

(57) ABSTRACT

Provided are compounds that may bind a target protein, and result in modification of the target protein. The compounds may further bind a modifier protein. The modifier protein may carry out or enhance the modification of the target protein. The modification may activate or reactivate the target protein. Also provided are methods of using the compounds.

### CROSS-REFERENCE TO RELATED APPLICATIONS

This application is a continuation of International Application No. PCT/US2023/080384 filed Nov. 17, 2023, which claims the benefit of U.S. Provisional Patent Application No. 63/427,164 filed Nov. 22, 2022, all of which are hereby incorporated by reference in their entireties.

### SEQUENCE LISTING

The instant application contains a Sequence Listing which has been submitted electronically in XML format and is hereby incorporated by reference in its entirety. Said XML copy, created on Sep. 8, 2025, is named 57739-701-301_SL.xml and is 2,285 bytes in size.

### BACKGROUND

A need exists for compounds that, when used, result in the modification of a protein. A need exists in the medicinal arts for selective modification of target proteins.

### SUMMARY

Provided herein are modifier proteins targeting chimeric (OmniTAC) compounds comprising a targeting ligand, a recruiting ligand, and a linker; wherein the targeting ligand is attached to the recruiting ligand via the linker; wherein the targeting ligand is configured to bind to a target protein, and the recruiting ligand is configured to bind to a modifier protein such that the modifier protein induces a change to the target protein, or wherein the recruiting ligand is configured to bind to a nucleic acid such that the target protein is targeted to the nucleic acid; and wherein the modifier protein comprises a protein that induces activation, stabilization, or corrects misfolding of the target protein.

Provided herein are in vivo engineered proteins comprising: a target protein directly bound to the compound described herein.

Provided herein are methods for inducing a change in a target protein comprising contacting the target protein with a modifier protein via the compounds described herein such that the modifier protein induces a change to the target protein.

### BRIEF DESCRIPTION OF THE DRAWINGS

FIG. 1 shows a bifunctional molecule (BFM). The BFM is a heterobifunctional molecule comprising a targeting ligand and recruiting ligand bound by a linker. The BFM connects a target protein/drug target to a modifier protein/cellular enzyme. When target and modifier proteins are brought together, an induced change in post-translational modification repairs the target.

FIG. 2A shows an example of a p53 targeting ligand where p53 is a target protein. The compound in the figure is referred to as SCH 529074, is an example of a pharmacochaperone, and has a binding affinity (Kd) for p53 of about 1 μM. The ligand may also be used as a recruiting ligand to bind p53 as a modifier protein.

FIG. 2B shows a structure of a ligand that may be used to bind p53 ligand and a structure of a molecule coupled to a linker that may be connected to a recruiting ligand. Different linker attachment points or linkers may be used.

FIG. 3A shows an example of a VHL-based VHL targeting ligand where VHL is included as a target protein. The compound in the figure is referred to as VHL ligand 1, is an example of a pharmacochaperone, and has a binding affinity (Kd) for VHL of about 50 nM. The ligand may also be used as a recruiting ligand to bind VHL as a modifier protein.

FIG. 3B shows a structure of a ligand that may be used to bind a VHL protein and a structure of a molecule coupled to a linker that may be connected to a recruiting ligand. Different linker attachment points or linkers may be used.

FIG. 4 shows Western blot data showing protein induction of p21 and MDM2 through p53 mutant activation in H1299 cells using the bifunctional molecules WXB641, WXB661 and WXB671.

FIG. 5 shows quantification of Western blot showing protein induction of p21 through p53 mutant activation in Huh7 cells using the bifunctional molecules WXB641, WXB661 and WXB671. Huh7 cells (endogenous p53) were treated with DMSO, control compounds or BFMs (1 or 0.2 μM) for 24 h.

FIG. 6 shows a dose response curve showing activation of p53 mutants for two bifunctional molecules WXB661 and WXB672.

FIG. 7 shows cell viability assays showing bifunctional molecule induced cell death.

FIG. 8 shows activation of mutant GBA using bifunctional molecules that outperform clinical candidate IFG.

FIG. 9 shows a non-limiting list of modifier proteins and references for associated ligands that can be used to design recruiting ligands, organized by mechanism of action.

FIG. 10 shows a non-limiting list of modifier proteins and references for associated ligands that can be used to design recruiting ligands, organized by mechanism of action.

FIG. 11 shows cell viability assays showing bifunctional molecule induced cell death.

FIG. 12 shows a table summary of mutant p53 activation through the treatment of OmniTAC compounds compared to controls. Compounds with SC150 value less than or equal to 1 μM were deemed to be highly active (++++), compounds with SC150 between 1 and 10 μM were deemed to be very active (+++), compounds with SC150 between 10 and 20 μM were deemed to be active (++), compounds with SC150 above or equal to 20 μM were deemed to be weakly active (+).

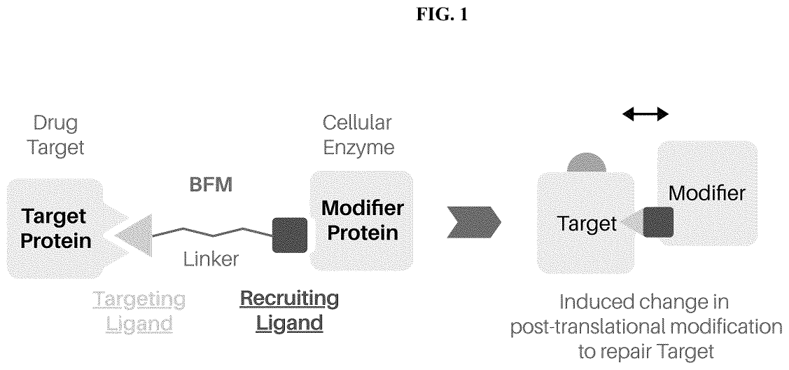

### DETAILED DESCRIPTION

Disclosed herein is a platform which may be useful for activating or reactivating a target protein. The platform may include compounds such as modifier protein targeting chimeric (OmniTAC) compounds, and methods of using them.

Compounds of the present disclosure are capable of providing selective, targeted protein modifications. Current drug approaches to therapeutics generally involve using small molecules, biologics, and/or genetic therapies to modify a biological target of interest, e.g., a protein target. Such approaches are limited in that small molecules generally have a small range of method of action and may not be catalytic; and biologic or genetic therapies may have poor oral bioavailability and are difficult to target intracellular targets. The compounds of the present disclosure advantageously bring a target protein in proximity with a modifier protein, which provides a modification to the target protein to exert a therapeutic effect. This approach leverages the protein modification capabilities of existing modifier proteins to induce changes in disease-associated target proteins in vivo, enabling the development of highly specific and potent drugs.

### OmniTAC Compounds

Provided herein, in some embodiments, are compounds and pharmaceutical compositions comprising said compounds. In some embodiments a compound described herein comprises a targeting ligand, a linker, or a recruiting ligand. The compound may comprise or consist of a modifier protein targeting chimeric (OmniTAC) compound.

Disclosed herein, in some embodiments, are OmniTAC compounds. The OmniTAC compound may comprise a heterobifunctional compound (BFM). The OmniTAC compound may include a targeting ligand. The OmniTAC compound may include a recruiting ligand. The OmniTAC compound may include a linker. The linker may attach the targeting ligand to the recruiting ligand. Some embodiments include a modifier protein targeting chimeric (OmniTAC) compound comprising a targeting ligand, a recruiting ligand, and a linker; wherein the targeting ligand is attached to the recruiting ligand via the linker.

In some embodiments, the compound has a structure of Formula I:

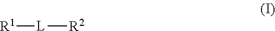

wherein R¹ is the targeting ligand; L is the linker; and R² is the recruiting ligand. As used throughout the present disclosure, “R¹”, “targeting ligand”, and “TL” are used interchangeably; “L” and “linker” are used interchangeably; and “R²”, “recruiting ligand”, and “RL” are used interchangeably.

In some embodiments, the compound includes some aspect of a natural or organic compound. In some embodiments, the compound is synthetic. In some embodiments, the compound is engineered. The compound may be purified. In some embodiments, the compound is substantially pure. As used herein, “substantially pure” means that the compound is substantially separated or isolated from contaminants or impurities. In some embodiments, a substantially pure compound comprises less than 10%, less than 5%, less than 2%, less than 1%, less than 0.5%, or less than 0.1% by weight of impurities.

In some embodiments, the compound comprises or consists of a small molecule. An example of a small molecule includes an organic compound having a molecular weight of less than 900 daltons. The compound may have a molecular weight below 2500 daltons, below 2250 daltons, below 2000 daltons, below 1750 daltons, below 1500 daltons, or below 1250 daltons. The compound may have a molecular weight below 1000 daltons, below 900 daltons, below 800 daltons, below 700 daltons, below 600 daltons, or below 500 daltons. The compound may have a molecular weight greater than 2500 daltons, greater than 2250 daltons, greater than 2000 daltons, greater than 1750 daltons, greater than 1500 daltons, or greater than 1250 daltons. The compound may have a molecular weight greater than 1000 daltons, greater than 900 daltons, greater than 800 daltons, greater than 700 daltons, greater than 600 daltons, or greater than 500 daltons.

The compound may be included in a pharmaceutical composition. The compound may be administered to a subject. The compound may be included in a method described herein. For example, the compound may be used in a protein modification method or in a treatment method. Some embodiments include a method of making a compound disclosed herein.

### Targeting Ligands and Target Proteins

Disclosed herein, in some embodiments, are targeting ligands. The targeting ligand may include a ligand of a protein such as a target protein. The targeting ligand may be configured to bind to a target protein. The targeting ligand may bind the target protein. The targeting ligand may include a moiety that binds the target protein. The targeting ligand may be a part of a heterobifunctional compound such as an OmniTAC compound. The targeting ligand may connect to a linker or recruiting ligand. In some embodiments, the targeting ligand is incorporated into an in vivo engineered protein.

The targeting ligand may include a small molecule. In some embodiments, the targeting ligand comprises a small molecule moiety. In some embodiments, the targeting ligand has a molecular weight of 200, 300, 400, 500, 600, 700, 800, 900, 1000, 1250, 1500, 1750, 2000, 2250, or 2500 daltons, or a range defined by any two of the aforementioned numbers of daltons.

Disclosed herein, in some embodiments, are targeting ligands that bind a target protein. The targeting ligand may bind directly to the target protein. In some embodiments, the binding between the targeting ligand and the target protein is covalent. The covalent binding may be with a cysteine, lysine, or methionine, or any other reactive residue of the target protein. In some embodiments, the binding between the targeting ligand and the target protein is non-covalent.

Electrophiles reactive to lysines, cysteines and methionines can be inserted between the ligand and the linker, or independently attached to the recruiting ligand (RL), or the targeting ligand (TL), or to the linker. Examples of the target ligand or recruiting ligand binding with cysteine, lysine, or methionine are shown below (R=TL or RL, attachment point shows linker-TL or linker-RL):

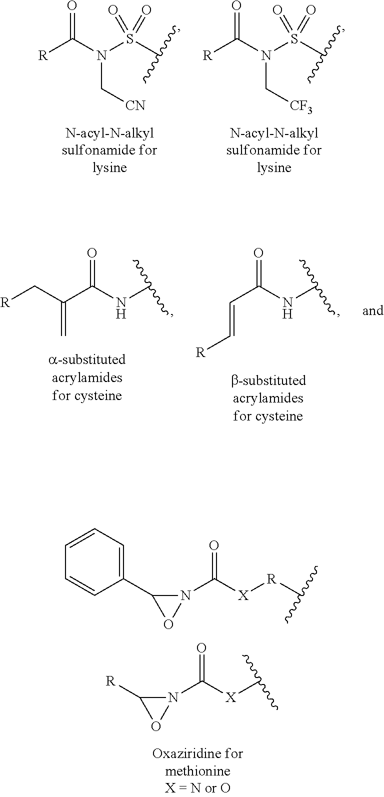

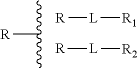

Non-limiting, exemplary cysteine reactive groups that can be attached to the RL or the TL are shown below:

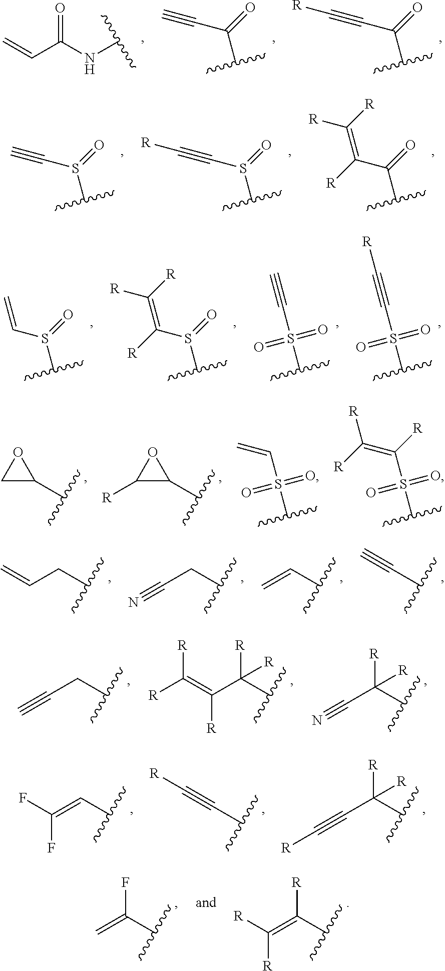

In some embodiments, the binding between the target protein and the targeting ligand comprises a binding affinity with an equilibrium dissociation constant (Kd) below 100 μM, a Kd below 90 μM, a Kd below 80 μM, a Kd below 70 μM, a Kd below 60 μM, a Kd below 50 μM, a Kd below 45 μM, a Kd below 40 μM, a Kd below 35 μM, a Kd below 30 μM, a Kd below 25 μM, a Kd below 20 μM, a Kd below 15 μM, a Kd below 14 μM, a Kd below 13 μM, a Kd below 12 μM, a Kd below 11 μM, a Kd below 10 μM, a Kd below 9 μM, a Kd below 8 μM, a Kd below 7 μM, a Kd below 6 μM, a Kd below 5 μM, a Kd below 4 μM, a Kd below 3 μM, a Kd below 2 μM, or a Kd below 1 μM. In some embodiments, the binding between the target protein and the targeting ligand comprises a binding affinity with an equilibrium dissociation constant (Kd) of at least 100 μM, a Kd of at least 90 μM, a Kd of at least 80 μM, a Kd of at least 70 μM, a Kd of at least 60 μM, a Kd of at least 50 μM, a Kd of at least 45 μM, a Kd of at least 40 μM, a Kd of at least 35 μM, a Kd of at least 30 μM, a Kd of at least 25 μM, a Kd of at least 20 μM, a Kd of at least 15 μM, a Kd of at least 14 μM, a Kd of at least 13 μM, a Kd of at least 12 μM, a Kd of at least 11 μM, a Kd of at least 10 μM, a Kd of at least 9 μM, a Kd of at least 8 μM, a Kd of at least 7 μM, a Kd of at least 6 μM, a Kd of at least s5 μM, a Kd of at least 4 μM, a Kd of at least 3 μM, a Kd of at least 2 μM, or a Kd of at least 1 μM.

Disclosed herein, in some embodiments are targeting ligands with the following structure:

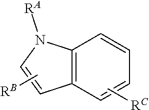

wherein:
R^{A} is a C₁-C₅ alkyl or cycloalkyl, such as the non-limiting example shown below:

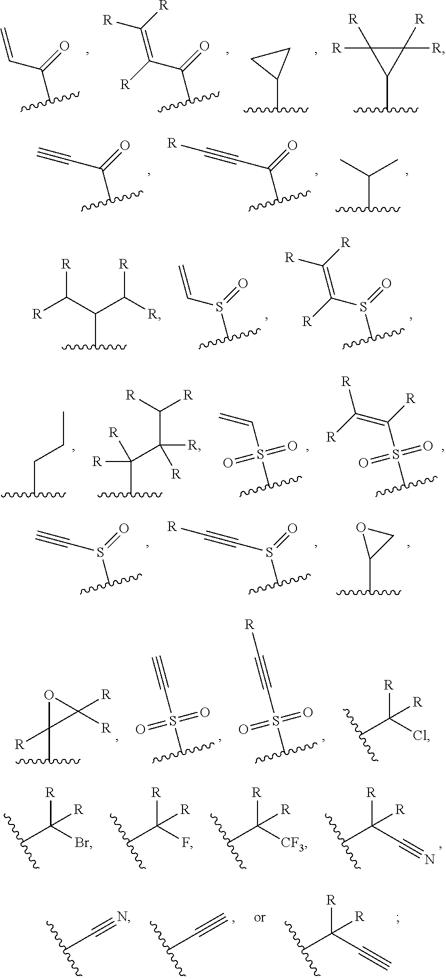

R^{B} comprises aromatic or heteroaromatic groups, such as the non-limiting examples shown below:

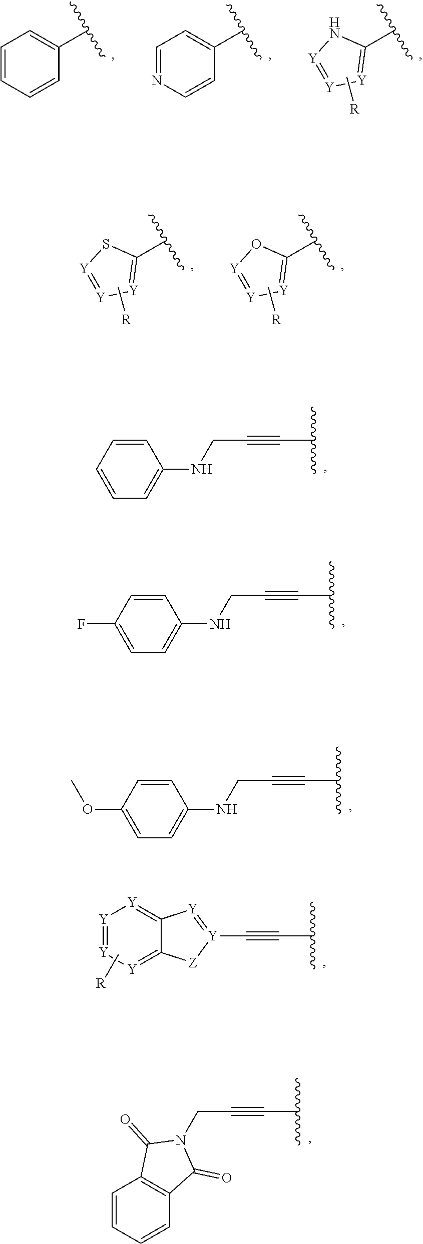

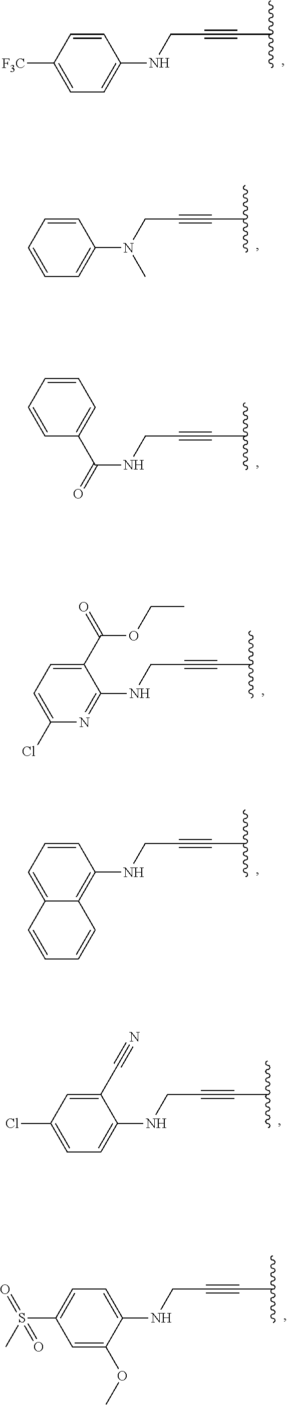

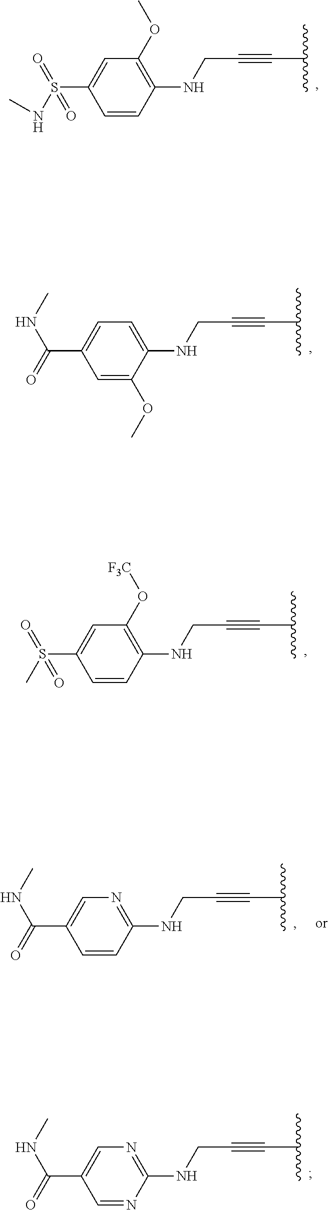 [SMILES: *C#CCNc1ccc(S(=O)(=O)NC)cc1OC ; *C#CCNc1ccc(C(=O)NC)cc1OC ; *C#CCNc1ccc(S(C)(=O)=O)cc1OC(F)(F)F ; *C#CCNc1ccc(C(=O)NC)cn1 ; *C#CCNc1ncc(C(=O)NC)cn1]

and
R^{C} is

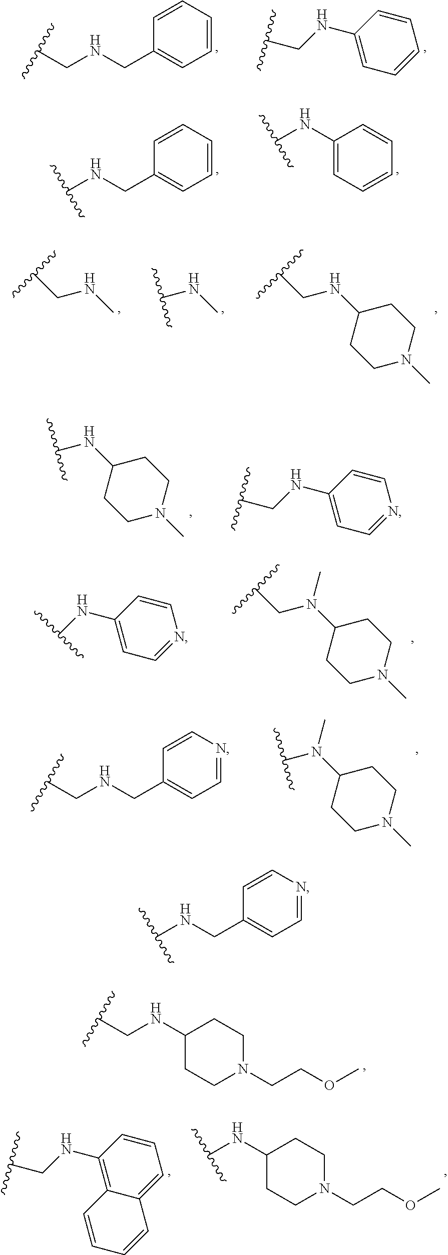

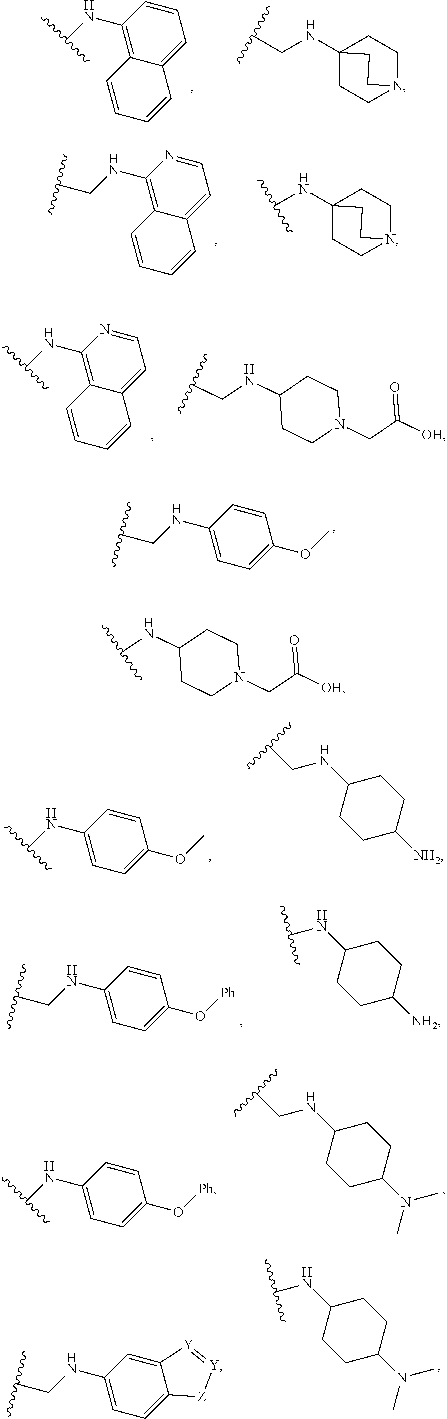

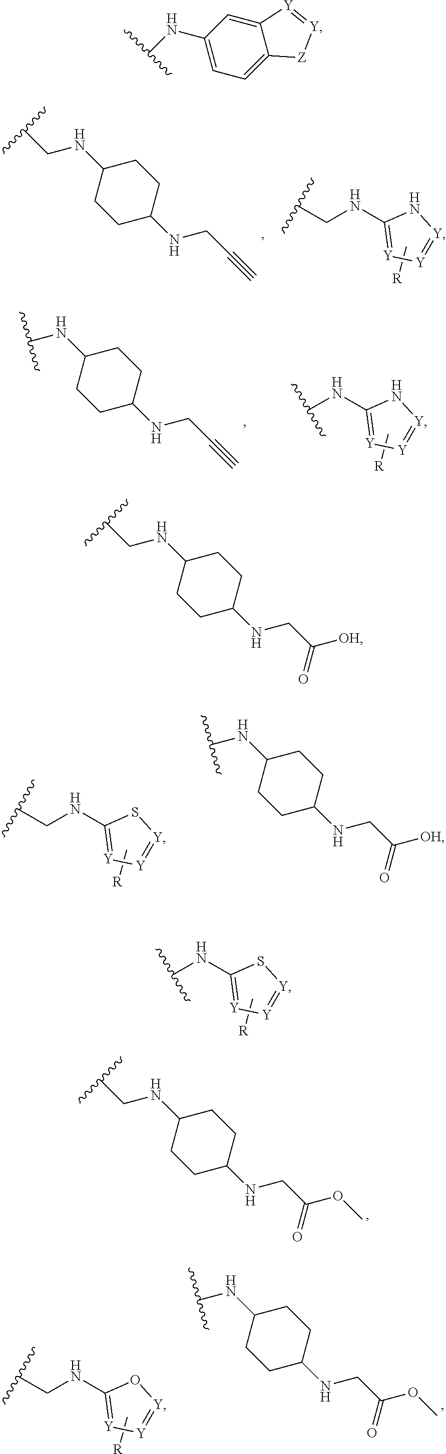

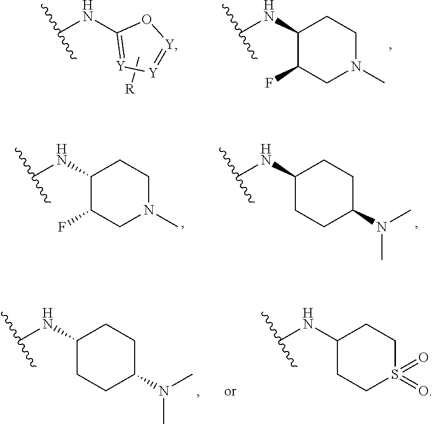

The R groups in R^{A}, R^{B}, and R^{C} described above can be —CN, halogens, alkyls, alkoxys, or alkylamines. The targeting ligand can comprise any combination of R^{A}, R^{B}, and R^{C} as described herein.

Disclosed herein, in some embodiments, are targeting ligand with the following structure:

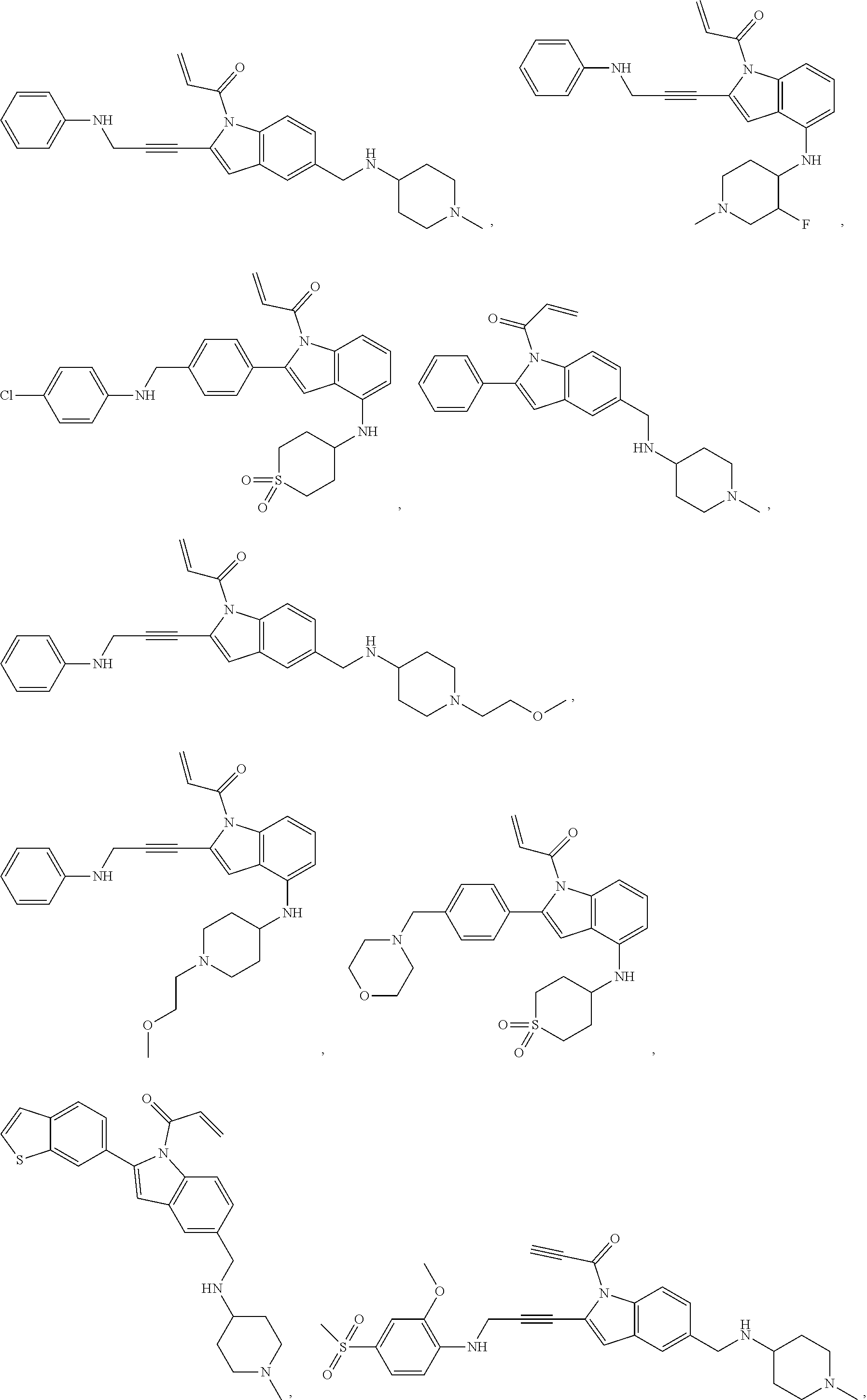

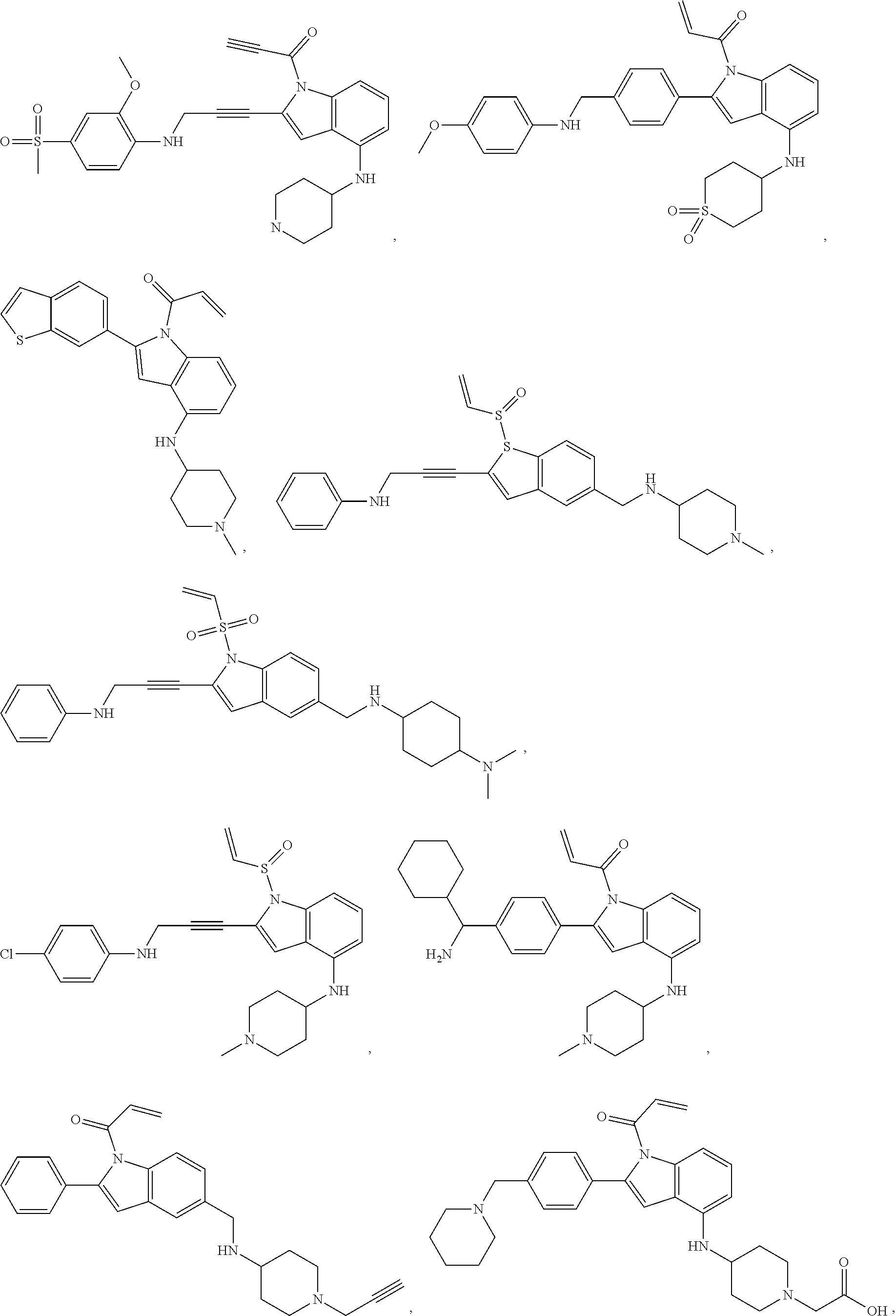

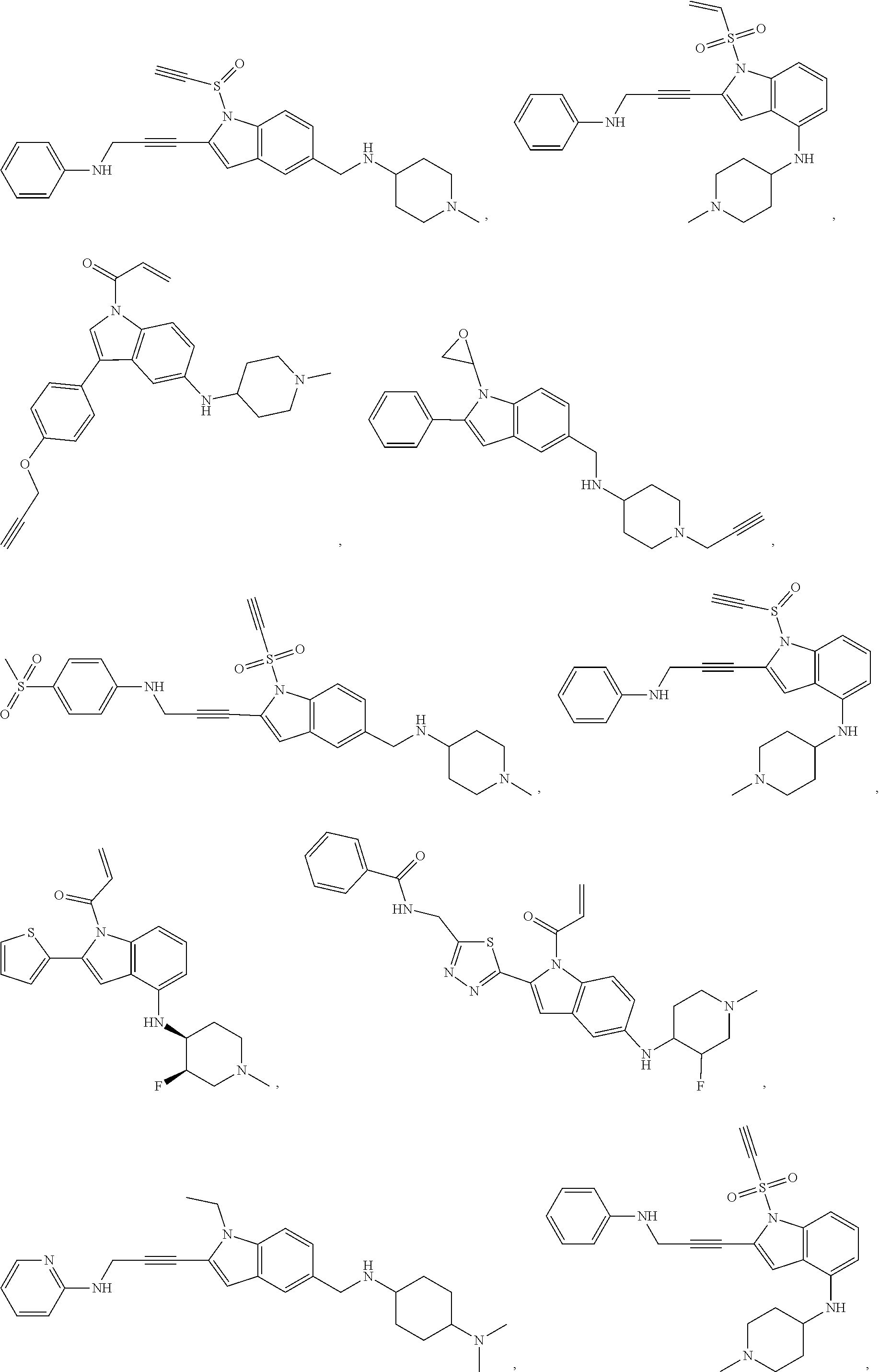

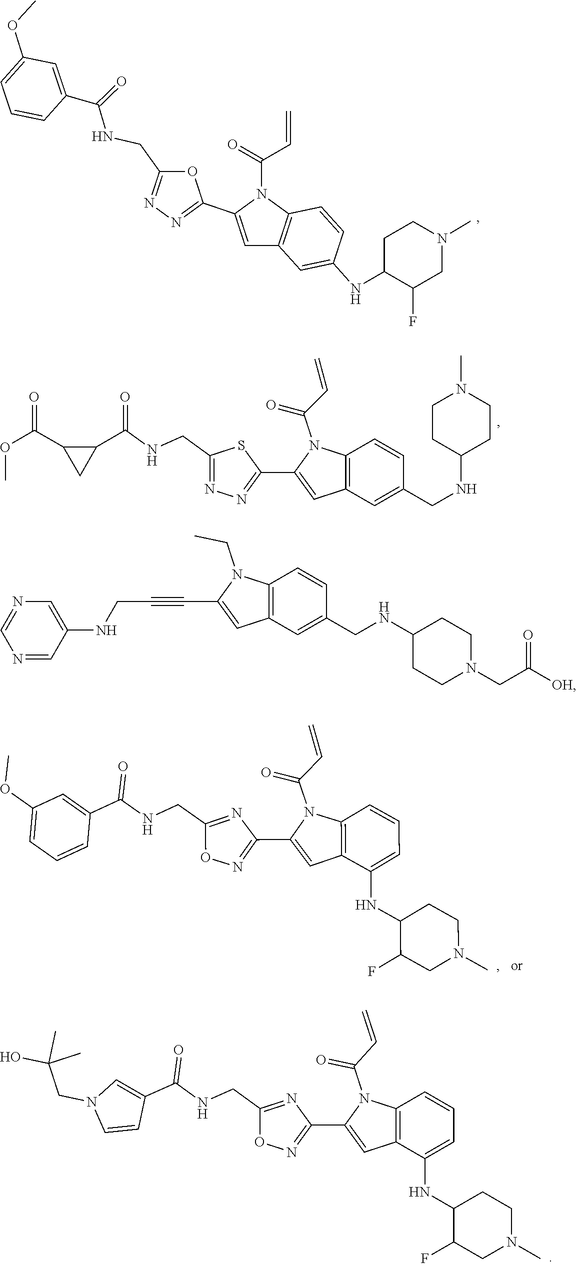

Disclosed herein, in some embodiments, is a scaffold consisting of a targeting ligand and a linker with the following structure:

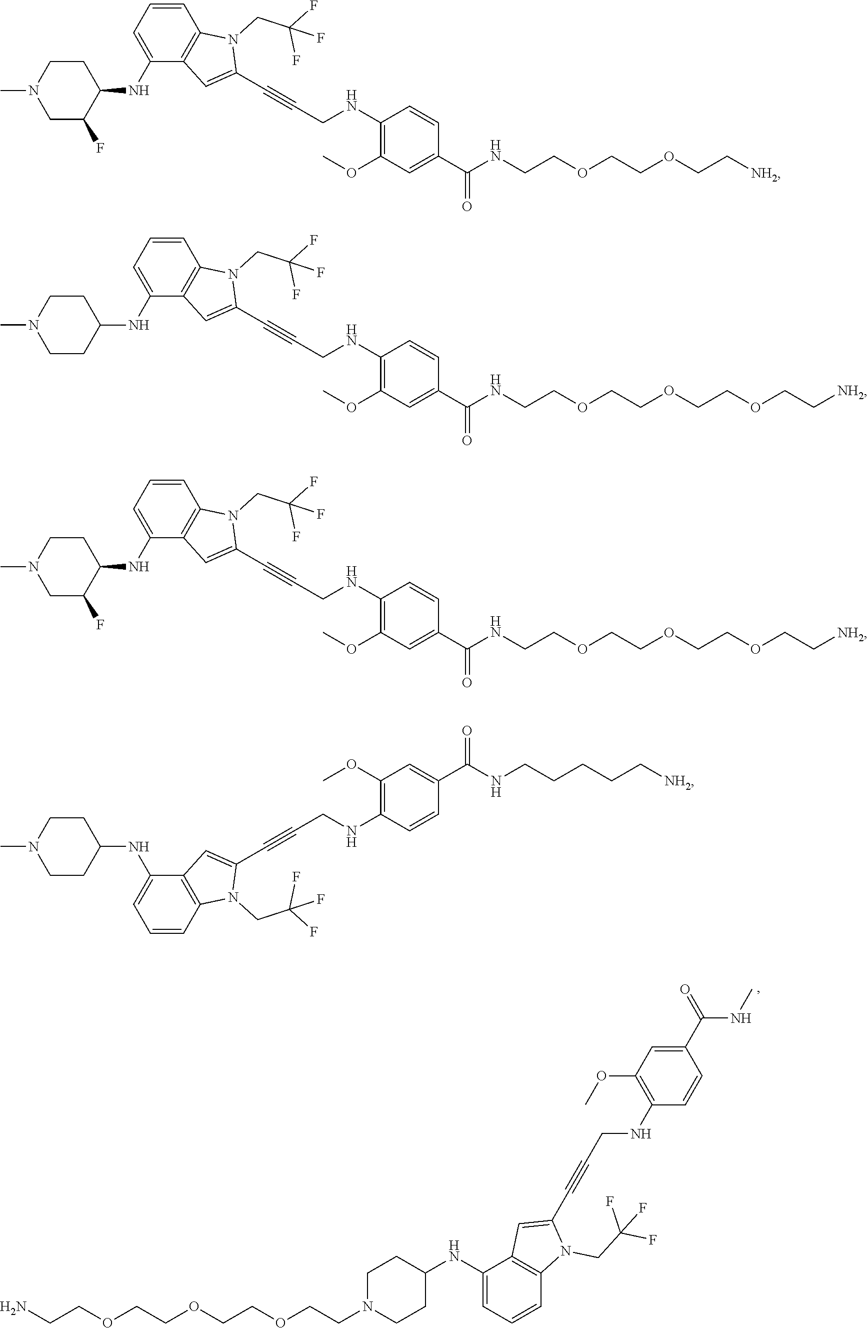 [SMILES: COc1cc(C(=O)NCCOCCOCCN)ccc1NCC#Cc1cc2c(N[C@@H]3CCN(C)C[C@@H]3F)cccc2n1CC(F)(F)F ; COc1cc(C(=O)NCCOCCOCCOCCN)ccc1NCC#Cc1cc2c(NC3CCN(C)CC3)cccc2n1CC(F)(F)F ; COc1cc(C(=O)NCCOCCOCCOCCN)ccc1NCC#Cc1cc2c(N[C@@H]3CCN(C)C[C@@H]3F)cccc2n1CC(F)(F)F ; COc1cc(C(=O)NCCCCCN)ccc1NCC#Cc1cc2c(NC3CCN(C)CC3)cccc2n1CC(F)(F)F ; CNC(=O)c1ccc(NCC#Cc2cc3c(NC4CCN(CCOCCOCCOCCN)CC4)cccc3n2CC(F)(F)F)c(OC)c1]

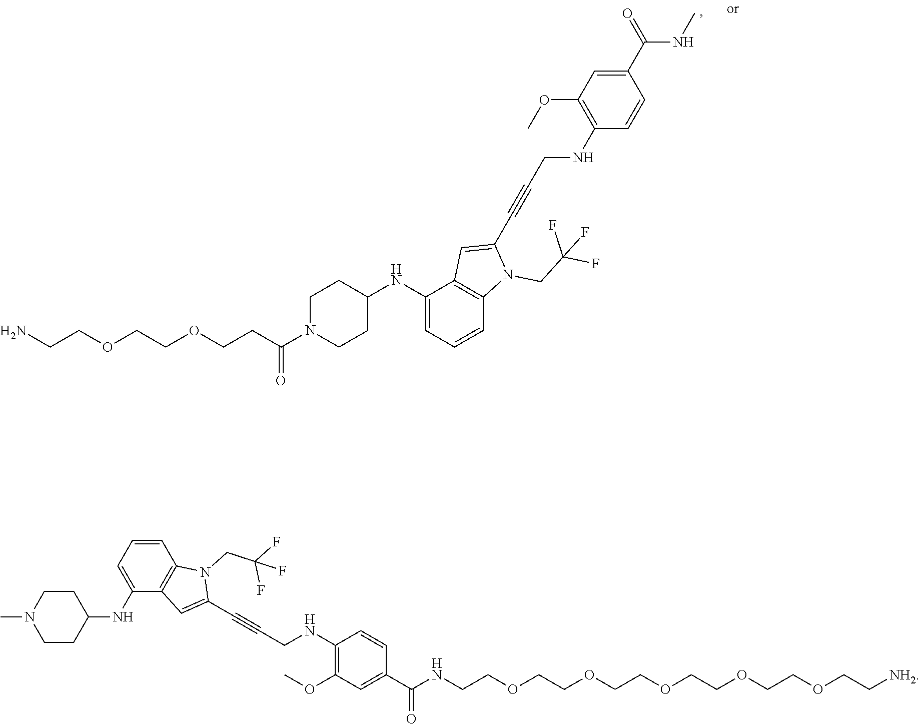

Disclosed herein, in some embodiments, is a scaffold consisting of a targeting ligand and a linker with the following structure:

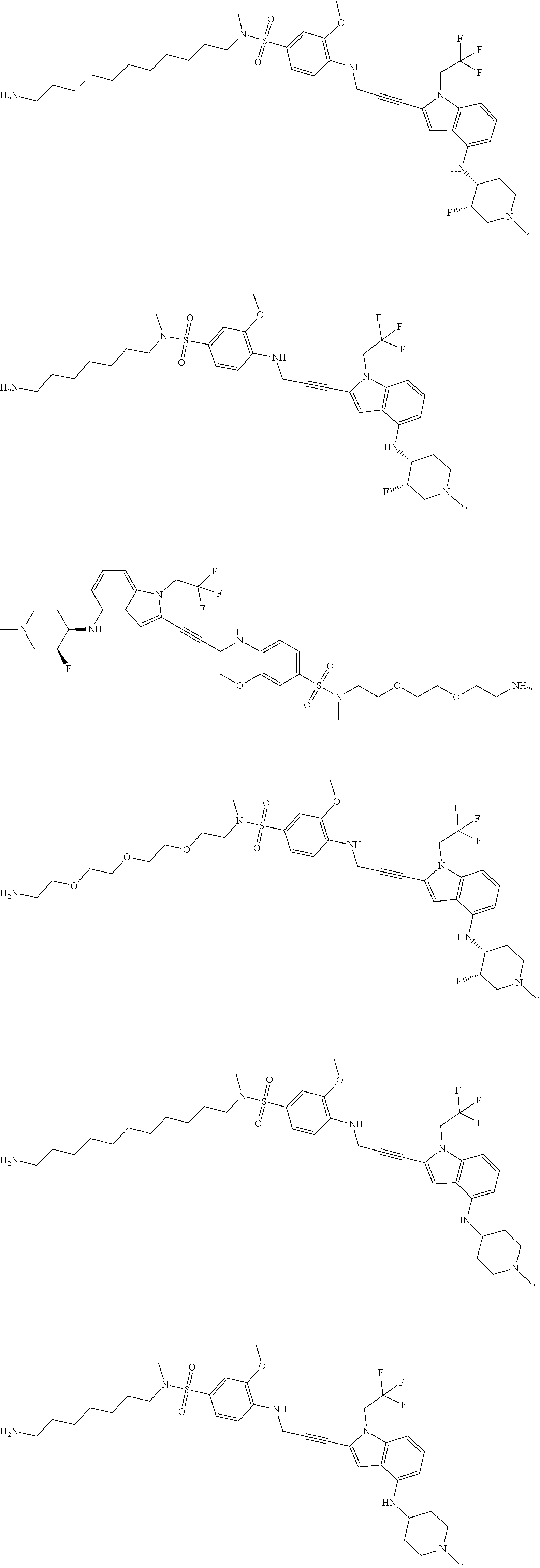

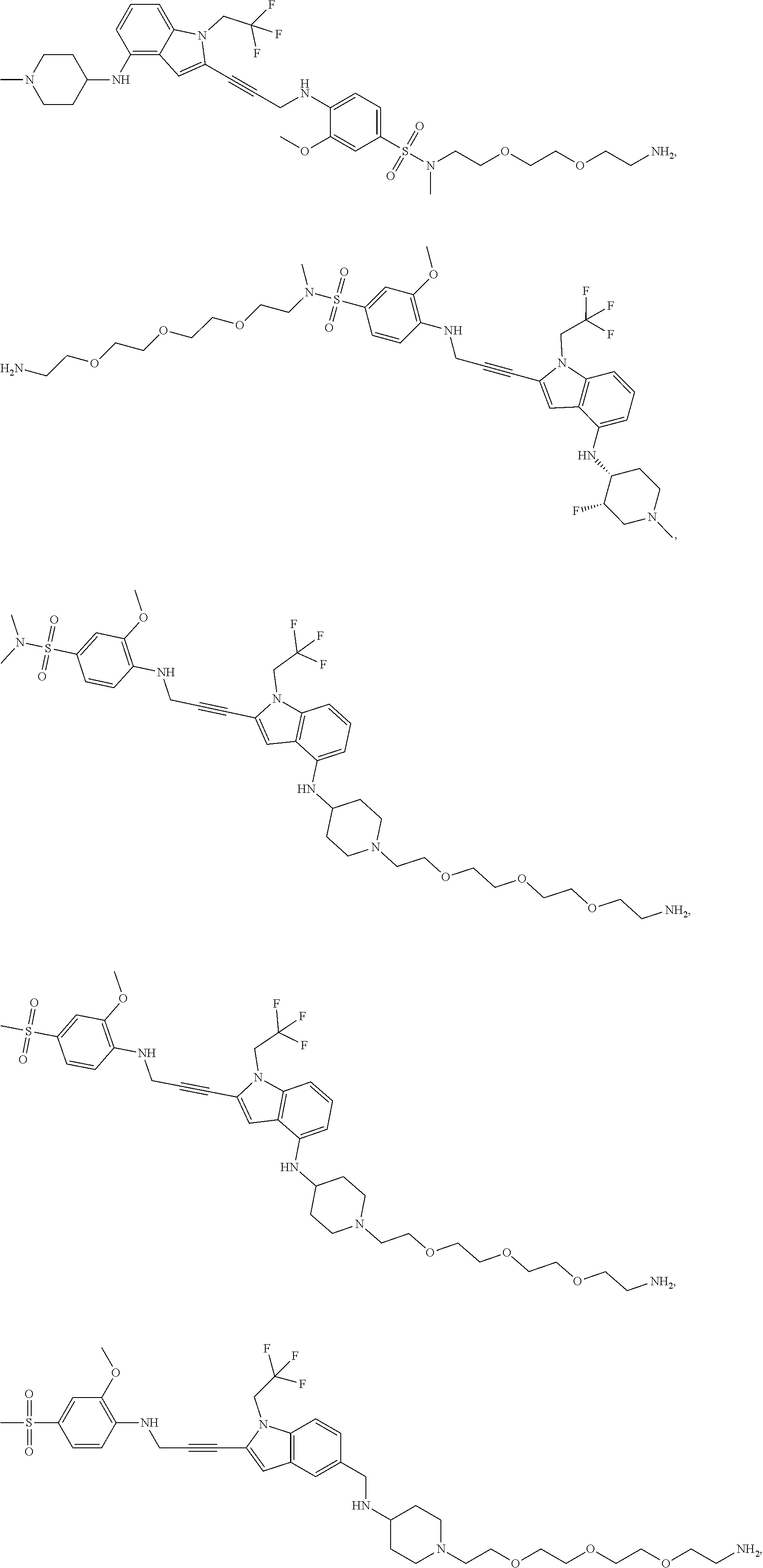

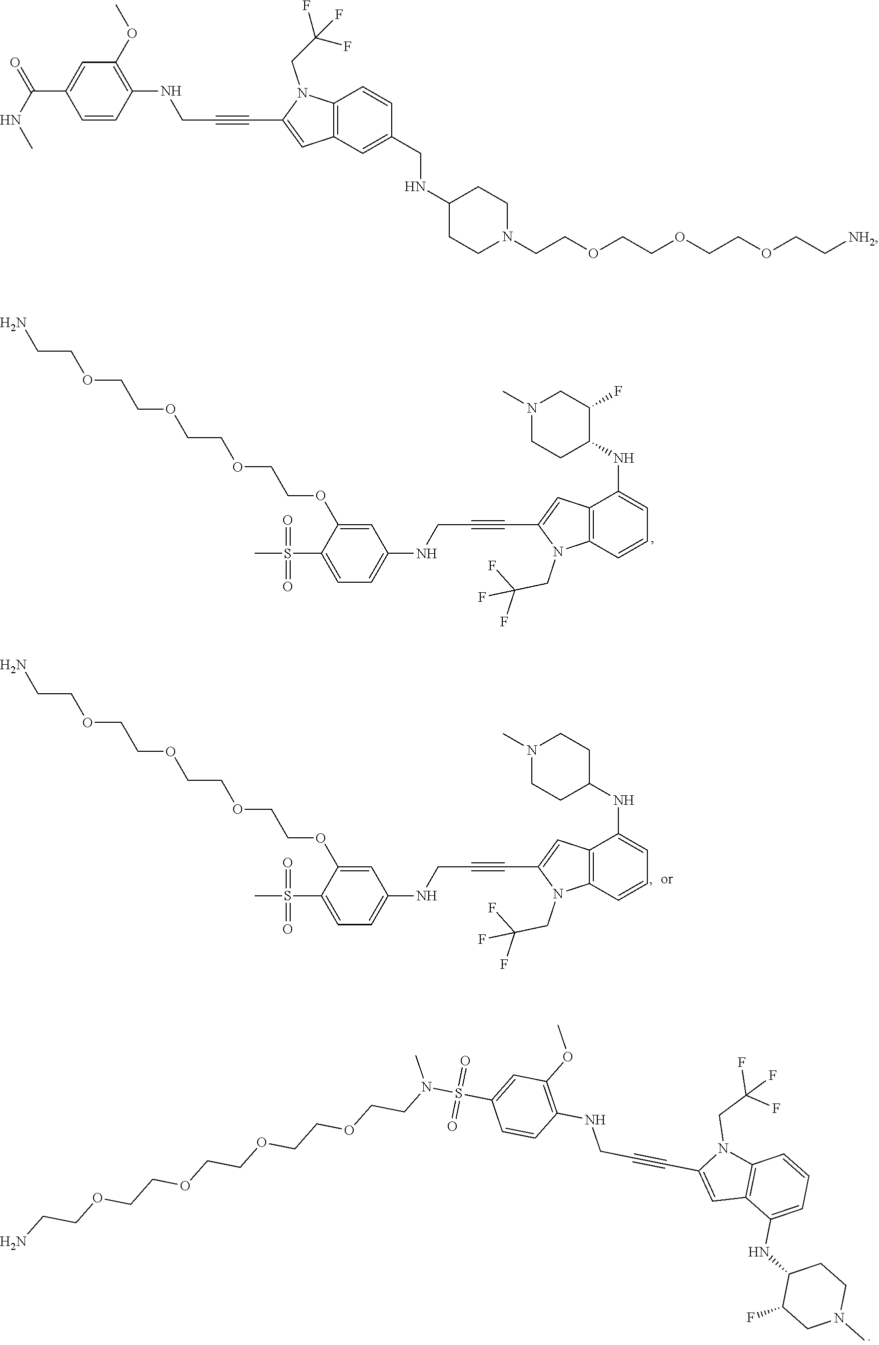 [SMILES: CNC(=O)c1ccc(NCC#Cc2cc3cc(CNC4CCN(CCOCCOCCOCCN)CC4)ccc3n2CC(F)(F)F)c(OC)c1 ; CN1CC[C@@H](Nc2cccc3c2cc(C#CCNc2ccc(S(C)(=O)=O)c(OCCOCCOCCOCCN)c2)n3CC(F)(F)F)[C@@H](F)C1 ; CN1CCC(Nc2cccc3c2cc(C#CCNc2ccc(S(C)(=O)=O)c(OCCOCCOCCOCCN)c2)n3CC(F)(F)F)CC1 ; COc1cc(S(=O)(=O)N(C)CCOCCOCCOCCOCCN)ccc1NCC#Cc1cc2c(N[C@@H]3CCN(C)C[C@@H]3F)cccc2n1CC(F)(F)F]

In some embodiments, the linker connects to the targeting ligand at the aromatic ring of the RB substituent, or to the aliphatic amine RC substituent. In some embodiments, the linker connects to the targeting ligand at the phenyl ring. In some embodiments, the linker connects to the targeting ligand at the piperidinyl ring. In some embodiments, the linker connects via an ether, a thioether, an amide, an ester, a carbamate, a sulfone, a sulfonamide, a triazole or a urea.

Disclosed herein, in some embodiments, are target proteins. A target protein may include a protein intended to be modified, e.g. by a modifier protein. The target protein may be an enzymatic interacting partner for the modifier protein. The target protein may be modified by the modifier protein. The target protein may be any protein upon which the modifier protein exerts its effects. In some embodiments, the modifier protein may be modified by the target protein.

Examples of target proteins may include, in non-limiting examples, structural proteins, signaling proteins such as receptor proteins, channel proteins, or enzymes. In some embodiments, the target protein comprises a tumor suppressor, metabolic enzyme, protein aggregate, or haploinsufficient protein. The target protein may include a tumor suppressor. The target protein may be a metabolic protein. The target protein may include an enzyme. The target protein may include an aggregate protein. The target protein may include a haploinsufficient protein.

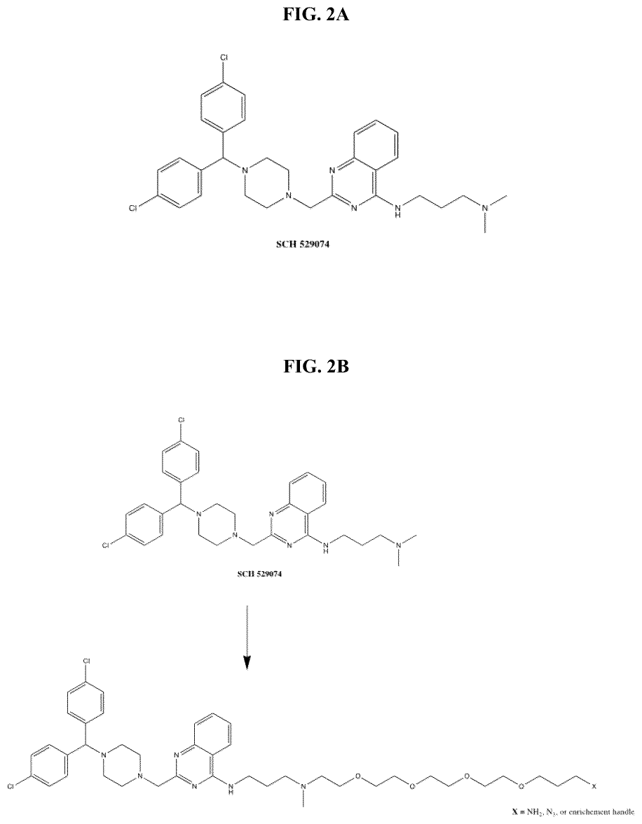

In some embodiments, the target protein includes a tumor suppressor. An example of a tumor suppressor includes tumor protein P53 (P53 or p53). P53 is also an example of protein that can activate DNA repair, arrest growth, or initiate apoptosis. P53 is controlled by a multitude of post-translational modifications (PTMs) such as phosphorylation. Phosphorylation of P53 may dictate its activation status. The target protein may include P53 or another protein that modulates DNA repair, cell growth, or apoptosis. P53 is an example of a transcription factor. A mutation in p53 is observed in over 50% of all cancers. 90% of these mutations are missense and result in inactive or dominant forms of p53. P53 has generally been an undruggable target. Previous approaches, mostly small molecules, have had only limited success in rescuing wild-type p53 function. Compared to existing pharmacochaperones, BFMs that recruit proteins capable of modulating p53 activity have a better chance of effectively re-activating p53. An example of a targeting ligand that binds to p53 is included in FIG. 2A. FIG. 2B includes an example of a targeting ligand that binds p53 and is attached to a linker. The linker may be further attached to a recruiting ligand, for example, via click or amide coupling. In some embodiments, the compound provided herein targets p53 for modification by a modifier protein described herein. In some embodiments, the modification comprises a post-translation modification of p53. Further modifications are provided herein.

The p53 may include any detail as described at uniprot.org under accession number P04637 (as last accessed Nov. 10, 2022). p53 may include a polypeptide having the following amino acid sequence: MEEPQSDPSVEPPLSQETFSDLWKLLPENNVLSPLPSQAMDDLMLSPDDIEQWFTEDPGPDEA PRMPEAAPPVAPAPAAPTPAAPAPAPSWPLSSSVPSQKTYQGSYGFRLGFLHSGTAKSVTCTY SPALNKMFCQLAKTCPVQLWVDSTPPPGTRVRAMAIYKQSQHMTEVVRRCPHHERCSDSDG LAPPQHLIRVEGNLRVEYLDDRNTFRHSVVVPYEPPEVGSDCTTIHYNYMCNSSCMGGMNR RPILTIITLEDSSGNLLGRNSFEVRVCACPGRDRRTEEENLRKKGEPHHELPPGSTKRALPNNT SSSPQPKKKPLDGEYFTLQIRGRERFEMFRELNEALELKDAQAGKEPGGSRAHSSHLKSKKG QSTSRHKKLMFKTEGPDSD (SEQ ID NO: 1). Unless otherwise specified, all references herein to amino acid residues numbering of p53 is relative to SEQ ID NO: 1. In some embodiments, the p53 of SEQ ID NO:1 is wild-type p53. The p53 may include a mutant form of p53. The mutant form may be with regard to SEQ ID NO: 1. The mutant form may include a functional fragment or SEQ ID NO: 1. In some embodiments, a functional fragment of SEQ ID NO:1 has at least some of the function of wild type p53. The mutant form of p53 may include an amino acid sequence at least 70% identical, at least 75% identical, at least 80% identical, at least 85% identical, at least 90% identical, at least 91% identical, at least 92% identical, at least 93% identical, at least 94% identical, at least 95% identical, at least 96% identical, at least 97% identical, at least 98% identical, or at least 99% identical, to SEQ ID NO: 1. The mutant form of p53 may in some instances include an amino acid sequence less than 75% identical, less than 80% identical, less than 85% identical, less than 90% identical, less than 91% identical, less than 92% identical, less than 93% identical, less than 94% identical, less than 95% identical, less than 96% identical, less than 97% identical, less than 98% identical, less than 99% identical, or less than 100% identical, to SEQ ID NO: 1.

The p53 may comprise a mutant p53. The targeting ligand may bind the p53 mutant. Some aspects relate to a heterobifunctional compound comprising: a p53 binding moiety attached to a recruiting ligand that binds a modifier protein, wherein the p53 binding moiety binds to one of a plurality of mutant forms of p53. Some aspects relate to a heterobifunctional compound comprising: a p53 binding moiety attached to a recruiting ligand that binds a modifier protein, wherein the p53 binding moiety binds a mutant form of p53. In some embodiments, the mutant form of p53 is the p53 Y220C mutant, wherein the tyrosine at amino acid position 220 of p53 is substituted with a cysteine. Some aspects include a method of activating a p53 mutant protein, comprising contacting the p53 mutant protein with a compound disclosed herein.

In some embodiments, p53 binding ligands can bind to p53's DNA binding domain (amino acids 92-312) of wild-type (WT) or mutant p53. In some embodiments, p53 binding ligands can bind to a cleft in C-terminus of p53 WT or mutant. In some embodiments, p53 binding ligands can bind p53 (WT or mutant) at the edge of DNA binding domain (site of CDB3 binding). In some embodiments, p53 binding ligands can interact with Asp268 of p53 WT or mutant.

In some embodiments, the p53 mutant is the Y220C mutant. In some embodiments, the p53 binding ligands may interact with Cys220, Asp228, Thr150, Ser227, and Leu145. The p53 binding ligands may interact with Cys220, Asp228, Thr150, Ser227, or Leu145 of the p52 Y220C mutant. In some embodiments, the p53 binding ligands may interact with any combination of Cys220, Asp228, Thr150, Ser227, and Leu145 of the p53 Y220C mutant. The interaction may comprise a covalent binding. For the p53 Y220C mutant, the p53 binding ligand may interact with Cys220. For the p53 Y220C mutant, the p53 binding ligand may interact with Asp228. For p53 binding ligands that react covalently with cysteines, the binding ligand may interact with Cys124, Cys182, Cys220 (in p53 Y220C mutant), Cys229, or Cys277. For p53 binding ligands that react covalently with cysteines, the binding ligands may interact with Cys220 of the p53 Y220C mutant.

Some aspects include a heterobifunctional compound (BFM) comprising: a p53 binding moiety attached to a recruiting ligand that binds a modifier protein, wherein the p53 binding moiety binds to one of a plurality of mutant forms of p53. Some aspects include a heterobifunctional compound comprising: a p53 binding moiety attached to a recruiting ligand that binds a modifier protein. The p53 binding moiety may bind to a mutant form of p53, such as a mutant form of p53 selected from a mutant form described herein. The mutant form may include a substitution such as a Y220C mutation. In some aspects, the modifier protein comprises BRD4. Some aspects include a method of activating a p53 mutant protein, comprising contacting the p53 mutant protein with a compound disclosed herein.

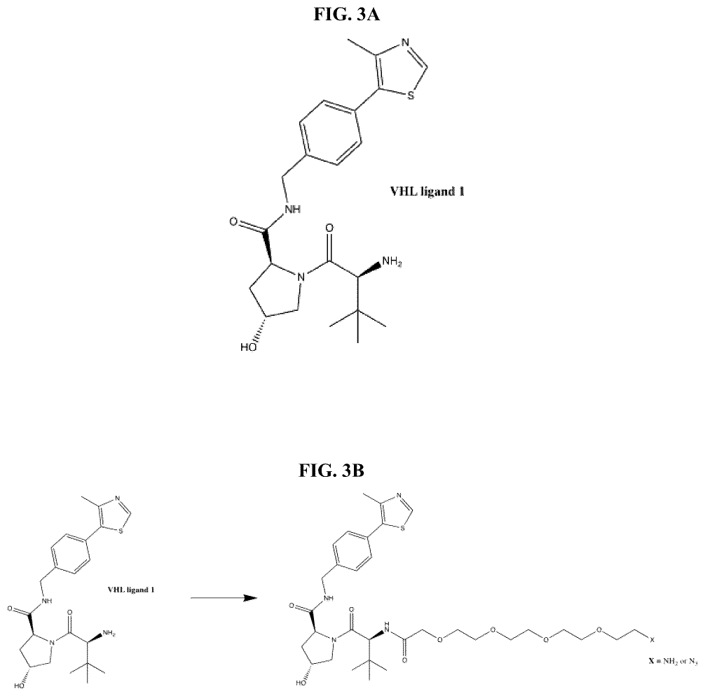

In some embodiments, the target protein includes an E3 ubiquitin ligase. An example of an E3 ubiquitin ligase includes Von Hippel-Lindau tumor suppressor (VHL). VHL is also an example of a tumor suppressor. VHL's E3 ubiquitin ligase may degrade hypoxia-inducible factor (HIF). Some mutations in VHL impair its stability and its ability to degrade HIF. This may lead to tumor growth. Orphan indication is most common in VHL syndrome and clear cell renal cell carcinoma (ccRCC). Previous pharmacochaperones have been ineffective. An example of a targeting ligand that binds to VHL is included in FIG. 3A. FIG. 3B includes an example of a targeting ligand that binds VHL and is attached to a linker. The linker may be further attached to a recruiting ligand, for example, via click or amide coupling. In some embodiments, the compound provided herein targets VHL for modification by a modifier protein described herein. In some embodiments, the modification comprises a post-translation modification of VHL. Further modifications are provided herein.

The VHL may comprise a mutant VHL. The targeting ligand may bind the VHL mutant. Some aspects relate to a heterobifunctional compound (BFM) comprising: a VHL binding moiety attached to a recruiting ligand that binds a modifier protein, wherein the VHL binding moiety binds to one of a plurality of mutant forms of VHL. Some aspects relate to a heterobifunctional compound comprising: a VHL binding moiety attached to a recruiting ligand that binds a modifier protein, wherein the VHL binding moiety binds a mutant form of VHL.

In some embodiments, the target protein includes an enzyme such as a glucosylceramidase. An example of a glucosylceramidase may include β-glucocerebrosidase (GBA). In some embodiments, the compound provided herein targets the glucosylceramidase, e.g., GBA, for modification by a modifier protein described herein. In some embodiments, the modification comprises a post-translation modification of the glucosylceramidase, e.g., GBA. Further modifications are provided herein.

The GBA may comprise a mutant GBA. The targeting ligand may bind the GBA mutant. Some aspects relate to a heterobifunctional compound (BFM) comprising: a GBA binding moiety attached to a recruiting ligand that binds a modifier protein, wherein the GBA binding moiety binds to one of a plurality of mutant forms of GBA. Some aspects relate to a heterobifunctional compound comprising: a GBA binding moiety attached to a recruiting ligand that binds a modifier protein, wherein the GBA binding moiety binds a mutant form of GBA

In some embodiments, the target protein includes a misfolded protein. In some embodiments, the compound provided herein targets the misfolded protein for modification by a modifier protein described herein. In some embodiments, the modification corrects misfolding of the misfolded protein and/or alleviates or compensates for deleterious effects of the misfolded protein. Further modifications are provided herein. In some embodiments, the modification modulates, either directly or indirectly, the half-life of the target protein. In some embodiments, the modification results in a prolonged target protein half-life. In some embodiments, a target protein with a prolonged half-life is capable of being further targeted and/or activated, i.e., in a drug combination approach. In some embodiments, a target protein is weakly activated but has a prolonged half-life, thereby providing the desired effect, e.g., therapeutic effect.

In some embodiments, the target protein comprises P53, VHL, or GBA. In some embodiments, the target protein comprises P53 or VHL. The target protein may include P53. The target protein may include VHL. The target protein may include GBA.

In some embodiments, a target protein comprises a protein associated with a disease state. For example, the target protein may be present, upregulated, or downregulated in the disease state, or a mutation in the target protein may contribute to the disease state. The target protein may play a role in the disease state, such as a deleterious role or a protective role with regard to the disease state. In some embodiments, a target protein comprises a protein with a modification status associated with a disease state. In some embodiments, the compound provided herein targets the target protein for modification by a modifier protein described herein such that the target protein is no longer associated with the disease state. For example, the phosphorylation status of the target protein may associate with the disease state. Examples of disease states may include cancer or a genetic disease. In some embodiments, the disease state includes cancer. In some embodiments, the disease state includes a genetic disease.

The target protein may be in a subject. The target protein may be in a cell. The target protein may be intrinsic to the cell. The target protein may be endogenous to the cell. In some embodiments, the target protein is extrinsic to the cell. In some embodiments, the target protein is exogenous to the cell. The cell may be a cell of the subject.

In some embodiments, the target protein undergoes a change as a result of contact or proximity with the modifier protein. For example, the target protein may undergo a modification. The modification may be an enzymatic modification. The modification may include a post-translational modification. Some examples of modifications may include phosphorylation, palmitoylation, methylation, glycosylation, ligation, sulfonation, ubiquitylation, SUMOylation, fucosylation, sialylation, tyrosylation, acylation, acetylation, or tetradecanoylation. The modification may be added to the target protein or may be removed from the target protein. The modification may include phosphorylation, dephosphorylation, methylation, demethylation, deacylation, deacetylation, glycosylation, deglycosylation, ubiquitylation, or deubiquitylation. The modification may be covalent. The modification may be non-covalent. The target protein may undergo a structural change. Examples of structural changes may include protein folding or misfolding. The structural change may include cleavage. The modification may include changes in cellular localization. The structural change may include changes in oligomerization state.

The target protein may be more active as a result of the change. The target protein may be less active as a result of the change. When the target protein is an enzyme, the enzymatic activity of the target protein may be affected as a result of the change. In some embodiments, the target protein activity is increased or decreased by about 1.5-fold, 2-fold, 3-fold, 4-fold, 5-fold, 10-fold, 20-fold, 50-fold, 100-fold as a result of the change.

### Recruiting Ligands and Modifier Proteins

Disclosed herein, in some embodiments, are recruiting ligands. The recruiting ligand may be configured to bind to a modifier protein. The recruiting ligand may include a moiety that binds the modifier protein. The recruiting ligand may bind the modifier protein. The recruiting ligand may bind the protein or other macromolecule that interacts with a modifier protein. Upon binding, the modifier may induce a change in the target protein. In some embodiments, the recruiting ligand is configured to bind to a modifier protein such that the modifier protein induces a change to the target protein. The recruiting ligand may be a part of a heterobifunctional compound (BFM) such as an OmniTAC compound. The recruiting ligand may connect to a linker or targeting ligand. In some embodiments, the recruiting ligand is incorporated into an in vivo engineered protein.

The recruiting ligand may include a small molecule. In some embodiments, the recruiting ligand comprises a small molecule moiety. In some embodiments, the recruiting ligand has a molecular weight of 200, 300, 400, 500, 600, 700, 800, 900, 1000, 1250, 1500, 1750, 2000, 2250, or 2500 daltons, or a range defined by any two of the aforementioned numbers of daltons.

Disclosed herein, in some embodiments, are recruiting ligands that bind a modifier protein. The recruiting ligand may bind directly to the modifier protein. In some embodiments, the binding between the recruiting ligand and the modifier protein is covalent. The covalent binding may be with a cysteine, lysine, methionine, or any other reactive residue of the modifier protein. In some embodiments, the binding between the recruiting ligand and the modifier protein is non-covalent.

Disclosed herein R² comprises a recruiting ligand (RL) that binds to modifier proteins. The recruiting ligands can be synthesized from parent ligands. Parent ligands are small molecule binders for modifier proteins; these binders can be activators, inhibitors or have no effect on the modifier protein. Recruiting ligands comprise parent ligands with a chemical handle to attach to L or R¹ directly through appropriate chemical transformation. In some cases, the parent ligand may already contain a suitable chemical handle that can be utilized without affecting the binding ability of the ligand. In some embodiments, the recruiting ligand is the parent ligand. In some embodiments, the recruiting ligand is a truncated analogue of the parent ligand. In some embodiments, the recruiting ligand is an analogue of the parent ligand where a functional group or ring has been replaced by an appropriate bioisostere. In some cases, R² is designed as an analogue of the parent ligand by attaching a chemical handle that (1) is solvent exposed and (2) does not impair the binding to the modifier protein. In some embodiments, the exit vector is defined as the orientation and area projected by attachment of a linker to a parent ligand. One parent ligand can lead to several R² designs with varied exit vector and/or chemical handles. The attachment point should minimize impact on the binding mode and affinity of the parent ligand. To do so, the following guidelines can be followed:

### (a) Exit Vector Choice

if crystal structures data is available, the binding mode is analyzed to identify solvent exposed regions of the ligand.
if structure activity relationship (SAR) data is available, it should be used to determine regions of the ligands that can be modified without resulting in large drop of binding affinity (measured by direct binding, e.g. K_{D}, or through effectives concentrations e.g. EC50, IC50 etc. . . . ).
if the parent ligands have previously been described as part of bifunctional molecules (BFMs), the described attachment point will be identified.
if no structural and SAR data is available, then a minimum of 3 exit vectors will be explored as part of tool compound to empirically determine a suitable R².

### (b) Chemical Handle Choice

a chemical group that can be reacted with compatible functional groups to form a new chemical bond in high yields by anyone skilled in the field of organic chemistry. Such reactions include amide coupling, Click reaction, sulfonamide formation, alkylation, reductive amination . . .
the chemical handle can be for example: carboxylic acid, alkyne, amines, azide, isocyanide, sulfonyl chloride, alkyl bromide, alkyl chloride, alkyl iodide, alkyl mesylate, aldehydes, ketone.
The chemical handle should be chosen such that it is compatible with other functional groups within the parent ligand.

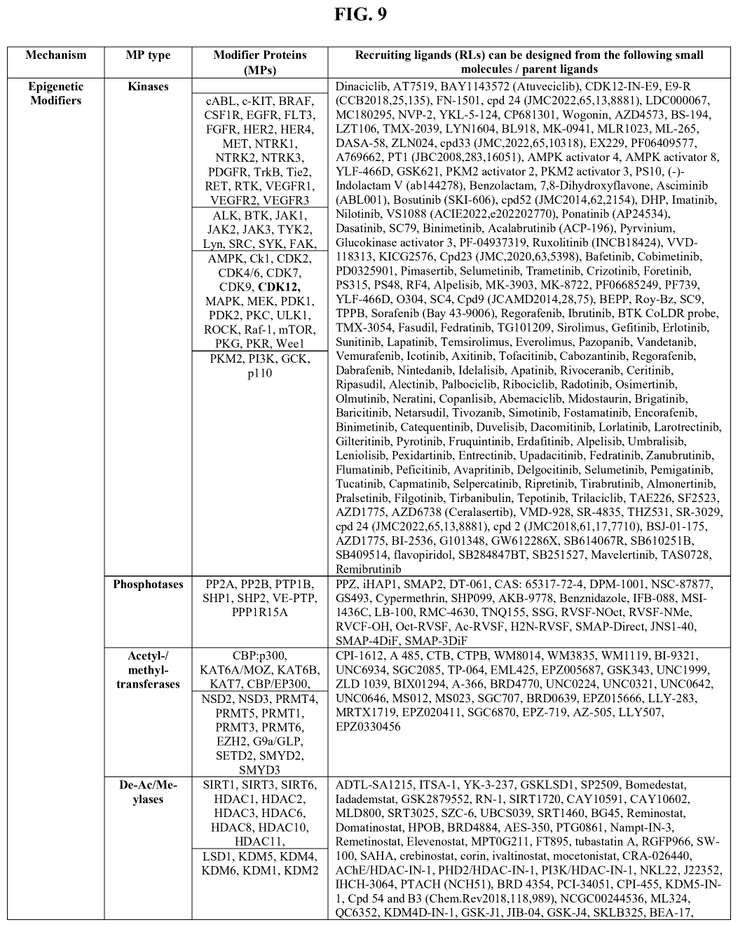

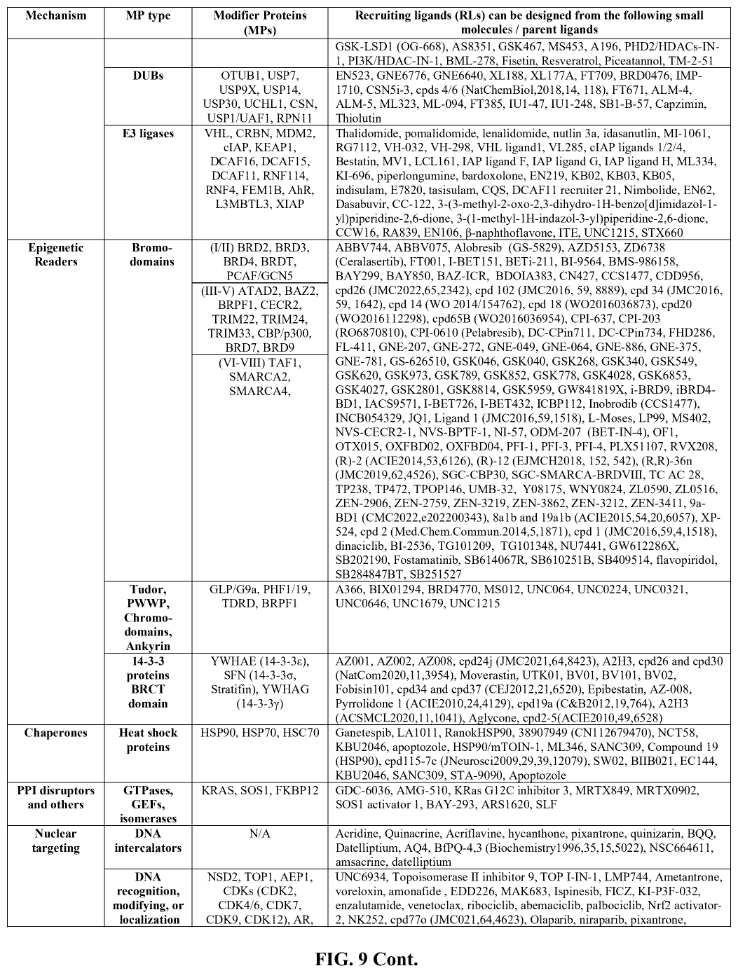

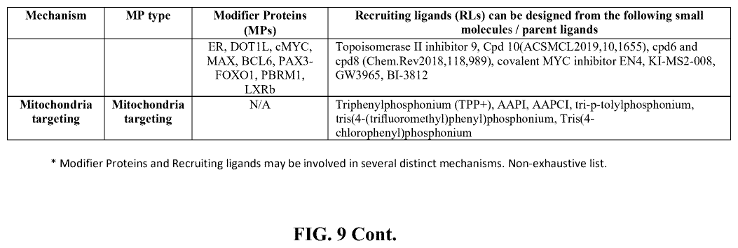

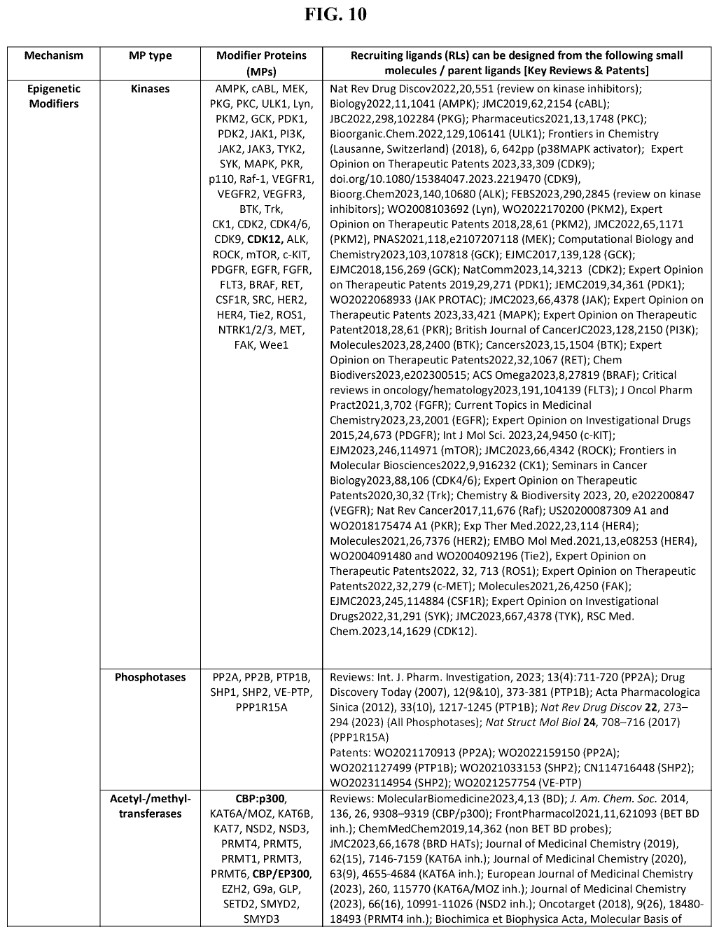

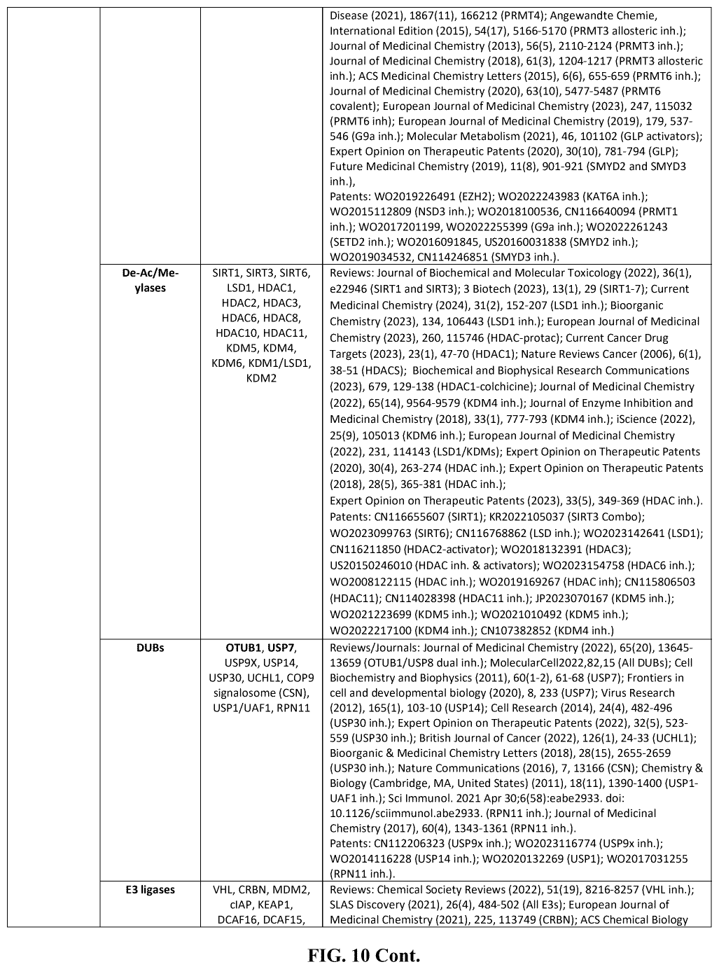

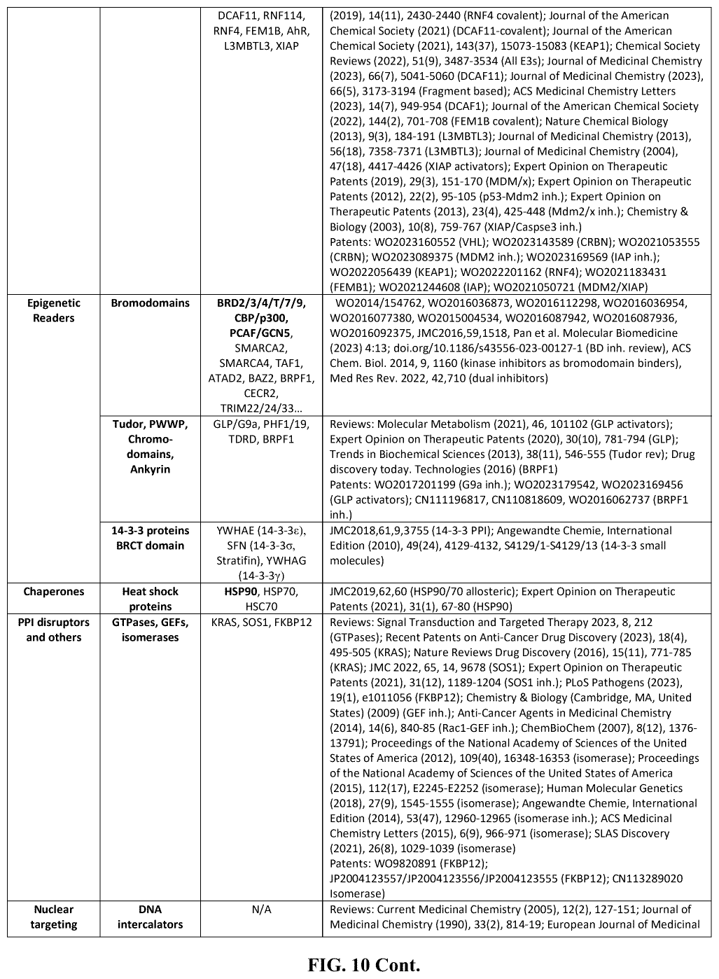

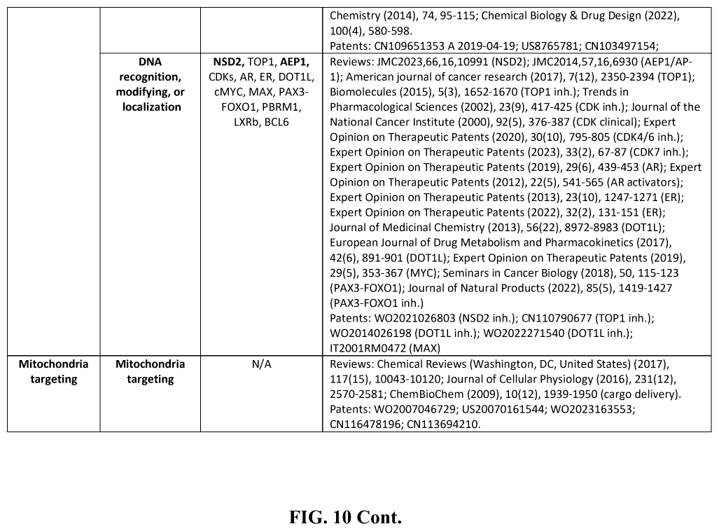

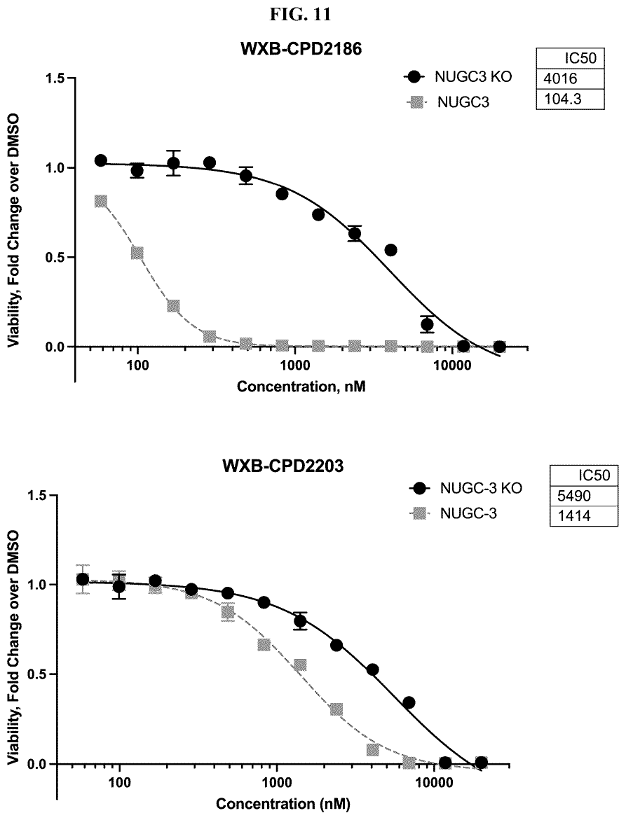

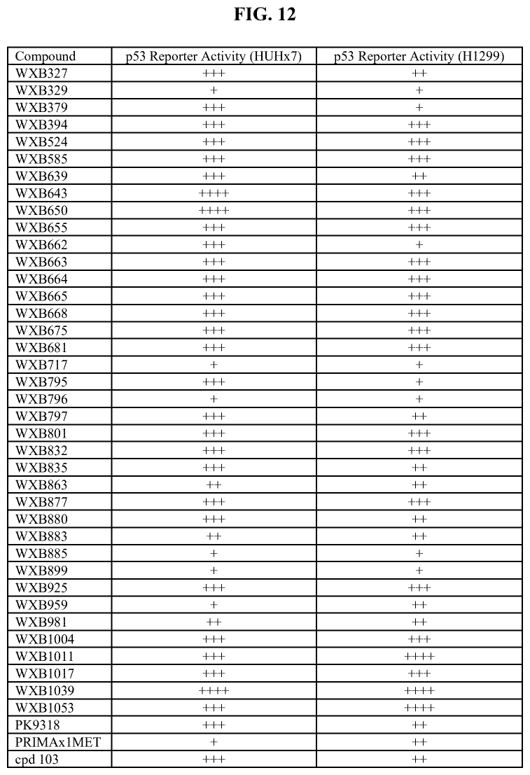

In some embodiments, the parent ligand is selected from the compounds listed in FIG. 9 or FIG. 10. In some embodiments, the parent ligand is selected from the compounds listed in FIG. 9. In some embodiments, the parent ligand is selected from the compounds listed in FIG. 10.

In some embodiments, the parent ligand is:

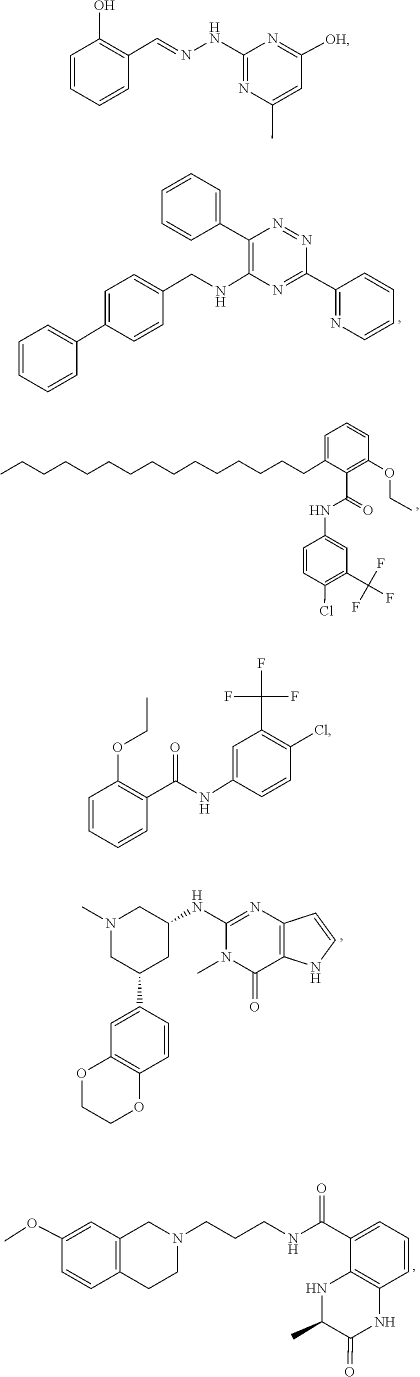

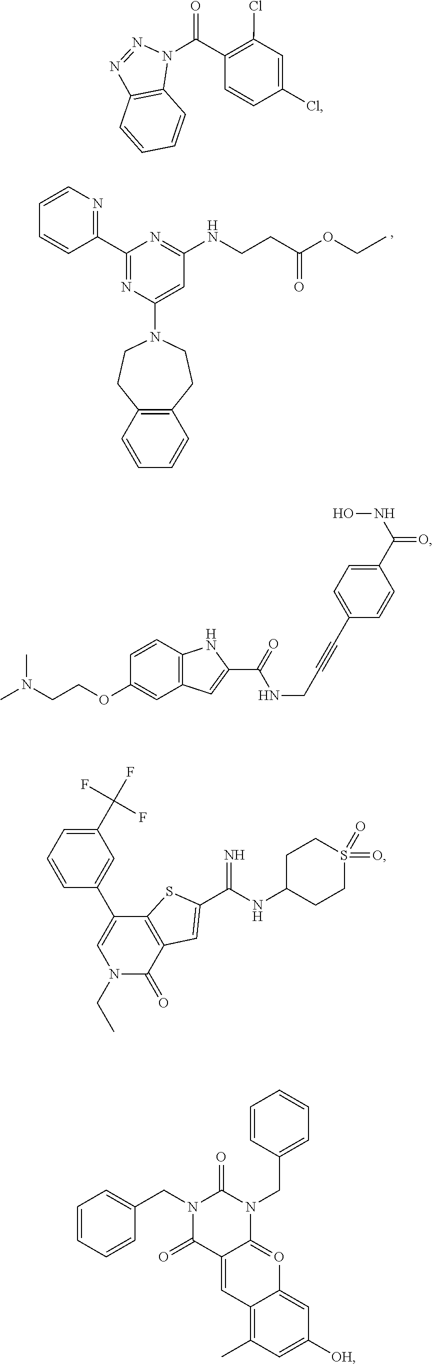

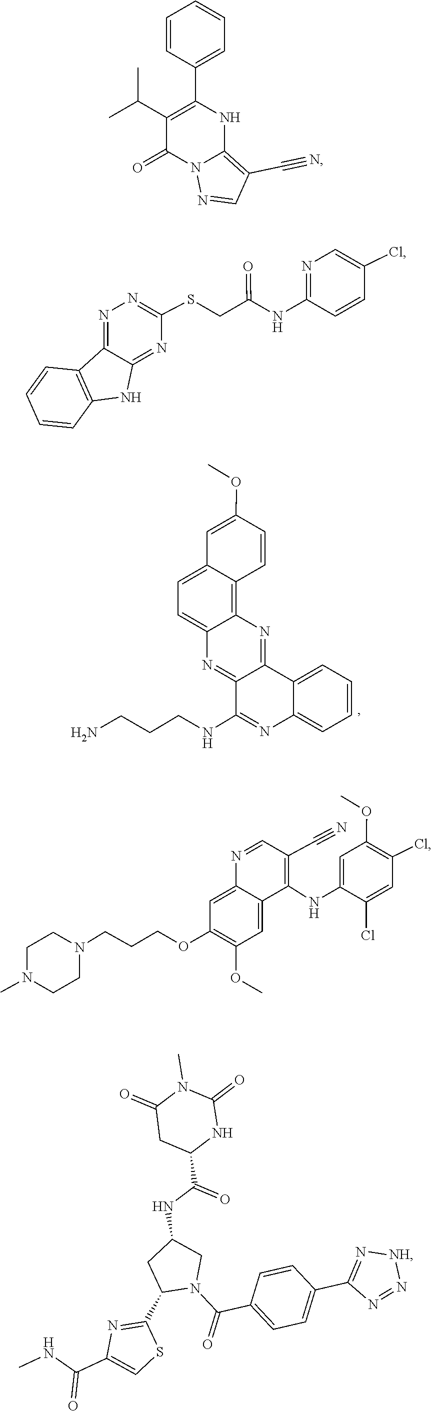 [SMILES: CC(C)c1c(-c2ccccc2)[nH]c2c(C#N)cnn2c1=O ; O=C(CSc1nnc2c(n1)[nH]c1ccccc12)Nc1ccc(Cl)cn1 ; COc1ccc2c(ccc3nc4c(NCCCN)nc5ccccc5c4nc32)c1 ; COc1cc(Nc2c(C#N)cnc3cc(OCCCN4CCN(C)CC4)c(OC)cc23)c(Cl)cc1Cl ; CNC(=O)c1csc([C@@H]2C[C@H](NC(=O)[C@@H]3CC(=O)N(C)C(=O)N3)CN2C(=O)c2ccc(-c3nn[nH]n3)cc2)n1]

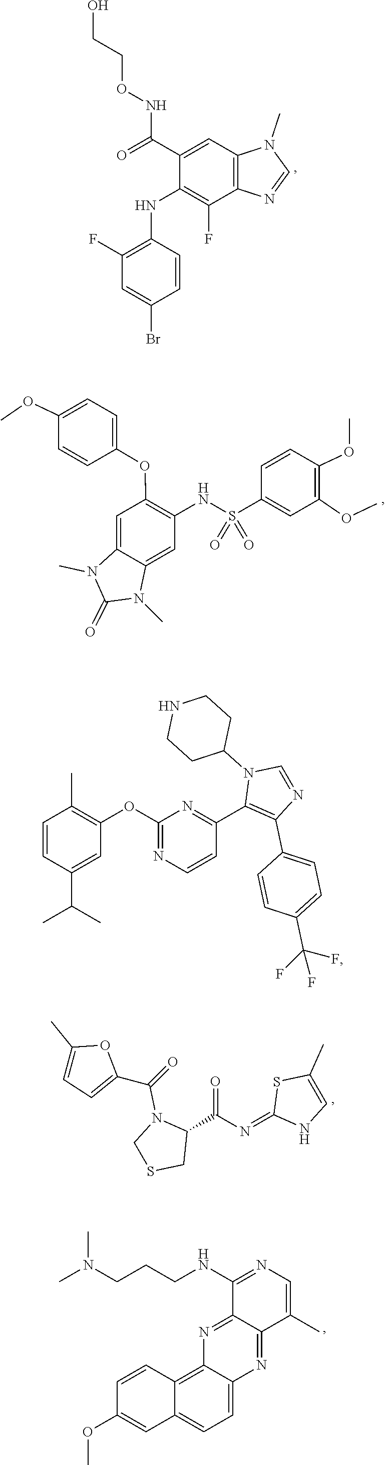

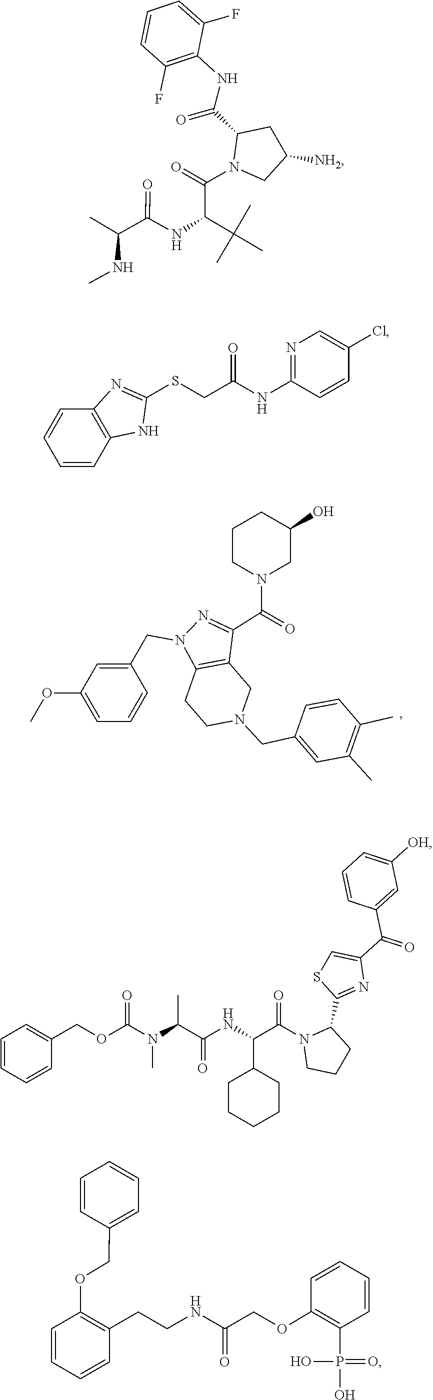

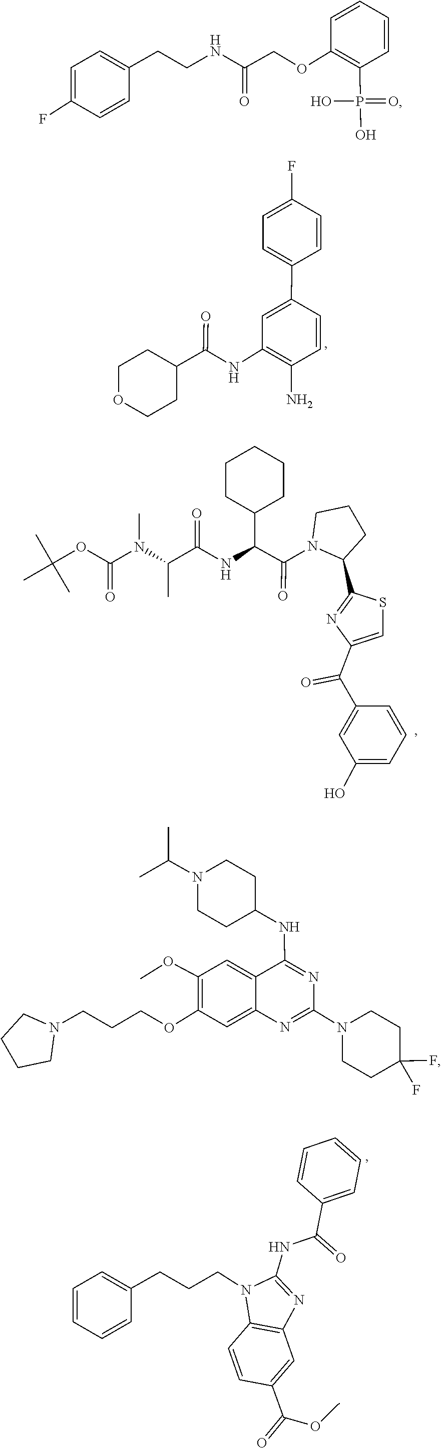 [SMILES: O=C(COc1ccccc1P(=O)(O)O)NCCc1ccc(F)cc1 ; Nc1ccc(-c2ccc(F)cc2)cc1NC(=O)C1CCOCC1 ; C[C@@H](C(=O)N[C@H](C(=O)N1CCC[C@H]1c1nc(C(=O)c2cccc(O)c2)cs1)C1CCCCC1)N(C)C(=O)OC(C)(C)C ; COc1cc2c(NC3CCN(C(C)C)CC3)nc(N3CCC(F)(F)CC3)nc2cc1OCCCN1CCCC1 ; COC(=O)c1ccc2c(c1)nc(NC(=O)c1ccccc1)n2CCCc1ccccc1]

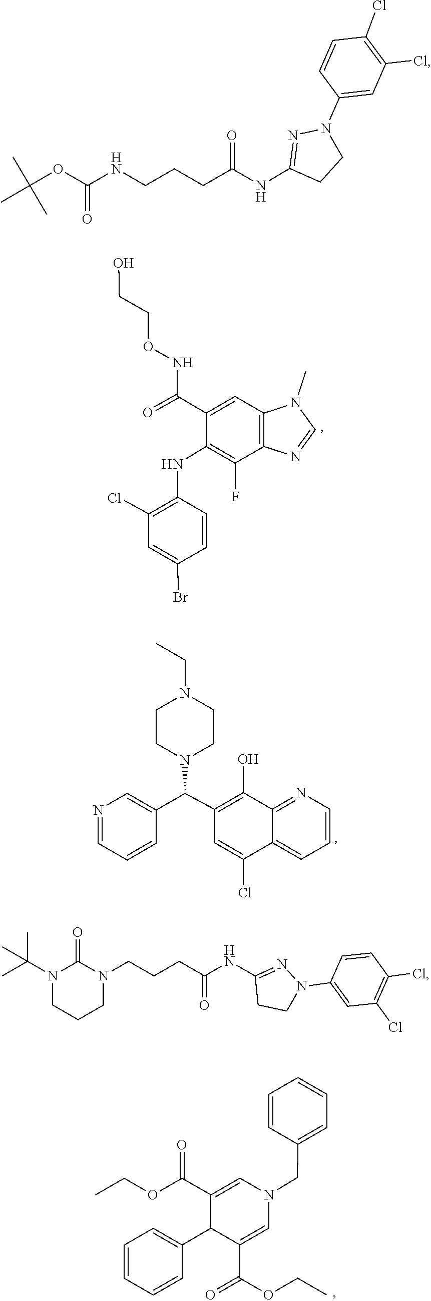 [SMILES: CC(C)(C)OC(=O)NCCCC(=O)NC1=NN(c2ccc(Cl)c(Cl)c2)CC1 ; Cn1cnc2c(F)c(Nc3ccc(Br)cc3Cl)c(C(=O)NOCCO)cc21 ; CCN1CCN([C@@H](c2cccnc2)c2cc(Cl)c3cccnc3c2O)CC1 ; CC(C)(C)N1CCCN(CCCC(=O)NC2=NN(c3ccc(Cl)c(Cl)c3)CC2)C1=O ; CCOC(=O)C1=CN(Cc2ccccc2)C=C(C(=O)OCC)C1c1ccccc1]

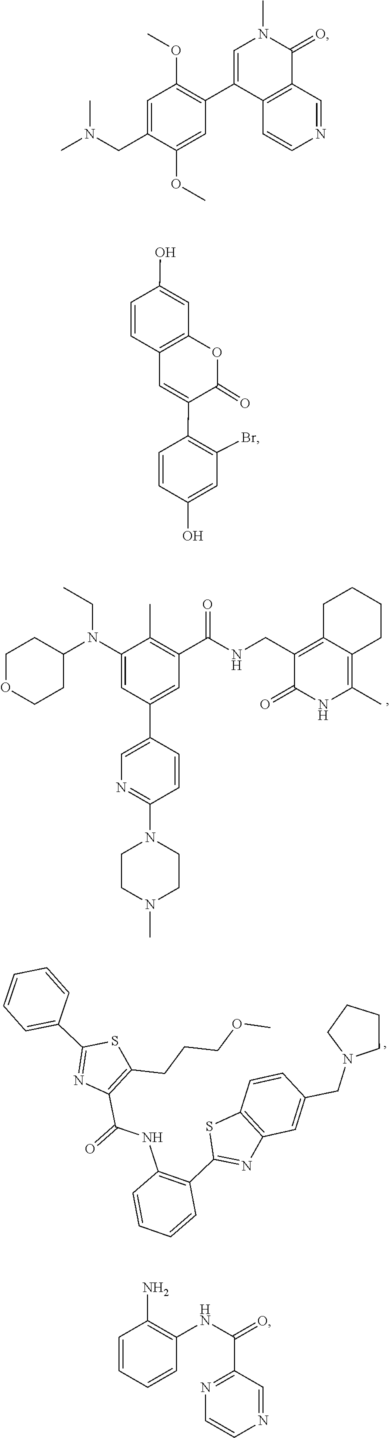 [SMILES: COc1cc(-c2cn(C)c(=O)c3cnccc23)c(OC)cc1CN(C)C ; O=c1oc2cc(O)ccc2cc1-c1ccc(O)cc1Br ; CCN(c1cc(-c2ccc(N3CCN(C)CC3)nc2)cc(C(=O)NCc2c3c(c(C)[nH]c2=O)CCCC3)c1C)C1CCOCC1 ; COCCCc1sc(-c2ccccc2)nc1C(=O)Nc1ccccc1-c1nc2cc(CN3CCCC3)ccc2s1 ; Nc1ccccc1NC(=O)c1cnccn1]

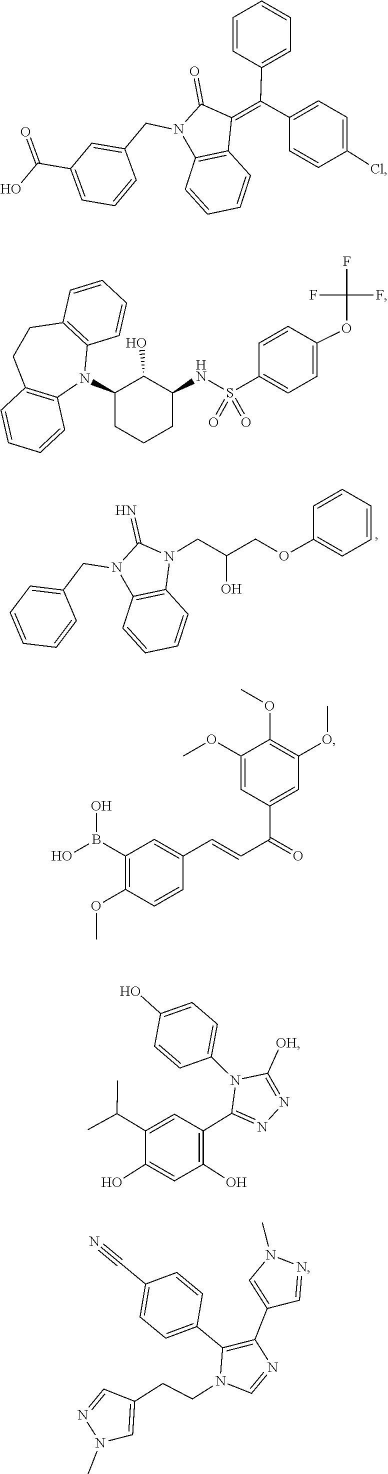

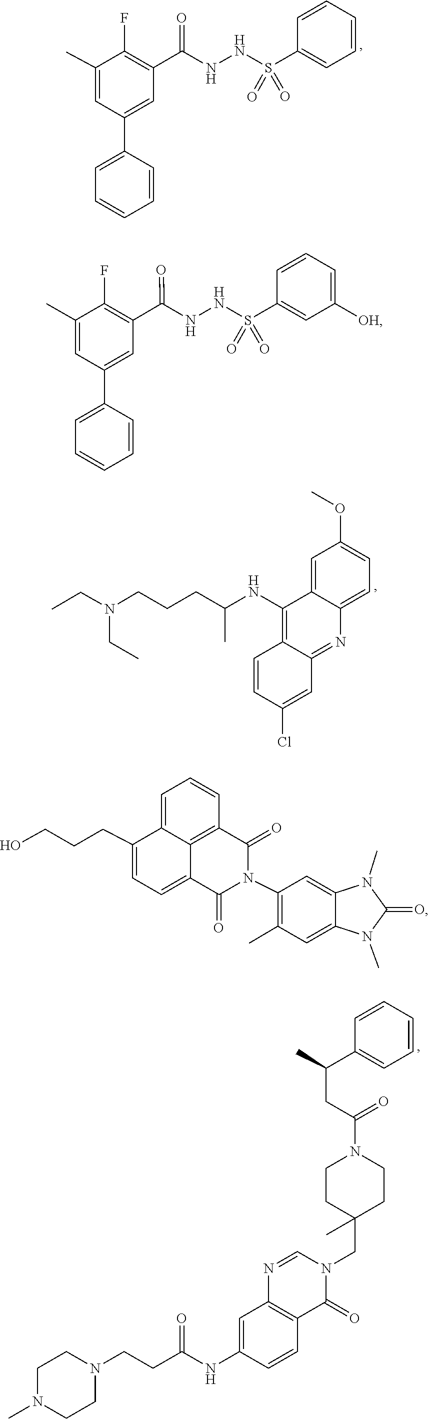

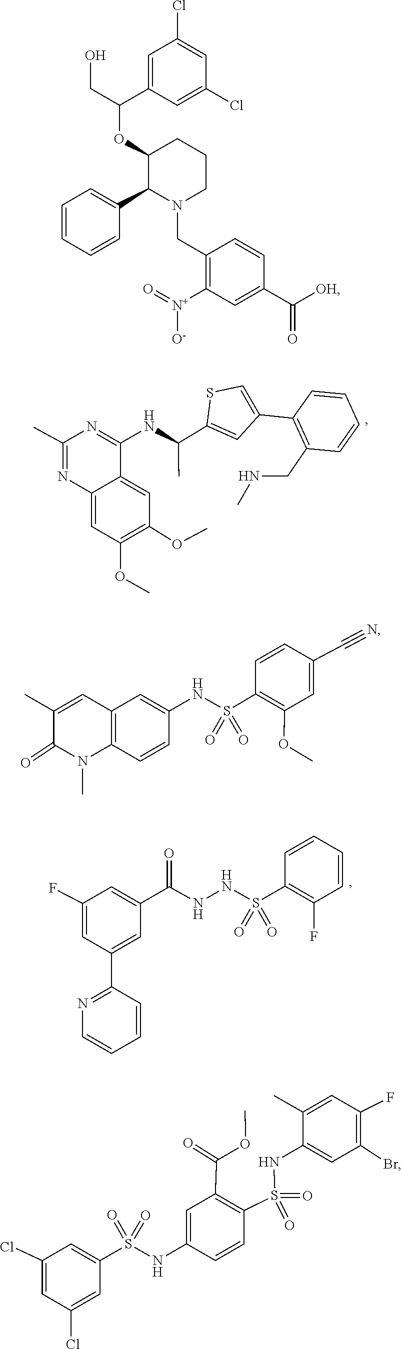 [SMILES: O=C(O)c1ccc(CN2CCC[C@H](OC(CO)c3cc(Cl)cc(Cl)c3)[C@@H]2c2ccccc2)c([N+](=O)[O-])c1 ; CNCc1ccccc1-c1csc([C@@H](C)Nc2nc(C)nc3cc(OC)c(OC)cc23)c1 ; COc1cc(C#N)ccc1S(=O)(=O)Nc1ccc2c(c1)cc(C)c(=O)n2C ; O=C(NNS(=O)(=O)c1ccccc1F)c1cc(F)cc(-c2ccccn2)c1 ; COC(=O)c1cc(NS(=O)(=O)c2cc(Cl)cc(Cl)c2)ccc1S(=O)(=O)Nc1cc(Br)c(F)cc1C]

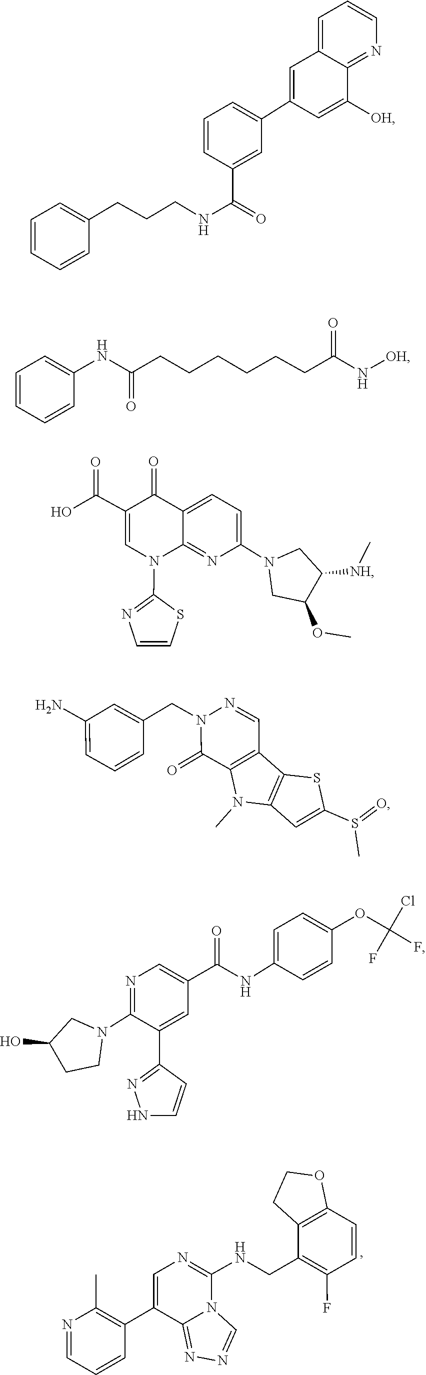 [SMILES: O=C(NCCCc1ccccc1)c1cccc(-c2cc(O)c3ncccc3c2)c1 ; O=C(CCCCCCC(=O)Nc1ccccc1)NO ; CN[C@H]1CN(c2ccc3c(=O)c(C(=O)O)cn(-c4nccs4)c3n2)C[C@@H]1OC ; Cn1c2cc(S(C)=O)sc2c2cnn(Cc3cccc(N)c3)c(=O)c21 ; O=C(Nc1ccc(OC(F)(F)Cl)cc1)c1cnc(N2CC[C@@H](O)C2)c(-c2cc[nH]n2)c1 ; Cc1ncccc1-c1cnc(NCc2c(F)ccc3c2CCO3)n2cnnc12]

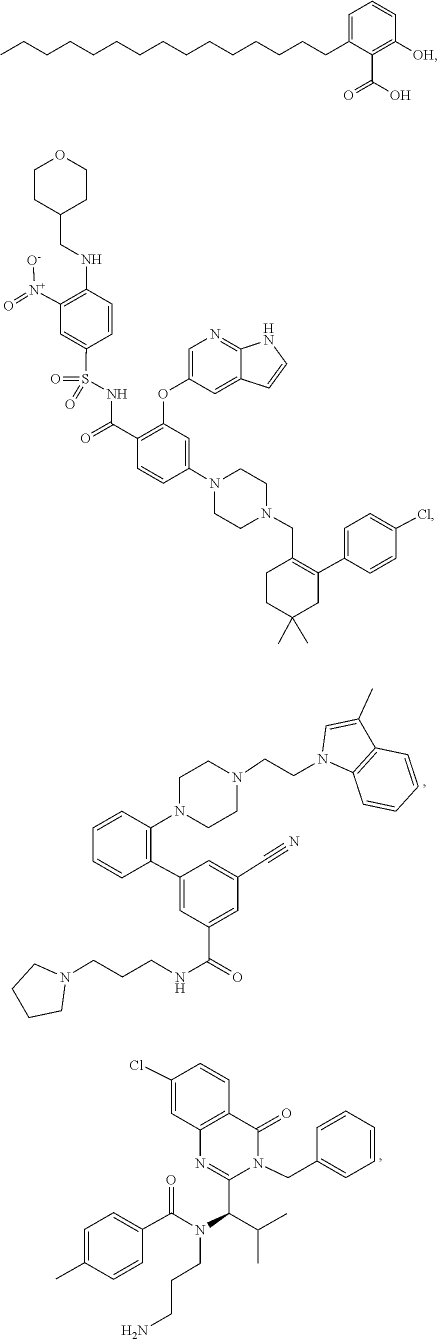 [SMILES: CCCCCCCCCCCCCCCc1cccc(O)c1C(=O)O ; CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1 ; Cc1cn(CCN2CCN(c3ccccc3-c3cc(C#N)cc(C(=O)NCCCN4CCCC4)c3)CC2)c2ccccc12 ; Cc1ccc(C(=O)N(CCCN)[C@@H](c2nc3cc(Cl)ccc3c(=O)n2Cc2ccccc2)C(C)C)cc1]

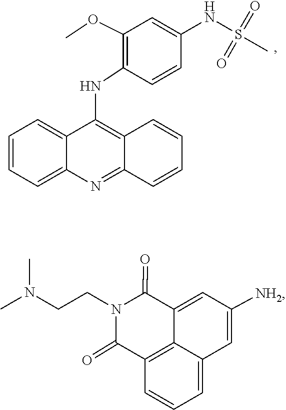

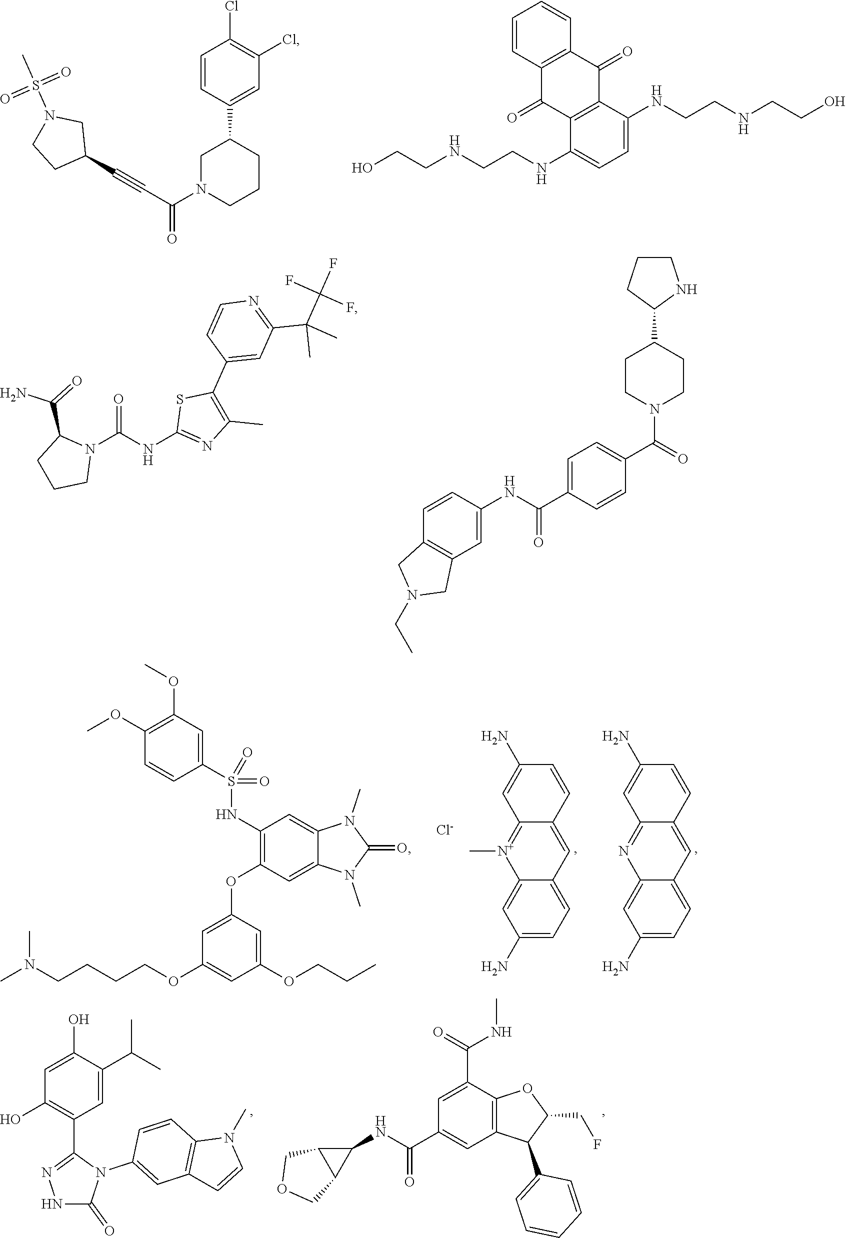

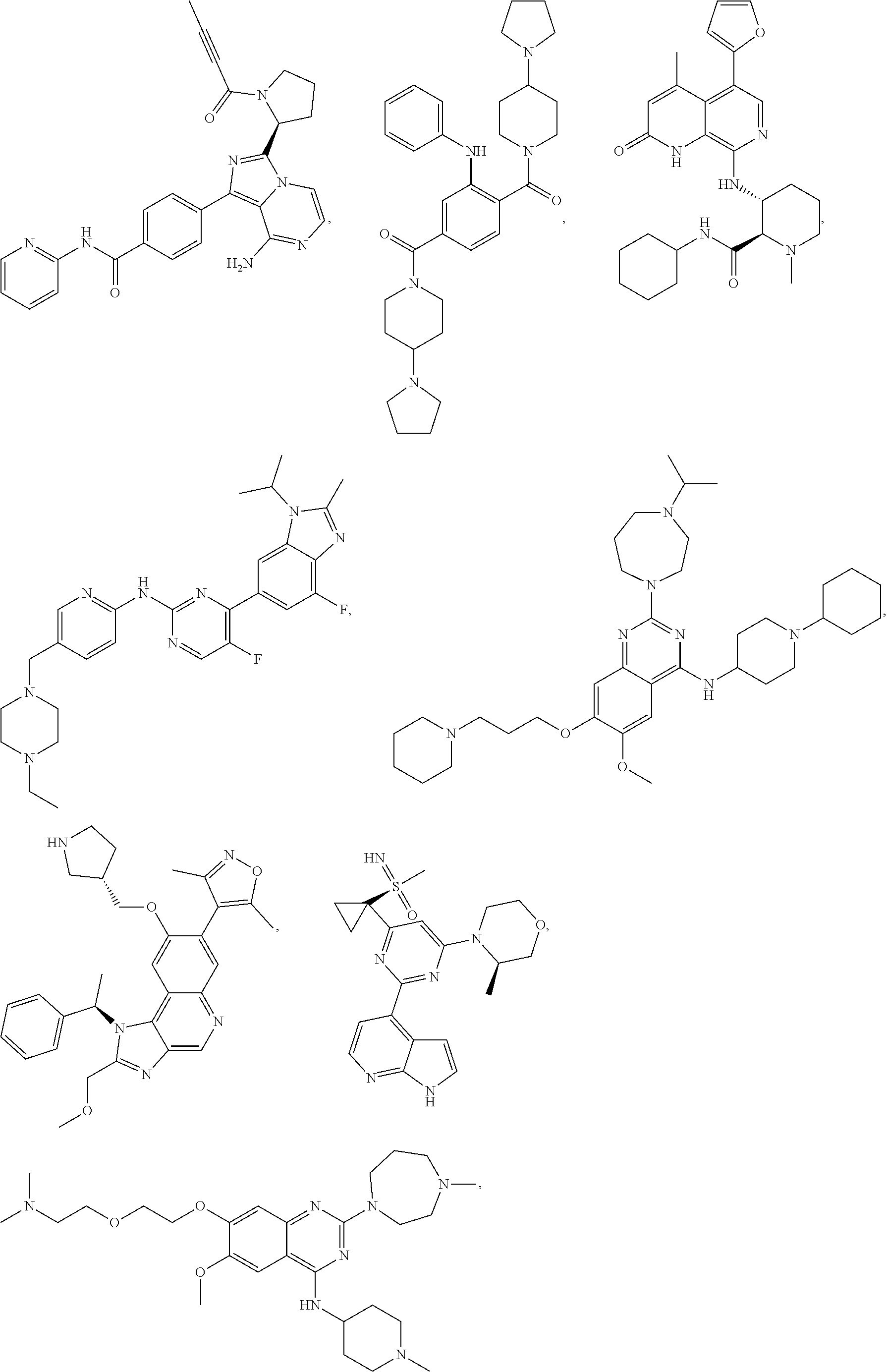

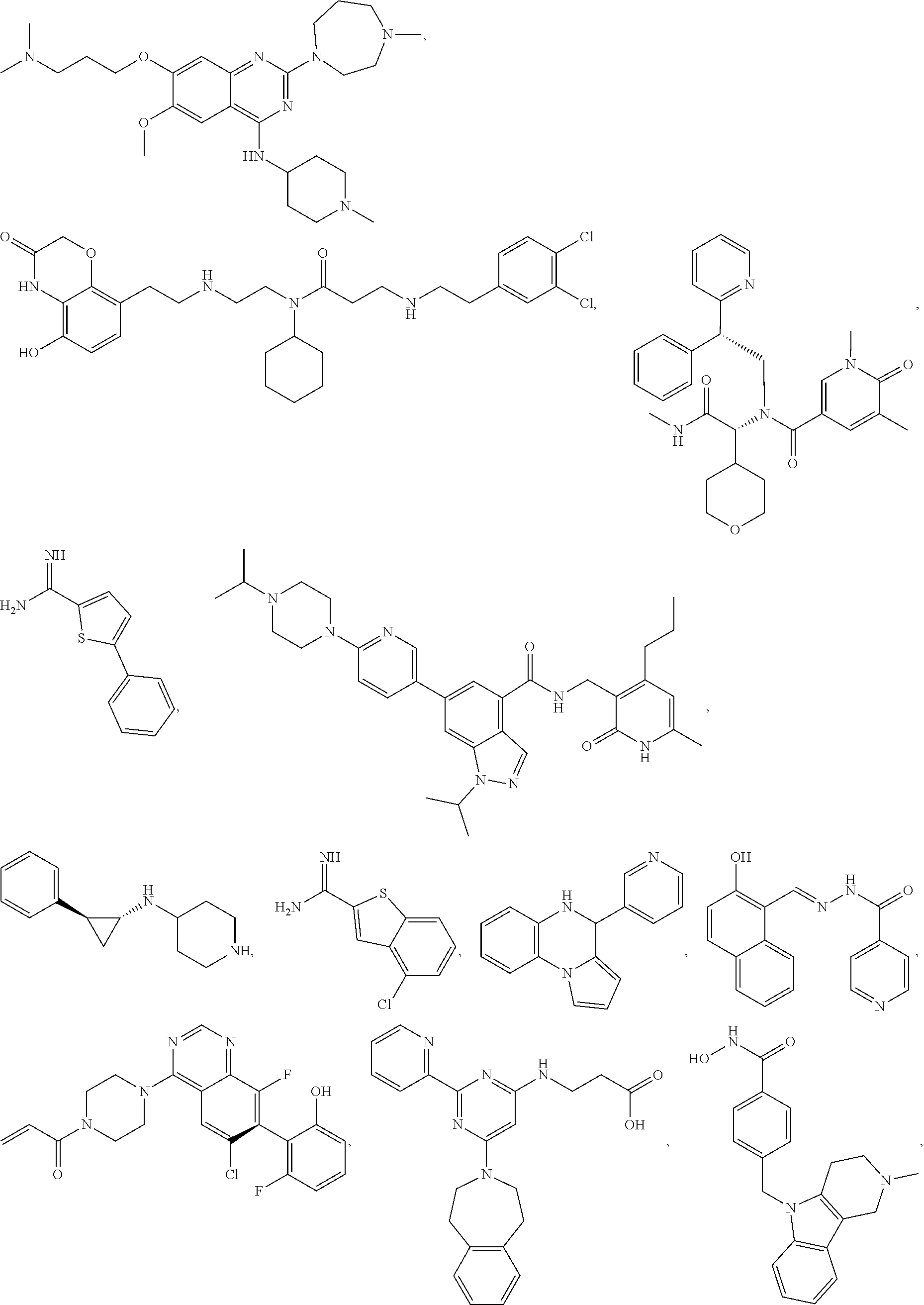

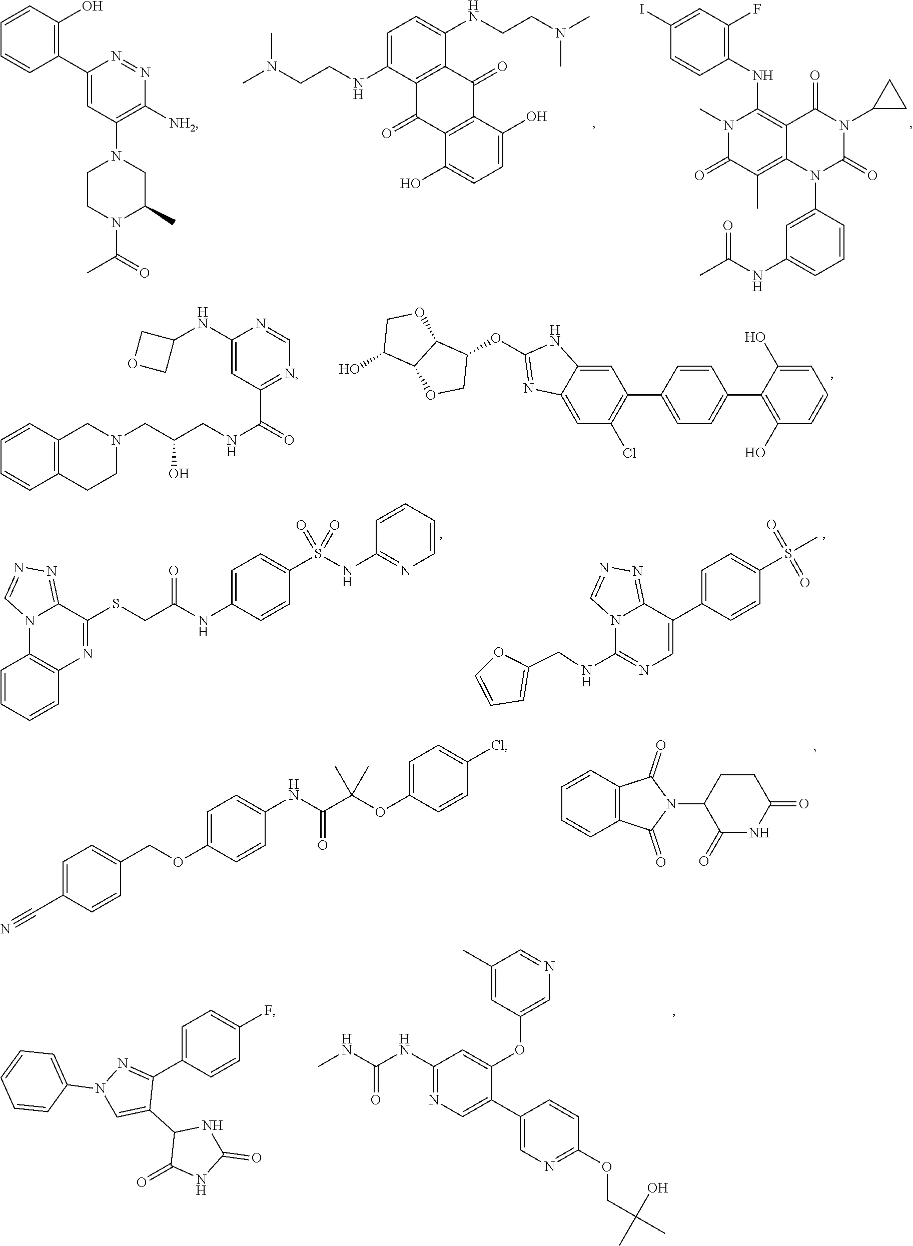

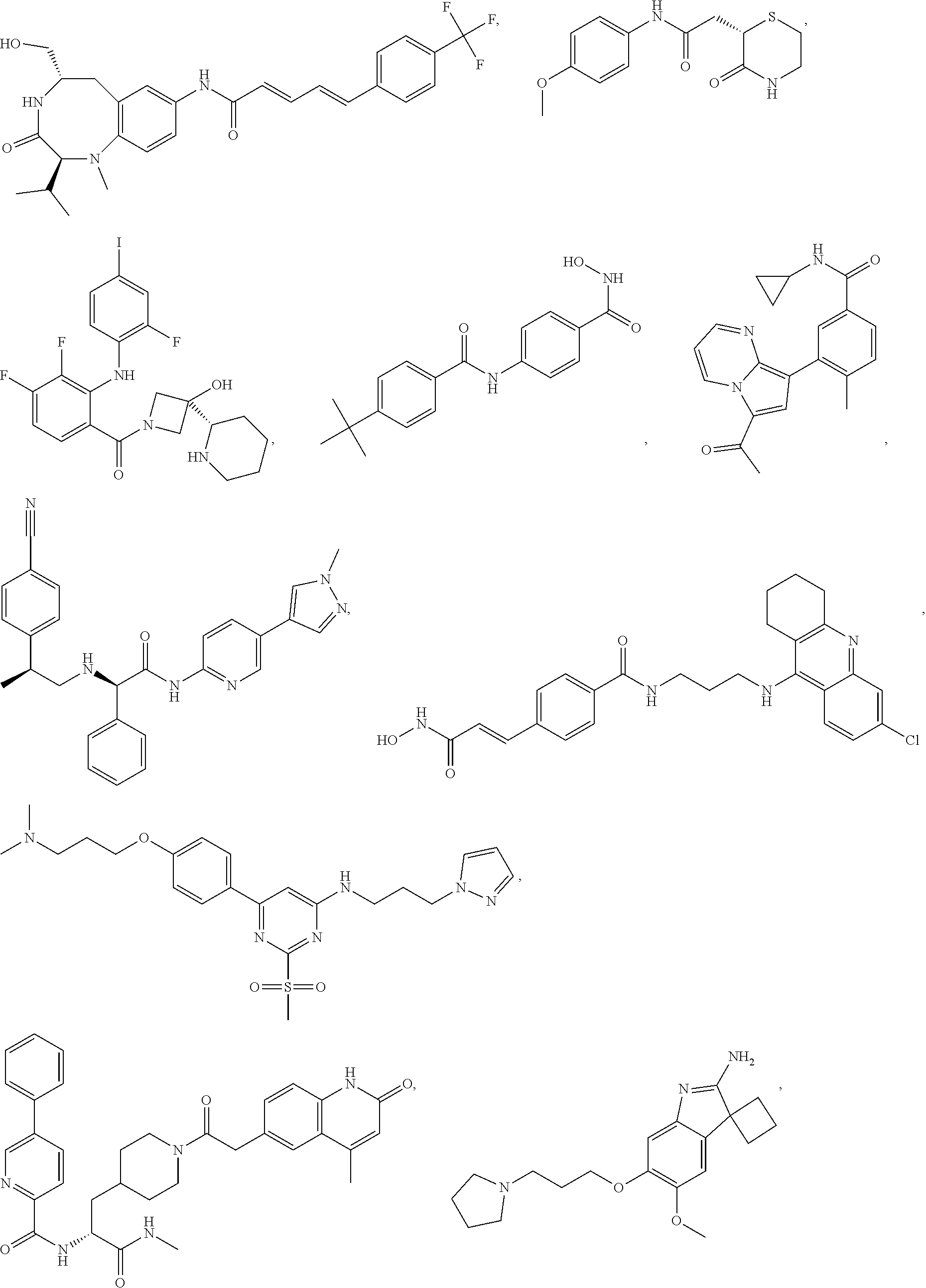

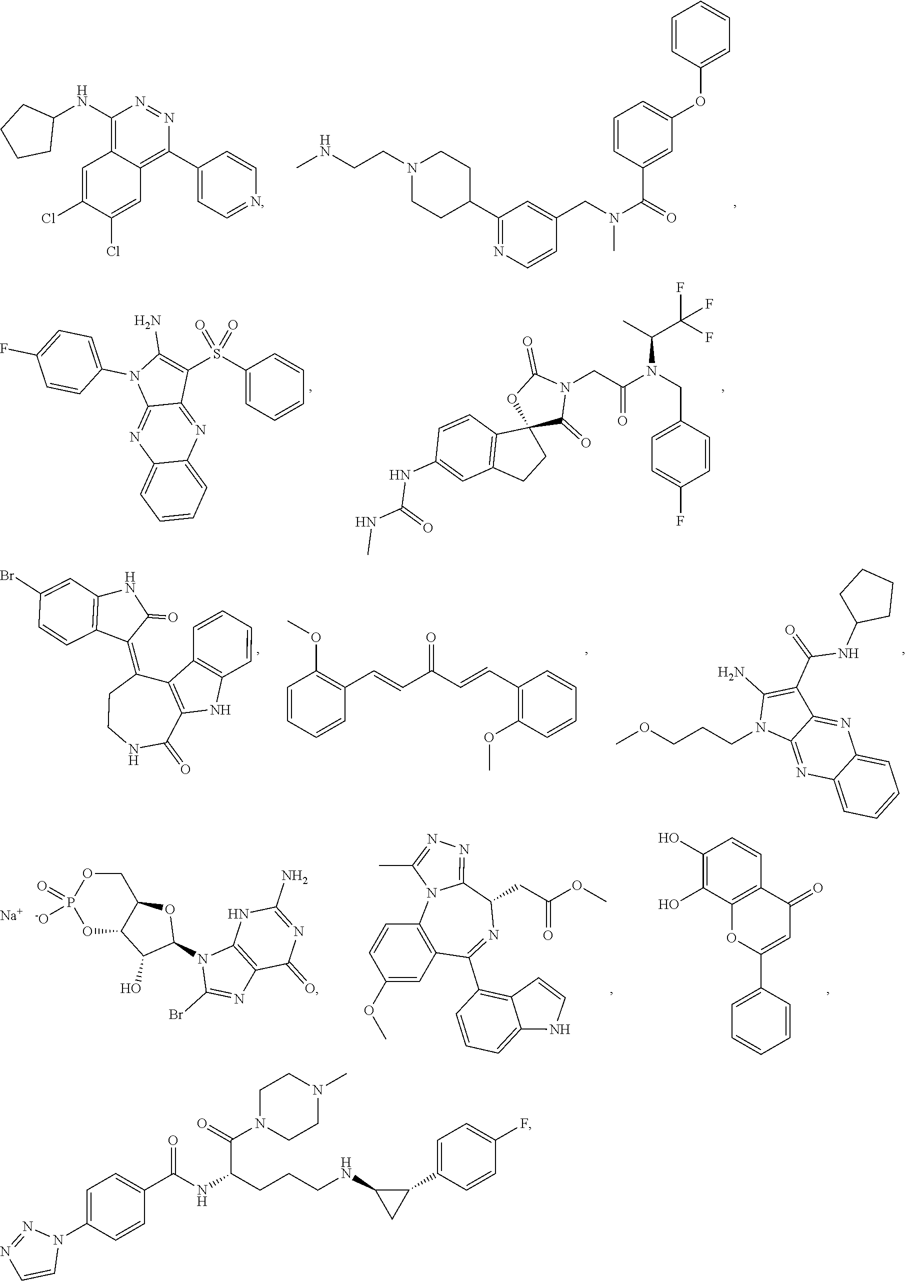

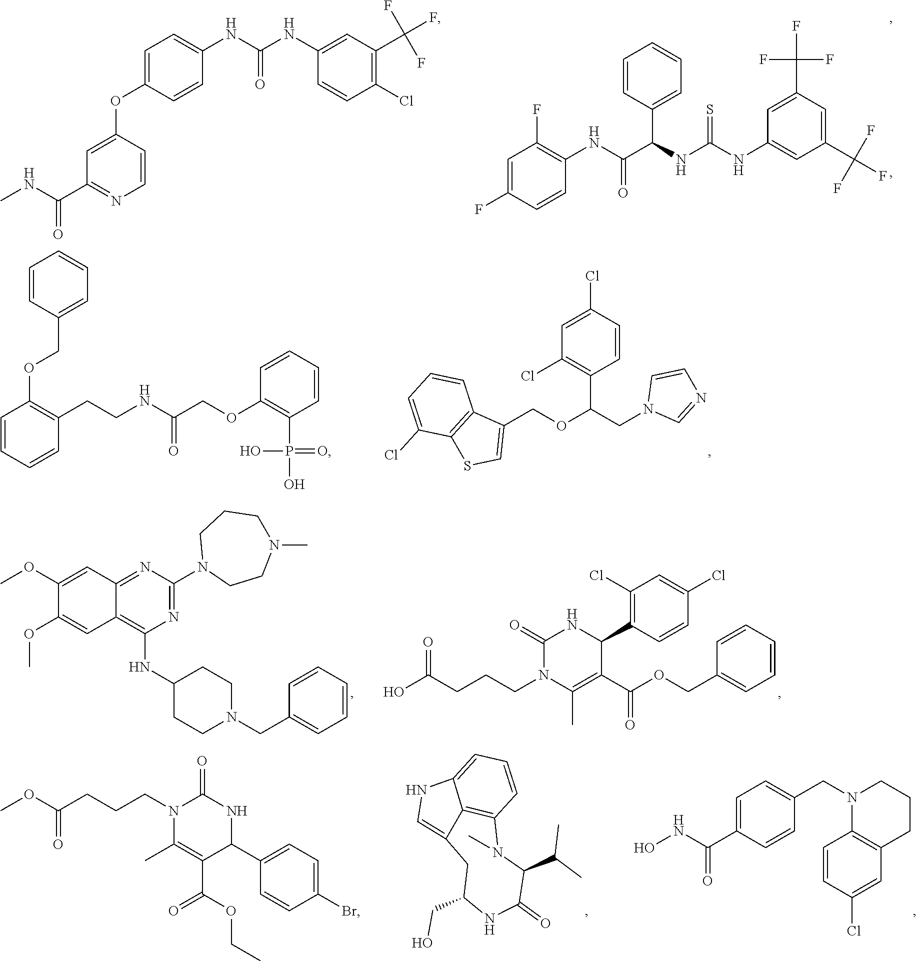

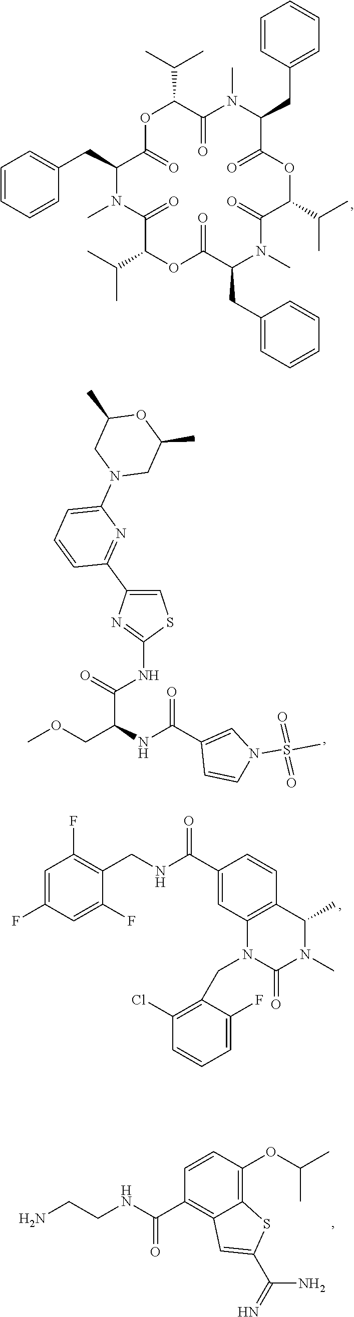

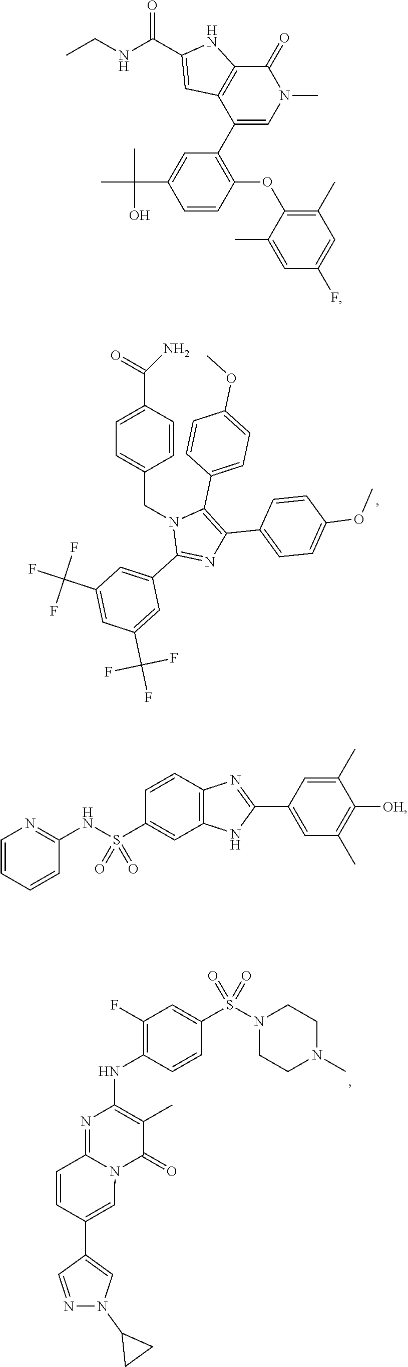 [SMILES: CCNC(=O)c1cc2c(-c3cc(C(C)(C)O)ccc3Oc3c(C)cc(F)cc3C)cn(C)c(=O)c2[nH]1 ; COc1ccc(-c2nc(-c3cc(C(F)(F)F)cc(C(F)(F)F)c3)n(Cc3ccc(C(N)=O)cc3)c2-c2ccc(OC)cc2)cc1 ; Cc1cc(-c2nc3ccc(S(=O)(=O)Nc4ccccn4)cc3[nH]2)cc(C)c1O ; Cc1c(Nc2ccc(S(=O)(=O)N3CCN(C)CC3)cc2F)nc2ccc(-c3cnn(C4CC4)c3)cn2c1=O]

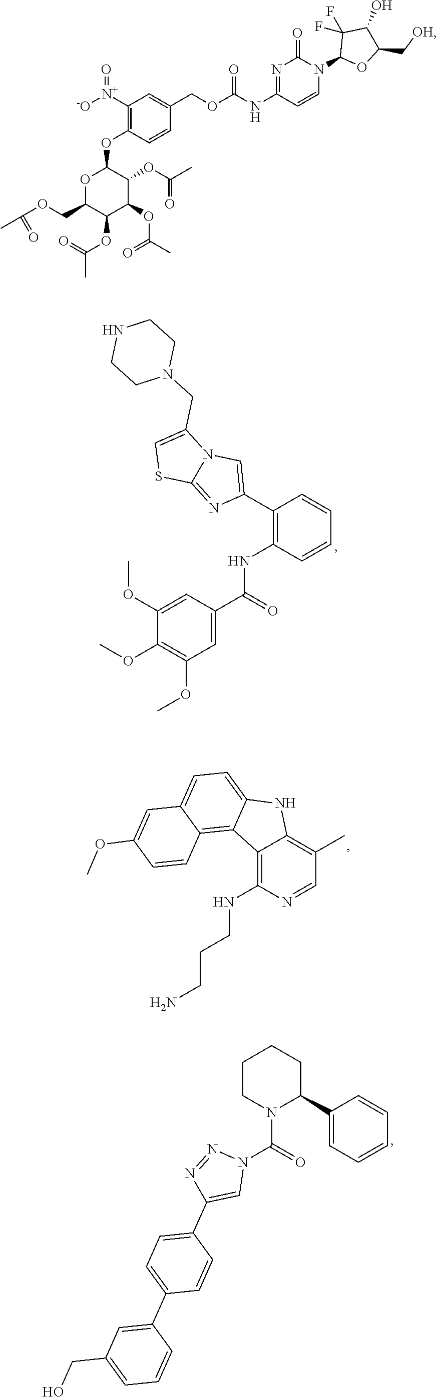

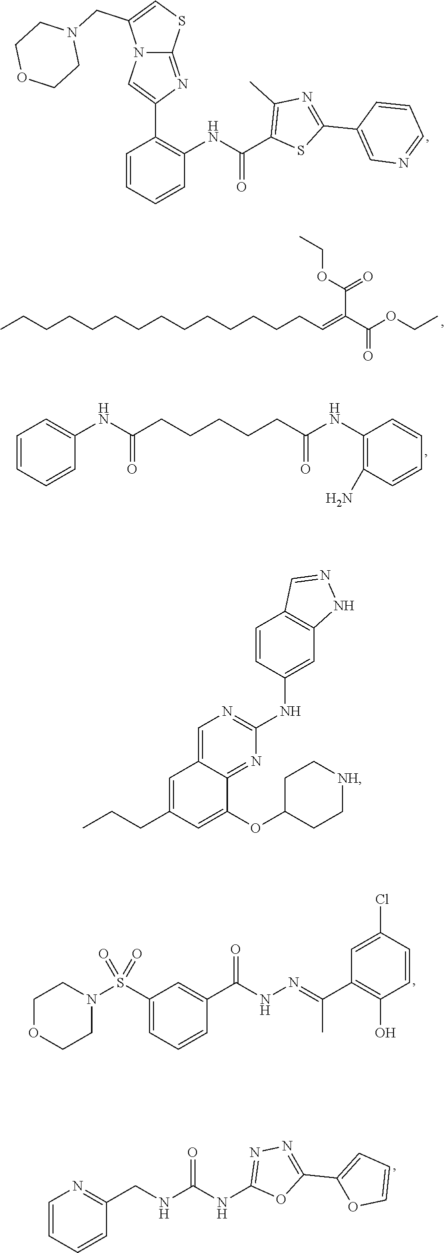

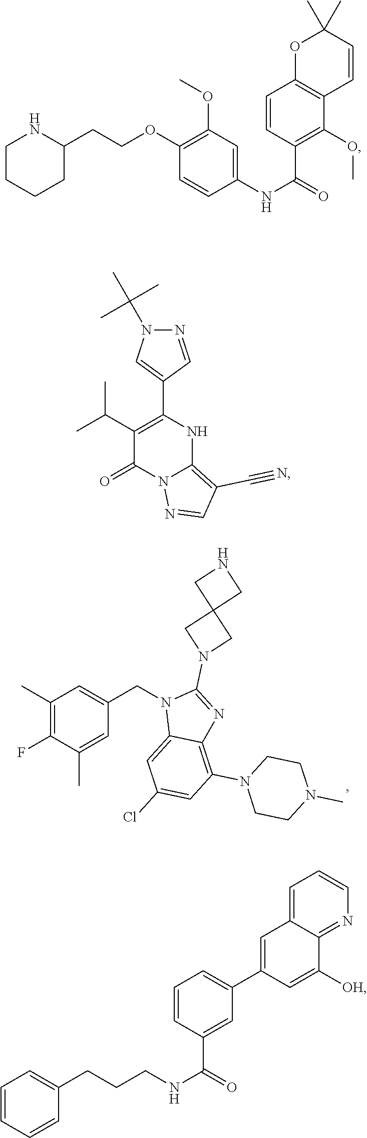 [SMILES: COc1cc(NC(=O)c2ccc3c(c2OC)C=CC(C)(C)O3)ccc1OCCC1CCCCN1 ; CC(C)c1c(-c2cnn(C(C)(C)C)c2)[nH]c2c(C#N)cnn2c1=O ; Cc1cc(Cn2c(N3CC4(CNC4)C3)nc3c(N4CCN(C)CC4)cc(Cl)cc32)cc(C)c1F ; O=C(NCCCc1ccccc1)c1cccc(-c2cc(O)c3ncccc3c2)c1]

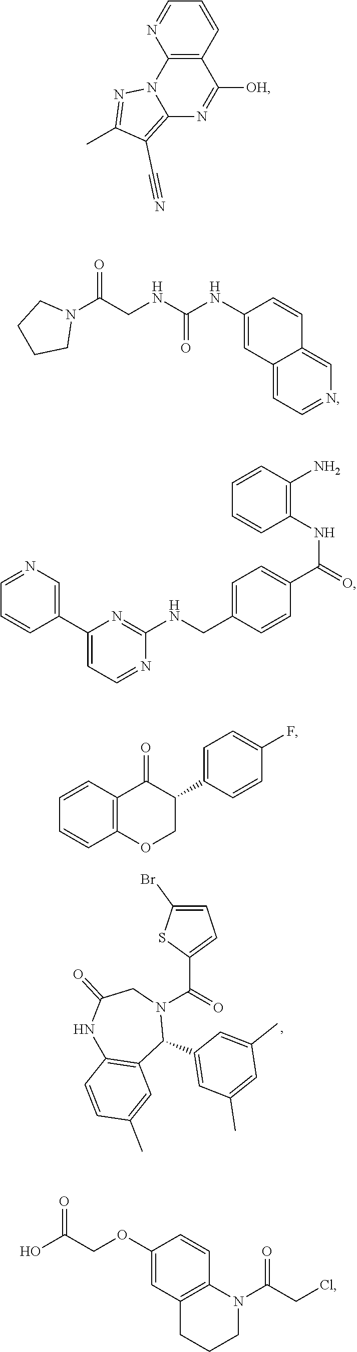

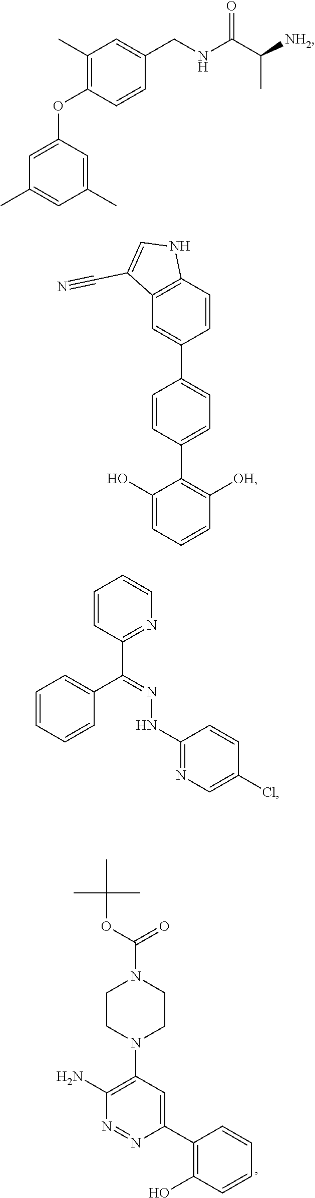

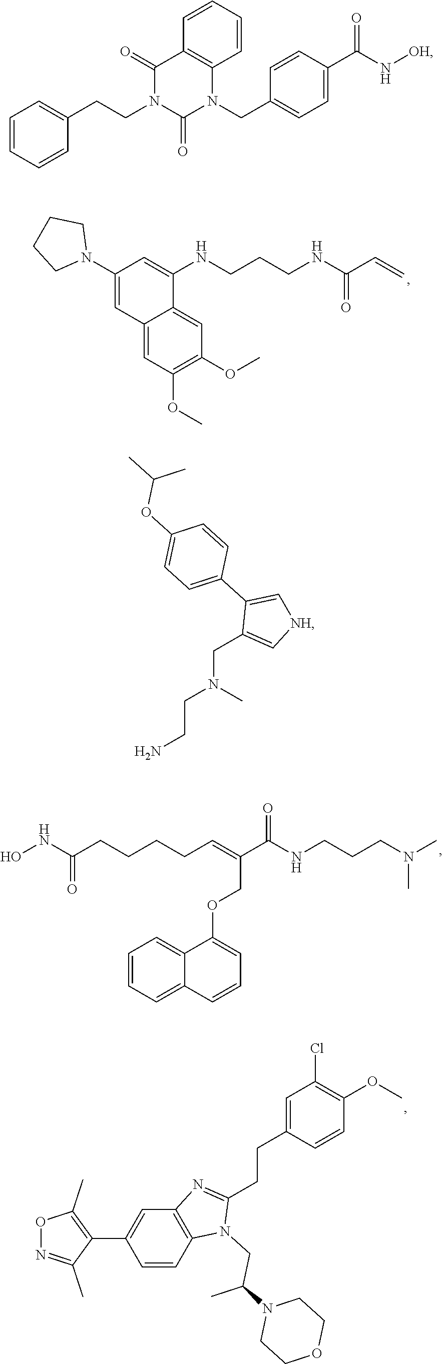

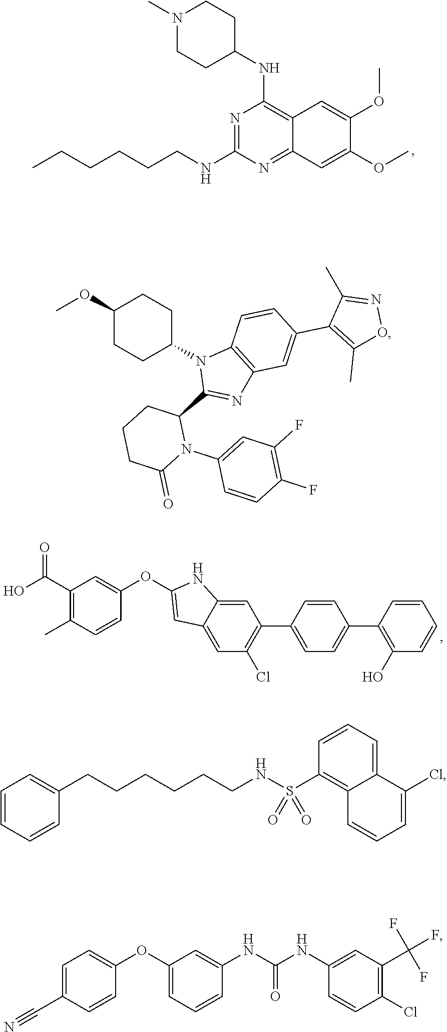

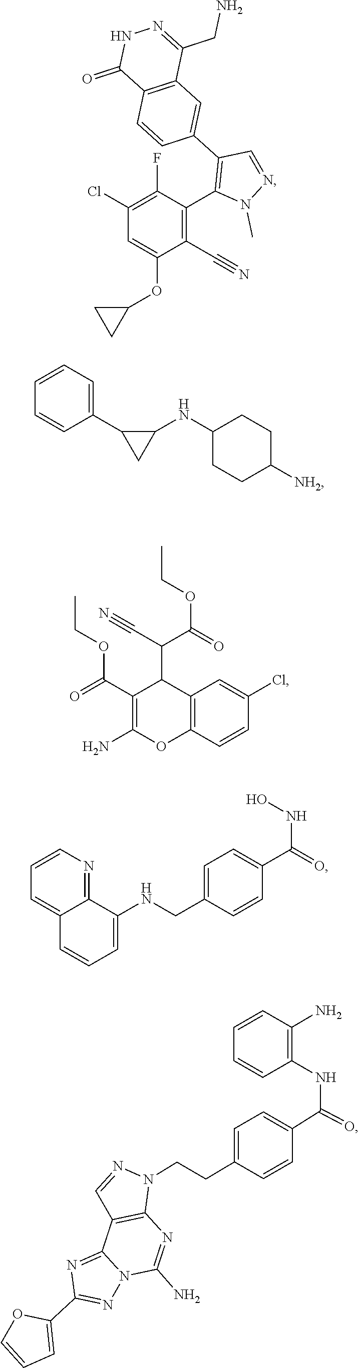 [SMILES: Cn1ncc(-c2ccc3c(=O)[nH]nc(CN)c3c2)c1-c1c(F)c(Cl)cc(OC2CC2)c1C#N ; NC1CCC(NC2CC2c2ccccc2)CC1 ; CCOC(=O)C1=C(N)Oc2ccc(Cl)cc2C1C(C#N)C(=O)OCC ; O=C(NO)c1ccc(CNc2cccc3cccnc23)cc1 ; Nc1ccccc1NC(=O)c1ccc(CCn2ncc3c2nc(N)n2nc(-c4ccco4)nc32)cc1]

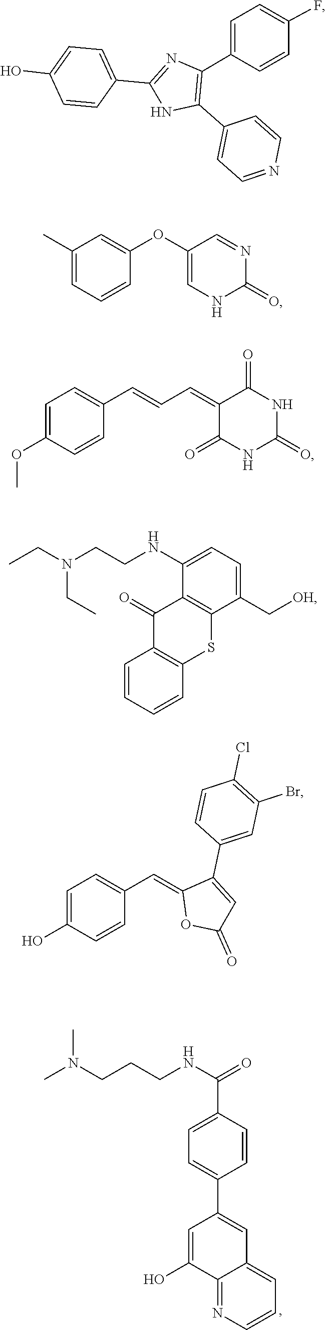 [SMILES: Oc1ccc(-c2nc(-c3ccc(F)cc3)c(-c3ccncc3)[nH]2)cc1 ; Cc1cccc(Oc2cnc(=O)[nH]c2)c1 ; COc1ccc(/C=C/C=C2C(=O)NC(=O)NC2=O)cc1 ; CCN(CC)CCNc1ccc(CO)c2sc3ccccc3c(=O)c12 ; O=C1C=C(c2ccc(Cl)c(Br)c2)/C(=C/c2ccc(O)cc2)O1 ; CN(C)CCCNC(=O)c1ccc(-c2cc(O)c3ncccc3c2)cc1]

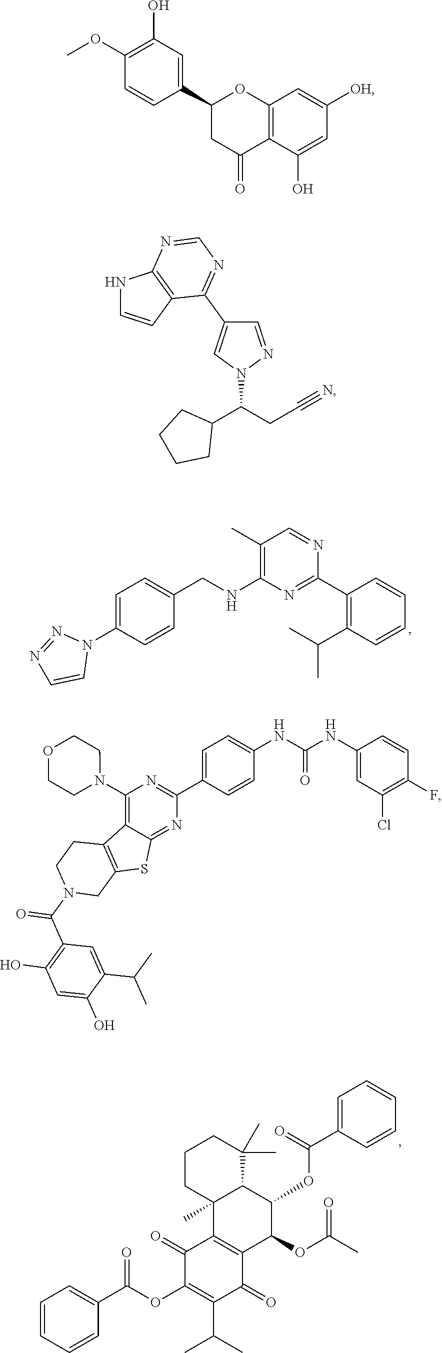 [SMILES: COc1ccc([C@@H]2CC(=O)c3c(O)cc(O)cc3O2)cc1O ; N#CC[C@H](C1CCCC1)n1cc(-c2ncnc3[nH]ccc23)cn1 ; Cc1cnc(-c2ccccc2C(C)C)nc1NCc1ccc(-n2ccnn2)cc1 ; CC(C)c1cc(C(=O)N2CCc3c(sc4nc(-c5ccc(NC(=O)Nc6ccc(F)c(Cl)c6)cc5)nc(N5CCOCC5)c34)C2)c(O)cc1O ; CC(=O)O[C@H]1C2=C(C(=O)C(OC(=O)c3ccccc3)=C(C(C)C)C2=O)[C@@]2(C)CCCC(C)(C)[C@@H]2[C@@H]1OC(=O)c1ccccc1]

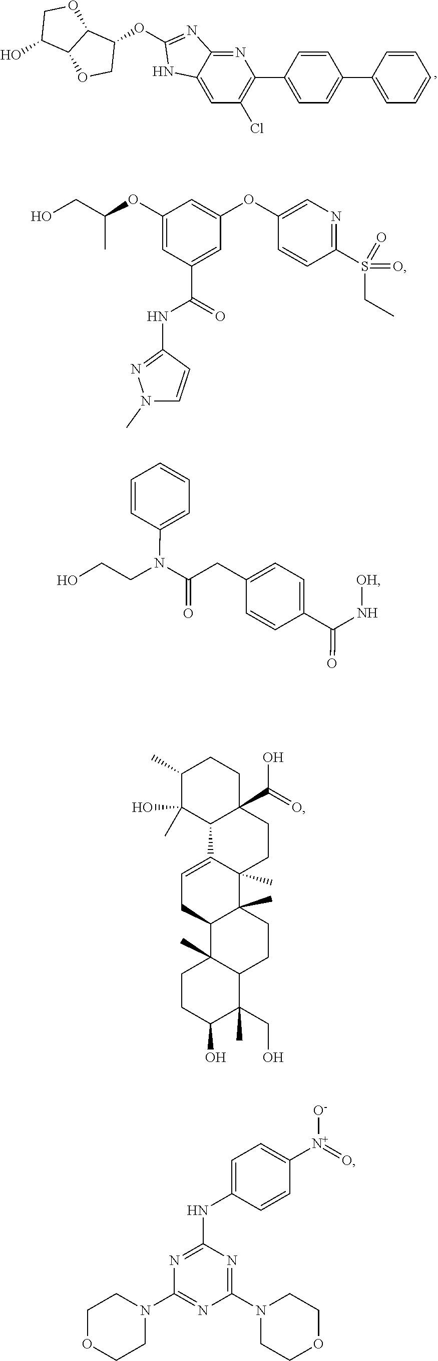 [SMILES: O[C@@H]1CO[C@H]2[C@@H]1OC[C@H]2Oc1nc2nc(-c3ccc(-c4ccccc4)cc3)c(Cl)cc2[nH]1 ; CCS(=O)(=O)c1ccc(Oc2cc(O[C@@H](C)CO)cc(C(=O)Nc3ccn(C)n3)c2)cn1 ; O=C(NO)c1ccc(CC(=O)N(CCO)c2ccccc2)cc1 ; C[C@@H]1CC[C@]2(C(=O)O)CC[C@]3(C)C(=CC[C@@H]4[C@@]5(C)CC[C@H](O)[C@@](C)(CO)C5CC[C@]43C)[C@@H]2[C@]1(C)O ; O=[N+]([O-])c1ccc(Nc2nc(N3CCOCC3)nc(N3CCOCC3)n2)cc1]

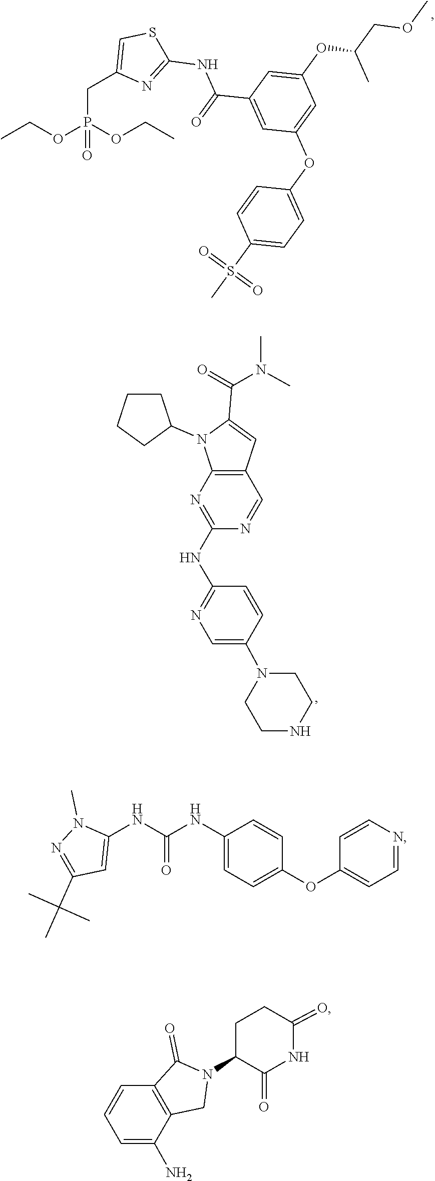

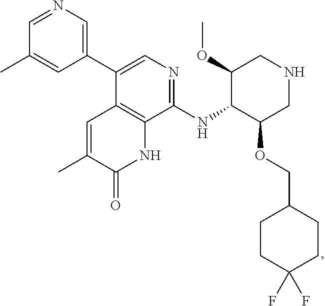 [SMILES: CO[C@H]1CNC[C@@H](OCC2CCC(F)(F)CC2)[C@@H]1Nc1ncc(-c2cncc(C)c2)c2cc(C)c(=O)[nH]c12]

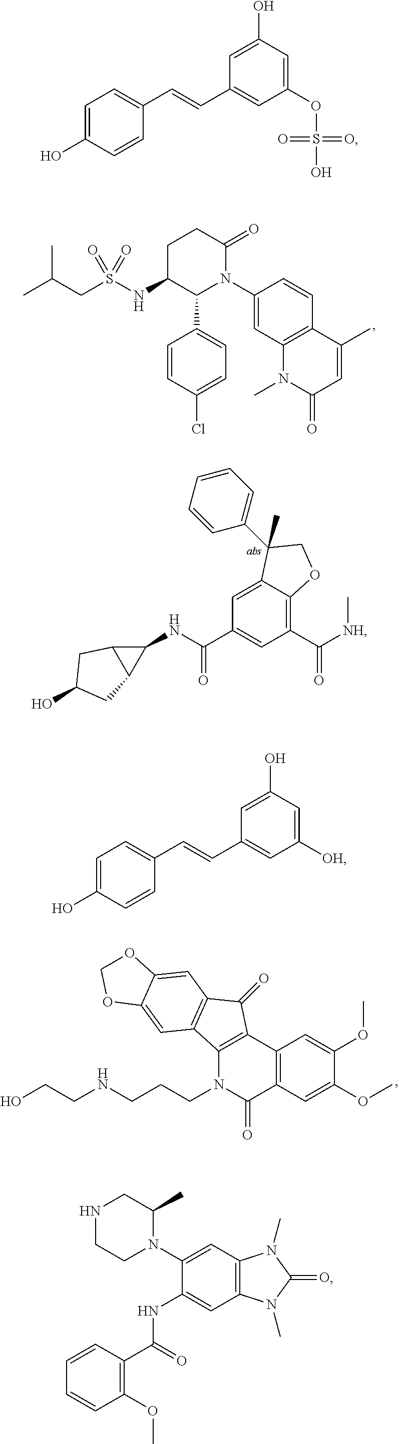

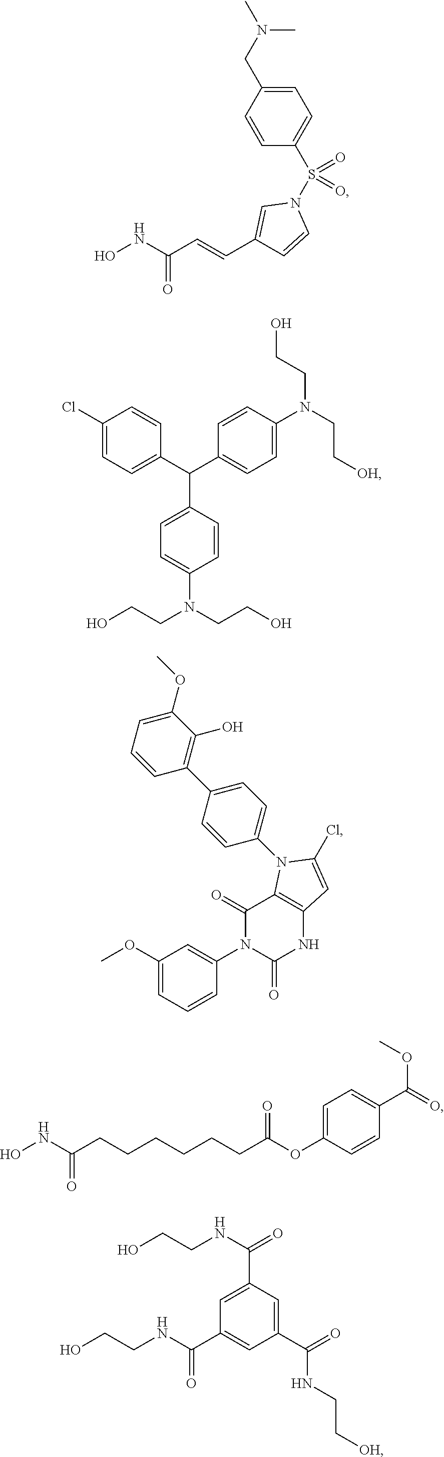

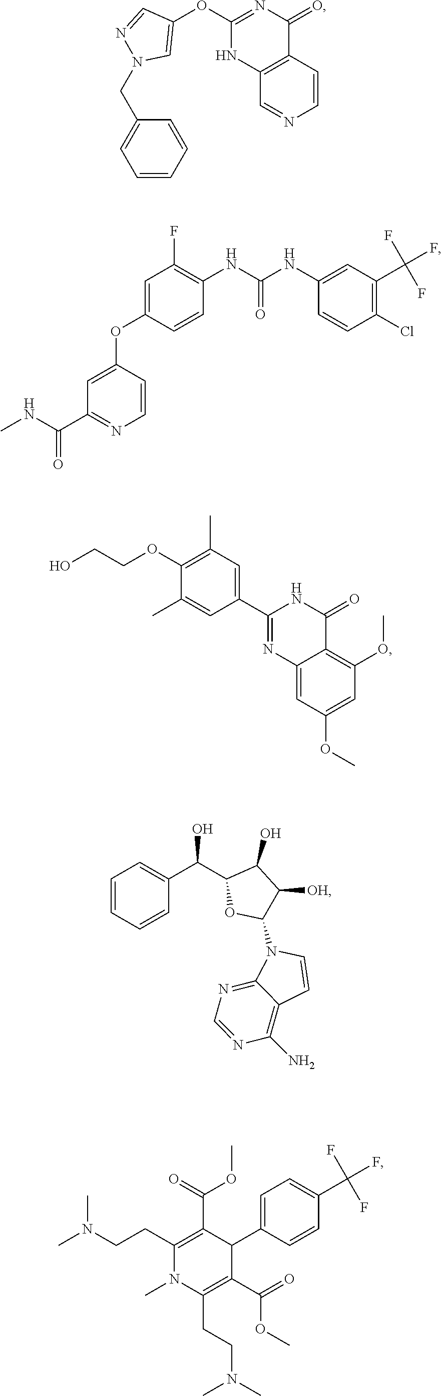

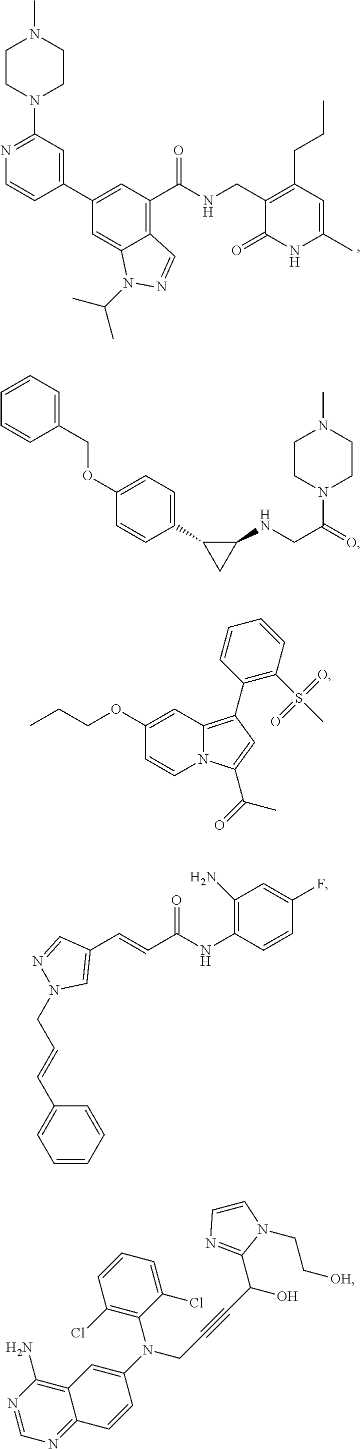

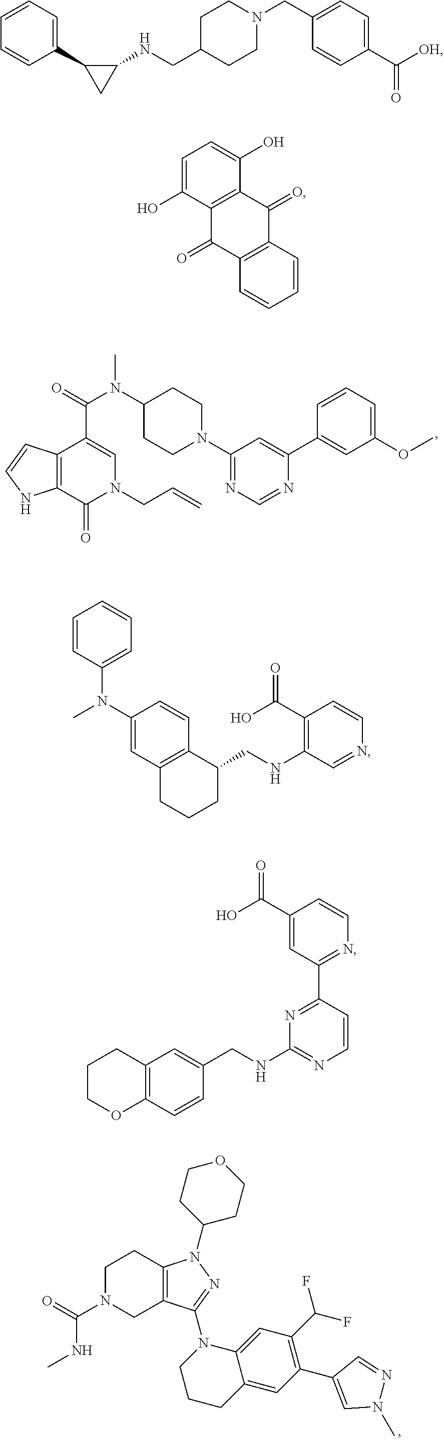 [SMILES: O=C(O)c1ccc(CN2CCC(CN[C@@H]3C[C@H]3c3ccccc3)CC2)cc1 ; O=C1c2ccccc2C(=O)c2c(O)ccc(O)c21 ; C=CCn1cc(C(=O)N(C)C2CCN(c3cc(-c4cccc(OC)c4)ncn3)CC2)c2cc[nH]c2c1=O ; CN(c1ccccc1)c1ccc2c(c1)CCC[C@H]2CNc1cnccc1C(=O)O ; O=C(O)c1ccnc(-c2ccnc(NCc3ccc4c(c3)CCCO4)n2)c1 ; CNC(=O)N1CCc2c(c(N3CCCc4cc(-c5cnn(C)c5)c(C(F)F)cc43)nn2C2CCOCC2)C1]

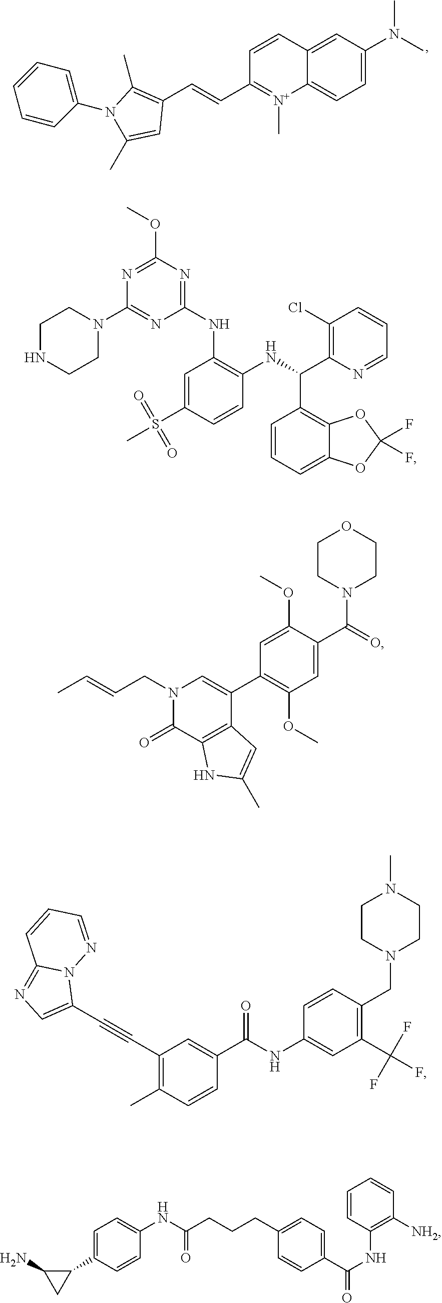

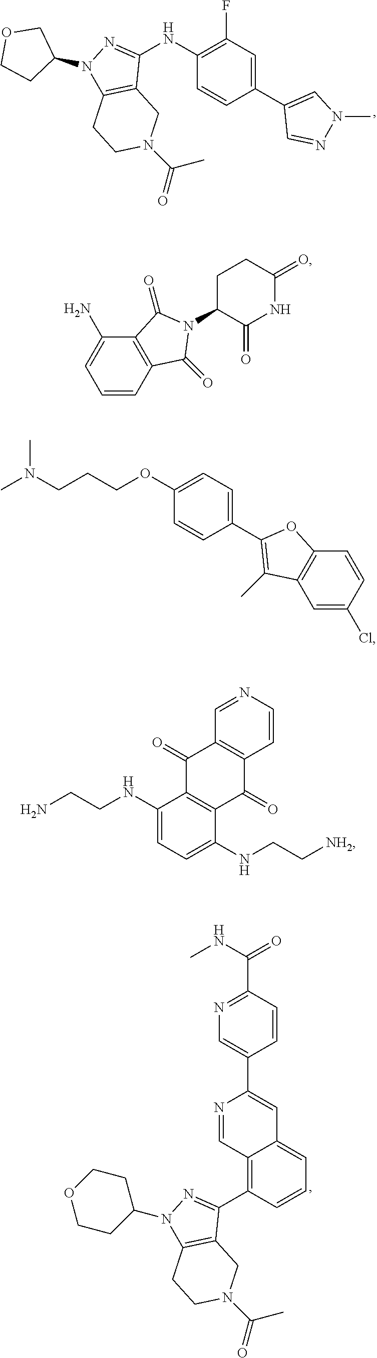

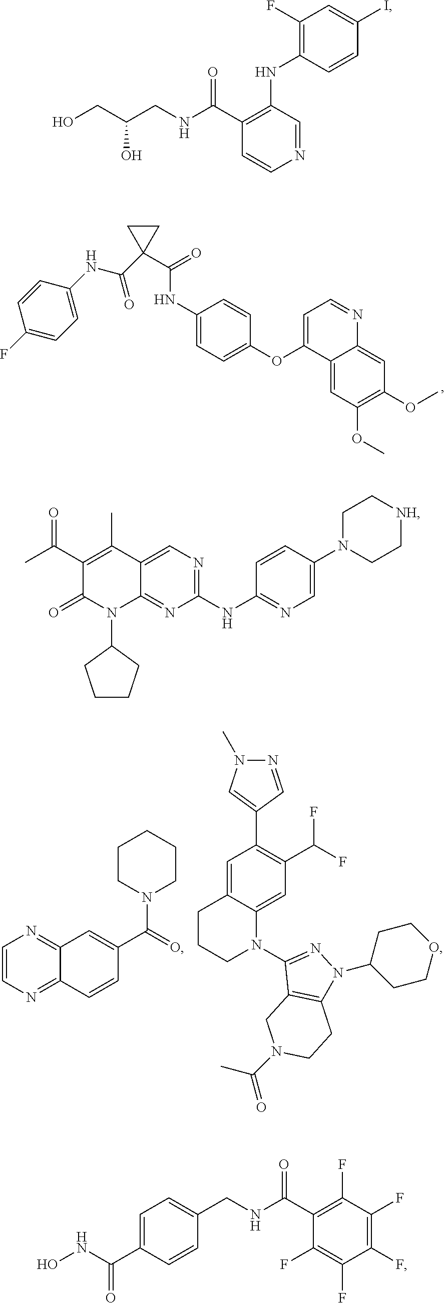

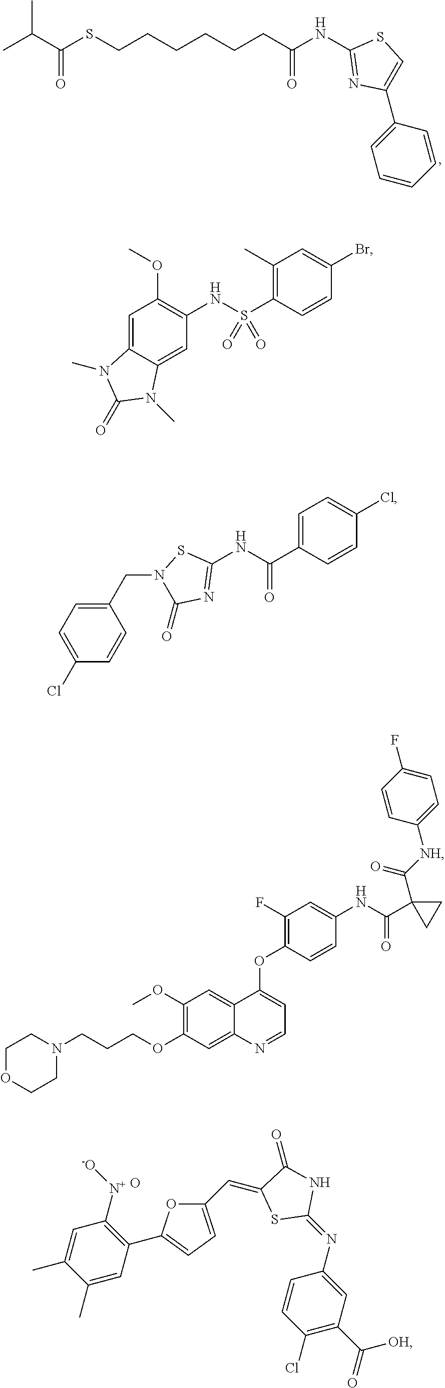

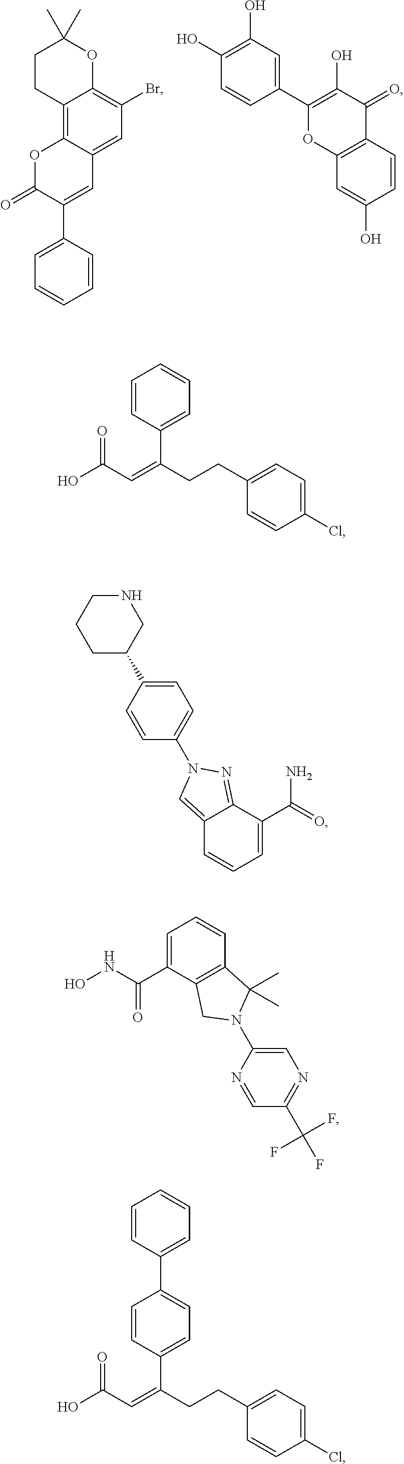 [SMILES: CC1(C)CCc2c(c(Br)cc3cc(-c4ccccc4)c(=O)oc23)O1 ; O=c1c(O)c(-c2ccc(O)c(O)c2)oc2cc(O)ccc12 ; O=C(O)/C=C(/CCc1ccc(Cl)cc1)c1ccccc1 ; NC(=O)c1cccc2cn(-c3ccc([C@@H]4CCCNC4)cc3)nc12 ; CC1(C)c2cccc(C(=O)NO)c2CN1c1cnc(C(F)(F)F)cn1 ; O=C(O)/C=C(/CCc1ccc(Cl)cc1)c1ccc(-c2ccccc2)cc1]

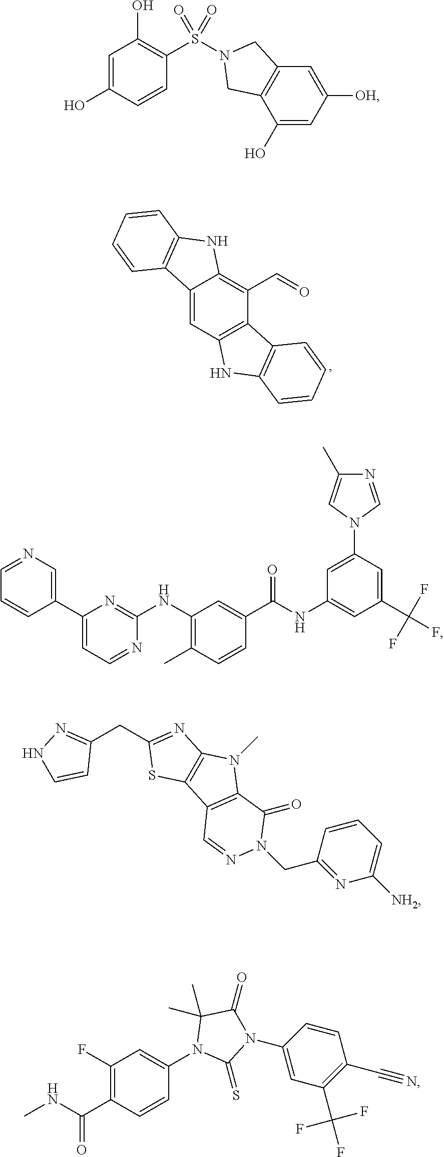 [SMILES: O=S(=O)(c1ccc(O)cc1O)N1Cc2cc(O)cc(O)c2C1 ; O=Cc1c2[nH]c3ccccc3c2cc2[nH]c3ccccc3c12 ; Cc1cn(-c2cc(NC(=O)c3ccc(C)c(Nc4nccc(-c5cccnc5)n4)c3)cc(C(F)(F)F)c2)cn1 ; Cn1c2nc(Cc3cc[nH]n3)sc2c2cnn(Cc3cccc(N)n3)c(=O)c21 ; CNC(=O)c1ccc(N2C(=S)N(c3ccc(C#N)c(C(F)(F)F)c3)C(=O)C2(C)C)cc1F]

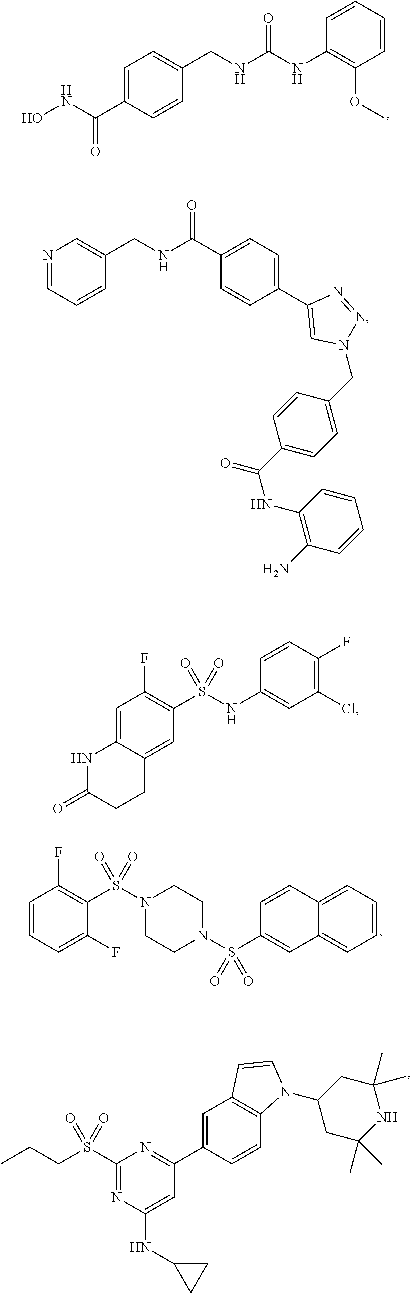

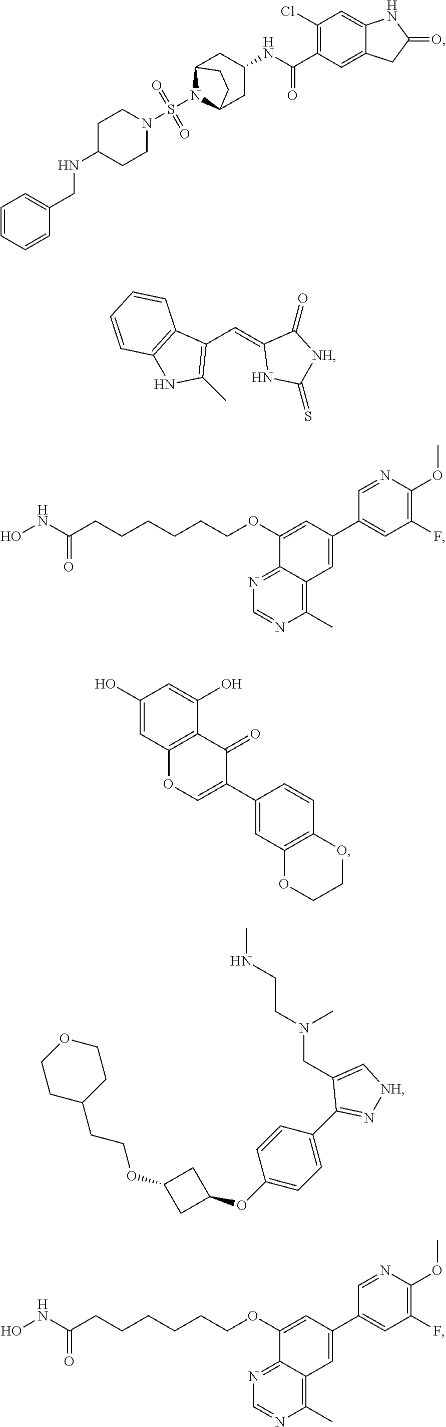 [SMILES: O=C1Cc2cc(C(=O)N[C@H]3C[C@H]4CC[C@@H](C3)N4S(=O)(=O)N3CCC(NCc4ccccc4)CC3)c(Cl)cc2N1 ; Cc1[nH]c2ccccc2c1/C=C1\NC(=S)NC1=O ; COc1ncc(-c2cc(OCCCCCCC(=O)NO)c3ncnc(C)c3c2)cc1F ; O=c1c(-c2ccc3c(c2)OCCO3)coc2cc(O)cc(O)c12 ; CNCCN(C)Cc1c[nH]nc1-c1ccc(O[C@H]2C[C@H](OCCC3CCOCC3)C2)cc1 ; COc1ncc(-c2cc(OCCCCCCC(=O)NO)c3ncnc(C)c3c2)cc1F]

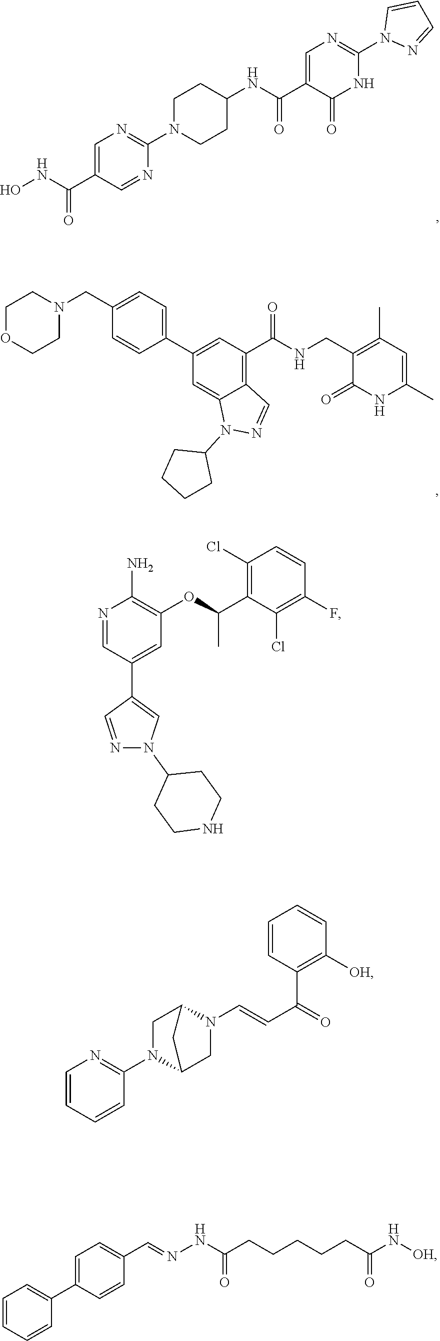 [SMILES: O=C(NO)c1cnc(N2CCC(NC(=O)c3cnc(-n4cccn4)[nH]c3=O)CC2)nc1 ; Cc1cc(C)c(CNC(=O)c2cc(-c3ccc(CN4CCOCC4)cc3)cc3c2cnn3C2CCCC2)c(=O)[nH]1 ; C[C@@H](Oc1cc(-c2cnn(C3CCNCC3)c2)cnc1N)c1c(Cl)ccc(F)c1Cl ; O=C(/C=C/N1C[C@H]2C[C@@H]1CN2c1ccccn1)c1ccccc1O ; O=C(CCCCCC(=O)N/N=C/c1ccc(-c2ccccc2)cc1)NO]

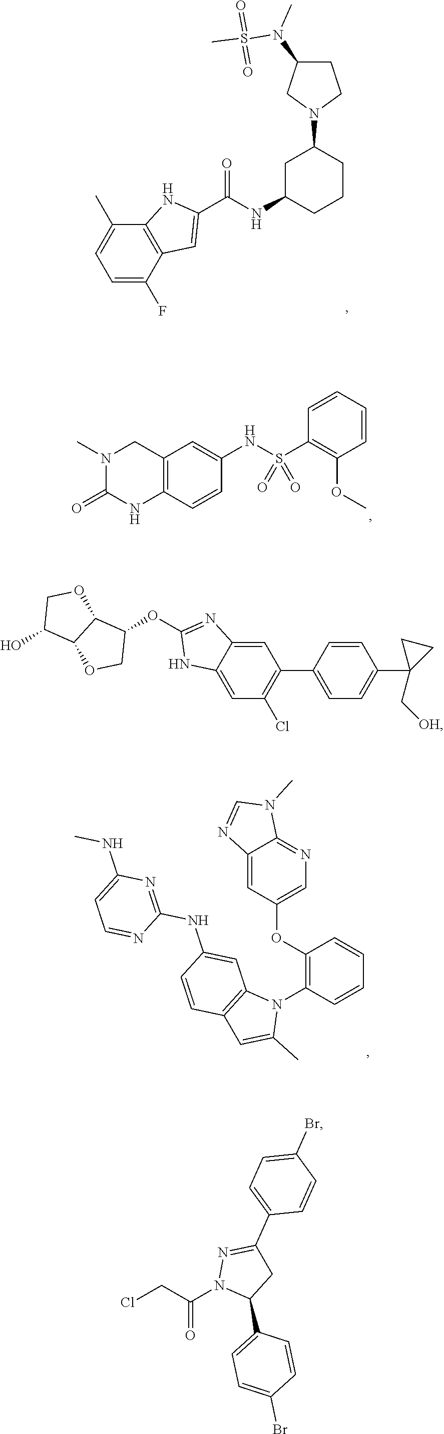 [SMILES: Cc1ccc(F)c2cc(C(=O)N[C@@H]3CCC[C@H](N4CC[C@H](N(C)S(C)(=O)=O)C4)C3)[nH]c12 ; COc1ccccc1S(=O)(=O)Nc1ccc2c(c1)CN(C)C(=O)N2 ; OCC1(c2ccc(-c3cc4nc(O[C@@H]5CO[C@H]6[C@@H]5OC[C@H]6O)[nH]c4cc3Cl)cc2)CC1 ; CNc1ccnc(Nc2ccc3cc(C)n(-c4ccccc4Oc4cnc5c(c4)ncn5C)c3c2)n1 ; O=C(CCl)N1N=C(c2ccc(Br)cc2)C[C@H]1c1ccc(Br)cc1]

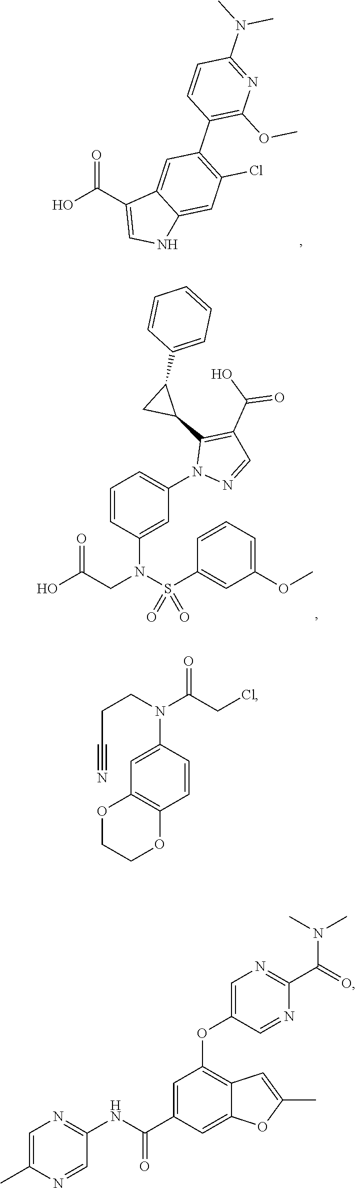

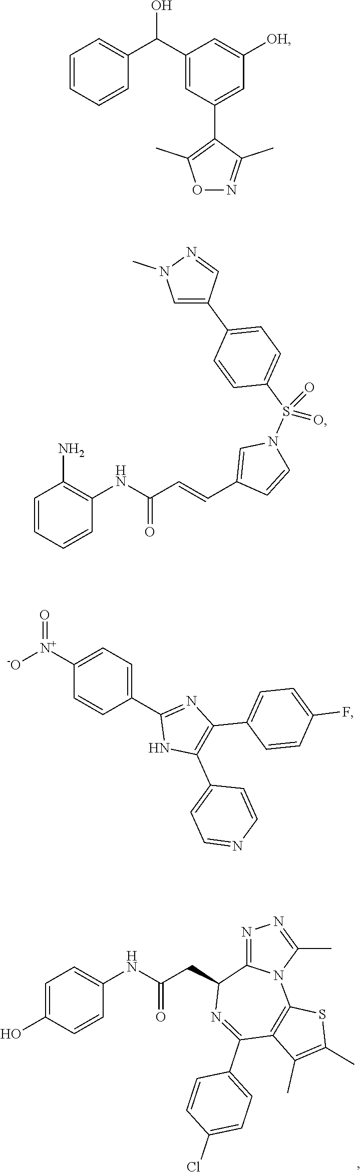

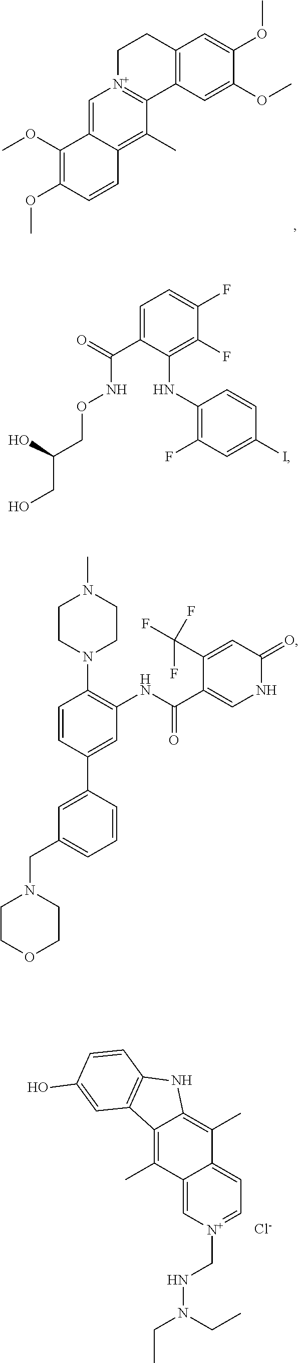

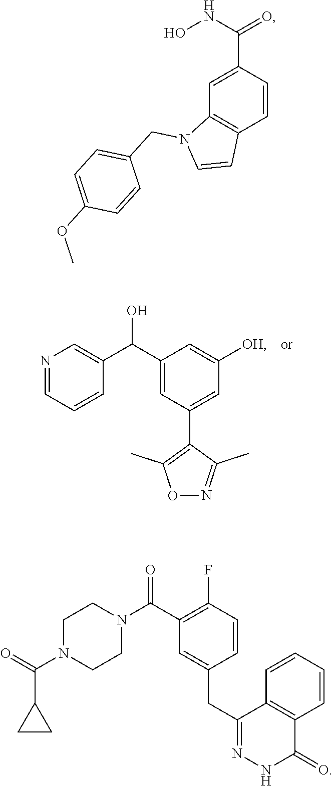 [SMILES: COc1ccc(Cn2ccc3ccc(C(=O)NO)cc32)cc1 ; Cc1noc(C)c1-c1cc(O)cc(C(O)c2cccnc2)c1 ; O=C(c1cc(Cc2n[nH]c(=O)c3ccccc23)ccc1F)N1CCN(C(=O)C2CC2)CC1]

In some embodiments, the parent ligand is

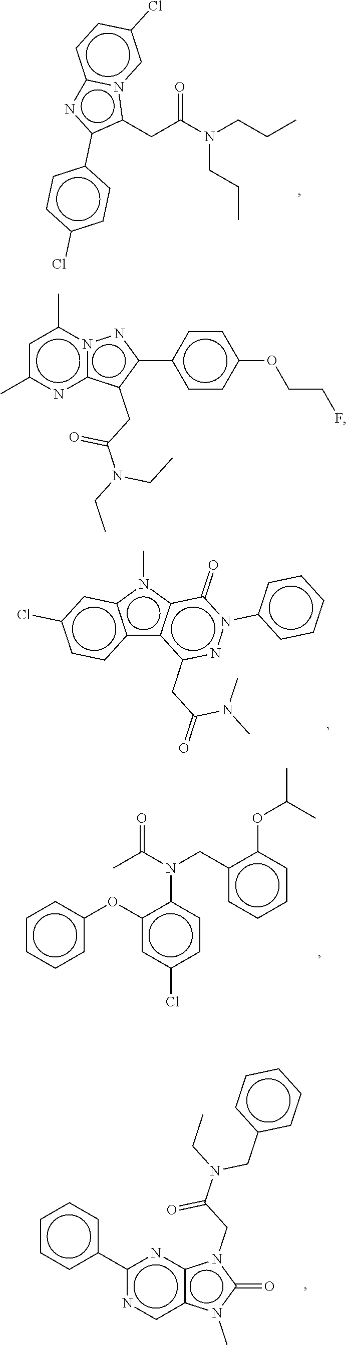

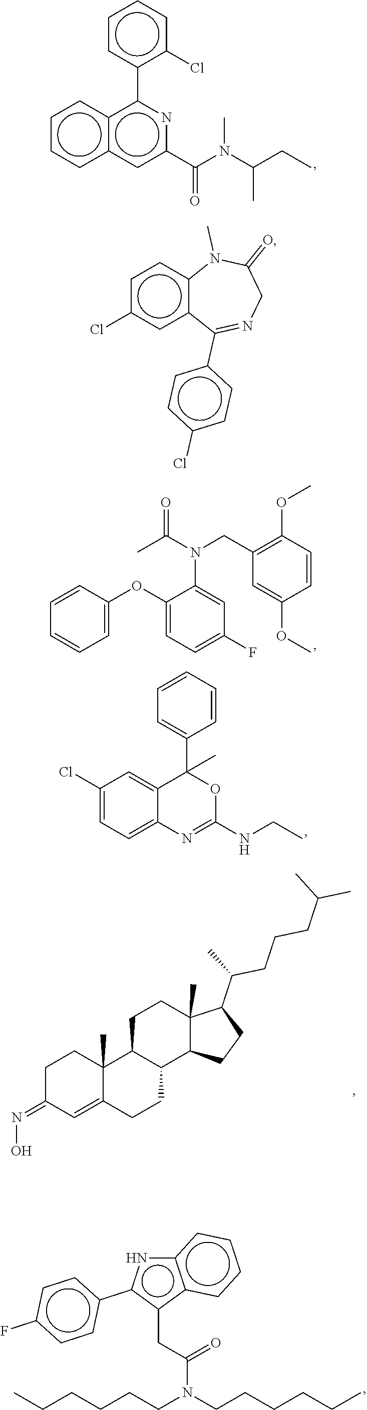

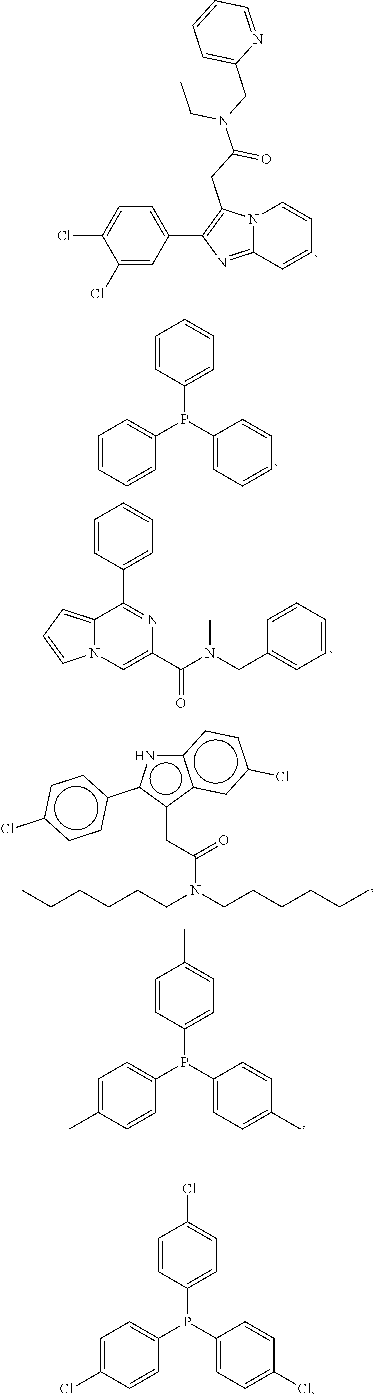

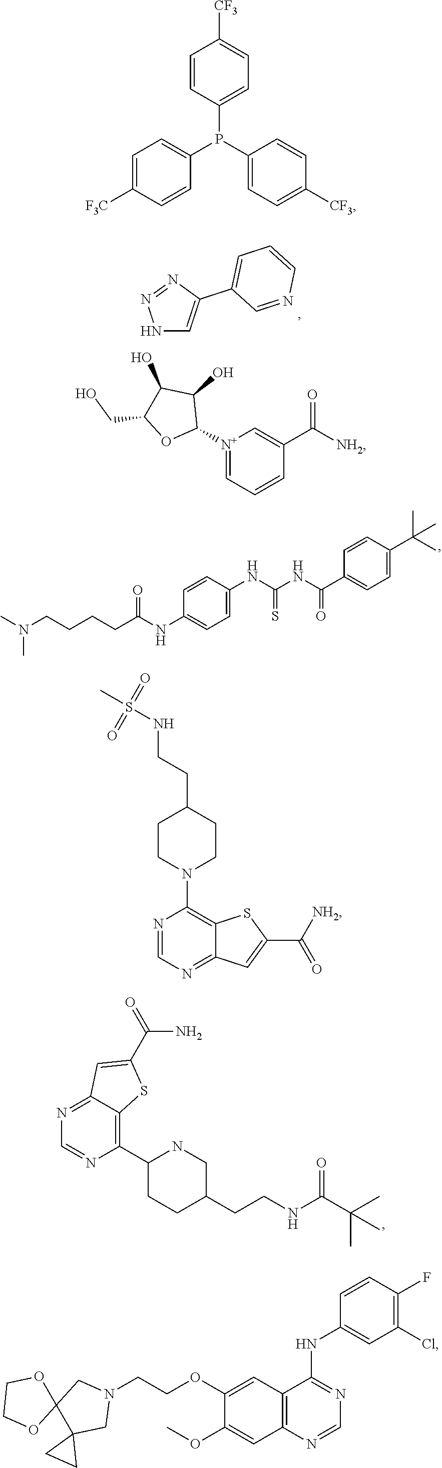

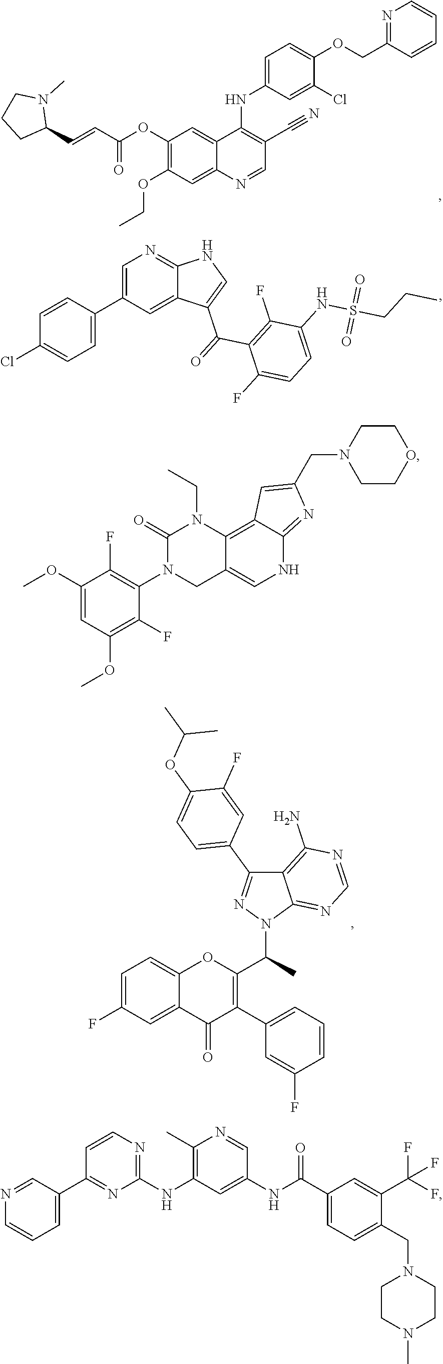

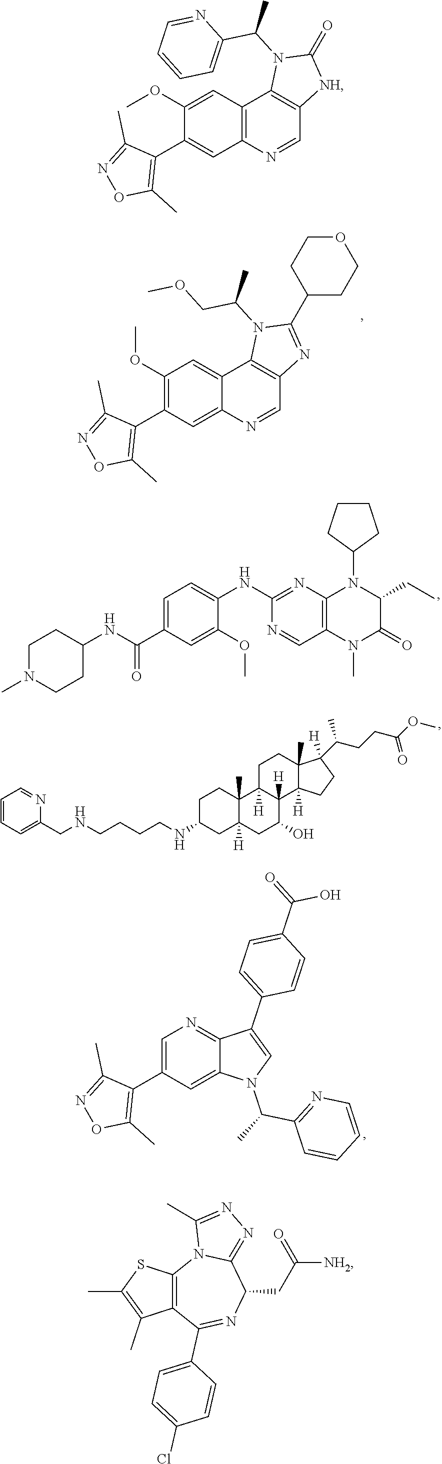

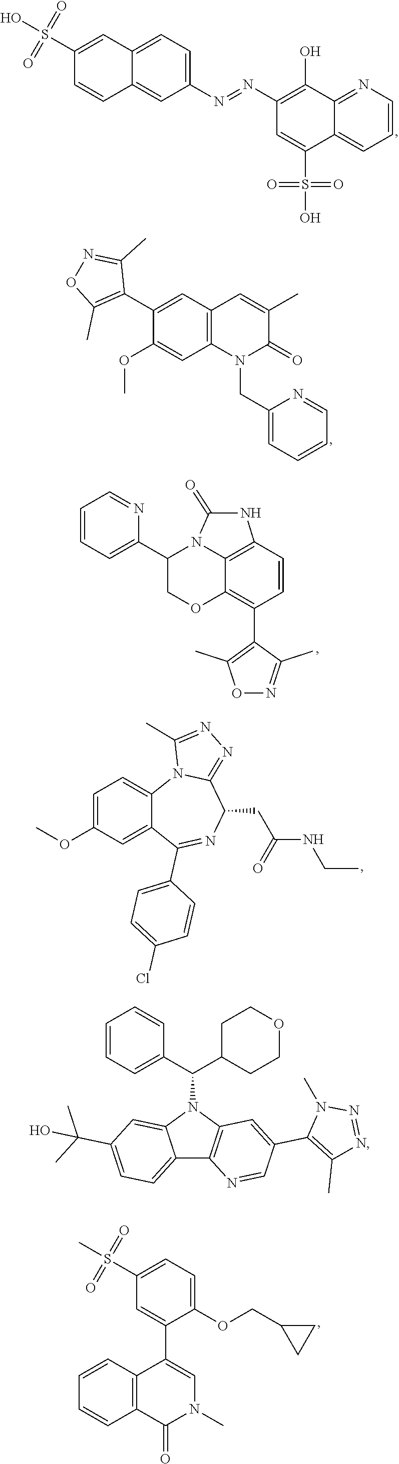

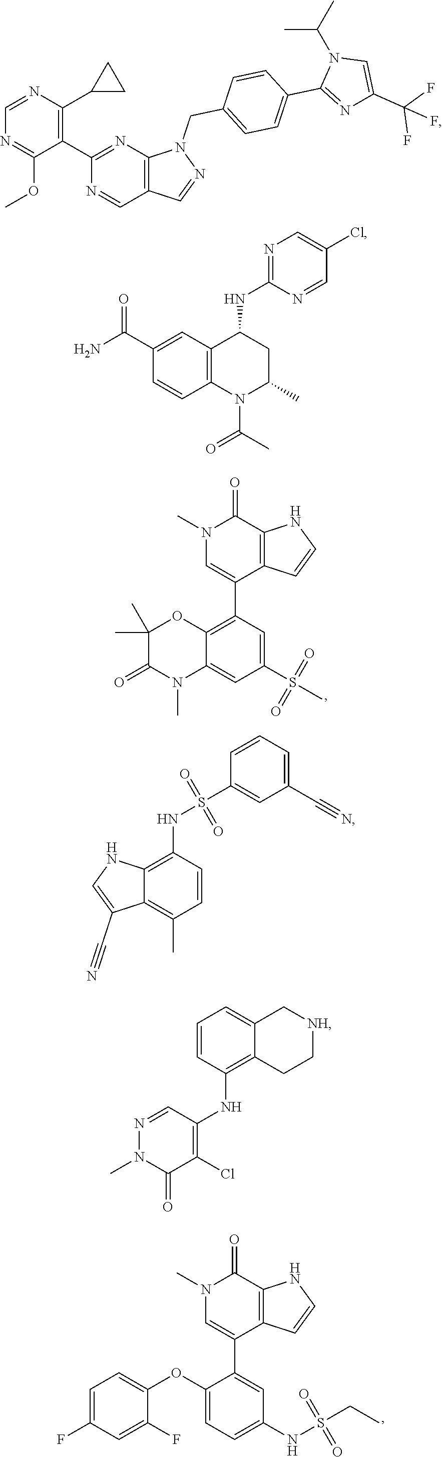

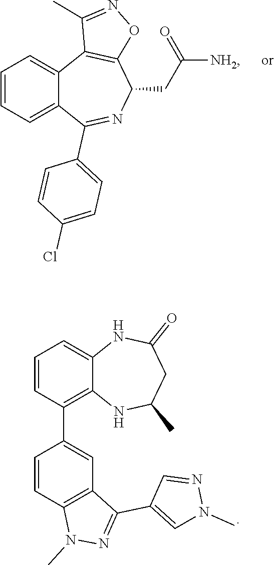 [SMILES: Cc1noc2c1-c1ccccc1C(c1ccc(Cl)cc1)=N[C@H]2CC(N)=O ; C[C@@H]1CC(=O)Nc2cccc(-c3ccc4c(c3)c(-c3cnn(C)c3)nn4C)c2N1]

In some embodiments, the parent ligand is:

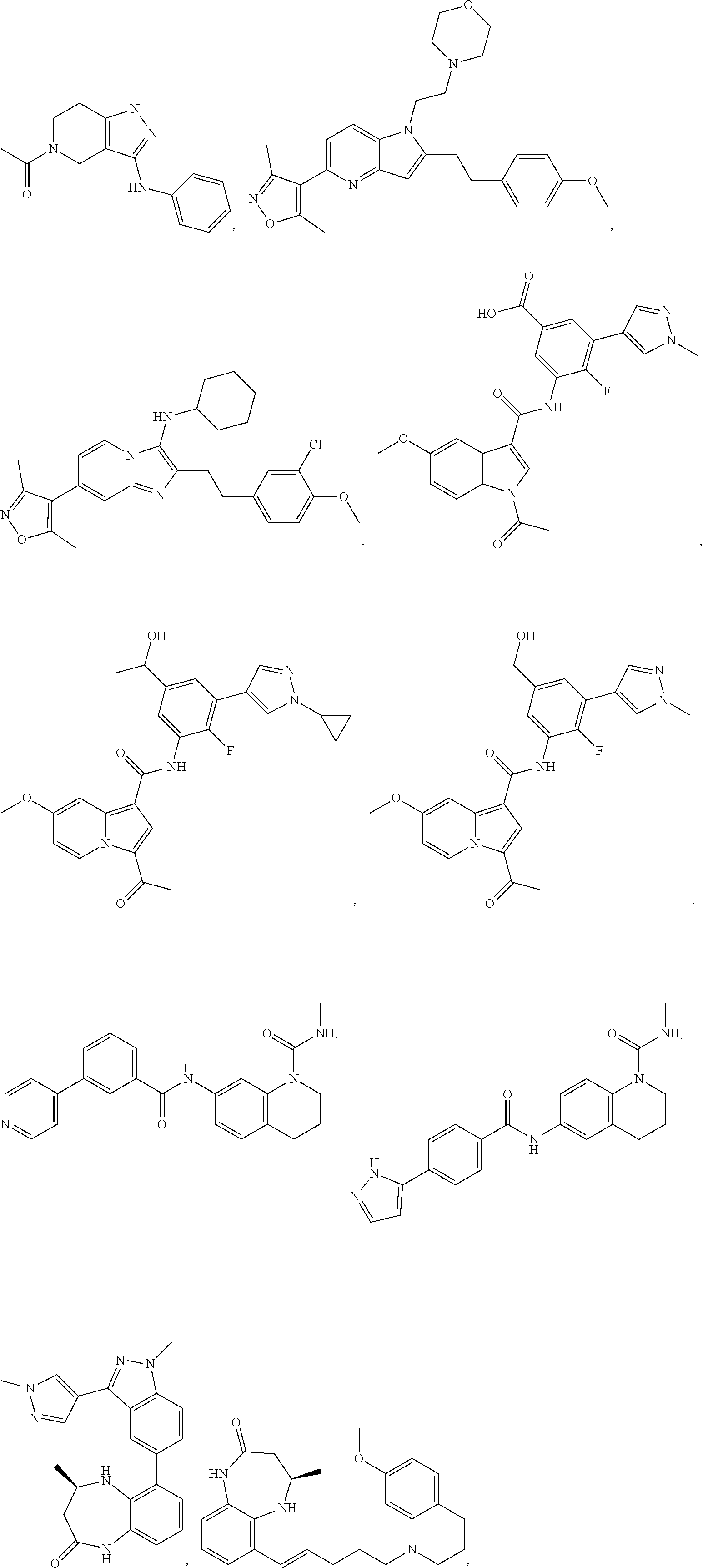

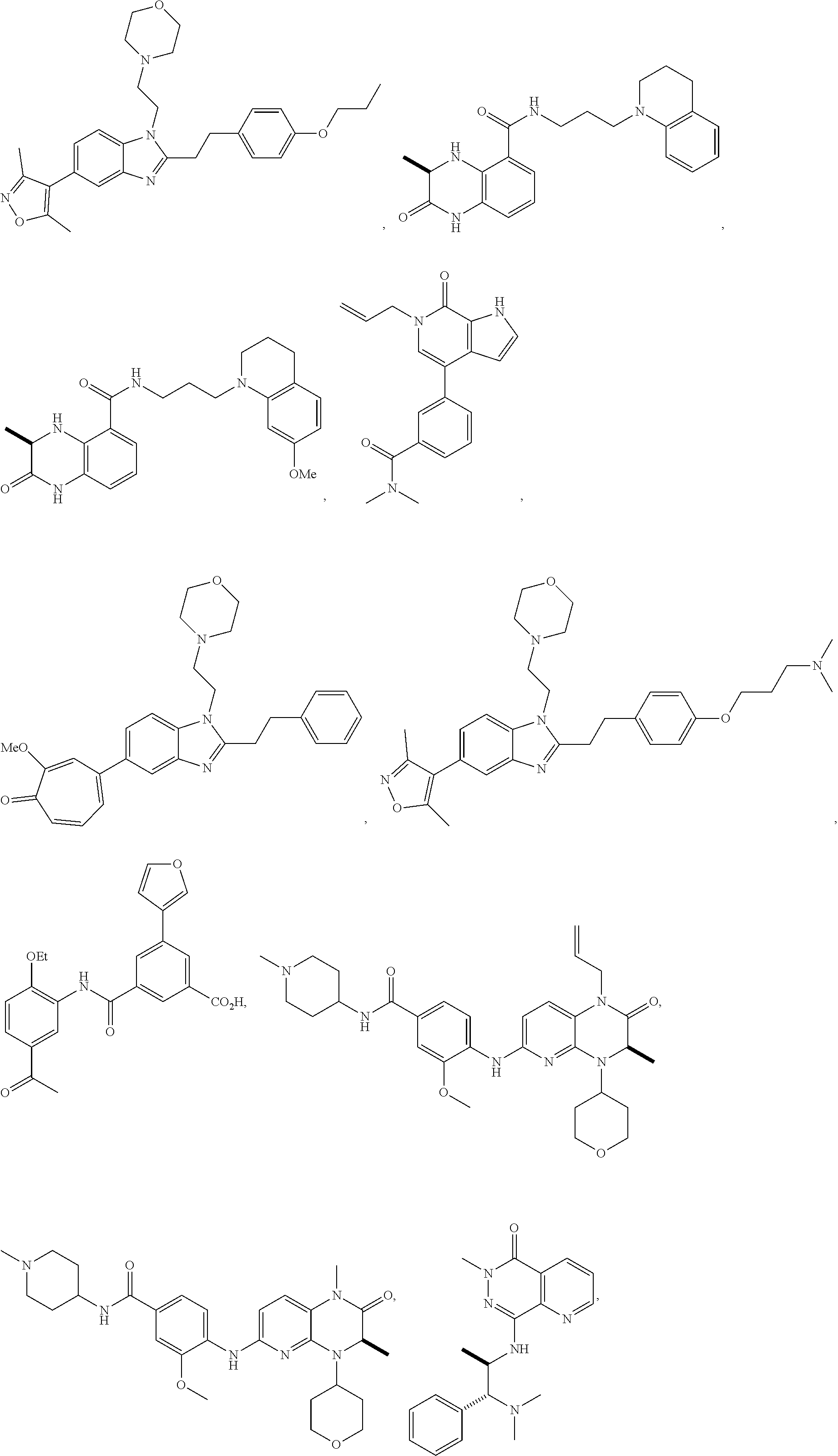

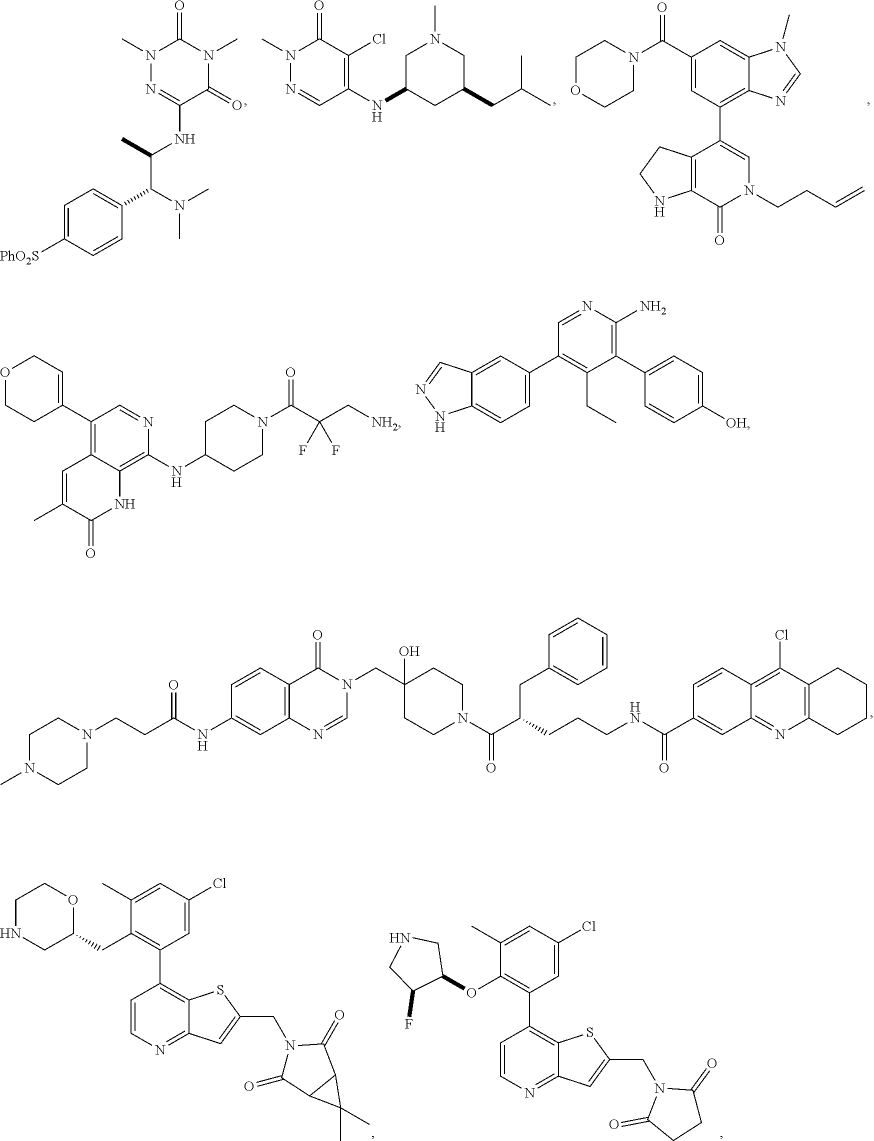

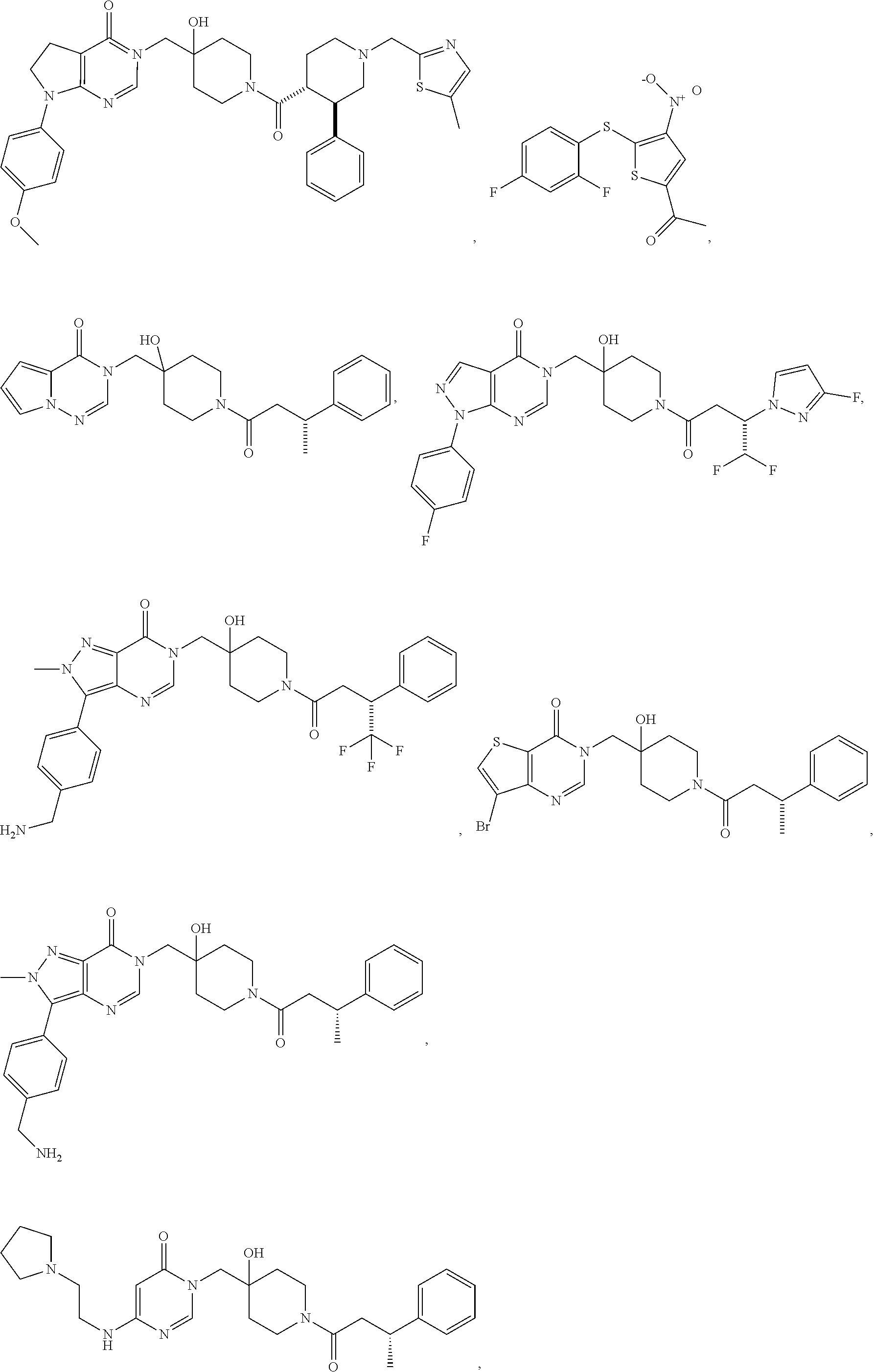

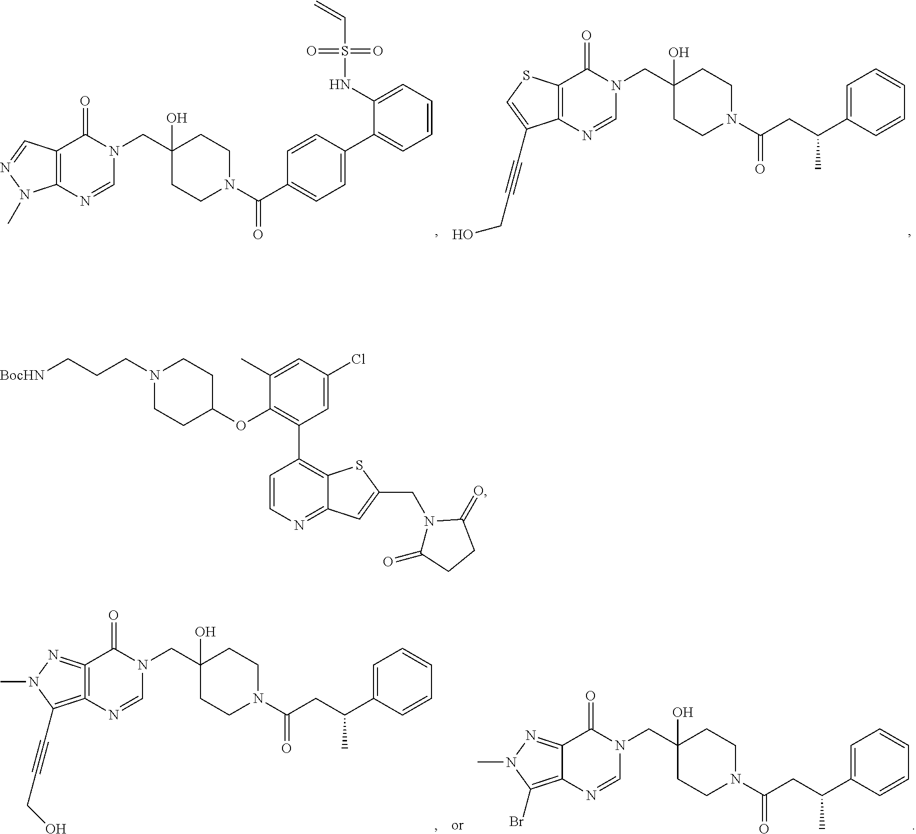

In some embodiments, the parent ligand is:

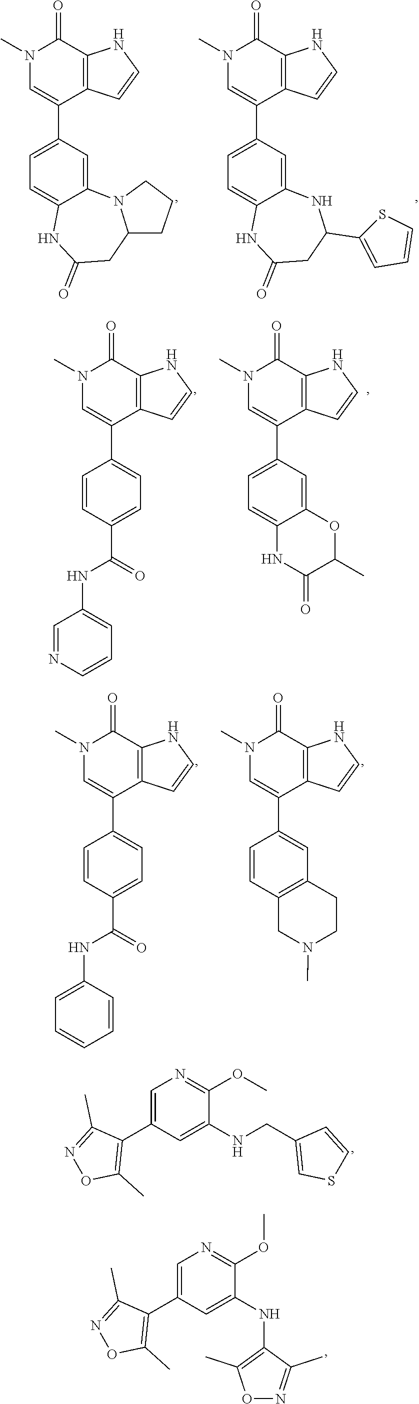

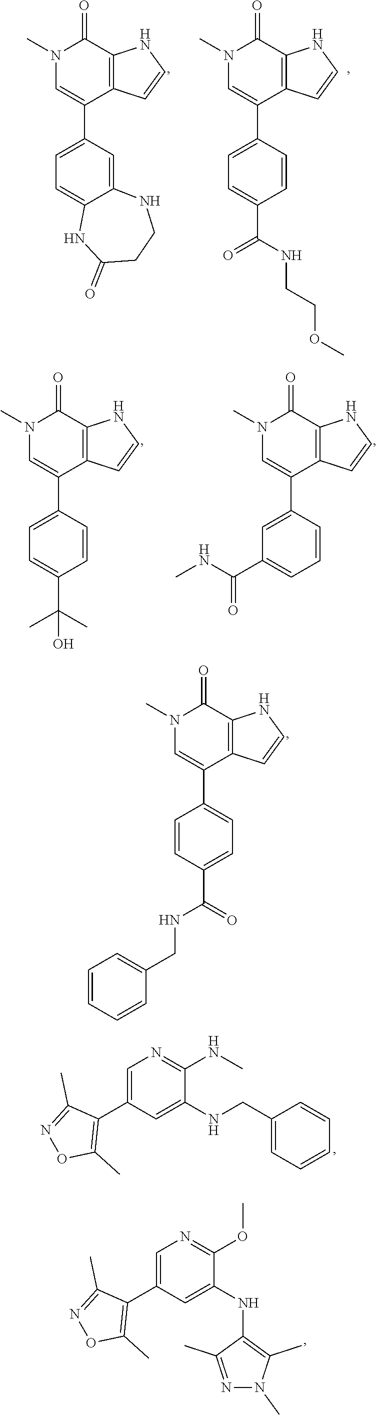

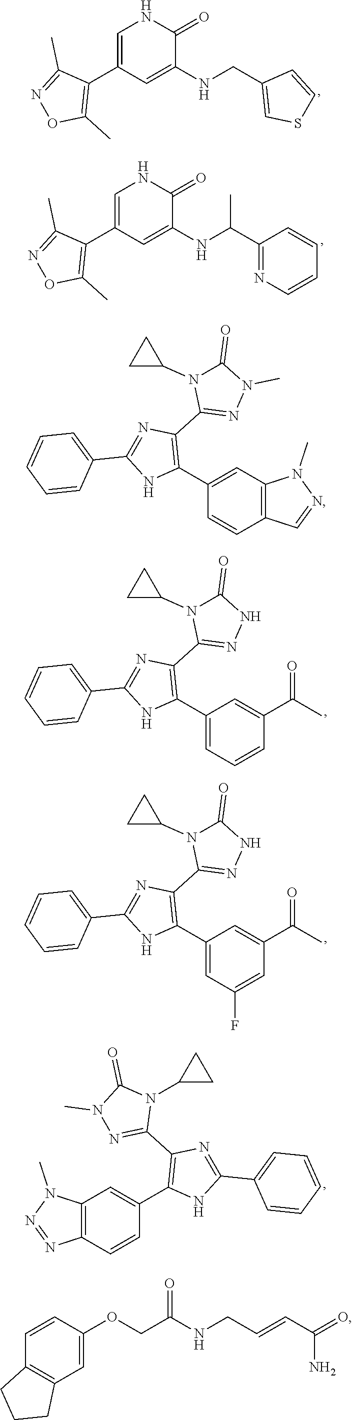

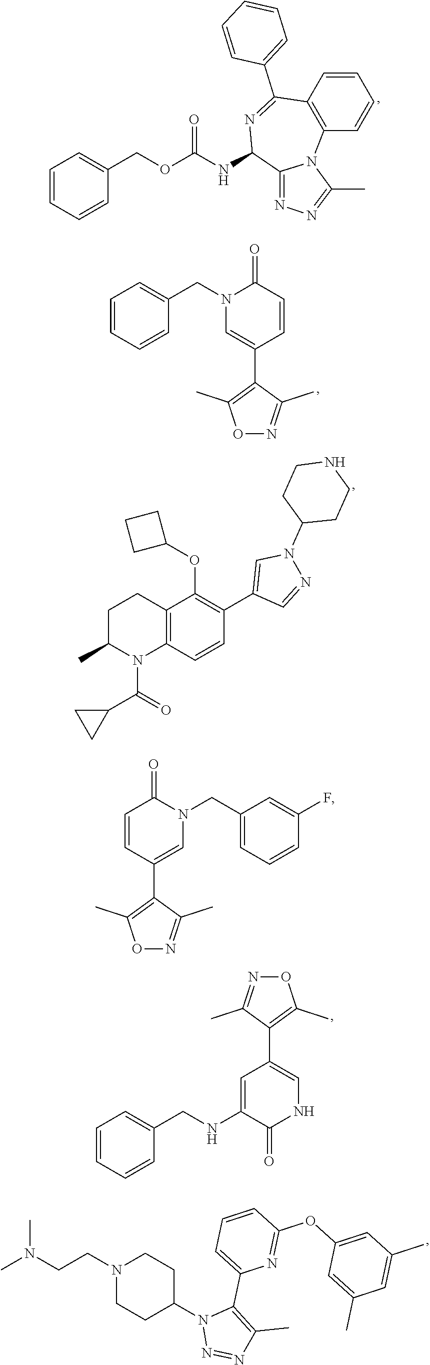

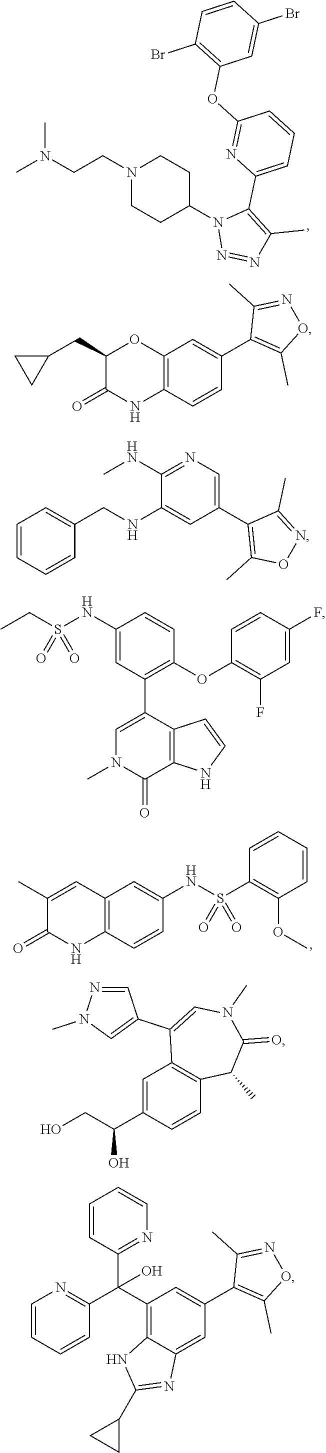

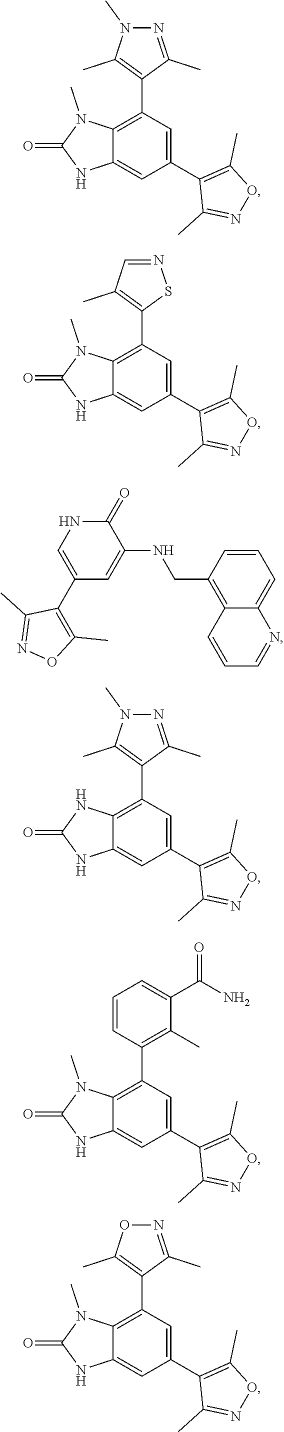

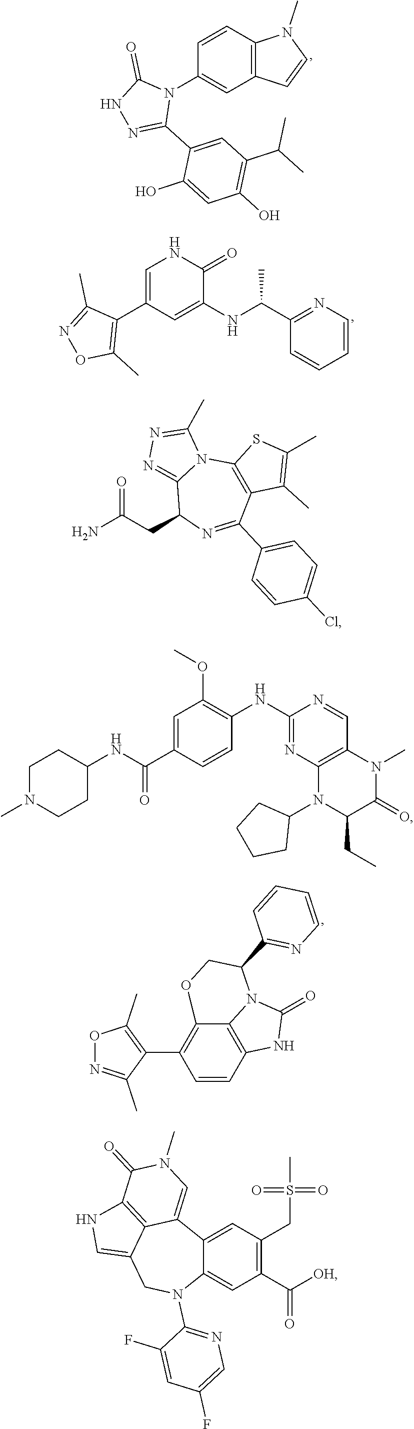

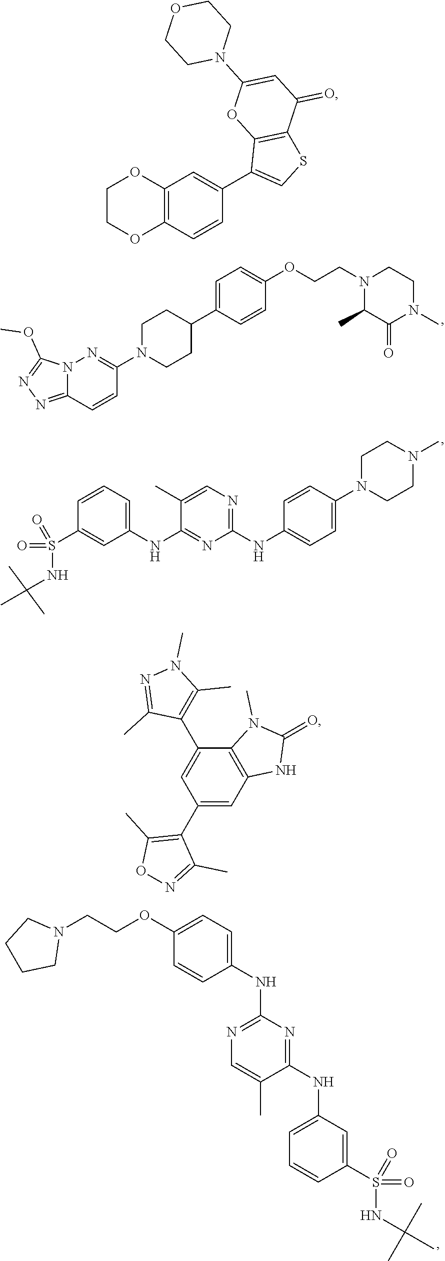

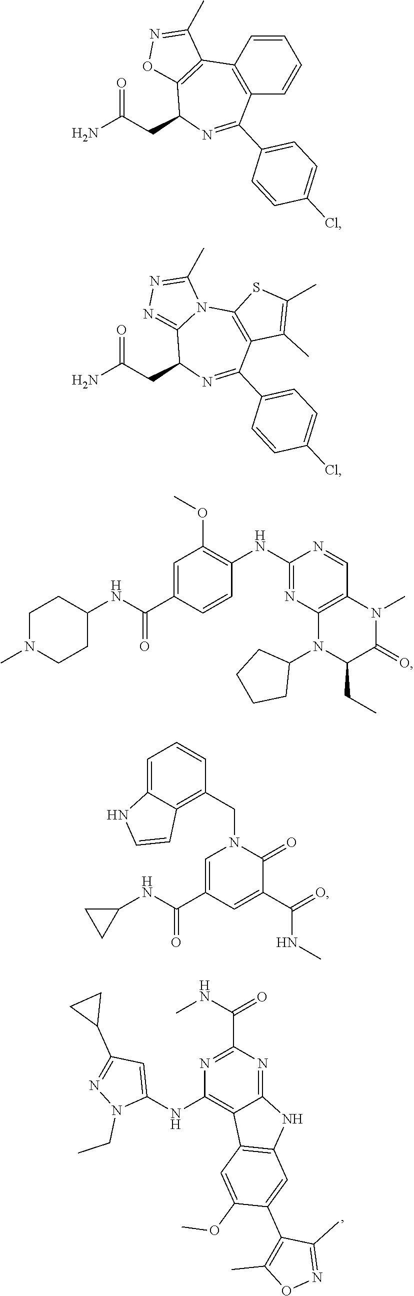 [SMILES: Cc1noc2c1-c1ccccc1C(c1ccc(Cl)cc1)=N[C@H]2CC(N)=O ; Cc1sc2c(c1C)C(c1ccc(Cl)cc1)=N[C@@H](CC(N)=O)c1nnc(C)n1-2 ; CC[C@@H]1C(=O)N(C)c2cnc(Nc3ccc(C(=O)NC4CCN(C)CC4)cc3OC)nc2N1C1CCCC1 ; CNC(=O)c1cc(C(=O)NC2CC2)cn(Cc2cccc3[nH]ccc23)c1=O ; CCn1nc(C2CC2)cc1Nc1nc(C(=O)NC)nc2[nH]c3cc(-c4c(C)noc4C)c(OC)cc3c12]

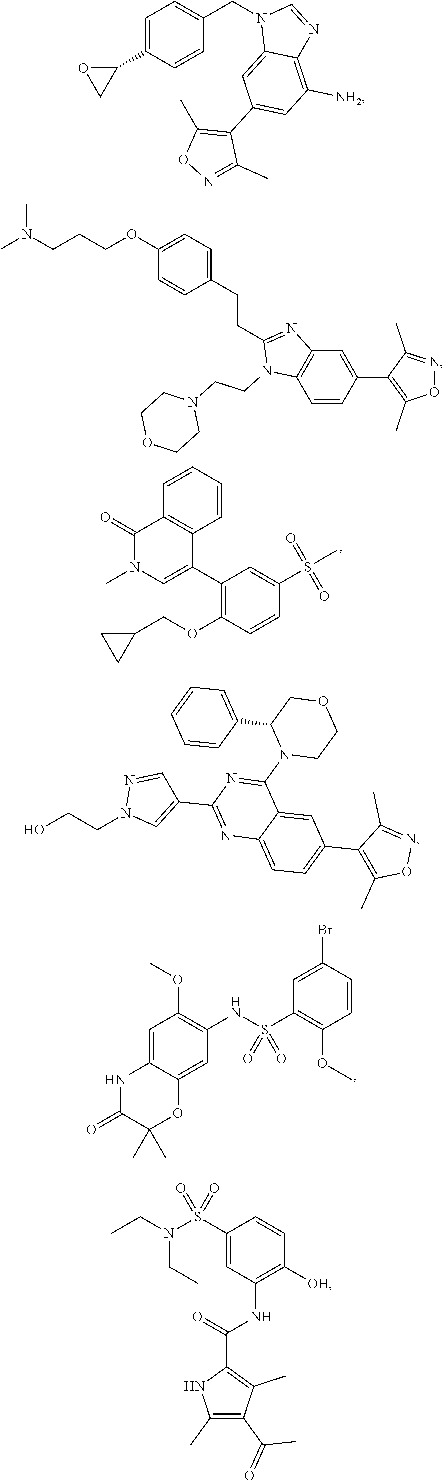

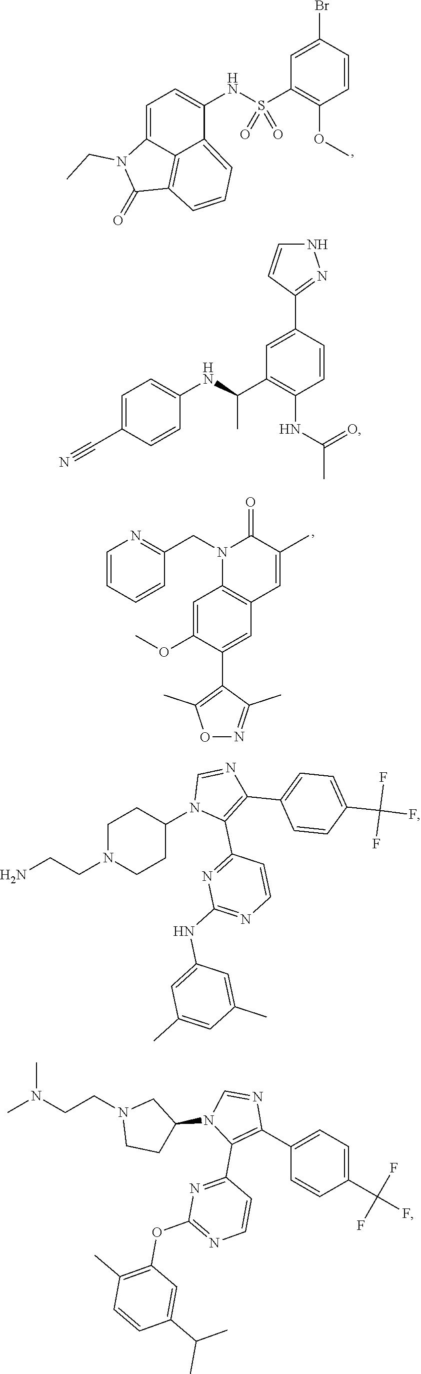

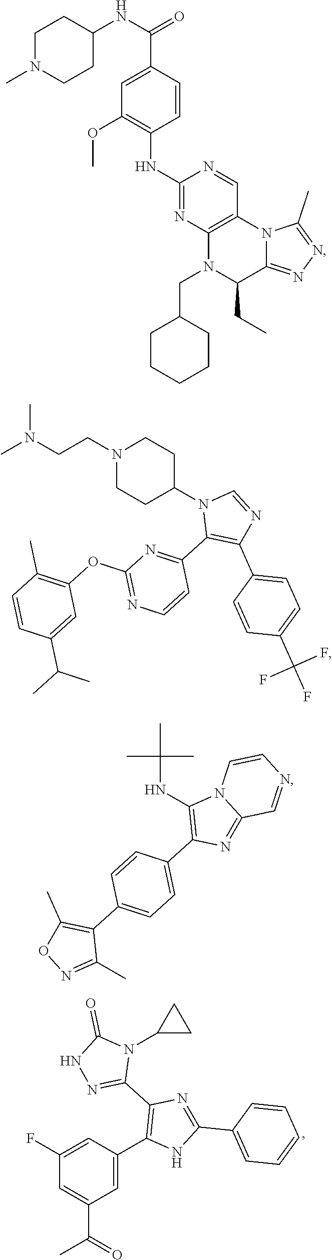

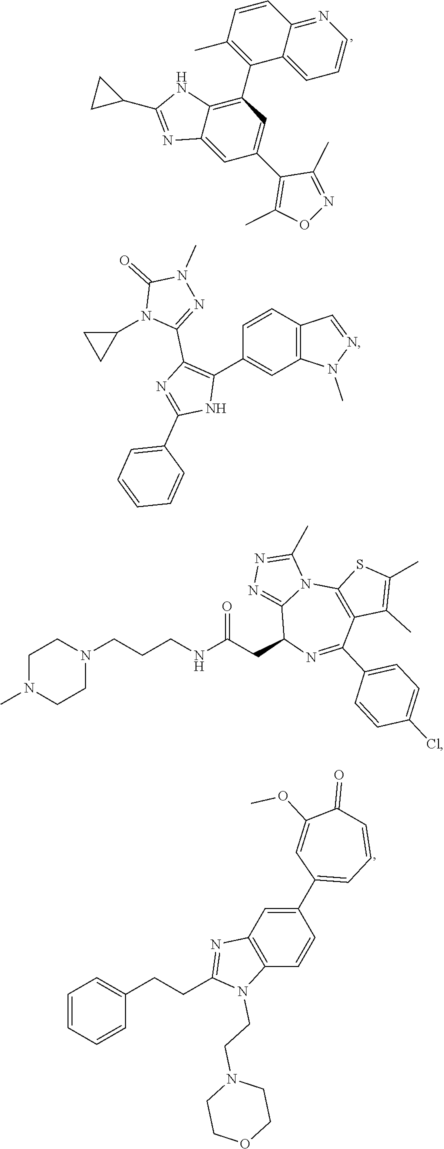

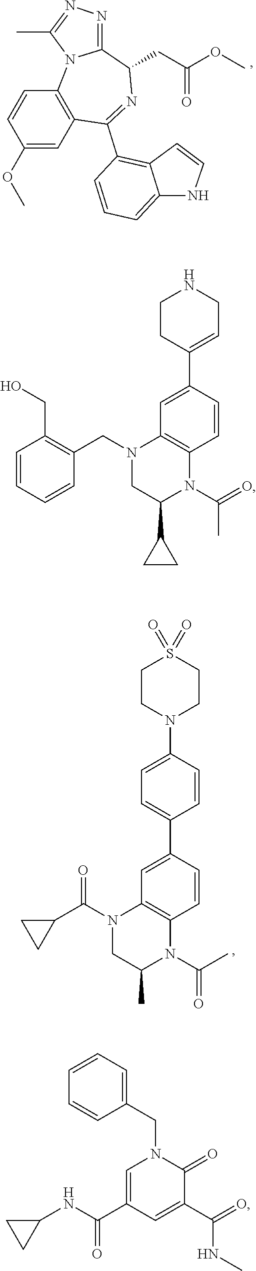 [SMILES: COC(=O)C[C@@H]1N=C(c2cccc3[nH]ccc23)c2cc(OC)ccc2-n2c(C)nnc21 ; CC(=O)N1c2ccc(C3=CCNCC3)cc2N(Cc2ccccc2CO)C[C@@H]1C1CC1 ; CC(=O)N1c2ccc(-c3ccc(N4CCS(=O)(=O)CC4)cc3)cc2N(C(=O)C2CC2)C[C@@H]1C ; CNC(=O)c1cc(C(=O)NC2CC2)cn(Cc2ccccc2)c1=O]

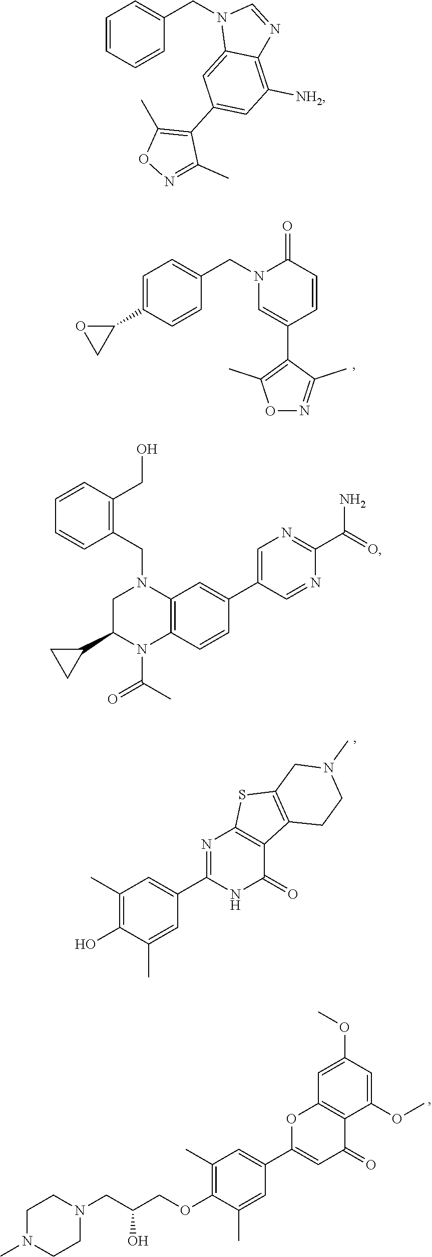

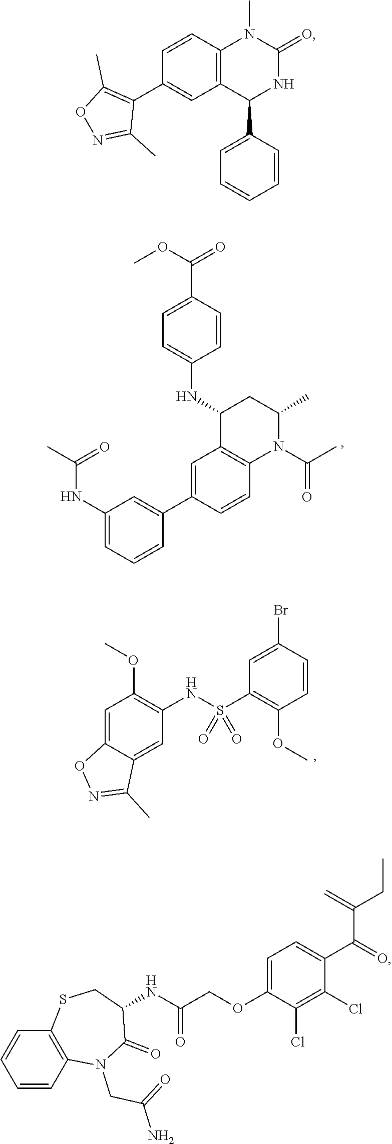 [SMILES: Cc1noc(C)c1-c1ccc2c(c1)[C@H](c1ccccc1)NC(=O)N2C ; COC(=O)c1ccc(N[C@@H]2C[C@H](C)N(C(C)=O)c3ccc(-c4cccc(NC(C)=O)c4)cc32)cc1 ; COc1cc2onc(C)c2cc1NS(=O)(=O)c1cc(Br)ccc1OC ; C=C(CC)C(=O)c1ccc(OCC(=O)N[C@H]2CSc3ccccc3N(CC(N)=O)C2=O)c(Cl)c1Cl]

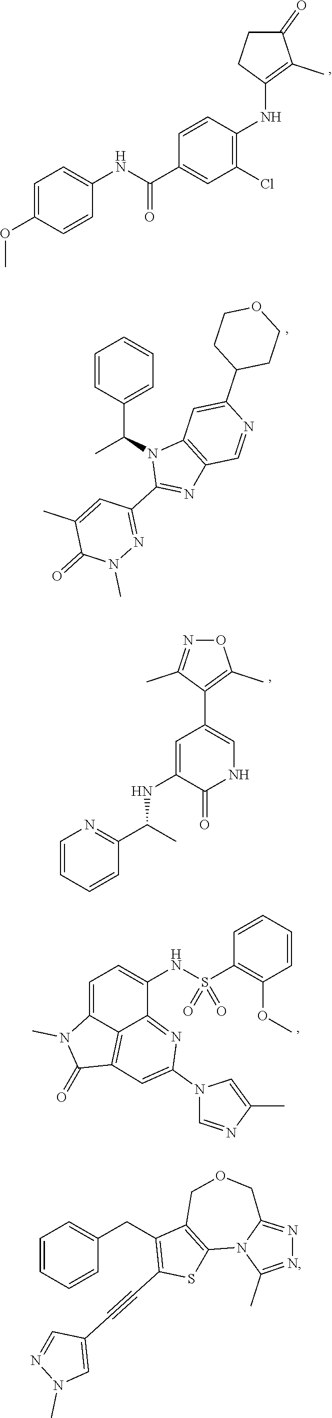

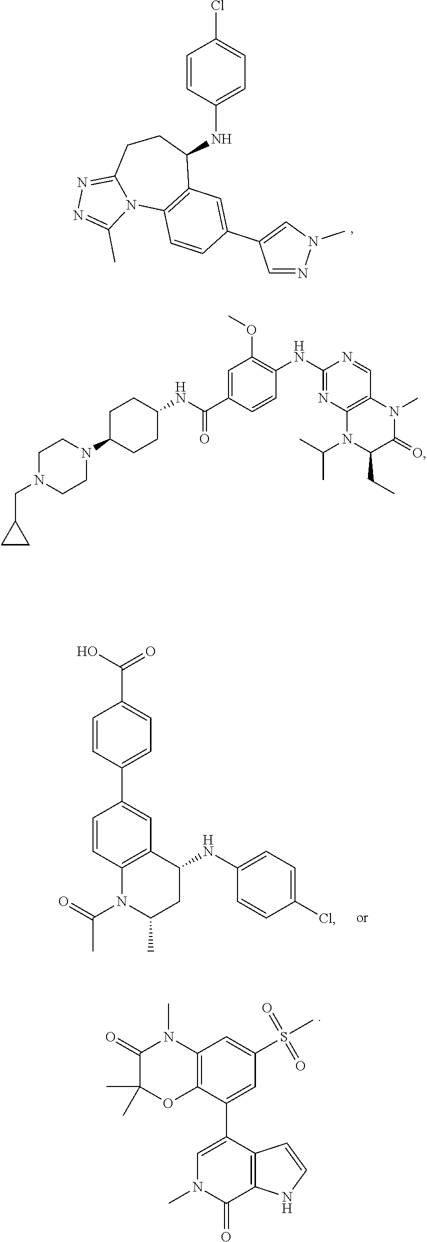

In some embodiments, the recruiting ligand is:

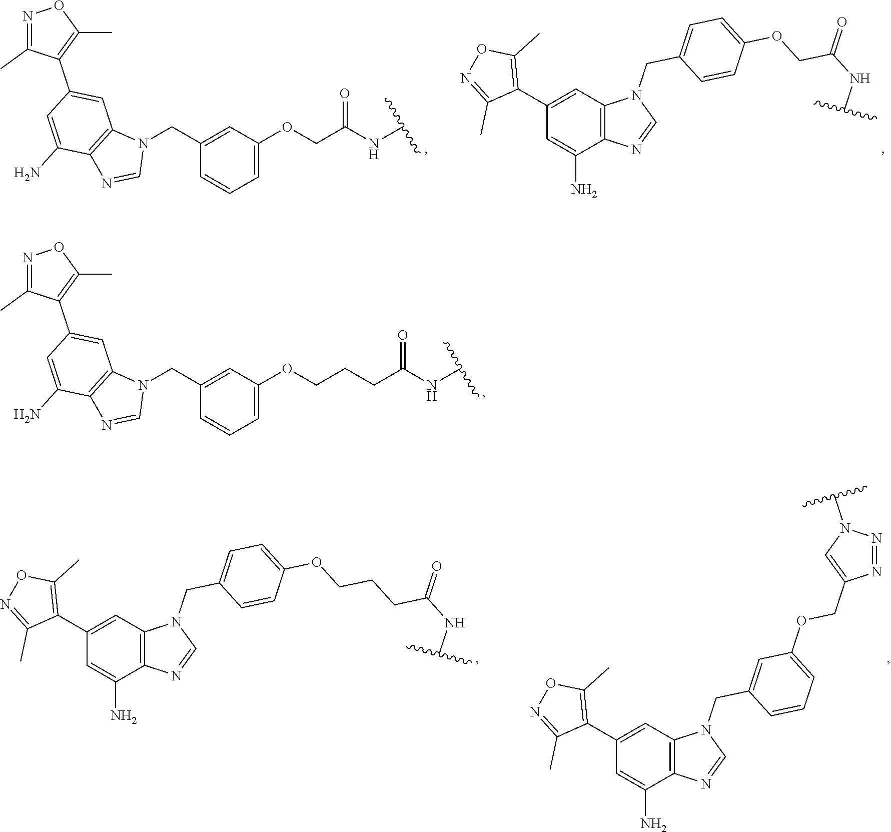 [SMILES: *NC(=O)COc1cccc(Cn2cnc3c(N)cc(-c4c(C)noc4C)cc32)c1 ; *NC(=O)COc1ccc(Cn2cnc3c(N)cc(-c4c(C)noc4C)cc32)cc1 ; *NC(=O)CCCOc1cccc(Cn2cnc3c(N)cc(-c4c(C)noc4C)cc32)c1 ; *NC(=O)CCCOc1ccc(Cn2cnc3c(N)cc(-c4c(C)noc4C)cc32)cc1 ; *n1cc(COc2cccc(Cn3cnc4c(N)cc(-c5c(C)noc5C)cc43)c2)nn1]

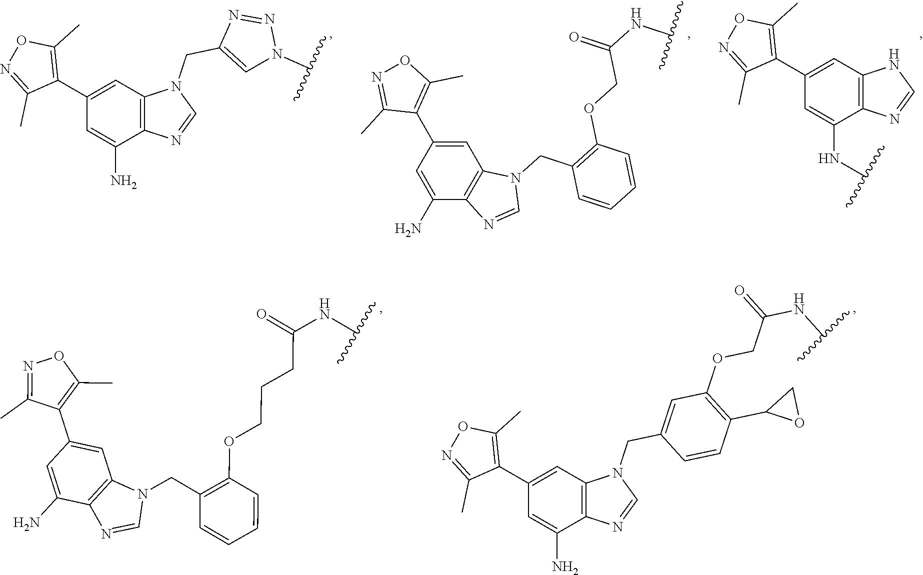 [SMILES: *n1cc(Cn2cnc3c(N)cc(-c4c(C)noc4C)cc32)nn1 ; *NC(=O)COc1ccccc1Cn1cnc2c(N)cc(-c3c(C)noc3C)cc21 ; *Nc1cc(-c2c(C)noc2C)cc2[nH]cnc12 ; *NC(=O)CCCOc1ccccc1Cn1cnc2c(N)cc(-c3c(C)noc3C)cc21 ; *NC(=O)COc1cc(Cn2cnc3c(N)cc(-c4c(C)noc4C)cc32)ccc1C1CO1]

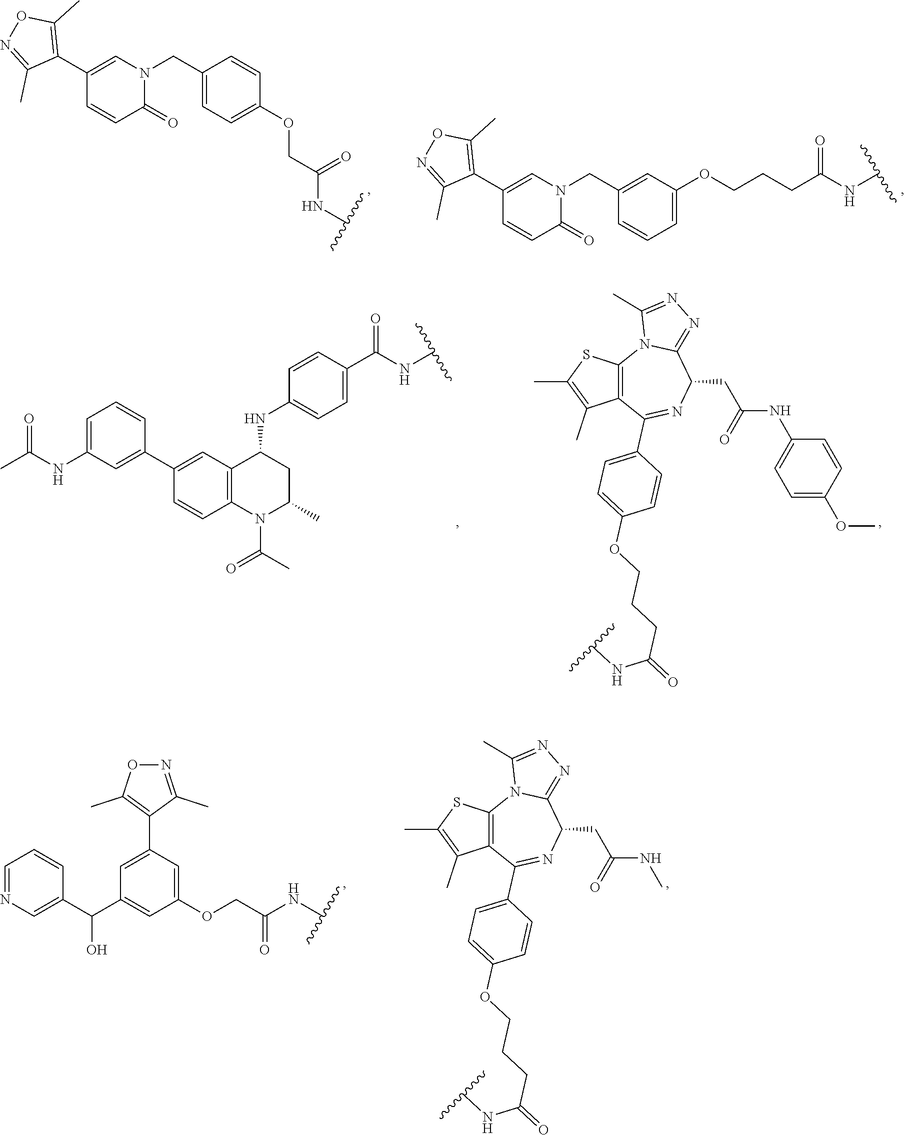

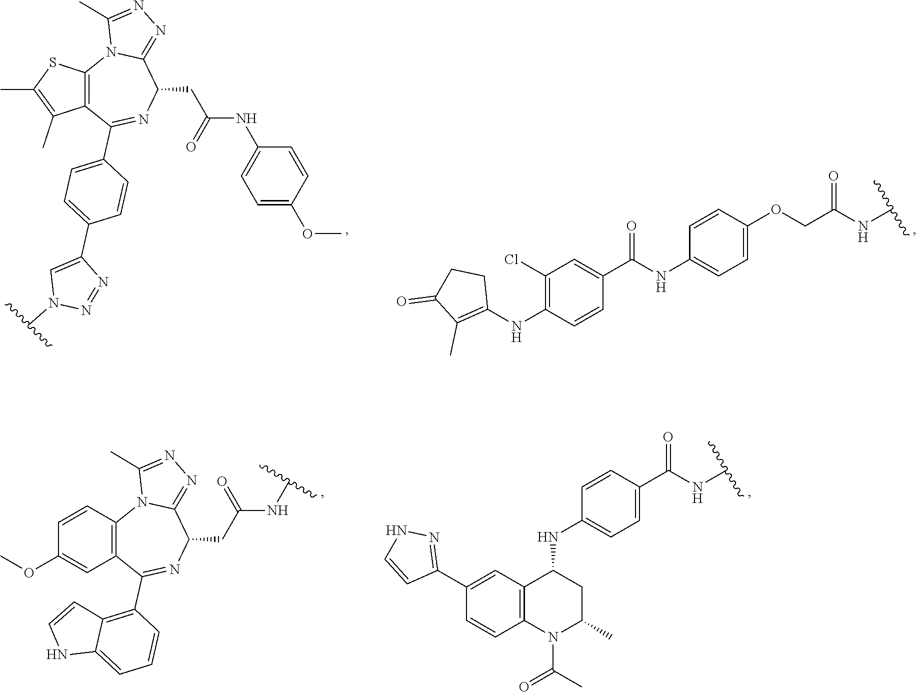

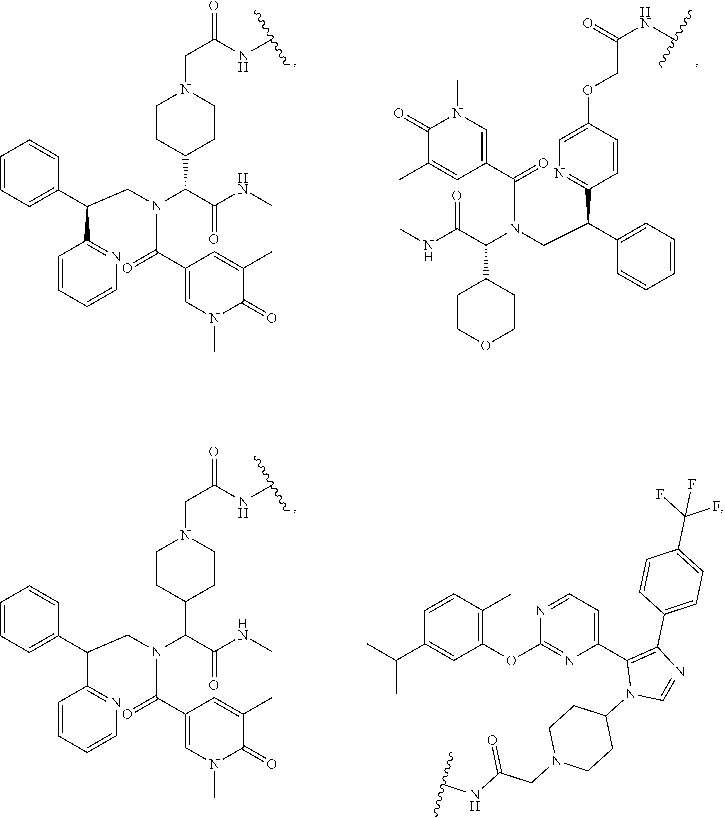 [SMILES: *NC(=O)CN1CCC([C@H](C(=O)NC)N(C[C@H](c2ccccc2)c2ccccn2)C(=O)c2cc(C)c(=O)n(C)c2)CC1 ; *NC(=O)COc1ccc([C@H](CN(C(=O)c2cc(C)c(=O)n(C)c2)[C@@H](C(=O)NC)C2CCOCC2)c2ccccc2)nc1 ; *NC(=O)CN1CCC(C(C(=O)NC)N(CC(c2ccccc2)c2ccccn2)C(=O)c2cc(C)c(=O)n(C)c2)CC1 ; *NC(=O)CN1CCC(n2cnc(-c3ccc(C(F)(F)F)cc3)c2-c2ccnc(Oc3cc(C(C)C)ccc3C)n2)CC1]

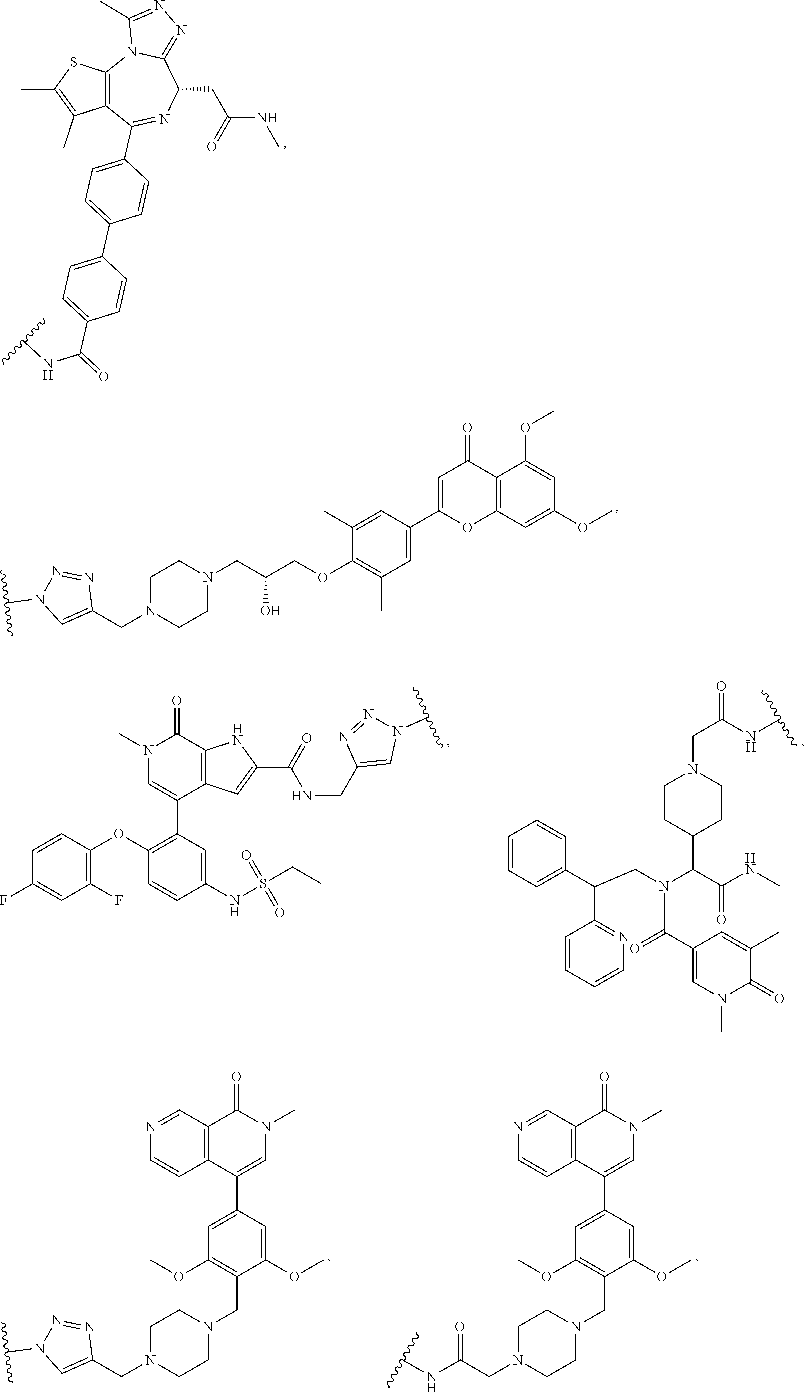

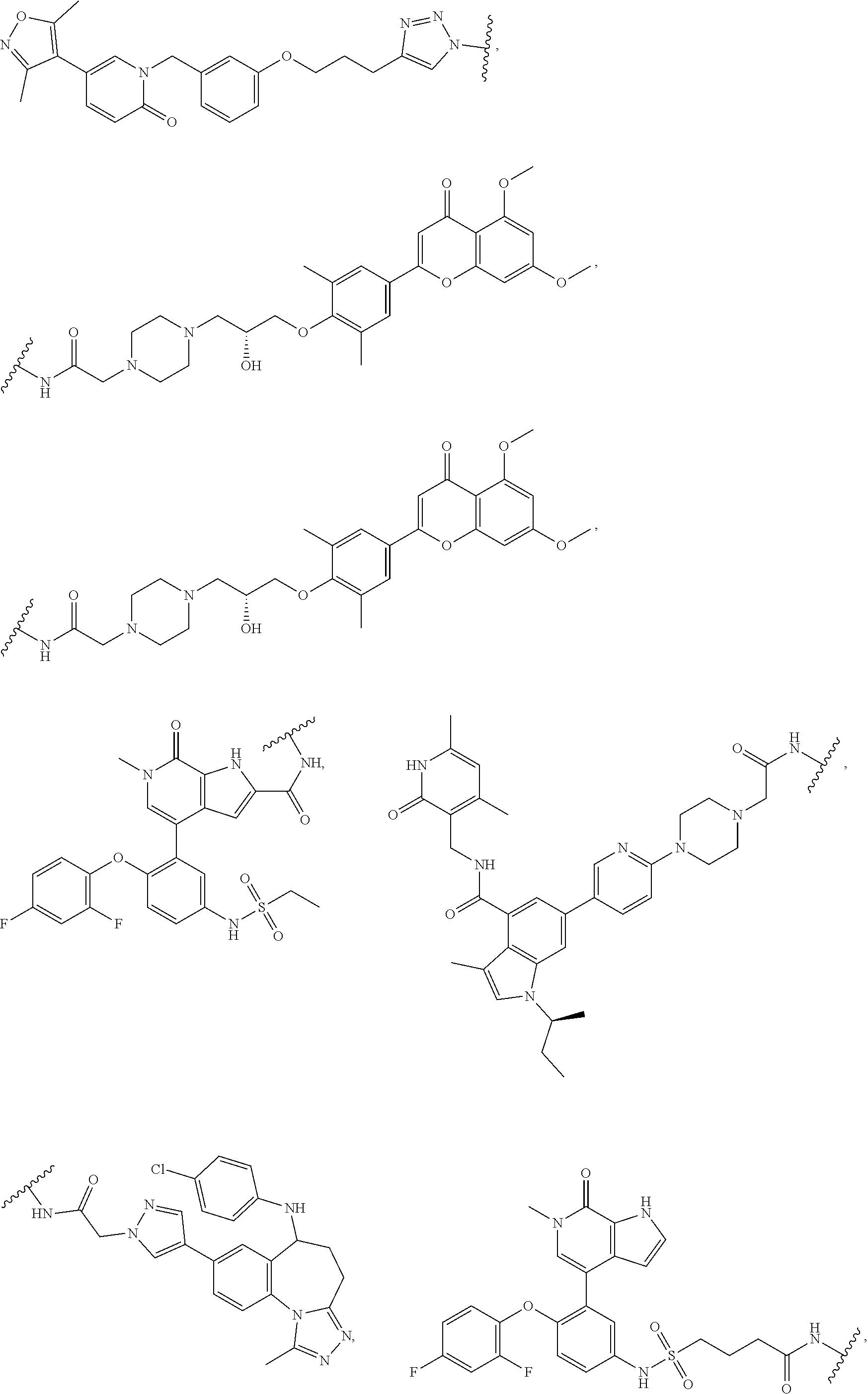

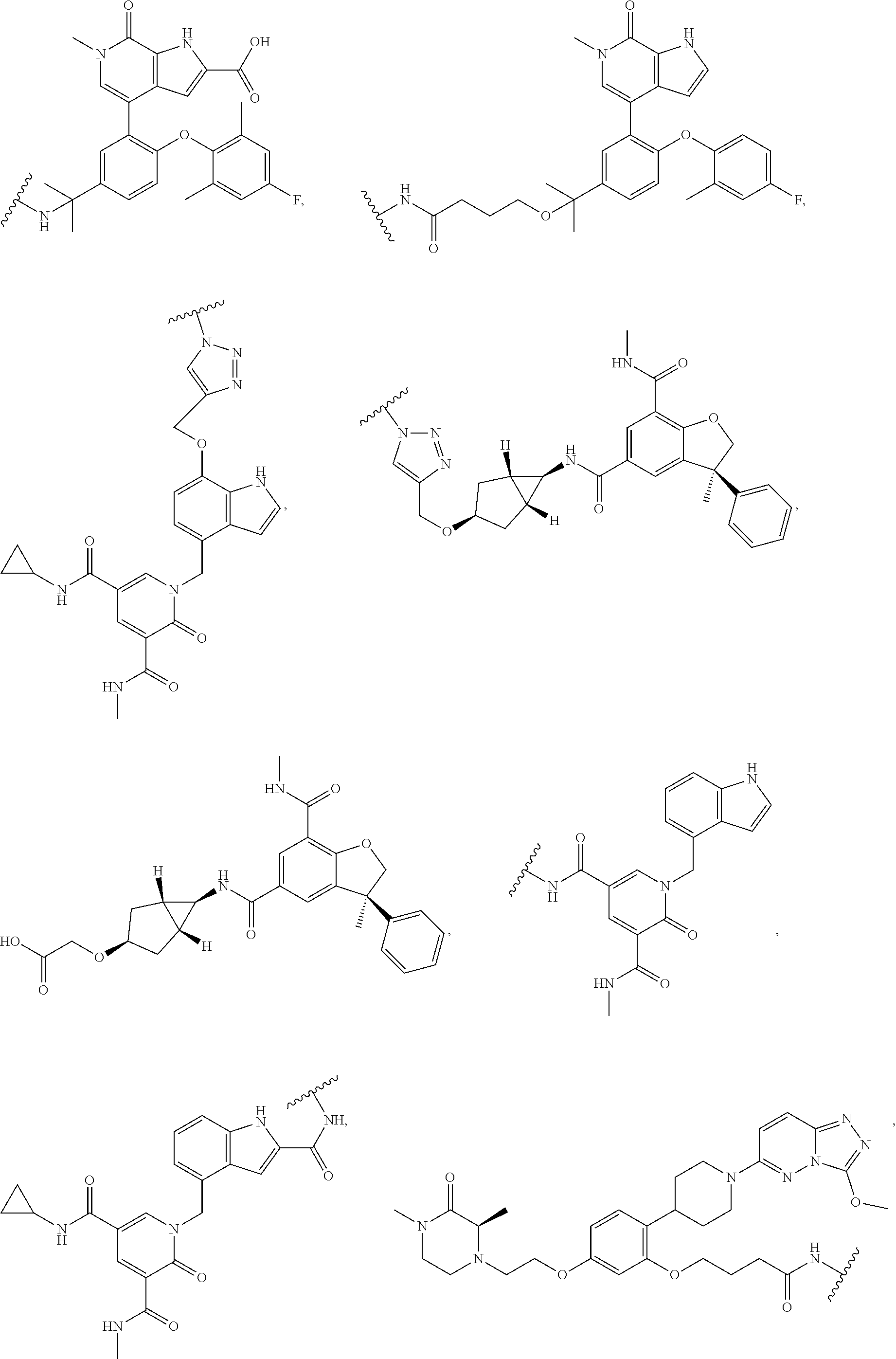

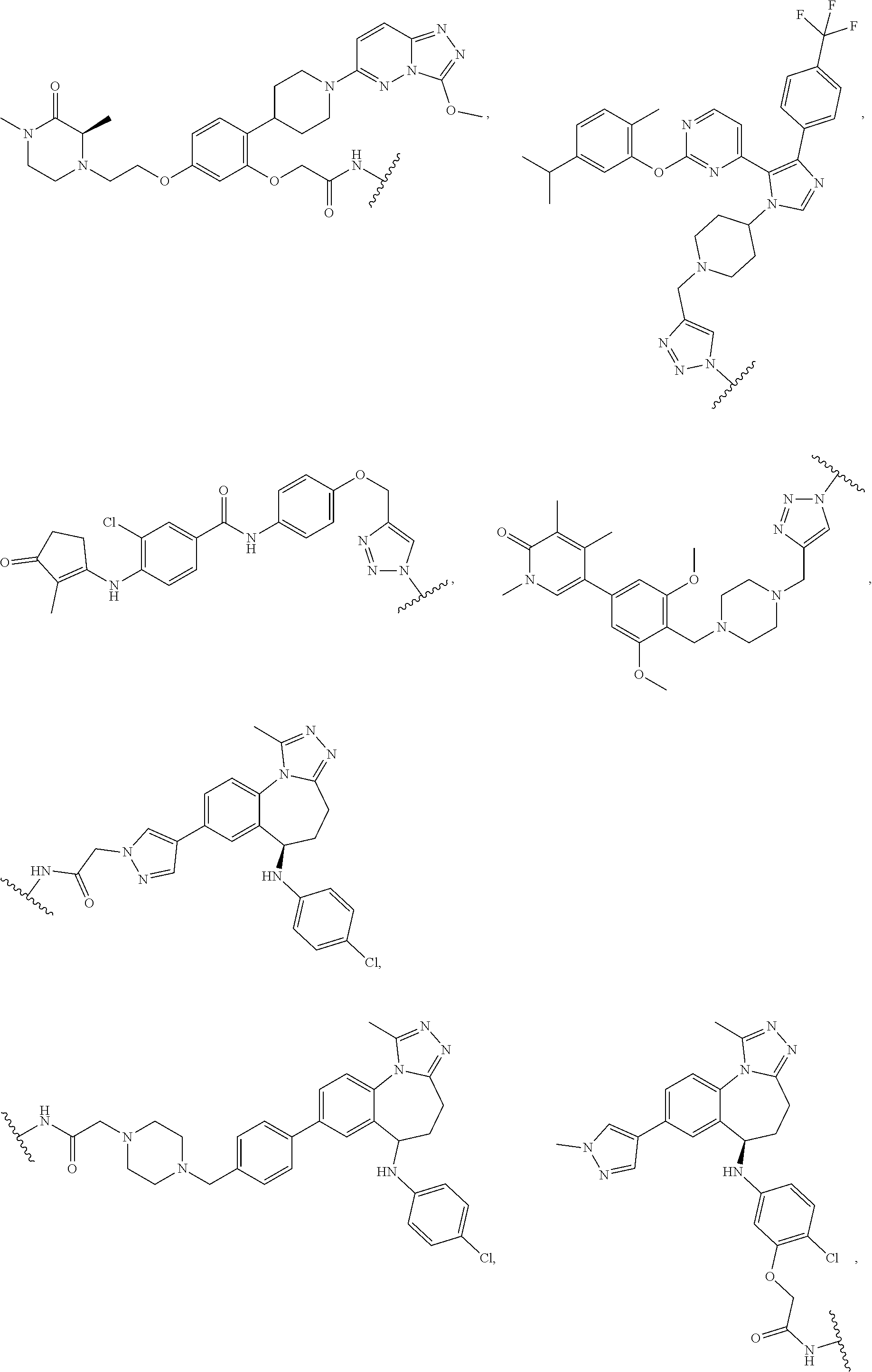

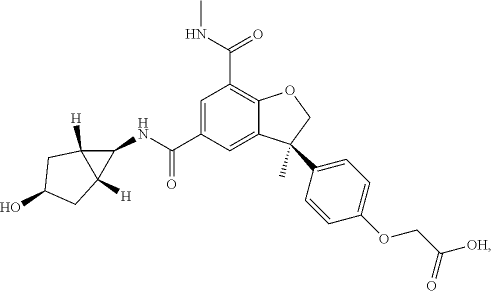 [SMILES: CNC(=O)c1cc(C(=O)N[C@H]2[C@@H]3C[C@@H](O)C[C@@H]32)cc2c1OC[C@@]2(C)c1ccc(OCC(=O)O)cc1]

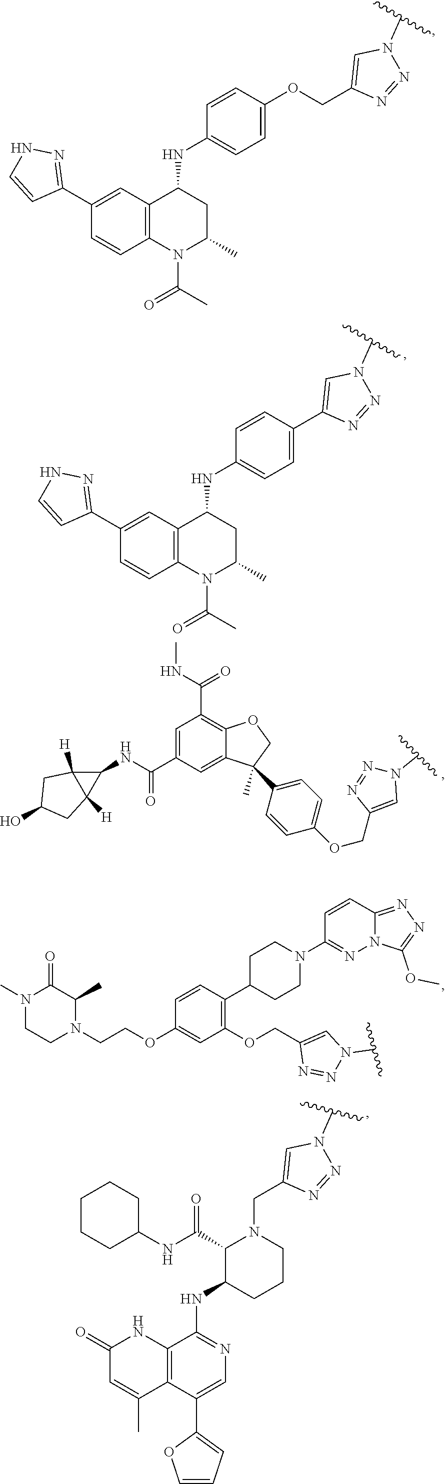

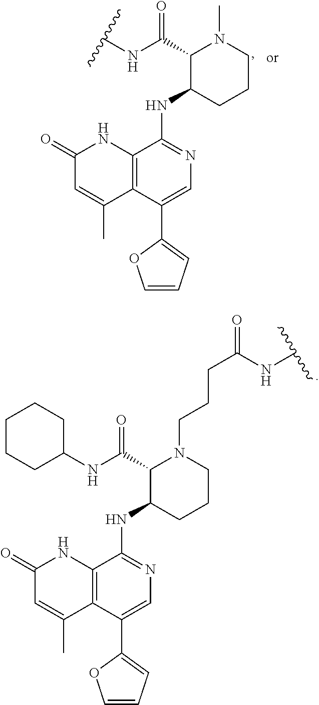 [SMILES: *NC(=O)[C@H]1[C@H](Nc2ncc(-c3ccco3)c3c(C)cc(=O)[nH]c23)CCCN1C ; *NC(=O)CCCN1CCC[C@@H](Nc2ncc(-c3ccco3)c3c(C)cc(=O)[nH]c23)[C@@H]1C(=O)NC1CCCCC1]

In some embodiments, the recruiting ligand is:

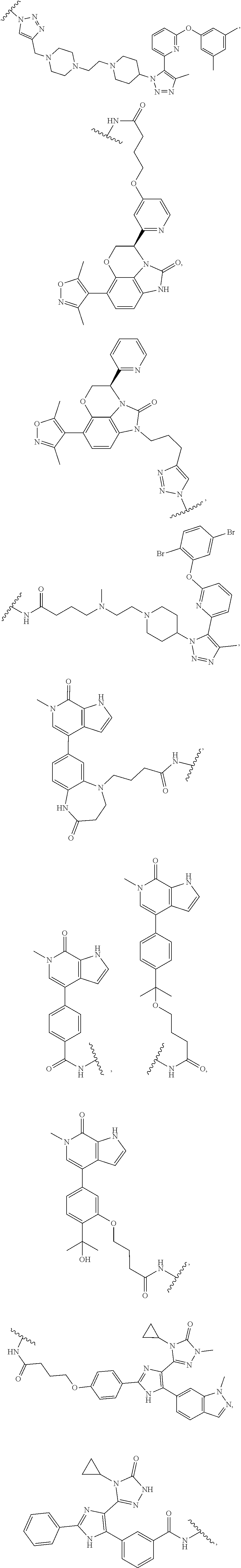

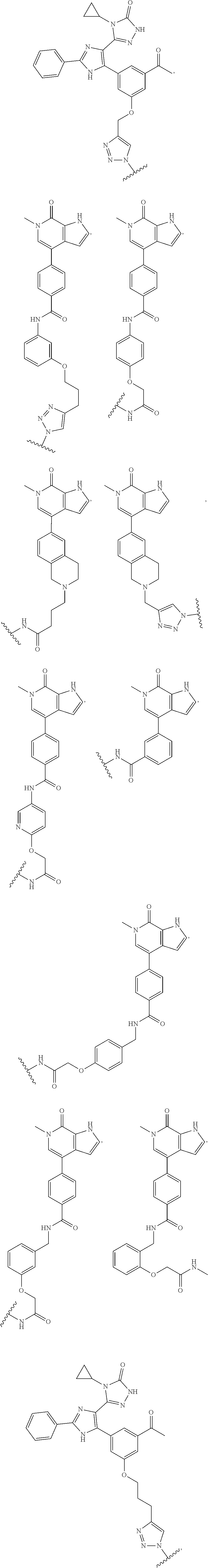

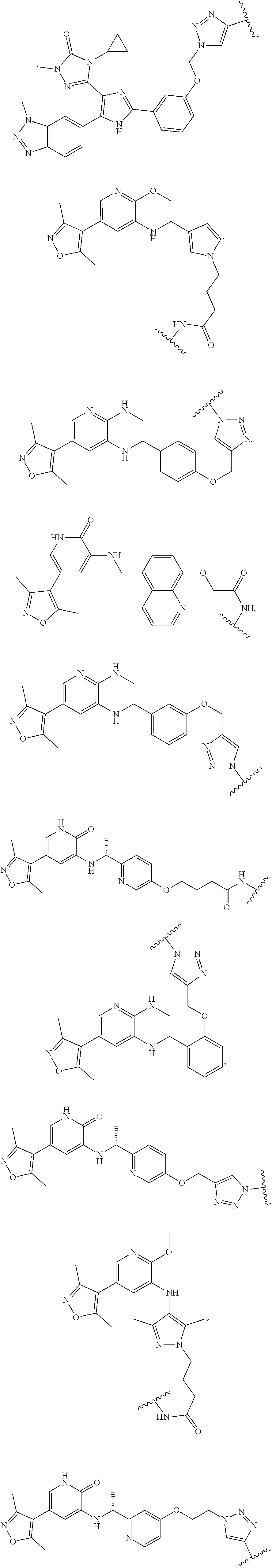

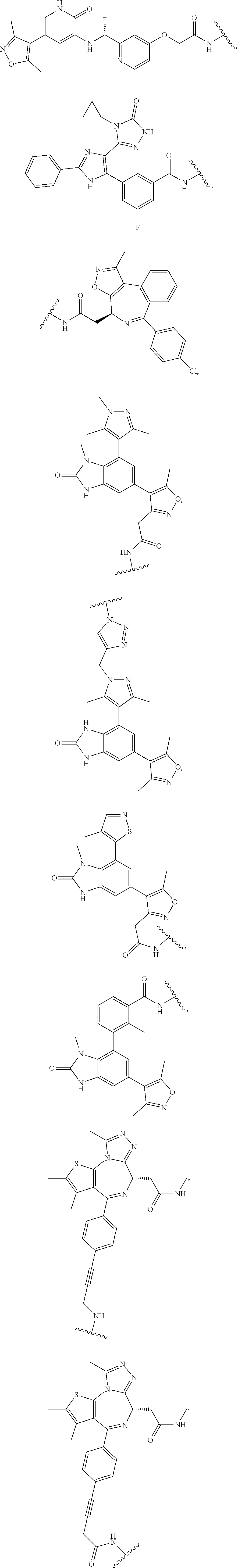

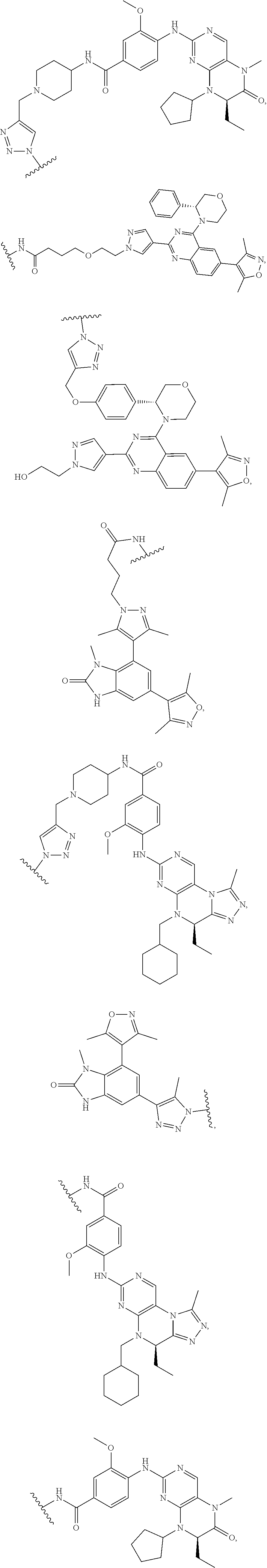

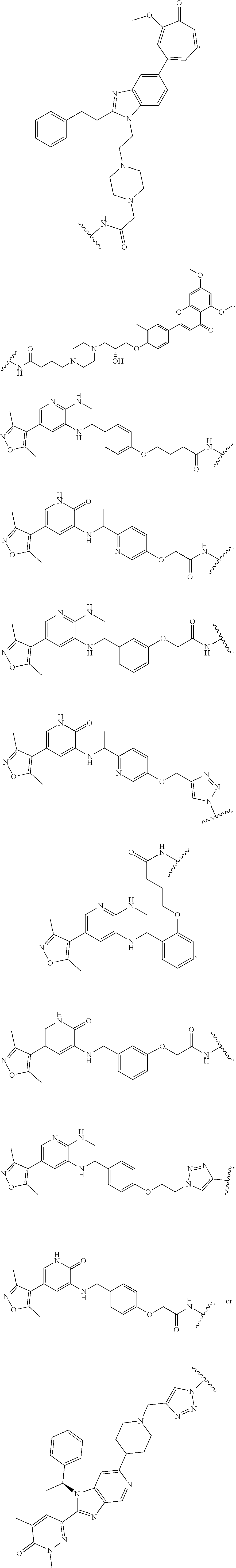

In some embodiments, the recruiting ligand is:

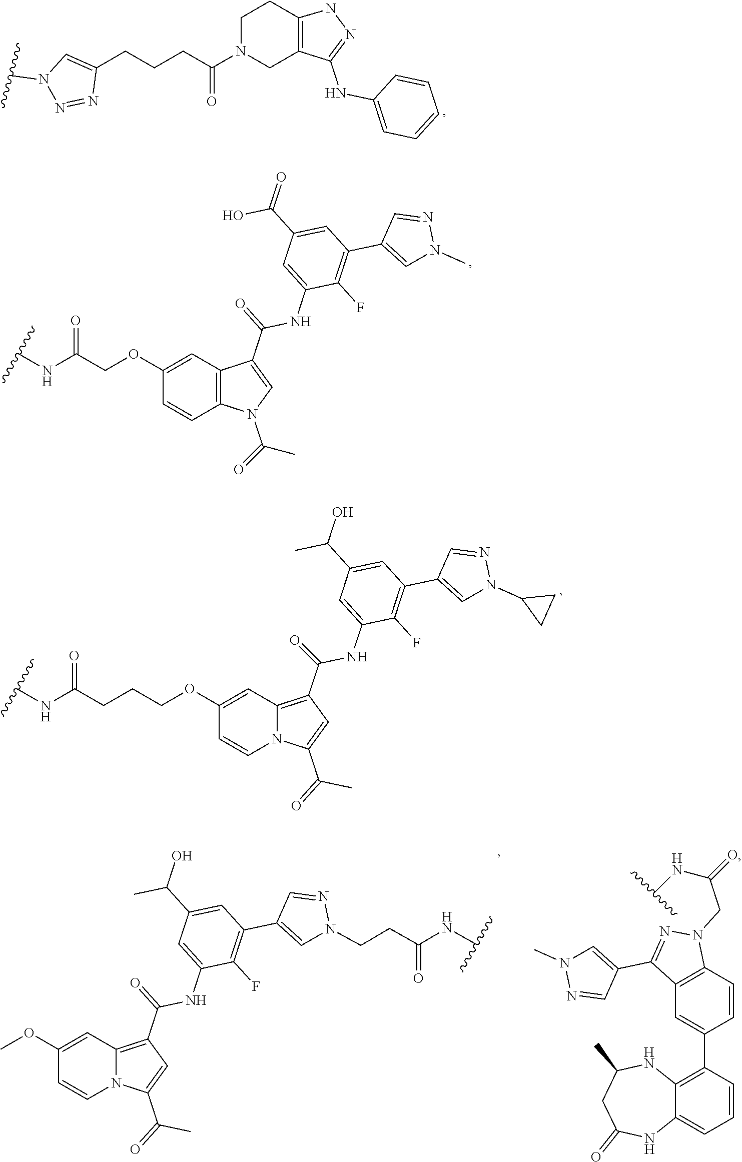

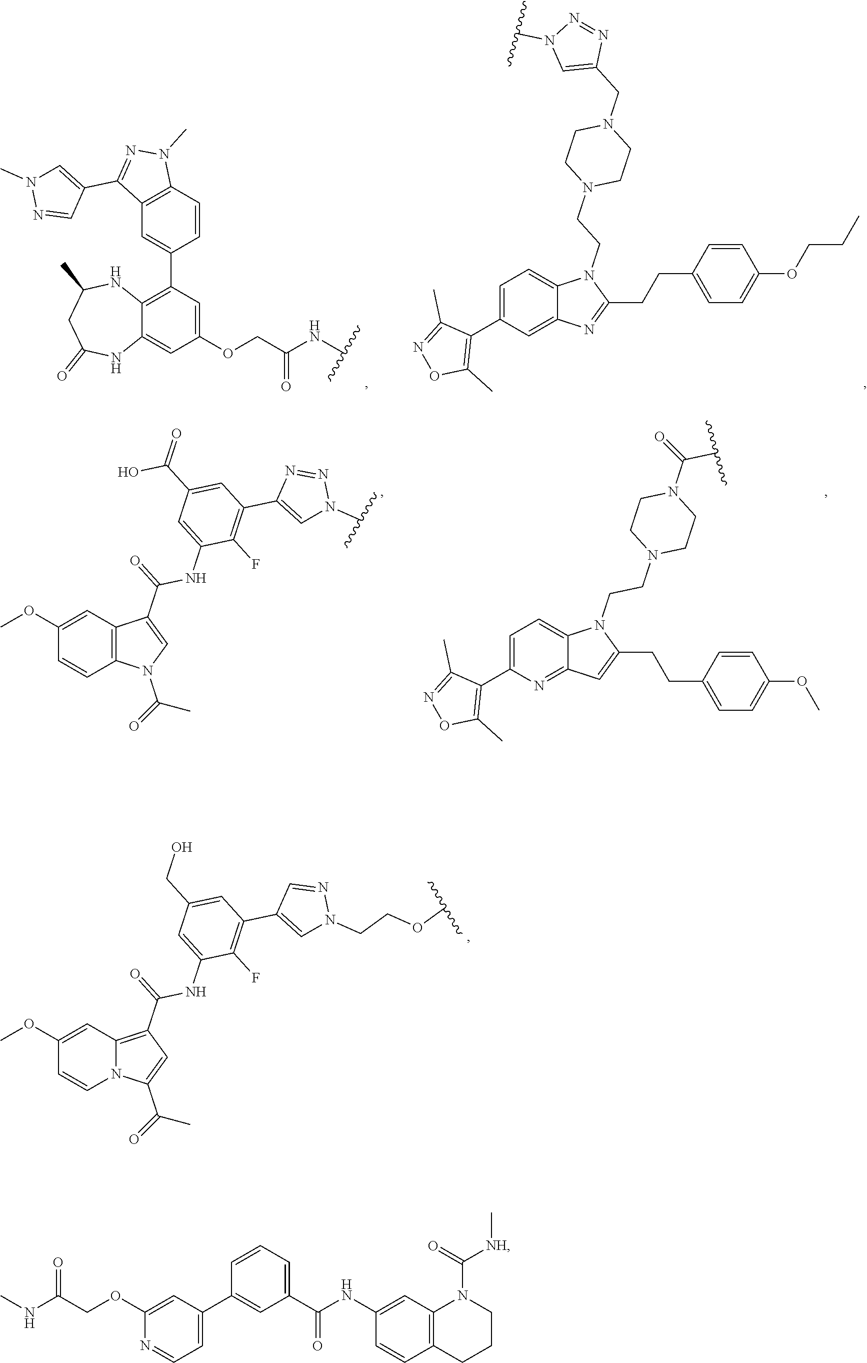 [SMILES: *NC(=O)COc1cc2c(c(-c3ccc4c(c3)c(-c3cnn(C)c3)nn4C)c1)N[C@H](C)CC(=O)N2 ; *n1cc(CN2CCN(CCn3c(CCc4ccc(OCCC)cc4)nc4cc(-c5c(C)noc5C)ccc43)CC2)nn1 ; *n1cc(-c2cc(C(=O)O)cc(NC(=O)c3cn(C(C)=O)c4ccc(OC)cc34)c2F)nn1 ; *C(=O)N1CCN(CCn2c(CCc3ccc(OC)cc3)cc3nc(-c4c(C)noc4C)ccc32)CC1 ; *OCCn1cc(-c2cc(CO)cc(NC(=O)c3cc(C(C)=O)n4ccc(OC)cc34)c2F)cn1 ; CNC(=O)COc1cc(-c2cccc(C(=O)Nc3ccc4c(c3)N(C(=O)NC)CCC4)c2)ccn1]

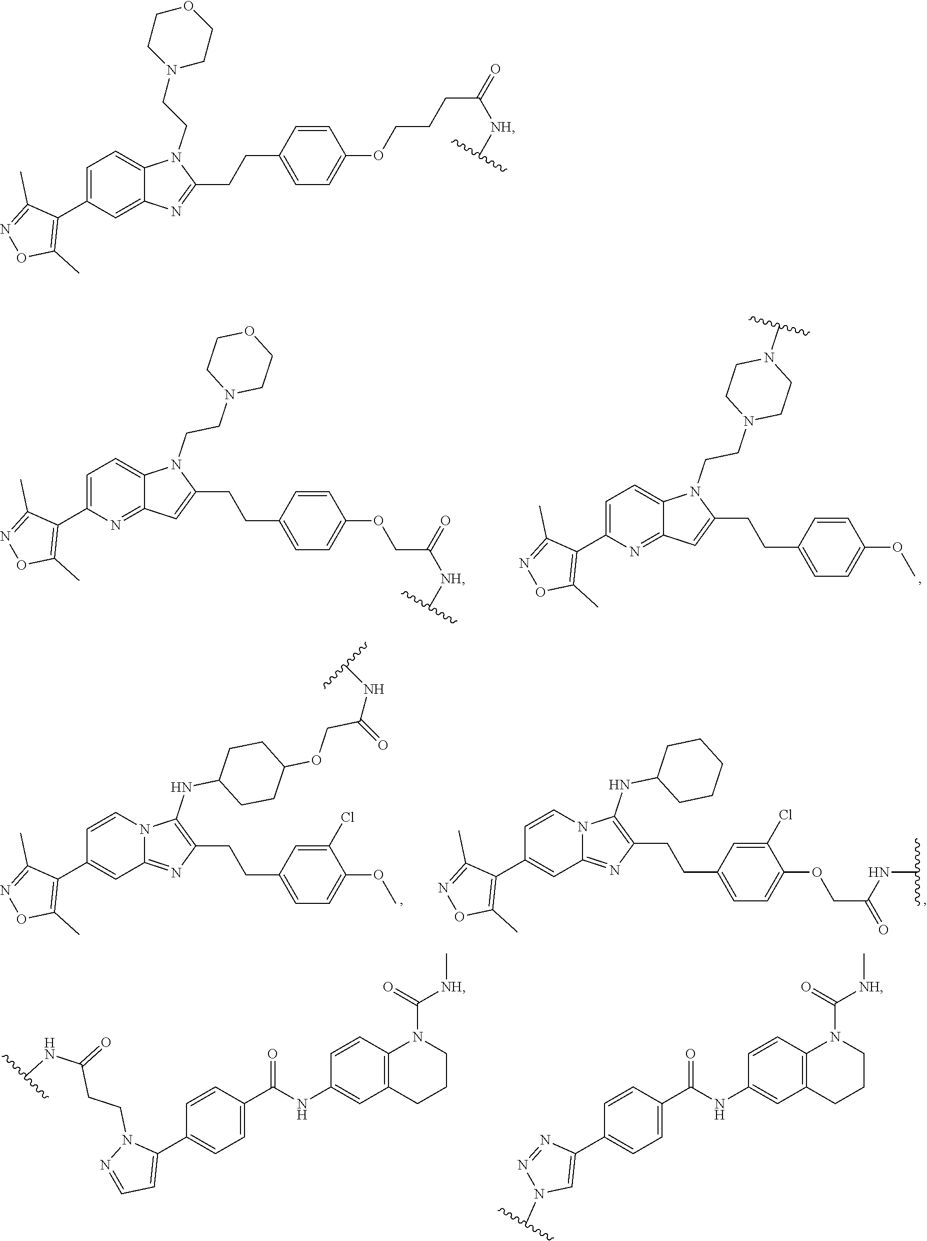

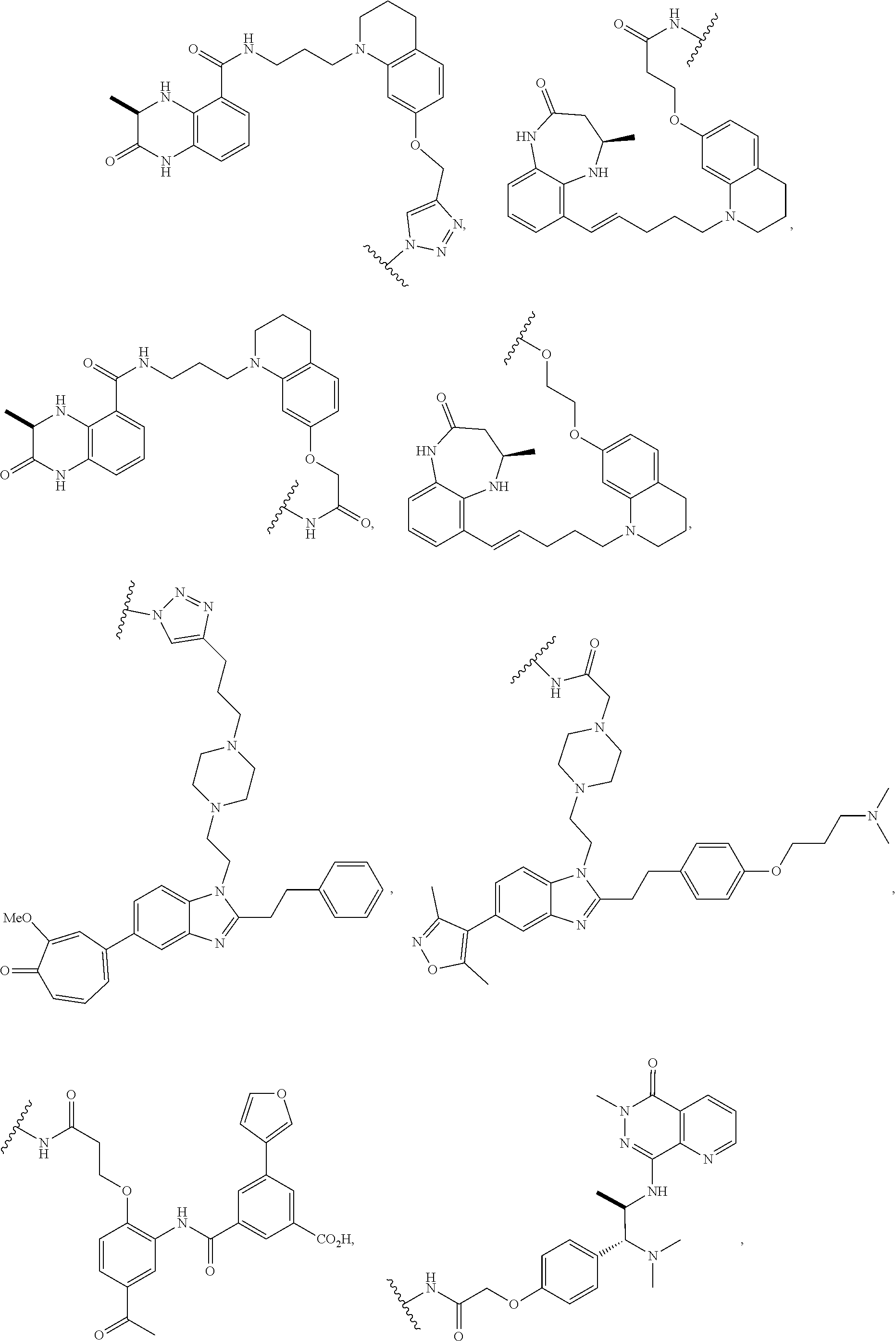

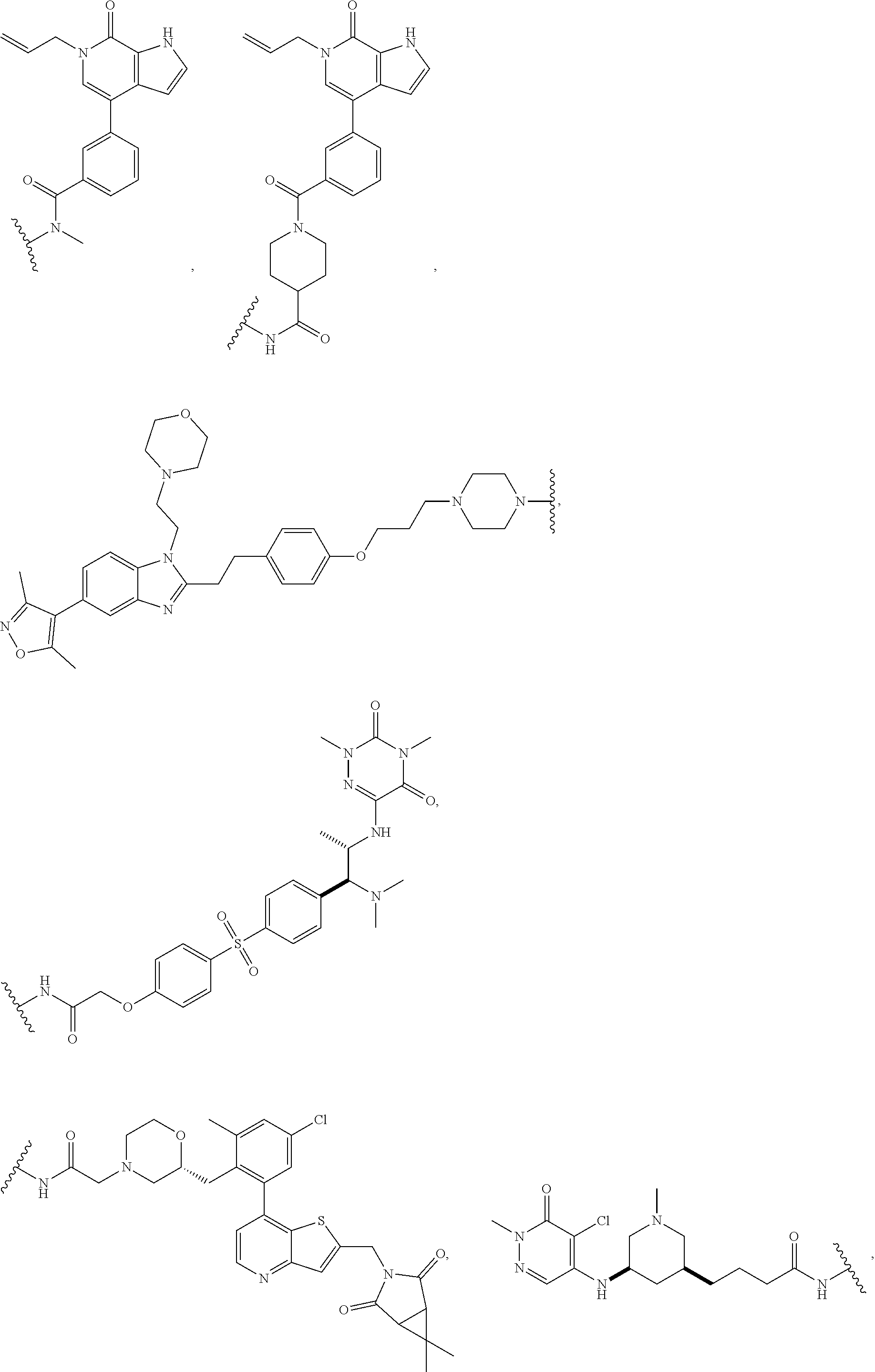 [SMILES: *N(C)C(=O)c1cccc(-c2cn(CC=C)c(=O)c3[nH]ccc23)c1 ; *NC(=O)C1CCN(C(=O)c2cccc(-c3cn(CC=C)c(=O)c4[nH]ccc34)c2)CC1 ; *N1CCN(CCCOc2ccc(CCc3nc4cc(-c5c(C)noc5C)ccc4n3CCN3CCOCC3)cc2)CC1 ; *NC(=O)COc1ccc(S(=O)(=O)c2ccc([C@@H]([C@H](C)Nc3nn(C)c(=O)n(C)c3=O)N(C)C)cc2)cc1 ; *NC(=O)CN1CCO[C@H](Cc2c(C)cc(Cl)cc2-c2ccnc3cc(CN4C(=O)C5C(C4=O)C5(C)C)sc23)C1 ; *NC(=O)CCC[C@H]1C[C@@H](Nc2cnn(C)c(=O)c2Cl)CN(C)C1]

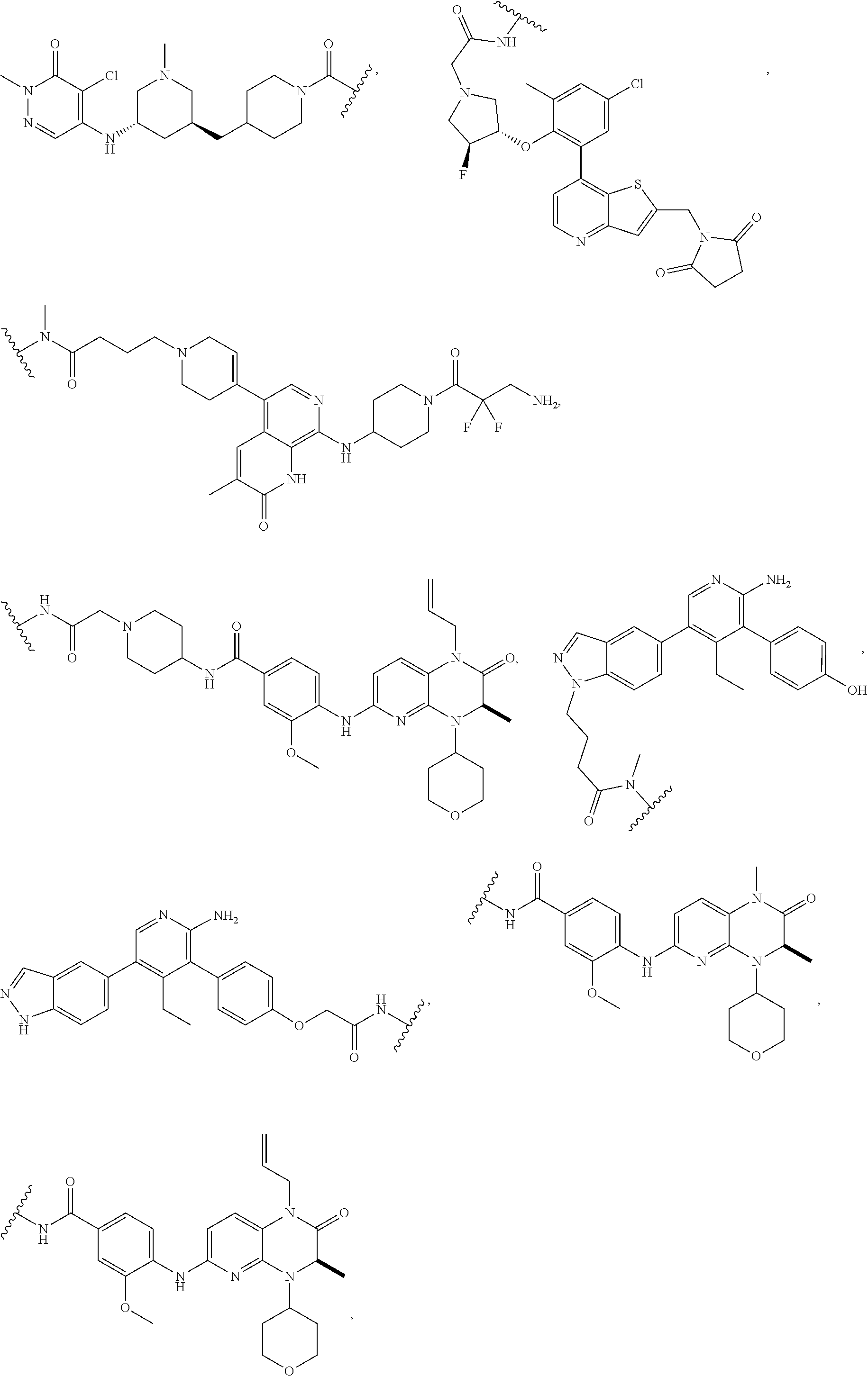

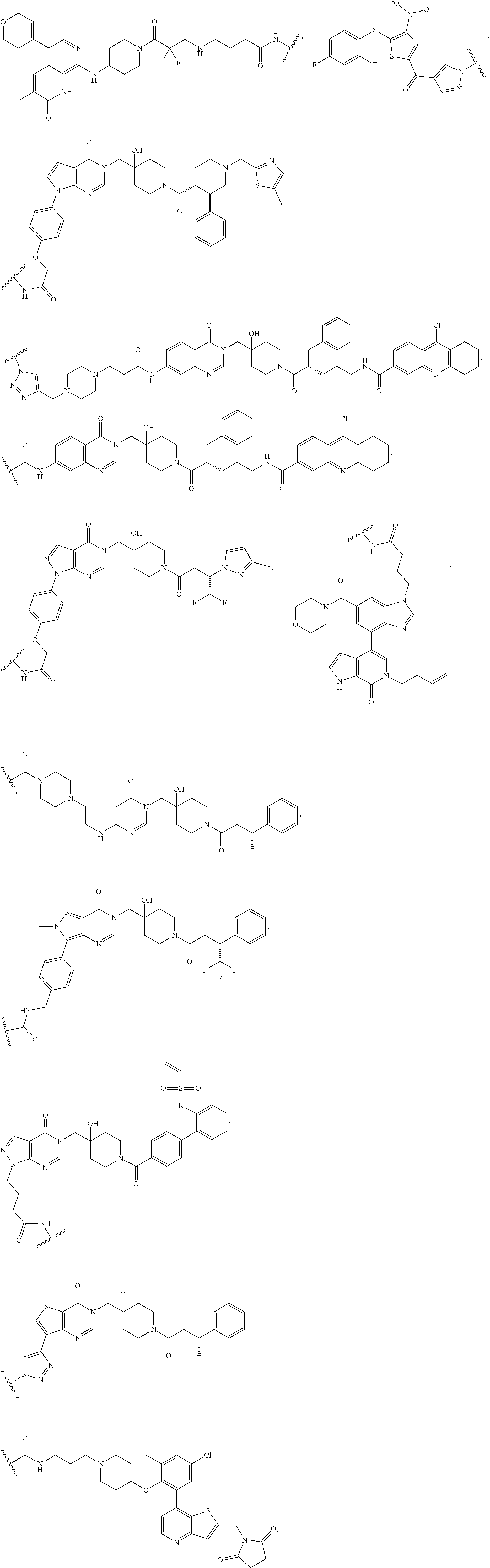

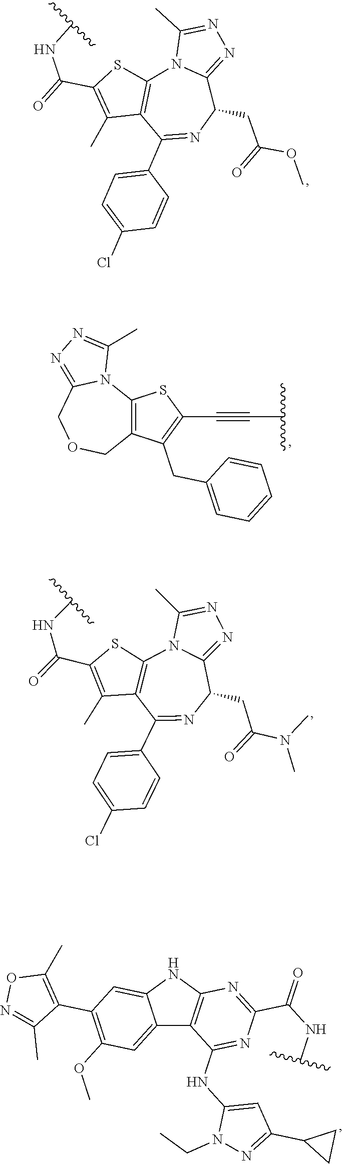 [SMILES: *NC(=O)c1sc2c(c1C)C(c1ccc(Cl)cc1)=N[C@@H](CC(=O)OC)c1nnc(C)n1-2 ; *C#Cc1sc2c(c1Cc1ccccc1)COCc1nnc(C)n1-2 ; *NC(=O)c1sc2c(c1C)C(c1ccc(Cl)cc1)=N[C@@H](CC(=O)N(C)C)c1nnc(C)n1-2 ; *NC(=O)c1nc(Nc2cc(C3CC3)nn2CC)c2c(n1)[nH]c1cc(-c3c(C)noc3C)c(OC)cc12]

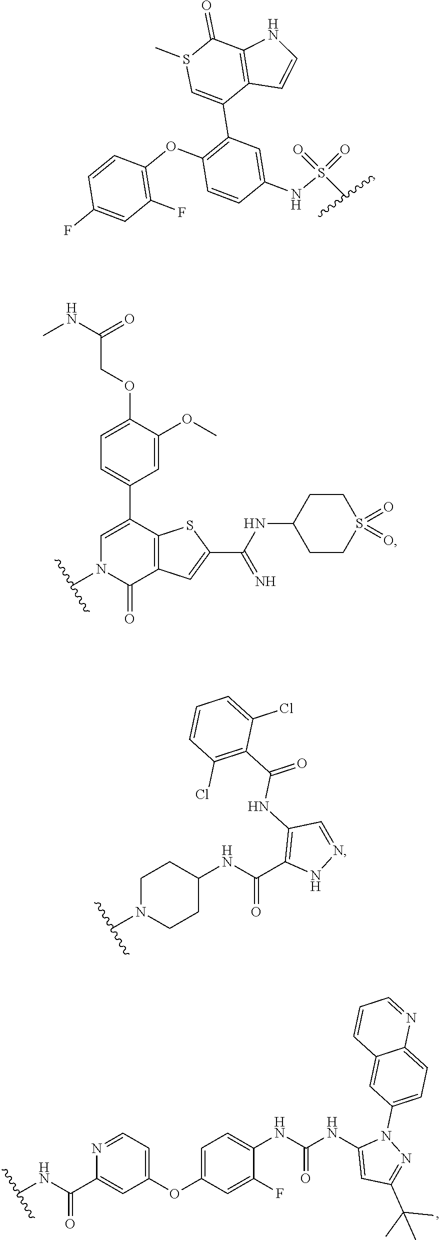

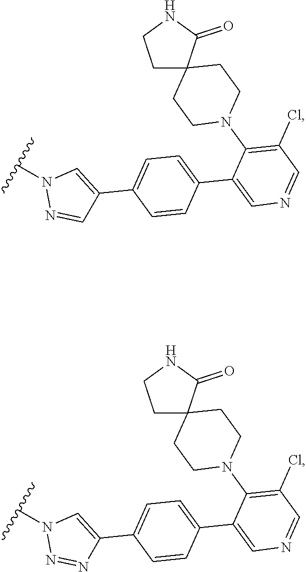 [SMILES: *n1cc(-c2ccc(-c3cncc(Cl)c3N3CCC4(CCNC4=O)CC3)cc2)cn1 ; *n1cc(-c2ccc(-c3cncc(Cl)c3N3CCC4(CCNC4=O)CC3)cc2)nn1]

In some embodiments, the recruiting ligand is:

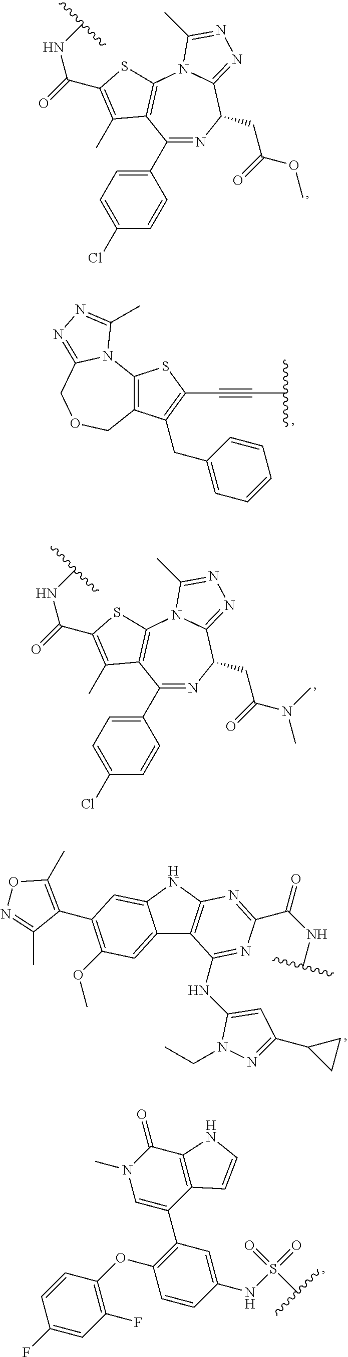 [SMILES: *NC(=O)c1sc2c(c1C)C(c1ccc(Cl)cc1)=N[C@@H](CC(=O)OC)c1nnc(C)n1-2 ; *C#Cc1sc2c(c1Cc1ccccc1)COCc1nnc(C)n1-2 ; *NC(=O)c1sc2c(c1C)C(c1ccc(Cl)cc1)=N[C@@H](CC(=O)N(C)C)c1nnc(C)n1-2 ; *NC(=O)c1nc(Nc2cc(C3CC3)nn2CC)c2c(n1)[nH]c1cc(-c3c(C)noc3C)c(OC)cc12 ; *S(=O)(=O)Nc1ccc(Oc2ccc(F)cc2F)c(-c2cn(C)c(=O)c3[nH]ccc23)c1]

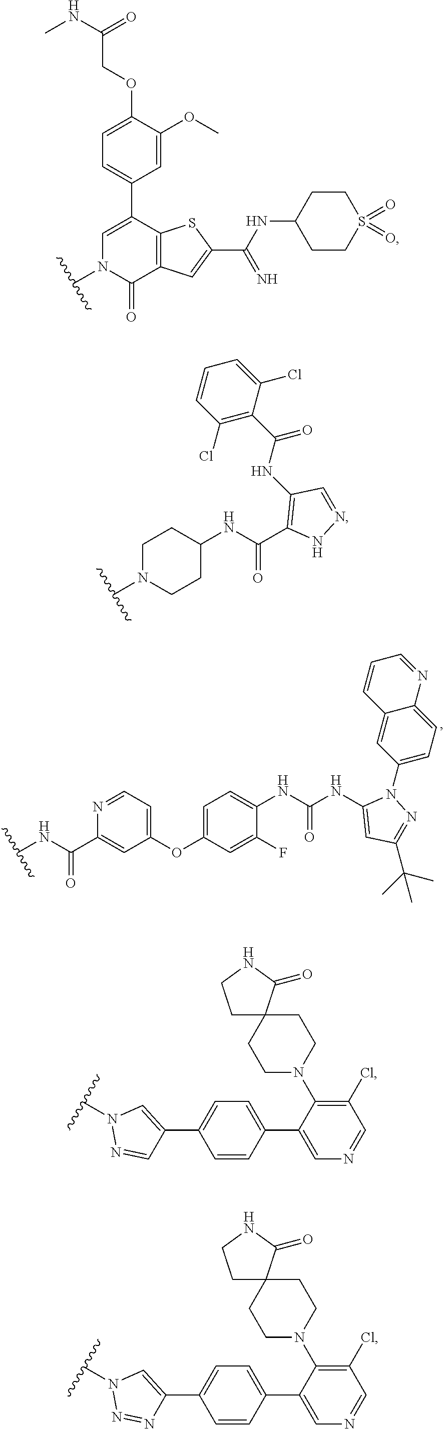 [SMILES: *n1cc(-c2ccc(OCC(=O)NC)c(OC)c2)c2sc(C(=N)NC3CCS(=O)(=O)CC3)cc2c1=O ; *N1CCC(NC(=O)c2[nH]ncc2NC(=O)c2c(Cl)cccc2Cl)CC1 ; *NC(=O)c1cc(Oc2ccc(NC(=O)Nc3cc(C(C)(C)C)nn3-c3ccc4ncccc4c3)c(F)c2)ccn1 ; *n1cc(-c2ccc(-c3cncc(Cl)c3N3CCC4(CCNC4=O)CC3)cc2)cn1 ; *n1cc(-c2ccc(-c3cncc(Cl)c3N3CCC4(CCNC4=O)CC3)cc2)nn1]

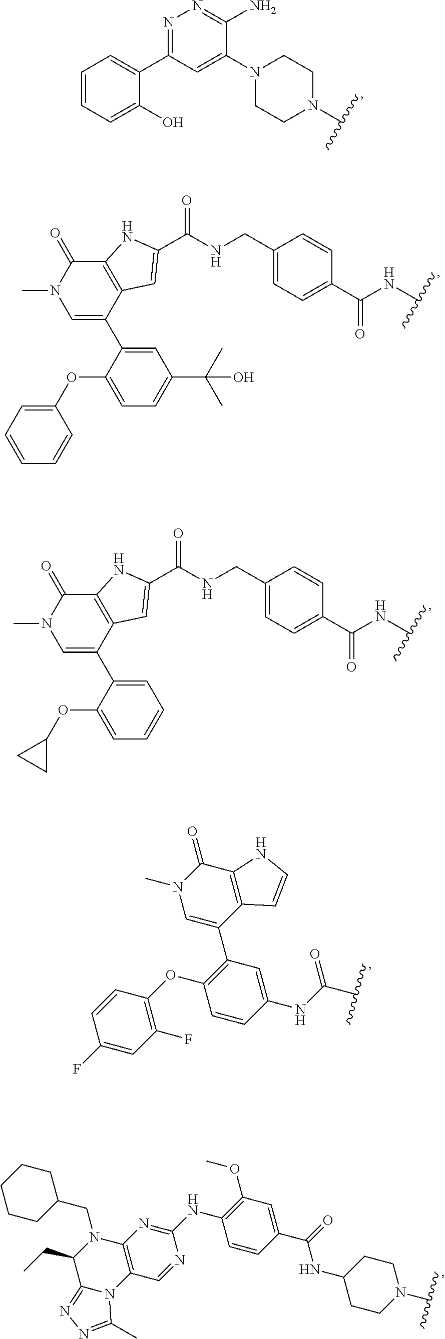 [SMILES: *N1CCN(c2cc(-c3ccccc3O)nnc2N)CC1 ; *NC(=O)c1ccc(CNC(=O)c2cc3c(-c4cc(C(C)(C)O)ccc4Oc4ccccc4)cn(C)c(=O)c3[nH]2)cc1 ; *NC(=O)c1ccc(CNC(=O)c2cc3c(-c4ccccc4OC4CC4)cn(C)c(=O)c3[nH]2)cc1 ; *C(=O)Nc1ccc(Oc2ccc(F)cc2F)c(-c2cn(C)c(=O)c3[nH]ccc23)c1 ; *N1CCC(NC(=O)c2ccc(Nc3ncc4c(n3)N(CC3CCCCC3)[C@H](CC)c3nnc(C)n3-4)c(OC)c2)CC1]

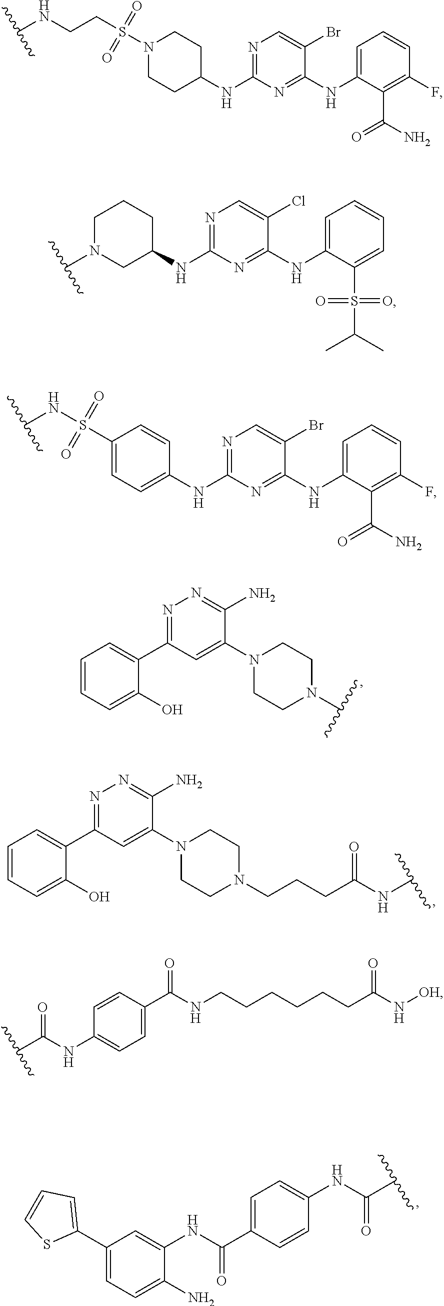

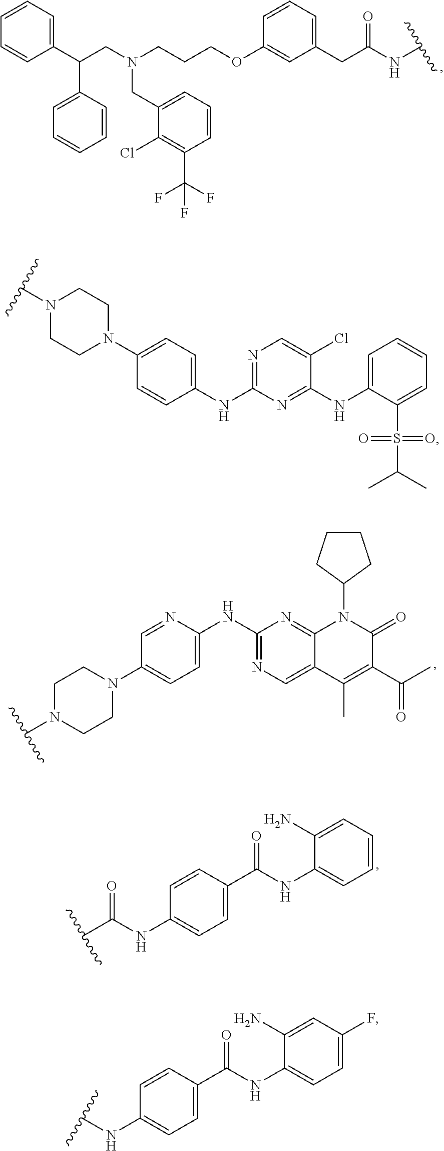

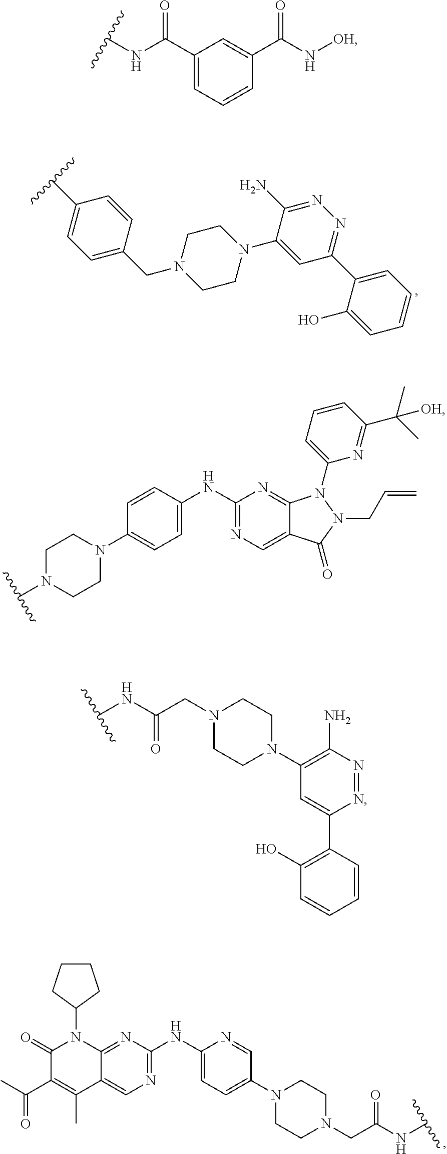 [SMILES: *NC(=O)c1cccc(C(=O)NO)c1 ; *c1ccc(CN2CCN(c3cc(-c4ccccc4O)nnc3N)CC2)cc1 ; *N1CCN(c2ccc(Nc3ncc4c(=O)n(CC=C)n(-c5cccc(C(C)(C)O)n5)c4n3)cc2)CC1 ; *NC(=O)CN1CCN(c2cc(-c3ccccc3O)nnc2N)CC1 ; *NC(=O)CN1CCN(c2ccc(Nc3ncc4c(C)c(C(C)=O)c(=O)n(C5CCCC5)c4n3)nc2)CC1]

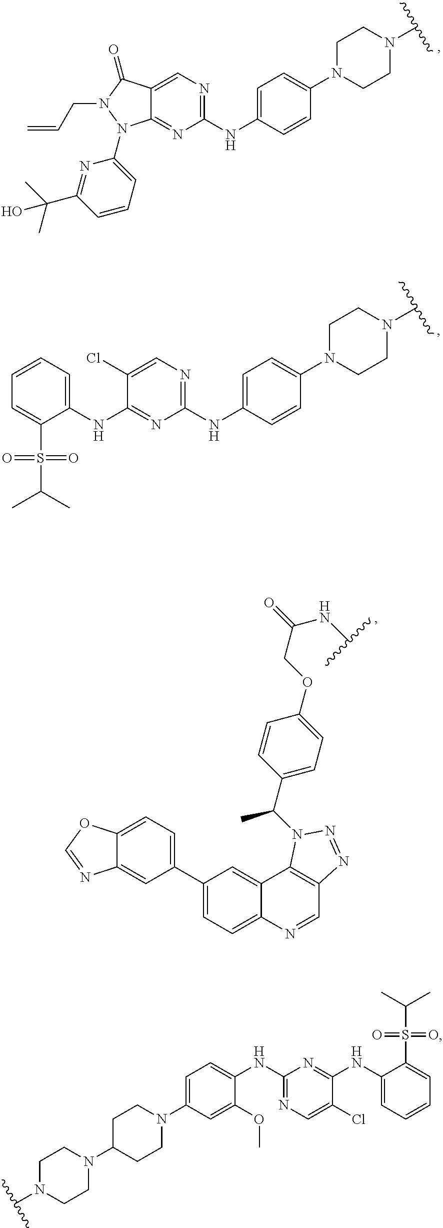 [SMILES: *N1CCN(c2ccc(Nc3ncc4c(=O)n(CC=C)n(-c5cccc(C(C)(C)O)n5)c4n3)cc2)CC1 ; *N1CCN(c2ccc(Nc3ncc(Cl)c(Nc4ccccc4S(=O)(=O)C(C)C)n3)cc2)CC1 ; *NC(=O)COc1ccc([C@H](C)n2nnc3cnc4ccc(-c5ccc6ocnc6c5)cc4c32)cc1 ; *N1CCN(C2CCN(c3ccc(Nc4ncc(Cl)c(Nc5ccccc5S(=O)(=O)C(C)C)n4)c(OC)c3)CC2)CC1]

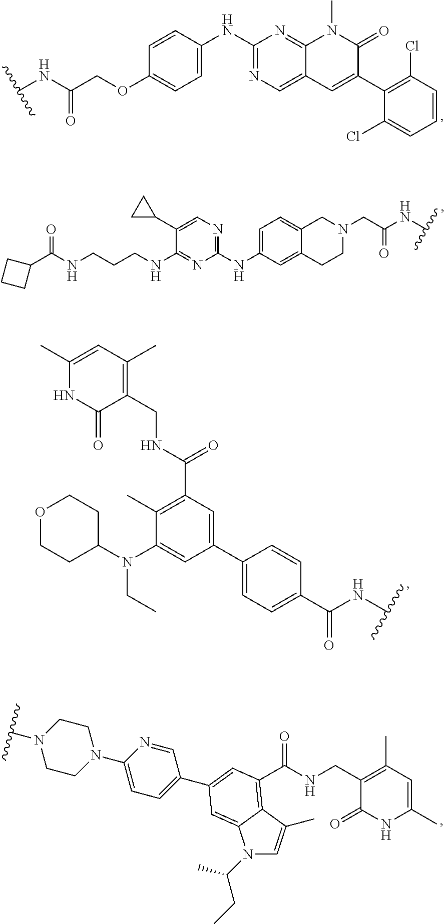 [SMILES: *NC(=O)COc1ccc(Nc2ncc3cc(-c4c(Cl)cccc4Cl)c(=O)n(C)c3n2)cc1 ; *NC(=O)CN1CCc2cc(Nc3ncc(C4CC4)c(NCCCNC(=O)C4CCC4)n3)ccc2C1 ; *NC(=O)c1ccc(-c2cc(C(=O)NCc3c(C)cc(C)[nH]c3=O)c(C)c(N(CC)C3CCOCC3)c2)cc1 ; *N1CCN(c2ccc(-c3cc(C(=O)NCc4c(C)cc(C)[nH]c4=O)c4c(C)cn([C@@H](C)CC)c4c3)cn2)CC1]

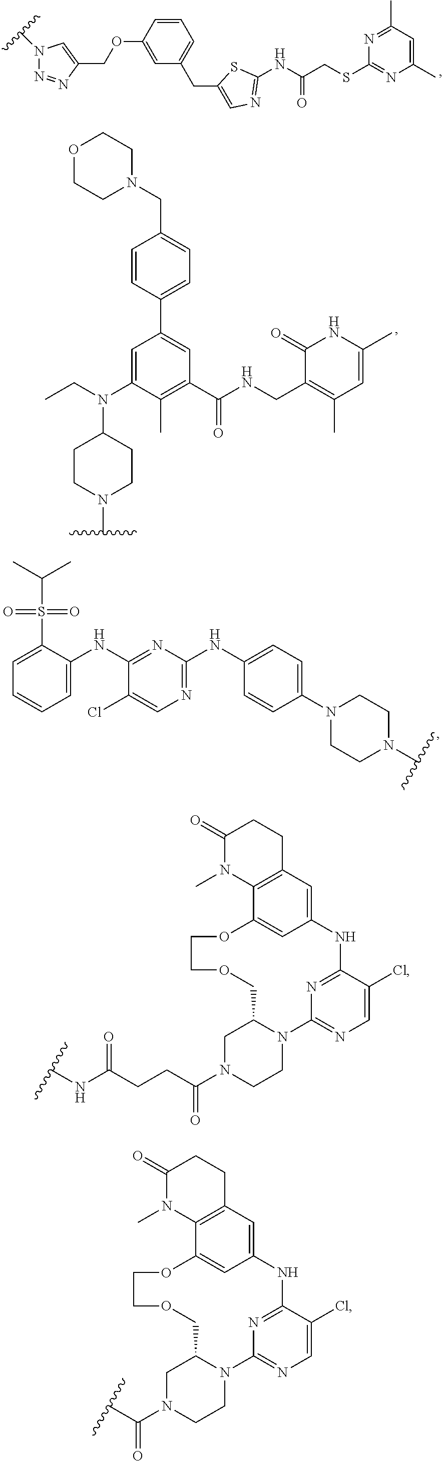

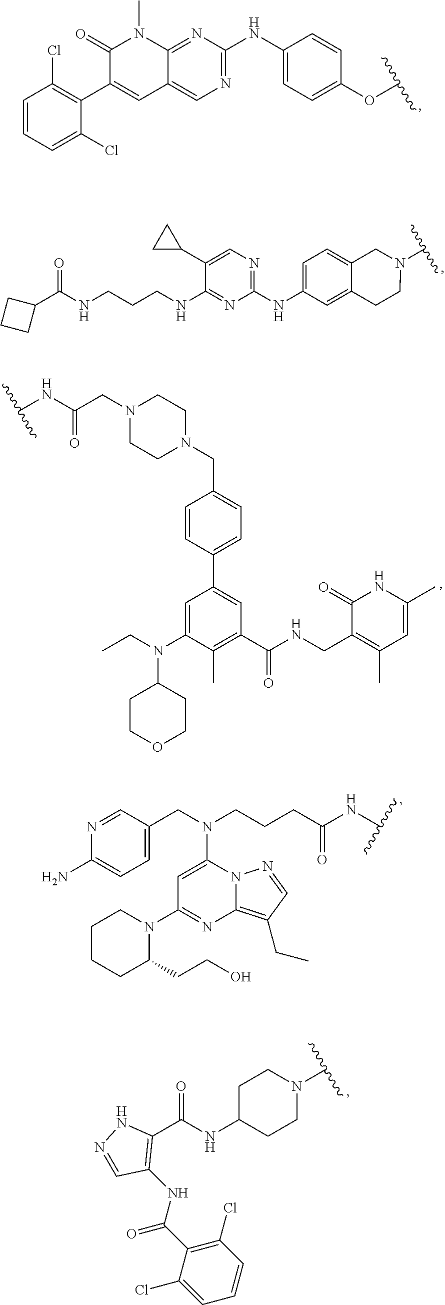 [SMILES: *Oc1ccc(Nc2ncc3cc(-c4c(Cl)cccc4Cl)c(=O)n(C)c3n2)cc1 ; *N1CCc2cc(Nc3ncc(C4CC4)c(NCCCNC(=O)C4CCC4)n3)ccc2C1 ; *NC(=O)CN1CCN(Cc2ccc(-c3cc(C(=O)NCc4c(C)cc(C)[nH]c4=O)c(C)c(N(CC)C4CCOCC4)c3)cc2)CC1 ; *NC(=O)CCCN(Cc1ccc(N)nc1)c1cc(N2CCCC[C@H]2CCO)nc2c(CC)cnn12 ; *N1CCC(NC(=O)c2[nH]ncc2NC(=O)c2c(Cl)cccc2Cl)CC1]

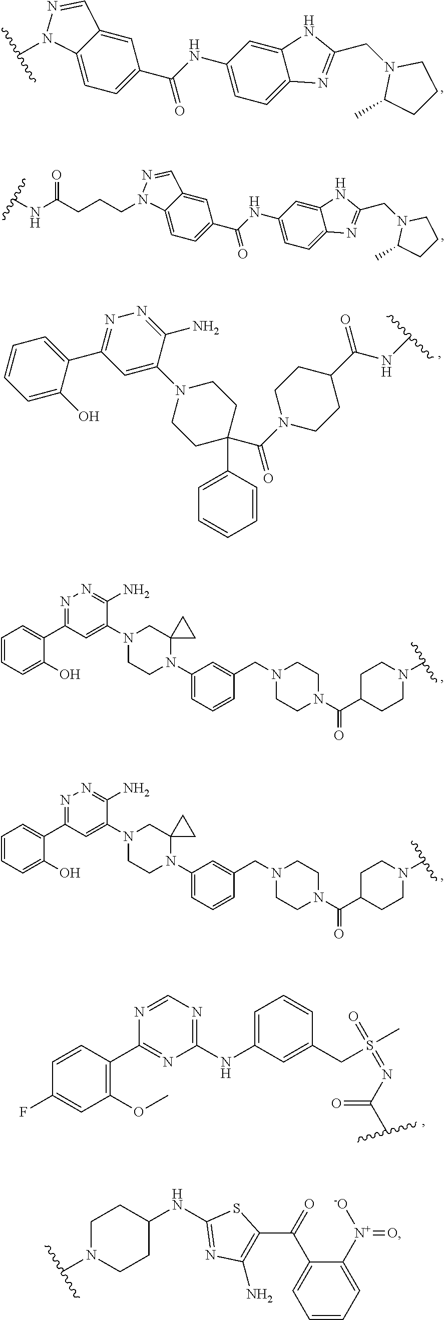

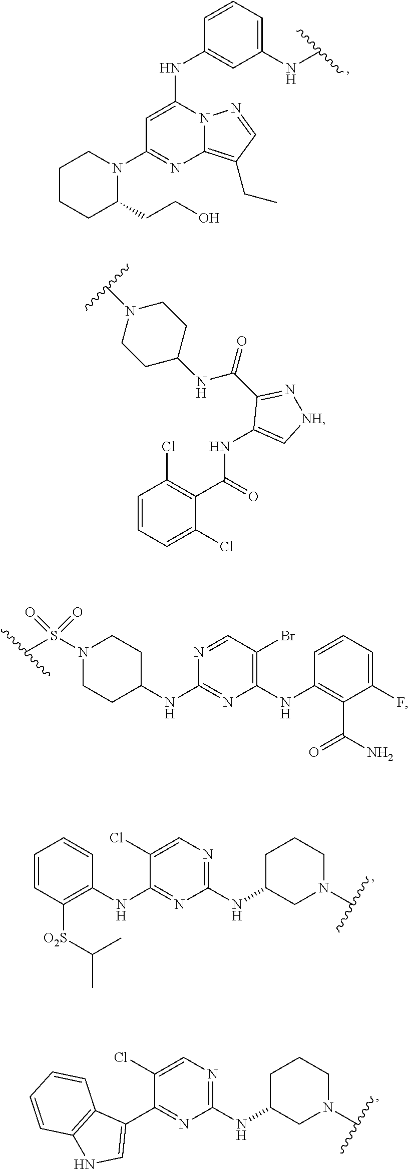

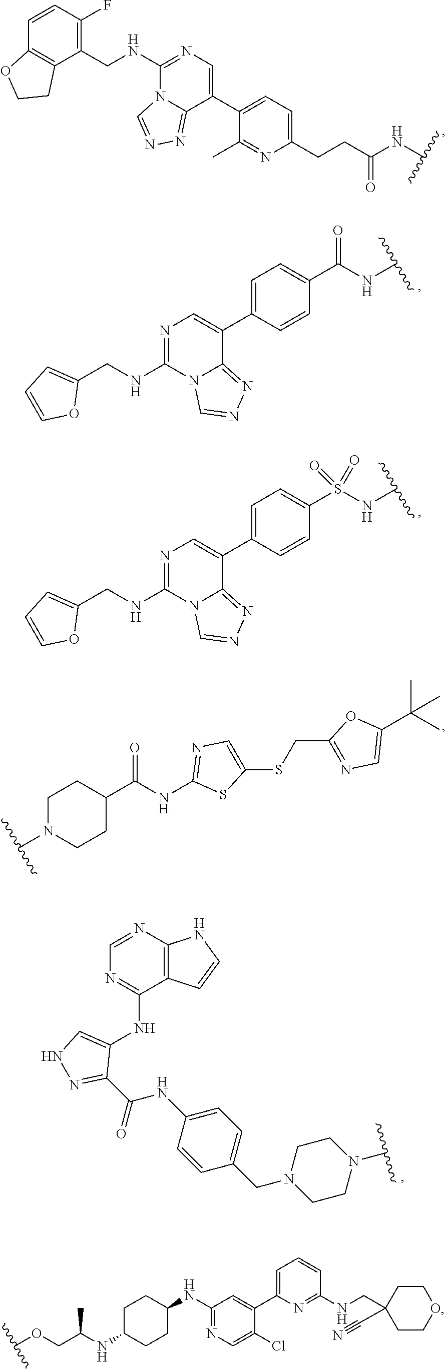 [SMILES: *NC(=O)CCc1ccc(-c2cnc(NCc3c(F)ccc4c3CCO4)n3cnnc23)c(C)n1 ; *NC(=O)c1ccc(-c2cnc(NCc3ccco3)n3cnnc23)cc1 ; *NS(=O)(=O)c1ccc(-c2cnc(NCc3ccco3)n3cnnc23)cc1 ; *N1CCC(C(=O)Nc2ncc(SCc3ncc(C(C)(C)C)o3)s2)CC1 ; *N1CCN(Cc2ccc(NC(=O)c3n[nH]cc3Nc3ncnc4[nH]ccc34)cc2)CC1 ; *OC[C@@H](C)N[C@H]1CC[C@H](Nc2cc(-c3cccc(NCC4(C#N)CCOCC4)n3)c(Cl)cn2)CC1]

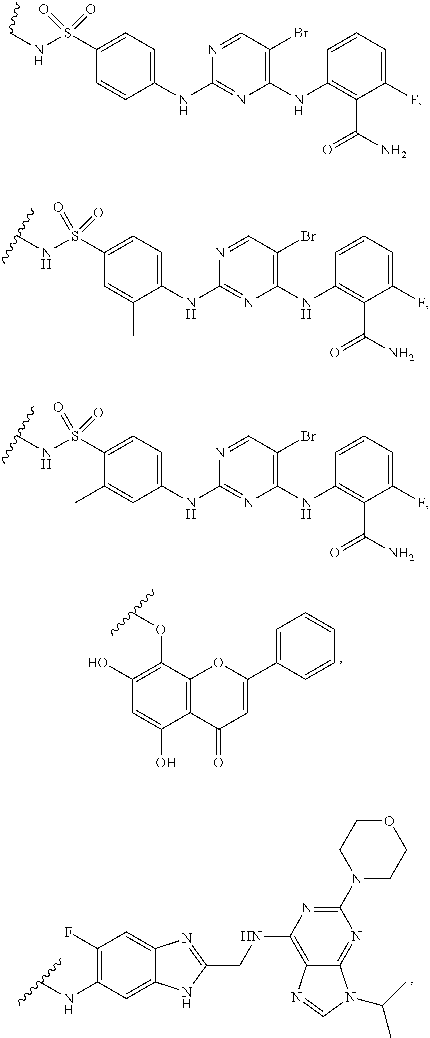

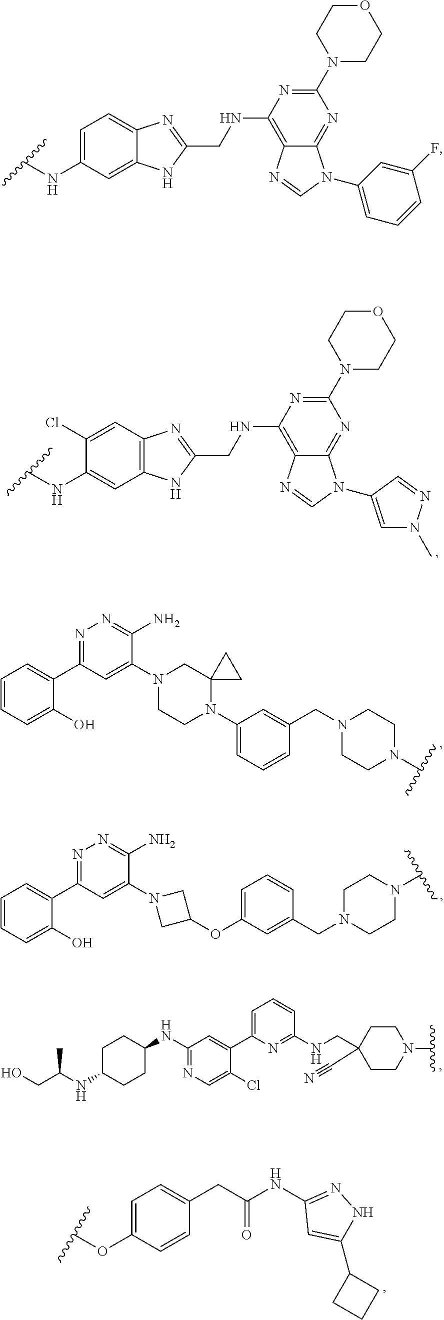 [SMILES: *Nc1ccc2nc(CNc3nc(N4CCOCC4)nc4c3ncn4-c3cccc(F)c3)[nH]c2c1 ; *Nc1cc2[nH]c(CNc3nc(N4CCOCC4)nc4c3ncn4-c3cnn(C)c3)nc2cc1Cl ; *N1CCN(Cc2cccc(N3CCN(c4cc(-c5ccccc5O)nnc4N)CC34CC4)c2)CC1 ; *N1CCN(Cc2cccc(OC3CN(c4cc(-c5ccccc5O)nnc4N)C3)c2)CC1 ; *N1CCC(C#N)(CNc2cccc(-c3cc(N[C@H]4CC[C@H](N[C@H](C)CO)CC4)ncc3Cl)n2)CC1 ; *Oc1ccc(CC(=O)Nc2cc(C3CCC3)[nH]n2)cc1]

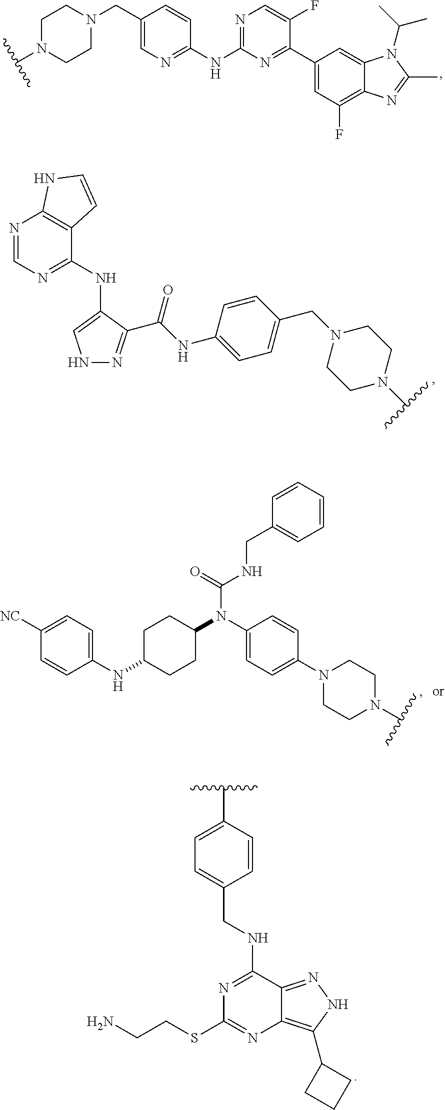

In some embodiments, the binding between the modifier protein and the recruiting ligand comprises a binding affinity with an equilibrium dissociation constant (Kd) below 100 μM, a Kd below 90 μM, a Kd below 80 μM, a Kd below 70 μM, a Kd below 60 μM, a Kd below 50 μM, a Kd below 45 μM, a Kd below 40 μM, a Kd below 35 μM, a Kd below 30 μM, a Kd below 25 μM, a Kd below 20 μM, a Kd below 15 μM, a Kd below 14 μM, a Kd below 13 μM, a Kd below 12 μM, a Kd below 11 μM, a Kd below 10 μM, a Kd below 9 μM, a Kd below 8 μM, a Kd below 7 μM, a Kd below 6 μM, a Kd below 5 μM, a Kd below 4 μM, a Kd below 3 μM, a Kd below 2 μM, or a Kd below 1 μM. In some embodiments, the binding between the modifier protein and the recruiting ligand comprises a binding affinity with an equilibrium dissociation constant (Kd) of at least 100 μM, a Kd of at least 90 μM, a Kd of at least 80 μM, a Kd of at least 70 μM, a Kd of at least 60 μM, a Kd of at least 50 μM, a Kd of at least 45 μM, a Kd of at least 40 μM, a Kd of at least 35 μM, a Kd of at least 30 μM, a Kd of at least 25 μM, a Kd of at least 20 μM, a Kd of at least 15 μM, a Kd of at least 14 μM, a Kd of at least 13 μM, a Kd of at least 12 μM, a Kd of at least 11 μM, a Kd of at least 10 μM, a Kd of at least 9 μM, a Kd of at least 8 μM, a Kd of at least 7 μM, a Kd of at least 6 μM, a Kd of at least 5 μM, a Kd of at least 4 μM, a Kd of at least 3 μM, a Kd of at least 2 μM, or a Kd of at least 1 μM.

Disclosed herein, in some embodiments, are modifier proteins. The modifier protein may modify the target protein. The modifier protein may induce a change in the target protein. For example, the modifier protein may phosphorylate or dephosphorylate the target protein. The modifier protein may exert a catalytic activity upon the target protein.

The modifier protein may include an epigenetic modifier, epigenetic reader, chaperone, protein-protein interaction (PPI) disruptor, mitochondria targeting or nuclear targeting protein.

The modifier protein may include an acylase, deacylase, acetylase, deacetylase, serine/threonine kinase, tyrosine kinase, phosphatase, palmitoyltransferase, methyltransferase, methylase, demethylase, acetyltransferase, deacetylase, glycosyltransferase, SUMO ligase, lyase, hydrolase, isomerase, oxidoreductase, ubiquitin ligase, deubiquitinase, tyrosine sulfotransferase, GTPase, heat shock protein, bromodomain, Tudor domain, PWWP domain, chromodomain, ankyrin repeat, 14-3-3 protein, BRCT domain, DNA recognition protein, DNA modifying protein, or nucleus localization protein. In some embodiments, the modifier protein comprises a functional domain, e.g., a catalytic domain, of the protein provided herein.

The modifier protein may include 14-3-3σ, 14-3-3γ, 14-3-3ε, ABL, AEP1, AhR, ALK, AMPK, AR, ATAD2, ATAD2B, BAZ1A, BAZ1B, BAZ2A, BAZ2B, BCR-ABL, BRAF, BRD2, BRD3, BRD4, BRD7, BRD9, BRDT, BRFA, BRPF1A, BRPF1B, BTK, BRWD3, CBP, CREBBP, CDK2, CDK4, CDK6, CDK7, CDK9, CDK12, CECR2, cIAP, CK1, CRBN, CSF1R, CSN, DCAF11, DCAF15, DCAF16, DOT1, EGFR, ER, EZH2, FAK, FALZ, FEMIB, FKBP12, FLT3, FUT8, G9a, GCN5, GKC, GLP, G9a, HDAC1, HDAC10, HDAC11, HDAC2, HDAC3, HDAC6, HDAC8, HER2, HER4, HSC70, HSP70, HSP90, IGF2R, JAK1, JAK2, JAK3, KAT6A, KAT6B, KAT7, KDM1, KDM2, KDM4, KDM5, KDM6, KEAP1, KIT, KRAS, L3MBTL3, LRRK2, LSD1, LYN, LXR, MAPK, MAX, MEK, MET, MDM2, MLL, MOZ, mTOR, MYC, NBR1, NMT1, NSD2, NSD3, NTRK1, NTRK2, NTRK3, OTUB1, p110, p300, PAX3-FOXO1, PBRM1, PB1, PCAF, PDK1, PDK2, PDGFR, PHID, PHF1, PHF19, PIGK, PI3K, PKC, PKG, PKM2, PKR, PP2A, PP2B, PPP1R15A, PRMT1, PRMT3, PRMT4, PRMT5, PRMT6, PTP1B, Raf-1, RET, RNF4, RNF114, ROCK, ROS1, RPN11, SETD2, SFN, SHP1, SHP2, SIRT1, SIRT3, SIRT6, SMARCA2, SMSARCA4, SMYD2, SMYD3, SOS1, SRC, ST6GAL1, SYK, TAF1, TDRD, Tie2, TOP1, TPST1, TRAF6, TRIM22, TRIM24, TRIM33, TRIM33B, TRIM66, TrkB, TYK2, UAF1, UCHL1, ULK1, USP1, USP7, USP8, USP9X, USP14, USP30, VEGFR1, VEGFR2, VEGFR3, VE-PTP, VHL, WEE1, WRD9/BRWD1, XIAP, YWHAE, YWHAG, ZDHHC11, ZDHHC21, ZDHHC3, ZUP1, ZMYND8, or ZMYND11.

The modifier protein may include an enzyme. Examples of enzymes may include a kinase, phosphatase, palmitoyltransferase, methyltransferase, glycosyltransferase, ligase, hydrolase, acyltransferase, ubiquitin ligase, deubiquitinase or sulfotransferase. In some embodiments, the modifier protein includes a kinase. In some embodiments, the modifier protein includes a phosphatase. The modifier protein may include an alkyltransferase. The modifier protein may include a palmitoyltransferase. The modifier protein may include a methyltransferase. The modifier protein may include a glycosyltransferase. The modifier protein may include a sulfotransferase such as a tyrosine sulfotransferase.

In some embodiments, the modifier protein comprises a palmitoyltransferase, methyltransferase, glycosyltransferase, SUMO ligase, chaperone protein, or tyrosine sulfotransferase. The modifier protein may include a palmitoyltransferase. The modifier protein may include a methyltransferase. The modifier protein may include a glycosyltransferase. The modifier protein may include a SUMO ligase. The modifier protein may include a tyrosine sulfotransferase.

The modifier protein may assist in folding or exert a structural change upon the target protein. For example, the modifier protein may include a chaperone protein.

The modifier protein may include a ligase such as a small ubiquitin-like modifier (SUMO) ligase or an E3 ubiquitin ligase. In some embodiments, the modifier protein comprises an E3 ubiquitin ligase for the inducing activation or other non-degradative effects on the target protein.

An example of a ubiquitin ligase may include VHL. In some embodiments, the modifier protein includes VHL. A ligand included in FIG. 3A or FIG. 3B, or derived therefrom, may be used as a recruiting ligand to bind VHL.

In some embodiments, the modifier protein comprises a deubiquitinase, kinase, methyltransferase, epigenic reader, nuclear localized protein, chaperone or acetyltransferase for activating a tumor suppressor or misfolded protein. In some embodiments, the modifier protein comprises a deubiquitinase. In some embodiments, the modifier protein comprises an acetyltransferase. In some embodiments, the modifier protein comprises an epigenic reader.

An example of a tumor suppressor may include VHL or p53. In some embodiments, the modifier protein includes p53. A ligand included in FIG. 2A or FIG. 2B, or derived therefrom, may be used as a recruiting ligand to bind p53.

In some embodiments, the modifier protein is selected from the list in FIG. 9. In some embodiments, the modifier protein comprises a protein described as an epigenetic reader, an epigenetic modifier, a chaperone, or a DNA intercalators. In some embodiments, the RL directs the target protein directly to DNA using DNA binders as R². In some embodiments, the modifier protein comprises a deacetylase, an acetyltransferase, a ubiquitin iso-peptidase, a pyruvate dehydrogenase kinase, a pyruvate kinase, a tyrosine kinase, a serine/threonine kinase, a fucosyltransferase, histone deacetylase, an autophagy activating kinase, a serine/threonine phosphatase, an enzyme that catalyzes the transfer of sialic acid to galactose, an arginine methyltransferase, an N-terminal methyltransferase, an N-tetradecanoyltransferase, a palmitoyltransferase, a DHHC domain protein, a tyrosylprotein sulfotransferase, a TNF receptor associated factor, a ubiquitin peptidase, a deubiquitylating enzyme, a cytokine cell surface receptor, a cysteine protease, or a leucine-rich repeat kinase.

In some embodiments, the modifier protein comprises a deacetylase, an acetyltransferase, a ubiquitin iso-peptidase, a pyruvate dehydrogenase kinase, a pyruvate kinase, a tyrosine kinase, a serine/threonine kinase, a fucosyltransferase, an autophagy activating kinase, a serine/threonine phosphatase, an enzyme that catalyzes the transfer of sialic acid to galactose, an arginine methyltransferase, an N-terminal methyltransferase, an N-tetradecanoyltransferase, a palmitoyltransferase, a DHHC domain protein, or a tyrosylprotein sulfotransferase. In some embodiments, the modifier protein comprises 5′ AMP-activated protein kinase (AMPK), Sirtuin 1 (SIRT1), CREB binding protein (CBP)/p300, ubiquitin thioesterase OTUB1 (OTUB1), pyruvate dehydrogenase lipoamide kinase isozyme 1,3-phosphoinositide-dependent protein kinase 1 (PDK1), pyruvate kinase muscle isozyme (PKM2), tyrosine-protein kinase ABL1 (c-Abl), alpha-(1,6)-fucosyltransferase (FUT8), protein kinase C (PKC), unc-51 like autophagy activating kinase 1 (ULK1), protein phosphatase 2 (PP2A), beta-galactoside alpha-2,6-sialyltransferase 1 (ST6GAL1), protein arginine N-methyltransferase 5 (PRMT5), glycylpeptide N-tetradecanoyltransferase 1 (NMT1), palmitoyltransferase ZDHHC3 (ZDHHC3), ZDHHC21, ZDHHC11, protein-tyrosine sulfotransferase 1 (TPST1), insulin-like growth factor 2 receptor (IGF2R), or neighbor of BRCA1 gene 1 (NBR1). In some embodiments, the modifier protein comprises AMPK. In some embodiments, the modifier protein comprises an enzyme that deacetylates transcription factors. For example, the modifier protein may include SIRT1 and/or SIRT3. In some embodiments, the modifier protein comprises acetyltransferase activity. For example, the modifier protein may include CBP or p300. In some embodiments, the modifier protein comprises a ubiquitin iso-peptidase. For example, the modifier protein may include OTUB1. In some embodiments, the modifier protein comprises a pyruvate dehydrogenase kinase (PDK) such as PDK1. In some embodiments, the modifier protein comprises a pyruvate kinase such as PKM2. In some embodiments, the modifier protein comprises a tyrosine kinase. Examples of tyrosine kinases include c-Abl, LYN, SYK, FAK, EGFR, PDGFR. In some embodiments, the modifier protein comprises a fucosyltransferase such as FUT8. In some embodiments, the modifier protein comprises a serine/threonine kinase. An example of a serine/threonine kinase may include PKC. In some embodiments, the modifier protein comprises an autophagy activating kinase. An example of an autophagy activating kinase may include ULK1. In some embodiments, the modifier protein comprises a serine/threonine phosphatase. An example of a serine/threonine phosphatase may include PP2A. In some embodiments, the modifier protein catalyzes the transfer of sialic acid to galactose. For example, the modifier protein may include ST6GAL1. In some embodiments, the modifier protein comprises an arginine methyltransferase such as PRMT5. In some embodiments, the modifier protein comprises an N-terminal methyltransferase or N-tetradecanoyltransferase such as NMT1. In some embodiments, the modifier protein comprises a palmitoyltransferase. The palmitoyltransferase may comprise a DHHC domain. Some examples of proteins comprising a DHHC domain may include ZDHHC3, ZDHHC21, or ZDHHC11. In some embodiments, the modifier protein comprises ZDHHC3. In some embodiments, the modifier protein comprises ZDHHC21. In some embodiments, the modifier protein comprises ZDHHC11. In some embodiments, the modifier protein comprises a tyrosylprotein sulfotransferase such as TPST1. In some embodiments, the modifier protein comprises IGF2R. In some embodiments, the modifier protein comprises NBR1.

In some embodiments, the modifier protein comprises a TNF receptor associated factor (TRAF). In some embodiments, the modifier protein comprises TNF receptor associated factor 6 (TRAF6). The TRAF may include TRAF6.

In some embodiments, the modifier protein comprises a ubiquitin peptidase, a deubiquitylating enzyme, a cytokine cell surface receptor, a cysteine protease, or a leucine-rich repeat kinase. In some embodiments, the modifier protein comprises zinc finger containing ubiquitin peptidase 1 (ZUP1), mast/stem cell growth factor receptor Kit (KIT), ubiquitin carboxyl-terminal hydrolase 8 (USP8), Ubiquitin-specific-processing protease 7 (USP7), ubiquitin carboxyl-terminal hydrolase 1 (USP1), GPI-anchor transamidase (PIGK), or leucine-rich repeat kinase 2 (LRRK2). In some embodiments, the modifier protein comprises a zinc finger containing ubiquitin peptidase such as ZUP1. In some embodiments, the modifier protein comprises a cytokine cell surface receptor such as KIT, EGFR, PDGFR. KIT, EGFR and PDGFR may also be examples of tyrosine kinases. In some embodiments, the modifier protein comprises a deubiquitylating enzyme. Examples of deubiquitylating enzymes may include USP1, USP7, USP8, USP9X, USP30, UCHL1, CSN, USP1/UAF1, or RPN11. In some embodiments, the modifier protein comprises USP8. In some embodiments, the modifier protein comprises USP7. In some embodiments, the modifier protein comprises USP1. In some embodiments, the modifier protein comprises a cysteine protease such as a member of the cysteine protease family C13. For example, the modifier protein may include PIGK. In some embodiments, the modifier protein comprises a leucine-rich repeat kinase such as LRRK2.

In some embodiments, the modifier protein comprises AMPK, SIRT1, SIRT3, CBP/p300, OTUB1, PDK1, PKM2, c-Abl, PKC, ULK1, or PP2A.

In some embodiments, the modifier protein includes an FKBP such as FKBP12.

The modifier protein may be in a subject. The modifier protein may be in a cell. The modifier protein may be intrinsic to the cell. The modifier protein may be endogenous to the cell. In some embodiments, the modifier protein is extrinsic to the cell. In some embodiments, the modifier protein is exogenous to the cell. The cell may be a cell of the subject.

In some embodiments, the modifier protein induces a change to the target protein. For example, the modifier protein may modify the target protein or induce a structural change. The modification may include changes in cellular localization. The modification may be an enzymatic modification. The modification may include a post-translational modification. Some examples of modifications may include phosphorylation, palmitoylation, methylation, glycosylation, ligation, sulfonation, ubiquitylation, SUMOylation, fucosylation, sialation, tyrosylation, acylation, acetylation, or tetradecanoylation. The modification may be added to the target protein by the modifier protein. The modification may be removed from the target protein by the modifier protein. For example, a phosphate group may be added to the target protein (e.g. phosphorylation or ubiquitylation) or removed (e.g. dephosphorylation or deubiquitylation) from the target protein. The modification may include phosphorylation, dephosphorylation, methylation, demethylation, glycosylation, deglycosylation, ubiquitylation, or deubiquitylation. The modification may be covalent or non-covalent. Examples of structural changes may include protein folding, misfolding, or cleavage. The modification may be the change of cellular localization.

In some embodiments, the modifier protein comprises a bromodomain-containing protein (BRD). In some embodiments, the bromodomain-containing protein (BRD) comprises of a multidomain protein. The BRD-containing proteins comprises a combination of protein-protein interaction domains, such as the plant homeodomain (PHD), KID-interacting domain (KIX) and bromo-adjacent homology domain (BAH), with catalytic domains, such as histone acetyltransferase (HAT) and helicase domains. The existence of such combinations of functional modules within each protein facilitates interactions with other proteins within complexes, recruitment to specific sites and directed catalytic activity.

In some embodiments, the modifier protein comprises of P300/CBP-associated factor (PCAF), bromodomain-containing protein (BRD2, BRD3, BRD4), CREB-binding protein (CREBBP), bromodomain and PHD finger-containing protein 1 (BRPF1), tripartite motif-containing proteins (TRIM22, TRIM24, TRIM33), speckled 140 kDa (SP140), zinc finger MYND-type containing 8 (ZMYND8), polybromo 1 (PB1) and SWI/SNF-related, matrix-associated, actin-dependent regulator of chromatin subfamily A members (SMARCA2, SMARCA4). In some embodiments, the modifier protein comprises BRD2. In some embodiments, the modifier protein comprises BRD3. In some embodiments, the modifier protein comprises BRD4. In some embodiments, the modifier protein comprises of ATPase family AAA domain-containing protein 2 (ATAD2), bromodomain adjacent to zinc finger domain 1A (BAZ1A,), bromodomain and PHD finger-containing transcription factors (BPTF); BRD testis-specific protein (BRDT), cat eye syndrome critical region protein 2 (CECR2), cAMP-responsive element-binding protein (CREB), Lysine Acetyltransferase 2A (KAT2A/GCN5), transcription initiation factor TFIID subunit 1 (TAF1), TAF1-like protein (TAF1L), transcription adaptor putative zinc finger (TAZ).

### Linkers

Disclosed herein, in some embodiments, are linkers. The linker may be a part of a heterobifunctional compound such as an OmniTAC compound. The linker may connect a targeting ligand to a recruiting ligand. The linker may directly connect to both the targeting ligand and the recruiting ligand. The linker may include a ligand of a protein such as a target protein.

In some embodiments, the linker comprises a polyethylene glycol. In some embodiments, the linker comprises an aromatic group. In some embodiments, the linker comprises an alkyl chain. In some embodiments, the linker comprises an alkenyl. In some embodiments, the linker comprises an alkyl phosphate. In some embodiments, the linker comprises an alkyl siloxane. In some embodiments, the linker comprises an epoxy. In some embodiments, the linker comprises an acrylamide. In some embodiments, the linker comprises an N-acyl-N-alkyl sulfonamide. In some embodiments, the linker comprises an oxaziridine. In some embodiments, the linker comprises a glycidyl. In some embodiments, the linker comprises a carboxylate. In some embodiments, the linker comprises an anhydride. In some embodiments, the linker comprises a polypeptide. In some embodiments, the linker comprises piperazine. In some embodiments, the linker comprises piperidyl. In some embodiments, the linker comprises triazole.

In some embodiments, the linker comprises a chain length of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, or a range defined by any two of the aforementioned numbers of atoms. In some embodiments, the linker comprises a chain length of between 2 to 24 atoms. In some embodiments, the linker comprises a chain length of between 2 to 18 atoms.

In some embodiments, the linker comprises: (a) a structure selected from the non-limiting group consisting of polyethylene glycol, an aromatic group, an alkyl, an alkenyl, an alkyl phosphate, an alkyl siloxane, an epoxy, a glycidyl, a carboxylate, an anhydride, a piperazine, a piperidyl, a triazole, or a combination thereof; or (b) a polypeptide of natural or synthetic source having a chain length of between 2 to 24 amino acids.

In some embodiments, the linker comprises one or more covalently connected structural units of A (e.g., -A₁ . . . A_{q}-), wherein A, is a group coupled to at least one of a recruiting ligand, a targeting ligand, or a combination thereof. In some embodiments, A₁ links a recruiting ligand, a targeting ligand, or a combination thereof directly to another recruiting ligand, targeting ligand or combination thereof. In some embodiments, A₁ links a recruiting ligand, a targeting ligand, or a combination thereof indirectly to another recruiting ligand, targeting ligand, or combination thereof through A_{q}.

In some embodiments, A₁ to A_{q} are, each independently, a bond, CR^{L1}R^{L2}, O, S, SO, SO₂, NR^{L3}, SO₂NR^{L3}, SONR^{L3}, CONR^{L3}, NR^{L3}CONR^{L4}, NR^{L3}SO₂NR^{L4}, CO, CR^{L4}═CR^{L2}, C≡C, SiR^{L1}R^{L2}, P(O)R^{L1}, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} or R^{L2} groups, C₃₋₁₁heterocyclyl optionally substituted with 0-6 R^{L1} or R^{L2} groups, aryl optionally substituted with 0-6 R^{L1} or R^{L2} groups, heteroaryl optionally substituted with 0-6 R^{L1} or R^{L2} groups, where R^{L1} or R^{L2}, each independently, can be linked to other A groups to form cycloalkyl or heterocyclyl moiety which can be further substituted with 0-4 R^{L5} groups. In some embodiments, R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁ cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈ cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈ alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl) SO₂N(C₁₋₈alkyl)₂, NH SO₂NH(C₁₋₈alkyl), NH SO₂N(C₁₋₈alkyl)₂. NH SO₂NH₂. In some embodiments. q is an integer greater than or equal to 0. In some embodiments, q is an integer greater than or equal to 1. In some embodiments. e.g., where q is greater than 2, A_{q} is a group which is connected to a recruiting ligand or recruiting ligand moiety, and A₁ and A_{q} are connected via structural units of A (number of such structural units of A: q-2). In some embodiments, e.g., where q is 2, A_{q} is a group which is connected to A₁ and to a recruiting ligand or recruiting ligand moiety. In some embodiments, e.g., where q is 1, the structure of the linker is -A₁-, and A₁ is a group which is connected to a recruiting ligand or recruiting ligand moiety and a targeting ligand moiety. In some embodiments, q is an integer from 1 to 100, 1 to 90, 1 to 80, 1 to 70, 1 to 60, 1 to 50, 1 to 40, 1 to 30, 1 to 20, or 1 to 10.

In some embodiments, the linker is selected from any of the following linker examples:

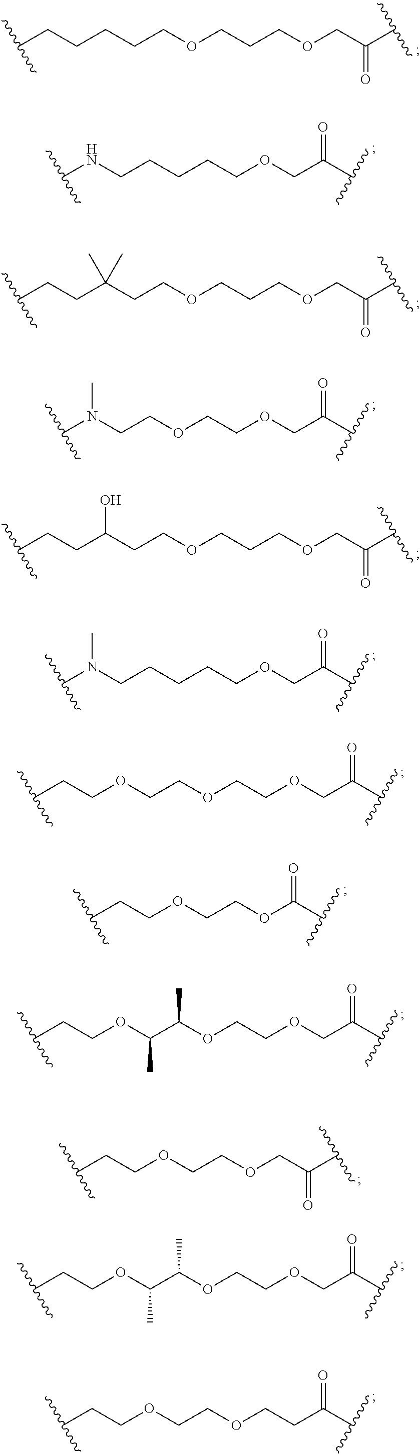

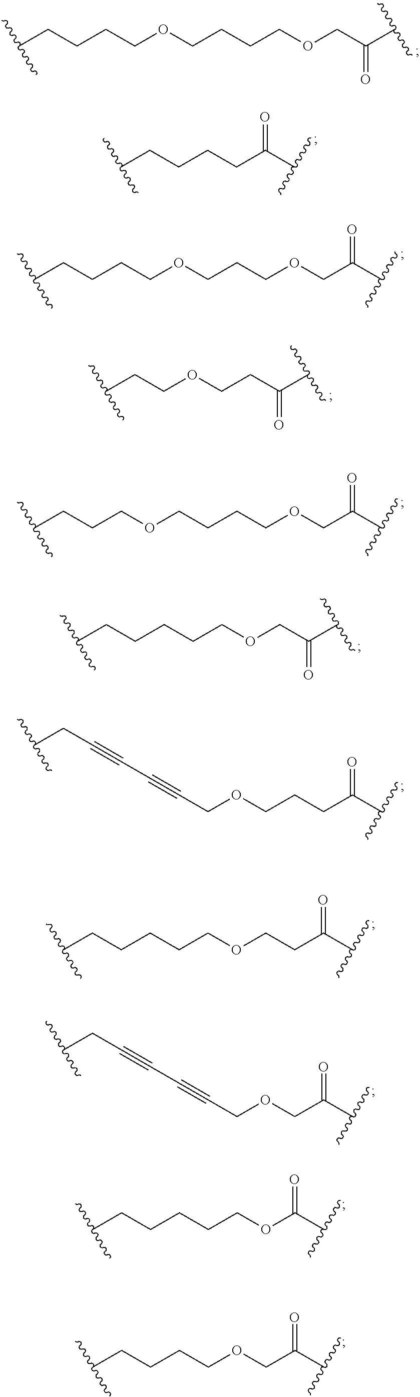

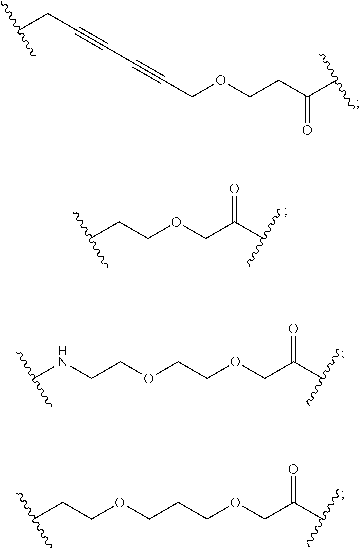 [SMILES: *CC#CC#CCOCCC(*)=O ; *CCOCC(*)=O ; *NCCOCCOCC(*)=O ; *CCOCCCOCC(*)=O]

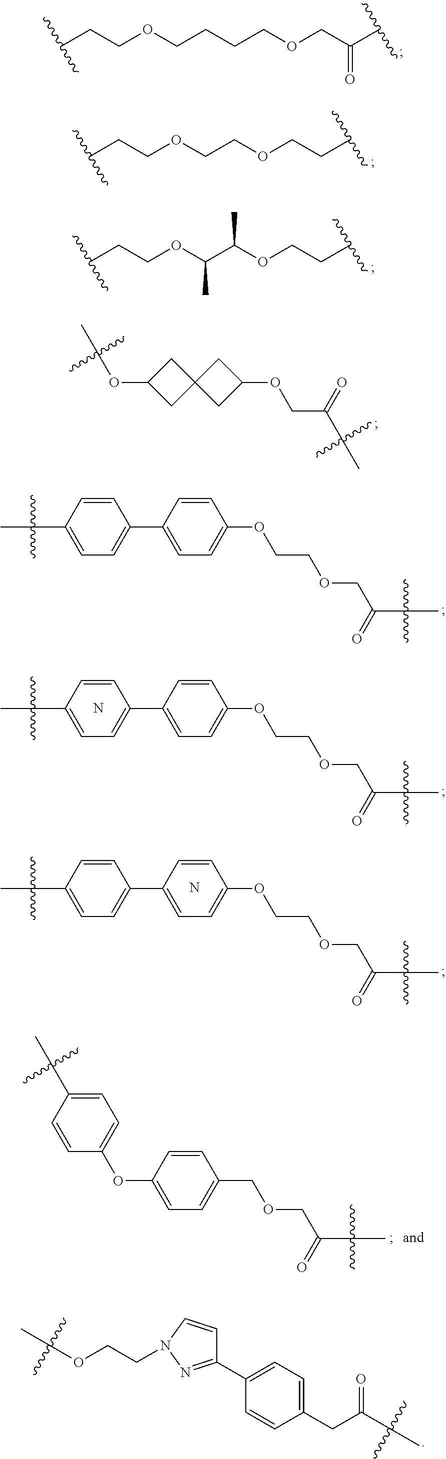

In some embodiments, the linker is selected from any of the following linker example:

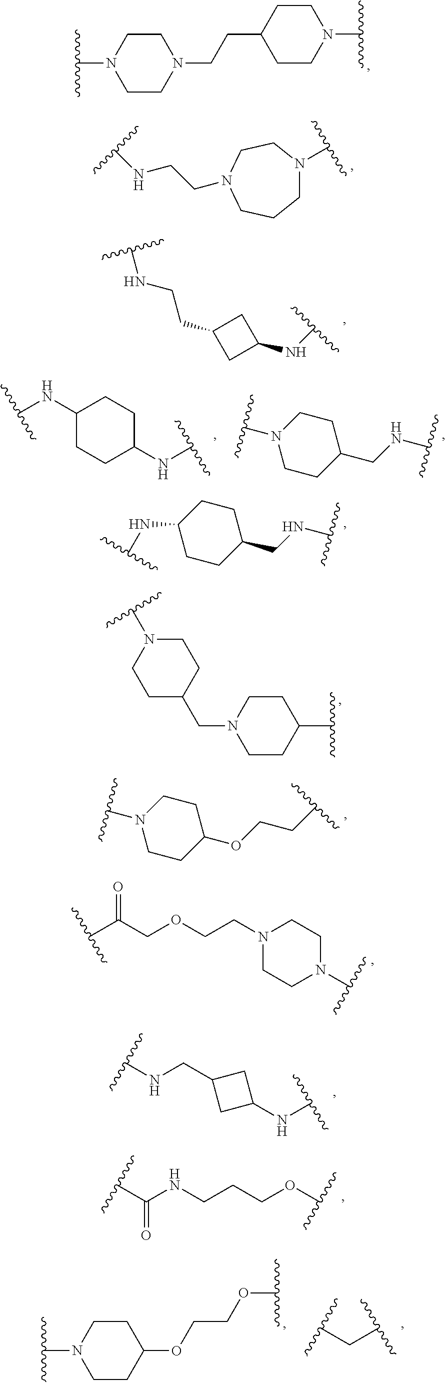

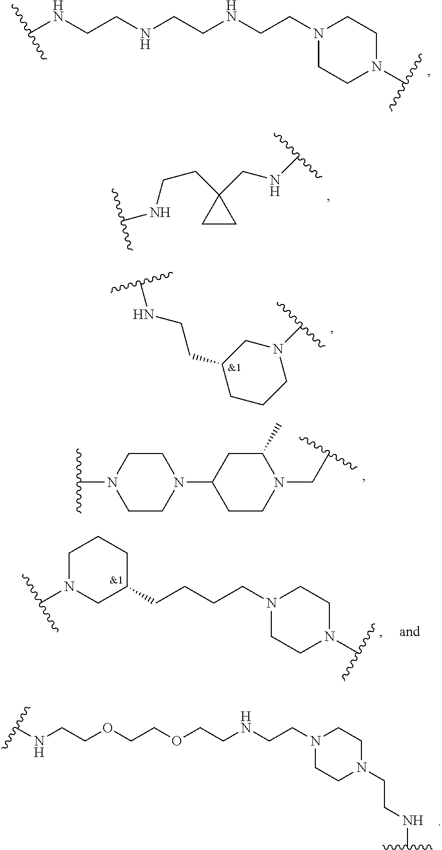

In some embodiments, the linker comprises or consists of optionally substituted (poly)ethyleneglycol having about 1 to about 100 ethylene glycol units, about 1 to about 50 ethylene glycol units, about 1 to about 25 ethylene glycol units, about 1 to about 10 ethylene glycol units, about 1 to about 8 ethylene glycol units, about 1 to about 6 ethylene glycol units, about 2 to about 4 ethylene glycol units, or optionally substituted alkyl groups interspersed with optionally substituted, O, N, S, P or Si atoms. In some embodiments, the linker is substituted with an aryl, phenyl, benzyl, alkyl, alkylene, halogen, or heterocycle group. In some embodiments, the linker may be asymmetric or symmetrical.

In any of the embodiments of the compounds described herein, the linker may be any suitable moiety as described herein. In some embodiments, the linker is or includes a substituted or unsubstituted polyethylene glycol group ranging in size from about 1 to about 12 ethylene glycol units, about 1 to about 10 ethylene glycol units, about 2 to about 6 ethylene glycol units, about 2 to 5 ethylene glycol units, and about 2 to about 4 ethylene glycol units.

In some embodiments, the linkers can have various attachment points to the recruiting ligand. For example, the linkers can attach to the recruitment ligand via an amide or triazole at different points. In a non-limiting illustrative example, the three structures below illustrate how the recruiting ligand on the right side of the molecule attaches via an amide or triazole functional group to the linker:

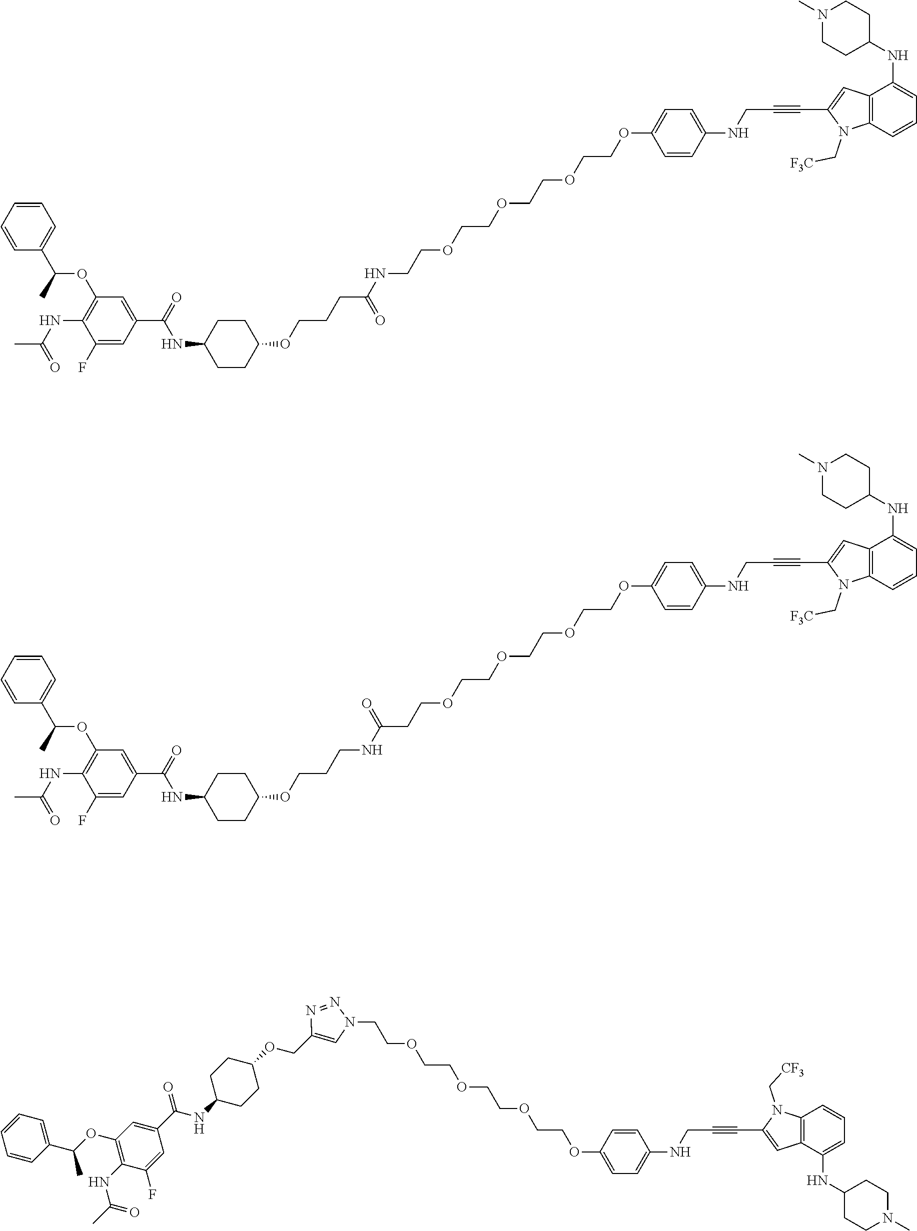 [SMILES: CC(=O)Nc1c(F)cc(C(=O)N[C@H]2CC[C@H](OCCCC(=O)NCCOCCOCCOCCOc3ccc(NCC#Cc4cc5c(NC6CCN(C)CC6)cccc5n4CC(F)(F)F)cc3)CC2)cc1O[C@@H](C)c1ccccc1 ; CC(=O)Nc1c(F)cc(C(=O)N[C@H]2CC[C@H](OCCCNC(=O)CCOCCOCCOCCOc3ccc(NCC#Cc4cc5c(NC6CCN(C)CC6)cccc5n4CC(F)(F)F)cc3)CC2)cc1O[C@@H](C)c1ccccc1 ; CC(=O)Nc1c(F)cc(C(=O)N[C@H]2CC[C@H](OCc3cn(CCOCCOCCOCCOc4ccc(NCC#Cc5cc6c(NC7CCN(C)CC7)cccc6n5CC(F)(F)F)cc4)nn3)CC2)cc1O[C@@H](C)c1ccccc1]

Although the recruiting ligand and targeting ligand may be covalently linked to the linker through any group which is appropriate and stable to the chemistry of the linker, in some embodiments, the linker may be independently covalently bonded to the recruiting ligand and the targeting ligand, for example through an amide, ester, thioester, sulfone, sulfonamide, phosphinoxide, keto group, amine, carbamate (urethane), urea, carbon or ether, each of which groups may be inserted anywhere on the recruiting ligand and targeting ligand to provide maximum binding of a recruiting ligand on a modifier protein and a targeting ligand on a target protein. In some embodiments, the linker may be linked to an optionally substituted alkyl, alkylene, alkene or alkyne group, an aryl group or a heterocyclic group on a recruiting ligand or a targeting ligand.

In some embodiments, examples of a targeting ligand and linker include but is not limited to:

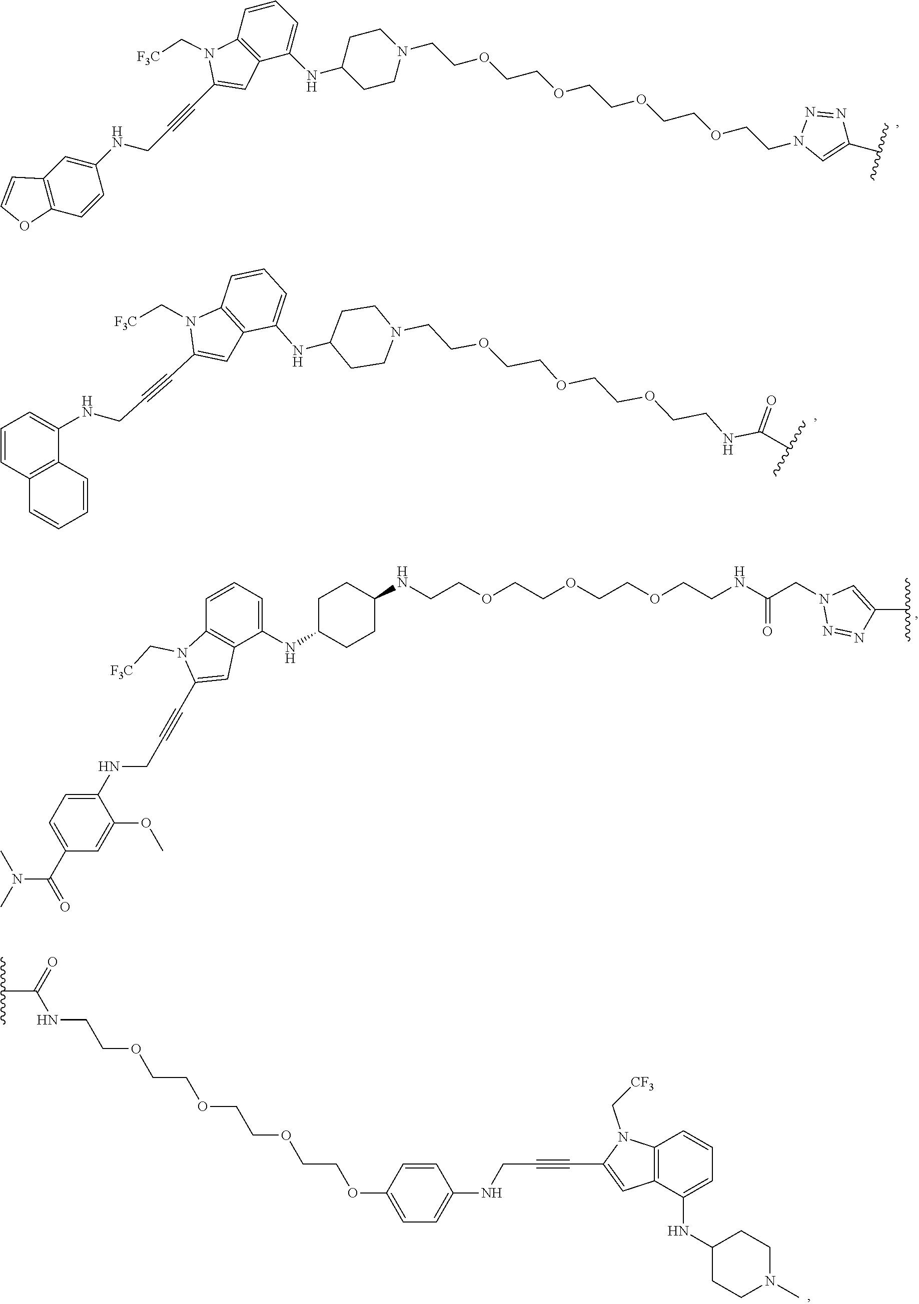 [SMILES: *c1cn(CCOCCOCCOCCOCCN2CCC(Nc3cccc4c3cc(C#CCNc3ccc5occc5c3)n4CC(F)(F)F)CC2)nn1 ; *C(=O)NCCOCCOCCOCCN1CCC(Nc2cccc3c2cc(C#CCNc2cccc4ccccc24)n3CC(F)(F)F)CC1 ; *c1cn(CC(=O)NCCOCCOCCOCCN[C@H]2CC[C@H](Nc3cccc4c3cc(C#CCNc3ccc(C(=O)N(C)C)cc3OC)n4CC(F)(F)F)CC2)nn1 ; *C(=O)NCCOCCOCCOCCOc1ccc(NCC#Cc2cc3c(NC4CCN(C)CC4)cccc3n2CC(F)(F)F)cc1]

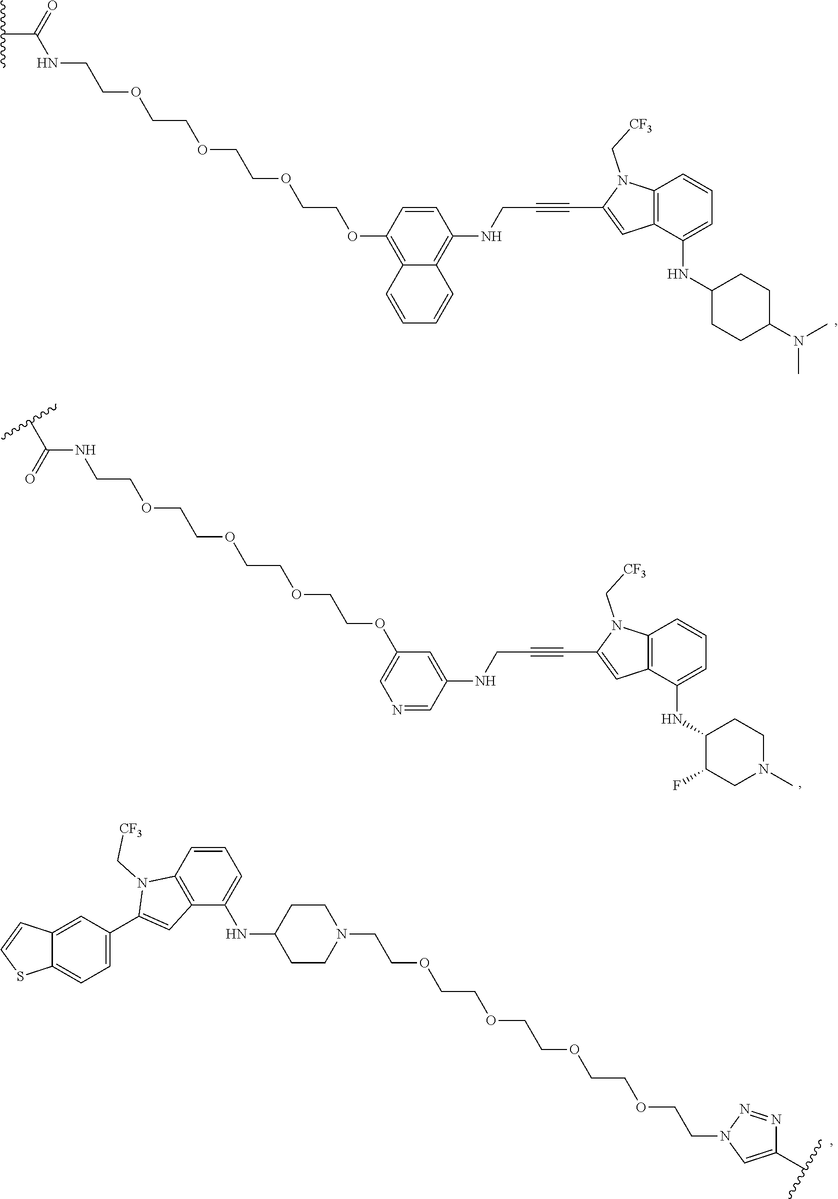 [SMILES: *C(=O)NCCOCCOCCOCCOc1ccc(NCC#Cc2cc3c(NC4CCC(N(C)C)CC4)cccc3n2CC(F)(F)F)c2ccccc12 ; *C(=O)NCCOCCOCCOCCOc1cncc(NCC#Cc2cc3c(N[C@@H]4CCN(C)C[C@@H]4F)cccc3n2CC(F)(F)F)c1 ; *c1cn(CCOCCOCCOCCOCCN2CCC(Nc3cccc4c3cc(-c3ccc5sccc5c3)n4CC(F)(F)F)CC2)nn1]

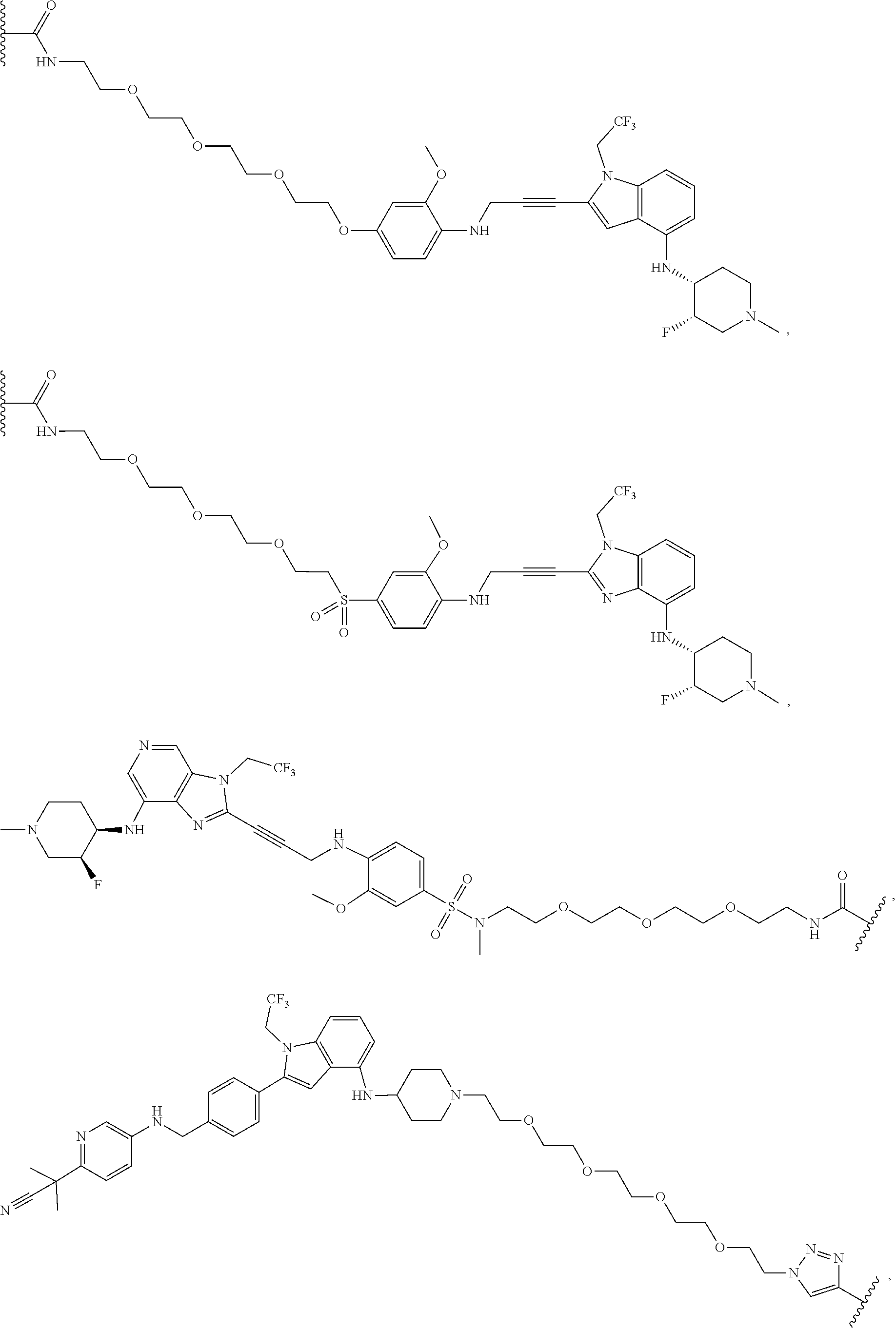

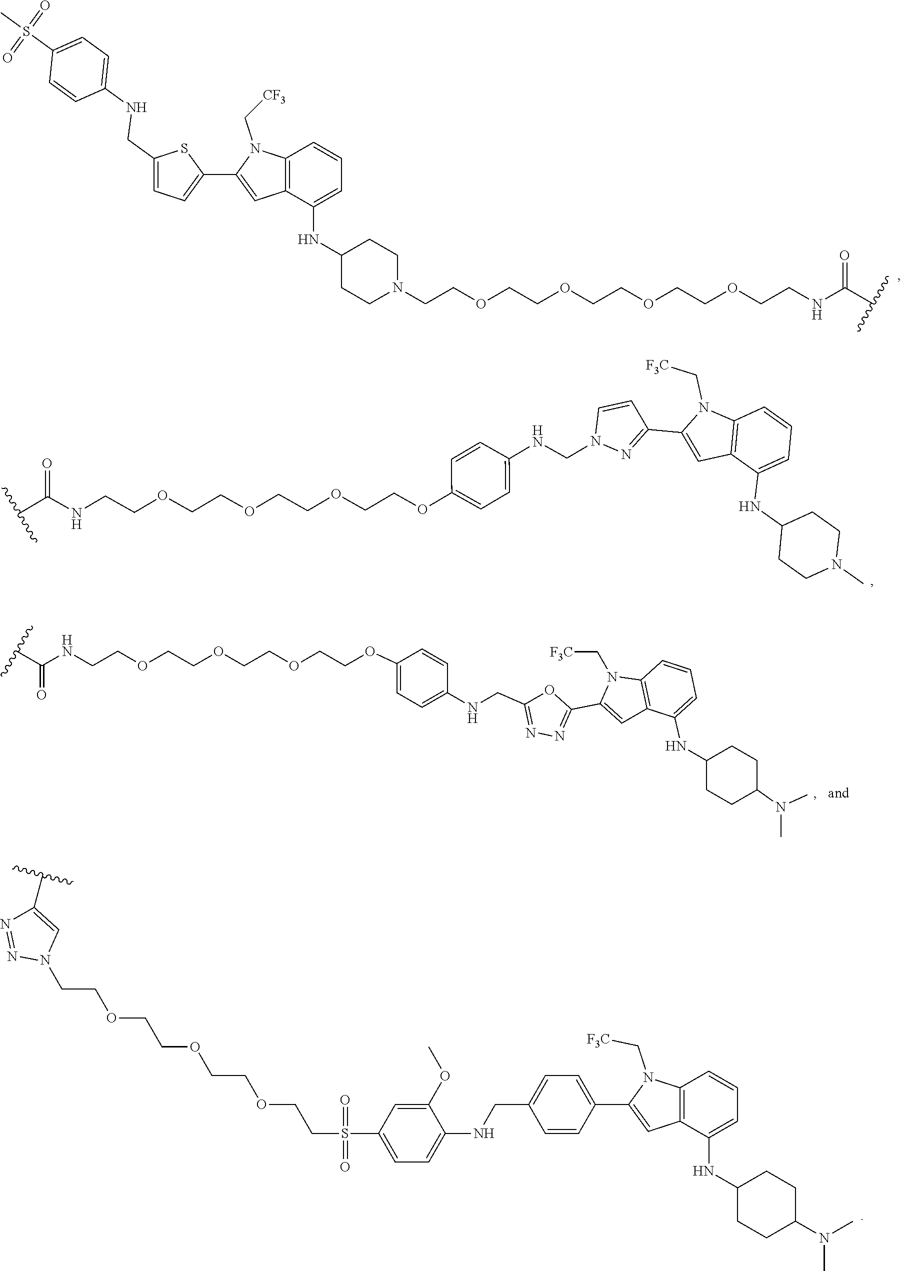 [SMILES: *C(=O)NCCOCCOCCOCCOCCN1CCC(Nc2cccc3c2cc(-c2ccc(CNc4ccc(S(C)(=O)=O)cc4)s2)n3CC(F)(F)F)CC1 ; *C(=O)NCCOCCOCCOCCOc1ccc(NCn2ccc(-c3cc4c(NC5CCN(C)CC5)cccc4n3CC(F)(F)F)n2)cc1 ; *C(=O)NCCOCCOCCOCCOc1ccc(NCc2nnc(-c3cc4c(NC5CCC(N(C)C)CC5)cccc4n3CC(F)(F)F)o2)cc1 ; *c1cn(CCOCCOCCOCCS(=O)(=O)c2ccc(NCc3ccc(-c4cc5c(NC6CCC(N(C)C)CC6)cccc5n4CC(F)(F)F)cc3)c(OC)c2)nn1]

In some embodiments, the linker is:

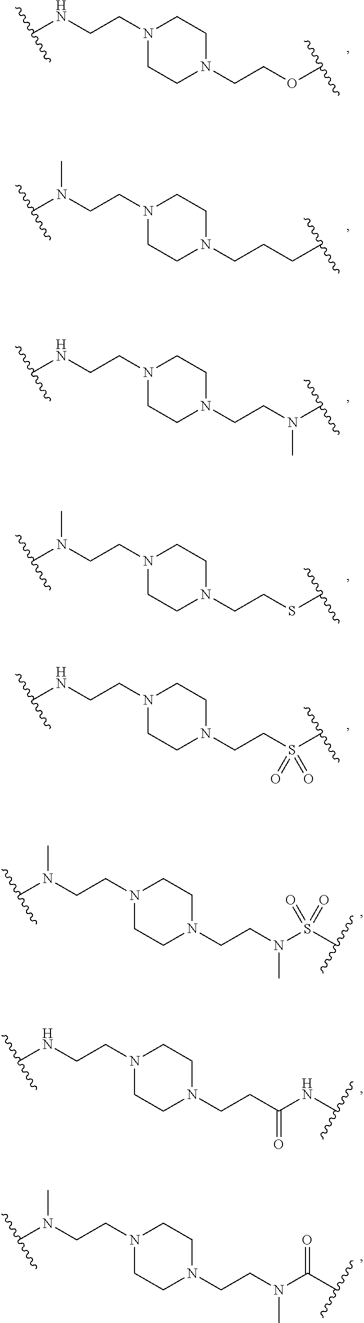

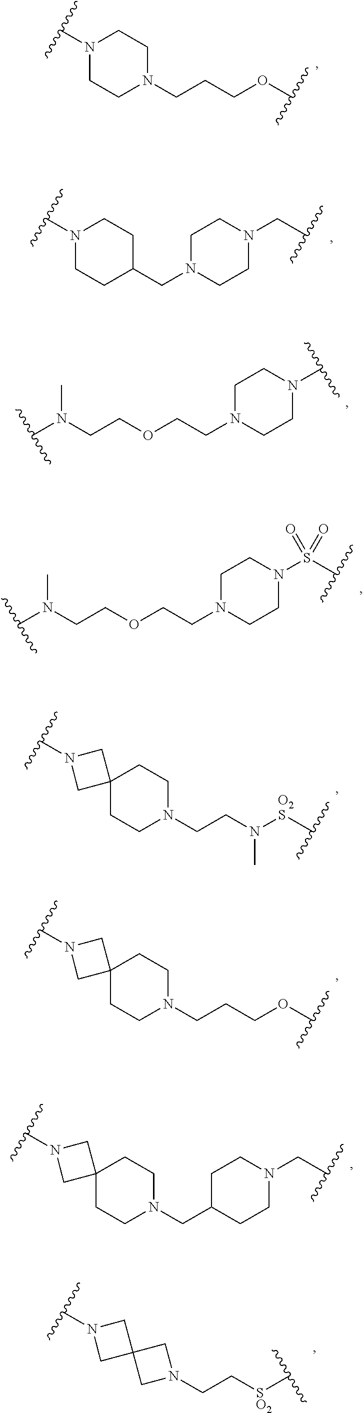

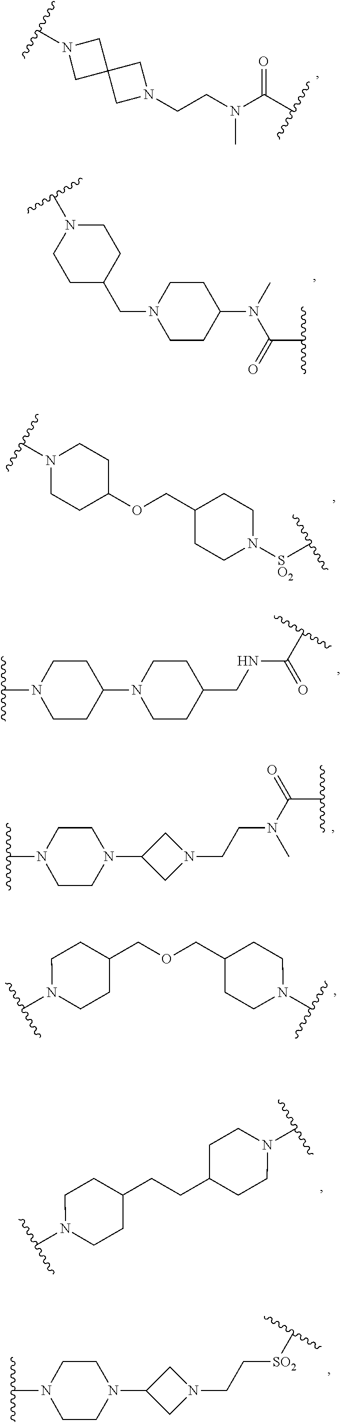

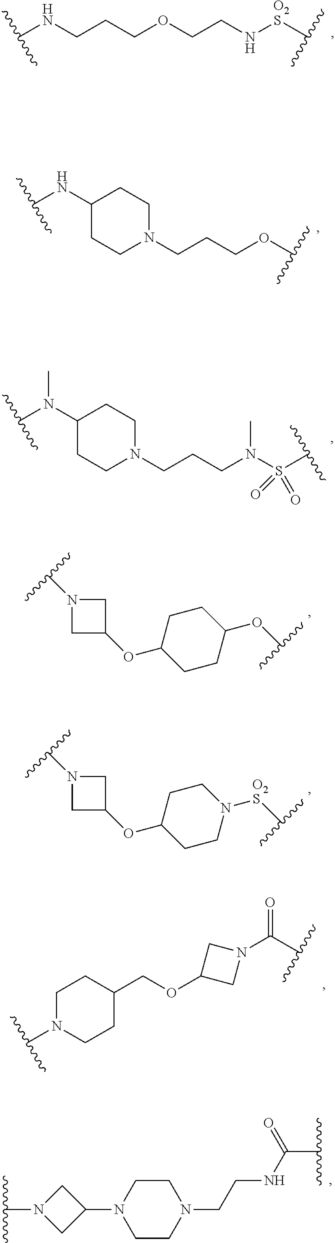

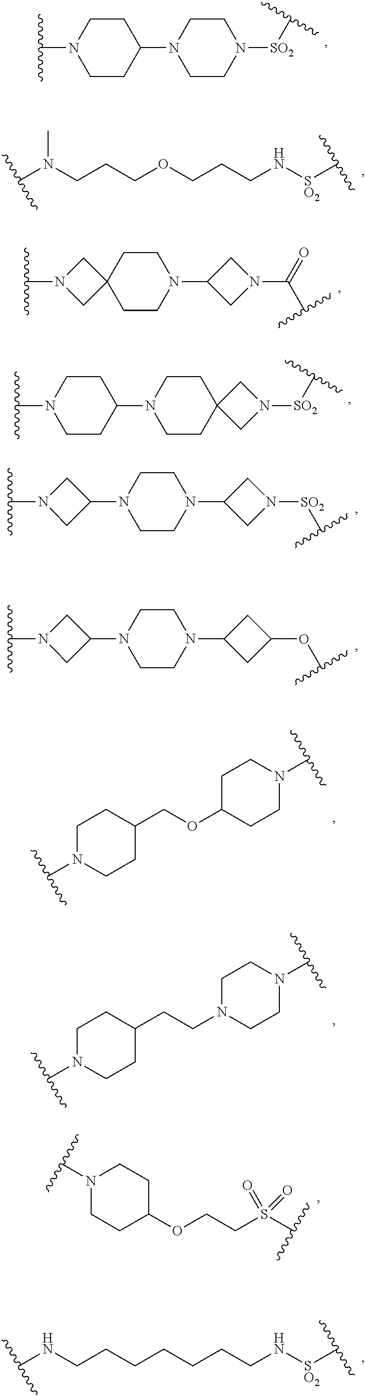

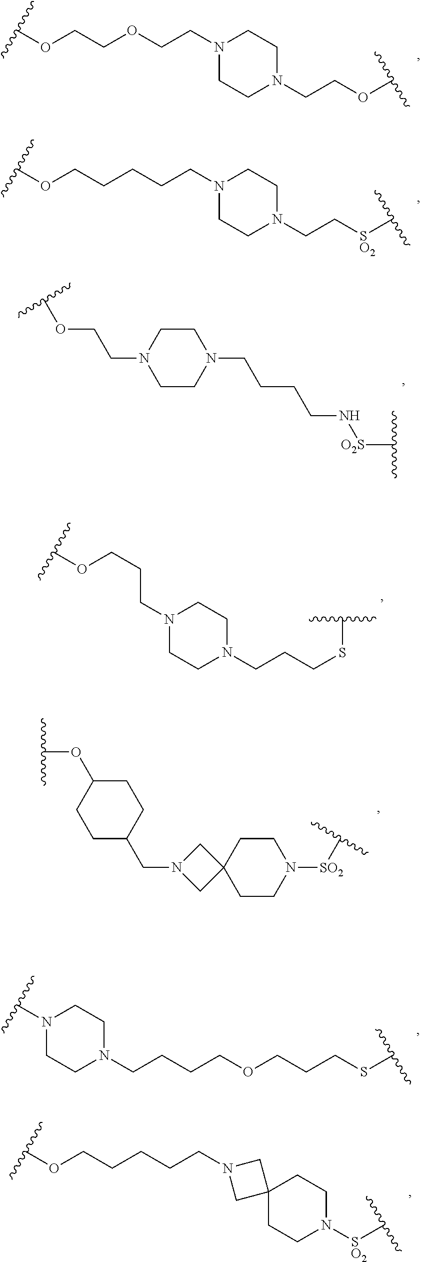

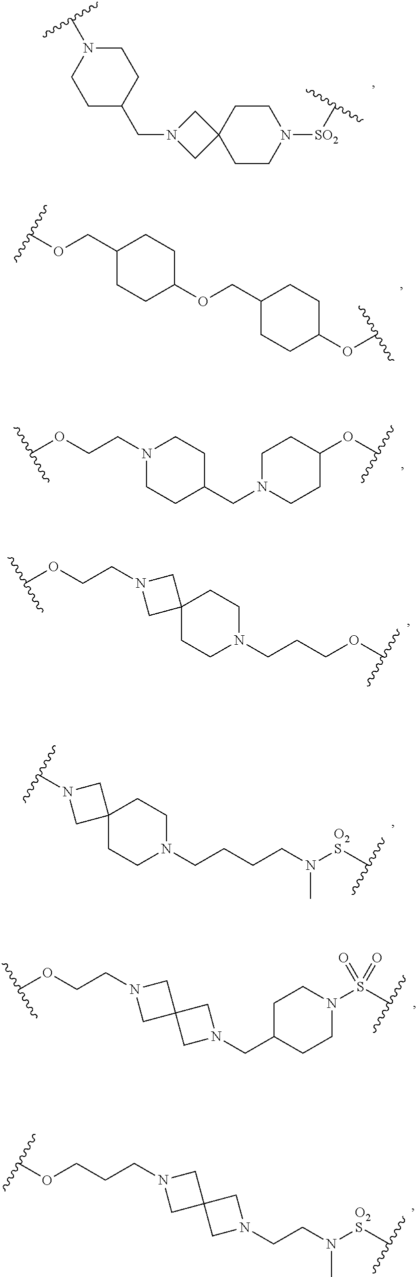

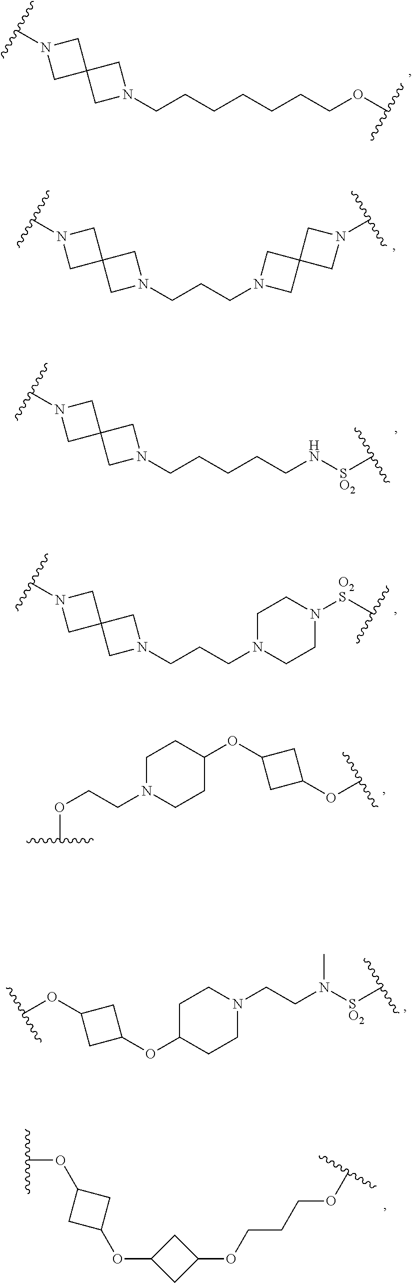

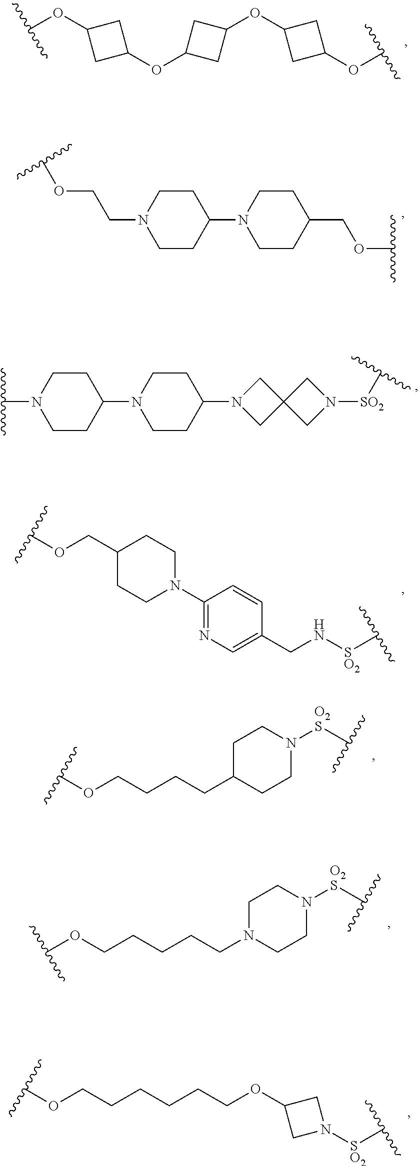

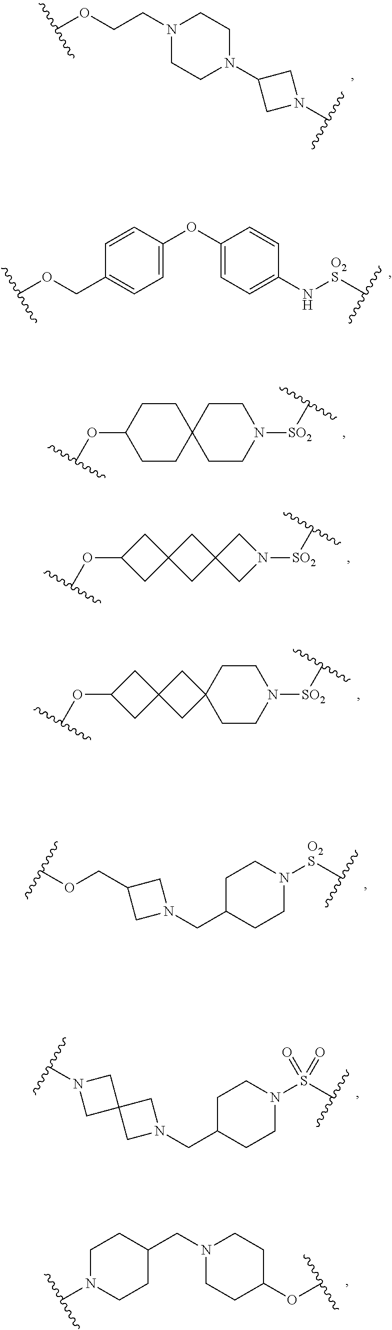

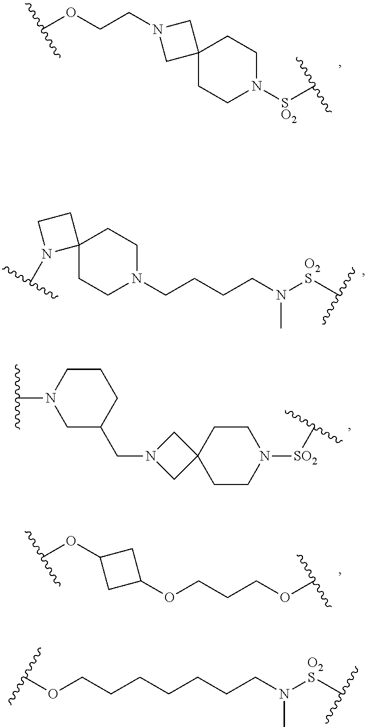 [SMILES: *OCCN1CC2(CCN(S(*)(=O)=O)CC2)C1 ; *N1CCC12CCN(CCCCN(C)S(*)(=O)=O)CC2 ; *N1CCCC(CN2CC3(CCN(S(*)(=O)=O)CC3)C2)C1 ; *OCCCOC1CC(O*)C1 ; *OCCCCCCCN(C)S(*)(=O)=O]

or any suitable analogs thereof.

### Preparation of Compounds

The compounds used in the chemical reactions described herein are made according to organic synthesis techniques known to those skilled in this art, and may be made from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Enamine (Kyiv, UA), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Alternatively, specific and analogous reactants can be identified through the indices of known chemicals and reactions prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compound described herein is P. H. Stahl & C. G. Wermuth “Handbook of Pharmaceutical Salts”, Verlag Helvetica Chimica Acta, Zurich, 2002.

The compounds described herein are prepared using the general methods in the art of organic synthesis, for example, as described in the Examples section. Alternative synthetic methods are also used to generate the compounds described herein.

Some aspects of the compounds described herein include a recruiting ligand, which is configured to bind to a nucleic acid such that the target protein is targeted to the nucleic acid. In some embodiments the recruiting ligand binds to the nucleic acid. Some aspects relate to a compound comprising a targeting ligand, a recruiting ligand, and a linker; wherein the targeting ligand is attached to the recruiting ligand via the linker; wherein the recruiting ligand is configured to bind to a nucleic acid such that the target protein is targeted to the nucleic acid. The nucleic acid may include a DNA strand or sequence.

In some embodiments, the recruiting ligand can be a DNA binder covalently linked to a targeting ligand via a linker:

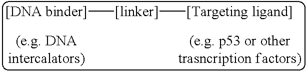

In some embodiments, the OmniTAC compound has the following structures:

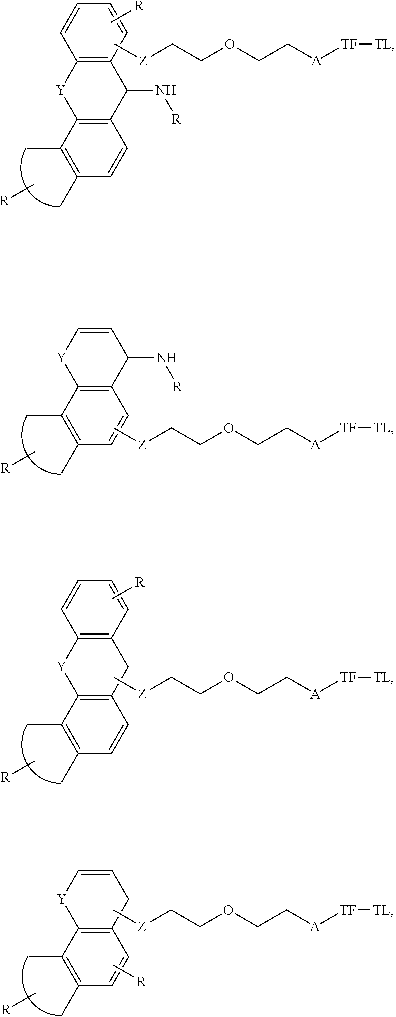

wherein:
A=attachment group (amide, triazole, ester, thioester, keto group, amine, carbamate (urethane), urea, carbon or ether in either orientation)
Y=NH, O, CO, CS, or S;
Z=NH, O, or CH₂;
R=any substituent group; and
TF-TL=transcription factor targeting ligand (e.g. p53 binders such as p53-TLa, p53-TLb and p53-TLc).

### Pharmaceutical Compositions

Disclosed herein, in some embodiments, are pharmaceutical compositions. The pharmaceutical composition may include a compound described herein. For example, the pharmaceutical composition may include an OmniTAC compound. In some embodiments, the pharmaceutical composition comprises a heterobifunctional compound. In some embodiments, the pharmaceutical composition is sterile. The pharmaceutical composition may include the compound and a pharmaceutically acceptable carrier.

In some embodiments, the pharmaceutically acceptable carrier comprises a water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution.

### Engineered Proteins

Disclosed herein, in some embodiments, are in vivo engineered proteins. Some embodiments include an in vivo engineered protein complex comprising a compound such as an OmniTAC compound directly bound to a target protein at one end of the compound and a modifier protein at another end of the compound.

Some embodiments include an in vivo engineered protein comprising: a target protein directly bound to a compound such as OmniTAC compound. In some embodiments, the compound comprises a targeting ligand. In some embodiments, the targeting ligand binds to a binding region on the target protein. In some embodiments, the binding region on the target protein comprises a particular domain or amino acid residue. In some embodiments, the target protein is directly bound to the targeting ligand by a non-covalent interaction between the target protein and the targeting ligand. In some embodiments, the binding between the target protein and the targeting ligand comprises a binding affinity described herein. In some embodiments, the compound described herein further comprises a recruiting ligand. In some embodiments, the recruiting ligand is attached to the targeting ligand via a linker such as a linker described herein. The target protein may include a structural change and/or modification, as compared to the same target protein not bound to the compound, such as a modification and/or structural change resulting from association with the modifier protein.

Some embodiments include an in vivo engineered protein comprising a modifier protein directly bound to a compound such as an OmniTAC compound. In some embodiments, the compound comprises a recruiting ligand. In some embodiments, the recruiting ligand binds to a binding region on the modifier protein. In some embodiments, the binding region on the modifier protein comprises a particular domain or amino acid residue. In some embodiments, the modifier protein is directly bound to the recruiting ligand by a non-covalent interaction between the modifier protein and the recruiting ligand. In some embodiments, the binding between the modifier protein and the recruiting ligand comprises a binding affinity described herein. In some embodiments, the compound described herein further comprises a targeting ligand. In some embodiments, the targeting ligand is attached to the recruiting ligand via a linker such as a linker described herein.

The modifier protein may be any modifier protein disclosed herein. The target protein may be any target protein disclosed herein. In some embodiments, the compound comprises a heterobifunctional compound. In some embodiments, the compound comprises an OmniTAC compound. In some embodiments, the compound comprises a targeting ligand, a recruiting ligand, and a linker. In some embodiments, a modified protein disclosed herein is formed in vivo upon administration of the compound or pharmaceutical composition to a subject. Some embodiments relate to a method of forming an in vivo engineered protein, comprising administering the compound or pharmaceutical composition to the subject.

### Methods of Using OmniTAC Compounds

Disclosed herein, in some embodiments, are methods of using a compound or pharmaceutical composition disclosed herein. For example, a method may include use of an OmniTAC compound. In some embodiments, a method includes use of a heterobifunctional compound. Some embodiments include a method for inducing a change in a target protein, comprising: contacting the target protein with a modifier protein via a modifier protein targeting chimeric compound (OmniTAC) such that the modifier protein induces a change to the target protein. The change may be or may include a modification described herein, and/or a structural change described herein. The change may include a change in activity of the target protein. The change may include a change in cellular location and/or cellular sublocalization of the target protein.

Some embodiments include a method for inducing a change in a VHL protein, comprising contacting the VHL protein with a modifier protein via a compound described herein, e.g. OmniTAC compound, such that the modifier protein induces a change to the VHL protein. Examples of ligands that bind VHL are included in FIG. 3A and FIG. 3B.

Some embodiments include a method for inducing a change in a P53 protein, comprising contacting the P53 protein with a modifier protein via a modifier protein targeting chimeric compound (OmniTAC) such that the modifier protein induces a change to the P53 protein. The change may include phosphorylation. Examples of ligands that bind P53 are included in FIG. 2A and FIG. 2B. Some embodiments include a method of activating a p53 mutant protein. The method may comprise contacting the p53 mutant protein with a compound disclosed herein, e.g., an OmniTAC compound.

The change in the target protein, e.g. p53, VHL, GBA, or any of the target proteins provided herein, may be or may include any change described herein. In some embodiments, the change includes phosphorylation. In some embodiments, the change includes dephosphorylation. In some embodiments, the change includes palmitoylation. In some embodiments, the change includes depalmitoylation. In some embodiments, the change includes methylation. In some embodiments, the change includes demethylation. In some embodiments, the change includes glycosylation. In some embodiments, the change includes deglycosylation. In some embodiments, the change includes ligation. In some embodiments, the change includes cleavage. In some embodiments, the change includes sulfonation. In some embodiments, the change includes desulfonation. In some embodiments, the change includes ubiquitylation. In some embodiments, the change includes deubiquitylation. In some embodiments, the change includes SUMOylation. In some embodiments, the change includes deSUMOylation. In some embodiments, the change includes fucosylation. In some embodiments, the change includes defucosylation. In some embodiments, the change includes sialylation. In some embodiments, the change includes desialylation. In some embodiments, the change includes tyrosylation. In some embodiments, the change includes detyrosylation. In some embodiments, the change includes acylation. In some embodiments, the change includes deacylation. In some embodiments, the change includes acetylation. In some embodiments, the change includes deacetylation. In some embodiments, the change includes decanoylation. In some embodiments, the change includes dedecanoylation. In some embodiments, the change includes a structural change. In some embodiments, the change includes binding or aggregation with another protein. In some embodiments, the change includes protein folding. In some embodiments, the change includes protein misfolding. In some embodiments, the change includes changes in cellular sublocalization. In some embodiments, the change includes nuclear localization. In some embodiments, the change includes mitochondrial localization.

The method may include measuring the change. The measurement may be made after administration of the compound to a cell or subject. The measurement may be made in relation to a control or baseline measurement.

The measurement may be made by any of a variety of methods. Measuring the change may include using a detection reagent that binds to a target protein and yields a detectable signal. After use of the detection reagent that binds to the target protein and yields the detectable signal, a readout may be obtained that is indicative of the presence, absence or amount of the change in the target protein. Measuring the change may include concentrating, filtering, or centrifuging a sample, e.g. comprising the target protein, the modifier protein, and/or the compound described herein.

Measuring the change may include using an assay method such as microscopy, spectrophotometry, mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof. The measurement may be obtained using mass spectrometry. Some examples of assay methods may include using mass spectrometry, a protein chip, or a reverse-phased protein microarray. A measurement may be generated using an immunoassay such as an enzyme-linked immunosorbent assay, western blot, dot blot, or immunohistochemistry. The measurement may be obtained using sequencing. A measurement may be obtained using flow cytometry. A measurement may be obtained using chromatography, for example high performance liquid chromatography.

The change may be measured or assessed using an enzyme activity assay. The change may be measured or assessed using histochemistry or immunohistochemistry. The change may be measured or assessed using microscopy. The change may be assessed using an assay such as an immunoassay, a colorimetric assay, a lateral flow assay, a fluorescence assay, a proteomics assay, or a cell-based assay.

The change may be measured using a reporter gene assay, e.g., a luciferase reporter gene assay. The change may be measured or assessed using luminescence or fluorescence. The change may be measured or assessed using microscopy. The change may be measured or assessed or normalized using cell viability.

Any compound described herein may be used in a method herein. For example, the compound comprises a targeting ligand, a recruiting ligand, and a linker. In some embodiments, the targeting ligand is configured to bind to a target protein described herein. In some embodiments, the recruiting ligand is configured to bind to a modifier protein described herein.

The method may be performed in a subject. The method may be performed in a cell or a sample of a subject. Examples of subjects include vertebrates, animals, mammals, dogs, cats, cattle, rodents, mice, rats, primates, monkeys, and humans. In some embodiments, the subject is a mammal. In some embodiments, the subject is a human.

In certain embodiments, the compounds described herein are used to treat a subject. The method may include administering a compound, e.g., a heterobifunctional compound described herein such as an OmniTAC compound. In some embodiments, a pharmaceutical composition described herein is administered. The subject may have a disease, and may be in need of treatment thereof. In some embodiments, the administration improves a symptom or parameter of the disease. In some embodiments, the disease is caused by a dysfunction and/or dysregulation of a target protein described herein, e.g., p53, VHL, GBA, or combination thereof. In some embodiments, the disease is cancer. In some embodiments, the disease is a genetic disease. In some embodiments, the disease is a neurological disease. The symptoms may be improved as assessed by a measurement made in relation to a baseline measurement.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity or ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. As used herein, “between” is a range inclusive of the ends of the range. For example, a number between x and y explicitly includes the numbers x and y, and any numbers that fall within x and y.

The section headings used herein are for organizational purposes only and are not to be construed as limiting the subject matter described.

As used in the specification and appended claims, unless specified to the contrary, the following terms have the meaning indicated below.

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 15% of that number. The term “about” a range refers to that range minus 15% of its lowest value and plus 15% of its greatest value.

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps. It is contemplated that any embodiment discussed in this specification can be implemented with respect to any compound, protein, and/or method of the present disclosure.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “protein” may include a polypeptide. A protein may include a eukaryotic protein, or a protein in a cell or subject.

As used herein, the terms “treatment” or “treating” are used in reference to a pharmaceutical or other intervention regimen for obtaining beneficial or desired results in the recipient. Beneficial or desired results include but are not limited to a therapeutic benefit or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₈ alkyl). In other embodiments, an alkyl comprises three to five carbon atoms (e.g., C₃-C₈ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₈ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₈ alkylene). In other embodiments, an alkylene comprises three to five carbon atoms (e.g., C₃-C₈ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) p-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a}(where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” or “heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) p-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

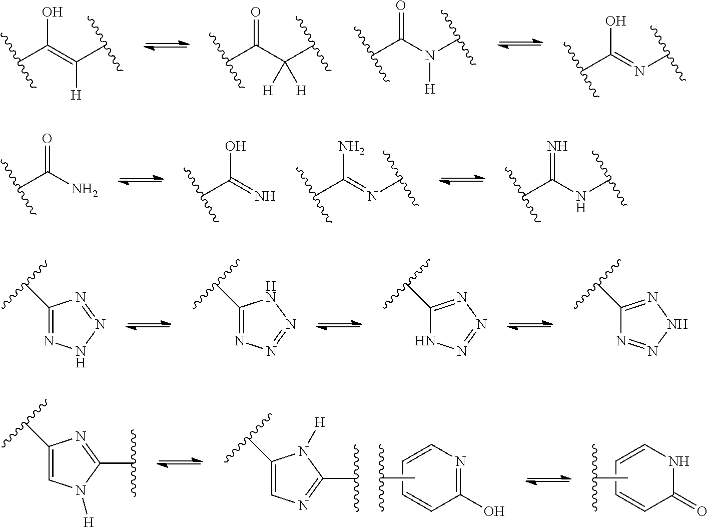

The compounds disclosed herein, in some embodiments, are used in different enriched isotopic forms, e.g., enriched in the content of ²H, ³H, ¹¹C, ¹³C or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Isotopic labeling can be performed using a variety of readily available reagents not limited to: isotopically-enriched carbon dioxide, dimethylformamide, cyanide salts, acetylenes, ammonium salts, other small organic and inorganic sources. Various methods are employed such as described in: Neumann, K. T. et al. Synthesis and Selective 2H-, 13C-, and 15N-Labeling of the Tau Protein Binder THK-523. J. Labelled Compd. Radiopharm. 2017, 60, 30-35; Baldwin, M. A.; Langley, G. J. Synthesis of [2-13C] Quinoline and [3-13C] Quinolone. J. Labelled Compd. Radiopharm. 1985, 22, 1233-1238: Wang, T. S. T. et al Preparation of 1-Methyl-4-[4-(7-chloro-4-quinolyl-[3-14C]-amino)benzoyl]piperazine. J. Labelled Compd. Radiopharm 1995, 36, 313-320; Saemian, N. et al Synthesis of 14C Analogue of 1,2-Diaryl-[2-14C]-pyrroles. J. Radioanal Nucl. Chem. 2007, 274, 631-634; Czeskis, B. A. et al. Synthesis of 14C-Labeled 4-Hydroxyindole as an Intermediate for the Preparation of (S)-2-[4-[2-[3-(Indol-2-[14C]-4-yloxy)-2-hydroxypropylamino]-2-methylpropyl]-phenoxy]pyridine-5-carboxamide (LY3688424[Indole-14C]) Glycolate. J. Labelled Compd. Radiopharm. 2102, 45, 1143-1152; Lath, B., et al. Synthesis of Two Potent Glucocorticoid Receptor Agonists Labeled with Carbon-14 and Stable Isotopes. J. Labelled Compd. Radiopharm. 2015, 58, 445-452; Cappon, J. J., et al Synthesis of L-Histidine Specifically Labelled with Stable Isotopes. Recl. Trav. Chim. Pays-Bas 1994, 113, 318-328; Guillame, M., et al. Unexpected Trifluoromethylated Pyrazoles From Ethyl 2-Diazo-4,4,4-trifluoroacetoacetate and 1-Diethylamino-prop-1-yne. J. Fluorine Chem. 1994, 69, 253-256; Hickey, M. J., et al. Syntheses of a Radiolabelled CXCR2 Antagonist AZD5069 and Its Major Human Metabolite, J. Labelled Compd. Radiopharm. 2016, 59, 432-438.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997); Gould, P. L. “Salt Selection for basic drugs” International Journal of Pharmaceutics, 33:201-217 (1986)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The publications, patents, and patent applications referenced herein are hereby incorporated by reference.

### EXAMPLES

The following examples are included for illustrative purposes only and are not intended to limit the scope of the invention.

### Example 1: Synthesis of {[9-ethyl-7-(4-methylthiophen-2-yl)-9H-carbazol-3-yl]methyl}(methyl)amine (Z5090393299)

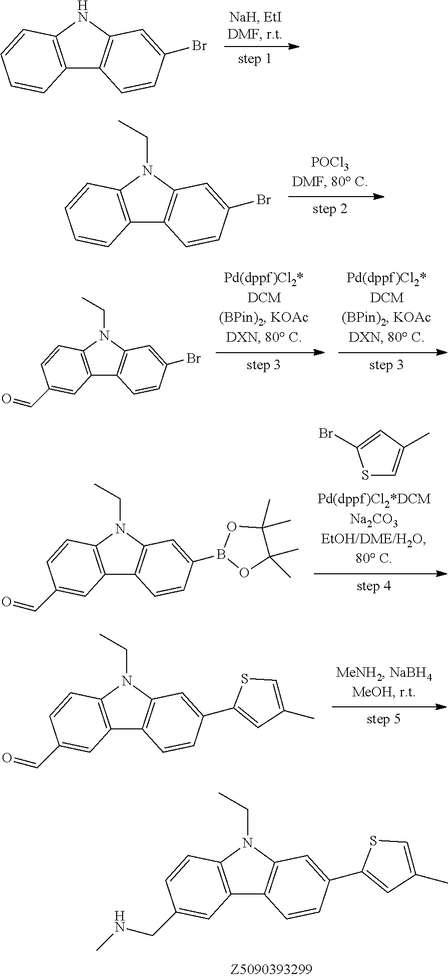

Step 1: The synthesis of 2-bromo-9-ethyl-9H-carbazole. To a suspension of sodium hydride (3.22 g, 134.09 mmol) in anhydrous DMF (150 ml) under inert atmosphere was added the solution of 2-bromo-9H-carbazole (18.0 g, 73.14 mmol) in DMF (50 ml). The resulting suspension was stirred at room temperature for 30 minutes. After that iodoethane (22.81 g, 146.28 mmol, 11.7 ml, 2.0 eq) was added dropwise and the resulting reaction mixture solution was left while stirring overnight. After 18 hours the reaction mixture was diluted with distilled water (400 ml) and extracted with ethyl acetate (3×200 ml). The organic layers were combined, washed with brine (3×100 ml), dried over anhydrous Na₂SO₄ and filtered. The filtrate collected was concentrated under reduced pressure to result in the desired 2-bromo-9-ethyl-9H-carbazole (20.0 g, 90.0% purity, 65.66 mmol, 89.8% yield) as beige solid. LCMS (2 min, 100% by UV 215 nm, RT 1.698, M=274.0).

Step 2: The synthesis of 7-bromo-9-ethyl-9H-carbazole-3-carbaldehyde. To a flask with dry DMF (20 ml) under argon at 0° C. phosphoryl trichloride (16.61 g, 108.33 mmol, 10.1 ml, 3.0 eq) was added dropwise and the resulting mixture was allowed to warm up to room temperature. After that the solution of 2-bromo-9-ethyl-9H-carbazole (9.9 g, 36.11 mmol) in dry DMF (20 ml) was added dropwise to the reaction mixture, which was then heated up to 80° C. and left while stirring for 42 hours. After that period the reaction mixture was cooled down to room temperature and poured onto ice (150 ml). The solution was adjusted to neutral pH with 10% aqueous solution of NaOH. The resulting aqueous solution was extracted with ethyl acetate (3×100 ml). The organic layers were combined, washed with brine (3×50 ml), dried over anhydrous Na₂SO₄ and filtered. The filtrate collected was concentrated under reduced pressure to result in 9 g of crude product that was subjected for flash chromatography purification (Interchim, 200 g SiO2, petroleum ether/ethyl acetate with ethyl acetate from 0˜95%, flow rate=60 ml/min, Rf=8.3 CV) to result in the desired 7-bromo-9-ethyl-9H-carbazole-3-carbaldehyde (4.5 g, 90.0% purity, 13.4 mmol, 37.1% yield). LCMS (2 min, 92.2% by UV 215 nm, RT 1.608, (M+)=302.0).

Step 3: The synthesis of 9-ethyl-7-(4,4,5,5-tetramethyl-1,3,2-dioxaborolan-2-yl)-9H-carbazole-3-carbaldehyde. To a solution of 7-bromo-9-ethyl-9H-carbazole-3-carbaldehyde (4.15 g, 13.73 mmol), 4,4,5,5-tetramethyl-2-(4,4,5,5-tetramethyl-1,3,2-dioxaborolan-2-yl)-1,3,2-dioxaborolane (5.23 g, 20.6 mmol) and potassium acetate (5.39 g, 54.93 mmol) in 1,4-dioxane (50 ml) under inert atmosphere (argon inlet) [1,1′-bis(diphenylphosphino)ferrocene]dichloropalladium (II) dichloromethane adduct (1.12 g, 1.37 mmol) was added. The resulting reaction mixture was heated up to 80° C. and left while stirring overnight. After 18 hours the rection mixture was concentrated under reduced pressure and the residue obtained was diluted with distilled water (100 ml) and extracted with ethyl acetate (3×50 ml). The organic layers were combined, washed with brine (50 ml), dried over anhydrous Na₂SO₄ and filtered. The filtrate collected was concentrated under reduced pressure and the crude obtained was subjected for flash chromatography purification (Interchim, 220 g SiO2, petroleum ether/ethyl acetate with ethyl acetate from 20˜40%, flow rate=60 ml/min, Rf=3.7 CV) to result in the desired 9-ethyl-7-(4,4,5,5-tetramethyl-1,3,2-dioxaborolan-2-yl)-9H-carbazole-3-carbaldehyde (2.1 g, 90.0% purity, 5.41 mmol, 39.4% yield) as pale yellow solid. LCMS (2 min, 100% by UV 215 nm, RT 1.476, (M+)=350.2).

Step 4: The synthesis of 9-ethyl-7-(4-methylthiophen-2-yl)-9H-carbazole-3-carbaldehyde. The mixture of 9-ethyl-7-(4,4,5,5-tetramethyl-1,3,2-dioxaborolan-2-yl)-9H-carbazole-3-carbaldehyde (700.0 mg, 2.0 mmol), 2-bromo-4-methylthiophene (532.15 mg, 3.01 mmol) and sodium carbonate (254.83 mg, 2.4 mmol) in solvent system EtOH/DME/H2O (40/40/20 ml) was flushed with argon three times. After that [1,1′-bis(diphenylphosphino)ferrocene]dichloropalladium (II) dichloromethane adduct (163.62 mg, 200.36 gmol) was added to the reaction mixture under inert atmosphere. The resulting solution was heated up to 100° C. and left while stirring overnight. After 18 hours the mixture was concentrated under reduced pressure and the residue obtained was diluted with distilled water (100 ml) and extracted with ethyl acetate (3×50 ml). The organic layers were combined, washed with brine (50 ml), dried over anhydrous Na₂SO₄ and filtered. The filtrate collected was concentrated under reduced pressure. The crude product obtained was subjected for flash chromatography purification (Interchim; 40 g SiO2, petroleum ether/ethyl acetate with ethyl acetate from 0˜95%, flow rate=40 ml/min, Rt=18 min) to result in the desired 9-ethyl-7-(4-methylthiophen-2-yl)-9H-carbazole-3-carbaldehyde (120.0 mg, 90.0% purity, 338.11 gmol, 16.9% yield): LCMS (2 min, 85.7% by UV 215 nm, RT 1.628, (M+)=320.2).

Step 5: The synthesis of {[9-ethyl-7-(4-methylthiophen-2-yl)-9H-carbazol-3-yl]methyl}(methyl)amine (Z5090393299). The solution of 9-ethyl-7-(4-methylthiophen-2-yl)-9H-carbazole-3-carbaldehyde (60.0 mg, 187.84 gmol) in methanamine (192.43 mg, 6.2 mmol, 1.23 ml, 33.0 eq) was stirred at room temperature for 2 hours. After that period the mixture was evaporated in vacuo to dryness, and the residue obtained was dissolved in absolute methanol (2 ml), followed by portionwise addition of sodium borohydride (35.52 mg, 938.78 gmol) at 10° C. The reaction mixture was then allowed to warm up to room temperature and left while stirring overnight. After 18 hours the mixture was concentrated under reduced pressure and the residue obtained was diluted with 20% aqueous solution of K2CO3 (25 ml) and extracted with ethyl acetate (3×10 ml). The organic layers were combined, dried over anhydrous Na₂SO₄ and filtered. The filtrate collected was concentrated under reduced pressure and and the crude obtained was subjected for prep HPLC purification (30-70% 0.5-6.5 min water-acetonitrile; flow 30 ml/min; column SunFire 100×19 mm, 5 um) to result in the desired [9-ethyl-7-(4-methylthiophen-2-yl)-9H-carbazol-3-yl]methyl(methyl)amine (19.6 mg, 95.0% purity, 55.67 gmol, 29.6% yield) with the following spectra data: LCMS (6 min, 95.87% by UV 215 nm, RT 3.019, (M+)=304).

### Example 2: Synthesis of {[9-ethyl-7-(thiophen-2-yl)-9H-carbazol-3-yl]methyl}(methyl)amine (Z5094964725)

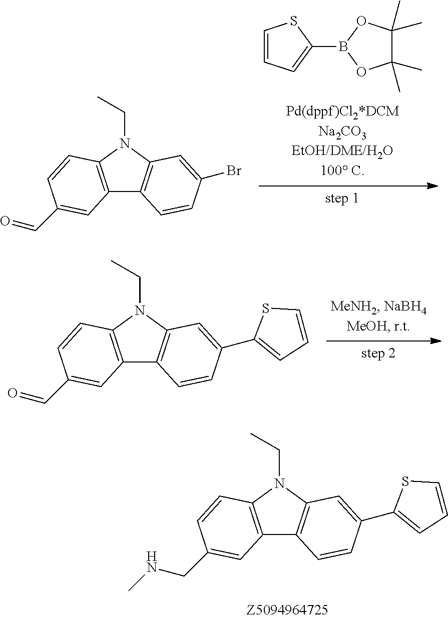 [SMILES: CC1(C)OB(c2cccs2)OC1(C)C ; CCn1c2ccc(C=O)cc2c2ccc(Br)cc21 ; CCn1c2ccc(C=O)cc2c2ccc(-c3cccs3)cc21 ; CCn1c2ccc(CNC)cc2c2ccc(-c3cccs3)cc21]

Step 1: The synthesis of 9-ethyl-7-(thiophen-2-yl)-9H-carbazole-3-carbaldehyde. The solution of 7-bromo-9-ethyl-9H-carbazole-3-carbaldehyde* (250.0 mg, 827.36 μmol), 4,4,5,5-tetramethyl-2-(thiophen-2-yl)-1,3,2-dioxaborolane (191.46 mg, 911.28 gmol) and sodium carbonate (105.37 mg, 994.13 gmol) in the mixture of EtOH, DME and H2O (20/20/10 ml) was flushed with argon three times. After that under inert atmosphere (argon inlet) [1,1′-bis(diphenylphosphino)ferrocene]dichloropalladium (II) dichloromethane adduct (67.65 mg, 82.84 gmol) was added to the reaction mixture, which was then heated up to 100° C. and left while stirring overnight. After 18 hours the mixture was concentrated under reduced pressure and the residue obtained was diluted with distilled water (100 ml) and extracted with ethyl acetate (3×50 ml). The organic layers were combined, washed with brine (50 ml), dried over anhydrous Na2SO4 and filtered. The filtrate collected was concentrated under reduced pressure and the crude product obtained was subjected for prep HPLC (70-100% 0.5-6.5 min water/acetonitrile; flow 30 ml/min; column SunFire C18 100×19 mm, 5 um) to result in the desired 9-ethyl-7-(thiophen-2-yl)-9H-carbazole-3-carbaldehyde (211.9 mg, 98.0% purity, 679.98 gmol, 82.1% yield): LCMS (2 min, 100% by UV 215 nm, RT 1.511, (M+)=306.0).

Step 2: The synthesis of {[9-ethyl-7-(thiophen-2-yl)-9H-carbazol-3-yl]methyl}(methyl)amine (Z5094964725). The solution of the starting 9-ethyl-7-(thiophen-2-yl)-9H-carbazole-3-carbaldehyde (30.02 mg, 98.3 gmol) in methanamine (3.05 mg, 98.3 gmol) was stirred at room temperature for 2 hours. After that period the mixture was evaporated in vacuo to dryness, and the residue obtained was dissolved in absolute MeOH (2 ml) and sodium borohydride (18.59 mg, 491.5 gmol) was added portionwise at 10° C. After the addition was completed the reaction mixture was allowed to warm up to room temperature and left while stirring overnight. After 15 hours the mixture was concentrated under reduced pressure. The residue obtained was diluted with 20% aqueous solution of K2CO3 (25 ml) and extracted with ethyl acetate (3×10 ml). The organic layers were combined, dried over anhydrous Na2SO4 and filtered. The filtrate collected was concentrated under reduced pressure and the crude product obtained was subjected for prep HPLC purification (25-50% 0.5-6.5 min water/acetonitrile+TFA; flow 30 ml/min; column SunFire C18 100×19 mm, 5 um) to result in the desired [9-ethyl-7-(thiophen-2-yl)-9H-carbazol-3-yl]methyl(methyl)amine (5.1 mg, 95.0% purity, 15.12 gmol, 15.4% yield) as TFA salt with the following spectra data: LCMS (6 min, 100% by UV 215 nm, RT 3.103, (M+)=290.2).

### Example 3: Synthesis of 6-(16-azido-5,8,11,14-tetraoxa-2-azahexadecan-1-yl)-9-ethyl-2-(thiophen-2-yl)-9H-carbazole (Z6644467484)

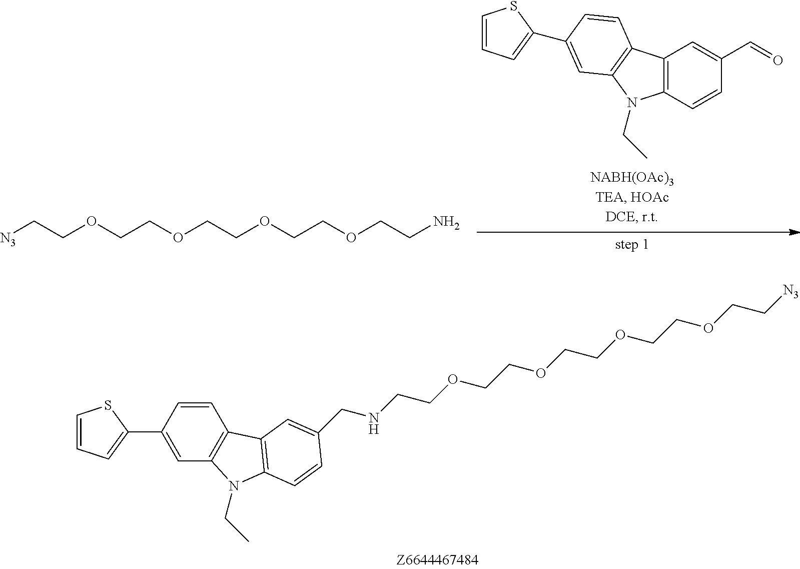 [SMILES: CCn1c2ccc(C=O)cc2c2ccc(-c3cccs3)cc21 ; [N-]=[N+]=NCCOCCOCCOCCOCCN ; CCn1c2ccc(CNCCOCCOCCOCCOCCN=[N+]=[N-])cc2c2ccc(-c3cccs3)cc21]

Step 1: The starting 9-ethyl-7-(thiophen-2-yl)-9H-carbazole-3-carbaldehyde (1.0 g, 3.27 mmol) and 14-azido-3,6,9,12-tetraoxatetradecan-1-amine (945.05 mg, 3.6 mmol) were mixed in anhydrous dichloroethane (50 ml), after that acetic acid (19.67 mg, 327.53 gmol) was added to the resulting solution, followed by the addition of triethylamine (662.86 mg, 6.55 mmol) and sodium bis(acetyloxy)boranuidyl acetate (2.78 g, 13.1 mmol). The resulting reaction mixture suspension was stirred at room temperature for 48 hours. After that the mixture was concentrated under reduced pressure and the residue obtained was diluted with 20% aqueous solution of K2CO3 (50 ml) and extracted with DCM (3×25 ml). The organic layers were combined, dried over anhydrous Na2SO4 and filtered. The filtrate collected was concentrated under reduced pressure and the crude obtained was subjected for flash chromatography purification (Interchim; 40 g SiO2, MtBE/methanol with methanol from 0˜95%, flow rate=40 ml/min, Rf=7-12 CV) to result in the desired 6-(16-azido-5,8,11,14-tetraoxa-2-azahexadecan-1-yl)-9-ethyl-2-(thiophen-2-yl)-9H-carbazole (1.1 g, 95.0% purity, 1.89 mmol, 57.8% yield). LCMS (2 min, 92.9% by UV 215 nm, RT 1.386, (M+)=552.2).

### Example 4: Synthesis of 1-4-[2-(2,4-dichlorophenyl)-2-[(naphthalen-2-yl)methoxy]ethyl]piperazin-1-yl-4-(1-1-[9-ethyl-7-(thiophen-2-yl)-9H-carbazol-3-yl]-5,8,11,14-tetraoxa-2-azahexadecan-16-yl-1H-1,2,3-triazol-4-yl)butan-1-one (Z6737047626)

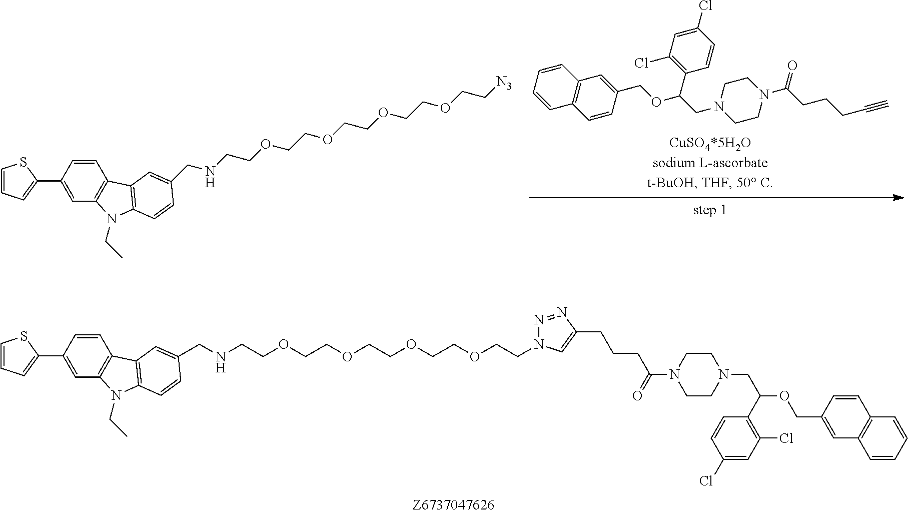

Step 1: To a solution of 1-4-[2-(2,4-dichlorophenyl)-2-[(naphthalen-2-yl)methoxy]ethyl]piperazin-1-ylhex-5-yn-1-one (27.7 mg, 54.37 gmol) in the mixture of t-BuOH (2 ml) and distilled H2O (1 ml) at 0° C. was added sodium (2R)-2-[(1S)-1,2-dihydroxyethyl]-4-hydroxy-5-oxo-2,5-dihydrofuran-3-olate (10.77 mg, 54.37 gmol), followed by the addition of copper (II) sulfate pentahydrate (6.79 mg, 27.19 gmol). After 5 min of stirring the solution of 6-(16-azido-5,8,11,14-tetraoxa-2-azahexadecan-1-yl)-9-ethyl-2-(thiophen-2-yl)-9H-carbazole (30.0 mg, 54.38 gmol) in THF (1 ml) was added to the reaction mixture, which was then heated up to 50° C. and left while stirring overnight. After the reaction was completed (monitored by LCMS), the mixture was concentrated under reduced pressure and the residue was diluted with DMSO (1 ml), filtered through celite and the filtrate collected was subjected for prep HPLC without any further work-up (60-100% 0-8 min water/methanol, flow 30 ml/min, column: Chromatorex 18 SMB100-ST, 100×19 mm, 5 um) to give the desired 1-4-[2-(2,4-dichlorophenyl)-2-[(naphthalen-2-yl)methoxy]ethyl]piperazin-1-yl-4-(1-1-[9-ethyl-7-(thiophen-2-yl)-9H-carbazol-3-yl]-5,8,11,14-tetraoxa-2-azahexadecan-16-yl-1H-1,2,3-triazol-4-yl)butan-1-one (26 mg, 24 gmol, 45% yield): LCMS (6 min, 100% by UV 215 nm, RT 3.928, (M+)=1060.2).

### Example 5: Synthesis of 1-[7-(3,4-dimethoxyphenyl)-9-{[(3S)-1-[2-(1-{1-[9-ethyl-7-(thiophen-2-yl)-9H-carbazol-3-yl]-5,8,11,14-tetraoxa-2-azahexadecan-16-yl}-1H-1,2,3-triazol-4-yl)ethyl]piperidin-3-yl]methoxy}-2,3,4,5-tetrahydro-1,4-benzoxazepin-4-yl]propan-1-one (Z6737047615)

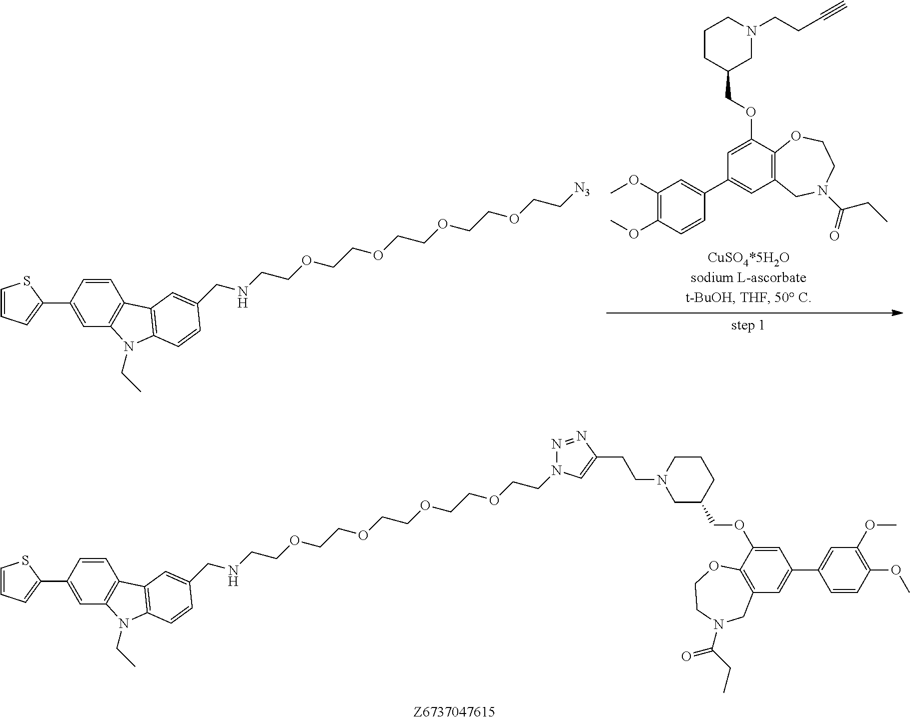

Step 1: Z6737047615 compound was obtained according to the procedure described for the synthesis of Z6737047626. As a result, after final prep HPLC purification (50-90% 0-8 min water/methanol, flow 30 ml/min, column: Chromatorex 18 SMB100-ST 100×19 mm, 5 um) the desired 1-[7-(3,4-dimethoxyphenyl)-9-[(3S)-1-[2-(1-1-[9-ethyl-7-(thiophen-2-yl)-9H-carbazol-3-yl]-5,8,11,14-tetraoxa-2-azahexadecan-16-yl-1H-1,2,3-triazol-4-yl)ethyl]piperidin-3-yl]methoxy-2,3,4,5-tetrahydro-1,4-benzoxazepin-4-yl]propan-1-one (12.7 mg, 97.0% purity, 11.64 gmol, 21.4% yield) was obtained. LCMS (6 min, 96.63% by UV 215 nm, RT 2.901, (M+)=1059.6).

### Example 6: Synthesis of 7-(3,5-dimethoxyphenyl)-N-[(3S)-1-[2-(1-{1-[9-ethyl-7-(thiophen-2-yl)-9H-carbazol-3-yl]-5,8,11,14-tetraoxa-2-azahexadecan-16-yl}-1H-1,2,3-triazol-4-yl)ethyl]piperidin-3-yl]-4-propanoyl-2,3,4,5-tetrahydro-1,4-benzoxazepine-9-carboxamide (Z6737047671)

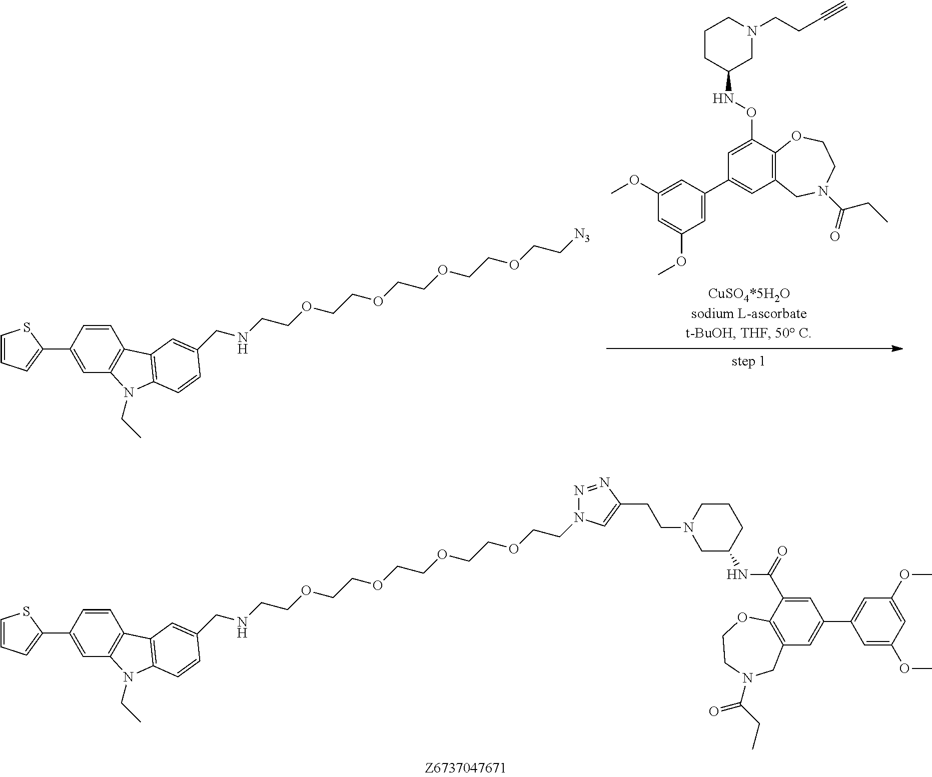 [SMILES: CCn1c2ccc(CNCCOCCOCCOCCOCCN=[N+]=[N-])cc2c2ccc(-c3cccs3)cc21 ; C#CCCN1CCC[C@H](NOc2cc(-c3cc(OC)cc(OC)c3)cc3c2OCCN(C(=O)CC)C3)C1 ; CCC(=O)N1CCOc2c(cc(-c3cc(OC)cc(OC)c3)cc2C(=O)N[C@H]2CCCN(CCc3cn(CCOCCOCCOCCOCCNCc4ccc5c(c4)c4ccc(-c6cccs6)cc4n5CC)nn3)C2)C1]

Step 1: Z6737047671 compound was obtained according to the procedure described for the synthesis of Z6737047626. As a result, after final prep HPLC purification (50-100% 0-6 min water/methanol, flow 30 ml/min, column: Chromatorex 18 SMB100-ST 100×19 mm, 5 um) the desired 7-(3,5-dimethoxyphenyl)-N-[(3S)-1-[2-(1-1-[9-ethyl-7-(thiophen-2-yl)-9H-carbazol-3-yl]-5,8,11,14-tetraoxa-2-azahexadecan-16-yl-1H-1,2,3-triazol-4-yl)ethyl]piperidin-3-yl]-4-propanoyl-2,3,4,5-tetrahydro-1,4-benzoxazepine-9-carboxamide (16.6 mg, 99.0% purity, 15.34 gmol, 28.2% yield) was obtained. LCMS (6 min, 98.66% by UV 215 nm, RT 2.994, (M+)=1072.4).

### Example 7: Synthesis of 6-(16-4-[(4-2-[5-(3,5-dimethyl-1,2-oxazol-4-yl)-1-[2-(morpholin-4-yl)ethyl]-1H-1,3-benzodiazol-2-yl]ethylphenoxy)methyl]-1H-1,2,3-triazol-1-yl-5,8,11,14-tetraoxa-2-azahexadecan-1-yl)-9-ethyl-2-(thiophen-2-yl)-9H-carbazole (Z6737047683)

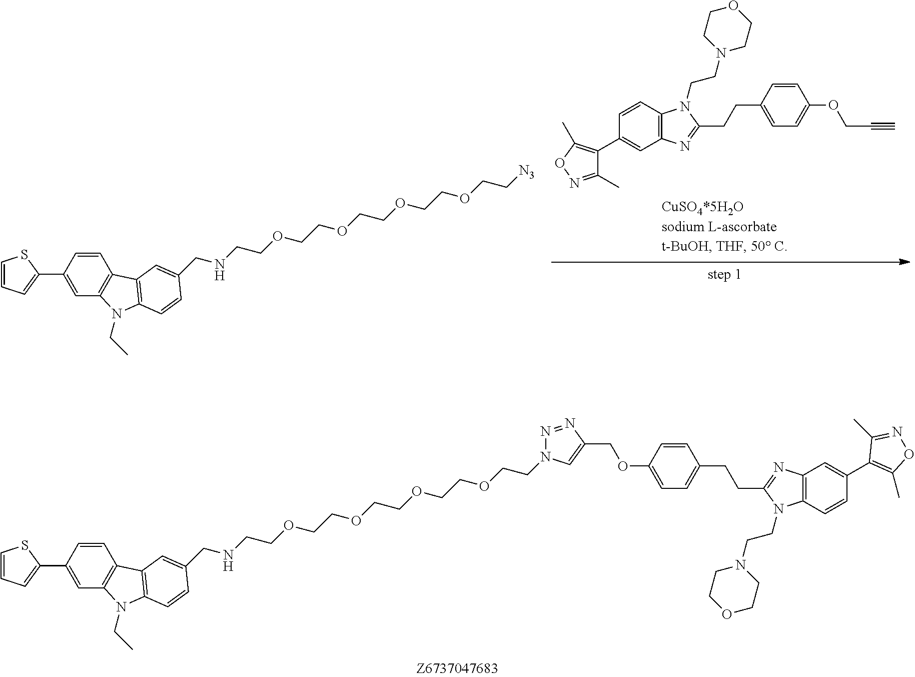 [SMILES: CCn1c2ccc(CNCCOCCOCCOCCOCCN=[N+]=[N-])cc2c2ccc(-c3cccs3)cc21 ; C#CCOc1ccc(CCc2nc3cc(-c4c(C)noc4C)ccc3n2CCN2CCOCC2)cc1 ; CCn1c2ccc(CNCCOCCOCCOCCOCCn3cc(COc4ccc(CCc5nc6cc(-c7c(C)noc7C)ccc6n5CCN5CCOCC5)cc4)nn3)cc2c2ccc(-c3cccs3)cc21]

Step 1: Z6737047683 compound was obtained according to the procedure described for the synthesis of Z6737047626. As a result, after final prep HPLC purification (60-100% 0-7 min water/methanol, flow 30 ml/min, column: XBridge C18 OBD 100×19 mm, 5 um) the desired 6-(16-4-[(4-2-[5-(3,5-dimethyl-1,2-oxazol-4-yl)-1-[2-(morpholin-4-yl)ethyl]-1H-1,3-benzodiazol-2-yl]ethylphenoxy)methyl]-1H-1,2,3-triazol-1-yl-5,8,11,14-tetraoxa-2-azahexadecan-1-yl)-9-ethyl-2-(thiophen-2-yl)-9H-carbazole (9.9 mg, 9.55 gmol, 17.6% yield) was obtained. LCMS (6 min, 100% by UV 215 nm, RT 3.514, (M+)=1036.2).

### Example 8: Synthesis of 3-[(4-{4-[(1-{1-[9-ethyl-7-(thiophen-2-yl)-9H-carbazol-3-yl]-5,8,11,14-tetraoxa-2-azahexadecan-16-yl}-1H-1,2,3-triazol-4-yl)methoxy]benzenesulfonyl}-1,4-diazepan-1-yl)sulfonyl]aniline (Z6737047657)

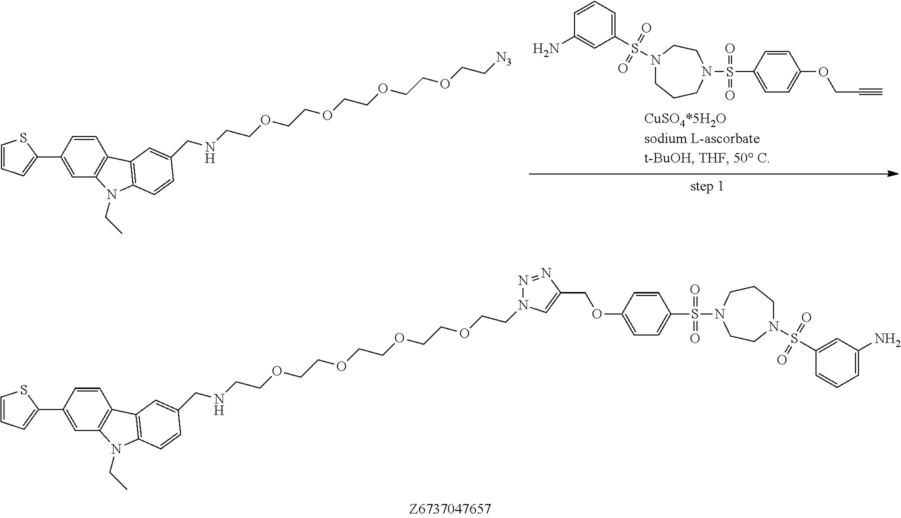 [SMILES: CCn1c2ccc(CNCCOCCOCCOCCOCCN=[N+]=[N-])cc2c2ccc(-c3cccs3)cc21 ; C#CCOc1ccc(S(=O)(=O)N2CCCN(S(=O)(=O)c3cccc(N)c3)CC2)cc1 ; CCn1c2ccc(CNCCOCCOCCOCCOCCn3cc(COc4ccc(S(=O)(=O)N5CCCN(S(=O)(=O)c6cccc(N)c6)CC5)cc4)nn3)cc2c2ccc(-c3cccs3)cc21]

Step 1: Z6737047657 compound was obtained according to the procedure described for the synthesis of Z6737047626. As a result, after final prep HPLC purification (50-100% 0-6 min water/methanol, flow 30 ml/min, column: Chromatorex 18 SMB100-ST 100×19 mm, 5 um) the desired 3-[(4-4-[(1-1-[9-ethyl-7-(thiophen-2-yl)-9H-carbazol-3-yl]-5,8,11,14-tetraoxa-2-azahexadecan-16-yl-1H-1,2,3-triazol-4-yl)methoxy]benzenesulfonyl-1,4-diazepan-1-yl)sulfonyl]aniline (14.3 mg, 97.0% purity, 13.85 gmol, 25.5% yield) was obtained. LCMS (6 min, 97.42% by UV 215 nm, RT 3.387, (M+)=1003.2).

### Example 9: Synthesis of 2-chloro-10-{4-[(1-{1-[9-ethyl-7-(thiophen-2-yl)-9H-carbazol-3-yl]-5,8,11,14-tetraoxa-2-azahexadecan-16-yl}-1H-1,2,3-triazol-4-yl)methoxy]benzoyl}-10H-phenothiazine (Z6737047622)

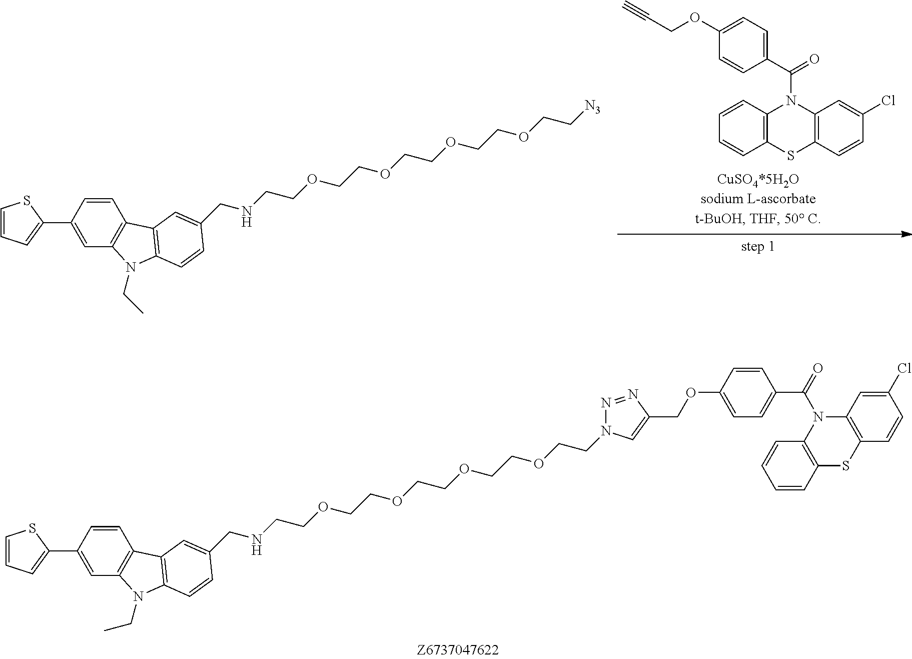 [SMILES: CCn1c2ccc(CNCCOCCOCCOCCOCCN=[N+]=[N-])cc2c2ccc(-c3cccs3)cc21 ; C#CCOc1ccc(C(=O)N2c3ccccc3Sc3ccc(Cl)cc32)cc1 ; CCn1c2ccc(CNCCOCCOCCOCCOCCn3cc(COc4ccc(C(=O)N5c6ccccc6Sc6ccc(Cl)cc65)cc4)nn3)cc2c2ccc(-c3cccs3)cc21]

Step 1: Z6737047622 compound was obtained according to the procedure described for the synthesis of Z6737047626. As a result, after final prep HPLC purification (70-100% 0-8 min water/methanol, flow 30 ml/min, column: Chromatorex C18 SMB100-ST 100×19 mm, 5 um) the desired 2-chloro-10-4-[(1-1-[9-ethyl-7-(thiophen-2-yl)-9H-carbazol-3-yl]-5,8,11,14-tetraoxa-2-azahexadecan-16-yl-1H-1,2,3-triazol-4-yl)methoxy]benzoyl-10H-phenothiazine (13.4 mg, 14.2 gmol, 26.1% yield) was obtained. LCMS (6 min, 100% by UV 215 nm, RT 4,836, (M+)=945.8).

### Example 10: Synthesis of 2-[bis(2-methylpropyl)amino]-1-{4-[2-(2,4-dichlorophenyl)-2-({4-[(1-{1-[9-ethyl-7-(thiophen-2-yl)-9H-carbazol-3-yl]-5,8,11,14-tetraoxa-2-azahexadecan-16-yl}-1H-1,2,3-triazol-4-yl)methoxy]phenyl}methoxy)ethyl]piperazin-1-yl}ethan-1-one (Z6737047600)

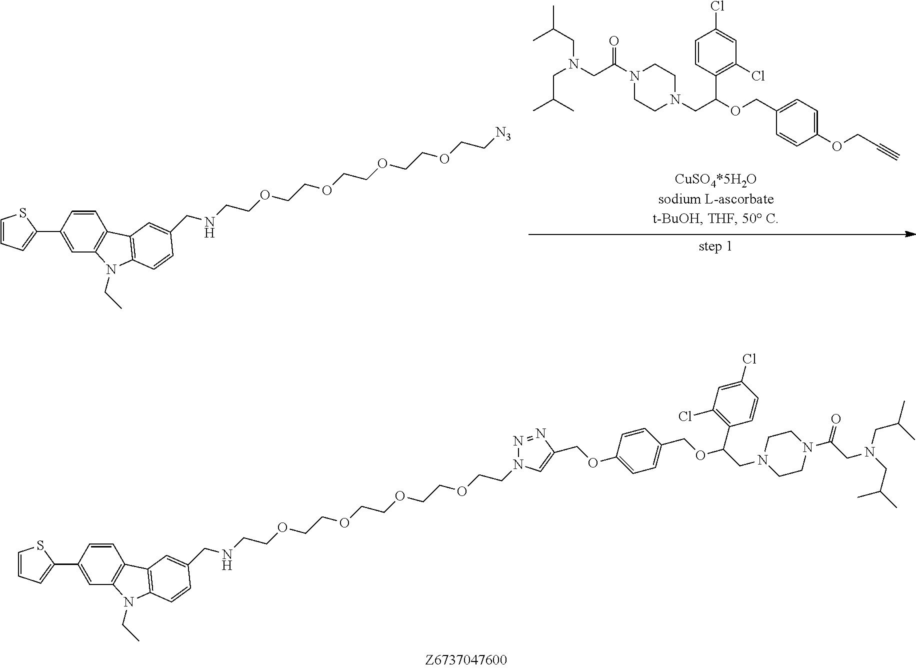 [SMILES: CCn1c2ccc(CNCCOCCOCCOCCOCCN=[N+]=[N-])cc2c2ccc(-c3cccs3)cc21 ; C#CCOc1ccc(COC(CN2CCN(C(=O)CN(CC(C)C)CC(C)C)CC2)c2ccc(Cl)cc2Cl)cc1 ; CCn1c2ccc(CNCCOCCOCCOCCOCCn3cc(COc4ccc(COC(CN5CCN(C(=O)CN(CC(C)C)CC(C)C)CC5)c5ccc(Cl)cc5Cl)cc4)nn3)cc2c2ccc(-c3cccs3)cc21]

Step 1: Z6737047600 compound was obtained according to the procedure described for the synthesis of Z6737047626. As a result, after final prep HPLC purification (60-100% 0-8 min water/methanol, flow 30 ml/min, column: XBridge C18 OBD 100×19 mm, 5 um) the desired 2-[bis(2-methylpropyl)amino]-1-4-[2-(2,4-dichlorophenyl)-2-(4-[(1-1-[9-ethyl-7-(thiophen-2-yl)-9H-carbazol-3-yl]-5,8,11,14-tetraoxa-2-azahexadecan-16-yl-1H-1,2,3-triazol-4-yl)methoxy]phenylmethoxy)ethyl]piperazin-1-ylethan-1-one (16.2 mg, 14.21 gmol, 43.1% yield) was obtained. LCMS (Z118991$1, 6 min, 100% by UV 215 nm, RT 3.309, (M+)=1141.6).

### Example 11: Synthesis of (2S,4R)-1-[(2S)-2-[4-(1-{15-[(2-{[(4-tert-butylcyclohexyl)(methyl)amino]methyl}quinazolin-4-yl)amino]-12-methyl-3,6,9-trioxa-12-azapentadecan-1-yl}-1H-1,2,3-triazol-4-yl)butanamido]-3,3-dimethylbutanoyl]-4-hydroxy-N-{[4-(4-methyl-1,3-thiazol-5-yl)phenyl]methyl}pyrrolidine-2-carboxamide (Z5454478682)

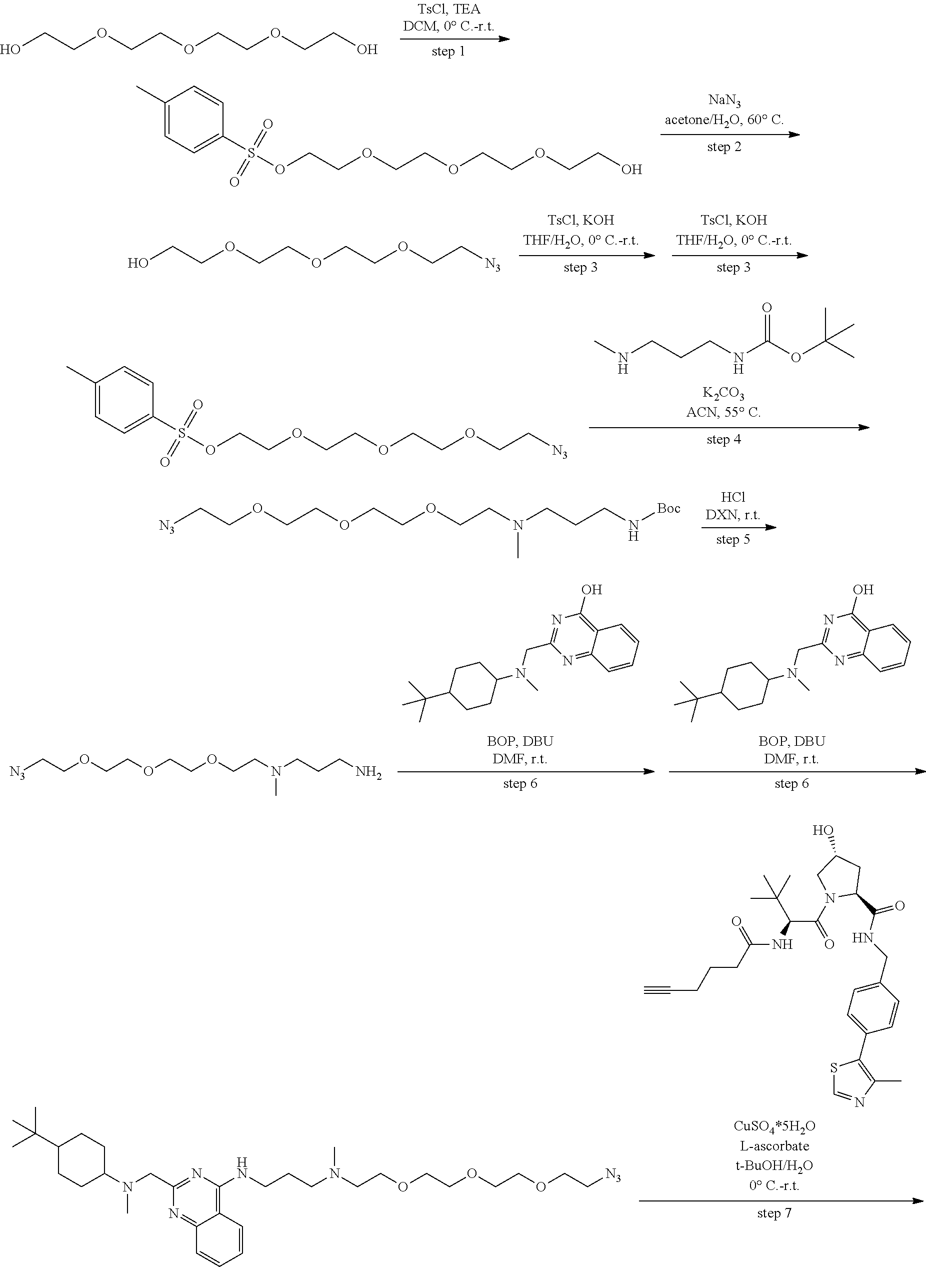

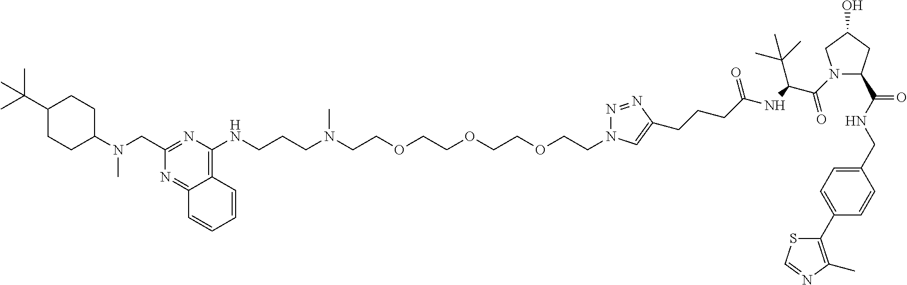 [SMILES: Cc1ncsc1-c1ccc(CNC(=O)[C@@H]2C[C@@H](O)CN2C(=O)[C@@H](NC(=O)CCCc2cn(CCOCCOCCOCCN(C)CCCNc3nc(CN(C)C4CCC(C(C)(C)C)CC4)nc4ccccc34)nn2)C(C)(C)C)cc1]

Step 1: The synthesis of 2-[2-(2-{2-[(4-methylbenzenesulfonyl)oxy]ethoxy}ethoxy)ethoxy]ethan-1-ol. A solution of both 2-2-[2-(2-hydroxyethoxy)ethoxy]ethoxyethan-1-ol (150.0 g, 772.3 mmol) and triethylamine (35.17 g, 347.54 mmol, 48.44 ml, 1.5 eq) in DCM (3000 ml) was cooled with an ice/water bath. After that the solution of 4-methylbenzene-1-sulfonyl chloride (44.17 g, 231.69 mmol) in DCM (1000 ml) was added dropwise to the reaction mixture. After the addition was completed, the resulting mixture was allowed to warm up to room temperature and left while stirring overnight. After 15 hours the reaction mixture was washed with water (2×500 ml). The organic layer was separated, dried over anhydrous Na₂SO₄ and filtered. The filtrate collected was concentrated under reduced pressure and the crude residue obtained was subjected for FC purification (Interchim; 800 g SiO₂, MtBE/methanol with methanol from 0˜95%, flow rate=150 ml/min, R_{f}=3.7 CV) to afford the desired 2-[2-(2-2-[(4-methylbenzenesulfonyl)oxy]ethoxyethoxy)ethoxy]ethan-1-ol (36.0 g, 95.0% purity, 98.16 mmol, 42.4% yield). LCMS (2 min, 100% by UV 215 nm, RT 1.049, (M+)=349.0). The material was used as such in further experiments.

Step 2: The synthesis of 2-{2-[2-(2-azidoethoxy)ethoxy]ethoxy}ethan-1-ol. The starting 2-[2-(2-2-[(4-methylbenzenesulfonyl)oxy]ethoxyethoxy)ethoxy]ethan-1-ol (36.0 g, 103.33 mmol) was dissolved in acetone (500 ml), after that the solution of sodium azide (33.59 g, 516.63 mmol) in water (100 ml) was added to the reaction mixture, which was then heated up to 60° C. and left while stirring overnight. After 15 hours the mixture was concentrated under reduced pressure and the residue obtained was diluted with water and extracted with dichloromethane (3×200 ml). The organic layers were combined, dried over anhydrous Na2SO4 and filtered. The filtrate collected was concentrated under reduced pressure to afford the desired 2-2-[2-(2-azidoethoxy)ethoxy]ethoxyethan-1-ol (25.0 g, 90.0% purity, 102.63 mmol, 99.3% yield). LCMS (2 min, 100% by UV 215 nm, RT 0.705, (M+)=220.2). The product obtained was used in further experiments without any additional purification.

Step 3: The synthesis of 1-[(2-{2-[2-(2-azidoethoxy)ethoxy]ethoxy}ethoxy)sulfonyl]-4-methylbenzene. A solution of both 2-2-[2-(2-azidoethoxy)ethoxy]ethoxyethan-1-ol (15.0 g, 68.42 mmol) and 4-methylbenzene-1-sulfonyl chloride (19.57 g, 102.63 mmol) in THF (250 ml) was cooled with an ice/water bath. After that the solution of potassium hydroxide (11.52 g, 205.25 mmol) in water (25 ml) was added dropwise to the reaction mixture. After the addition was completed the reaction mixture was allowed to warm up to room temperature and left while stirring overnight. After 15 hours the reaction mixture was concentrated under reduced pressure and the residue obtained was diluted with DCM (250 ml) and washed with water (2×100 ml). The organic layer was separated, dried over anhydrous Na2SO4 and filtered. The filtrate collected was concentrated under reduced pressure to afford the desired 1-[(2-2-[2-(2-azidoethoxy)ethoxy]ethoxyethoxy)sulfonyl]-4-methylbenzene (20.0 g, 90.0% purity, 48.2 mmol, 70.5% yield). LCMS (2 min, 100% by UV 215 nm, RT 1.204, ((M−N2)+)=346.0). The product obtained was used in further experiments without any additional purification.

Step 4: The synthesis of tert-butyl N-(1-azido-12-methyl-3,6,9-trioxa-12-azapentadecan-15-yl)carbamate. The starting 1-[(2-2-[2-(2-azidoethoxy)ethoxy]ethoxyethoxy)sulfonyl]-4-methylbenzene (6.0 g, 16.07 mmol) and tert-butyl N-[3-(methylamino)propyl]carbamate (2.75 g, 14.61 mmol) were mixed together in dry acetonitrile (50 ml), after that potassium carbonate (6.06 g, 43.82 mmol) was added to the resulting solution. The reaction mixture was then left while vigorous stirring overnight at 55° C. After 15 hours the mixture was concentrated under reduced pressure and the residue obtained was diluted with ethyl acetate (50 ml) and washed with water (2×20 ml). The organic layer was separated, dried over anhydrous sodium sulfate and filtered. The filtrate collected was concentrated under reduced pressure to afford the crude product, which was subjected for flash chromatography purification (Interchim; 120 g SiO2, MtBE/methanol with methanol from 0˜95%, flow rate=60 ml/min, Rf=4.6 CV) to result in the desired tert-butyl N-(1-azido-12-methyl-3,6,9-trioxa-12-azapentadecan-15-yl)carbamate (2.4 g, 95.0% purity, 5.85 mmol, 40.1% yield) with the following spectra data: LCMS (2 min, 100% by UV 215 nm, RT 0.612, (M+)=390). The product obtained was used in further experiments without additional purification.

Step 5: The synthesis of 1-azido-12-methyl-3,6,9-trioxa-12-azapentadecan-15-amine. The starting tert-butyl N-(1-azido-12-methyl-3,6,9-trioxa-12-azapentadecan-15-yl)carbamate (2.4 g, 6.16 mmol) was dissolved in dry dioxane (20 ml) and 2.2M HCl in dioxane (20 ml) was added to the resulting solution. The reaction mixture was then left while stirring overnight at room temperature. After 12 hours the reaction mixture was concentrated under reduced pressure to afford the desired 1-azido-12-methyl-3,6,9-trioxa-12-azapentadecan-15-amine (1.9 g, 90.0% purity, 5.91 mmol, 95.9% yield). The crude product obtained was used in further experiments without any additional purification.

Step 6: The synthesis of 4-(16-azido-5-methyl-8,11,14-trioxa-1,5-diazahexadecan-1-yl)-2-{[(4-tert-butylcyclohexyl)(methyl)amino]methyl}quinazoline. The starting 1-azido-12-methyl-3,6,9-trioxa-12-azapentadecan-15-amine (530.0 mg, 1.83 mmol), 2-[(4-tert-butylcyclohexyl)(methyl)amino]methylquinazolin-4-ol (545.21 mg, 1.66 mmol) and (1H-1,2,3-benzotriazol-1-yloxy)tris(dimethylamino)phosphonium hexafluorophosphanuide (957.28 mg, 2.16 mmol) were mixed together in dry DMF (40 ml), after that 1,8-diazabicyclo[5.4.0]undec-7-ene (887.12 mg, 5.83 mmol, 870.0 μl, 3.5 eq) was added to the resulting solution, which was then left while stirring overnight at room temperature. After 12 hours the mixture was concentrated under reduced pressure and the crude residue obtained was diluted with distilled water (30 ml) and extracted with MtBE (3×30 ml). The organic layers were combined, washed with water (50 ml), brine (50 ml), dried over anhydrous Na₂SO₄ and filtered. The filtrate collected was concentrated under reduced pressure and the crude product obtained was subjected for flash chromatography purification (Interchim; 40 g SiO2, MtBE/methanol with methanol from 0˜95%, flow rate=40 ml/min, Rf=10-20 CV) to result in the desired 4-(16-azido-5-methyl-8,11,14-trioxa-1,5-diazahexadecan-1-yl)-2-[(4-tert-butylcyclohexyl)(methyl)amino]methylquinazoline (0.27 g, 27.1% yield). LCMS (2 min, 100% by UV 215 nm, RT 1.063, (M+)=599.2).

Step 7: The synthesis of (2S,4R)-1-[(2S)-2-[4-(1-{15-[(2-{[(4-tert-butylcyclohexyl)(methyl)amino]methyl}quinazolin-4-yl)amino]-12-methyl-3,6,9-trioxa-12-azapentadecan-1-yl}-1H-1,2,3-triazol-4-yl)butanamido]-3,3-dimethylbutanoyl]-4-hydroxy-N-{[4-(4-methyl-1,3-thiazol-5-yl)phenyl]methyl}pyrrolidine-2-carboxamide (Z5454478682). The starting (2S,4R)-1-[(2S)-2-(hex-5-ynamido)-3,3-dimethylbutanoyl]-4-hydroxy-N-[4-(4-methyl-1,3-thiazol-5-yl)phenyl]methylpyrrolidine-2-carboxamide (65.71 mg, 125.23 gmol) was dissolved in the mixture of t-BuOH (10 ml) and distilled water (2 ml) and the resulting solution was cooled down to 0° C. After that sodium (2R)-2-[(1S)-1,2-dihydroxyethyl]-4-hydroxy-5-oxo-2,5-dihydrofuran-3-olate (33.08 mg, 166.98 gmol) was added to the resulting solution, followed by the addition of copper (II) sulfate pentahydrate (6.25 mg, 25.05 gmol) and in 5 minutes 4-(16-azido-5-methyl-8,11,14-trioxa-1,5-diazahexadecan-1-yl)-2-[(4-tert-butylcyclohexyl)(methyl)amino]methylquinazoline (50.0 mg, 83.49 μmol). The resulting reaction mixture was then left while stirring at 50° C. overnight. After 15 hours full conversion of the starting material into the desired product was detected by LCMS, so the reaction mixture was concentrated under reduced pressure and the residue obtained was dissolved in ethyl acetate (50 ml), washed with water (2×35 ml) and saturated brine (35 ml). The organic layer was separated, dried over anhydrous Na2SO4 and filtered. The filtrate collected was concentrated under reduced pressure and the crude residue obtained was subjected for prep HPLC purification to result in the desired (2S,4R)-1-[(2S)-2-[4-(1-15-[(2-[(4-tert-butylcyclohexyl)(methyl)amino]methylquinazolin-4-yl)amino]-12-methyl-3,6,9-trioxa-12-azapentadecan-1-yl-1H-1,2,3-triazol-4-yl)butanamido]-3,3-dimethylbutanoyl]-4-hydroxy-N-[4-(4-methyl-1,3-thiazol-5-yl)phenyl]methylpyrrolidine-2-carboxamide (14.2 mg, 95.0% purity, 12.01 gmol, 14.4% yield). LCMS (2.5 min, 100% by UV 215 nm, RT 1.145, (M+)=1124.4).

### Example 12: Synthesis of N-{15-[(2-{[(4-tert-butylcyclohexyl)(methyl)amino]methyl}quinazolin-4-yl)amino]-12-methyl-3,6,9-trioxa-12-azapentadecan-1-yl}acetamide (Z5454478690)

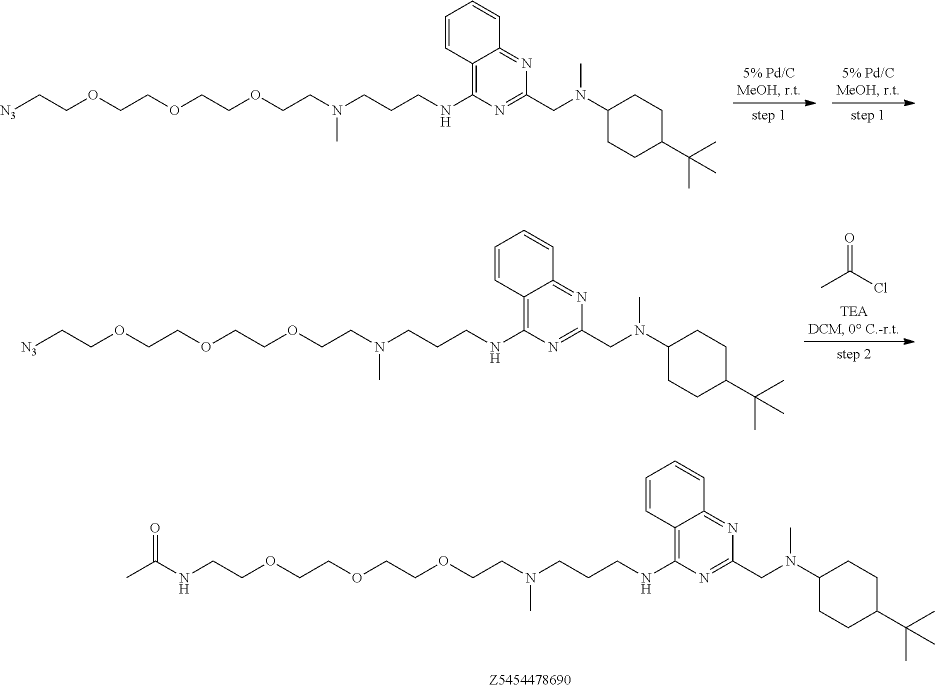

Step 1: The synthesis of N15-(2-{[(4-tert-butylcyclohexyl)(methyl)amino]methyl}quinazolin-4-yl)-12-methyl-3,6,9-trioxa-12-azapentadecane-1,15-diamine. The starting 4-(16-azido-5-methyl-8,11,14-trioxa-1,5-diazahexadecan-1-yl)-2-[(4-tert-butylcyclohexyl)(methyl)amino]methylquinazoline (250.0 mg, 417.49 gmol) was dissolved in absolute methanol (10 ml), after that 5% palladium on carbon (8.88 mg, 83.45 gmol) was added to the resulting solution. The reaction mixture was then degassed and flushed with H2 three times. The mixture was hydrogenated (1 atm) at room temperature overnight. After 18 hours full conversion of starting material was detected by LCMS, so the reaction mixture was filtered through a celite pad, washed with methanol (5 ml) and the filtrates collected were combined and concentrated under reduced pressure to result in the desired N15-(2-[(4-tert-butylcyclohexyl)(methyl)amino]methylquinazolin-4-yl)-12-methyl-3,6,9-trioxa-12-azapentadecane-1,15-diamine (200.0 mg, 95.0% purity, 331.69 gmol, 79.5% yield). LCMS (2 min, 95.3% by UV 215 nm, RT 0.872, (M+)=573.4). The product obtained used in further experiments without any additional purification.

Step 2: The synthesis of N-{15-[(2-{[(4-tert-butylcyclohexyl)(methyl)amino]methyl}quinazolin-4-yl)amino]-12-methyl-3,6,9-trioxa-12-azapentadecan-1-yl}acetamide (Z5454478690). The starting N15-(2-[(4-tert-butylcyclohexyl)(methyl)amino]methylquinazolin-4-yl)-12-methyl-3,6,9-trioxa-12-azapentadecane-1,15-diamine (30.0 mg, 52.37 gmol) was dissolved in dichloromethane (10 ml), and triethylamine (15.9 mg, 157.11 μmol, 20.0 μl, 3.0 eq) was added to the resulting solution, which was then cooled down with an ice bath. After that the solution of acetyl chloride (4.11 mg, 52.37 μmol, 1.0 eq) in dichloromethane (5 ml) was added dropwise at 0° C. during 15 minutes. After the addition was completed the reaction mixture was allowed to warm up to room temperature and left while stirring overnight. After 14 hours the mixture was diluted with DCM (30 ml) and washed with water (20 ml) and saturated aqueous solution of NaHCO₃ (20 ml). The organic layer was separated, dried over anhydrous Na₂SO₄ and filtered. The filtrate collected was concentrated under reduced pressure and the residue obtained was subjected for prep HPLC (35-80% 0-5 min H2O/acetonitrile flow: 30 ml/min) to result in the desired N-15-[(2-[(4-tert-butylcyclohexyl)(methyl)amino]methylquinazolin-4-yl)amino]-12-methyl-3,6,9-trioxa-12-azapentadecan-1-ylacetamide (11.3 mg, 18.38 mol, 35.1% yield). LCMS (2 min, 100% by UV 215 nm, RT 0.973, (M+)=616.4).

### Example 13: Synthesis of N-{15-[(2-{[(4-tert-butylcyclohexyl)(methyl)amino]methyl}quinazolin-4-yl)amino]-12-methyl-3,6,9-trioxa-12-azapentadecan-1-yl}-2-{[2-(2,6-dioxopiperidin-3-yl)-1-oxo-2,3-dihydro-1H-isoindol-4-yl]amino}acetamide (Z5592320713)

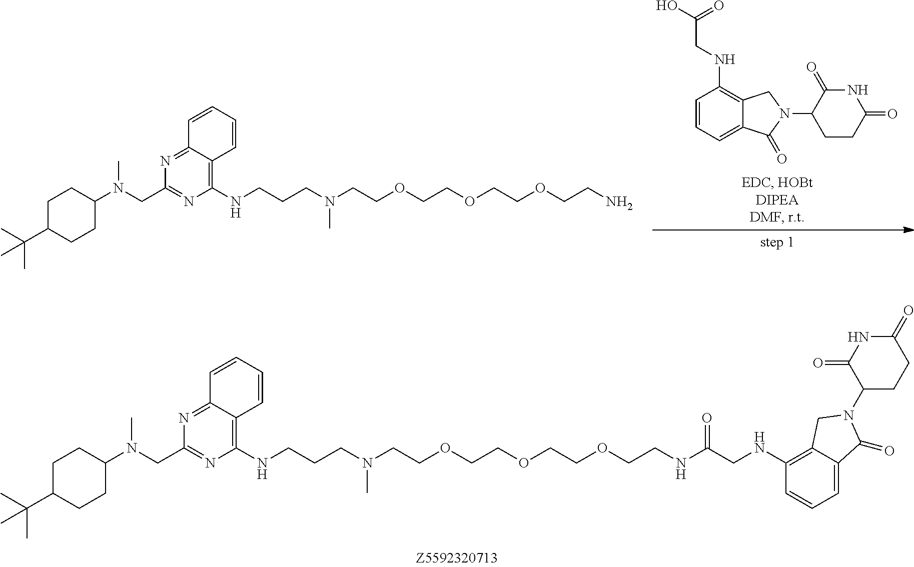 [SMILES: CN(CCCNc1nc(CN(C)C2CCC(C(C)(C)C)CC2)nc2ccccc12)CCOCCOCCOCCN ; O=C(O)CNc1cccc2c1CN(C1CCC(=O)NC1=O)C2=O ; CN(CCCNc1nc(CN(C)C2CCC(C(C)(C)C)CC2)nc2ccccc12)CCOCCOCCOCCNC(=O)CNc1cccc2c1CN(C1CCC(=O)NC1=O)C2=O]

Step 1: The synthesis of N-{15-[(2-{[(4-tert-butylcyclohexyl)(methyl)amino]methyl}quinazolin-4-yl)amino]-12-methyl-3,6,9-trioxa-12-azapentadecan-1-yl}-2-{[2-(2,6-dioxopiperidin-3-yl)-1-oxo-2,3-dihydro-1H-isoindol-4-yl]amino}acetamide (Z5592320713). To a solution of 2-[2-(2,6-dioxopiperidin-3-yl)-1-oxo-2,3-dihydro-1H-isoindol-4-yl]aminoacetic acid (16.77 mg, 52.86 μmol) in dry DMF (10 ml) at 0° C. was added 1-hydroxybenzotriazole (10.71 mg, 79.29 μmol), followed by the addition of N-(3-dimethylaminopropyl)-N′-ethylcarbodiimide hydrochloride (20.27 mg, 105.72 μmol), ethylbis(propan-2-yl)amine (34.13 mg, 264.09 μmol, 50.0 μl, 5.0 eq) and after 5 min N15-(2-[(4-tert-butylcyclohexyl)(methyl)amino]methylquinazolin-4-yl)-12-methyl-3,6,9-trioxa-12-azapentadecane-1,15-diamine* (30.28 mg, 52.86 μmol). The resulting reaction mixture was left while stirring at room temperature overnight. After 16 hours the mixture was concentrated under reduced pressure and the residue obtained was diluted with DCM (50 ml), washed with water (50 ml), saturated aqueous solution of NaHCO3 (30 ml) and brine (30 ml). The organic layer was separated, dried over anhydrous Na2SO4 and filtered. The filtrate collected was concentrated under reduced pressure to afford crude material that was subjected for prep HPLC purification (10-35% 0.5-6.5 min; 30 ml/min water-acetonitrile+TFA; column SunFire C18 19*100 mm) to result in the desired N-15-[(2-[(4-tert-butylcyclohexyl)(methyl)amino]methylquinazolin-4-yl)amino]-12-methyl-3,6,9-trioxa-12-azapentadecan-1-yl-2-[2-(2,6-dioxopiperidin-3-yl)-1-oxo-2,3-dihydro-1H-isoindol-4-yl]aminoacetamide (15.9 mg, 95.0% purity, 17.32 μmol, 32.8% yield). LCMS (2 min, 97.33% by UV 215 nm, RT 0.945, (M+)=872.4).

### Example 14: Synthesis of 4-(19-azido-5-methyl-8,11,14,17-tetraoxa-1,5-diazanonadecan-1-yl)-2-({4-[bis(4-chlorophenyl)methyl]piperazin-1-yl}methyl)quinazoline (Z4368602827)

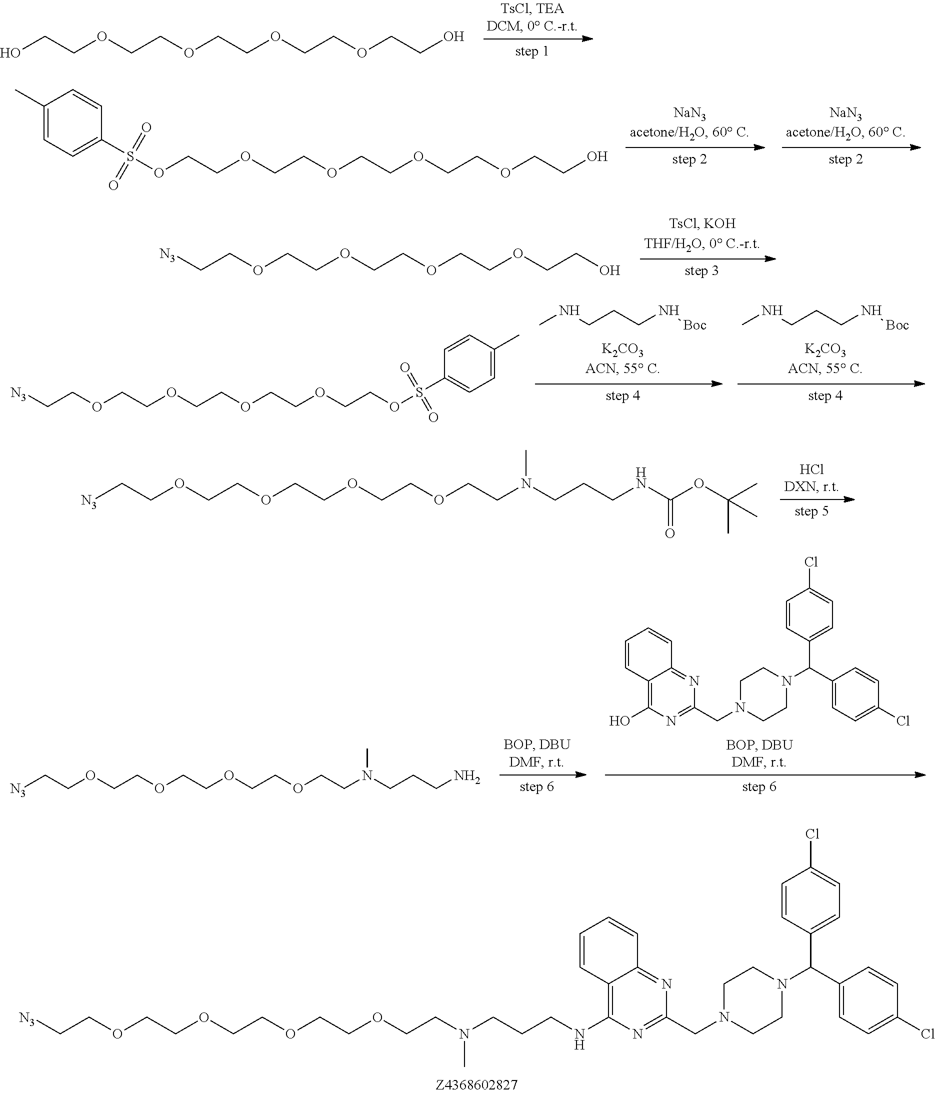

Step 1: The synthesis of 14-[(4-methylbenzenesulfonyl)oxy]-3,6,9,12-tetraoxatetradecan-1-ol. The reaction was carried out in 2 portions. The procedure described below was followed for each portion. The starting 3,6,9,12-tetraoxatetradecane-1,14-diol (200.0 g, 839.36 mmol, 177.62 ml, 3.33 eq) was dissolved in dry dichloromethane (3000 ml), then triethylamine (38.22 g, 377.71 mmol, 52.65 ml, 1.5 eq) was added and the resulting mixture was cooled down to 0° C. The solution of 4-methylbenzene-1-sulfonyl chloride (48.01 g, 251.81 mmol) in dry dichloromethane (2000 ml) was slowly added dropwise to the reaction mixture maintaining the temperature below 5° C. The reaction mixture was then allowed to warm up to room temperature and left while stirring for 2 days. After 48 hours the mixture was washed with distilled water (3×1000 ml). The organic layer was separated, dried over anhydrous Na2SO4 and filtered. The filtrate collected was concentrated under reduced pressure to afford 69 g of crude oily residue, which was purified by flash column chromatography (Interchim, 800 g SiO2, chloroform/acetonitrile with acetonitrile from 0˜95%, flow rate=125 ml/min, Rf=6-14 CV) to result in the desired 14-[(4-methylbenzenesulfonyl)oxy]-3,6,9,12-tetraoxatetradecan-1-ol (50.0 g, 95.0% purity, 121.03 mmol, 48.1% yield) as pale yellow oil; LCMS (2 min, 100% by UV 215 nm, RT 1.137, (M+)=393.2).

Step 2: The synthesis of 14-azido-3,6,9,12-tetraoxatetradecan-1-ol. The starting 14-[(4-methylbenzenesulfonyl)oxy]-3,6,9,12-tetraoxatetradecan-1-ol (100.0 g, 254.81 mmol) was dissolved in acetone (750 ml), after that the solution of sodium azide (82.82 g, 1.27 mol, 5.0 eq) in water (250 ml) was added and the resulting mixture was heated up to 60° C. and left while stirring overnight. After 15 hours the mixture was concentrated under reduced pressure and the residue obtained was diluted with distilled water (500 ml) and extracted with dichloromethane (3×200 ml). The organic layers were combined, dried over anhydrous Na2SO4 and filtered. The filtrate collected was concentrated under reduced pressure to afford the desired 14-azido-3,6,9,12-tetraoxatetradecan-1-ol (60.0 g, 90.0% purity, 205.1 mmol, 80.5% yield); LCMS (2 min, 100% by UV 215 nm, RT 0.846, (M+)=264.0). The crude product obtained was additionally dried under high vacuum and used in further experiments without any extra purification.

Step 3: The synthesis of 1-azido-14-[(4-methylbenzenesulfonyl)oxy]-3,6,9,12-tetraoxatetradecane. A solution of both 14-azido-3,6,9,12-tetraoxatetradecan-1-ol (60.0 g, 227.88 mmol) and 4-methylbenzene-1-sulfonyl chloride (65.17 g, 341.83 mmol) in THF (800 ml) was cooled down with an ice/water bath. After that the solution of potassium hydroxide (38.36 g, 683.66 mmol) in distilled water (80 ml) was added dropwise to the resulting solution. After the addition was completed the reaction mixture was allowed to warm up to room temperature and left while stirring overnight. After 15 hours the reaction mixture was concentrated under reduced pressure and the crude residue obtained was dissolved in DCM (1000 ml) and washed with water (2×500 ml). The organic layer was separated, dried over anhydrous Na2SO4 and filtered. The filtrate collected was concentrated under reduced pressure to afford the desired 1-azido-14-[(4-methylbenzenesulfonyl)oxy]-3,6,9,12-tetraoxatetradecane (77.0 g, 95.0% purity, 175.22 mmol, 76.9% yield): LCMS (2 min, 100% by UV 215 nm, RT 1.366, (M−N2+)=390.2). The crude material was used in further experiments without any additional purification.

Step 4: The synthesis of tert-butyl N-(1-azido-15-methyl-3,6,9,12-tetraoxa-15-azaoctadecan-18-yl)carbamate. The starting 1-azido-14-[(4-methylbenzenesulfonyl)oxy]-3,6,9,12-tetraoxatetradecane (59.61 g, 142.8 mmol) and tert-butyl N-[3-(methylamino)propyl]carbamate (24.44 g, 129.82 mmol) were dissolved in dry acetonitrile (600 ml), after that potassium carbonate (53.82 g, 389.45 mmol) was added to the resulting solution. The reaction mixture was then left while vigorous stirring overnight at 55° C. After 15 hours the solvent was removed by evaporation. Ethyl acetate (500 ml) was added to the residue obtained and washed with water (3×200 ml). The organic layer was separated, dried over anhydrous sodium sulfate and filtered. The filtrate collected was concentrated under reduced pressure to afford 62 g of crude oily residue, which was subjected for FC purification (Interchim; 800 g SiO2, MtBE/methanol with methanol from 0˜95%, flow rate=150 ml/min, Rt=30 min) to result in the desired tert-butyl N-(1-azido-15-methyl-3,6,9,12-tetraoxa-15-azaoctadecan-18-yl)carbamate (35.0 g, 95.0% purity, 76.69 mmol, 59.1% yield) as white oil: LCMS (2 min, RT 1.087, (M+)=434.2.

Step 5: The synthesis of 1-azido-15-methyl-3,6,9,12-tetraoxa-15-azaoctadecan-18-amine. The starting tert-butyl N-(1-azido-15-methyl-3,6,9,12-tetraoxa-15-azaoctadecan-18-yl)carbamate (25.0 g, 57.66 mmol) was dissolved in absolute methanol (200 ml) and 3M hydrogen chloride in 1,4-dioxane (21.03 g, 576.65 mmol, 192.22 ml, 10.0 eq) was added to the resulting solution. The reaction mixture was then left while stirring overnight at room temperature. After 12 hours the reaction mixture was concentrated under reduced pressure to dryness and the residue obtained was dissolved in 50% aqueous solution of KOH (300 ml) and extracted with DCM (3×200 ml). The organic layers were combined, dried over anhydrous sodium sulfate and filtered. The filtrate collected was concentrated under reduced pressure to afford the desired 1-azido-15-methyl-3,6,9,12-tetraoxa-15-azaoctadecan-18-amine (17.0 g, 90.0% purity, 45.89 mmol, 79.6% yield): LCMS (2 min, 100% by UV 215 nm, RT 0.592, (M+)=334.2); The crude product was used in further experiments without any additional purification.

Step 6: The synthesis of 4-(19-azido-5-methyl-8,11,14,17-tetraoxa-1,5-diazanonadecan-1-yl)-2-({4-[bis(4-chlorophenyl)methyl]piperazin-1-yl}methyl)quinazoline (Z4368602827). The starting 2-(4-[bis(4-chlorophenyl)methyl]piperazin-1-ylmethyl)quinazolin-4-ol (1.0 g, 2.09 mmol), (1H-1,2,3-benzotriazol-1-yloxy)tris(dimethylamino)phosphanium hexafluorophosphanuide (1.2 g, 2.71 mmol) and DBU (1.43 g, 9.39 mmol) were mixed together in dry DMF (80 ml), after that 1-azido-15-methyl-3,6,9,12-tetraoxa-15-azaoctadecan-18-amine (1.04 g, 3.13 mmol) was added to the resulting reaction mixture solution, which was then left while stirring overnight. After 12 hours the mixture was concentrated under reduced pressure and the crude residue obtained was diluted with distilled water (60 ml) and extracted with MtBE (3×30 ml). The organic layers were combined, washed with water (3×20 ml), brine (50 ml), dried over anhydrous Na2SO4 and filtered. The filtrate collected was concentrated under reduced pressure to afford 1.7 g of crude product. The material obtained was subjected for FC purification (Interchim; 40 g SiO2, MtBE/methanol with methanol from 0˜95%, flow rate=40 ml/min, Rf=7-12 CV) to result in the desired 4-(19-azido-5-methyl-8,11,14,17-tetraoxa-1,5-diazanonadecan-1-yl)-2-(4-[bis(4-chlorophenyl)methyl]piperazin-1-ylmethyl)quinazoline (350.0 mg, 95.0% purity, 418.34 μmol, 20.1% yield): LCMS (2 min, 100% by UV 215 nm, RT 1.227, (M+)=794.2).

### Example 15: Synthesis of 4-(16-azido-5-methyl-8,11,14-trioxa-1,5-diazahexadecan-1-yl)-2-({4-[bis(4-chlorophenyl)methyl]piperazin-1-yl}methyl)quinazoline (Z4437118918)

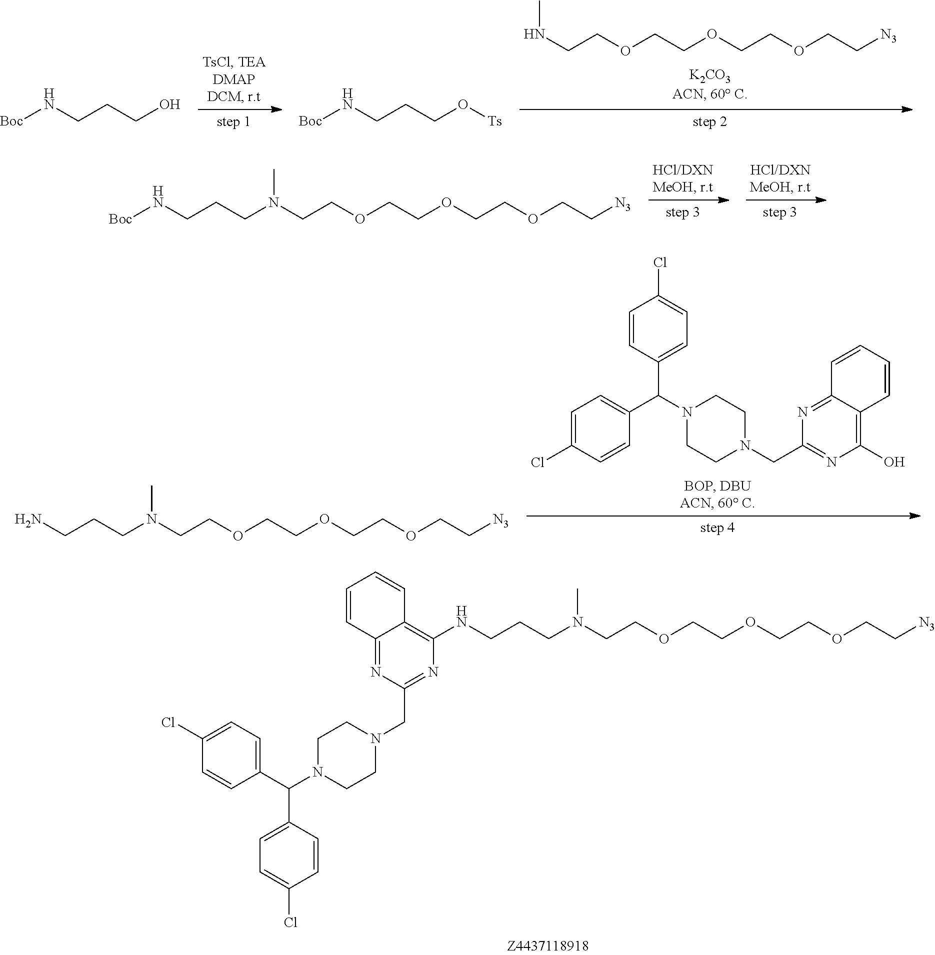

Step 1: The synthesis of tert-butyl N-{3-[(4-methylbenzenesulfonyl)oxy]propyl}carbamate. Tert-butyl N-(3-hydroxypropyl)carbamate (8.0 g, 45.66 mmol), triethylamine (5.54 g, 54.78 mmol, 7.64 ml, 1.2 eq) and N,N-dimethylpyridin-4-amine (557.75 mg, 4.57 mmol) were mixed together in DCM (500 ml) and the resulting solution was stirred for about 5 minutes. After that period 4-Methylbenzene-1-sulfonyl chloride (9.57 g, 50.22 mmol) was added to the reaction mixture, which was left while stirring at room temperature for night. After 12 hours the reaction progress was checked by NMR that showed 85% conversion of the starting material into the desired product. The reaction mixture was washed with distilled water (3×400 ml). The organic layer was separated, dried over anhydrous sodium sulfate and concentrated under reduced pressure to afford 12.8 g of crude product which was sent for FC purification (ISCO® Companion combiflash; 220 g SiO2, CHCl3/CH3CN with CH3CN from 0˜95%, flow rate=100 mL/min, RT=23 min) resulting in the desired tert-butyl N-3-[(4-methylbenzenesulfonyl)oxy]propylcarbamate (6.85 g, 19.75 mmol, 43.3% yield): LCMS (2 min, 95,9% by UV 215 nm, RT 1.286, M-Boc+1=230.0); The crude product was used in further experiments.

Step 2: The synthesis of tert-butyl N-(1-azido-12-methyl-3,6,9-trioxa-12-azapentadecan-15-yl)carbamate. The starting 13-azido-5,8,11-trioxa-2-azatridecane (1.4 g, 6.03 mmol) was dissolved in dry acetonitrile (100 ml), after that potassium carbonate (2.5 g, 18.09 mmol) was added to the resulting solution, followed by the addition of tert-butyl N-3-[(4-methylbenzenesulfonyl)oxy]propylcarbamate (2.18 g, 6.63 mmol). The reaction mixture was then heated up to 60° C. and left while stirring overnight. After 12 hours the reaction mass was analyzed with NMR that showed full conversion of the starting material, as well as the presence of the desired product. So the solvent was removed by evaporation and the residue obtained was diluted with DCM (200 ml). The resulting solution was washed with 50% aqueous solution of KOH (150 ml). The organic layer was separated, dried over anhydrous sodium sulfate and concentrated under reduced pressure to afford the desired tert-butyl N-(1-azido-12-methyl-3,6,9-trioxa-12-azapentadecan-15-yl)carbamate (1.9 g, 2.44 mmol, 40.5% yield) as yellow oil. LCMS (2 min, NI by UV 215 nm, RT 0.891, M+1=390.2). The crude obtained was used in further experiments without any additional purification.

Step 3: The synthesis of 1-azido-12-methyl-3,6,9-trioxa-12-azapentadecan-15-amine. Tert-butyl N-(1-azido-12-methyl-3,6,9-trioxa-12-azapentadecan-15-yl)carbamate (1.4 g, 3.59 mmol) was dissolved in absolute methanol (30 ml), after that hydrogen chloride in 1,4-dioxane, 10% (1.31 g, 35.93 mmol, 1, 10.0 eq) was added to the resulting solution. The reaction mass was then left while stirring overnight at room temperature. After 12 hours the reaction mixture was concentrated under reduced pressure and the residue obtained was dissolved in 50% aqueous solution of KOH (100 ml) and extracted with DCM (2×100 ml). The organic layers were combined, dried over anhydrous sodium sulfate and concentrated under reduced pressure to afford the desired 1-azido-12-methyl-3,6,9-trioxa-12-azapentadecan-15-amine (700 mg, 1.69 mmol, 47.1% yield): LCMS (2 min, NI by UV 215 nm, RT 0.379, M+1=290.2); The product obtained in this experiment was crude; however, it was used in further reaction steps without any additional purification.

Step 4: The synthesis of 4-(16-azido-5-methyl-8,11,14-trioxa-1,5-diazahexadecan-1-yl)-2-({4-[bis(4-chlorophenyl)methyl]piperazin-1-yl}methyl)quinazoline (Z4437118918). The starting 2-(4-[Bis(4-chlorophenyl)methyl]piperazin-1-ylmethyl)quinazolin-4-ol-common intermediate, the synthesis of which described in the synthetic report “WXB2 N3”, step 6 (1.16 g, 2.42 mmol) and (1H-1,2,3-benzotriazol-1-yloxy)tris(dimethylamino)phosphonium hexafluorophosphate (1.39 g, 3.14 mmol) were suspended in dry DMF (40 ml). After that 1,8-diazabicyclo[5.4.0]undec-7-ene (1.29 g, 8.47 mmol, 1.27 ml, 3.5 eq) was added dropwise to the reaction mixture, which became homogeneous after the addition was completed. After 10 minute stirring at room temperature, 1-azido-12-methyl-3,6,9-trioxa-12-azapentadecan-15-amine (700.0 mg, 2.42 mmol) was added to the reaction mass solution, which was then stirred for further 16 hours at room temperature. The next day the reaction mass was analyzed with LCMS that showed 25% conversion of the starting material into the desired product. DMF was evaporated in vacuum to dryness and the residue obtained was diluted with ethyl acetate (500 ml). The solution obtained was washed with hot water (5×300 ml). The organic layer was then separated, dried over anhydrous sodium sulfate and concentrated under reduced pressure to afford 1.3 g of crude product as black oil which was purified through prep HPLC (10-45% 2-7 min water-acetonitrile; flow 30 ml/min; (loading pump 4 ml/min acetonitrile); column SunFireC18 100×19 mm 5 um) to afford the desired 4-(16-azido-5-methyl-8,11,14-trioxa-1,5-diazahexadecan-1-yl)-2-(4-[bis(4-chlorophenyl)methyl]piperazin-1-ylmethyl)quinazoline (440.0 mg, 556.77 μmol, 23% yield). LCMS (2.5 min, 100% by UV 215 nm, RT 1.111, M+1=750.3).

### Example 16: Synthesis of 2-{[4-({4-[(14-azido-3,6,9,12-tetraoxatetradecan-1-yl)oxy]phenyl}(4-chlorophenyl)methyl)piperazin-1-yl]methyl}-N-[3-(dimethylamino)propyl]quinazolin-4-amine (Z4431078002)

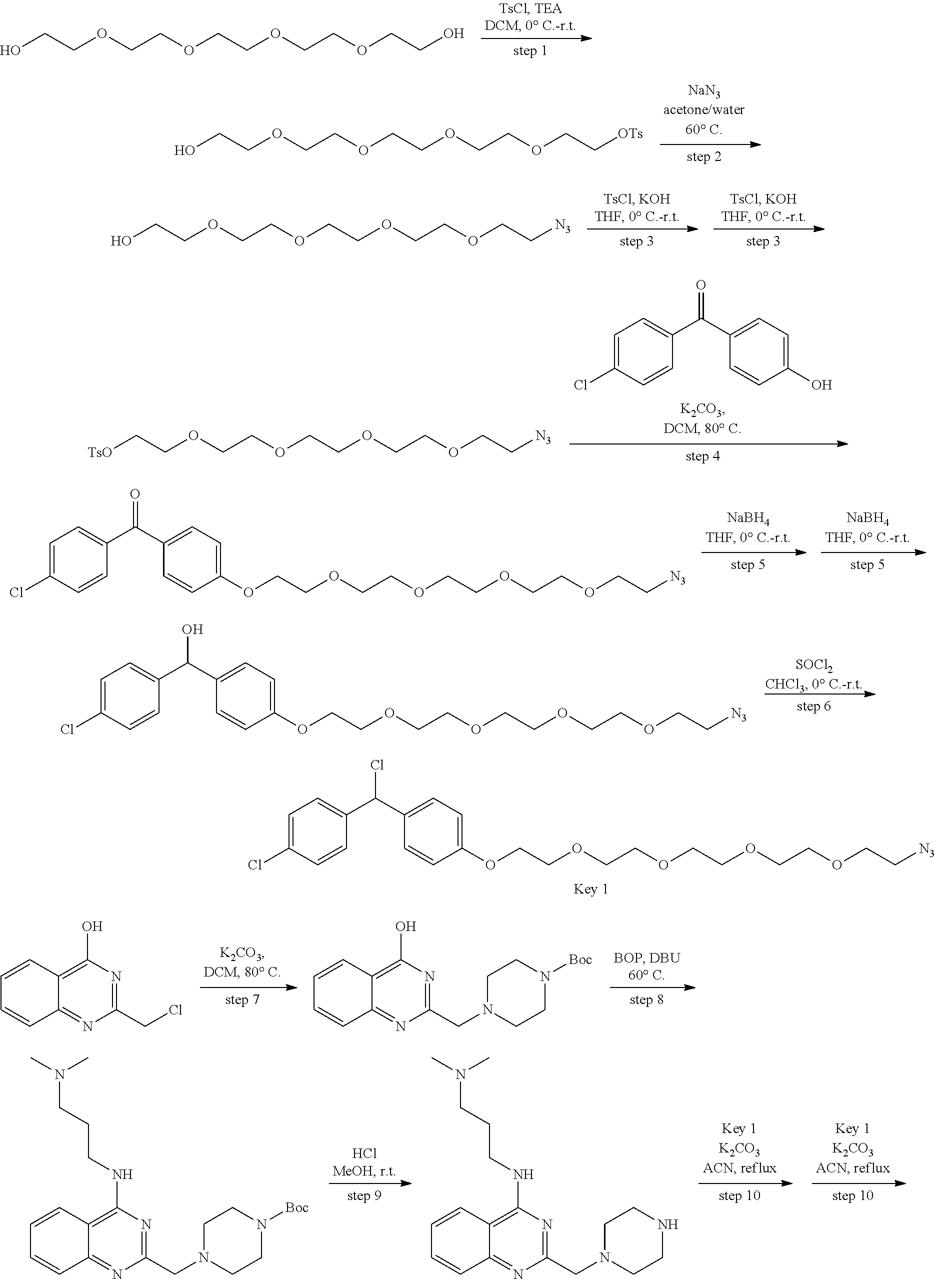

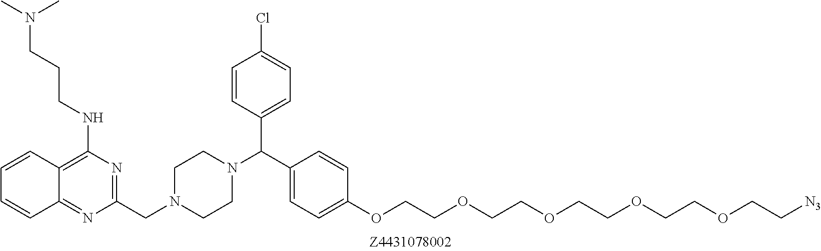 [SMILES: CN(C)CCCNc1nc(CN2CCN(C(c3ccc(Cl)cc3)c3ccc(OCCOCCOCCOCCOCCN=[N+]=[N-])cc3)CC2)nc2ccccc12]

Step 1: The synthesis of 14-[(4-methylbenzenesulfonyl)oxy]-3,6,9,12-tetraoxatetradecan-1-ol. The starting 3,6,9,12-tetraoxatetradecane-1,14-diol (100.0 g, 419.68 mmol) was dissolved in dry DCM (1600 mL), after that triethylamine (19.11 g, 188.86 mmol, 26.32 ml) was added to the resulting solution, which was cooled down to 0° C. with an ice bath. Then ⅓ of total amount of 4-methylbenzene-1-sulfonyl chloride (24.0 g, 125.9 mmol) in dry DCM (800 ml) was slowly added dropwise to the reaction mixture at 0° C. (over the period of 4 hours). The next day the completion of the reaction was checked by NMR (P783242-1) that showed the presence of the starting material. So ⅓ of total amount of 4-methylbenzene-1-sulfonyl chloride (24.0 g, 125.9 mmol) dissolved in DCM (800 ml) was slowly added dropwise at 0° C. to the cooled reaction mixture over the period of 4 hours. The next day the aliquot was taken again and analyzed with NMR (P783242-21) that showed that the conversion was still not full. So another ⅓ portion of total amount of 4-methylbenzene-1-sulfonyl chloride (24.0 g, 125.9 mmol) in DCM (800 ml) was slowly added dropwise at the temperature of 0° C. during 4 hour period. The mixture was then allowed to warm up to room temperature and left while stirring overnight. After 12 hours the reaction mixture was washed with water (3×2000 ml). The organic layer was separated, dried over anhydrous Na2SO4 and concentrated under reduced pressure to afford the desired 14-[(4-methylbenzenesulfonyl)oxy]-3,6,9,12-tetraoxatetradecan-1-ol (35.0 g, 74.91 mmol, 59.5% yield): LCMS (2 min, 100% by UV 215 mM, RT 1.208, M+1=393.0). The product obtained was used in the further experiments without any additional purification.

Step 2: The synthesis of 14-azido-3,6,9,12-tetraoxatetradecan-1-ol. 14-[(4-Methylbenzenesulfonyl)oxy]-3,6,9,12-tetraoxatetradecan-1-ol (87.8 g, 223.71 mmol) was dissolved in the mixture of acetone (750 ml) and distilled water (250 ml), after that sodium azide (72.72 g, 1.12 mol) was added and the resulting reaction mixture was then heated up to 60° C. and left while stirring at this temperature for night. The next day the reaction mass was analyzed with NMR (P866936-1) that showed full conversion of the starting material. The solvents were removed from the reaction mass by evaporation and the residue obtained was diluted with DCM (1000 ml) and washed with water (3×600 ml). The organic layer was dried over anhydrous Na2SO4 and concentrated under reduced pressure to afford the desired 14-azido-3,6,9,12-tetraoxatetradecan-1-ol (54 g, 184.59 mmol, 82.5% yield); LCMS (2 min, RT 0.863, M+1=264.2). The product obtained was used in the further experiments without any additional purification.

Step 3: The synthesis of 1-azido-14-[(4-methylbenzenesulfonyl)oxy]-3,6,9,12-tetraoxatetradecane. A solution of both 14-azido-3,6,9,12-tetraoxatetradecan-1-ol (54.0 g, 205.1 mmol) and 4-methylbenzene-1-sulfonyl chloride (58.65 g, 307.64 mmol) in THF (750 ml) was cooled with an ice/water bath. After that the solution of potassium hydroxide (34.52 g, 615.29 mmol) in water (10 ml) was added slowly via drops. After the addition was completed the reaction mixture was allowed to warm up to room temperature and left while stirring overnight. The next day the reaction mass was analyzed with NMR (P888738-1) that showed full conversion of the starting material. So the reaction mass was evaporated to dryness. The crude residue obtained was dissolved in DCM (1000 ml) and washed with water (3×500 ml). The organic layer was separated, dried over anhydrous Na2SO4 and concentrated under reduced pressure to afford the desired 1-azido-14-[(4-methylbenzenesulfonyl) oxy]-3,6,9,12-tetraoxatetradecane (85 g, 183.24 mmol, 89.3% yield): LCMS (2 min, 100% by UV 215 nm, RT 1.312, M=390.1). The product obtained was used in further experiments without any additional purification.

Step 4: The synthesis of 1-azido-14-[4-(4-chlorobenzoyl)phenoxy]-3,6,9,12-tetraoxatetradecane. The starting 1-azido-14-[(4-methylbenzenesulfonyl)oxy]-3,6,9,12-tetraoxatetradecane (3.59 g, 8.6 mmol) was dissolved in dry DMF (20 ml), after that 4-(4-chlorobenzoyl)phenol (2.0 g, 8.6 mmol) was added to the resulting solution, followed by the addition of potassium carbonate (1.78 g, 12.89 mmol). The reaction mixture was then heated up to 80° C. and left while stirring at this temperature for night. After 12 hours an aliquot was analyzed with LCMS that showed full conversion of the starting material, so the reaction mixture was poured onto water (100 ml) and extracted with ethyl acetate (2×100 ml). The organic layers were combined, washed with brine (3×100 ml), dried over anhydrous sodium sulfate and concentrated under reduced pressure to afford 3.1 g of the crude product as yellow oil with 68% purity according to LCMS. The crude material obtained was purified by FC (ISCO® Companion combiflash; 80 g SiO2, hexane/THF with THF from 0˜95%, flow rate=60 mL/min, Rt=12 min). Chromatography run is attached below. As a result, the desired 1-azido-14-[4-(4-chlorobenzoyl)phenoxy]-3,6,9,12-tetraoxatetradecane (1.36 g, 2.56 mmol, 29.8% yield) as yellow oil: LCMS (2 min, 100% by UV 215 nm, RT 1.511, M+1=478.2). The product obtained was used in further experiments without any additional purification.

Step 5: The synthesis of {4-[(14-azido-3,6,9,12-tetraoxatetradecan-1-yl)oxy]phenyl}(4-chlorophenyl)methanol. To the solution of 1-azido-14-[4-(4-chlorobenzoyl)phenoxy]-3,6,9,12-tetraoxatetradecane (1.26 g, 2.64 mmol) in the mixture of dry THF (10 ml) and absolute methanol (10 ml) sodium borohydride (199.43 mg, 5.27 mmol) was added portionwise at 0° C. After the addition was completed the reaction mass was allowed to warm up to room temperature and left while stirring overnight. After 2 days the reaction mixture was poured onto water (100 ml) and extracted with ethyl acetate (2×100 ml). The organic layers were combined, washed with brine (100 ml), dried over anhydrous sodium sulfate and concentrated under reduced pressure to afford the desired 4-[(14-azido-3,6,9,12-tetraoxatetradecan-1-yl)oxy]phenyl(4-chlorophenyl)methanol (1.1 g, 1.95 mmol, 73.9% yield) as yellow oil: LCMS (2 min, 100% by UV 215 nm, RT 1.281); The product obtained was used in further experiments without any additional purification.

Step 6: The synthesis of 1-azido-14-{4-[chloro(4-chlorophenyl)methyl]phenoxy}-3,6,9,12-tetraoxatetradecane. The starting 4-[(14-azido-3,6,9,12-tetraoxatetradecan-1-yl)oxy]phenyl(4-chlorophenyl)methanol (250.0 mg, 520.88 μmol) was dissolved in chloroform (10 ml), after that the resulting solution was cooled down to 0° C. with an ice bath. Thionyl chloride (310.32 mg, 2.61 mmol, 190.0 μl, 5.0 eq) was then added to the reaction mixture at 0° C. with one drop of DMF. The reaction mass was allowed to warm up to room temperature after the addition was completed and stirred overnight at room temperature. After 12 hour period an aliquot was taken from the reaction mass, evaporated in vacuum to dryness and analyzed with NMR that showed full conversion. So the reaction mass was concentrated under reduced pressure and the resulting semi-solid residue obtained was diluted with distilled water (50 ml) and extracted with ethyl acetate (2×50 ml). The organic layers were combined, washed with brine (50 ml), dried over anhydrous sodium sulfate and concentrated under reduced pressure to afford the desired 1-azido-14-4-[chloro(4-chlorophenyl)methyl]phenoxy-3,6,9,12-tetraoxatetradecane (200.0 mg, 321.03 μmol, 61.5% yield) as yellow oil: LCMS (2 min, 94.72% by UV 215 nm, RT 1.408, M+1=498.2); The crude product was used in further reaction step without any additional purification.

Step 7: The synthesis of tert-butyl 4-[(4-hydroxyquinazolin-2-yl)methyl]piperazine-1-carboxylate. The starting tert-butyl piperazine-1-carboxylate (5.0 g, 26.85 mmol) was dissolved in dry DMF (20 ml), after that 2-(chloromethyl)-3,4-dihydroquinazolin-4-one (5.33 g, 27.38 mmol) was added to the resulting solution, followed by the addition of potassium carbonate (7.42 g, 53.69 mmol). The reaction mixture was then heated up to 60° C. and left while stirring overnight. After 12 hours the reaction mass was analyzed by LCMS that showed full conversion of the starting material, as well as the presence of the desired product mass peak. So the reaction mixture was poured onto water (100 ml) and extracted with ethyl acetate (2×100 ml). The organic layers were combined, washed with brine (3×100 ml), dried over anhydrous sodium sulfate and concentrated under reduced pressure to afford 6.2 g of crude yellow solid of 80% purity according to the spectra data. The solid product obtained was triturated with MTBE and filtered to give the desired tert-butyl 4-[(4-hydroxyquinazolin-2-yl)methyl]piperazine-1-carboxylate (4.8 g, 12.54 mmol, 46.7% yield) as yellow solid: LCMS (2 min, 100% by UV 215 nm, RT 0.939, M+1=345.2); The product obtained was used in further experiments without any additional purification (the purity was sufficient).

Step 8: The synthesis of tert-butyl 4-[(4-{[3-(dimethylamino)propyl]amino}quinazolin-2-yl)methyl]piperazine-1-carboxylate. Tert-butyl 4-[(4-oxo-3,4-dihydroquinazolin-2-yl)methyl]piperazine-1-carboxylate (4.8 g, 13.94 mmol) and (1H-1,2,3-benzotriazol-1-yloxy)tris(dimethylamino)phosphonium hexafluorophosphate (8.01 g, 18.12 mmol) were suspended in acetonitrile (100 ml). After that 1,8-diazabicyclo[5.4.0]undec-7-ene (3.18 g, 20.91 mmol, 3.13 ml, 1.5 eq) was added dropwise to the reaction mixture, which was stirred at room temperature until became homogeneous. Then (3-aminopropyl)dimethylamine (2.14 g, 20.91 mmol, 2.61 ml, 1.5 eq) was also added dropwise and the resulting reaction mass solution was heated up to 60° C. and left while stirring overnight. After 12 hours the reaction mass was analyzed by LCMS that showed full conversion of the starting material, as well as the presence of the desired product. The reaction mixture was evaporated in vacuum to dryness and the residue obtained was diluted with distilled water (250 ml) and extracted several times with ethyl acetate (2×150 ml). The organic layers were combined, dried over anhydrous sodium sulfate and evaporated in vacuum to dryness to afford 6 g (10.78 mmol, 77.3% yield) of the desired crude product as brown oil with 77% purity according to the spectra data. The material obtained was purified by FC (ISCO® Companion combiflash; 120 g SiO2, MTBE/methanol with methanol from 0˜95%, flow rate=85 mL/min, Rt=19 min) to afford the desired tert-butyl 4-[(4-[3-(dimethylamino)propyl]aminoquinazolin-2-yl)methyl]piperazine-1-carboxylate (4.95 g) as brown oil: LCMS (2 min, 100% by UV 215 nm, RT 0.844, M+1=429.3).

Step 9: The synthesis of 4-[(4-{[3-(dimethylamino)propyl]amino}quinazolin-2-yl)methyl]piperazin-1-ium. Tert-butyl 4-[(4-[3-(dimethylamino)propyl]aminoquinazolin-2-yl)methyl]piperazine-1-carboxylate (7.0 g, 16.33 mmol) was dissolved in absolute methanol (70 ml), after that anhydrous hydrogen chloride, 10% solution in 1,4-dioxane (5.95 g, 163.33 mmol, 49.62 ml, 10.0 eq) was added dropwise. The resulting reaction mixture was then left while stirring at room temperature overnight. An aliquot was taken after 12 hour period and analyzed with NMR that showed full conversion of the starting material. The reaction mixture was concentrated in vacuum to dryness. The resulting solid residue was triturated with acetone (20 ml) and the precipitate formed was collected by filtration and dried to result in the desired 4-[(4-[3-(dimethylamino)propyl]aminoquinazolin-2-yl)methyl]piperazin-1-ium hydrochloride (7 g, 15.35 mmol, 94% yield) as yellow solid. LCMS (2 min, 100% by UV 215 nm, RT 0.137, M+1=329.2); The product obtained converted into its base form (diluted with 50% aqueous solution of KOH and extracted with DCM; organic layers combined, dried and concentrated under reduced pressure) directly before further experiments.

Step 10: The synthesis of 2-{[4-({4-[(14-azido-3,6,9,12-tetraoxatetradecan-1-yl)oxy]phenyl}(4-chlorophenyl)methyl)piperazin-1-yl]methyl}-N-[3-(dimethylamino)propyl]quinazolin-4-amine (Z4431078002). The starting N-[3-(dimethylamino)propyl]-2-[(piperazin-1-yl)methyl]quinazolin-4-amine (131.73 mg, 401.07 μmol) was dissolved in dry DMF (5 ml), after that 1-azido-14-4-[chloro(4-chlorophenyl)methyl]phenoxy-3,6,9,12-tetraoxatetradecane (200.0 mg, 401.28 μmol) was added to the resulting solution, followed by the addition of potassium carbonate (83.14 mg, 601.6 μmol). The reaction mixture was then heated up to 80° C. and left while stirring overnight. After 12 hours the reaction mass was analyzed by LCMS that showed full conversion of the starting material, as well as the presence of the desired product. The reaction mixture was poured onto water (100 ml) and extracted with ethyl acetate (2×100 ml). The organic layers were combined, washed with brine (3×100 ml), dried over anhydrous sodium sulfate and concentrated under reduced pressure to afford 0.26 g of crude product as yellow colored oil with 88% purity according to LCMS. The crude product obtained was purified by prep HPLC (60-100% 0.5-5 min water-MeOH+NH3; flow 30 ml/min (loading pump 4 ml/min MeOH+NH3); target mass 791; column ymc actus triat C18 100×19 mm 5 um) to afford 2-{[4-({4-[(14-azido-3,6,9,12-tetraoxatetradecan-1-yl)oxy]phenyl}(4-chlorophenyl)methyl)piperazin-1-yl]methyl}-N-[3-(dimethylamino)propyl]quinazolin-4-amine (120.2 mg, 144 μmol, 36% yield) as a yellow oil.

### Example 17: Synthesis of 3-{5-[2-oxo-4-(prop-2-enoyl)piperazin-1-yl]furan-2-yl}-N-[1-(2-{[2-(pyridin-3-yl)quinazolin-4-yl]amino}-2,3-dihydro-1H-inden-5-yl)-4,7,10,13-tetraoxaoctadec-1-yn-18-yl]propanamide (Z5431540269)

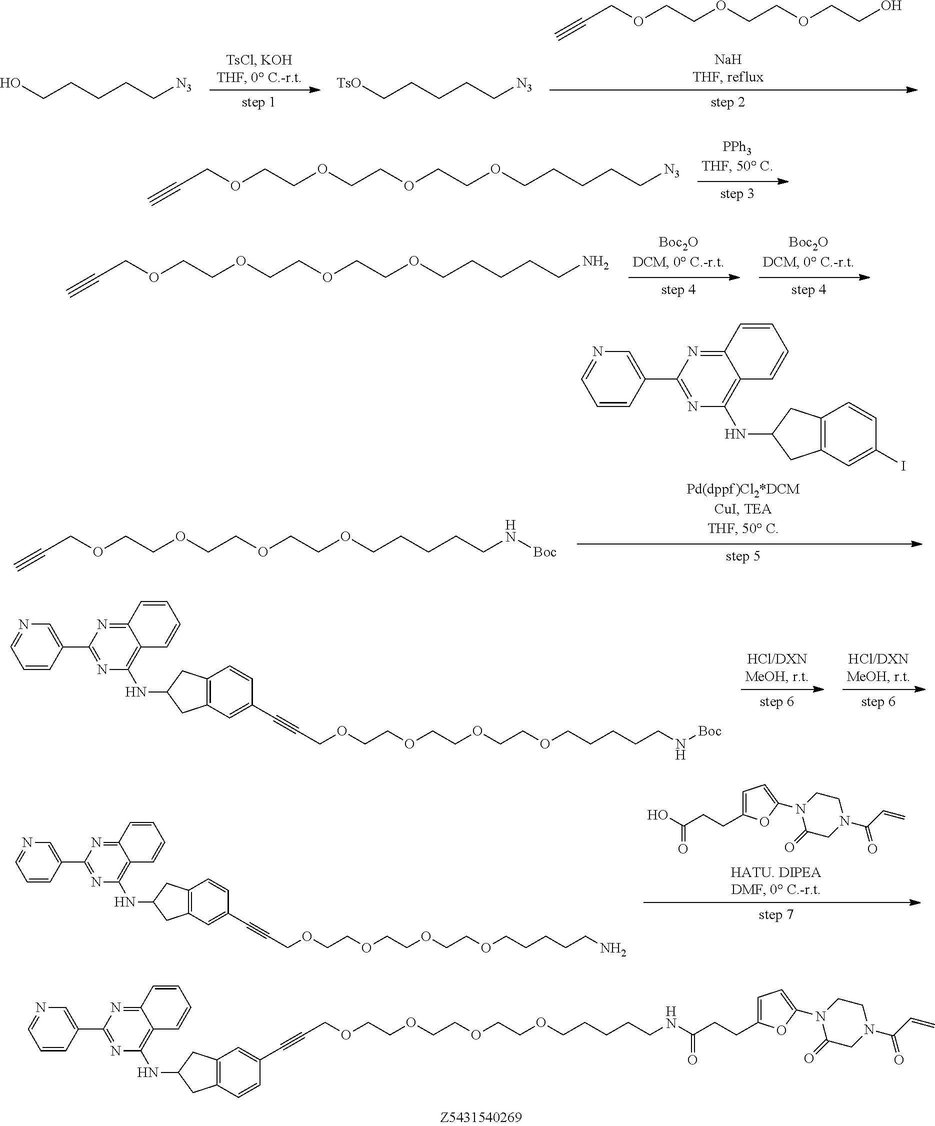

Step 1: The synthesis of 1-{[(5-azidopentyl)oxy]sulfonyl}-4-methylbenzene. A solution of both 5-azidopentan-1-ol (19.0 g, 147.1 mmol) and 4-methylbenzene-1-sulfonyl chloride (42.07 g, 220.65 mmol) in THF (800 ml) was cooled with an ice/water bath. After that the solution of potassium hydroxide (24.76 g, 441.31 mmol) in water (80 ml) was added dropwise. After the addition was completed the reaction mixture was allowed to warm up to room temperature and left while stirring overnight. After 15 hours the reaction mixture was evaporated to dryness. The crude residue obtained was dissolved in DCM (1000 ml) and washed with water (2×500 ml). The organic layer was separated, dried over anhydrous Na2SO4 and filtered. The filtrate collected was concentrated under reduced pressure to afford crude residue, which was subjected for flash chromatography purification (Interchim, 450 g SiO2, petroleum ether/MTBE with MTBE from 0˜95%, flow rate=100 mL/min, Rf=5.8 CV) to result in the desired 1-[(5-azidopentyl)oxy]sulfonyl-4-methylbenzene (26.0 g, 95.0% purity, 87.17 mmol, 59.3% yield). LCMS (2 min, 98.2% by UV 215 nm, RT=1.319, ((M−N2)+)=256).

Step 2: The synthesis of 18-azido-4,7,10,13-tetraoxaoctadec-1-yne. To a suspension of sodium hydride (1.41 g, 58.82 mmol) in anhydrous THF (100 ml), under inert atmosphere was added the solution of 2-2-[2-(prop-2-yn-1-yloxy)ethoxy]ethoxyethan-1-ol (7.31 g, 38.82 mmol) in THF (50 ml). The resulting suspension was stirred at room temperature for 30 minutes. After that period 1-[(5-azidopentyl)oxy]sulfonyl-4-methylbenzene (10.0 g, 35.29 mmol) was added dropwise and the resulting reaction mixture was heated to reflux and left while stirring overnight. After 18 hours the reaction mixture solution was diluted with H2O (100 ml) and ethyl acetate (100 ml), the layers were separated, the aqueous layer was additionally extracted with ethyl acetate (100 ml). The organic layers were combined, washed with brine (100 ml), dried over anhydrous Na2SO4 and filtered. The filtrate collected was concentrated under reduced pressure to afford crude residue, which was subjected for flash chromatography purification (Interchim, 220 g SiO2, petroleum ether/MTBE with MTBE from 0˜95%, flow rate=80 mL/min, Rt=35 min) to result in the desired 18-azido-4,7,10,13-tetraoxaoctadec-1-yne (3.8 g, 95.0% purity, 12.06 mmol, 34.2% yield).

Step 3: The synthesis of 4,7,10,13-tetraoxaoctadec-1-yn-18-amine. To a solution of triphenylphosphane (4.99 g, 19.04 mmol) in anhydrous THF (50 ml), under inert atmosphere was added the solution of 18-azido-4,7,10,13-tetraoxaoctadec-1-yne (3.8 g, 12.69 mmol) in THF (20 ml). The resulting suspension was stirred at 50° C. for 8 hours. Completion of the reaction was monitored by NMR analysis. After cooling to room temperature aqueous solution of HCl, 2M (50 ml) was added in one portion and the resulting mixture was stirred for 2 hours. After that period the organic solvent was removed by rotary evaporation, and the aqueous mixture obtained was extracted with MtBE (2×50 ml). The aqueous layer was separated, neutralized with aqueous solution of NaOH, 1OM (20 ml) and extracted with DCM (3×50 ml). DCM organic layers were combined, washed with brine (100 ml), dried over anhydrous Na2SO4 and filtered. The filtrate collected was concentrated under reduced pressure to afford 4,7,10,13-tetraoxaoctadec-1-yn-18-amine (3.2 g, 90.0% purity, 10.54 mmol, 83% yield). The product obtained was used in further experiments without any additional purification.

Step 4: The synthesis of tert-butyl N-(4,7,10,13-tetraoxaoctadec-1-yn-18-yl)carbamate. 4,7,10,13-Tetraoxaoctadec-1-yn-18-amine (3.2 g, 11.71 mmol) and triethylamine (1.78 g, 17.56 mmol, 2.45 ml, 1.5 eq) were mixed in dichloromethane (100 ml), after that di-tert-butyl dicarbonate (2.55 g, 11.71 mmol, 2.69 ml, 1 eq) was added at 0° C. The resulting mixture was stirred at room temperature overnight. After 15 hours the reaction mixture was washed with water (50 ml) and saturated aqueous solution of NaHCO3 (50 ml). The organic layer was separated, dried over anhydrous Na2SO4 and filtered. The filtrate collected was concentrated under reduced pressure to afford the desired tert-butyl N-(4,7,10,13-tetraoxaoctadec-1-yn-18-yl)carbamate (3.7 g, 85.0% purity, 8.42 mmol, 71.9% yield). The product obtained was used in further experiments without any additional purification.

Step 5: The synthesis of tert-butyl N-[1-(2-{[2-(pyridin-3-yl)quinazolin-4-yl]amino}-2,3-dihydro-1H-inden-5-yl)-4,7,10,13-tetraoxaoctadec-1-yn-18-yl]carbamate. The solution of tert-butyl N-(4,7,10,13-tetraoxaoctadec-1-yn-18-yl)carbamate (965.18 mg, 2.58 mmol) and N-(5-iodo-2,3-dihydro-1H-inden-2-yl)-2-(pyridin-3-yl)quinazolin-4-amine (800.0 mg, 1.72 mmol) in the mixture of THF (5 ml) and TEA (5 ml) was flushed with argon three times. Then, under inert atmosphere, [1,1′-bis(diphenylphosphino)ferrocene]dichloropalladium (II) dichloromethane adduct (281.39 mg, 344.57 μmol) and copper (I) iodide (65.62 mg, 344.57 μmol) were added to the reaction mixture, which was then heated up to 50° C. and left while stirring at this temperature overnight. After 18 hours the reaction mixture was concentrated under reduced pressure and the residue obtained was diluted with distilled water (100 ml) and extracted with ethyl acetate (3×50 ml). The organic layers were combined, washed with brine (50 ml), dried over anhydrous Na2SO4 and filtered. The filtrate collected was concentrated under reduced pressure to afford crude product, which was subjected for flash chromatography purification (Interchim, 40 g SiO2, MtBE/methanol with methanol from 0˜35%, flow rate=40 ml/min, Rf=7 CV) to result in the desired tert-butyl N-[1-(2-[2-(pyridin-3-yl)quinazolin-4-yl]amino-2,3-dihydro-1H-inden-5-yl)-4,7,10,13-tetraoxaoctadec-1-yn-18-yl]carbamate (450.0 mg, 85.0% purity, 538.83 μmol, 31.3% yield). LCMS (2 min, 95.1% by UV 215 nm, RT=1.501, (M+)=710.2).

Step 6: The synthesis of N-[5-(18-amino-4,7,10,13-tetraoxaoctadec-1-yn-1-yl)-2,3-dihydro-1H-inden-2-yl]-2-(pyridin-3-yl)quinazolin-4-amine. The starting tert-butyl N-[1-(2-[2-(pyridin-3-yl)quinazolin-4-yl]amino-2,3-dihydro-1H-inden-5-yl)-4,7,10,13-tetraoxaoctadec-1-yn-18-yl]carbamate (450.0 mg, 633.92 μmol) was dissolved in absolute methanol (25 ml) and 2.2M HCl in 1,4-dioxane (25 ml) was added to the resulting solution. The reaction mixture was left while stirring overnight at room temperature. After 12 hours the reaction mixture was concentrated under reduced pressure to afford the desired N-[5-(18-amino-4,7,10,13-tetraoxaoctadec-1-yn-1-yl)-2,3-dihydro-1H-inden-2-yl]-2-(pyridin-3-yl)quinazolin-4-amine (350.0 mg, 85.0% purity, 487.9 μmol, 76.9% yield); LCMS (86.1% by UV 215 nm, 2 min, RT=1.132, (M+)=610.4). The product obtained was used in further experiments without any additional purification.

Step 7: The synthesis of 3-{5-[2-oxo-4-(prop-2-enoyl)piperazin-1-yl]furan-2-yl}-N-[1-(2-{[2-(pyridin-3-yl)quinazolin-4-yl]amino}-2,3-dihydro-1H-inden-5-yl)-4,7,10,13-tetraoxaoctadec-1-yn-18-yl]propanamide (Z5431540269). To a solution of 3-5-[2-oxo-4-(prop-2-enoyl)piperazin-1-yl]furan-2-ylpropanoic acid (57.53 mg, 196.83 μmol) in DMF (10 ml) at 0° C. was added [(dimethylamino)(3H-[1,2,3]triazolo[4,5-b]pyridin-3-yloxy)methylidene]dimethylazanium hexafluorophosphanuide (99.79 mg, 262.43 μmol), ethylbis(propan-2-yl)amine (169.59 mg, 1.31 mmol, 230.0 μl, 10 eq) and after 5 min of stirring N-[5-(18-amino-4,7,10,13-tetraoxaoctadec-1-yn-1-yl)-2,3-dihydro-1H-inden-2-yl]-2-(pyridin-3-yl)quinazolin-4-amine (80.0 mg, 131.2 μmol). The resulting mixture was stirred at room temperature overnight. The completion of the reaction was monitored by LCMS. After that the reaction mixture solution was diluted with distilled water (40 ml) and extracted with DCM (3×25 ml). The organic layers were combined, washed with brine (3×10 ml), dried over anhydrous Na2SO4 and filtered. The filtrate collected was concentrated under reduced pressure. The crude product obtained was subjected for prep HPLC purification to result in the desired 3-5-[2-oxo-4-(prop-2-enoyl)piperazin-1-yl]furan-2-yl-N-[1-(2-[2-(pyridin-3-yl)quinazolin-4-yl]amino-2,3-dihydro-1H-inden-5-yl)-4,7,10,13-tetraoxaoctadec-1-yn-18-yl]propanamide (73.2 mg, 82.8 μmol, 63.1% yield) as yellow oil; LCMS (2 min, 100% by UV 215 nm, RT=1.297, (M+)=885).

### Example 18: Synthesis of N-[5-(18-azido-4,7,10,13-tetraoxaoctadec-1-yn-1-yl)-2,3-dihydro-1H-inden-2-yl]-2-(pyridin-3-yl)quinazolin-4-amine (Z5001897545)

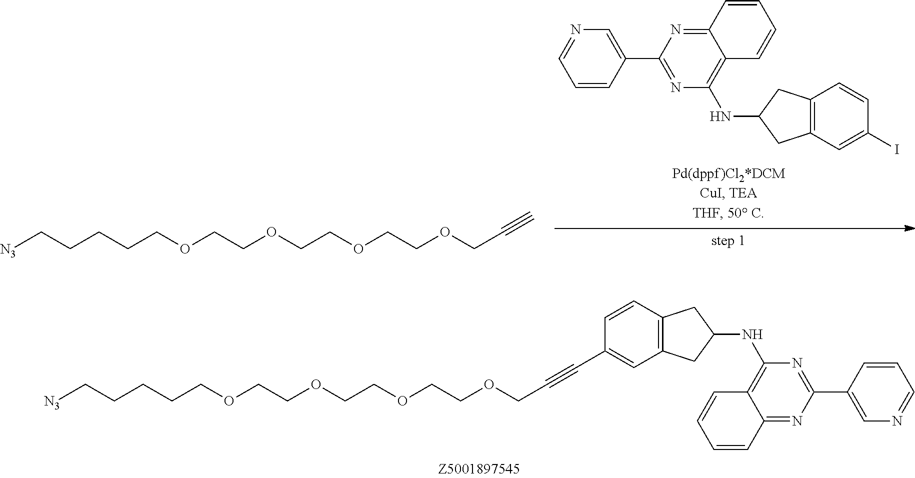 [SMILES: Ic1ccc2c(c1)CC(Nc1nc(-c3cccnc3)nc3ccccc13)C2 ; C#CCOCCOCCOCCOCCCCCN=[N+]=[N-] ; [N-]=[N+]=NCCCCCOCCOCCOCCOCC#Cc1ccc2c(c1)CC(Nc1nc(-c3cccnc3)nc3ccccc13)C2]

Step 1: Synthesis of N-[5-(18-azido-4,7,10,13-tetraoxaoctadec-1-yn-1-yl)-2,3-dihydro-1H-inden-2-yl]-2-(pyridin-3-yl)quinazolin-4-amine (Z5001897545). To a solution of 3-5-[2-oxo-4-(prop-2-enoyl)piperazin-1-yl]furan-2-ylpropanoic acid (57.53 mg, 196.83 μmol) in DMF (10 ml) at 0° C. was added [(dimethylamino)(3H-[1,2,3]triazolo[4,5-b]pyridin-3-yloxy)methylidene]dimethylazanium hexafluorophosphanuide (99.79 mg, 262.43 μmol), ethylbis(propan-2-yl)amine (169.59 mg, 1.31 mmol, 230.0 μl, 10 eq) and after 5 min of stirring N-[5-(18-amino-4,7,10,13-tetraoxaoctadec-1-yn-1-yl)-2,3-dihydro-1H-inden-2-yl]-2-(pyridin-3-yl)quinazolin-4-amine (80.0 mg, 131.2 μmol). The resulting mixture was stirred at room temperature overnight. The completion of the reaction was monitored by LCMS. After that the reaction mixture solution was diluted with distilled water (40 ml) and extracted with DCM (3×25 ml). The organic layers were combined, washed with brine (3×10 ml), dried over anhydrous Na2SO4 and filtered. The filtrate collected was concentrated under reduced pressure. The crude product obtained was subjected for prep HPLC purification to result in the desired 3-5-[2-oxo-4-(prop-2-enoyl)piperazin-1-yl]furan-2-yl-N-[1-(2-[2-(pyridin-3-yl)quinazolin-4-yl]amino-2,3-dihydro-1H-inden-5-yl)-4,7,10,13-tetraoxaoctadec-1-yn-18-yl]propanamide (73.2 mg, 82.8 μmol, 63.1% yield) as yellow oil; LCMS (2 min, 100% by UV 215 nm, RT=1.297, (M+)=885).

### Example 19: Synthesis of 3-{5-[2-oxo-4-(prop-2-enoyl)piperazin-1-yl]furan-2-yl}-N-(5-{[3-(2-{[2-(pyridin-3-yl)quinazolin-4-yl]amino}-2,3-dihydro-1H-inden-5-yl)prop-2-yn-1-yl]oxy}pentyl)propenamide (Z7019433551)

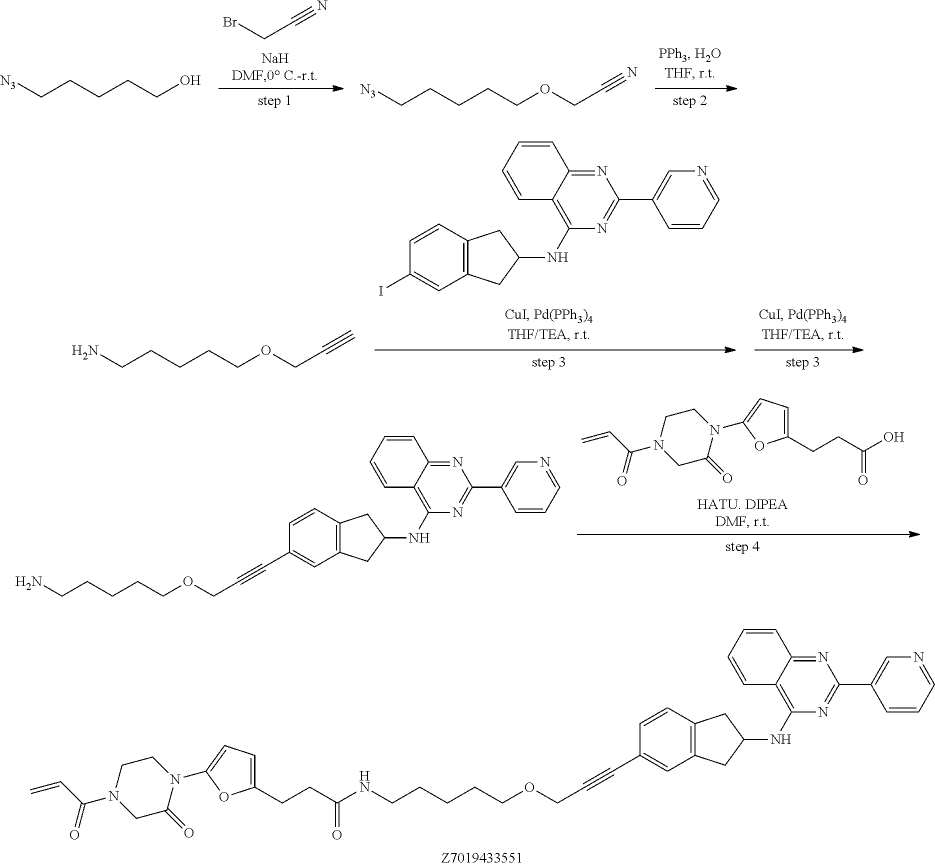

Step 1: The synthesis of 1-azido-5-(prop-2-yn-1-yloxy)pentane. The starting 5-azidopentan-1-ol (500.0 mg, 3.87 mmol) was dissolved in dry DMF (15 ml) and the resulting solution was cooled to 0° C. After that sodium hydride (309.52 mg, 12.9 mmol) was slowly added to the reaction mixture, followed by the addition of 3-bromoprop-1-yne (552.37 mg, 4.64 mmol, 350.0 μl, 1.2 equiv) via drops. The reaction mixture was then left while stirring at room temperature for 36 hours. After that period the mixture was diluted with distilled water (60 ml) and extracted with DCM (3×30 ml). The organic layers were combined, washed with water (3×25 ml), brine (20 ml), dried over anhydrous Na2SO4 and filtered. The filtrate collected was concentrated under reduced pressure to result in the desired 1-azido-5-(prop-2-yn-1-yloxy)pentane (603.0 mg, 90.0% purity, 3.25 mmol, 83.9% yield). The crude product obtained was of sufficient purity, so was used in further experiments without any additional purification.

Step 2: The synthesis of 5-(prop-2-yn-1-yloxy)pentan-1-amine. The starting 1-azido-5-(prop-2-yn-1-yloxy)pentane (602.71 mg, 3.6 mmol) was dissolved in THF (20 ml), after that triphenylphosphane (1.13 g, 4.33 mmol) was added to the resulting solution, followed by the addition of water (65.0 mg, 3.61 mmol, 70.0 μl, 1.0 eq). The reaction mixture was then left while stirring overnight at room temperature. After 12 hours the reaction mixture was concentrated under reduced pressure to result in the desired 5-(prop-2-yn-1-yloxy)pentan-1-amine (509.0 mg, 40.0% purity, 2.16 mmol, 60% yield). The crude obtained was used in further experiments without any additional purification.

Step 3: The synthesis of N-(5-{3-[(5-aminopentyl)oxy]prop-1-yn-1-yl}-2,3-dihydro-1H-inden-2-yl)-2-(pyridin-3-yl)quinazolin-4-amine. The starting N-(5-iodo-2,3-dihydro-1H-inden-2-yl)-2-(pyridin-3-yl)quinazolin-4-amine (209.01 mg, 450.17 μmol) was mixed together with 5-(prop-2-yn-1-yloxy)pentan-1-amine (70.0 mg, 495.71 μmol) and copper (I) iodide (8.57 mg, 45.02 μmol) in the mixture of dry THF (10 ml) and TEA (10 ml) under inert atmosphere (argon inlet). After that Pd(PPh3)4 (52.2 mg, 45.02 μmol) was added to the reaction mixture, which was then left while stirring overnight at room temperature. After 15 hours the reaction mixture was diluted with distilled water (30 ml) and extracted with DCM (3×20 ml). The organic layers were combined, washed with water (3×20 ml), brine (20 ml), dried over anhydrous Na2SO4 and filtered through a thin layer of silica. The filtrate collected was concentrated under reduced pressure and the crude residue obtained was subjected for prep HPLC purification to result in the desired N-(5-3-[(5-aminopentyl)oxy]prop-1-yn-1-yl-2,3-dihydro-1H-inden-2-yl)-2-(pyridin-3-yl)quinazolin-4-amine (50.0 mg, 52.34 μmol, 11.6% yield). LCMS (2 min, 100% by UV 215 nm, RT 0.869, (M+)=478.2).

Step 4: The synthesis of 3-{5-[2-oxo-4-(prop-2-enoyl)piperazin-1-yl]furan-2-yl}-N-(5-{[3-(2-{[2-(pyridin-3-yl)quinazolin-4-yl]amino}-2,3-dihydro-1H-inden-5-yl)prop-2-yn-1-yl]oxy}pentyl)propanamide (Z7019433551). The starting 3-5-[2-oxo-4-(prop-2-enoyl)piperazin-1-yl]furan-2-ylpropanoic acid (30.59 mg, 104.67 μmol) was dissolved in dry DMF (3 ml), after that [(dimethylamino)(3H-[1,2,3]triazolo[4,5-b]pyridin-3-yloxy)methylidene]dimethylazanium hexafluorophosphanuide (51.74 mg, 136.07 μmol) was added to the resulting solution, followed by the addition of ethylbis(propan-2-yl)amine (40.58 mg, 314.02 μmol, 50.0 μl, 3.0 eq). After 10 minutes N-(5-3-[(5-aminopentyl)oxy]prop-1-yn-1-yl-2,3-dihydro-1H-inden-2-yl)-2-(pyridin-3-yl)quinazolin-4-amine (50.0 mg, 104.69 μmol) was added to the reaction mixture, which was then left while stirring at room temperature overnight. After 12 hours the reaction mixture solution was subjected for prep HPLC purification without any further work-up (20-45% 0-5 min H2O/acetonitrile/0.1% FA, flow: 30 ml/min, column: Phenyl SMB100-5 100×19 mm) to result in the desired 3-5-[2-oxo-4-(prop-2-enoyl)piperazin-1-yl]furan-2-yl-N-(5-[3-(2-[2-(pyridin-3-yl)quinazolin-4-yl]amino-2,3-dihydro-1H-inden-5-yl)prop-2-yn-1-yl]oxy pentyl)propanamide (2.5 mg, 97.0% purity, 3.23 μmol, 3.1% yield) as white solid. LCMS (6 min, 97.43% by UV 215 nm, RT 3.458, (M+)=753.2).

### Example 20: Synthesis of 1-(3-{3-[3-methyl-4-({1-[1-(2-{[2-(pyridin-3-yl)quinazolin-4-yl]amino}-2,3-dihydro-1H-inden-5-yl)-4,7,10,13-tetraoxaoctadec-1-yn-18-yl]-1H-1,2,3-triazol-4-yl}methoxy)phenyl]-1-phenyl-1H-pyrazol-4-yl}prop-2-enoyl)piperidine-2-carboxamide (Z6559260025)

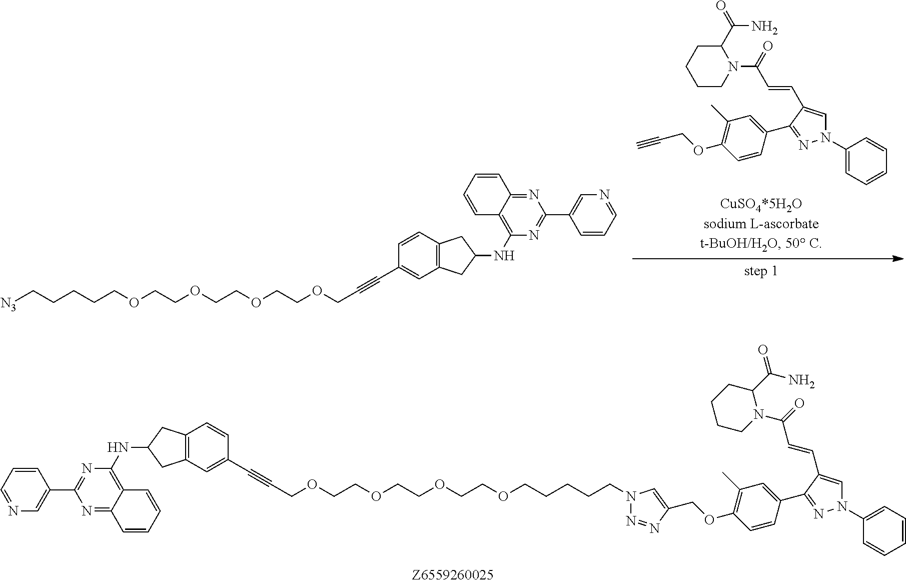 [SMILES: [N-]=[N+]=NCCCCCOCCOCCOCCOCC#Cc1ccc2c(c1)CC(Nc1nc(-c3cccnc3)nc3ccccc13)C2 ; C#CCOc1ccc(-c2nn(-c3ccccc3)cc2/C=C/C(=O)N2CCCCC2C(N)=O)cc1C ; Cc1cc(-c2nn(-c3ccccc3)cc2/C=C/C(=O)N2CCCCC2C(N)=O)ccc1OCc1cn(CCCCCOCCOCCOCCOCC#Cc2ccc3c(c2)CC(Nc2nc(-c4cccnc4)nc4ccccc24)C3)nn1]

Step 1: To a solution of 1-(3-3-[3-methyl-4-(prop-2-yn-1-yloxy)phenyl]-1-phenyl-1H-pyrazol-4-ylprop-2-enoyl)piperidine-2-carboxamide (11.07 mg, 23.63 μmol) in the mixture of t-BuOH (2 ml) and H2O (1 ml) at 0° C. was added sodium (2R)-2-[(1S)-1,2-dihydroxyethyl]-4-hydroxy-5-oxo-2,5-dihydrofuran-3-olate (7.02 mg, 35.45 μmol), followed by the addition of copper (II) sulfate pentahydrate (1.77 mg, 7.09 μmol) and the solution of N-[5-(18-azido-4,7,10,13-tetraoxaoctadec-1-yn-1-yl)-2,3-dihydro-1H-inden-2-yl]-2-(pyridin-3-yl)quinazolin-4-amine (15.0 mg, 23.59 μmol) in THF (1 ml). The resulting reaction mixture was then heated up to 50° C. and left while stirring overnight. After 16 hours the reaction mixture was concentrated under reduced pressure and the residue obtained was dissolved in DMSO. The resulting mixture was filtered through celite pad and the filtrate collected was subjected for prep HPLC purification without any further work-up (60-100% 0-5 min H2O/acetonitrile, flow: 30 ml/min; column: XBridge C18 OBD 100×19 mm, 5 um) to result in the desired 1-(3-3-[3-methyl-4-(1-[1-(2-[2-(pyridin-3-yl)quinazolin-4-yl]amino-2,3-dihydro-1H-inden-5-yl)-4,7,10,13-tetraoxaoctadec-1-yn-18-yl]-1H-1,2,3-triazol-4-ylmethoxy)phenyl]-1-phenyl-1H-pyrazol-4-ylprop-2-enoyl)piperidine-2-carboxamide (3.9 mg, 3.53 μmol, 14.9% yield). LCMS (4 min, 100% by UV 215 nm, RT 3.051, (M+)=1105.2).

### Example 21: Synthesis of N-[2-(3-{[4-(2-{[1-(2-{[2-(pyridin-3-yl)quinazolin-4-yl]amino}-2,3-dihydro-1H-inden-5-yl)-4,7,10,13-tetraoxaoctadec-1-yn-18-yl]carbamoyl}ethyl)piperazin-1-yl]methyl}imidazo[2,1-b][1,3]thiazol-6-yl)phenyl]quinoxaline-2-carboxamide (Z5787558140)

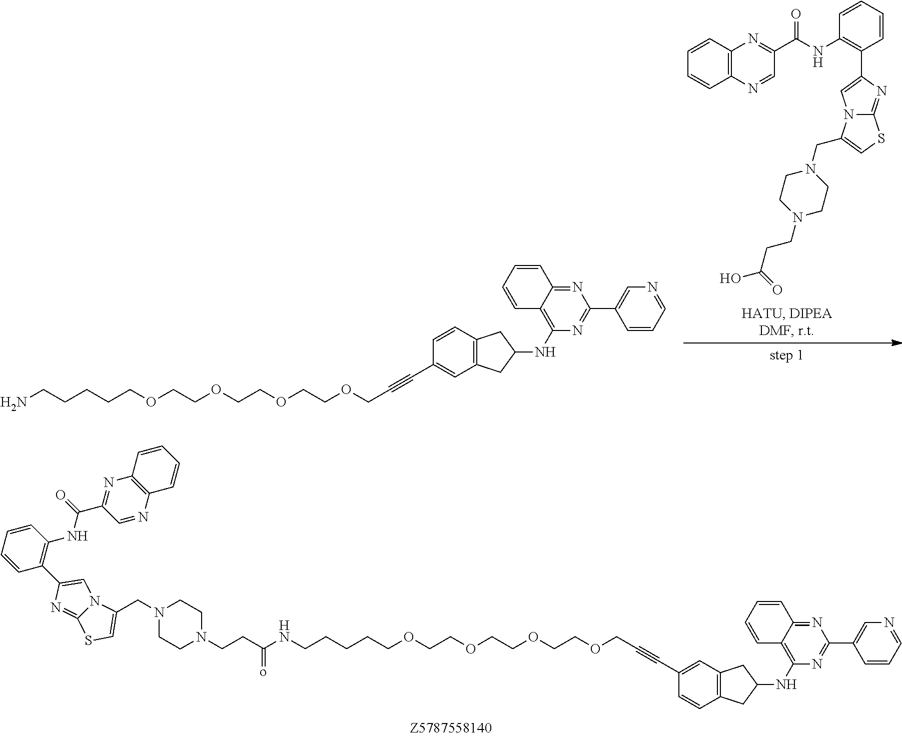

Step 1: To a solution of 3-[4-(6-[2-(quinoxaline-2-amido)phenyl]imidazo[2,1-b][1,3]thiazol-3-ylmethyl)piperazin-1-yl]propanoic acid (24.85 mg, 45.88 μmol) in dry DMF (2 mL) at 0° C. was added (1H-1,2,3-benzotriazol-1-yloxy)tris(dimethylamino)phosphonium hexafluorophosphanuide (26.38 mg, 59.65 μmol), followed by the addition of ethylbis(propan-2-yl)amine (29.65 mg, 229.4 μmol, 40.0 μl, 5.0 eq) and N-[5-(18-amino-4,7,10,13-tetraoxaoctadec-1-yn-1-yl)-2,3-dihydro-1H-inden-2-yl]-2-(pyridin-3-yl)quinazolin-4-amine (33.0 mg, 45.89 μmol). The resulting mixture was left while stirring at room temperature overnight. After 16 hours the reaction mixture solution was subjected for prep HPLC purification without any work-up (50-90% 0-5 min H2O/acetonitrile/NH4OH, flow: 30 ml/min; column: XBridge C18 OBD 100×19 mm, 5 um) to result in the desired N-[2-(3-[4-(2-[1-(2-[2-(pyridin-3-yl)quinazolin-4-yl]amino-2,3-dihydro-1H-inden-5-yl)-4,7,10,13-tetraoxaoctadec-1-yn-18-yl]carbamoylethyl)piperazin-1-yl]methylimidazo[2,1-b][1,3]thiazol-6-yl)phenyl]quinoxaline-2-carboxamide (10.2 mg, 9.0 μmol, 19.6% yield). LCMS (6 min, 100% by UV 215 nm, RT 3.800, (M+)=1133.4).

### Example 22: Synthesis of 4-(3-{3,5-diiodo-4-[3-(pyrrolidin-1-yl)propoxy]benzoyl}-1-benzofuran-2-yl)-N-[1-(2-{[2-(pyridin-3-yl)quinazolin-4-yl]amino}-2,3-dihydro-1H-inden-5-yl)-4,7,10,13-tetraoxaoctadec-1-yn-18-yl]butanamide (Z6618214640)

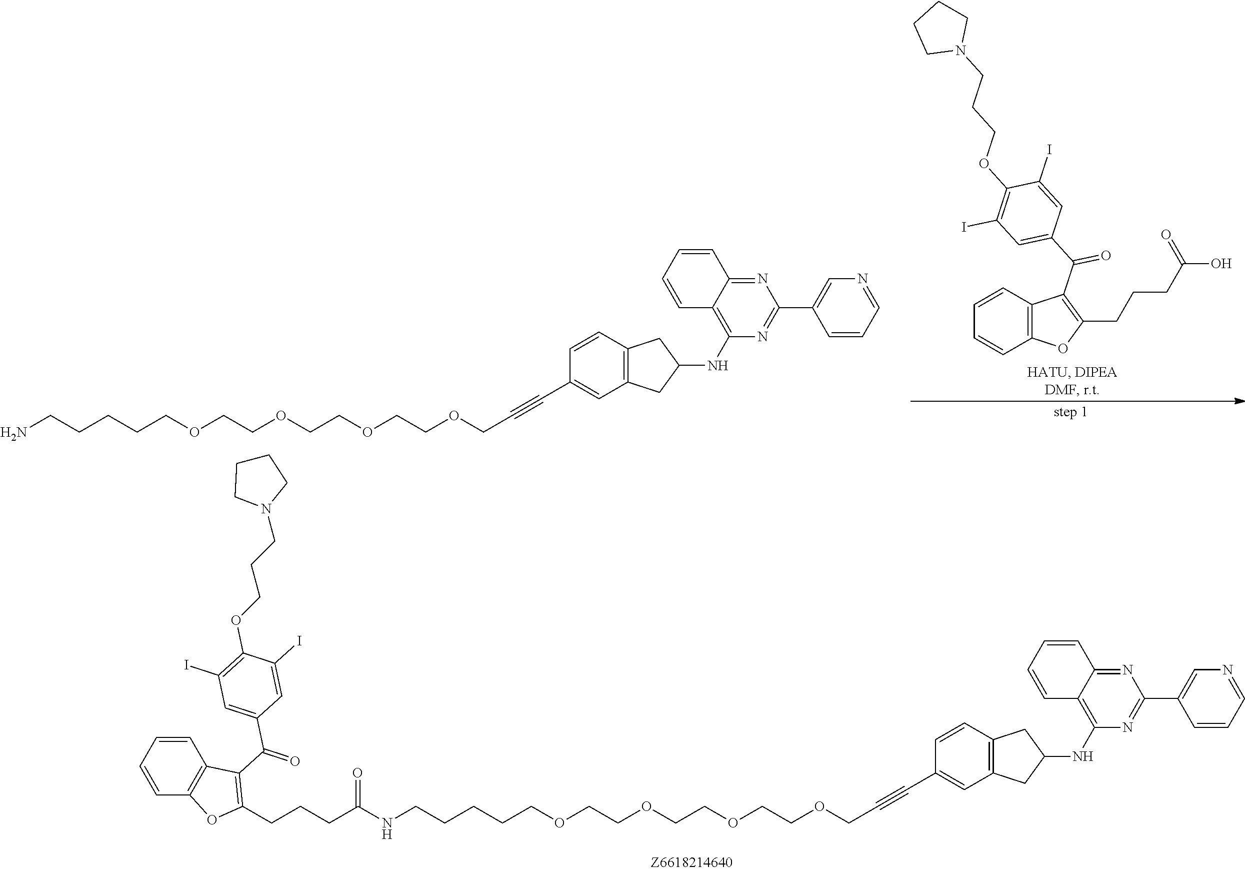

Step 1: To a solution of 4-(3-3,5-diiodo-4-[3-(pyrrolidin-1-yl)propoxy]benzoyl-1-benzofuran-2-yl)butanoic acid (28.8 mg, 41.91 μmol) in dry DMF (2 mL) at 0° C. was added [(dimethylamino)(3H-[1,2,3]triazolo[4,5-b]pyridin-3-yloxy)methylidene]dimethylazanium hexafluorophosphanuide (20.71 mg, 54.48 μmol), followed by the addition of ethylbis(propan-2-yl)amine (54.17 mg, 419.1 μmol, 70.0 μl, 10.0 eq) and N-[5-(18-amino-4,7,10,13-tetraoxaoctadec-1-yn-1-yl)-2,3-dihydro-1H-inden-2-yl]-2-(pyridin-3-yl)quinazolin-4-amine trihydrochloride (30.14 mg, 41.91 μmol). The resulting reaction mixture was stirred at room temperature overnight. After 15 hours the reaction mixture solution was subjected for prep HPLC purification without any work-up (70-70-100% 0-1-6 min H2O/methanol, flow: 30 ml/min; column Chromatorex 18 SMB100-ST 100×19 mm, 5 um) to result in the desired 4-(3-3,5-diiodo-4-[3-(pyrrolidin-1-yl)propoxy]benzoyl-1-benzofuran-2-yl)-N-[1-(2-[2-(pyridin-3-yl)quinazolin-4-yl]amino-2,3-dihydro-1H-inden-5-yl)-4,7,10,13-tetraoxaoctadec-1-yn-18-yl]butanamide (3.2 mg, 2.5 μmol, 6% yield) with the following spectra data: LCMS (2 min, 100% by UV 215 nm, RT 1.477, (M+)=1279.0).

### Example 23: Synthesis of 6-(2-{[2-(pyridin-3-yl)quinazolin-4-yl]amino}-2,3-dihydro-1H-inden-5-yl)hex-5-ynoic acid (Z6012191794)

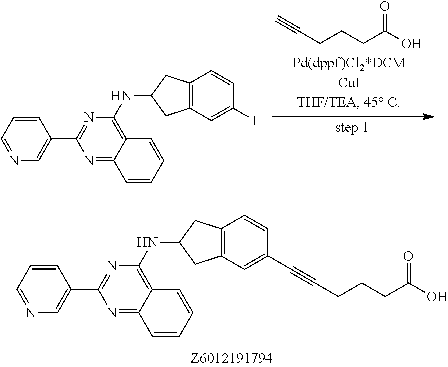

Step 1: The synthesis of 6-(2-{[2-(pyridin-3-yl)quinazolin-4-yl]amino}-2,3-dihydro-1H-inden-5-yl)hex-5-ynoic acid (Z6012191794). The starting N-(5-iodo-2,3-dihydro-1H-inden-2-yl)-2-(pyridin-3-yl)quinazolin-4-amine (100.0 mg, 215.38 μmol) and hex-5-ynoic acid (72.45 mg, 646.13 μmol) were mixed together in the mixture of THF (5 ml) and TEA (5 ml). The resulting solution was degassed and flushed with argon three times. After that [1,1′-bis(diphenylphosphino)ferrocene]dichloropalladium (II) dichloromethane adduct (35.18 mg, 43.08 μmol) was added to the reaction mixture under inert atmosphere, followed by the addition of copper (I) iodide (8.2 mg, 43.08 μmol). The reaction mixture was then stirred at 45° C. for 18 hours under inert atmosphere. After that period the mixture was concentrated under reduced pressure and the residue obtained was diluted with saturated aqueous solution of ammonia chloride (10 ml). The precipitate formed was collected by filtration, washed with diethyl ether (2×10 ml) and dried under reduced pressure. The product obtained was subjected for prep HPLC purification (20-40% 0-5 min H2O/ACN flow: 30 ml/min; column: XBridge C18 OBD 100×19 mm, 5 um) to result in the desired 6-(2-[2-(pyridin-3-yl)quinazolin-4-yl]amino-2,3-dihydro-1H-inden-5-yl)hex-5-ynoic acid (24.2 mg, 95.0% purity, 51.26 μmol, 23.8% yield) as beige solid. LCMS (2 min, 100% by UV 215 nm, RT 1.028, (M+)=449.2).

### Example 24: Synthesis of (2S,4R)-1-[(2S)-3,3-dimethyl-2-{14-[6-(2-{[2-(pyridin-3-yl)quinazolin-4-yl]amino}-2,3-dihydro-1H-inden-5-yl)hex-5-ynamido]-3,6,9,12-tetraoxatetradecanamido}butanoyl]-4-hydroxy-N-{[4-(4-methyl-1,3-thiazol-5-yl)phenyl]methyl}pyrrolidine-2-carboxamide (Z6308600108)

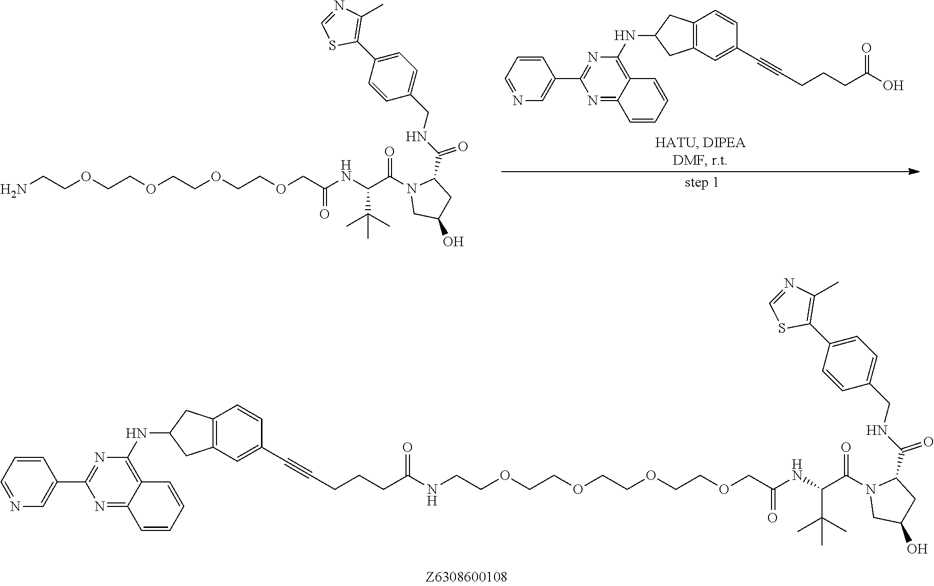 [SMILES: Cc1ncsc1-c1ccc(CNC(=O)[C@@H]2C[C@@H](O)CN2C(=O)[C@@H](NC(=O)COCCOCCOCCOCCN)C(C)(C)C)cc1 ; O=C(O)CCCC#Cc1ccc2c(c1)CC(Nc1nc(-c3cccnc3)nc3ccccc13)C2 ; Cc1ncsc1-c1ccc(CNC(=O)[C@@H]2C[C@@H](O)CN2C(=O)[C@@H](NC(=O)COCCOCCOCCOCCNC(=O)CCCC#Cc2ccc3c(c2)CC(Nc2nc(-c4cccnc4)nc4ccccc24)C3)C(C)(C)C)cc1]

Step 1: The synthesis of (2S,4R)-1-[(2S)-3,3-dimethyl-2-{14-[6-(2-{[2-(pyridin-3-yl)quinazolin-4-yl]amino}-2,3-dihydro-1H-inden-5-yl)hex-5-ynamido]-3,6,9,12-tetraoxatetradecanamido}butanoyl]-4-hydroxy-N-{[4-(4-methyl-1,3-thiazol-5-yl)phenyl]methyl}pyrrolidine-2-carboxamide (Z6308600108). The starting 6-(2-[2-(pyridin-3-yl)quinazolin-4-yl]amino-2,3-dihydro-1H-inden-5-yl)hex-5-ynoic acid (16.9 mg, 37.68 μmol) was dissolved in dry DMF (3 ml), after that [(dimethylamino)(3H-[1,2,3]triazolo[4,5-b]pyridin-3-yloxy)methylidene]dimethylazanium hexafluorophosphanuide (16.94 mg, 44.55 μmol) was added to the resulting solution, followed by the addition of ethylbis(propan-2-yl)amine (13.29 mg, 102.8 μmol). After 10 minutes (2S,4R)-1-[(2S)-2-(14-amino-3,6,9,12-tetraoxatetradecanamido)-3,3-dimethylbutanoyl]-4-hydroxy-N-[4-(4-methyl-1,3-thiazol-5-yl)phenyl]methylpyrrolidine-2-carboxamide (22.75 mg, 34.27 μmol) was added to the reaction mixture, which was then left while stirring at room temperature overnight. After 16 hours the reaction mixture solution was subjected for prep HPLC purification without any work-up (60-100% 0-5 min H₂O/methanol, flow: 30 ml/min; column: PHENYL SMB100-5 100×19 mm) to result in the desired (2S,4R)-1-[(2S)-3,3-dimethyl-2-14-[6-(2-[2-(pyridin-3-yl)quinazolin-4-yl]amino-2,3-dihydro-1H-inden-5-yl)hex-5-ynamido]-3,6,9,12-tetraoxatetradecanamidobutanoyl]-4-hydroxy-N-[4-(4-methyl-1,3-thiazol-5-yl)phenyl]methylpyrrolidine-2-carboxamide (5.3 mg, 4.84 μmol, 14.1% yield). LCMS (6 min, 100% by UV 215 nm, RT 3.411, (M+)=1095.4).

### Example 25: Synthesis of 2-(2-{[5-(3,5-dimethyl-1,2-oxazol-4-yl)-2-oxo-1,2-dihydropyridin-1-yl]methyl}phenoxy)acetic acid (Z7712308095)

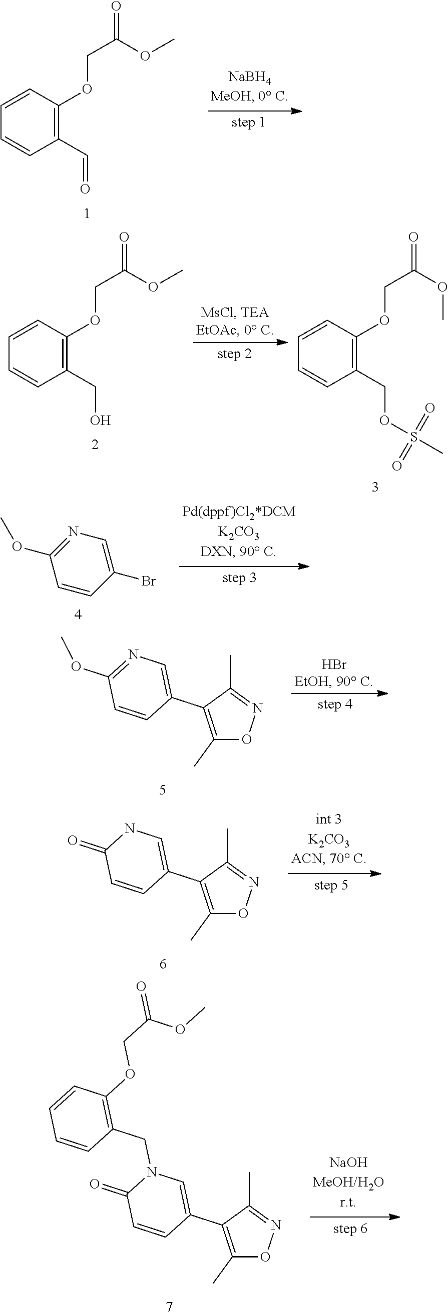

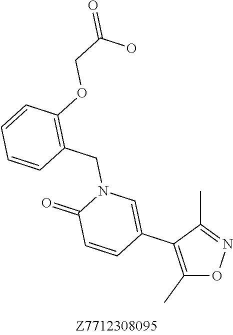 [SMILES: Cc1noc(C)c1-c1ccc(=O)n(Cc2ccccc2OCC(=O)O)c1]

Step 1: The synthesis of methyl 2-[2-(hydroxymethyl)phenoxy]acetate. To a solution of methyl 2-(2-formylphenoxy)acetate (1.0 g, 5.15 mmol) in MeOH (10 ml) sodium borohydride (194.75 mg, 5.12 mmol) was added portion wise at 0° C. Then the solution was stirred at room temperature for 2 hours, diluted with 10% HCl (20 ml), extracted with MtBE (2×15 ml), washed with brine (20 ml), dried over anhydrous sodium sulfate and filtered. The filtrate collected was concentrated under reduced pressure to obtain the desired methyl 2-[2-(hydroxymethyl)phenoxy]acetate (750 mg, 75% yield) as a colorless oil, which was used without further purification.

Step 2: The synthesis of methyl 2-{2-[(methanesulfonyloxy)methyl]phenoxy}acetate. To a solution of methyl 2-[2-(hydroxymethyl)phenoxy]acetate (700.0 mg, 3.57 mmol) and triethylamine (541.75 mg, 5.36 mmol, 750.0 μl, 1.5 eq) in ethyl acetate (10 ml) methanesulfonyl chloride (408.86 mg, 3.59 mmol, 280.0 μl, 1.0 eq) was added at 0° C. The mixture was stirred for 30 min at 0° C., then diluted with distilled water (20 ml). The organic layer was separated, washed with water (30 ml) and brine (30 ml), dried over magnesium sulfate, filtered, and concentrated in vacuo to afford the desired methyl 2-2-[(methanesulfonyloxy)methyl]phenoxyacetate (0.9 g, 90% yield), which was immediately used as crude in the next step.

Step 3: The synthesis of 5-(3,5-dimethyl-1,2-oxazol-4-yl)-2-methoxypyridine. A suspension of 5-bromo-2-methoxypyridine (6.0 g, 32.09 mmol), (dimethyl-1,2-oxazol-4-yl)boronic acid (5.85 g, 41.45 mmol), dipotassium carbonate (8.82 g, 63.96 mmol) and [1,1′-bis(diphenylphosphino)ferrocene]dichloropalladium (II) dichloromethane complex (1.3 g, 1.6 mmol) in a degassed mixture of dioxane and water (10:1, 100 ml) was stirred under argon at 90° C. overnight. After cooling to room temperature, the mixture was diluted with water (100 ml) and extracted with ethyl acetate (2×50 ml). The organic layers were combined, washed with water (100 ml) and brine (100 ml), dried over magnesium sulfate, filtered, and concentrated in vacuo to afford the desired 5-(3,5-dimethyl-1,2-oxazol-4-yl)-2-methoxypyridine (6 g, 92% yield) with the following spectra data: LCMS (2 min, 81.6% by UV 215 nm, RT 1.191, M+1=205.2).

Step 4: The synthesis of 5-(3,5-dimethyl-1,2-oxazol-4-yl)-1,2-dihydropyridin-2-one. To a magnetically stirred solution of 5-(3,5-dimethyl-1,2-oxazol-4-yl)-2-methoxypyridine (6.0 g, 29.4 mmol) in EtOH (20 ml) hydrogen bromide (82.83 g, 1.04 mol, 60.02 ml, 35 eq) was added. The resulting mixture was heated at 90° C. for 4 hours. Then the reaction mixture was cooled down to room temperature and concentrated under reduced pressure. The residue obtained was diluted with brine (50 ml) and the precipitate formed was collected by filtration and washed with small amount of water to result in the desired 5-(3,5-dimethyl-1,2-oxazol-4-yl)-1,2-dihydropyridin-2-one (4.5 g, 82% yield) with the following spectra data: LCMS (2 min, 100% by UV 215 nm, RT 0.596, M+1=191.0).

Step 5: The synthesis of methyl 2-(2-{[5-(3,5-dimethyl-1,2-oxazol-4-yl)-2-oxo-1,2-dihydropyridin-1-yl]methyl}phenoxy)acetate. A solution of 5-(dimethyl-1,2-oxazol-4-yl)pyridin-2-ol (356.77 mg, 1.88 mmol), methyl 2-2-[(methanesulfonyloxy)methyl]phenoxyacetate (900.0 mg, 3.28 mmol) and dipotassium carbonate (777.72 mg, 5.64 mmol) in acetonitrile (10 ml) was heated at 70° C. for 2 hours. Then the mixture was filtered, evaporated to dryness and the residue obtained was purified by flash chromatography (hexane:ethyl acetate=1:1) to obtain the desired methyl 2-(2-[5-(3,5-dimethyl-1,2-oxazol-4-yl)-2-oxo-1,2-dihydropyridin-1-yl]methylphenoxy)acetate (0.14 g, 20% yield) as white solid with the following spectra data: LCMS (2 min, 97.4% by UV 215 n, RT 1.123, M+1=369.0).

Step 6: The synthesis of 2-(2-{[5-(3,5-dimethyl-1,2-oxazol-4-yl)-2-oxo-1,2-dihydropyridin-1-yl]methyl}phenoxy)acetic acid (Z7712308095). To a solution of methyl 2-(2-[5-(3,5-dimethyl-1,2-oxazol-4-yl)-2-oxo-1,2-dihydropyridin-1-yl]methylphenoxy)acetate (200.0 mg, 543.28 μmol) in MeOH (5 ml) and water (1 ml) sodium hydroxide (65.01 mg, 1.63 mmol) was added at RT. The reaction mixture was stirred for 12 hours, then evaporated to dryness, dissolved in water (10 ml) and acidified to pH 2 with NaHSO₄. The precipitate formed was collected by filtration, washed with water and air-dried to result in the desired 2-(2-[5-(3,5-dimethyl-1,2-oxazol-4-yl)-2-oxo-1,2-dihydropyridin-1-yl]methylphenoxy)acetic acid (140 mg, 70% yield). 25 mg sample was purified by prep HPLC (20-20-50% 0-1-5 min H₂O/ACN/FA) to obtain 2-(2-[5-(3,5-dimethyl-1,2-oxazol-4-yl)-2-oxo-1,2-dihydropyridin-1-yl]methylphenoxy)acetic acid (8 mg) with the following spectra data: LCMS (6 min, 100% by UV 215 nm, RT 2.330, M+1=355.2).

### Example 26: Synthesis of Synthesis of 2-(3-{[5-(3,5-dimethyl-1,2-oxazol-4-yl)-2-oxo-1,2-dihydropyridin-1-yl]methyl}phenoxy)acetic acid (Z7712308083)

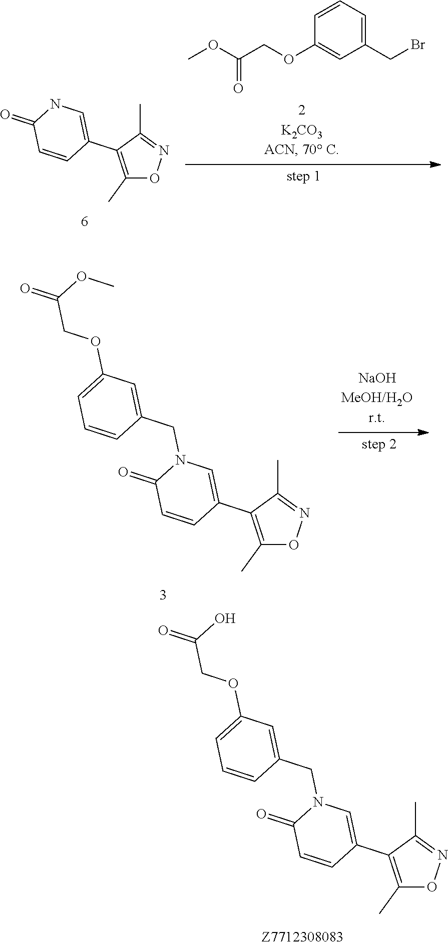 [SMILES: COC(=O)COc1cccc(CBr)c1 ; Cc1noc(C)c1-c1ccc(=O)[nH]c1 ; COC(=O)COc1cccc(Cn2cc(-c3c(C)noc3C)ccc2=O)c1 ; Cc1noc(C)c1-c1ccc(=O)n(Cc2cccc(OCC(=O)O)c2)c1]

Step 1: The synthesis of methyl 2-(3-{[5-(3,5-dimethyl-1,2-oxazol-4-yl)-2-oxo-1,2-dihydropyridin-1-yl]methyl}phenoxy)acetate. A mixture of 5-(dimethyl-1,2-oxazol-4-yl)pyridin-2-ol (1.0 g, 5.26 mmol), methyl 2-[3-(bromomethyl)phenoxy]acetate (1.36 g, 5.28 mmol) and dipotassium carbonate (1.45 g, 10.54 mmol) in acetonitrile (20 ml) was stirred at 70° C. for 2 hours. Then the reaction mixture was filtered, evaporated and purified by column chromatography (Interchim; 40 g SiO₂, hexane/ethyl acetate with ethyl acetate from 0˜95%, flow rate=40 ml/min, Rt=22 min) to result in the desired methyl 2-(3-[5-(3,5-dimethyl-1,2-oxazol-4-yl)-2-oxo-1,2-dihydropyridin-1-yl]methylphenoxy)acetate (750 mg, 38% yield) as yellow oil with the following spectra data: LCMS (2 min, 97.6% by UV 215 nm, RT 1.167, M+1=369.0).

Step 2: The synthesis of 2-(3-{[5-(3,5-dimethyl-1,2-oxazol-4-yl)-2-oxo-1,2-dihydropyridin-1-yl]methyl}phenoxy)acetic acid (Z7712308083). To a solution of methyl 2-(3-[5-(3,5-dimethyl-1,2-oxazol-4-yl)-2-oxo-1,2-dihydropyridin-1-yl]methylphenoxy)acetate (700.0 mg, 1.9 mmol) in MeOH (10 ml) and water (1 ml) sodium hydroxide (189.91 mg, 4.75 mmol) was added at RT. The reaction mixture was stirred for 12 hours, then evaporated to dryness and the residue obtained was dissolved in water (20 ml) and acidified to pH 2 with NaHSO4. The precipitate formed was collected by filtration, washed with water and air-dried to result in the desired 2-(3-[5-(3,5-dimethyl-1,2-oxazol-4-yl)-2-oxo-1,2-dihydropyridin-1-yl]methylphenoxy)acetic acid (470 mg, 95% purity, 68% yield). 25 mg sample was purified by prep HPLC (15-60% 0-5 min H2O/ACN/FA, column: XBridge OBD 100×30 mm, 5 um) to obtain pure 2-(3-[5-(3,5-dimethyl-1,2-oxazol-4-yl)-2-oxo-1,2-dihydropyridin-1-yl]methylphenoxy)acetic acid (18.9 mg) as beige solid with the following spectra data: LCMS (6 min, 100% by UV 215 nm, RT 2.588, M+1=355.2).

### Example 27: Synthesis of 2-iodo-1-(2,2,2-trifluoroethyl)-1H-indol-4-amine (Z7849369265)

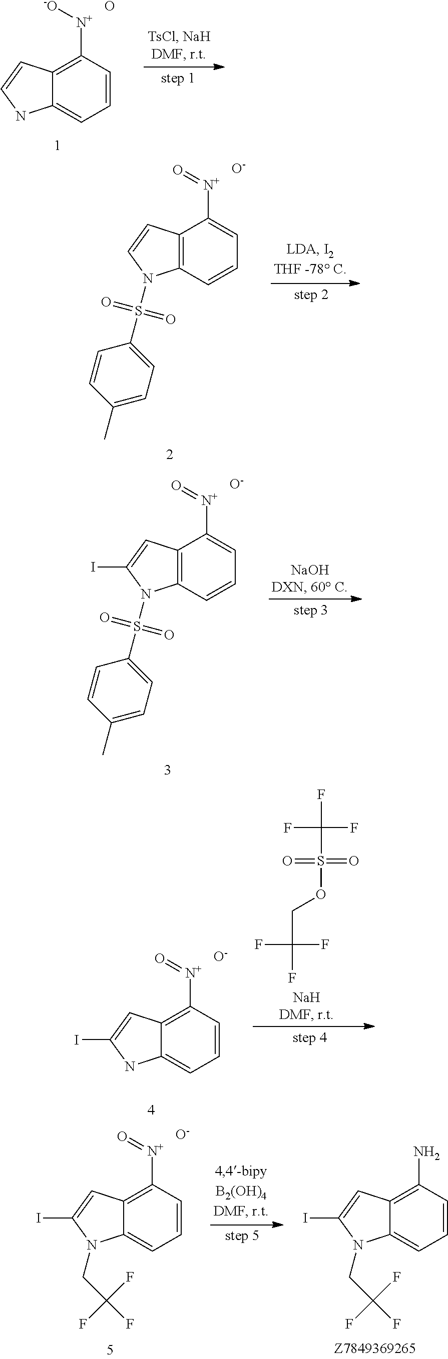

Step 1: The synthesis of 1-(4-methylbenzenesulfonyl)-4-nitro-1H-indole. To a solution of 4-nitro-1H-indole (50.0 g, 308.56 mmol) in anhydrous DMF (400 ml) under inert atmosphere was added sodium hydride (18.5 g, 770.92 mmol) in portions while stirring at 0° C. After the addition was completed, the resulting solution was allowed to warm up to room temperature and left while stirring at RT for 2 hours. After that period the reaction mixture was cooled down again and 4-methylbenzene-1-sulfonyl chloride (88.18 g, 464.16 mmol) was added in portions at 0° C. Then the mixture was stirred for 18 hours at RT. The reaction mixture was diluted with saturated solution of NH4Cl (2000 ml), stirred for 1 hour, then filtered. The precipitate collected was washed with water (3×500 ml), hexane (2×500 ml) and dried under reduced pressure to afford the desired 1-(4-methylbenzenesulfonyl)-4-nitro-1H-indole (95.0 g, 90.0% purity, 270.29 mmol, 87.7% yield).

Step 2: The synthesis of 2-iodo-1-(4-methylbenzenesulfonyl)-4-nitro-1H-indole. To bis(propan-2-yl)amine (5.12 g, 50.62 mmol, 7.16 ml, 3.2 eq) in dry THF (25 ml) at −78° C. was added lithium butan-1-ide (3.04 g, 47.46 mmol, 19.45 ml, 3.0 eq) and the mixture was stirred 10 minutes at −78° C. The resulting solution was added via cannula needle to the drip funnel and then dropwise to the solution of 1-(4-methylbenzenesulfonyl)-4-nitro-1H-indole (5.0 g, 15.82 mmol) in dry THF (50 ml) at −78° C. The mixture was left while stirring at the same temperature for 15 minutes. After that the solution of iodine (6.02 g, 23.73 mmol) in dry THF (25 ml) was added to the reaction mixture at −78° C. After the addition was completed the reaction mixture was allowed to warm to 0° C. Then the mixture was quenched with saturated aqueous solution of NH4Cl (200 ml) and diluted with ethyl acetate (200 ml). The organic layer was separated, washed with 10% aqueous solution of Na2S2O3 (150 ml), brine (150 ml), dried over anhydrous Na2SO4 and filtered. The filtrate collected was concentrated under reduced pressure and the crude residue obtained was subjected for flash chromatography purification (Interchim, 220 g SiO2, petroleum ether/EtOAc with EtOAc from 10˜45%, flow rate=100 ml/min, Rf=3.4-5 CV) to result in the desired 2-iodo-1-(4-methylbenzenesulfonyl)-4-nitro-1H-indole (1.53 g, 95.0% purity, 3.29 mmol, 20.8% yield) with the following spectra data: LCMS (2 min, 91.8% by UV 215 nm, RT 1.566, M+1=443.0); HNMR (purity 90%). The product obtained was of sufficient purity, so it was used in further experiments without any additional purification.

Step 3: The synthesis of 2-iodo-4-nitro-1H-indole. To a starting 2-iodo-1-(4-methylbenzenesulfonyl)-4-nitro-1H-indole (9.6 g, 21.72 mmol) in dioxane (250 ml) was added powdered sodium hydroxide (4.34 g, 108.56 mmol) and the reaction mixture was vigorously stirred at 60° C. overnight. The resulting suspension was then concentrated under reduced pressure, the residue obtained was diluted with water (150 ml) and extracted with ethyl acetate (3×100 ml). The organic layers were combined, dried over anhydrous Na2SO4 and filtered. The filtrate collected was concentrated under reduced pressure to afford the desired 2-iodo-4-nitro-1H-indole (5.0 g, 90.0% purity, 15.62 mmol, 72% yield) with the following spectra data: LCMS (2 min, 97.9% by UV 215 nm, RT 1.321, M+1=289.0).

Step 4: The synthesis of 2-iodo-4-nitro-1-(2,2,2-trifluoroethyl)-1H-indole. To a solution of 2-iodo-4-nitro-1H-indole (23.0 g, 79.88 mmol) in anhydrous DMF (250 ml) under inert atmosphere was added sodium hydride (4.15 g, 173.01 mmol) at 0° C. in portions. After the addition was completed, the resulting solution was allowed to warm up to room temperature and left while stirring at RT for 1 hour. After that period the reaction mixture was cooled down again and 2,2,2-trifluoroethyl trifluoromethanesulfonate (37.07 g, 159.8 mmol, 23.0 ml, 2.0 eq) was added dropwise at 0° C. Then the mixture was stirred for 18 hours at RT. The reaction mixture was diluted with saturated solution of NH4Cl (1000 ml), stirred for 1 hour, then filtered. The precipitate collected was washed with water (200 ml), hexane (2×200 ml) and dried under reduced pressure to afford the desired 2-iodo-4-nitro-1-(2,2,2-trifluoroethyl)-1H-indole (25.0 g, 90.0% purity, 60.8 mmol, 76.1% yield) with the following spectra data: LCMS (2 min, 92.5% by UV 215 nm, RT 1.290, M+1=370.8).

Step 5: The synthesis of 2-iodo-1-(2,2,2-trifluoroethyl)-1H-indol-4-amine (Z7849369265). To a solution of 2-iodo-4-nitro-1-(2,2,2-trifluoroethyl)-1H-indole (15.0 g, 40.55 mmol, 231.7 ml, 1.0 eq) and 4-(pyridin-4-yl)pyridine (316.54 mg, 2.03 mmol) in anhydrous DMF (250 ml) under inert atmosphere was added (dihydroxyboranyl)boronic acid (10.9 g, 121.09 mmol) at 5° C. in portions. The resulting reaction mixture solution was allowed to warm up to RT and stirred at room temperature for 1 hour. After that period the mixture was quenched by 10% aqueous solution of K2CO3 (1000 ml) and stirred for 1 hour. Then the mixture was extracted with ethyl acetate (3×200 ml). The organic layers were combined, washed with water (3×150 ml), brine (200 ml), dried over anhydrous Na2SO4 and filtered. The filtrate collected was concentrated under reduced pressure to result in the desired 2-iodo-1-(2,2,2-trifluoroethyl)-1H-indol-4-amine (14.0 g, 90.0% purity, 37.05 mmol, 91.4% yield) with the following spectra data: LCMS, BA080649-2 (2 min, 98.8% by UV 215 nm, RT 1.022, M+1=341.0); The product obtained was used in further experiments without any additional purification.

### Example 28: Synthesis of 2-(3-{[5-(3,5-dimethyl-1,2-oxazol-4-yl)-2-oxo-1,2-dihydropyridin-1-yl]methyl}phenoxy)-N-(2-{2-[2-(2-{4-[(3-{4-[(1-methylpiperidin-4-yl)amino]-1-(2,2,2-trifluoroethyl)-1H-indol-2-yl}prop-2-yn-1-yl)amino]phenoxy}ethoxy) ethoxy]ethoxy}ethyl)acetamide (Z7931922967)

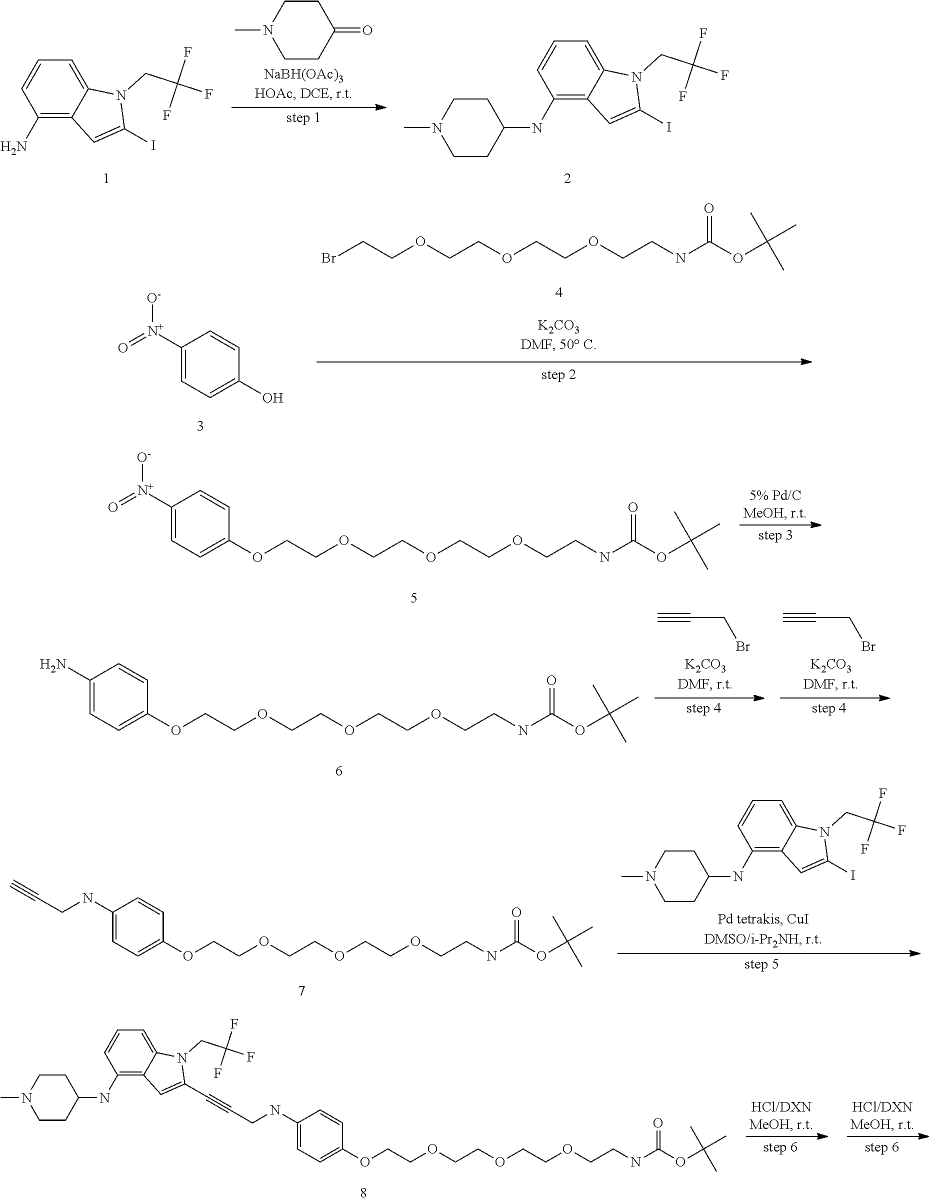

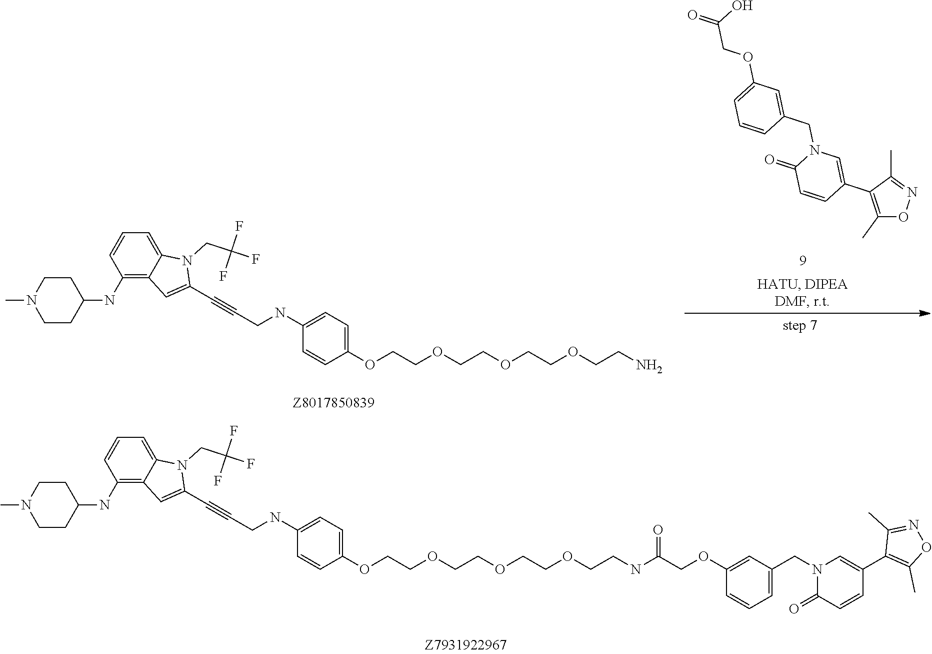 [SMILES: CN1CCC(Nc2cccc3c2cc(C#CCNc2ccc(OCCOCCOCCOCCN)cc2)n3CC(F)(F)F)CC1 ; Cc1noc(C)c1-c1ccc(=O)n(Cc2cccc(OCC(=O)O)c2)c1 ; Cc1noc(C)c1-c1ccc(=O)n(Cc2cccc(OCC(=O)NCCOCCOCCOCCOc3ccc(NCC#Cc4cc5c(NC6CCN(C)CC6)cccc5n4CC(F)(F)F)cc3)c2)c1]

Step 1: The synthesis of 2-iodo-N-(1-methylpiperidin-4-yl)-1-(2,2,2-trifluoroethyl)-1H-indol-4-amine. Sodium bis(acetyloxy)boranuidyl acetate (8.05 g, 37.94 mmol) was added to the solution of 2-iodo-1-(2,2,2-trifluoroethyl)-1H-indol-4-amine (4.3 g, 12.65 mmol), 1-methylpiperidin-4-one (2.0 g, 17.71 mmol) and acetic acid (2.28 g, 37.94 mmol, 2.19 ml, 3.0 eq in DCE (50 ml) at 0° C. in portions. The resulting mixture was left while stirring at room temperature overnight. After 18 hours sodium bis(acetyloxy)boranuidyl acetate (2.7 g, 12.73 mmol) and acetic acid (0.62 g, 10.40 mmol, 0.6 ml) were added and the reaction mixture was stirred at room temperature for another 18 hours. These additions were repeated until the reaction was completed (detected by LCMS). The saturated aqueous solution of K2CO3 (150 ml) was added carefully in portions to the reaction mixture, which was then extracted with DCM (3×100 ml). The organic layers were combined, washed with brine (150 ml), dried over anhydrous Na2SO4 and filtered. The filtrate collected was concentrated under reduced pressure and the crude residue obtained was treated with mixture of water/hexane (5/1, 80 ml) and the precipitate formed was collected by filtration, washed with water (15 ml) and air dried to afford the desired 2-iodo-N-(1-methylpiperidin-4-yl)-1-(2,2,2-trifluoroethyl)-1H-indol-4-amine (3.6 g, 85.0% purity, 7.0 mmol, 55.3% yield) as brown solid with the following spectra data: LCMS (2 min, 98.9% by UV 215 nm, RT 1.059, M+1=438.0). The product was used in further experiments without any additional purification.

Step 2: The synthesis of tert-butyl N-[2-(2-{2-[2-(4-nitrophenoxy)ethoxy]ethoxy}ethoxy)ethyl]carbamate. Dipotassium carbonate (2.91 g, 21.1 mmol) was added to a solution of 4-nitrophenol (2.34 g, 16.85 mmol) in DMF (40 ml). The resulting mixture was stirred for 30 minutes and after that tert-butyl N-(2-2-[2-(2-bromoethoxy)ethoxy]ethoxyethyl)carbamate (5.0 g, 14.08 mmol) was added in one portion to the reaction mixture, which was then heated up to 50° C. and left while stirring for 24 hours. After that period the resulting mixture was diluted with ethyl acetate (200 ml), washed with H2O (60 ml), brine (4×40 ml), dried over anhydrous Na2SO4 and filtered. The filtrate collected was concentrated under reduced pressure to afford the desired tert-butyl N-[2-(2-2-[2-(4-nitrophenoxy)ethoxy]ethoxyethoxy)ethyl]carbamate (4.3 g, 90.0% purity, 9.34 mmol, 66.5% yield) as yellow solid with the following spectra data: LCMS (2 min, 100% by UV 215 nm, RT 1.206, (M-Boc+1)=315.2).

Step 3: The synthesis of tert-butyl N-[2-(2-{2-[2-(4-aminophenoxy)ethoxy]ethoxy}ethoxy)ethyl]carbamate. To a solution of tert-butyl N-[2-(2-2-[2-(4-nitrophenoxy)ethoxy]ethoxyethoxy)ethyl]carbamate (3.8 g, 9.17 mmol) in methanol (70 ml) 5% Pd/C (0.38 g) was added. The resulting mixture was hydrogenated at 1 atm and ambient temperature overnight. After 15 hours the catalyst was filtered off and the filtrate collected was concentrated under reduced pressure to afford the desired tert-butyl N-[2-(2-2-[2-(4-aminophenoxy)ethoxy]ethoxyethoxy)ethyl]carbamate (3.5 g, 90.0% purity, 8.19 mmol, 89.4% yield) as yellow solid with the following spectra data: LCMS (2 min, 98.1% by UV 215 nm, RT 0.989, M+1=385.2). The product was used in further experiments without any additional purification.

Step 4: The synthesis of tert-butyl N-(2-{2-[2-(2-{4-[(prop-2-yn-1-yl)amino]phenoxy}ethoxy)ethoxy]ethoxy}ethyl)carbamate. 3-Bromoprop-1-yne (714.83 mg, 6.06 mmol, 460.0 μl, 1.05 eq) was added in portions to the solution of tert-butyl N-[2-(2-2-[2-(4-aminophenoxy)ethoxy]ethoxyethoxy)ethyl]carbamate (2.2 g, 5.73 mmol) and dipotassium carbonate (1.19 g, 8.6 mmol) in DMF (30 ml) and the mixture was stirred at RT for 24 hours. After that period the mixture was diluted with ethyl acetate (150 ml), washed with H2O (40 ml) and saturated aqueous solution of NaHSO3 (30 ml). The organic layer was separated, washed with brine (3×30 ml), dried over anhydrous Na2SO4 and filtered. The filtrate collected was concentrated under reduced pressure to afford 2.5 g of crude oil (app. 50% purity). The crude product obtained was subjected for flash chromatography purification (Interchim, 80 g SiO2, chloroform/acetonitrile with acetonitrile from 10˜95%, flow rate=60 ml/min, Rf=8-14 CV) to result in the desired tert-butyl N-(2-2-[2-(2-4-[(prop-2-yn-1-yl)amino]phenoxyethoxy)ethoxy]ethoxyethyl)carbamate (1.0 g, 90.0% purity, 2.13 mmol, 37.2% yield) as brown oil with the following spectra data: LCMS (2 min, 94.4% by UV 215 nm, RT 1.214, M+1=423.2).

Step 5: The synthesis of tert-butyl N-(2-{2-[2-(2-{4-[(3-{4-[(1-methylpiperidin-4-yl)amino]-1-(2,2,2-trifluoroethyl)-1H-indol-2-yl}prop-2-yn-1-yl)amino]phenoxy}ethoxy) ethoxy]ethoxy}ethyl)carbamate. Tetrakis(triphenylphosphine)palladium (158.9 mg, 137.19 μmol) was added to a flask charged with 2-iodo-N-(1-methylpiperidin-4-yl)-1-(2,2,2-trifluoroethyl)-1H-indol-4-amine (300.0 mg, 686.41 μmol), tert-butyl N-(2-2-[2-(2-4-[(prop-2-yn-1-yl)amino]phenoxyethoxy)ethoxy]ethoxyethyl)carbamate (347.56 mg, 823.13 μmol) and copper iodide (13.02 mg, 68.59 μmol) in DMSO/i-Pr2NH (3/1, 6 ml) at RT. The resulting mixture was bubbled with argon for 10 minutes, then left while stirring at RT for 12 hours. The mixture was diluted with ethyl acetate (50 ml), washed with H2O (15 ml), aqua NH3 (2×15 ml), brine (10 ml), dried over anhydrous Na2SO4 and filtered. The filtrate collected was concentrated under reduced pressure to afford 0.5 g of crude brown oil. The crude product obtained was purified by flash chromatography (Interchim; 40 g SiO2, MtBE/methanol with methanol from 0˜95%, flow rate=40 ml/min, Rf=13-19 CV) to result in the desired tert-butyl N-(2-2-[2-(2-4-[(3-4-[(1-methylpiperidin-4-yl)amino]-1-(2,2,2-trifluoroethyl)-1H-indol-2-ylprop-2-yn-1-yl)amino]phenoxyethoxy)ethoxy]ethoxyethyl)carbamate (300.0 mg, 90.0% purity, 368.93 μmol, 53.8% yield) as brown oil with the following spectra data: LCMS (2.5 min, 97.3% by UV 215 nm, RT 1.075, M+1=732.4).

Step 6: The synthesis of 2-(3-{[4-(2-{2-[2-(2-aminoethoxy)ethoxy]ethoxy}ethoxy) phenyl]amino}prop-1-yn-1-yl)-N-(1-methylpiperidin-4-yl)-1-(2,2,2-trifluoroethyl)-1H-indol-4-amine (Z8017950839, PE-P4-amine). The starting tert-butyl N-(2-2-[2-(2-4-[(3-4-[(1-methylpiperidin-4-yl)amino]-1-(2,2,2-trifluoroethyl)-1H-indol-2-ylprop-2-yn-1-yl)amino]phenoxyethoxy)ethoxy]ethoxyethyl)carbamate (480.0 mg, 655.88 μmol) was dissolved in absolute MeOH (15 ml). After that 10% HCl in dioxane (141.46 mg, 3.93 mmol, 1.97 ml, 6.0 eq) was added to the resulting solution, which was left while stirring at room temperature overnight. After 15 hours the mixture was concentrated under reduced pressure (<400C). The residue obtained was quenched with saturated aqueous solution of NaHCO3 (20 ml) and extracted with DCM (3×20 ml). The combined organic layers were washed with brine (10 ml), dried over anhydrous Na2SO4 and filtered. The filtrate collected was concentrated under reduced pressure to afford the desired 2-(3-[4-(2-2-[2-(2-aminoethoxy)ethoxy]ethoxyethoxy)phenyl]aminoprop-1-yn-1-yl)-N-(1-methylpiperidin-4-yl)-1-(2,2,2-trifluoroethyl)-1H-indol-4-amine (360.0 mg, 90.0% purity, 512.88 μmol, 78.3% yield) as brown oil with the following spectra data: LCMS (2.5 min, 100% by UV 215 nm, RT 1.011, M+1=632.2). The product was used in further experiments without any additional purification.

Step 7: The synthesis of 2-(3-{[5-(3,5-dimethyl-1,2-oxazol-4-yl)-2-oxo-1,2-dihydropyridin-1-yl]methyl}phenoxy)-N-(2-{2-[2-(2-{4-[(3-{4-[(1-methylpiperidin-4-yl)amino]-1-(2,2,2-trifluoroethyl)-1H-indol-2-yl}prop-2-yn-1-yl)amino]phenoxy}ethoxy)ethoxy]ethoxy}ethyl)acetamide (Z7931922967). (1H-1,2,3-benzotriazol-1-yloxy)tris(dimethylamino)phosphanium hexafluorophosphanuide (31.51 mg, 71.28 μmol) was added to the solution of 2-(3-[4-(2-2-[2-(2-aminoethoxy)ethoxy]ethoxyethoxy)phenyl]aminoprop-1-yn-1-yl)-N-(1-methylpiperidin-4-yl)-1-(2,2,2-trifluoroethyl)-1H-indol-4-amine (30.0 mg, 47.49 μmol), 2-(3-[5-(3,5-dimethyl-1,2-oxazol-4-yl)-2-oxo-1,2-dihydropyridin-1-yl]methylphenoxy)acetic acid (16.83 mg, 47.52 μmol) and ethylbis(propan-2-yl)amine (61.37 mg, 475.17 μmol, 80.0 μl, 10.0 eq) in DMF (1.5 ml). The reaction mixture was then left while stirring at RT overnight. After 12 hours the reaction mixture solution was subjected for prep HPLC purification without any further work-up (33-40-60-100% 0-2-7-7.1 min 30 ml/min water/ACN+NH3; column XBridge C18 19×100 mm, 5 um) to afford the desired 2-(3-[5-(3,5-dimethyl-1,2-oxazol-4-yl)-2-oxo-1,2-dihydropyridin-1-yl]methylphenoxy)-N-(2-2-[2-(2-4-[(3-4-[(1-methylpiperidin-4-yl)amino]-1-(2,2,2-trifluoroethyl)-1H-indol-2-ylprop-2-yn-1-yl)amino]phenoxyethoxy)ethoxy]ethoxyethyl)acetamide (17.8 mg, 97.0% purity, 17.84 μmol, 37.5% yield) as yellow oil with the following spectra data: LCMS (6 min, 99.04% by UV 215 nm, RT 3.197, M+1=968.4), HNMR, DMSO-d6 (H4556894—purity 95+%).

### Example 29: Synthesis of 3-methoxy-4-[(3-{4-[(1-methylpiperidin-4-yl)amino]-1-(2,2,2-trifluoroethyl)-1H-indol-2-yl}prop-2-yn-1-yl)amino]benzoic acid (Z7964537886) and N-(2-{2-[2-(2-aminoethoxy)ethoxy]ethoxy}ethyl)-3-methoxy-4-[(3-{4-[(1-methylpiperidin-4-yl)amino]-1-(2,2,2-trifluoroethyl)-1H-indol-2-yl}prop-2-yn-1-yl)amino]benzamide (Z8264783263)

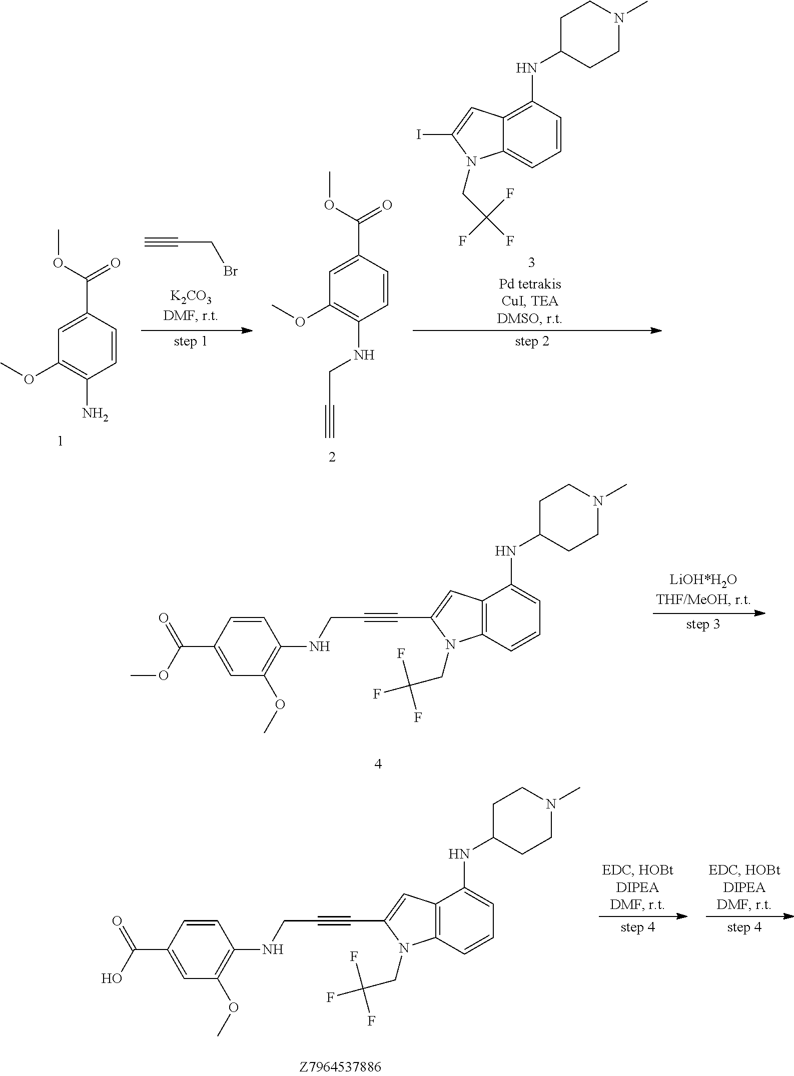

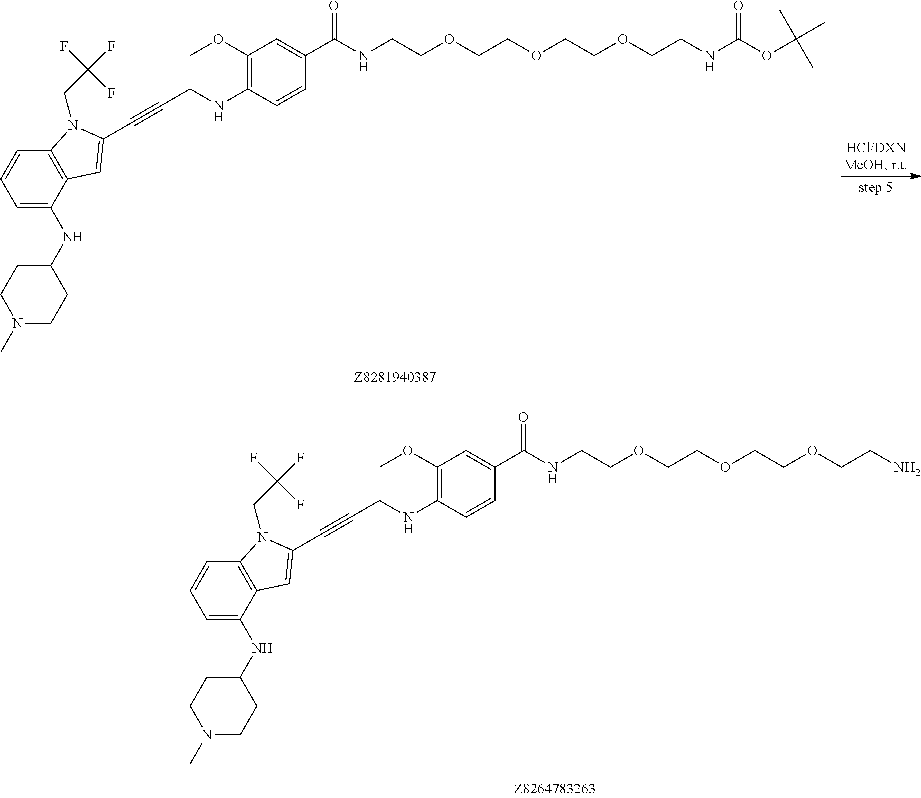 [SMILES: COc1cc(C(=O)NCCOCCOCCOCCNC(=O)OC(C)(C)C)ccc1NCC#Cc1cc2c(NC3CCN(C)CC3)cccc2n1CC(F)(F)F ; COc1cc(C(=O)NCCOCCOCCOCCN)ccc1NCC#Cc1cc2c(NC3CCN(C)CC3)cccc2n1CC(F)(F)F]

Step 1: The synthesis of methyl 3-methoxy-4-[(prop-2-yn-1-yl)amino]benzoate. To a solution of methyl 4-amino-3-methoxybenzoate (20.0 g, 110.45 mmol) in anhydrous DMF (110 ml) was added dipotassium carbonate (20.31 g, 147.27 mmol) and then under inert atmosphere 3-bromoprop-1-yne (9.65 g, 81.81 mmol, 6.15 ml, 1.0 eq) dropwise at 0° C. The resulting solution was then allowed to warm up to room temperature and left while stirring for 18 hours. The reaction mixture was diluted with saturated solution of NH4Cl (450 ml) and extracted with ethyl acetate (3×150 ml). The organic layers were combined, washed with water (3×100 ml), brine (100 ml), dried over anhydrous Na2SO4 and filtered. The filtrate collected was concentrated under reduced pressure and the crude obtained was subjected for flash chromatography (Interchim; 220 g SiO2, chloroform/acetonitrile with acetonitrile from 0˜25%, flow rate=60 ml/min, Rf=2-4.5 CV) to afford the desired methyl 3-methoxy-4-[(prop-2-yn-1-yl)amino]benzoate (7.0 g, 95.0% purity, 30.33 mmol, 41.2% yield) with the following spectra data: LCMS (2 min, 93.7% LCMS by UV 215 nm, RT 1.072, M+1=220.2).

Step 2: The synthesis of methyl 3-methoxy-4-[(3-{4-[(1-methylpiperidin-4-yl)amino]-1-(2,2,2-trifluoroethyl)-1H-indol-2-yl}prop-2-yn-1-yl)amino]benzoate. To a solution of 2-iodo-N-(1-methylpiperidin-4-yl)-1-(2,2,2-trifluoroethyl)-1H-indol-4-amine (2.0 g, 4.58 mmol), methyl 3-methoxy-4-[(prop-2-yn-1-yl)amino]benzoate (1.3 g, 5.95 mmol), copper (I) iodide (434.39 mg, 2.29 mmol) and triethylamine (1.39 g, 13.73 mmol, 1.94 ml, 3.0 eq) in DMSO (40 ml) under inert atmosphere triphenyl[tris(triphenyl-lambda5-phosphanyl)palladio]-lambda5-phosphane (530.1 mg, 457.65 μmol) was added. The resulting solution was stirred at RT for 18 hours. After that period the reaction mixture was diluted with distilled water (240 ml), the mixture was stirred for 1 hour. The precipitate formed was collected by filtration, washed with water (100 ml), dried and subjected for flash chromatography purification (Interchim; 120 g SiO2, MtBE/methanol (+2% TEA) with methanol from 0˜100%, flow rate=60 ml/min, Rf=7-9 CV) to afford the desired methyl 3-methoxy-4-[(3-4-[(1-methylpiperidin-4-yl)amino]-1-(2,2,2-trifluoroethyl)-1H-indol-2-ylprop-2-yn-1-yl)amino]benzoate (2.3 g, 90.0% purity, 3.92 mmol, 85.6% yield) with the following spectra data: LCMS (2 min, 96% by UV 215 nm, RT 1.003, M+1=529.2).

Step 3: The synthesis of 3-methoxy-4-[(3-{4-[(1-methylpiperidin-4-yl)amino]-1-(2,2,2-trifluoroethyl)-1H-indol-2-yl}prop-2-yn-1-yl)amino]benzoic acid (Z7964537886). To a solution of methyl 3-methoxy-4-[(3-4-[(1-methylpiperidin-4-yl)amino]-1-(2,2,2-trifluoroethyl)-1H-indol-2-ylprop-2-yn-1-yl)amino]benzoate (2.6 g, 4.92 mmol) in THF/MeOH (10/10 ml) the solution of lithium hydroxide hydrate (3.06 g, 72.8 mmol) in water (20 ml) was added. The resulting solution was stirred at room temperature for 48 hours. The reaction mixture was then concentrated under reduced pressure, diluted with water (20 ml) and acidified with sodium hydrogen sulfate (8.73 g, 72.8 mmol). The mixture was extracted with ethyl acetate/acetonitrile (3/2) (5×50 ml), dried over anhydrous Na2SO4 and filtered. The filtrate collected was concentrated under reduced pressure to afford the desired 3-methoxy-4-[(3-4-[(1-methylpiperidin-4-yl)amino]-1-(2,2,2-trifluoroethyl)-1H-indol-2-ylprop-2-yn-1-yl)amino]benzoic acid (1.8 g, 90.0% purity, 3.15 mmol, 64% yield) with the following spectra data: LCMS (2 min, 93.9% by UV 215 nm, RT 0.899, M+1=515.2).

Step 4: The synthesis of tert-butyl N-[2-(2-{2-[2-({3-methoxy-4-[(3-{4-[(1-methylpiperidin-4-yl)amino]-1-(2,2,2-trifluoroethyl)-1H-indol-2-yl}prop-2-yn-1-yl)amino]phenyl}formamido)ethoxy]ethoxy}ethoxy)ethyl]carbamate (Z8281940387). To a solution of 3-methoxy-4-[(3-4-[(1-methylpiperidin-4-yl)amino]-1-(2,2,2-trifluoroethyl)-1H-indol-2-ylprop-2-yn-1-yl)amino]benzoic acid (500.0 mg, 972.35 μmol) in DMF (10 ml) was added 3H-[1,2,3]triazolo[4,5-b]pyridin-3-ol (198.39 mg, 1.46 mmol), (3-[(ethylimino)methylidene]aminopropyl)dimethylamine hydrochloride (278.72 mg, 1.46 mmol), and after 5 min tert-butyl N-(2-2-[2-(2-aminoethoxy)ethoxy]ethoxyethyl)carbamate (312.5 mg, 1.07 mmol). The resulting mixture was stirred at RT overnight. The reaction mixture was diluted by water (40 ml), extracted with ethyl acetate (3×30 ml). The organic layers were combined, washed with water (3×20 ml), brine (50 ml), dried over anhydrous Na2SO4 and filtered. The filtrate collected was concentrated under reduced pressure and the residue obtained was subjected for prep HPLC purification (40-40-90% 0-1-5 min H2O/ACN/NH4OH flow: 60 ml/min, column: XBridge OBD C18 100×30 mm, 5 um) to result in the desired tert-butyl N-[2-(2-2-[2-(3-methoxy-4-[(3-4-[(1-methylpiperidin-4-yl)amino]-1-(2,2,2-trifluoroethyl)-1H-indol-2-ylprop-2-yn-1-yl)amino]phenylformamido)ethoxy]ethoxyethoxy)ethyl]carbamate (232.8 mg, 295.1 μmol, 30.4% yield) with the following spectra data: LCMS (6 min, 100% by UV 215 nm, RT 2.579, M+1=789.6).

Step 5: The synthesis of N-(2-{2-[2-(2-aminoethoxy)ethoxy]ethoxy}ethyl)-3-methoxy-4-[(3-{4-[(1-methylpiperidin-4-yl)amino]-1-(2,2,2-trifluoroethyl)-1H-indol-2-yl}prop-2-yn-1-yl)amino]benzamide (Z8264783263). To solution of tert-butyl N-[2-(2-2-[2-(3-methoxy-4-[(3-4-[(1-methylpiperidin-4-yl)amino]-1-(2,2,2-trifluoroethyl)-1H-indol-2-ylprop-2-yn-1-yl)amino]phenylformamido)ethoxy]ethoxyethoxy)ethyl]carbamate (232.0 mg, 294.08 μmol) in MeOH (2 ml) 10% HCl in dioxane (4 ml) was added. The reaction mixture was stirred at RT 18 hours. After that period the mixture was concentrated under reduced pressure to afford the desired N-(2-2-[2-(2-aminoethoxy)ethoxy]ethoxyethyl)-3-methoxy-4-[(3-4-[(1-methylpiperidin-4-yl)amino]-1-(2,2,2-trifluoroethyl)-1H-indol-2-ylprop-2-yn-1-yl)amino]benzamide (200.0 mg, 80.0% purity, 232.29 μmol, 78.8% yield) with the following spectra data: LCMS (2.5 min, 83.1% by UV 215 nm, RT 0.793, M+1=689.6). The crude scaffold obtained was used in further experiments without any additional purification.

### Example 30: Synthesis of 2-[(9S)-7-(4-chlorophenyl)-4,5,13-trimethyl-3-thia-1,8,11,12-tetraazatricyclo[8.3.0.0,2,6]trideca-2(6),4,7,10,12-pentaen-9-yl]-N-[2-(2-{2-[2-({3-methoxy-4-[(3-{4-[(1-methylpiperidin-4-yl)amino]-1-(2,2,2-trifluoroethyl)-1H-indol-2-yl}prop-2-yn-1-yl)amino]phenyl}formamido)ethoxy]ethoxy}ethoxy)ethyl]acetamide (Z8138894862)

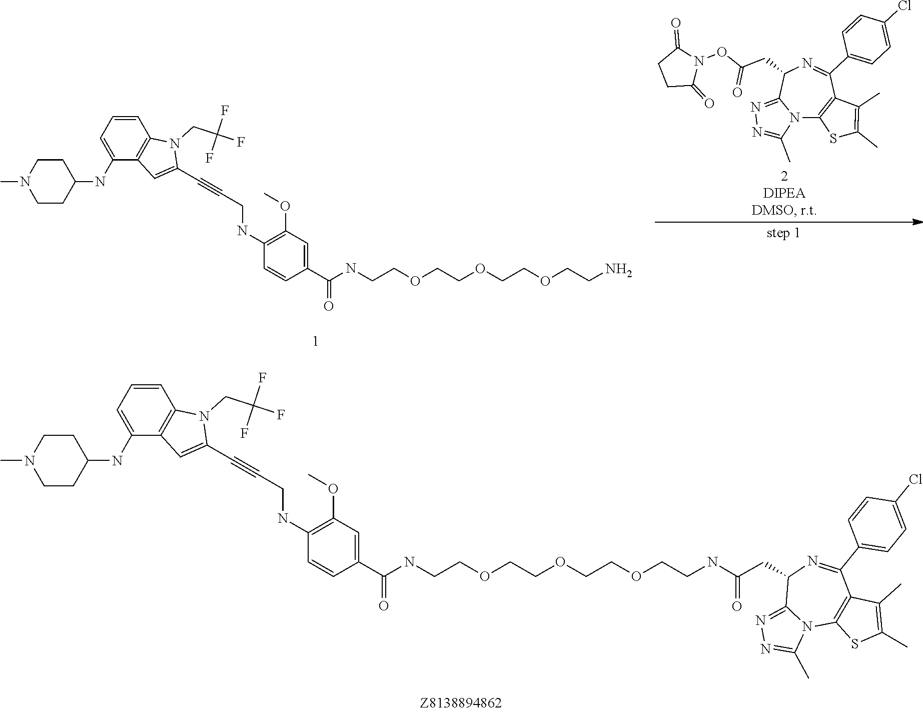 [SMILES: COc1cc(C(=O)NCCOCCOCCOCCN)ccc1NCC#Cc1cc2c(NC3CCN(C)CC3)cccc2n1CC(F)(F)F ; Cc1sc2c(c1C)C(c1ccc(Cl)cc1)=N[C@@H](CC(=O)ON1C(=O)CCC1=O)c1nnc(C)n1-2 ; COc1cc(C(=O)NCCOCCOCCOCCNC(=O)C[C@@H]2N=C(c3ccc(Cl)cc3)c3c(sc(C)c3C)-n3c(C)nnc32)ccc1NCC#Cc1cc2c(NC3CCN(C)CC3)cccc2n1CC(F)(F)F]

Step 1: The synthesis of 2-[(9S)-7-(4-chlorophenyl)-4,5,13-trimethyl-3-thia-1,8,11,12-tetraazatricyclo[8.3.0.0,2,6]trideca-2(6),4,7,10,12-pentaen-9-yl]-N-[2-(2-{2-[2-({3-methoxy-4-[(3-{4-[(1-methylpiperidin-4-yl)amino]-1-(2,2,2-trifluoroethyl)-1H-indol-2-yl}prop-2-yn-1-yl)amino]phenyl}formamido)ethoxy]ethoxy}ethoxy)ethyl]acetamide. The starting N-(2-2-[2-(2-aminoethoxy)ethoxy]ethoxyethyl)-3-methoxy-4-[(3-4-[(1-methylpiperidin-4-yl)amino]-1-(2,2,2-trifluoroethyl)-1H-indol-2-ylprop-2-yn-1-yl)amino]benzamide* (53.35 mg, 77.45 μmol), 2,5-dioxopyrrolidin-1-yl 2-[(9S)-7-(4-chlorophenyl)-4,5,13-trimethyl-3-thia-1,8,11,12-tetraazatricyclo[8.3.0.0,2,6]trideca-2(6),4,7,10,12-pentaen-9-yl]acetate (70.0 mg, 140.82 μmol) and ethylbis(propan-2-yl)amine (50.01 mg, 387.25 μmol, 70.0 μl, 5.0 equiv) were mixed in DMSO (2 ml) and left while stirring at RT for 18 hours. After that period the reaction mixture solution was subjected for prep HPLC without any further work-up (50-90% 0-6 min H2O/MeOH/NH4OH, flow: 60 ml/min, column: XBridge OBD C18 100×30 mm, 5 um) to afford the desired 2-[(9S)-7-(4-chlorophenyl)-4,5,13-trimethyl-3-thia-1,8,11,12-tetraazatricyclo[8.3.0.0,2,6]trideca-2(6),4,7,10,12-pentaen-9-yl]-N-[2-(2-2-[2-(3-methoxy-4-[(3-4-[(1-methylpiperidin-4-yl)amino]-1-(2,2,2-trifluoroethyl)-1H-indol-2-ylprop-2-yn-1-yl)amino]phenylformamido)ethoxy]ethoxyethoxy)ethyl]acetamide (5.0 mg, 4.67 μmol, 6% yield) as white solid with the following spectra data: LCMS (6 min, 100% by UV 215 nm, RT 3.055, M+1=1072.4).

### Example 31: The Synthesis of 2-(3-{[5-(3,5-dimethyl-1,2-oxazol-4-yl)-2-oxo-1,2-dihydropyridin-1-yl]methyl}phenoxy)-N-[2-(2-{2-[2-({3-methoxy-4-[(3-{4-[(1-methylpiperidin-4-yl)amino]-1-(2,2,2-trifluoroethyl)-1H-indol-2-yl}prop-2-yn-1-yl)amino]phenyl}formamido)ethoxy]ethoxy}ethoxy)ethyl]acetamide (Z8309636280)

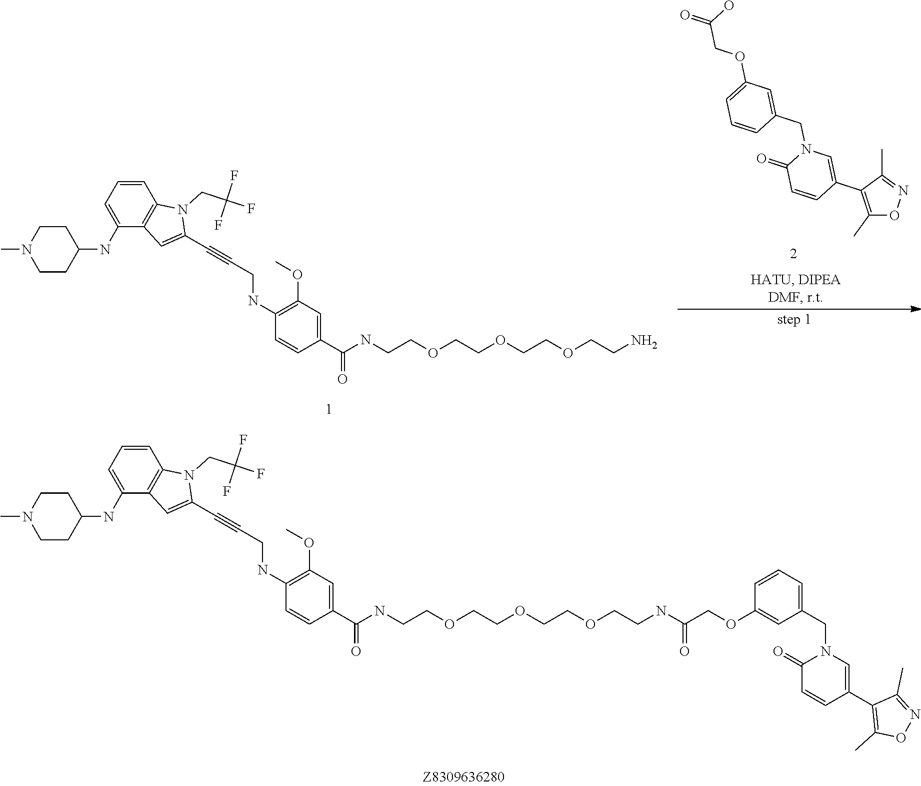 [SMILES: COc1cc(C(=O)NCCOCCOCCOCCN)ccc1NCC#Cc1cc2c(NC3CCN(C)CC3)cccc2n1CC(F)(F)F ; Cc1noc(C)c1-c1ccc(=O)n(Cc2cccc(OCC(=O)O)c2)c1 ; COc1cc(C(=O)NCCOCCOCCOCCNC(=O)COc2cccc(Cn3cc(-c4c(C)noc4C)ccc3=O)c2)ccc1NCC#Cc1cc2c(NC3CCN(C)CC3)cccc2n1CC(F)(F)F]

Step 1: The synthesis of 2-(3-{[5-(3,5-dimethyl-1,2-oxazol-4-yl)-2-oxo-1,2-dihydropyridin-1-yl]methyl}phenoxy)-N-[2-(2-{2-[2-({3-methoxy-4-[(3-{4-[(1-methylpiperidin-4-yl)amino]-1-(2,2,2-trifluoroethyl)-1H-indol-2-yl}prop-2-yn-1-yl)amino]phenyl}formamido)ethoxy]ethoxy}ethoxy)ethyl]acetamide (Z8309636280). The starting N-(2-2-[2-(2-aminoethoxy)ethoxy]ethoxyethyl)-3-methoxy-4-[(3-4-[(1-methylpiperidin-4-yl)amino]-1-(2,2,2-trifluoroethyl)-1H-indol-2-ylprop-2-yn-1-yl)amino]benzamide* (40.38 mg, 58.63 μmol), 2-(3-[5-(3,5-dimethyl-1,2-oxazol-4-yl)-2-oxo-1,2-dihydropyridin-1-yl]methylphenoxy)acetic acid** (20.76 mg, 58.63 μmol) [(dimethylamino)(3H-[1,2,3]triazolo[4,5-b]pyridin-3-yloxy)methylidene]dimethylazanium hexafluorophosphanuide (28.97 mg, 76.22 μmol) and ethylbis(propan-2-yl)amine (37.84 mg, 293.0 μmol, 50.0 μl, 5.0 eq) were mixed together in DMF (2 ml) and the mixture was left while stirring at RT overnight. After 12 hours the reaction mixture solution was subjected for prep HPLC purification without any further work-up (40-40-70% 0-1-6 min H2O/ACN/NH4OH flow: 60 ml/min, column: XBridge OBD C18 100×30 mm, 5 um) to afford the desired 2-(3-[5-(3,5-dimethyl-1,2-oxazol-4-yl)-2-oxo-1,2-dihydropyridin-1-yl]methylphenoxy)-N-[2-(2-2-[2-(3-methoxy-4-[(3-4-[(1-methylpiperidin-4-yl)amino]-1-(2,2,2-trifluoroethyl)-1H-indol-2-ylprop-2-yn-1-yl)amino]phenylformamido)ethoxy]ethoxyethoxy)ethyl]acetamide (6.7 mg, 97.0% purity, 6.34 μmol, 10.8% yield) with the following spectra data: LCMS, BA807178$1 (5 min, 97.09% by UV 215 n, RT 2.782, M+1=1025.4); HNMR, DMSO-d6 (H4518769—purity 95+%).

### Example 32: Synthesis of tert-butyl N-(5,8,11-trioxa-2-azatridecan-13-yl)carbamate (Z3379623823)

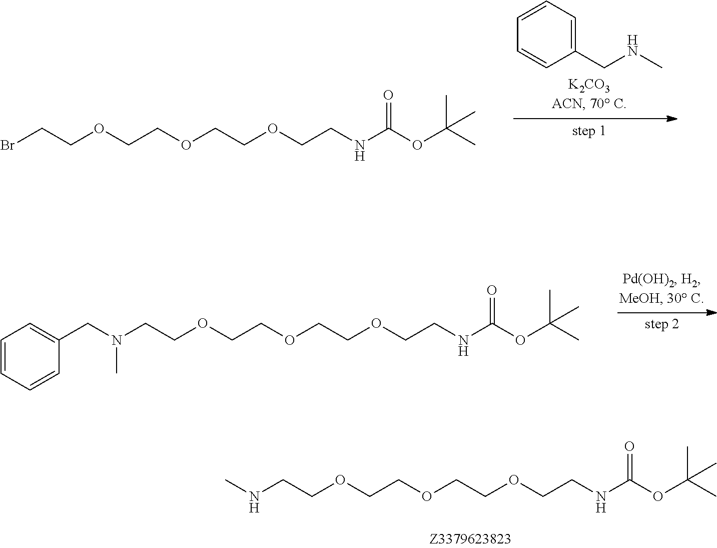

Step 1: The synthesis of tert-butyl N-(2-methyl-1-phenyl-5,8,11-trioxa-2-azatridecan-13-yl)carbamate. To a solution of tert-butyl N-(2-2-[2-(2-bromoethoxy)ethoxy]ethoxyethyl)carbamate (9.0 g, 25.35 mmol) in ACN (50.00 mL) was added benzyl(methyl)amine (6.14 g, 50.69 mmol) and dipotassium carbonate (10.49 g, 76.04 mmol). Then the reaction was stirred at 70° C. overnight. The suspension was filtered and the filtrate was purified by silica gel column chromatography (Interchim; 330 g SiO2, chloroform/ACN(50%)/methanol with methanol from 0˜10%, flow rate=100 mL/min) to afford tert-butyl N-(2-methyl-1-phenyl-5,8,11-trioxa-2-azatridecan-13-yl)carbamate (7.2 g, 71.6% yield) as a light yellow oil.

Step 2: The synthesis of tert-butyl N-(5,8,11-trioxa-2-azatridecan-13-yl)carbamate. To a solution of tert-butyl N-(2-methyl-1-phenyl-5,8,11-trioxa-2-azatridecan-13-yl)carbamate (3.0 g, 7.57 mmol) was added palladiumdiol (105.85 mg, 756.53 μmol) under N2 atmosphere. The suspension was degassed and purged with H2 for 3 times. The mixture was stirred under a hydrogen atmosphere (1 atm) at 30° C. for 8 h. The reaction mixture was filtered and concentrated under reduced pressure to give crude compound tert-butyl N-(5,8,11-trioxa-2-azatridecan-13-yl)carbamate (1.9 g) as a colorless oil. The crude product was used directly for the next step.

### Example 33: Synthesis of 2-(3-{[4-amino-6-(3,5-dimethyl-1,2-oxazol-4-yl)-1H-1,3-benzodiazol-1-yl]methyl}phenoxy)-N-(2-{2-[2-(2-[N-methyl-3-methoxy-4-[(3-[4-[(1-methylpiperidin-4-yl)amino]-1-(2,2,2-trifluoroethyl)-1H-indol-2-yl}prop-2-yn-1-yl)amino]benzenesulfonamido}ethoxy)ethoxy]ethoxy}ethyl)acetamide (Z8695255635)

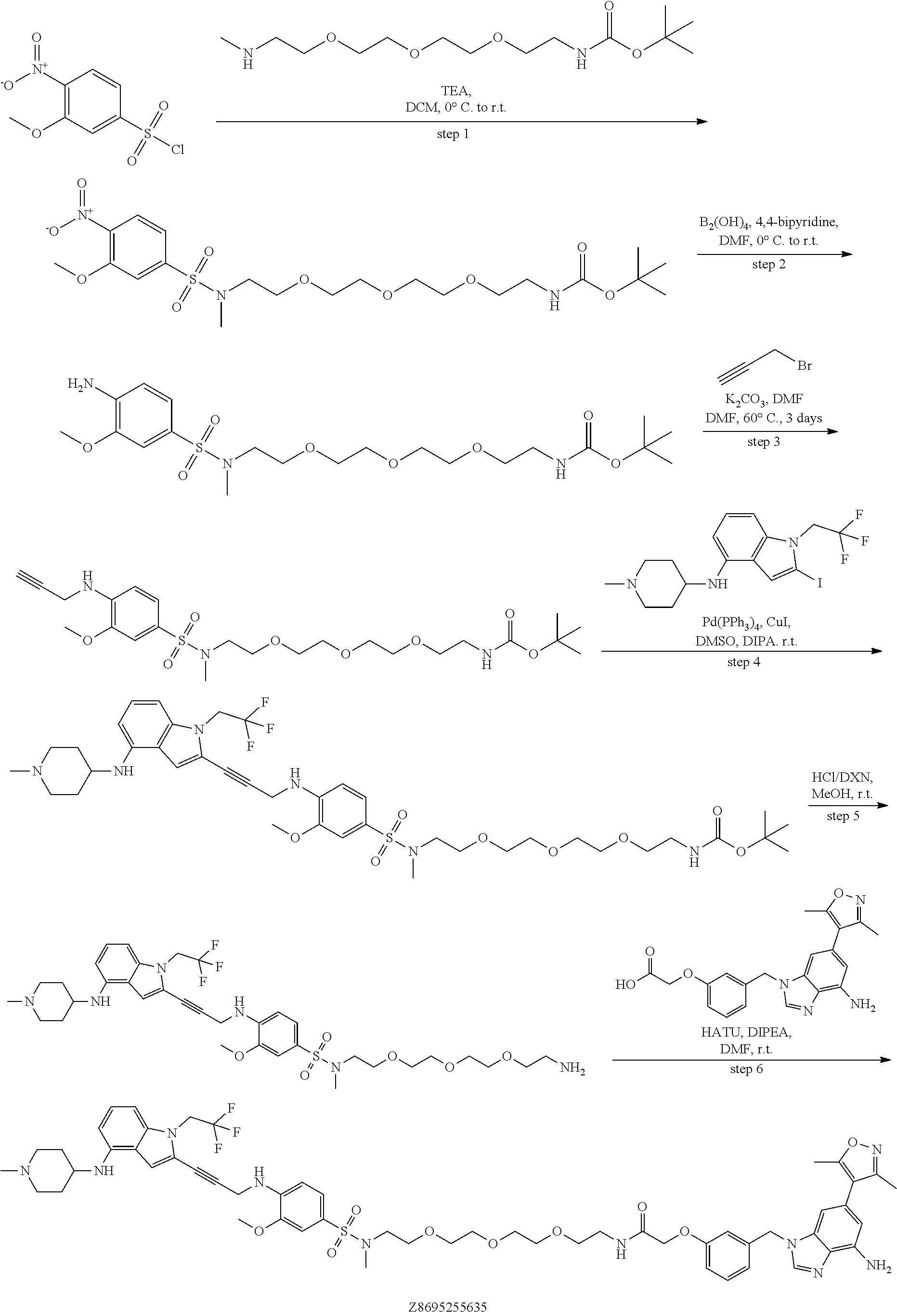

Step 1: The synthesis of tert-butyl N-[2-(2-{2-[2-(N-methyl-3-methoxy-4-nitrobenzenesulfonamido)ethoxy]ethoxy}ethoxy)ethyl]carbamate. A solution of 3-methoxy-4-nitrobenzene-1-sulfonyl chloride (3.0 g, 11.95 mmol) in THF (10 ml) was added to a solution of tert-butyl N-(5,8,11-trioxa-2-azatridecan-13-yl)carbamate (3.66 g, 11.95 mmol) and triethylamine (1.81 g, 17.93 mmol, 2.5 ml, 1.5 equiv) in DCM (30 ml) at 0° C. under nitrogen atmosphere. The mixture was warmed to RT and stirred for 12 h. Then the reaction mixture concentrated, diluted with EtOAc (60 ml) and washed with 1 M aqueour solution of NaHSO4 (10 ml), saturated solution of NaHCO3 (10 ml), brine, dried over anhydrous Na2SO4 and concentrated under reduced pressure to afford tert-butyl N-[2-(2-2-[2-(N-methyl-3-methoxy-4-nitrobenzenesulfonamido)ethoxy]ethoxyethoxy)ethyl]carbamate (5.0 g, 76.2% yield). The product obtained was of sufficient purity, so it was used in further experiments without any additional purification.

Step 2: The synthesis of tert-butyl N-[2-(2-{2-[2-(N-methyl-4-amino-3-methoxybenzenesulfonamido)ethoxy]ethoxy}ethoxy)ethyl]carbamate. (Dihydroxyboranyl)boronic acid (2.54 g, 28.18 mmol) was added portion wise to a solution of tert-butyl N-[2-(2-2-[2-(N-methyl-3-methoxy-4-nitrobenzenesulfonamido)ethoxy]ethoxyethoxy)ethyl]carbamate (4.9 g, 9.39 mmol) and 4,4-bipyridine in anhydrous DMF (60 mL), under inert atmosphere at 5° C. The resulting solution was stirred at room temperature for 1 hour. The reaction mixture was quenched by 10% solution of K2CO3 (240 mL), stirred up for 1 hour, extracted by EtOAc (3×100 ml), washed by water (3×50 ml), brine, dried over anhydrous Na2SO4 and concentrated under reduced pressure to afford tert-butyl N-[2-(2-2-[2-(N-methyl-4-amino-3-methoxybenzenesulfonamido)ethoxy]ethoxyethoxy)ethyl]carbamate (4.0 g, 74% yield) which was used in next step without purification.

Step 3: The synthesis of tert-butyl N-(2-{2-[2-(2-{N-methyl-3-methoxy-4-[(prop-2-yn-1-yl)amino]benzenesulfonamido}ethoxy)ethoxy]ethoxy}ethyl)carbamate. 3-bromoprop-1-yne (1.1 g, 9.36 mmol) and dipotassium carbonate (3.87 g, 28.07 mmol) were added to a solution of tert-butyl N-[2-(2-2-[2-(N-methyl-4-amino-3-methoxybenzenesulfonamido)ethoxy]ethoxyethoxy)ethyl]carbamate (4.6 g, 9.36 mmol) in DMF (75 ml), at room temperature under nitrogen atmosphere. The mixture was stirred at 60° C. for 12 h, then 3-bromoprop-1-yne (1.1 g, 9.36 mmol) was added and reaction was stirred at 60° C. for 8 h. The addition was continued until 80% conversion by LCMS was achieved (3 days total). Then the mixture diluted with EtOAc (150 ml) and washed with water (3×50 ml), brine, dried over anhydrous Na2SO4 and concentrated under reduced pressure to afford crude compound that was sent to column chromatography (ISCO®® Interchim; 120 g SiO2, petroleum ether/THF with acetonitrile from 10˜60% flow rate=46 mL/min) to afford tert-butyl N-(2-2-[2-(2-N-methyl-3-methoxy-4-[(prop-2-yn-1-yl)amino]benzenesulfonamidoethoxy)ethoxy]ethoxyethyl)carbamate (2.5 g, 45% yield).

Step 4: The synthesis of tert-butyl N-(2-{2-[2-(2-{N-methyl-3-methoxy-4-[(3-{4-[(1-methylpiperidin-4-yl)amino]-1-(2,2,2-trifluoroethyl)-1H-indol-2-yl}prop-2-yn-1-yl)amino]benzenesulfonamido}ethoxy)ethoxy]ethoxy}ethyl)carbamate. A solution of 2-iodo-N-(1-methylpiperidin-4-yl)-1-(2,2,2-trifluoroethyl)-1H-indol-4-amine (800.0 mg, 1.83 mmol), tert-butyl N-(2-2-[2-(2-N-methyl-3-methoxy-4-[(prop-2-yn-1-yl)amino]benzenesulfonamidoethoxy) ethoxy]ethoxyethyl)carbamate (1.82 g, 3.43 mmol), DiPA (5 mL), and dry DMSO (15 ml) was degassed by bubbling argon for 2 min. Tetrakis(triphenylphosphine)palladium (636.19 mg, 549.24 μmol) and CuI (34.75 mg, 183.08 μmol) were added and the mixture was again degassed by bubbling argon and was stirred under argon at RT for 12 h. The reaction mixture was diluted by saturated solution of EDTA in water (60 ml), extracted by EtOAc (3×50 ml). The combined organic layers was washed with water, brine, dried over anhydrous Na2SO4 and concentrated under reduced pressure then purified by column chromatography (ISCO®® Interchim; 80 g SiO2, Acetonitrile/Methanol with Methanol from 0˜95%, flow rate=60 mL/min, Rf=10-15CV) to afford tert-butyl N-(2-2-[2-(2-N-methyl-3-methoxy-4-[(3-4-[(1-methylpiperidin-4-yl)amino]-1-(2,2,2-trifluoroethyl)-1H-indol-2-ylprop-2-yn-1-yl)amino]benzenesulfonamidoethoxy)ethoxy]ethoxyethyl)carbamate (800 mg, 52% yield).

Step 5: The synthesis of N-(2-{2-[2-(2-aminoethoxy)ethoxy]ethoxy}ethyl)-3-methoxy-N-methyl-4-[(3-{4-[(1-methylpiperidin-4-yl)amino]-1-(2,2,2-trifluoroethyl)-1H-indol-2-yl}prop-2-yn-1-yl)amino]benzene-1-sulfonamide trihydrochloride. To a solution of tert-butyl N-(2-2-[2-(2-N-methyl-3-methoxy-4-[(3-4-[(1-methylpiperidin-4-yl)amino]-1-(2,2,2-trifluoroethyl)-1H-indol-2-ylprop-2-yn-1-yl)amino]benzenesulfonamidoethoxy)ethoxy]ethoxyethyl)carbamate (32.0 mg, 38.14 μmol) in MeOH (1 ml) was added HCl in Dioxane (2.2 M, 1 ml) at room temperature under nitrogen atmosphere. The mixture was stirred at room temperature for 1 hour. Then the reaction mixture concentrated under reduced pressure to afford crude N-(2-2-[2-(2-aminoethoxy)ethoxy]ethoxyethyl)-3-methoxy-N-methyl-4-[(3-4-[(1-methylpiperidin-4-yl)amino]-1-(2,2,2-trifluoroethyl)-1H-indol-2-ylprop-2-yn-1-yl)amino]benzene-1-sulfonamide trihydrochloride (28.0 mg, 64% yield) that was used in next step without purification.

Step 6: The synthesis of 2-(3-{[4-amino-6-(3,5-dimethyl-1,2-oxazol-4-yl)-1H-1,3-benzodiazol-1-yl]methyl}phenoxy)-N-(2-{2-[2-(2-{N-methyl-3-methoxy-4-[(3-{4-[(1-methylpiperidin-4-yl)amino]-1-(2,2,2-trifluoroethyl)-1H-indol-2-yl}prop-2-yn-1-yl)amino]benzenesulfonamido}ethoxy)ethoxy]ethoxy}ethyl)acetamide. A solution of N-(2-2-[2-(2-aminoethoxy)ethoxy]ethoxyethyl)-3-methoxy-N-methyl-4-[(3-4-[(1-methylpiperidin-4-yl)amino]-1-(2,2,2-trifluoroethyl)-1H-indol-2-ylprop-2-yn-1-yl)amino]benzene-1-sulfonamide (28.0 mg, 37.9 μmol), the 2-(3-[4-amino-6-(3,5-dimethyl-1,2-oxazol-4-yl)-1H-1,3-benzodiazol-1-yl]methylphenoxy)acetic acid (14.88 mg, 37.91 μmol), [(dimethylamino)(3H-[1,2,3]triazolo[4,5-b]pyridin-3-yloxy)methylidene]dimethylazanium; hexafluoro-lambda5-phosphanuide (18.73 mg, 49.28 μmol) and ethylbis(propan-2-yl)amine (48.96 mg, 379.07 μmol) in dry DMF (1 ml) was stirred under argon at RT for 12 h. The mixture was purified by HPLC (40-40-90% 0-1-6 min H2O/ACN/NH4OH flow: 60 ml/min; column:XBridge C18 OBD 30×100 mm 5 um) to afford 2-(3-[4-amino-6-(3,5-dimethyl-1,2-oxazol-4-yl)-1H-1,3-benzodiazol-1-yl]methylphenoxy)-N-(2-2-[2-(2-N-methyl-3-methoxy-4-[(3-4-[(1-methylpiperidin-4-yl)amino]-1-(2,2,2-trifluoroethyl)-1H-indol-2-ylprop-2-yn-1-yl)amino]benzenesulfonamidoethoxy)ethoxy]ethoxyethyl)acetamide (14.7 mg, 98.0% purity, 12.94 μmol, 34.1% yield) with the following spectra data: LCMS (6 min, 99% by UV 215 nm, RT 2.891, M+1=114.2).

### Example 34: Synthesis of 2-(3-{[5-(3,5-dimethyl-1,2-oxazol-4-yl)-2-oxo-1,2-dihydropyridin-1-yl]methyl}phenoxy)-N-{2-[2-(2-{4-[(3-{4-[(1-methylpiperidin-4-yl)amino]-1-(2,2,2-trifluoroethyl)-1H-indol-2-yl}prop-2-yn-1-yl)amino]phenyl}ethoxy)ethoxy]ethyl}acetamide (Z8736893433)

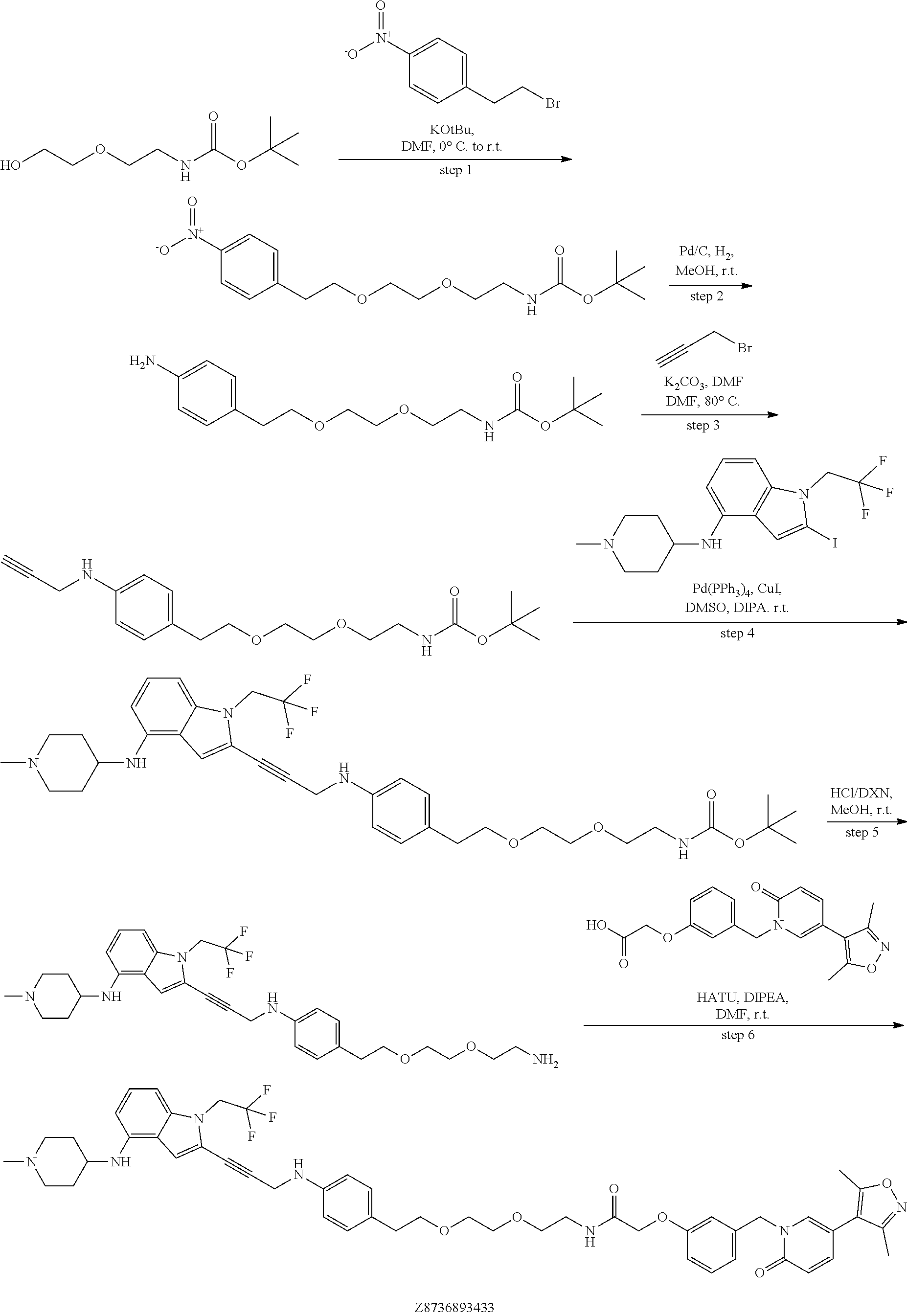

Step 1: The synthesis of tert-butyl N-(2-{2-[2-(4-nitrophenyl)ethoxy]ethoxy}ethyl)carbamate. To a solution of tert-butyl N-[2-(2-hydroxyethoxy)ethyl]carbamate (4.45 g, 21.69 mmol) in anhydrous THF (200 ml) was added potassium 2-methylpropan-2-olate (4.86 g, 43.39 mmol) portion wise at 0° C. The reaction mixture was stirred for 2 hours under inert atmosphere, then a solution of 1-(2-bromoethyl)-4-nitrobenzene (4.97 g, 21.69 mmol) in THF (20 ml) was added dropwise at 0° C. The resulting solution was then allowed to warm up to room temperature and left while stirring for 12 hours. The reaction mixture was poured onto water and extracted with ethyl acetate (3×150 ml). The organic layers were combined, washed with water (3×100 ml), brine (100 ml), dried over anhydrous Na2SO4 and filtered. The filtrate collected was concentrated under reduced pressure and the crude obtained was subjected for flash chromatography (ISCO®: Interchim; 220 g SiO2, CHCl3/CH3CN with CH3CN from 0˜95%, flow rate=100 mL/min) to afford the desired tert-butyl N-(2-2-[2-(4-nitrophenyl)ethoxy]ethoxyethyl)carbamate (350 mg, 4.1% yield) as yellow solid.

Step 2: The synthesis of tert-butyl N-(2-{2-[2-(4-aminophenyl)ethoxy]ethoxy}ethyl)carbamate. 5% Pd/C (0.2 g) was added to a solution of tert-butyl N-(2-2-[2-(4-nitrophenyl)ethoxy]ethoxyethyl)carbamate (170.0 mg, 479.69 μmol) in MeOH (20 ml). The resulting mixture was hydrogenated at 1 atm and ambient temperature overnight. The reaction mixture was filtered and the filtrate was concentrated under reduced pressure to afford the desired tert-butyl N-(2-2-[2-(4-aminophenyl)ethoxy]ethoxyethyl)carbamate (140.0 mg, 76.3% yield) as yellow solid. The crude product was used in further experiments without any additional purification.

Step 3: The synthesis of tert-butyl N-{2-[2-(2-{4-[(prop-2-yn-1-yl)amino]phenyl}ethoxy) ethoxy]ethyl}carbamate. To a solution of tert-butyl N-(2-2-[2-(4-aminophenyl)ethoxy]ethoxyethyl)carbamate (140 mg, 432 μmol) in anhydrous DMF (5 ml) was added dipotassium carbonate (119 mg, 860 μmol) and then under inert atmosphere 3-bromoprop-1-yne (50.7 mg, 430 μmol) dropwise at 0° C. The resulting solution was left to warm up to room temperature while stirring for 18 hours. The reaction mixture was diluted with saturated solution of NH4Cl (50 ml) and extracted with ethyl acetate (3×50 ml). The organic layers were combined, washed with water (3×50 ml), brine (50 ml), dried over anhydrous sodium sulfate and filtered. The filtrate was concentrated under reduced pressure and the crude obtained was subjected for flash chromatography (ISCO® Companion combiflash; 40 g SiO2, Chloroform/Acetonitrile with Acetonitrile from 0˜95%, flow rate=40 mL/min) to afford the desired tert-butyl N-2-[2-(2-4-[(prop-2-yn-1-yl)amino]phenylethoxy)ethoxy]ethylcarbamate (37.0 mg, 22% yield) as yellow oil.

Step 4: The synthesis of tert-butyl N-{2-[2-(2-{4-[(3-{4-[(1-methylpiperidin-4-yl)amino]-1-(2,2,2-trifluoroethyl)-1H-indol-2-yl}prop-2-yn-1-yl)amino]phenyl}ethoxy)ethoxy]ethyl}carbamate. Tetrakis(triphenylphosphine)palladium (38.28 mg, 33.05 μmol) was added to a solution of 2-iodo-N-(1-methylpiperidin-4-yl)-1-(2,2,2-trifluoroethyl)-1H-indol-4-amine (79.44 mg, 181.75 μmol), tert-butyl N-2-[2-(2-4-[(prop-2-yn-1-yl)amino]phenylethoxy)ethoxy]ethylcarbamate (59.89 mg, 165.23 μmol), CuI (3.14 mg, 16.52 μmol) in DMSO/i-Pr2NH (5/1, 4 ml) under inert atmosphere. The resulting solution was stirred at RT for 18 hours. The reaction mixture was then poured onto water (50 ml) and extracted with ethyl acetate (3×50 ml). The organic layers were combined, washed with water (3×50 ml), brine (50 ml), dried over anhydrous Na2SO4 and filtered. The filtrate collected was concentrated under reduced pressure and the crude obtained was subjected for flash chromatography (ISCO®: Interchim; 40 g SiO2, CH3CN/methanol with methanol from 0˜100%, flow rate=40 mL/min, Rf=10-15 CV.) to afford the desired tert-butyl N-2-[2-(2-4-[(3-4-[(1-methylpiperidin-4-yl)amino]-1-(2,2,2-trifluoroethyl)-1H-indol-2-ylprop-2-yn-1-yl)amino]phenylethoxy)ethoxy]ethylcarbamate (40.0 mg, 18% yield) as yellow oil.

Step 5: The synthesis of 2-{3-[(4-{2-[2-(2-aminoethoxy)ethoxy]ethyl}phenyl)amino]prop-1-yn-1-yl}-N-(1-methylpiperidin-4-yl)-1-(2,2,2-trifluoroethyl)-1H-indol-4-amine hydrochloride. HCl (10% in dioxane, 0.18 ml) was added to solution of tert-butyl N-2-[2-(2-4-[(3-4-[(1-methylpiperidin-4-yl)amino]-1-(2,2,2-trifluoroethyl)-1H-indol-2-ylprop-2-yn-1-yl)amino]phenylethoxy) ethoxy]ethylcarbamate (40 mg, 58.54 μmol) in MeOH (4 ml). The reaction mixture was stirred at RT for 18 hours. After that period the mixture was concentrated under reduced pressure to afford the desired 2-3-[(4-2-[2-(2-aminoethoxy)ethoxy]ethylphenyl)amino]prop-1-yn-1-yl-N-(1-methylpiperidin-4-yl)-1-(2,2,2-trifluoroethyl)-1H-indol-4-amine hydrochloride (40.0 mg, 43.0% purity, 28.28 μmol, 48.3% yield) as yellow solid. The product was used in further experiments without any additional purification.

Step 6: The synthesis of 2-(3-{[5-(3,5-dimethyl-1,2-oxazol-4-yl)-2-oxo-1,2-dihydropyridin-1-yl]methyl}phenoxy)-N-{2-[2-(2-{4-[(3-{4-[(1-methylpiperidin-4-yl)amino]-1-(2,2,2-trifluoroethyl)-1H-indol-2-yl}prop-2-yn-1-yl)amino]phenyl}ethoxy)ethoxy]ethyl}acetamide. (1H-1,2,3-benzotriazol-1-yloxy)tris(dimethylamino)phosphanium; hexafluoro-lambda5-phosphanuide (43.6 mg, 98.6 μmol), ethylbis(propan-2-yl)amine (84.9 mg, 658 μmol) were added to a solution of 2-(3-[5-(3,5-dimethyl-1,2-oxazol-4-yl)-2-oxo-1,2-dihydropyridin-1-yl]methylphenoxy)acetic acid (23.28 mg, 65.75 μmol) in DMF (4 ml) at r.t. Then after 5 min 2-3-[(4-2-[2-(2-aminoethoxy)ethoxy]ethylphenyl)amino]prop-1-yn-1-yl-N-(1-methylpiperidin-4-yl)-1-(2,2,2-trifluoroethyl)-1H-indol-4-amine hydrochloride (40.0 mg, 65.77 μmol) was added. The resulting mixture was stirred at room temperature overnight. The reaction mixture solution was subjected for prep HPLC purification without any further work-up (33-40-65-100% 0-2-7-7.1 30 ml/min H2O-ACN+NH3; column Xbridge C18 5 uM 19*100 mm) to result in the desired 2-(3-[5-(3,5-dimethyl-1,2-oxazol-4-yl)-2-oxo-1,2-dihydropyridin-1-yl]methylphenoxy)-N-2-[2-(2-4-[(3-4-[(1-methylpiperidin-4-yl)amino]-1-(2,2,2-trifluoroethyl)-1H-indol-2-ylprop-2-yn-1-yl)amino]phenylethoxy)ethoxy]ethylacetamide (5.5 mg, 9.1% yield) as yellow gum. with the following spectra data: LCMS (6 min, 98.7% by UV 215 nm, RT 3.047, M+1=909.4), HNMR, DMSO-d6 (H4891521—purity 95+%).

### Example 35: Synthesis of 2-(3-{[5-(3,5-dimethyl-1,2-oxazol-4-yl)-2-oxo-1,2-dihydropyridin-1-yl]methyl}phenoxy)-N-(2-{2-[2-(2-{4-[(3-{4-[(1-methylpiperidin-4-yl)amino]-1-(2,2,2-trifluoroalkyl)-1H-indol-2-yl}prop-2-yn-1-yl)amino]benzenesulfonyl}ethoxy)ethoxy]ethoxy}ethyl)acetamide (Z8604784280)

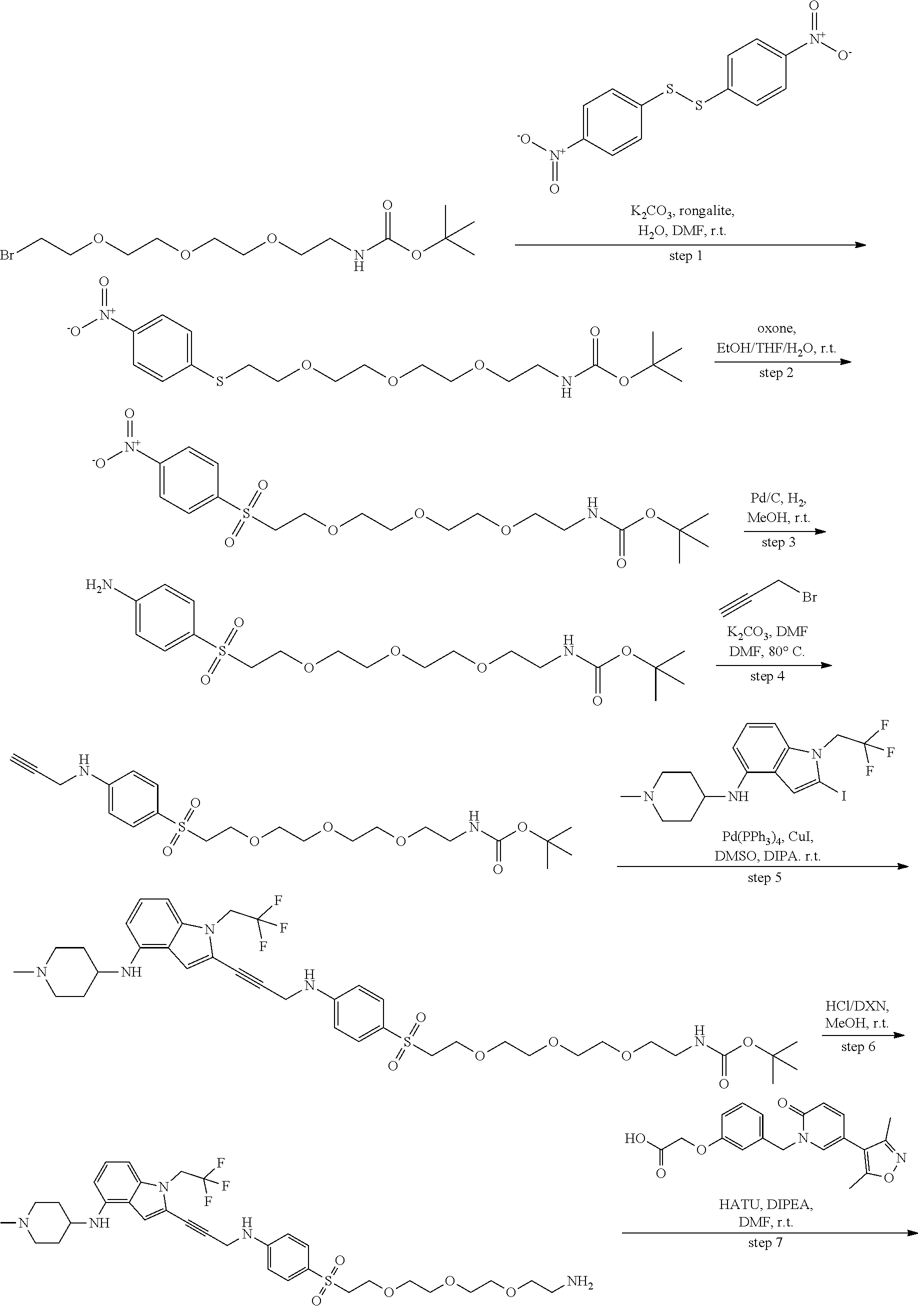

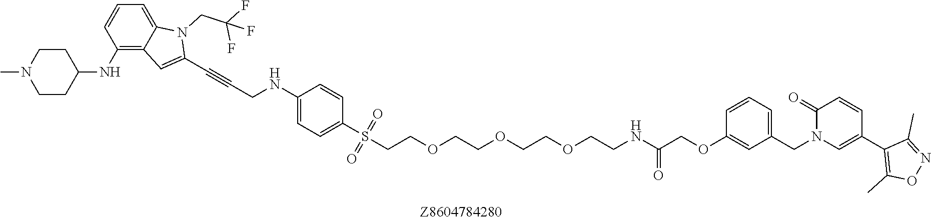 [SMILES: Cc1noc(C)c1-c1ccc(=O)n(Cc2cccc(OCC(=O)NCCOCCOCCOCCS(=O)(=O)c3ccc(NCC#Cc4cc5c(NC6CCN(C)CC6)cccc5n4CC(F)(F)F)cc3)c2)c1]

Step 1: The synthesis of tert-butyl N-{2-[2-(2-{2-[(4-nitrophenyl)sulfanyl]ethoxy}ethoxy) ethoxy]ethyl}carbamate. To a solution of 1-nitro-4-[(4-nitrophenyl)disulfanyl]benzene (1.0 g, 3.25 mmol) in DMF (30 ml), was added tert-butyl N-(2-2-[2-(2-bromoethoxy)ethoxy]ethoxyethyl)carbamate (2.1 g, 5.9 mmol), sodium hydroxymethanesulfinate (1.04 g, 8.86 mmol), dipotassium carbonate (814.31 mg, 5.9 mmol) at room temperature under nitrogen atmosphere. The mixture was stirred at RT for 12 h. LCMS analysis of the reaction mixture showed full conversion to the desired product. Then the mixture diluted with EtOAc (150 ml) and washed with water (3×50 ml), brine, dried over anhydrous Na2SO4 and concentrated under reduced pressure to afford tert-butyl N-2-[2-(2-2-[(4-nitrophenyl)sulfanyl]ethoxyethoxy)ethoxy]ethylcarbamate (2.5 g, 77% yield) that was used in next step without purification.

Step 2: The synthesis of tert-butyl N-[2-(2-{2-[2-(4-nitrobenzenesulfonyl)ethoxy]ethoxy}ethoxy)ethyl]carbamate. To a solution of tert-butyl N-2-[2-(2-2-[(4-nitrophenyl)sulfanyl]ethoxyethoxy)ethoxy]ethylcarbamate (2.5 g, 5.81 mmol) in EtOH/THF/H2O (1:1:1, 60 ml), was added oxone (7.13 g, 11.61 mmol) at room temperature under nitrogen atmosphere. The mixture was stirred at room temperature for 16 h. Then the reaction mixture was filtered, diluted with water and extracted with ethyl acetate. The organic layer was washed with brine, dried over anhydrous Na2SO4 and concentrated under reduced pressure to afford crude compound that was purified by column chromatography (ISCO®® Interchim; 40 g SiO2, chloroform/acetonitrile with acetonitrile from 0˜100%, flow rate=40 mL/min) to afford tert-butyl N-[2-(2-2-[2-(4-nitrobenzenesulfonyl)ethoxy]ethoxyethoxy)ethyl]carbamate (700 mg, 23.5% yield)

Step 3: The synthesis of tert-butyl N-[2-(2-{2-[2-(4-aminobenzenesulfonyl)ethoxy]ethoxy}ethoxy)ethyl]carbamate. To solution of the tert-butyl N-[2-(2-2-[2-(4-nitrobenzenesulfonyl)ethoxy]ethoxyethoxy)ethyl]carbamate (2.0 g, 4.32 mmol, 1, 1.0 equiv) in 40 ml of MeOH was added palladium (357.23 mg, 3.37 mmol) and mixture was degassed by vacuum pump for 2 min. Then atmosphere was changed to H2 by alternating between vacuum and H2 twice. The reaction was stirred at RT under a hydrogen atmosphere, after which time the mixture was filtered through celite, concentrated under reduced pressure to afford tert-butyl N-[2-(2-2-[2-(4-aminobenzenesulfonyl)ethoxy]ethoxyethoxy)ethyl]carbamate (1.1 g, 64.1% yield). The crude product was used in the next step without purification.

Step 4: The synthesis of tert-butyl N-(2-{2-[2-(2-{4-[(prop-2-yn-1-yl)amino]benzenesulfonyl}ethoxy)ethoxy]ethoxy}ethyl)carbamate. To a solution of tert-butyl N-[2-(2-2-[2-(4-aminobenzenesulfonyl)ethoxy]ethoxyethoxy)ethyl]carbamate (1.0 g, 2.31 mmol) in DMF (10 ml), was added 3-bromoprop-1-yne (272.69 mg, 2.31 mmol) and dipotassium carbonate (956.58 mg, 6.94 mmol) at room temperature under nitrogen atmosphere. The mixture was stirred at 80° C. for 12 h. After that another portion of 3-bromoprop-1-yne (272.69 mg, 2.31 mmol) was added and reaction was stirred at 80° C. for 8 h. The addition was continued until 80% conversion by LCMS was achieved. Then the mixture diluted with EtOAc (30 ml) and washed with water (3×10 ml), brine, dried over anhydrous Na2SO4 and concentrated under reduced pressure to afford crude compound that was purified (ISCO®® Interchim; 40 g SiO2, chloroform/acetonitrile with acetonitrile from 0˜100%, flow rate=40 mL/min) to afford tert-butyl N-(2-2-[2-(2-4-[(prop-2-yn-1-yl)amino]benzenesulfonylethoxy)ethoxy]ethoxyethyl)carbamate (90.0 mg, 7% yield)

Step 5: The synthesis of tert-butyl N-(2-{2-[2-(2-{4-[(3-{4-[(1-methylpiperidin-4-yl)amino]-1-(2,2,2-trifluoroethyl)-1H-indol-2-yl}prop-2-yn-1-yl)amino]benzenesulfonyl}ethoxy)ethoxy]ethoxy}ethyl) carbamate. A solution of 2-iodo-N-(1-methylpiperidin-4-yl)-1-(2,2,2-trifluoroethyl)-1H-indol-4-amine (62.76 mg, 143.61 μmol), tert-butyl N-(2-2-[2-(2-4-[(prop-2-yn-1-yl)amino]benzenesulfonylethoxy)ethoxy]ethoxyethyl)carbamate (90.0 mg, 191.25 μmol), DiPA (1 mL), and dry DMSO (3 ml) was degassed by bubbling argon for 2 min. Tetrakis(triphenylphosphine)palladium (33.27 mg, 28.72 μmol) and CuI (2.73 mg, 14.36 μmol) were added and the mixture was again degassed by bubbling argon and was stirred under argon at RT for 12 h. The reaction mixture was diluted by saturated solution of EDTA in water (12 ml), extracted by EtOAc (3×10 ml). The combined organic layers were washed with water, brine, dried over anhydrous Na2SO4 and concentrated under reduced pressure. The crude compound purified by HPLC (40-40-90% 0-1-5 min H2O/ACN/NH4OH flow: 60 ml/min; column: XBridge C18 OBD 30×100 mm 5 um) to afford tert-butyl N-(2-2-[2-(2-4-[(3-4-[(1-methylpiperidin-4-yl)amino]-1-(2,2,2-trifluoroethyl)-1H-indol-2-ylprop-2-yn-1-yl)amino]benzenesulfonylethoxy)ethoxy]ethoxyethyl)carbamate (45.0 mg, 40% yield)

Step 6: The synthesis of 2-(3-{[4-(2-{2-[2-(2-aminoethoxy)ethoxy]ethoxy}ethanesulfonyl) phenyl]amino}prop-1-yn-1-yl)-N-(1-methylpiperidin-4-yl)-1-(2,2,2-trifluoroethyl)-1H-indol-4-amine trihydrochloride. HCl in Dioxane (2.2 M, 1 ml) was added to a solution of tert-butyl N-(2-2-[2-(2-4-[(3-4-[(1-methylpiperidin-4-yl)amino]-1-(2,2,2-trifluoroethyl)-1H-indol-2-ylprop-2-yn-1-yl)amino]benzenesulfonylethoxy)ethoxy]ethoxyethyl)carbamate (45.0 mg, 57.7 μmol) in MeOH (1 ml), at room temperature under nitrogen atmosphere. The reaction mixture was stirred at room temperature for 1 hour, then was concentrated under reduced pressure to afford crude 2-(3-[4-(2-2-[2-(2-aminoethoxy)ethoxy]ethoxyethanesulfonyl)phenyl]aminoprop-1-yn-1-yl)-N-(1-methylpiperidin-4-yl)-1-(2,2,2-trifluoroethyl)-1H-indol-4-amine trihydrochloride (450.0 mg) that was used right away in next step without purification.

Step 7: The synthesis of 2-(3-{[5-(3,5-dimethyl-1,2-oxazol-4-yl)-2-oxo-1,2-dihydropyridin-1-yl]methyl}phenoxy)-N-(2-{2-[2-(2-{4-[(3-{4-[(1-methylpiperidin-4-yl)amino]-1-(2,2,2-trifluoroethyl)-1H-indol-2-yl}prop-2-yn-1-yl)amino]benzenesulfonyl}ethoxy)ethoxy]ethoxy}ethyl)acetamide. A solution of the 2-(3-[4-(2-2-[2-(2-aminoethoxy)ethoxy]ethoxyethanesulfonyl)phenyl]aminoprop-1-yn-1-yl)-N-(1-methylpiperidin-4-yl)-1-(2,2,2-trifluoroethyl)-1H-indol-4-amine trihydrochloride (40.0 mg, 50.69 μmol), the 2-(3-[5-(3,5-dimethyl-1,2-oxazol-4-yl)-2-oxo-1,2-dihydropyridin-1-yl]methylphenoxy)acetic acid (17.95 mg, 50.68 μmol), the [(dimethylamino)(3H-[1,2,3]triazolo[4,5-b]pyridin-3-yloxy)methylidene]dimethylazanium; hexafluoro-lambda5-phosphanuide (28.9 mg, 76.02 μmol) and the ethylbis(propan-2-yl)amine (65.46 mg, 506.82 μmol) in dry DMF (1 ml) was stirred under argon at RT for 12 hours. The mixture was purified by HPLC (40-40-70% 0-1-8 min H2O/ACN/NH4OH flow: 60 ml/min; column: XBridge C18 OBD 100×30 mm 5 um) to afford 2-(3-[5-(3,5-dimethyl-1,2-oxazol-4-yl)-2-oxo-1,2-dihydropyridin-1-yl]methylphenoxy)-N-(2-2-[2-(2-4-[(3-4-[(1-methylpiperidin-4-yl)amino]-1-(2,2,2-trifluoroethyl)-1H-indol-2-ylprop-2-yn-1-yl)amino]benzenesulfonylethoxy)ethoxy]ethoxyethyl)acetamide (24.8 mg, 48.2% yield) with the following spectra data: LCMS (6 min, >99% by UV 215 nm, RT 2.942, M+1=1017.4).

### Example 36: p53 Phosphorylation

OmniTAC compounds are created with targeting ligands for p53 and recruiting ligands for proteins capable of modulating p53 activity. Linkers connect the targeting and recruiting ligands. A screening of the compounds determines the best binders to certain proteins and p53. The compounds are then tested in vitro. Cells are cultured at 37° C. at proper humidity levels. A control group is created that only contains cells and vehicle treatment. A treatment group with OmniTAC is tested, and compared to the control group.

Selected OmniTAC compounds are added to the treated cells at various concentrations, for example, 250 nM, 500 nM, and 1,000 nM. The compounds are added incubated with the cells for various time intervals such as 6, 12, and 24 hours. Following incubation, cells are harvested, and proteins will be isolated from the cells. Proteins are stored with phosphatase inhibitors to maintain the phosphorylation status of the proteins.

Western blots are performed with p53 and phospho-p53 antibodies to measure levels of p53 relative to phosphorylated p53. Antibodies specific to phosphorylation at certain residues will also be used. Data are expected to show outputs of phosphorylated p53 at various time intervals and concentrations. Effective OmniTAC compounds increase p53 phosphorylation relative to the control group. The most effective OmniTAC compounds will be tested further in animal disease models which include p53 dephosphorylation such as certain cancer models

### Example 37: Western Blot of Protein Induction of p21 and MDM2 Through p53 Mutant Activation in H1299 Cells

OmniTAC compounds are created that include targeting ligands for p53 and recruiting ligands for proteins capable of modulating p53 activity. Linkers connect the targeting and recruiting ligands. A screening of the compounds determine the best binders to certain various proteins and p53. The compounds will then be tested in vitro. Cells are cultured at 37° C. at proper humidity levels. A control group is created that only contains cells and vehicle treatment. A treatment group with OmniTAC is tested, and compared to the control group.

Selected OmniTAC compounds are to the treated cells at various concentrations, for example, 250 nM, 500 nM, and 1,000 nM. The compounds are incubated with the cells for various time intervals such as 6, 12, and 24 hours. Following incubation, cells are harvested, and proteins are isolated from the cells.

Western blots are performed with p53 and p53 downstream target antibodies to measure levels of p53 relative to p53-dependent pro-apoptotic, cell-cycle arrest or negative regulator proteins. In some instances, antibodies specific to p21, PUMA, Noxa, Bax, and/or MDM2 are used. Data are expected to show outputs of upregulated p53-dependent protein levels for pro-apoptotic, cell-cycle arrest or negative regulator proteins at various time intervals and concentrations. Effective OmniTAC compounds increase p53 downstream target proteins relative to the control group. The most effective OmniTAC compounds are tested further in animal disease models.

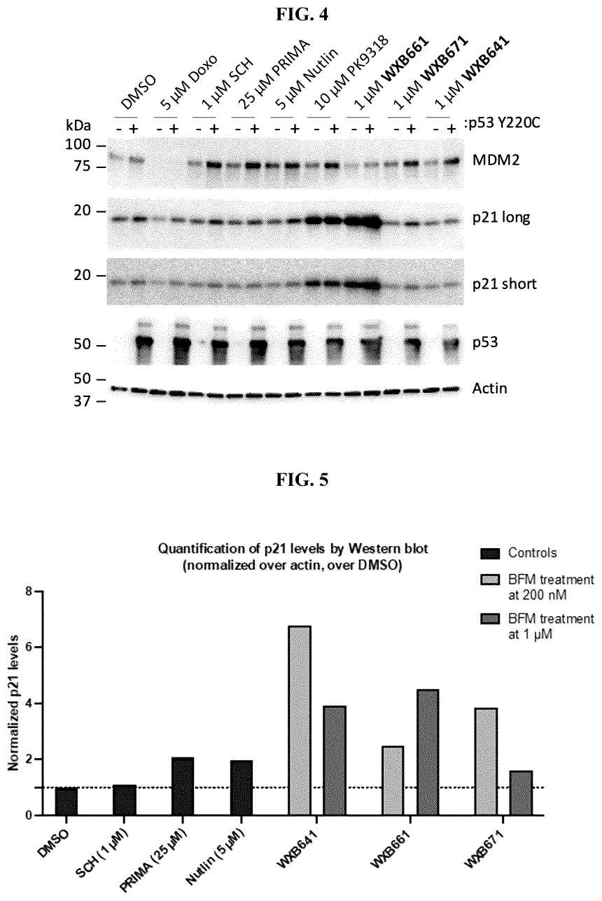

For instance, H1299 cells (p53 null or p53 Y220C) were treated with DMSO, control compounds or BFMs (1 μM) for 24 h. The cell lysates were analyzed by SDS-PAGE gel electrophoresis and Western blot. The samples were visualized using the following secondary antibodies (anti-p53 hrp, 1:1000; anti-actin hrp, 1:2000; anti-p21, 1:1000, anti-MDM2, 1:1000). Results of the Western blot are shown in FIG. 4.

### Example 38: Western Blot of Protein Induction of p21 Through p53 Mutant Activation in Huh7

OmniTAC compounds are created that include targeting ligands for p53 and recruiting ligands for proteins capable of modulating p53 activity. Linkers connect the targeting and recruiting ligands. A screening of the compounds determines the best binders to various modifier proteins and p53. The compounds will then be tested in vitro. Cells are cultured at 37° C. at proper humidity levels. A control group is created that only contains cells and vehicle treatment. A treatment group with OmniTAC is tested, and compared to the control group.

Selected OmniTAC compounds are added to the treated cells at various concentrations, for example, 250 nM, 500 nM, and 1,000 nM. The compounds are incubated with the cells for various time intervals such as 6, 12, and 24 hours. Following incubation, cells are harvested, and proteins are isolated from the cells.

Western blots are performed with p53 and p53 downstream target antibodies to measure levels of p53 relative to p53-dependent pro-apoptotic, cell-cycle arrest or negative regulator proteins. In particular, antibodies specific to p21, PUMA, Noxa, Bax, and MDM2 are used. Data are expected to show outputs of upregulated p53-dependent protein levels for pro-apoptotic, cell-cycle arrest or negative regulator proteins at various time intervals and concentrations. Effective OmniTAC compounds increase p53 downstream target proteins relative to the control group. The most effective OmniTAC compounds will be tested further in animal disease models.

For instance, Huh7 cells (endogenous mutant p53) were treated with DMSO, control compounds or BFMs (1 or 0.2 μM) for 24 h. The cell lysates were analyzed by SDS-PAGE gel electrophoresis and Western blot. The samples were visualized using the following secondary antibodies (anti-p53 hrp, 1:1000; anti-actin hrp, 1:2000; anti-p21, 1:1000, anti-MDM2, 1:1000). Results of the Western blot quantification are shown in FIG. 5.

### Example 39: Activation of p53 Mutants for Two Bifunctional Molecules WXB661 and WXB672

OmniTAC compounds are created that include targeting ligands for p53 and recruiting ligands for proteins capable of modulating p53 activity. Linkers connect the targeting and recruiting ligands. A screening of the compounds determines the best binders to certain modifier proteins and p53 using a stably expressed p53-luciferase reporter gene. A lentivirus vector containing a p53 recognition element, a minimal promoter and luciferase is used to create stable p53 luciferase cells. The compounds are then tested in vitro. Cells are cultured at 37° C. at proper humidity levels. A control group is created that only contains cells and vehicle treatment. A treatment group with OmniTAC is tested, and compared to the control group.

Selected OmniTAC compounds are added to the treated cells at various concentrations, for example, 250 nM, 500 nM, and 1,000 nM. The compounds are incubated with the cells for various time intervals such as 6, 12, and 24 hours. Following incubation, p53 activity is measured by monitoring luciferase activity.

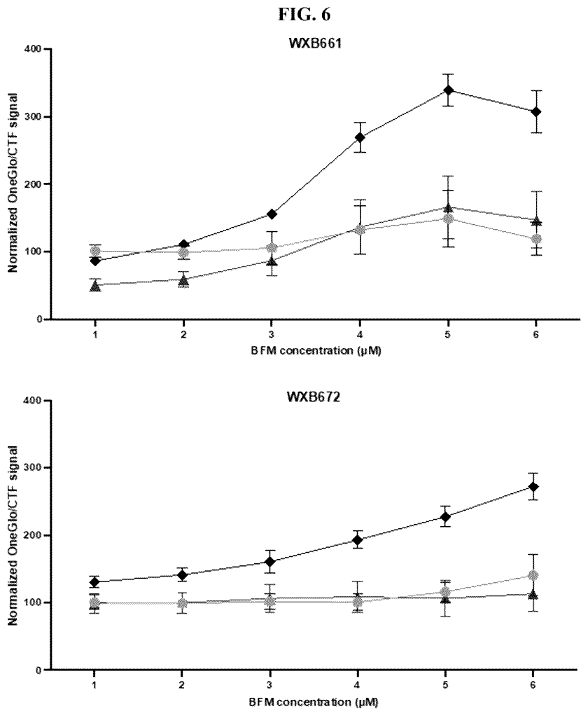

For instance, H1299 cells (4,000 cells per well) were treated with DMSO, control compounds or BFMs (0.12-12.5 μM) for 24 h. Each sample was run in triplicate. p53 activation was measured using p53-luciferase reporter assay utilizing Promega's ONE-Glo™ Luciferase protocol as the readout. Cell numbers were normalized using Promega's CellTiter Fluor™ protocol. Results are shown in FIG. 6.

### Example 40: Cell Viability Assays Showing Bifunctional Molecule Induced Cell Death

OmniTAC compounds are created that include targeting ligands for p53 and recruiting ligands for proteins capable of modulating p53 activity. Linkers connect the targeting and recruiting ligands. A screening of the compounds determines the best binders to certain modifier proteins and p53 using a cell viability assay. Cell viability in p53 WT, p53 mutant and/or p53 null cell lines is compared. The compounds are then tested in vitro. Cells are cultured at 37° C. at proper humidity levels. A control group is created that only contains cells and vehicle treatment. A treatment group with OmniTAC is tested, and compared to the control group.

Selected OmniTAC compounds are added to the treated cells at various concentrations, for example, 250 nM, 500 nM, and 1,000 nM. The compounds are incubated with the cells for various time intervals such as 24, 48, and 72 hours. Following incubation, cell viability is measured by monitoring fluorescence.

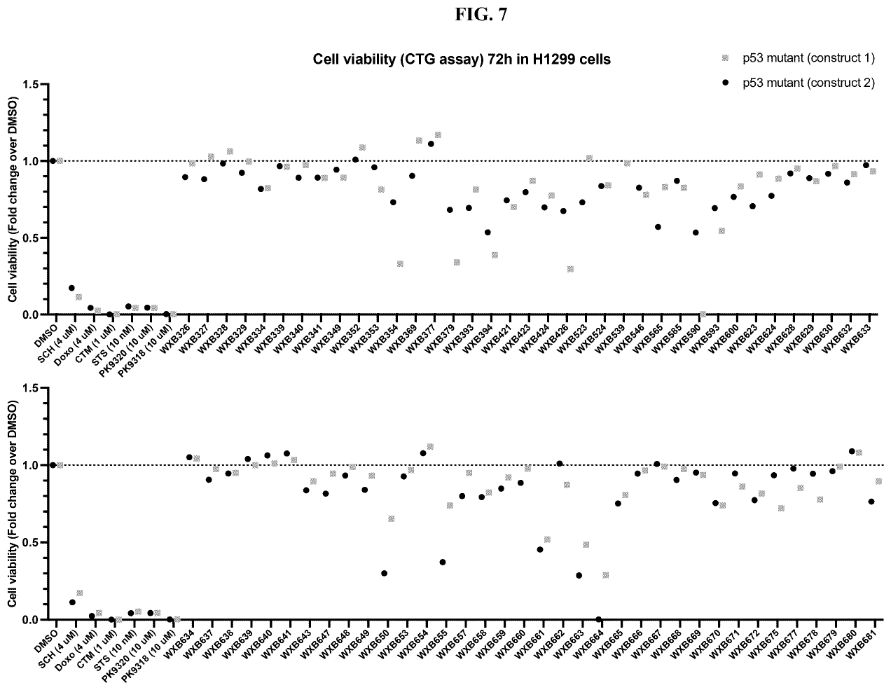

For instance, H1299 cells ((p53 null or p53 Y220C), 500 cells/well) were treated with DMSO, control compounds or BFMs (1 μM) for 72 h. Cell viability was measured through measuring the amount of available ATP via luciferase activity as detailed in the Promega's CellTiter Glo™ protocol: following addition of the CTG reagents the plate was incubated for 10 mins in the dark, prior to measuring the luminescence data for each well (0.3s integration time). Results are shown in FIG. 7.

### Example 41: Activation of Mutant GBA Using Bifunctional Molecules

OmniTAC compounds are created that include targeting ligands for p53 and recruiting ligands for proteins capable of modulating GBA activity. Linkers connect the targeting and recruiting ligands. A screening of the compounds determines the best binders to certain modifier proteins and GBA using an enzymatic assay. The compounds will then be tested in vitro. Cells are cultured at 37° C. at proper humidity levels. A control group is created that only contains cells and vehicle treatment. A treatment group with OmniTAC is tested, and compared to the control group.

Selected OmniTAC compounds are added to the treated cells at various concentrations, for example, 250 nM, 500 nM, and 1,000 nM. The compounds are incubated with the cells for various time intervals such as 24, 48 and 72 hours. Following incubation, GBA activity is measured in live cells by monitoring β-glucocerebrosidase activity through fluorescence.

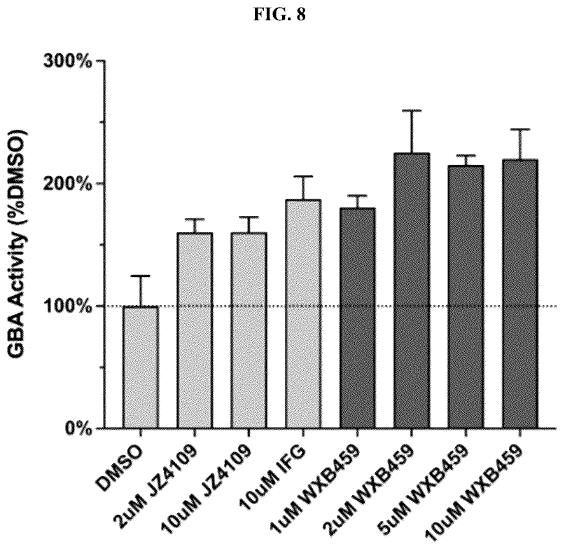

For instance, Gaucher's disease patient derived fibroblasts (GBA N370S/-) were treated with DMSO, control compounds or BFMs (1 μM) for 24 h. GBA activity was assessed in live cells using a fluorogenic β-glucocerebrosidase activity assay with 4-Methylumbelliferyl-β-D-glucopyranoside as the substrate. Results are shown in FIG. 8.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A modifier protein targeting chimeric (OmniTAC) compound comprising a targeting ligand, a recruiting ligand, and a linker; wherein the targeting ligand is attached to the recruiting ligand via the linker;
wherein the targeting ligand is configured to bind to a target protein, and the recruiting ligand is configured to bind to a modifier protein such that the modifier protein induces a change to the target protein, or wherein the recruiting ligand is configured to bind to a nucleic acid such that the target protein is targeted to the nucleic acid; and
wherein the modifier protein comprises a non-degradative protein that induces activation, stabilization, or corrects misfolding of the target protein.

2. The compound of claim 1, wherein the target protein comprises a tumor suppressor, metabolic enzyme, protein aggregate, or haploinsufficient protein.

3. The compound of claim 11, wherein the target protein comprises P53, VHL, or GBA.

4. (canceled)

5. The compound of claim 1, wherein the recruiting ligand is derived from any small molecule, or analogues of any small molecule in FIG. 9.

6. The compound of claim 1, wherein the modifier protein comprises an epigenetic modifier, epigenetic reader, chaperone, or nuclear targeting protein.

7. The compound of claim 1, wherein the modifier protein comprises a acylase, deacylase, organelle specific protein, kinase, phosphatase, palmitoyltransferase, methyltransferase, demethylase, acetyltransferase, deacetylase, glycosyltransferase, fp53E3 ligase, SUMO ligase, ubiquitin ligase, deubiquitinase, tyrosine sulfotransferase, heat shock protein, bromodomain, Tudor domain, PWWP domain, chromodomain, ankyrin repeat, 14-3-3 protein, BRCT domain, DNA recognition protein, DNA modifying protein, or nucleus localization protein.

8. The compound of claim 1, wherein the modifier protein comprises 14-3-3σ, 14-3-3γ, 14-3-3ε, ABL, AEP1, AhR, ALK, AMPK, AR, ATAD2, ATAD2B, BAZ1A, BAZ1B, BAZ2A, BAZ2B, BCR-ABL, BRAF, BRD2, BRD3, BRD4, BRD7, BRD9, BRDT, BRFA, BRPF1A, BRPF1B, BTK, BRWD3, CBP, CREBBP, CDK2, CDK4, CDK6, CDK7, CDK9, CDK12, CECR2, cIAP, CK1, CRBN, CSF1R, CSN, DCAF11, DCAF15, DCAF16, DOT1, EGFR, ER, EZH2, FAK, FALZ, FEM1B, FKBP12, FLT3, FUT8, G9a, GCN5, GKC, GLP, G9a, HDAC1, HDAC10, HDAC11, HDAC2, HDAC3, HDAC6, HDAC8, HER2, HER4, HSC70, HSP70, HSP90, IGF2R, JAK1, JAK2, JAK3, KAT6A, KAT6B, KAT7, KDM1, KDM2, KDM4, KDM5, KDM6, KEAP1, KIT, KRAS, L3MBTL3, LRRK2, LSD1, LYN, LXR, MAPK, MAX, MEK, MET, MDM2, MLL, MOZ, mTOR, MYC, NBR1, NMT1, NSD2, NSD3, NTRK1, NTRK2, NTRK3, OTUB1, p110, p300, PAX3-FOXO1, PBRM1, PB1, PCAF, PDK1, PDK2, PDGFR, PHID, PHF1, PHF19, PIGK, PI3K, PKC, PKG, PKM2, PKR, PP2A, PP2B, PPP1R15A, PRMT1, PRMT3, PRMT4, PRMT5, PRMT6, PTP1B, Raf-1, RET, RNF4, RNF114, ROCK, ROS1, RPN11, SETD2, SFN, SHP1, SHP2, SIRT1, SIRT3, SIRT6, SMARCA2, SMSARCA4, SMYD2, SMYD3, SOS1, SRC, ST6GAL1, SYK, TAF1, TDRD, Tie2, TOP1, TPST1, TRAF6, TRIM22, TRIM24, TRIM33, TRIM33B, TRIM66, TrkB, TYK2, UAF1, UCHL1, ULK1, USP1, USP7, USP8, USP9X, USP14, USP30, VEGFR1, VEGFR2, VEGFR3, VE-PTP, VHL, WEE1, WRD9/BRWD1, XIAP, YWHAE, YWHAG, ZDHHC11, ZDHHC21, ZDHHC3, or ZUP1, or ZMYND8, ZMYND11.

9. The compound of claim 1, wherein the linker comprises: (a) a structure selected from the group consisting of polyethylene glycol, an aromatic group, an alkyl, an alkenyl, an alkyl phosphate, an alkyl siloxane, an epoxy, a glycidyl, cycloalkane, heterocycloalkane, a carboxylate, an anhydride, a piperazine, a piperidyl, and a triazole; or (b) a polypeptide of natural or synthetic source having a chain length of between 2 to 24 atoms.

10. The compound of claim 1, wherein the compound has a structure of Formula I: 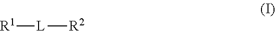
wherein:
R¹ is a targeting ligand selected from the group consisting of: 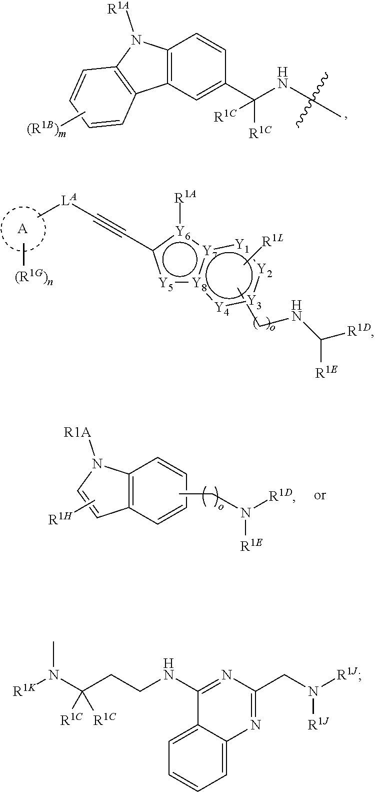
L is a linker selected from the group consisting of: polyethylene glycol (PEG), C₁₋₅₀ alkylene-PEG, C₂₋₅₀ alkenylene-PEG, C₂₋₅₀ alkynylene-PEG, C₁₋₅₀ alkylene, C₂₋₅₀ alkenylene, C₂₋₅₀ alkynylene, C₃₋₁₂ cycloalkyl, 3- to 12-membered heterocycloalkyl, 3- to 12-membered heterocycloalkyl-C₁₋₁₀ alkylene, 3- to 12-membered heterocycloalkyl-PEG, or PEG-3- to 12-membered heterocycloalkyl-PEG, wherein the PEG or C₁₋₅₀ alkylene are optionally substituted with halogen or C₆₋₁₂ aryl;
R² is a recruiting ligand;
Ring A is C₃₋₁₂ cycloalkyl, 3- to 12-membered heterocycloalkyl, C₆₋₁₂ aryl or 3- to 12-membered heteroaryl;
Y¹, Y², Y³, Y⁴, Y⁵, Y⁶, Y⁷, and Y⁸ are each independently a bond, CH, or N;
R^{1A} is a cysteine reactive group, C₁₋₆ alkyl, C₃₋₁₂ cycloalkyl, 3- to 12-membered heterocycloalkyl, —SOR^{1A}, or —COR^{1A1}, wherein the C₁₋₆ alkyl and C₃₋₁₂ cycloalkyl is optionally substituted with —CN, halogen, or C₁₋₆alkylamines;
R^{1A} is C₁₋₆ alkyl, C₂₋₆ alkenyl, or C₂₋₆ alkynyl, wherein the C₁₋₆ alkyl, C₂₋₆ alkenyl, and C₂₋₆ alkynyl are optionally substituted with —CN, halogen, C₁₋₆ alkyl, or C₁₋₆ alkylamines;
each R^{1B} is a C₆₋₁₂ aryl or 3- to 12-membered heteroaryl, wherein the C₆₋₁₂ aryl and 3- to 12-membered heteroaryl are optionally substituted with C₁₋₆ alkyl;
each R^{1C} is independently hydrogen, or two Ric taken together form an oxo;
R^{1D} is hydrogen or C₁₋₆ alkyl;
R^{1E} is C₁₋₆ alkyl, C₃₋₂ cycloalkyl, 3- to 12-membered heterocycloalkyl, C₆₋₁₂ aryl, or 3- to 12-membered heteroaryl, wherein the C₁₋₆ alkyl, C₃₋₁₂ cycloalkyl, 3- to 12-membered heterocycloalkyl, C₆₋₁₂ aryl, and 3- to 12-membered heteroaryl are optionally substituted with halogen, C₁₋₆ alkyl, C₁₋₆ alkoxy, C₁₋₆ carboxylic acid, —NH₂, or —N(C₁₋₆alkyl)₂; or
R^{1D} and R^{1E} are combined to form a C₃₋₁₂ cycloalkyl, 3- to 12-membered heterocycloalkyl, C₆₋₁₂ aryl or 3- to 12-membered heteroaryl, wherein the C₃₋₁₂ cycloalkyl, 3- to 12-membered heterocycloalkyl, C₆₋₁₂ aryl or 3- to 12-membered heteroaryl are optionally substituted with deuterium, halogen, —OH, —OC₁₋₆ alkyl, C₁₋₆ alkyl, C₁₋₆ alkoxy, C₁₋₆ carboxylic acid, —NH₂, or —N(C₁₋₆alkyl)₂;
R^{1F} is hydrogen or C₁₋₆ alkyl;
each R^{1G} is independently deuterium, —CN, halogen, C₁₋₆ alkyl, C₁₋₆ alkoxy, C₁₋₆ haloalkyl, —COOR^{1GB}, —CONR^{1GA}R^{1GB}, —NO₂, —NR^{1B}SO₂, —SO₂(C₁₋₆alkyl), —SO₂NR^{1GA}R^{1GB}, —SO₂NH-heteroaryl, or two R^{1G} taken together form an aryl or heteroaryl ring;
R^{1GA} is hydrogen or C₁₋₆ alkyl;
R^{1GB} is hydrogen, —COC₁₋₆ alkyl, C₁₋₆alkyl, C₆₋₁₂ aryl, 3- to 12-membered heteroaryl;
R^{1H} is a C₆₋₁₂ aryl or 3- to 12-membered heteroaryl, wherein the C₆₋₁₂ aryl and 3- to 12-membered heteroaryl are optionally substituted with halogen, C₁₋₆ alkyl, or C₁₋₆ alkylene-NH(C₁₋₆alkyl);
each R^{1J} is independently C₁₋₆ alkyl or C₃₋₁₂ cycloalkyl, wherein the C₁₋₆ alkyl and C₃₋₁₂ cycloalkyl are optionally substituted with halogen or C₁₋₆ alkyl; or both R^{1J} is combined to form a 3- to 12-membered heteroalkyl optionally substituted with R^{1JA};
R^{1JA} is diphenyl methyl or C₁₋₆ alkylene-N(R^{1JA1})(R^{1JA2}), wherein the diphenyl methyl is optionally substituted with halogen;
each of R^{1JA1} and R^{1JA2} is independently C₁₋₆ alkyl or C₃₋₁₂ cycloalkyl, wherein the C₃₋₁₂ cycloalkyl is optionally substituted with C₁₋₆ alkyl;
R^{1K} is a C₁₋₆ alkyl;
R^{1L} is deuterium, halogen, —OH, —OC₁₋₆ alkyl, —OC₁₋₆ haloalkyl, —NH₂, NHC₁₋₆ alkyl, —N(C₁₋₆ alkyl)₂, —NO₂, —NHCOMe, —OCOMe;
L^{A} is optionally substituted C₁₋₃ alkyl, —NH—, —NC₁₋₃ alkyl, —O—, —S—, —SO—, —SO₂—, or a combination thereof, wherein the C₁₋₃ alkyl is optionally substituted with one or more deuterium or halogen;
m is an integer between 0 and 4;
n is an integer between 0 to 10;
o is 0 or 1;
wherein the functional group of R^{1E} or one R^{1G} is the point of attachment to linker L for the following structure: 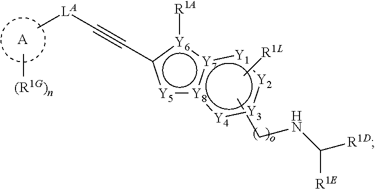
wherein the functional group of R^{1E} or R^{1H} is the point of attachment to linker L for the following structure: 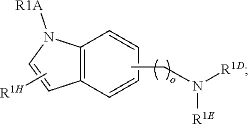
and
wherein the functional group of R^{1J} or R^{1K} is the point of attachment to linker L for the following structure:

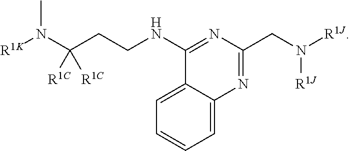

11. (canceled)

12. The compound of claim 10, wherein R¹ is a compound of Formula (II): 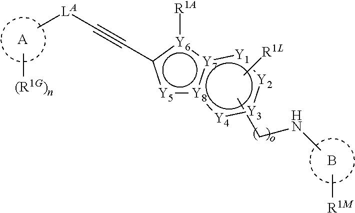
wherein:
Ring B is C₃₋₁₂ cycloalkyl or 3- to 12-membered heterocycloalkyl;
R^{1M} is deuterium, halogen, or —OR^{1MA}; and
R^{1MA} is C₁₋₆ alkyl or C₁₋₆ alkoxy.

13. The compound of claim 10, wherein R^{1L} is deuterium, —F, —Cl, —Br, —I, —OH, —OMe, —OCF₃, —NHMe, —NMe₂, —NO₂, —NHCOMe, or —OCOMe.

14. The compound of claim 12, wherein Ring A-L^{A} structure has the following structure: 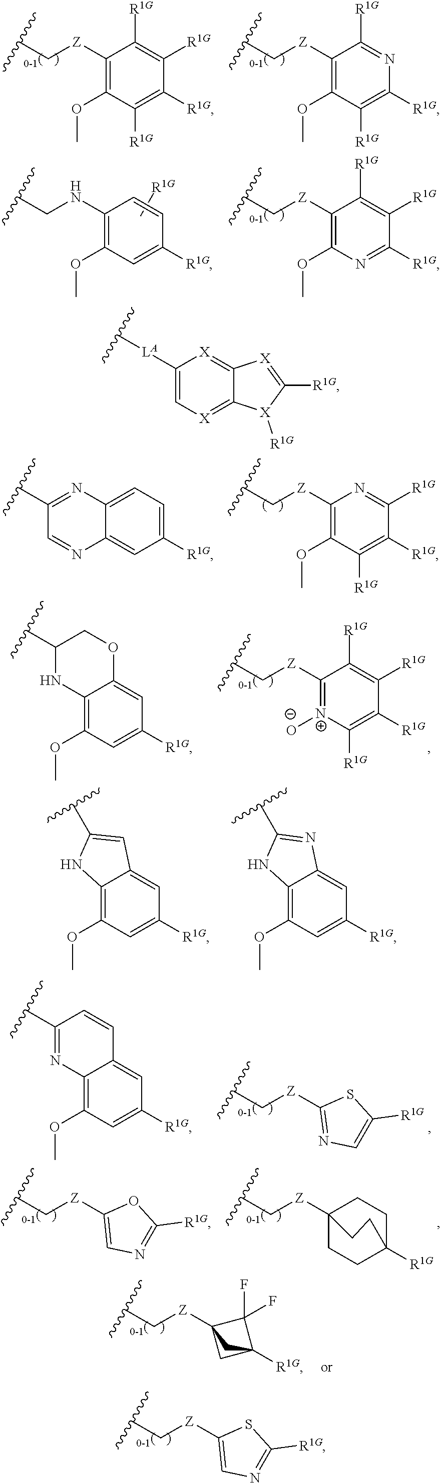
wherein:
X is N, O, S, or CH; and
Z is —NH—, —N(C₁₋₆ alkyl)-, —O—, —S—, —SO—, —SO₂—.

15. (canceled)

16. (canceled)

17. The compound of claim 10, wherein R² is:

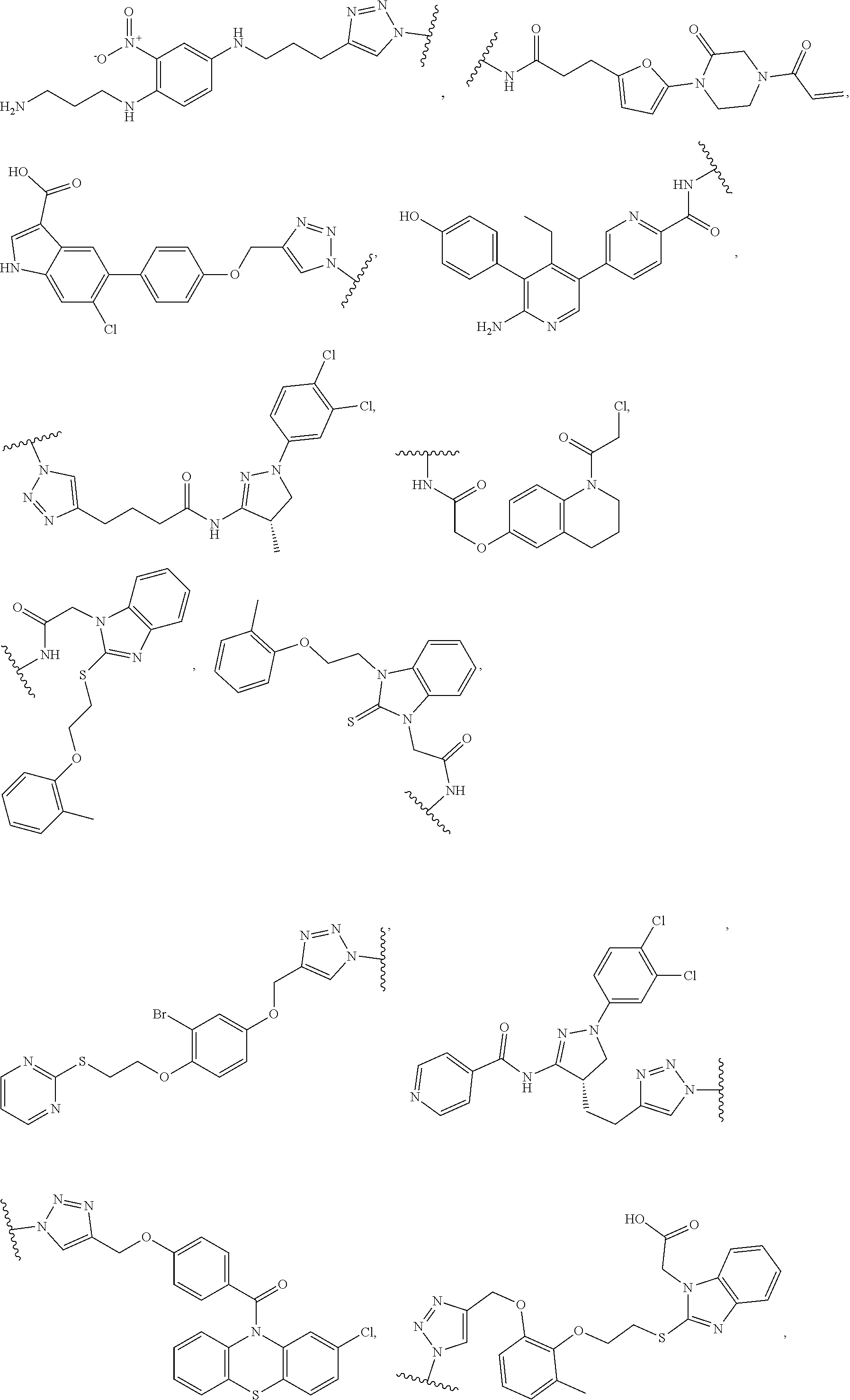

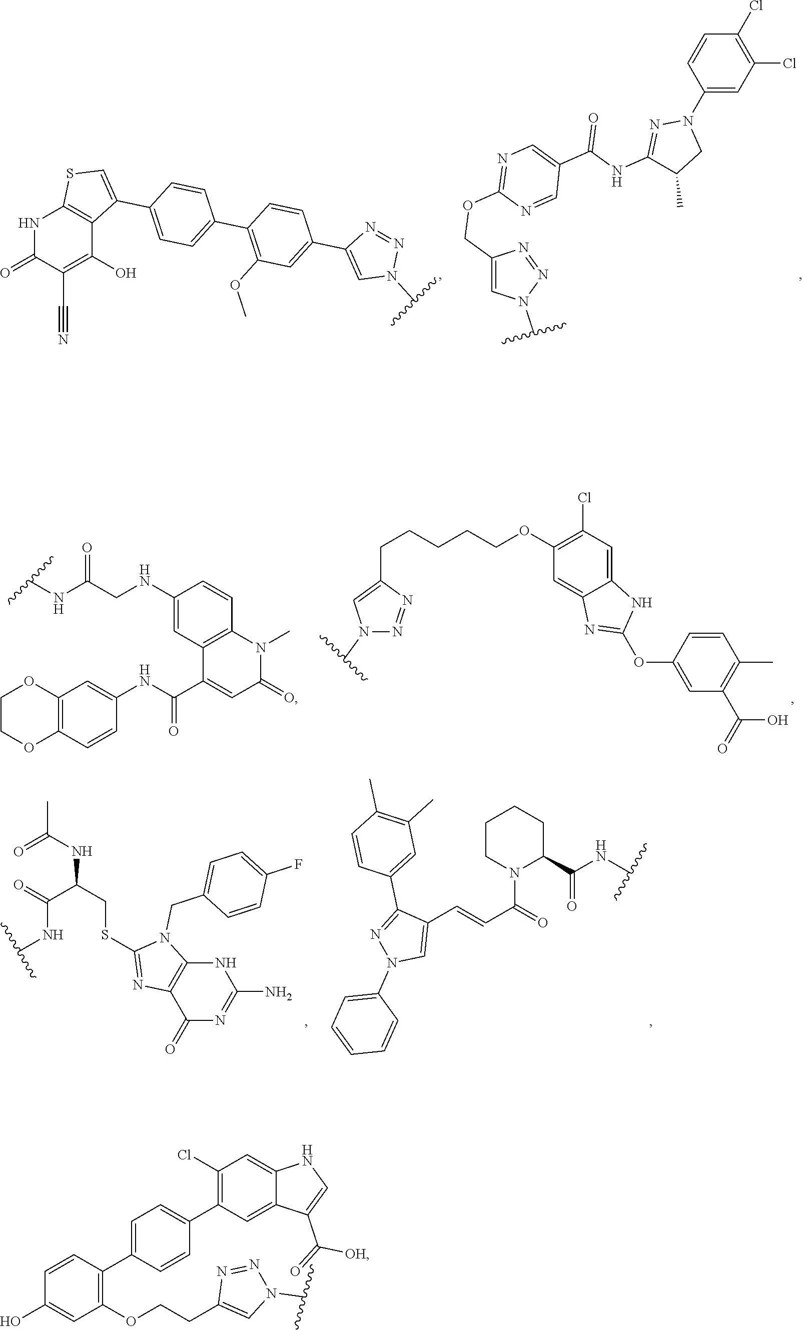

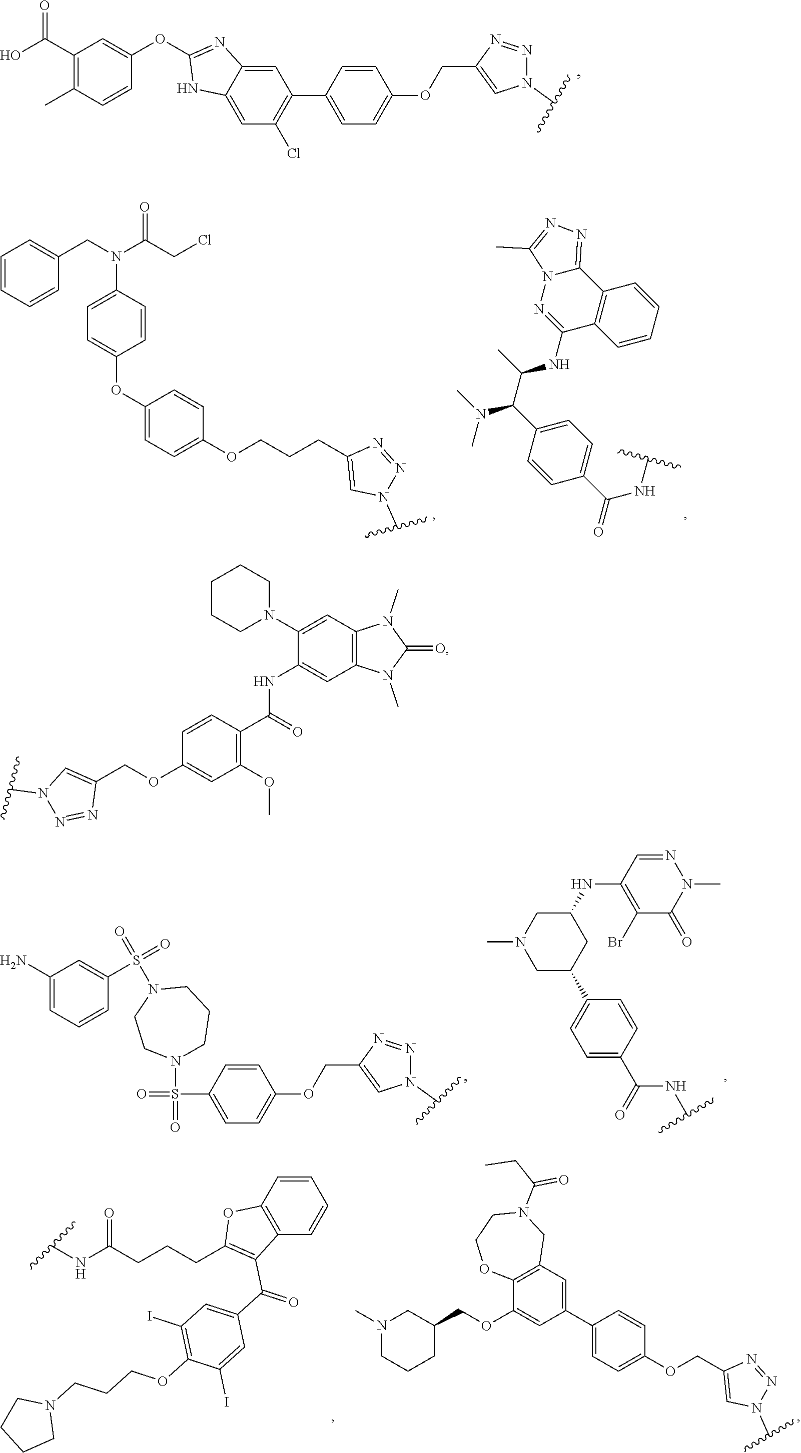

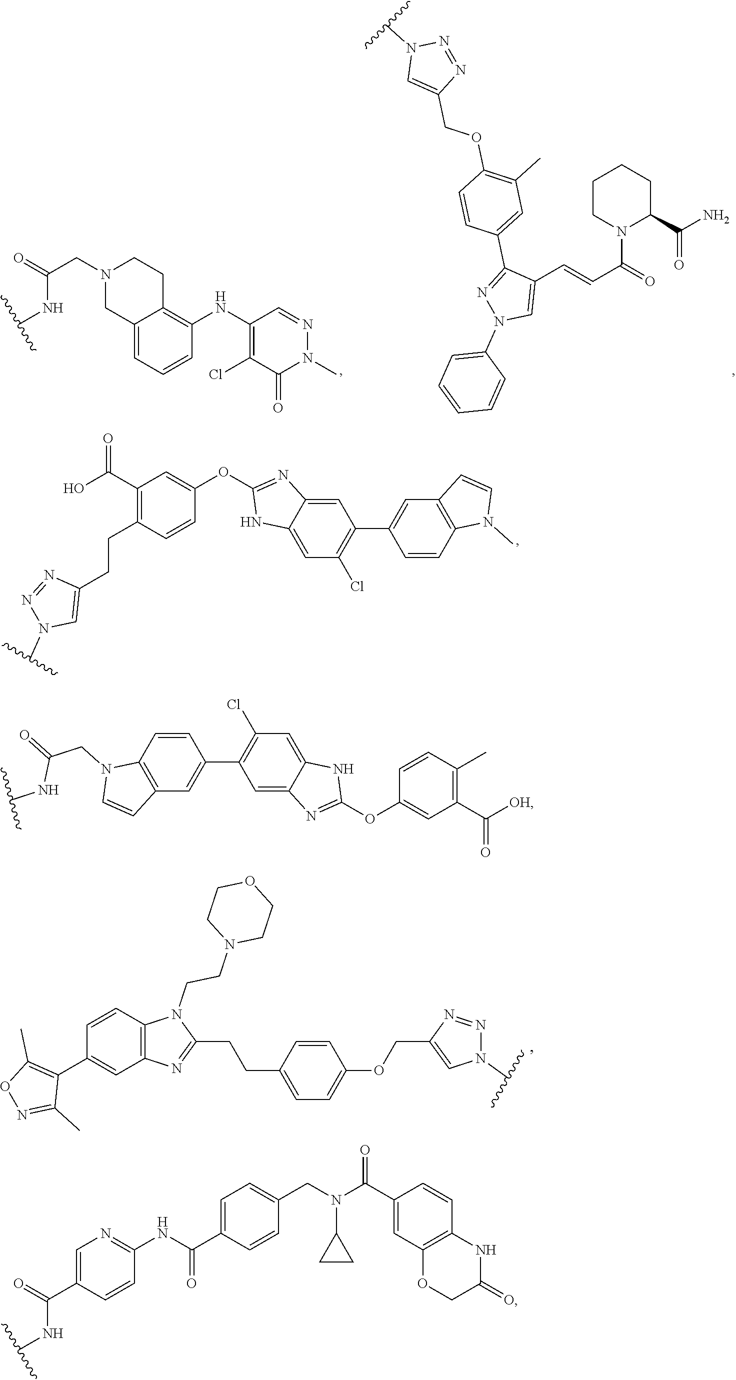 [SMILES: *NC(=O)CN1CCc2c(cccc2Nc2cnn(C)c(=O)c2Cl)C1 ; *n1cc(COc2ccc(-c3nn(-c4ccccc4)cc3/C=C/C(=O)N3CCCC[C@H]3C(N)=O)cc2C)nn1 ; *n1cc(CCc2ccc(Oc3nc4cc(-c5ccc6c(ccn6C)c5)c(Cl)cc4[nH]3)cc2C(=O)O)nn1 ; *NC(=O)Cn1ccc2cc(-c3cc4nc(Oc5ccc(C)c(C(=O)O)c5)[nH]c4cc3Cl)ccc21 ; *n1cc(COc2ccc(CCc3nc4cc(-c5c(C)noc5C)ccc4n3CCN3CCOCC3)cc2)nn1 ; *NC(=O)c1ccc(NC(=O)c2ccc(CN(C(=O)c3ccc4c(c3)OCC(=O)N4)C3CC3)cc2)nc1]

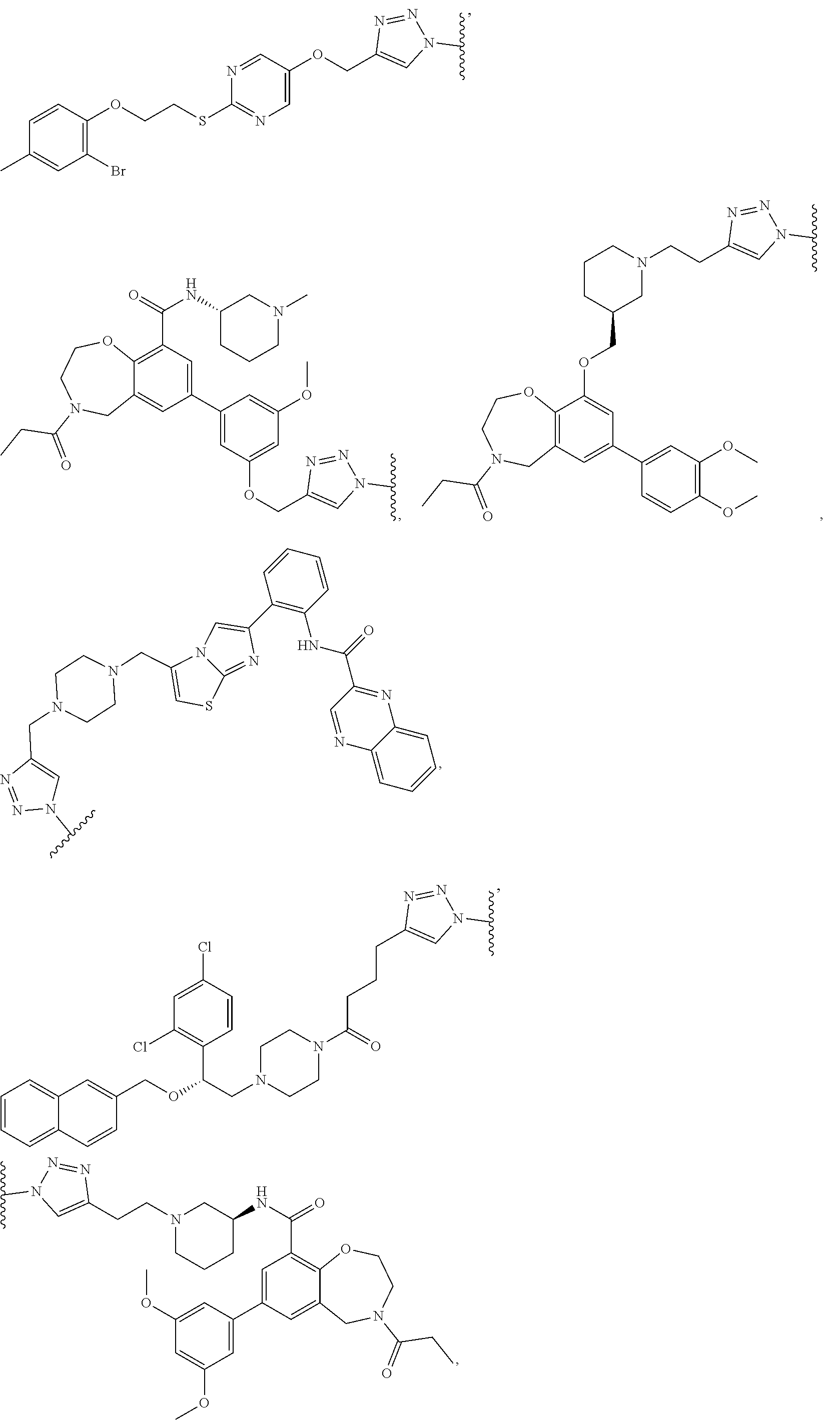

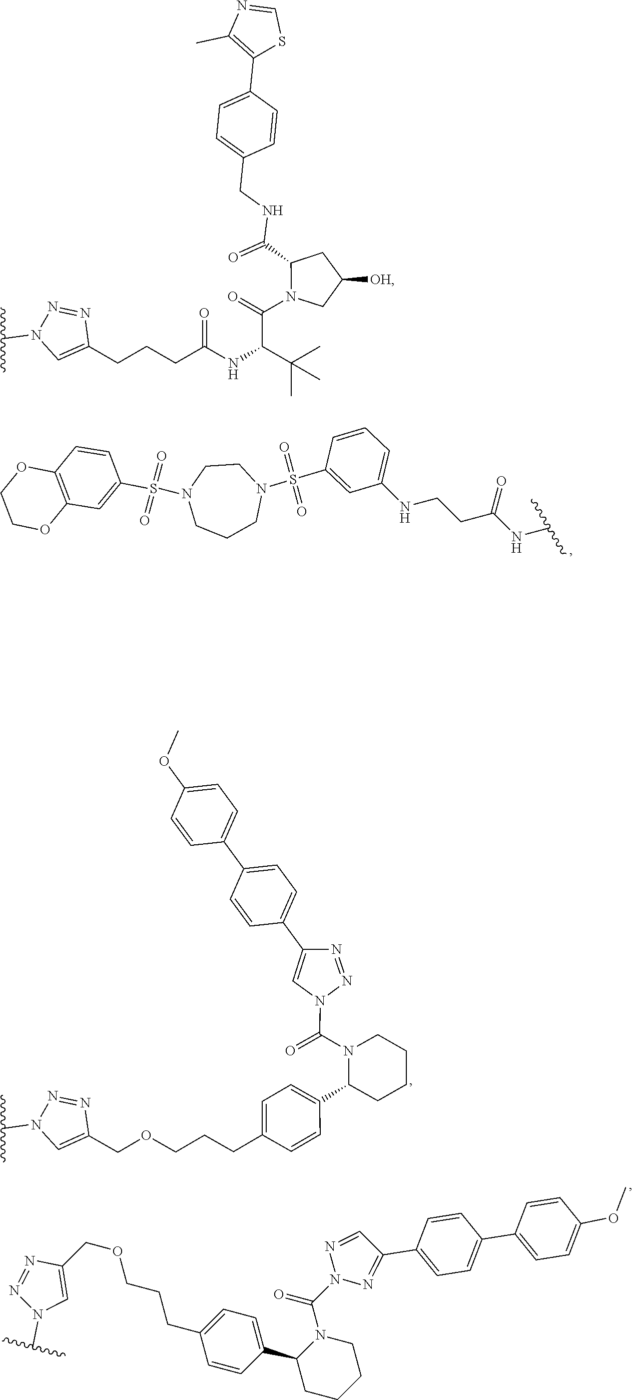 [SMILES: *n1cc(CCCC(=O)N[C@H](C(=O)N2C[C@H](O)C[C@H]2C(=O)NCc2ccc(-c3scnc3C)cc2)C(C)(C)C)nn1 ; *NC(=O)CCNc1cccc(S(=O)(=O)N2CCCN(S(=O)(=O)c3ccc4c(c3)OCCO4)CC2)c1 ; *n1cc(COCCCc2ccc([C@H]3CCCCN3C(=O)n3cc(-c4ccc(-c5ccc(OC)cc5)cc4)nn3)cc2)nn1 ; *n1cc(COCCCc2ccc([C@@H]3CCCCN3C(=O)n3ncc(-c4ccc(-c5ccc(OC)cc5)cc4)n3)cc2)nn1]

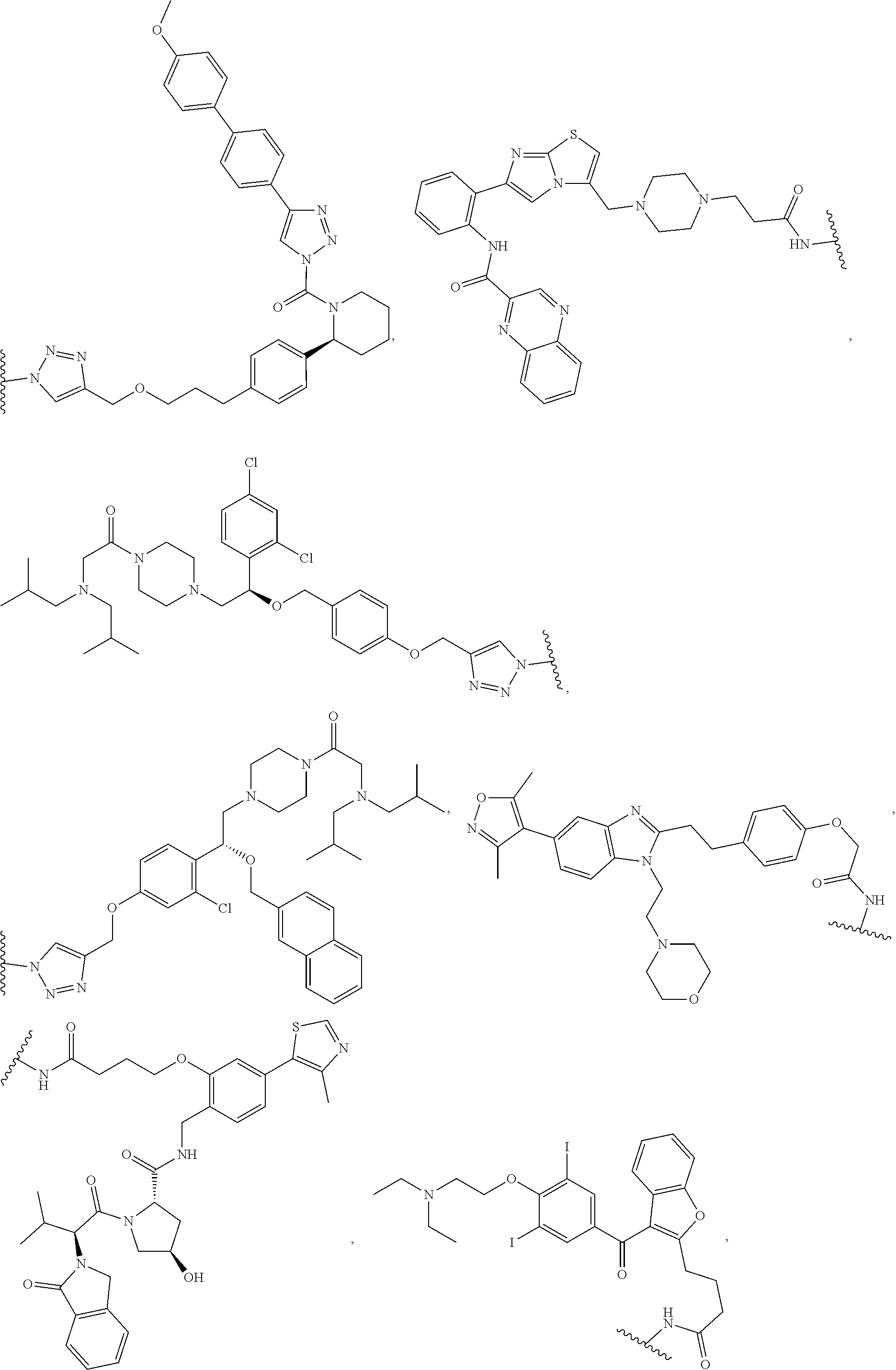

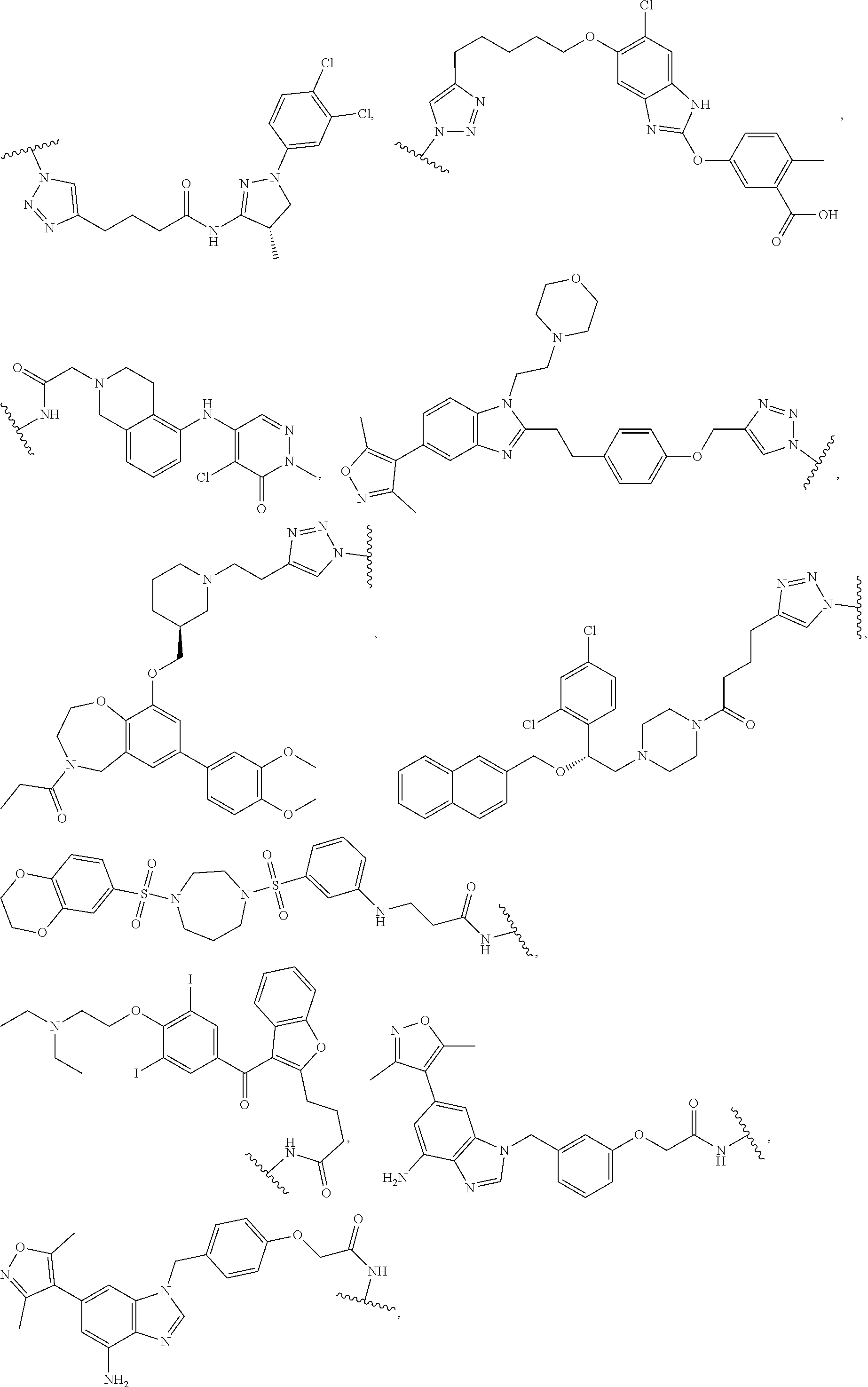

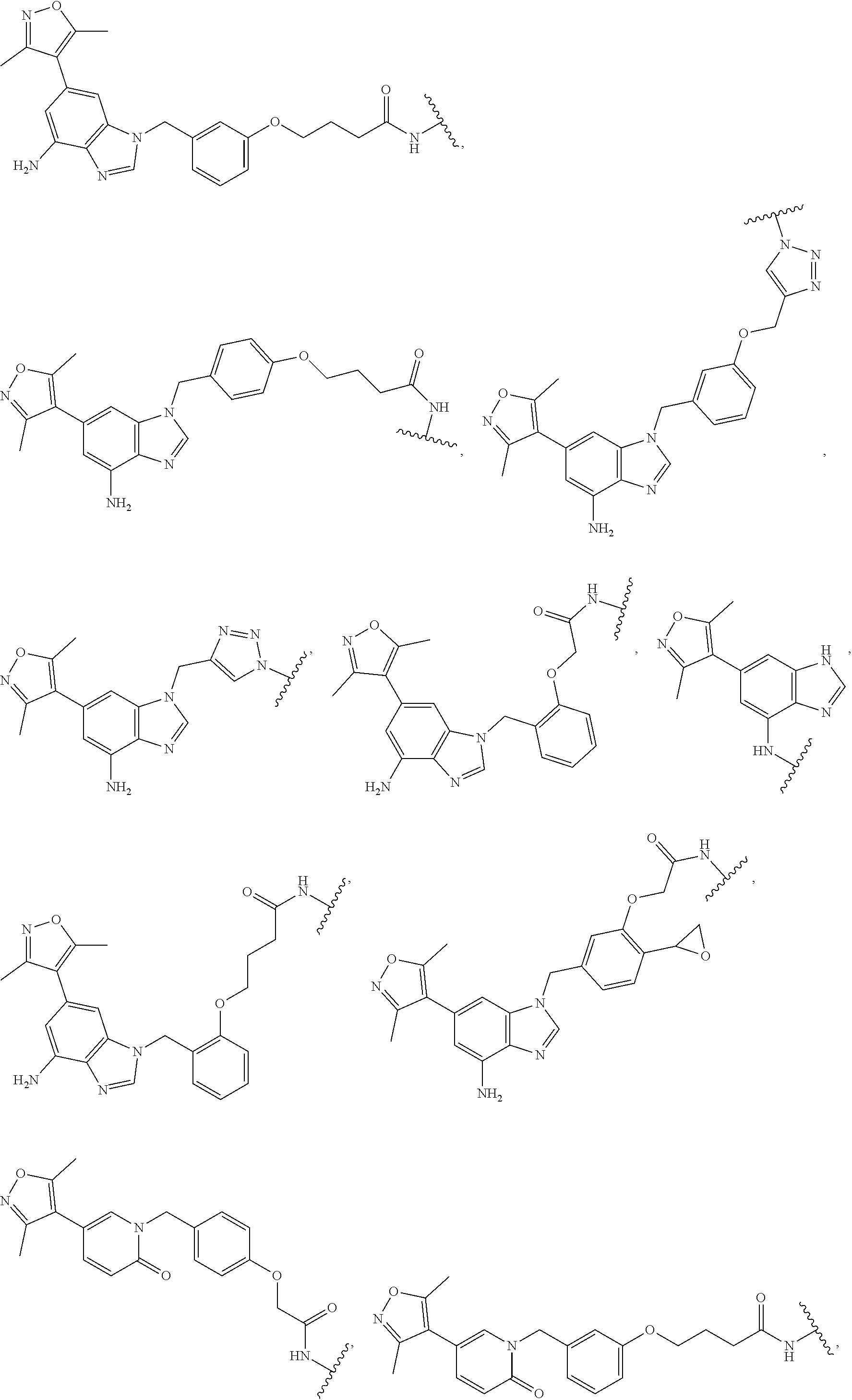

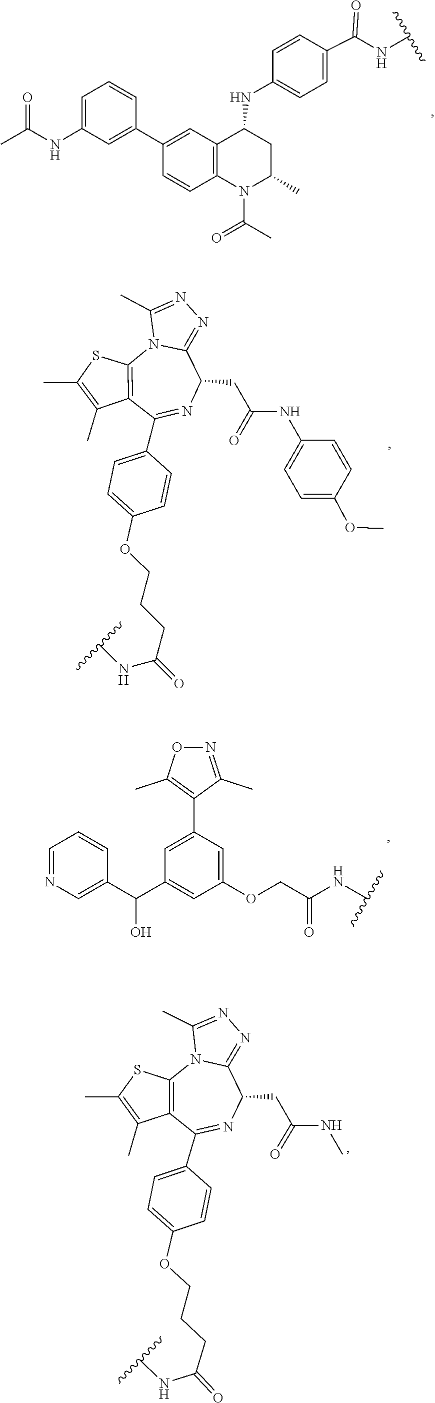

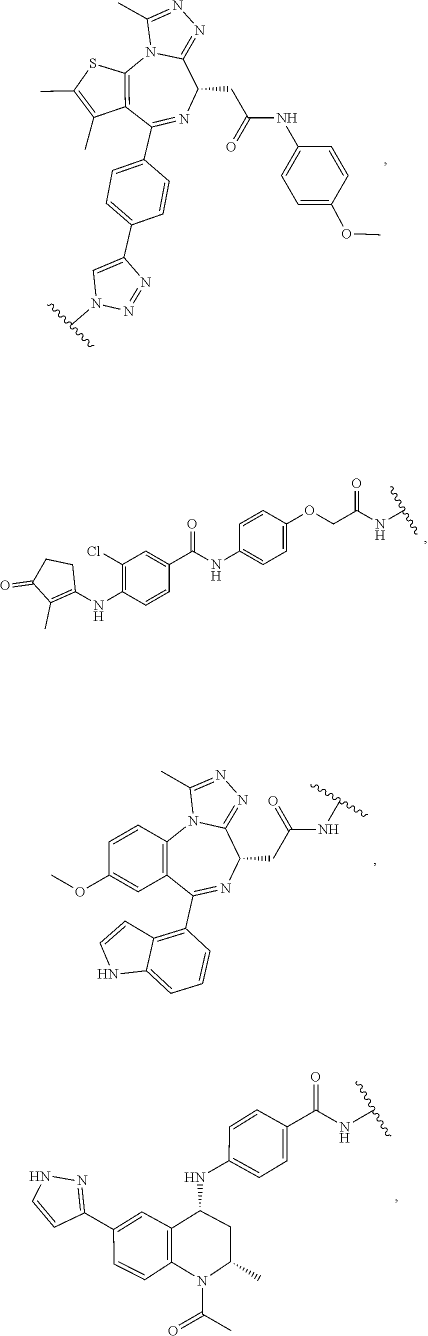

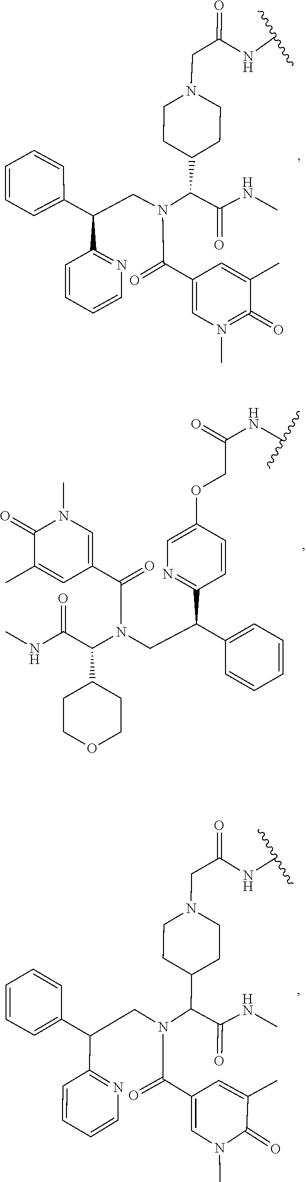

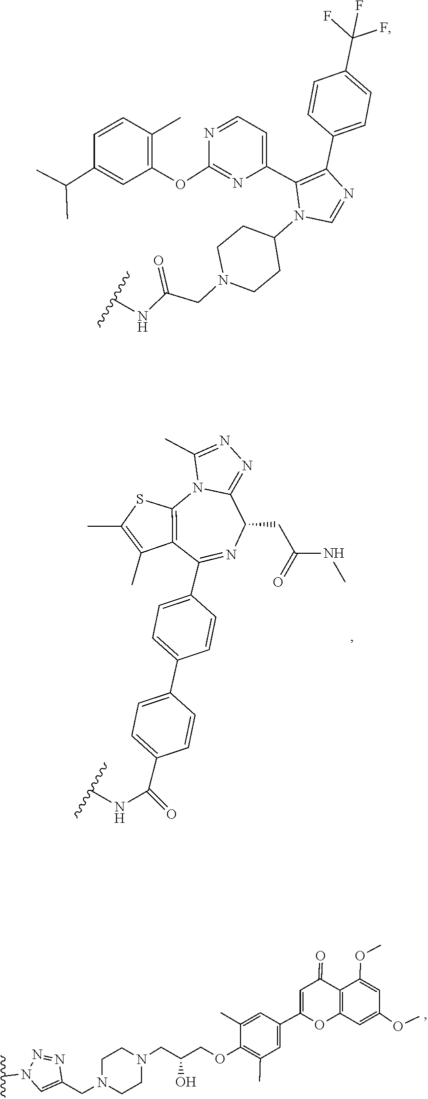

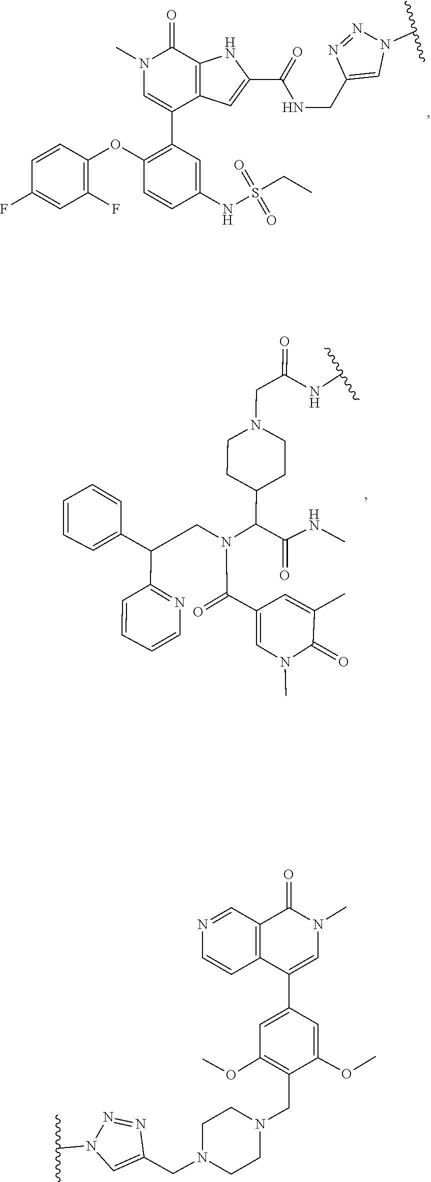 [SMILES: *n1cc(CNC(=O)c2cc3c(-c4cc(NS(=O)(=O)CC)ccc4Oc4ccc(F)cc4F)cn(C)c(=O)c3[nH]2)nn1 ; *NC(=O)CN1CCC(C(C(=O)NC)N(CC(c2ccccc2)c2ccccn2)C(=O)c2cc(C)c(=O)n(C)c2)CC1 ; *n1cc(CN2CCN(Cc3c(OC)cc(-c4cn(C)c(=O)c5cnccc45)cc3OC)CC2)nn1]

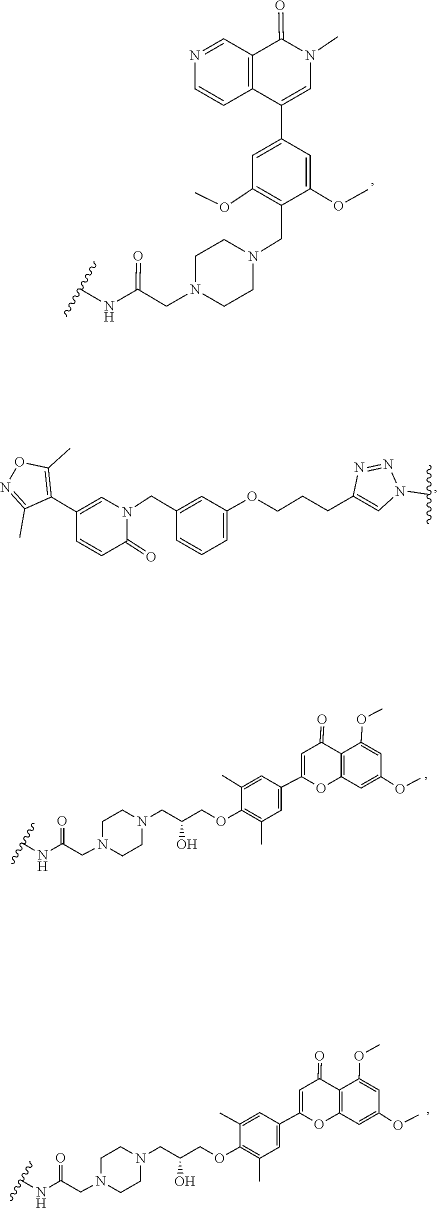 [SMILES: *NC(=O)CN1CCN(Cc2c(OC)cc(-c3cn(C)c(=O)c4cnccc34)cc2OC)CC1 ; *n1cc(CCCOc2cccc(Cn3cc(-c4c(C)noc4C)ccc3=O)c2)nn1 ; *NC(=O)CN1CCN(C[C@@H](O)COc2c(C)cc(-c3cc(=O)c4c(OC)cc(OC)cc4o3)cc2C)CC1 ; *NC(=O)CN1CCN(C[C@@H](O)COc2c(C)cc(-c3cc(=O)c4c(OC)cc(OC)cc4o3)cc2C)CC1]

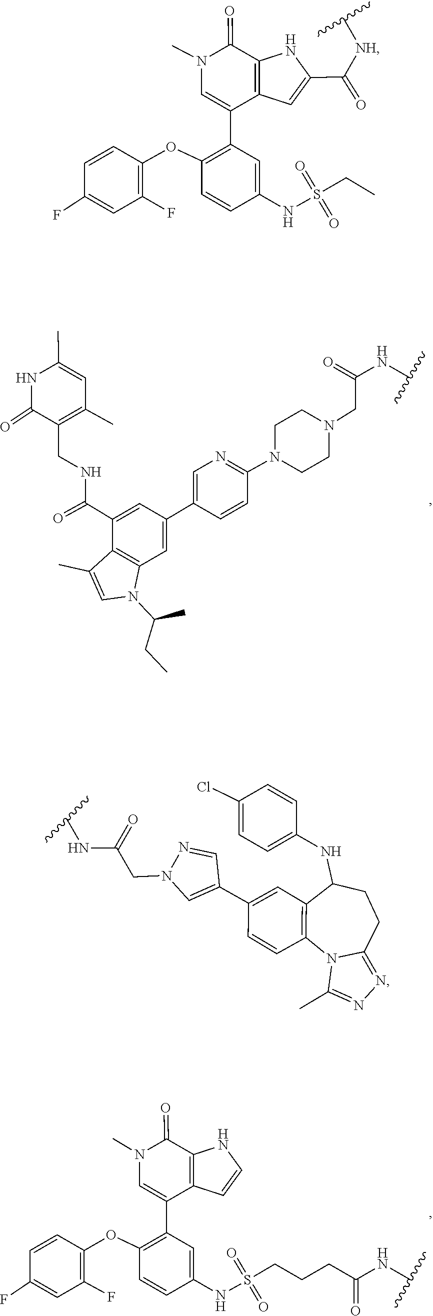 [SMILES: *NC(=O)c1cc2c(-c3cc(NS(=O)(=O)CC)ccc3Oc3ccc(F)cc3F)cn(C)c(=O)c2[nH]1 ; *NC(=O)CN1CCN(c2ccc(-c3cc(C(=O)NCc4c(C)cc(C)[nH]c4=O)c4c(C)cn([C@@H](C)CC)c4c3)cn2)CC1 ; *NC(=O)Cn1cc(-c2ccc3c(c2)C(Nc2ccc(Cl)cc2)CCc2nnc(C)n2-3)cn1 ; *NC(=O)CCCS(=O)(=O)Nc1ccc(Oc2ccc(F)cc2F)c(-c2cn(C)c(=O)c3[nH]ccc23)c1]

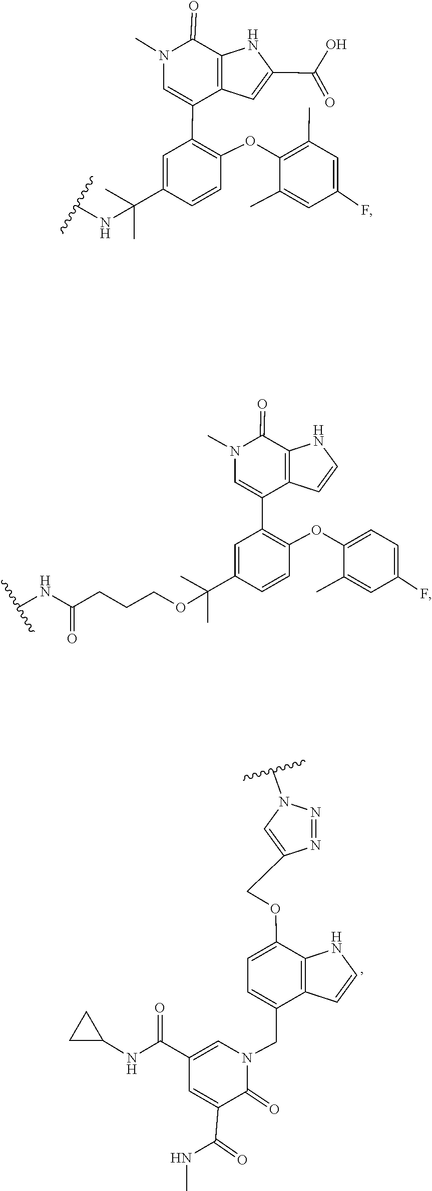 [SMILES: *NC(C)(C)c1ccc(Oc2c(C)cc(F)cc2C)c(-c2cn(C)c(=O)c3[nH]c(C(=O)O)cc23)c1 ; *NC(=O)CCCOC(C)(C)c1ccc(Oc2ccc(F)cc2C)c(-c2cn(C)c(=O)c3[nH]ccc23)c1 ; *n1cc(COc2ccc(Cn3cc(C(=O)NC4CC4)cc(C(=O)NC)c3=O)c3cc[nH]c23)nn1]

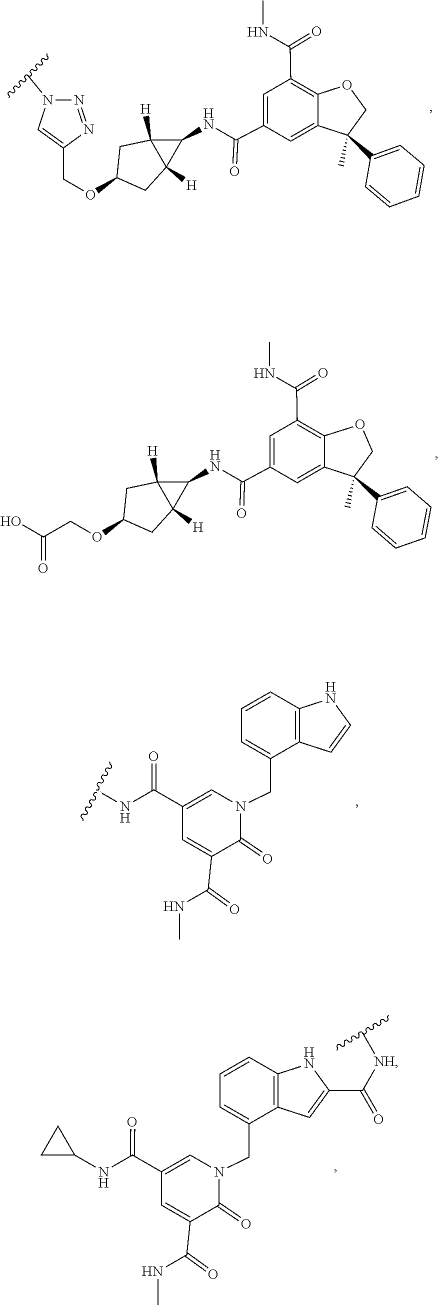

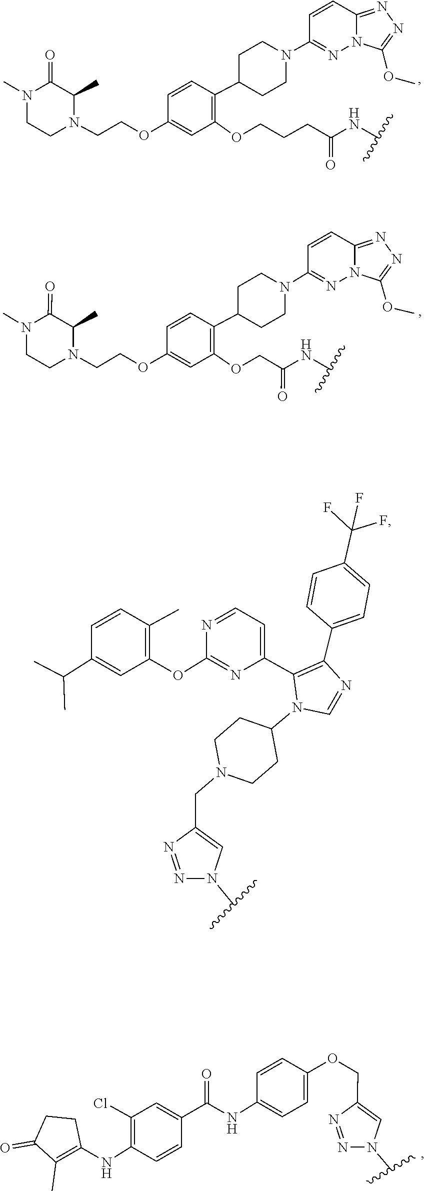

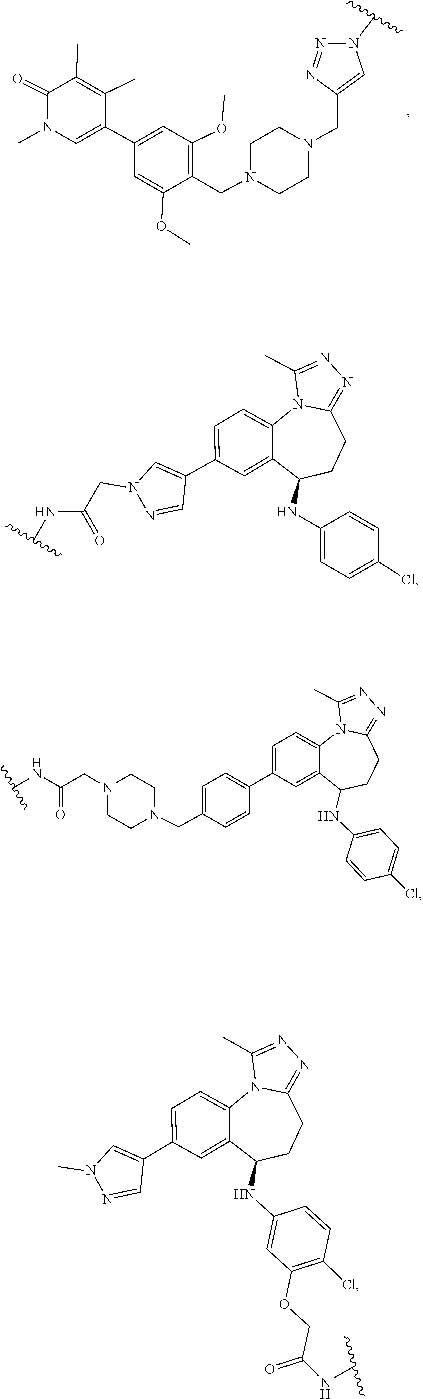 [SMILES: *n1cc(CN2CCN(Cc3c(OC)cc(-c4cn(C)c(=O)c(C)c4C)cc3OC)CC2)nn1 ; *NC(=O)Cn1cc(-c2ccc3c(c2)[C@H](Nc2ccc(Cl)cc2)CCc2nnc(C)n2-3)cn1 ; *NC(=O)CN1CCN(Cc2ccc(-c3ccc4c(c3)C(Nc3ccc(Cl)cc3)CCc3nnc(C)n3-4)cc2)CC1 ; *NC(=O)COc1cc(N[C@@H]2CCc3nnc(C)n3-c3ccc(-c4cnn(C)c4)cc32)ccc1Cl]

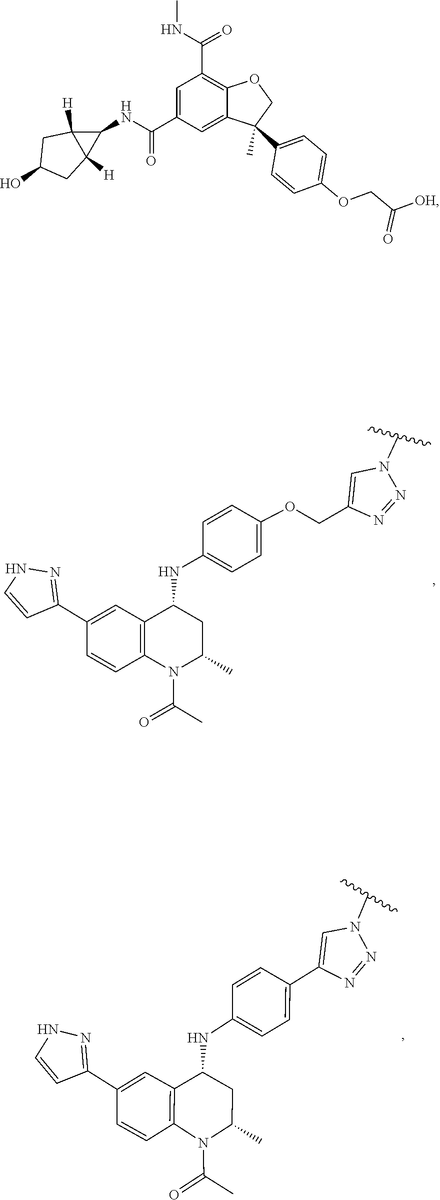 [SMILES: CNC(=O)c1cc(C(=O)N[C@H]2[C@@H]3C[C@@H](O)C[C@@H]32)cc2c1OC[C@@]2(C)c1ccc(OCC(=O)O)cc1 ; *n1cc(COc2ccc(N[C@@H]3C[C@H](C)N(C(C)=O)c4ccc(-c5cc[nH]n5)cc43)cc2)nn1 ; *n1cc(-c2ccc(N[C@@H]3C[C@H](C)N(C(C)=O)c4ccc(-c5cc[nH]n5)cc43)cc2)nn1]

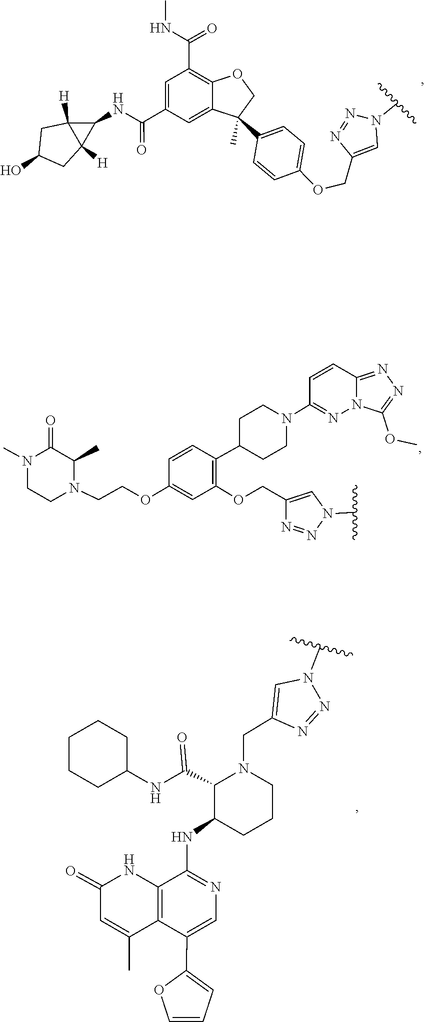

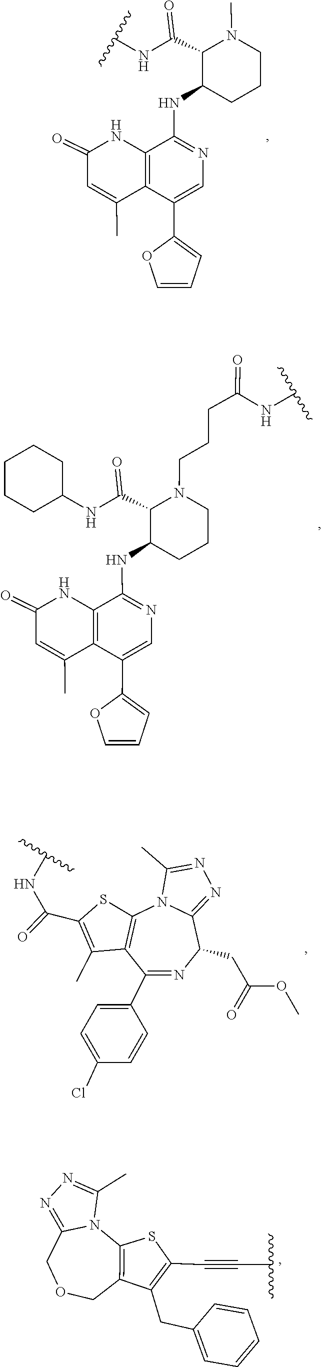 [SMILES: *NC(=O)[C@H]1[C@H](Nc2ncc(-c3ccco3)c3c(C)cc(=O)[nH]c23)CCCN1C ; *NC(=O)CCCN1CCC[C@@H](Nc2ncc(-c3ccco3)c3c(C)cc(=O)[nH]c23)[C@@H]1C(=O)NC1CCCCC1 ; *NC(=O)c1sc2c(c1C)C(c1ccc(Cl)cc1)=N[C@@H](CC(=O)OC)c1nnc(C)n1-2 ; *C#Cc1sc2c(c1Cc1ccccc1)COCc1nnc(C)n1-2]

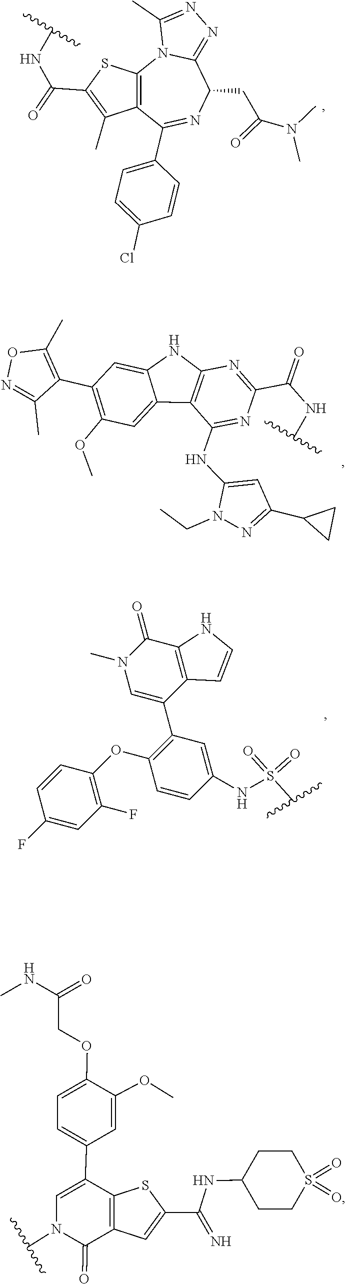

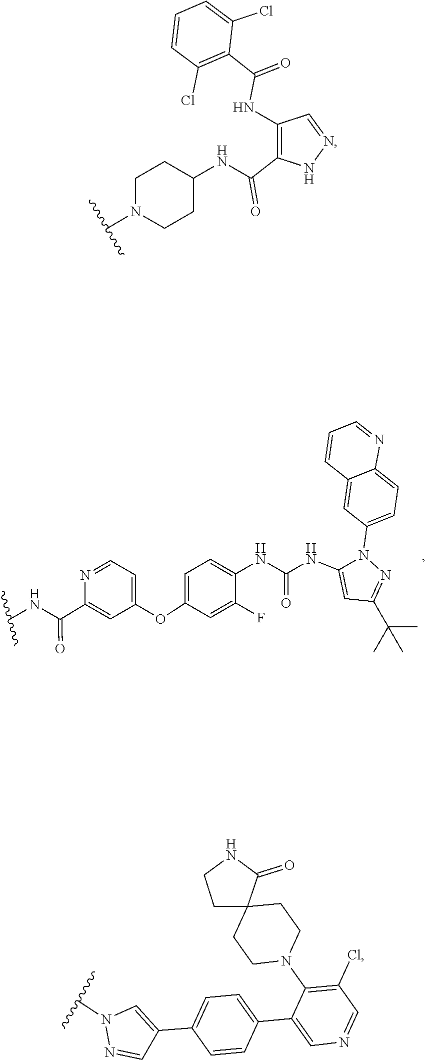 [SMILES: *N1CCC(NC(=O)c2[nH]ncc2NC(=O)c2c(Cl)cccc2Cl)CC1 ; *NC(=O)c1cc(Oc2ccc(NC(=O)Nc3cc(C(C)(C)C)nn3-c3ccc4ncccc4c3)c(F)c2)ccn1 ; *n1cc(-c2ccc(-c3cncc(Cl)c3N3CCC4(CCNC4=O)CC3)cc2)cn1]

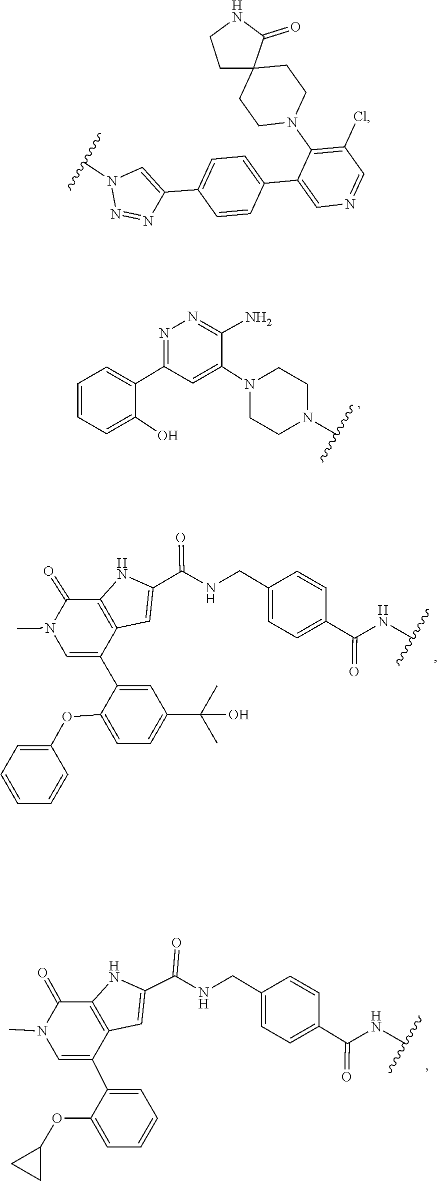 [SMILES: *n1cc(-c2ccc(-c3cncc(Cl)c3N3CCC4(CCNC4=O)CC3)cc2)nn1 ; *N1CCN(c2cc(-c3ccccc3O)nnc2N)CC1 ; *NC(=O)c1ccc(CNC(=O)c2cc3c(-c4cc(C(C)(C)O)ccc4Oc4ccccc4)cn(C)c(=O)c3[nH]2)cc1 ; *NC(=O)c1ccc(CNC(=O)c2cc3c(-c4ccccc4OC4CC4)cn(C)c(=O)c3[nH]2)cc1]

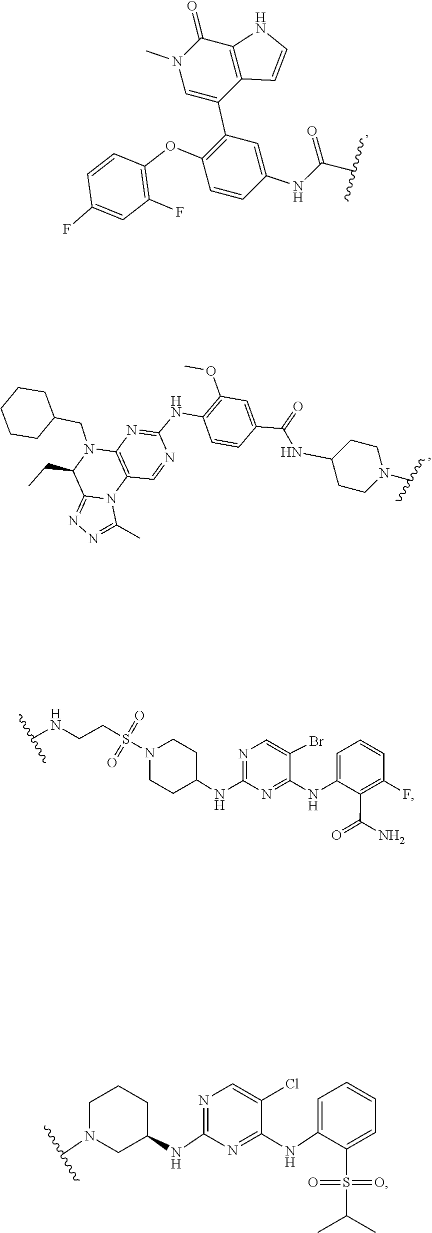 [SMILES: *C(=O)Nc1ccc(Oc2ccc(F)cc2F)c(-c2cn(C)c(=O)c3[nH]ccc23)c1 ; *N1CCC(NC(=O)c2ccc(Nc3ncc4c(n3)N(CC3CCCCC3)[C@H](CC)c3nnc(C)n3-4)c(OC)c2)CC1 ; *NCCS(=O)(=O)N1CCC(Nc2ncc(Br)c(Nc3cccc(F)c3C(N)=O)n2)CC1 ; *N1CCC[C@@H](Nc2ncc(Cl)c(Nc3ccccc3S(=O)(=O)C(C)C)n2)C1]

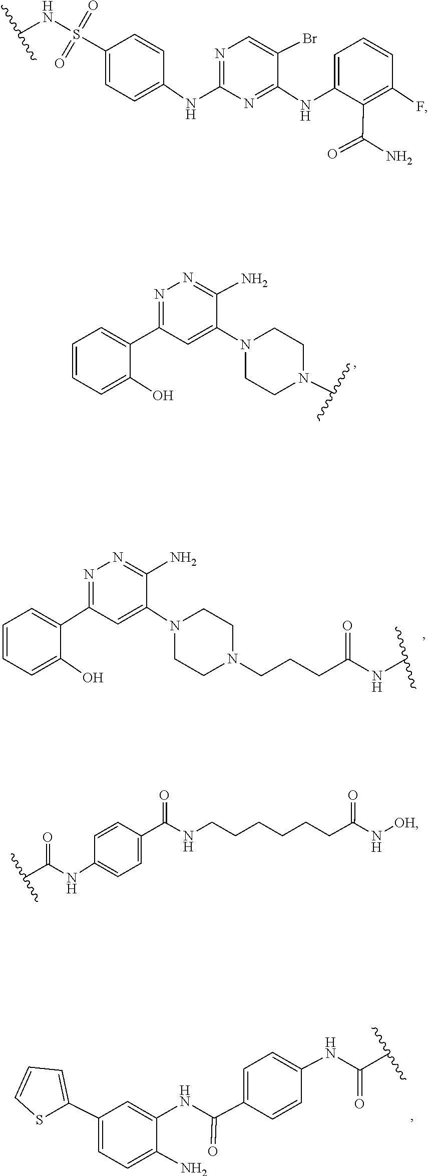 [SMILES: *NS(=O)(=O)c1ccc(Nc2ncc(Br)c(Nc3cccc(F)c3C(N)=O)n2)cc1 ; *N1CCN(c2cc(-c3ccccc3O)nnc2N)CC1 ; *NC(=O)CCCN1CCN(c2cc(-c3ccccc3O)nnc2N)CC1 ; *C(=O)Nc1ccc(C(=O)NCCCCCCC(=O)NO)cc1 ; *C(=O)Nc1ccc(C(=O)Nc2cc(-c3cccs3)ccc2N)cc1]

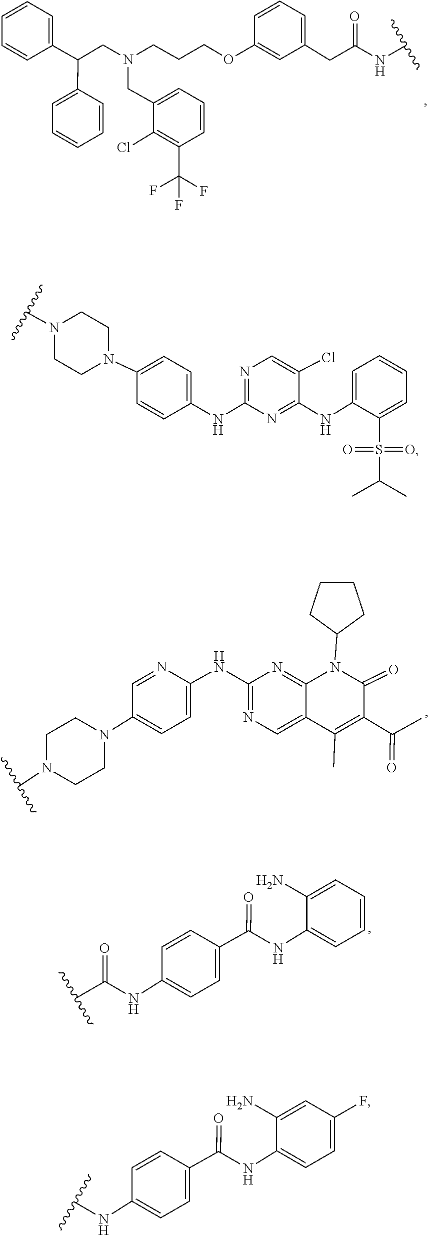 [SMILES: *NC(=O)Cc1cccc(OCCCN(Cc2cccc(C(F)(F)F)c2Cl)CC(c2ccccc2)c2ccccc2)c1 ; *N1CCN(c2ccc(Nc3ncc(Cl)c(Nc4ccccc4S(=O)(=O)C(C)C)n3)cc2)CC1 ; *N1CCN(c2ccc(Nc3ncc4c(C)c(C(C)=O)c(=O)n(C5CCCC5)c4n3)nc2)CC1 ; *C(=O)Nc1ccc(C(=O)Nc2ccccc2N)cc1 ; *Nc1ccc(C(=O)Nc2ccc(F)cc2N)cc1]

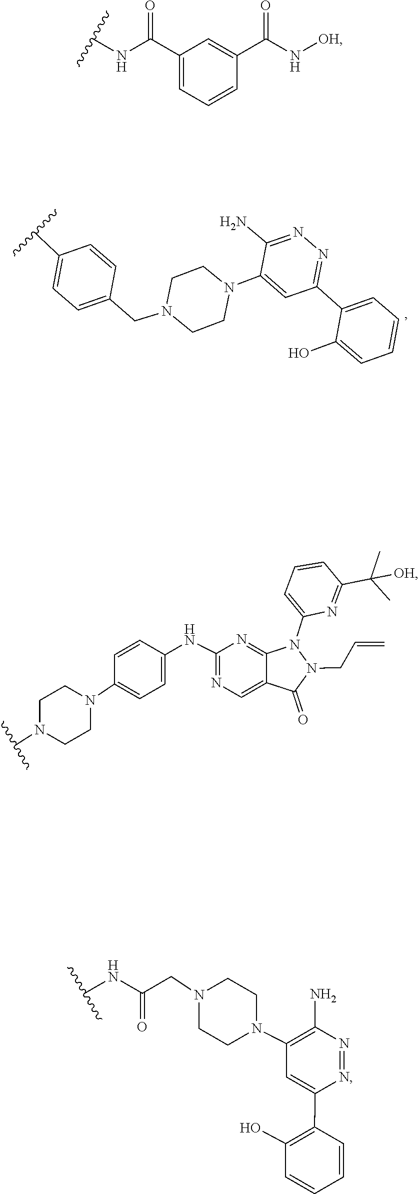 [SMILES: *NC(=O)c1cccc(C(=O)NO)c1 ; *c1ccc(CN2CCN(c3cc(-c4ccccc4O)nnc3N)CC2)cc1 ; *N1CCN(c2ccc(Nc3ncc4c(=O)n(CC=C)n(-c5cccc(C(C)(C)O)n5)c4n3)cc2)CC1 ; *NC(=O)CN1CCN(c2cc(-c3ccccc3O)nnc2N)CC1]

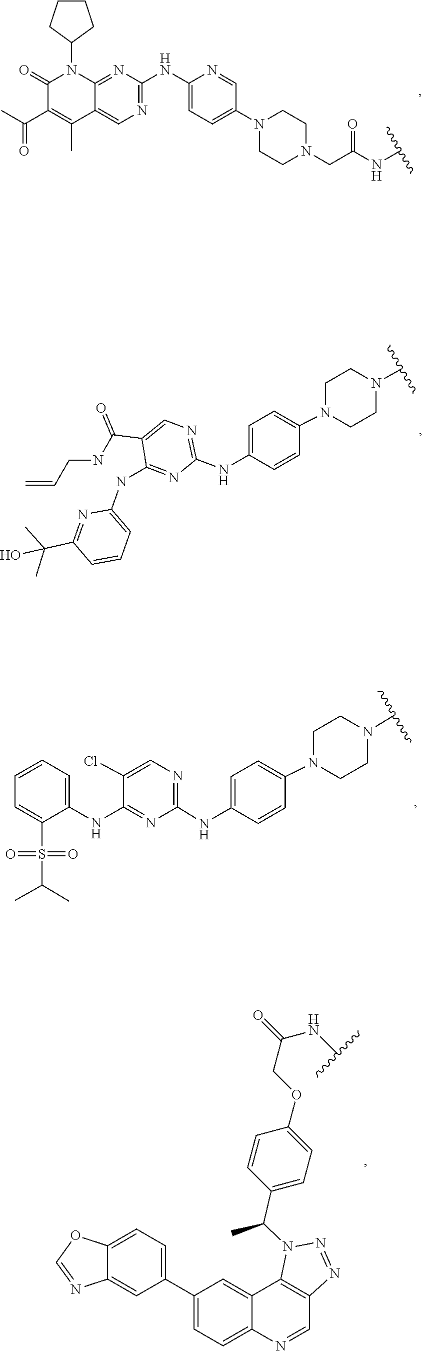

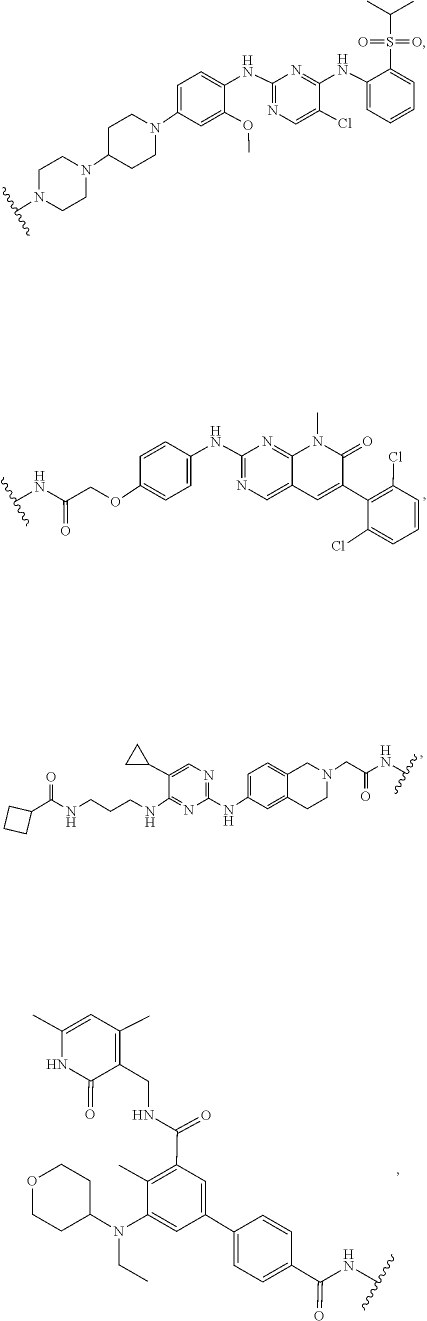 [SMILES: *N1CCN(C2CCN(c3ccc(Nc4ncc(Cl)c(Nc5ccccc5S(=O)(=O)C(C)C)n4)c(OC)c3)CC2)CC1 ; *NC(=O)COc1ccc(Nc2ncc3cc(-c4c(Cl)cccc4Cl)c(=O)n(C)c3n2)cc1 ; *NC(=O)CN1CCc2cc(Nc3ncc(C4CC4)c(NCCCNC(=O)C4CCC4)n3)ccc2C1 ; *NC(=O)c1ccc(-c2cc(C(=O)NCc3c(C)cc(C)[nH]c3=O)c(C)c(N(CC)C3CCOCC3)c2)cc1]

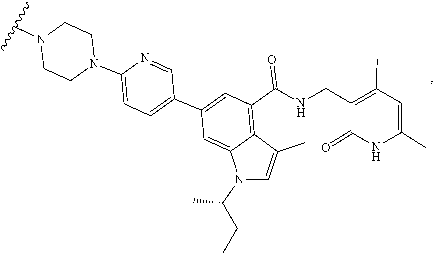 [SMILES: *N1CCN(c2ccc(-c3cc(C(=O)NCc4c(C)cc(C)[nH]c4=O)c4c(C)cn([C@@H](C)CC)c4c3)cn2)CC1]

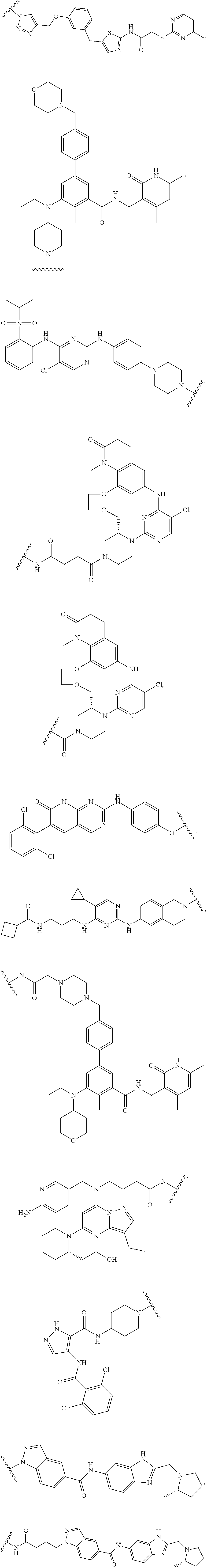

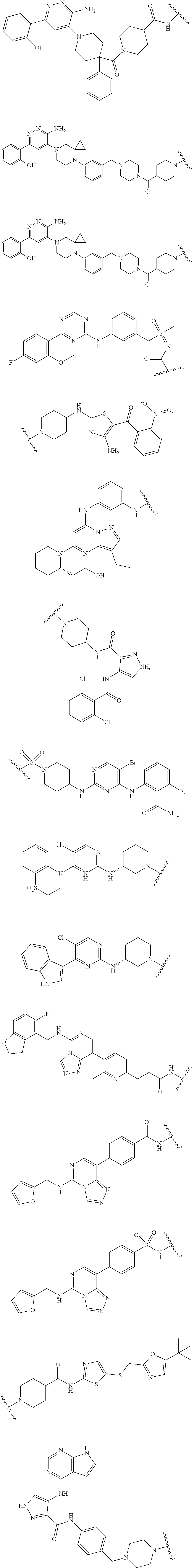

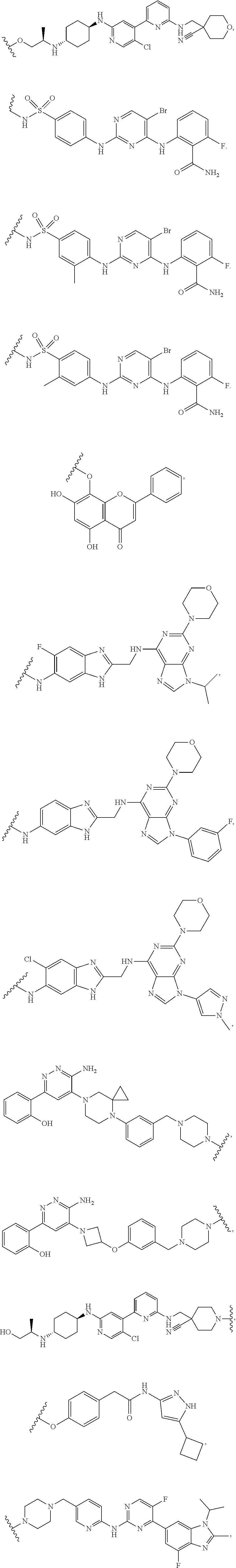

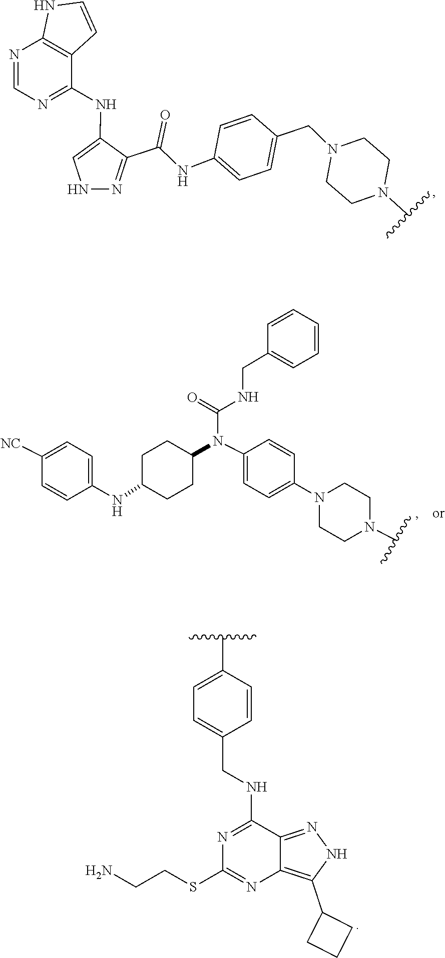 [SMILES: *N1CCN(Cc2ccc(NC(=O)c3n[nH]cc3Nc3ncnc4[nH]ccc34)cc2)CC1 ; *N1CCN(c2ccc(N(C(=O)NCc3ccccc3)[C@H]3CC[C@H](Nc4ccc(C#N)cc4)CC3)cc2)CC1 ; *c1ccc(CNc2nc(SCCN)nc3c(C4CCC4)[nH]nc23)cc1]

18. (canceled)

19. (canceled)

20. (canceled)

21. (canceled)

22. (canceled)

23. (canceled)

24. (canceled)

25. (canceled)

26. The compound of claim 10, wherein the compound has a structure of Formula IAA: 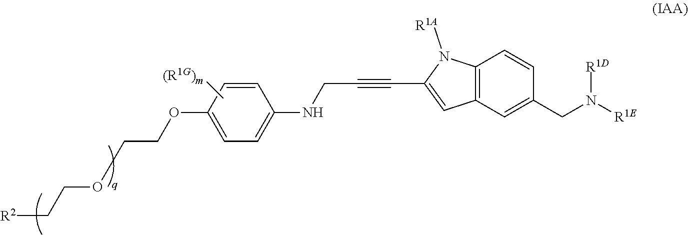
wherein:
q is an integer from 1 to 50.

27. The compound of claim 10, wherein each R^{1G} is: —CN, —F, —Cl, —OMe, —CF₃, —COOEt, —SO₂Me, —SO₂NH-pyrimidine, or two R^{1G} taken together form a 5-membered heteroaryl.

28. (canceled)

29. The compound of claim 10, wherein the compound is:

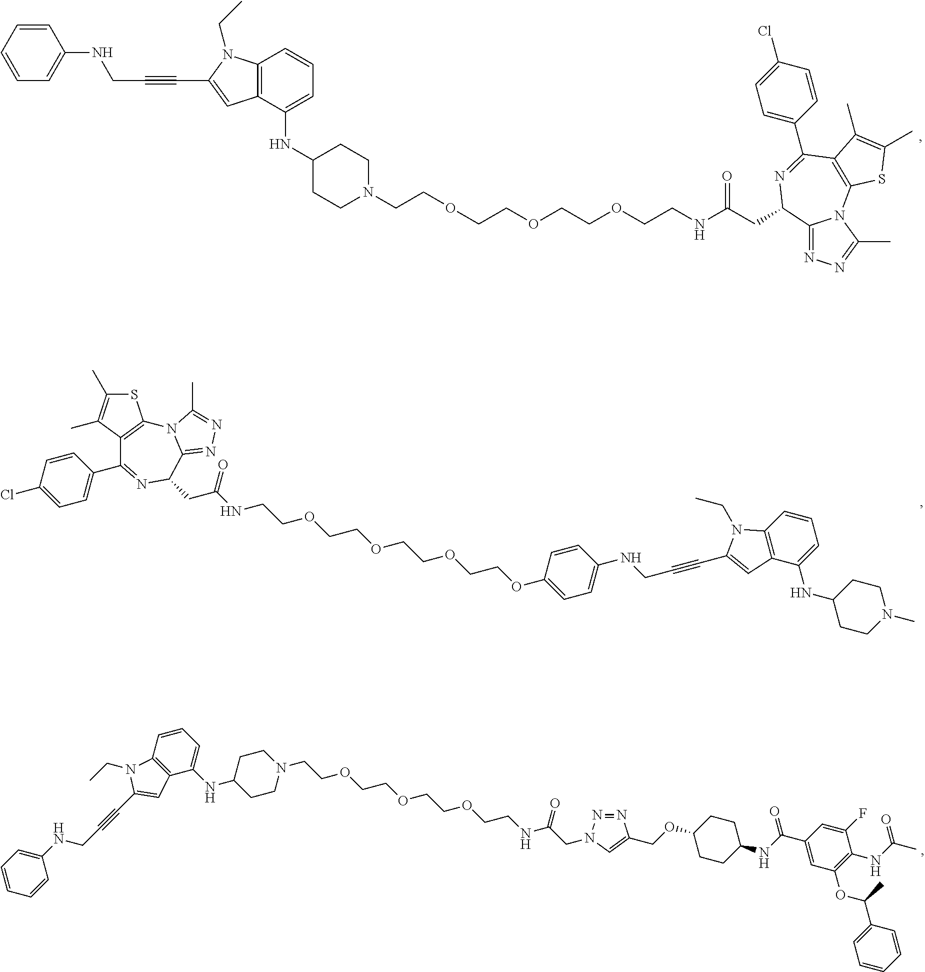 [SMILES: CCn1c(C#CCNc2ccccc2)cc2c(NC3CCN(CCOCCOCCOCCNC(=O)C[C@@H]4N=C(c5ccc(Cl)cc5)c5c(sc(C)c5C)-n5c(C)nnc54)CC3)cccc21 ; CCn1c(C#CCNc2ccc(OCCOCCOCCOCCNC(=O)C[C@@H]3N=C(c4ccc(Cl)cc4)c4c(sc(C)c4C)-n4c(C)nnc43)cc2)cc2c(NC3CCN(C)CC3)cccc21 ; CCn1c(C#CCNc2ccccc2)cc2c(NC3CCN(CCOCCOCCOCCNC(=O)Cn4cc(CO[C@H]5CC[C@H](NC(=O)c6cc(F)c(NC(C)=O)c(O[C@@H](C)c7ccccc7)c6)CC5)nn4)CC3)cccc21]

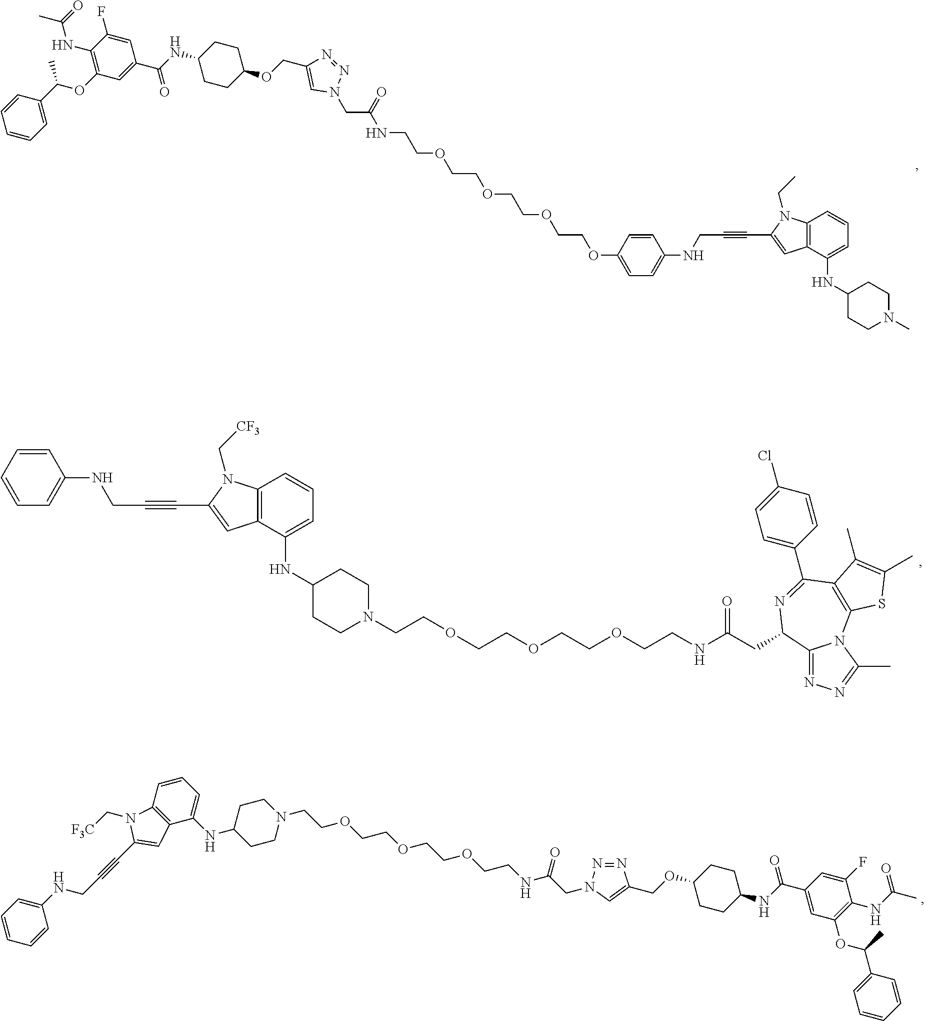 [SMILES: CCn1c(C#CCNc2ccc(OCCOCCOCCOCCNC(=O)Cn3cc(CO[C@H]4CC[C@H](NC(=O)c5cc(F)c(NC(C)=O)c(O[C@@H](C)c6ccccc6)c5)CC4)nn3)cc2)cc2c(NC3CCN(C)CC3)cccc21 ; Cc1sc2c(c1C)C(c1ccc(Cl)cc1)=N[C@@H](CC(=O)NCCOCCOCCOCCN1CCC(Nc3cccc4c3cc(C#CCNc3ccccc3)n4CC(F)(F)F)CC1)c1nnc(C)n1-2 ; CC(=O)Nc1c(F)cc(C(=O)N[C@H]2CC[C@H](OCc3cn(CC(=O)NCCOCCOCCOCCN4CCC(Nc5cccc6c5cc(C#CCNc5ccccc5)n6CC(F)(F)F)CC4)nn3)CC2)cc1O[C@@H](C)c1ccccc1]

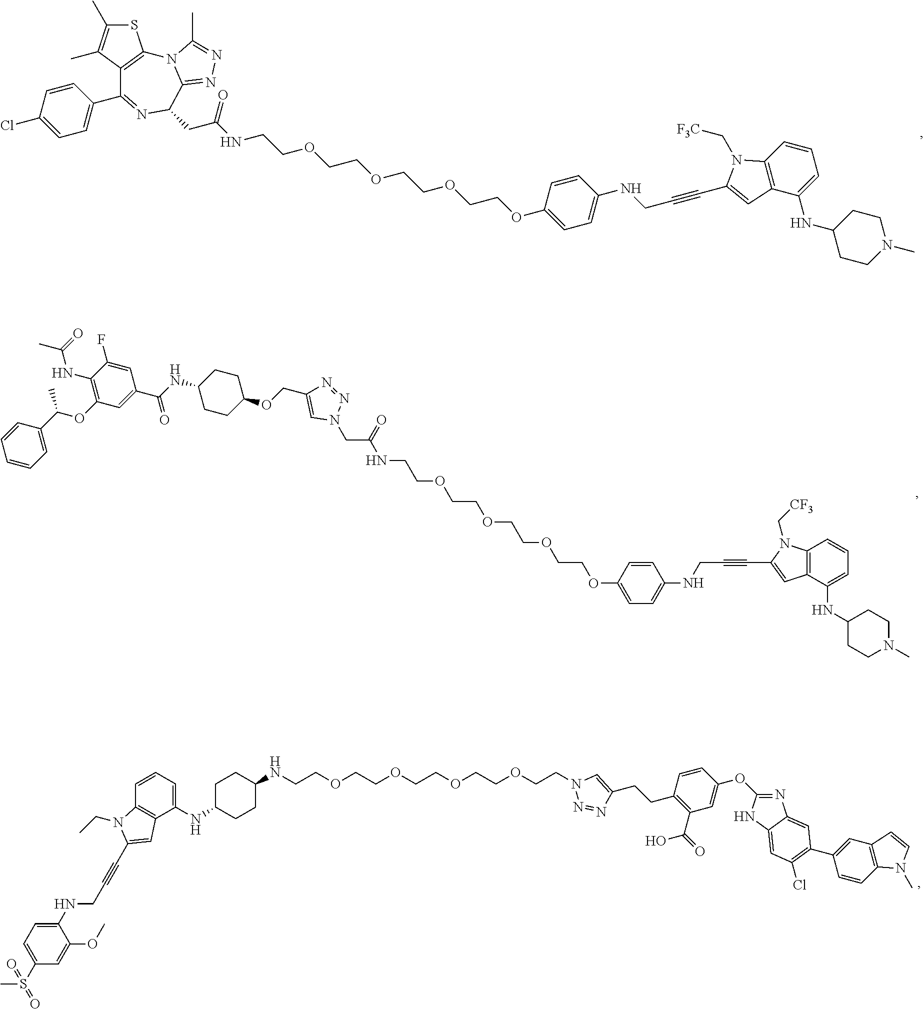

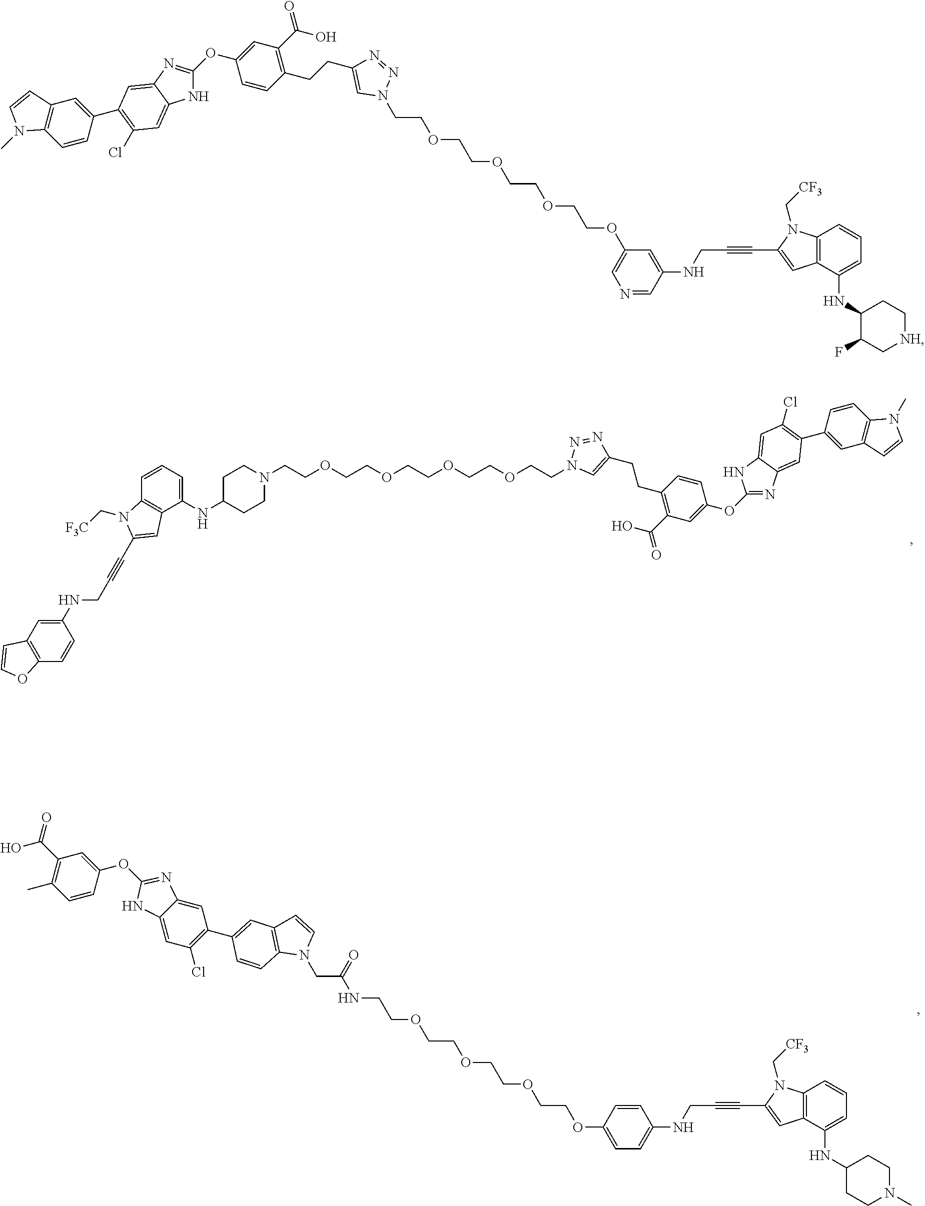 [SMILES: Cn1ccc2cc(-c3cc4nc(Oc5ccc(CCc6cn(CCOCCOCCOCCOc7cncc(NCC#Cc8cc9c(N[C@H]%10CCNC[C@H]%10F)cccc9n8CC(F)(F)F)c7)nn6)c(C(=O)O)c5)[nH]c4cc3Cl)ccc21 ; Cn1ccc2cc(-c3cc4nc(Oc5ccc(CCc6cn(CCOCCOCCOCCOCCN7CCC(Nc8cccc9c8cc(C#CCNc8ccc%10occc%10c8)n9CC(F)(F)F)CC7)nn6)c(C(=O)O)c5)[nH]c4cc3Cl)ccc21 ; Cc1ccc(Oc2nc3cc(-c4ccc5c(ccn5CC(=O)NCCOCCOCCOCCOc5ccc(NCC#Cc6cc7c(NC8CCN(C)CC8)cccc7n6CC(F)(F)F)cc5)c4)c(Cl)cc3[nH]2)cc1C(=O)O]

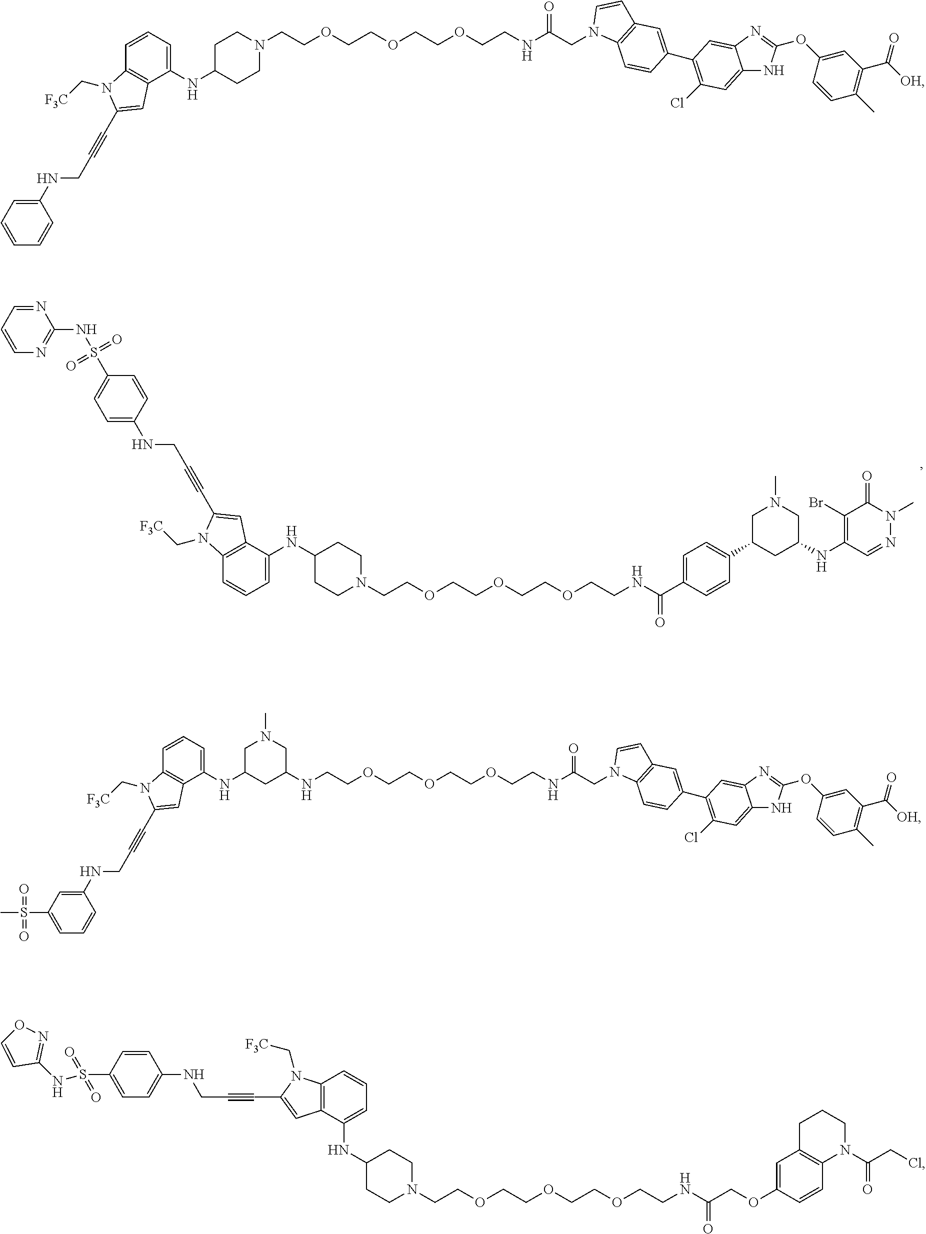

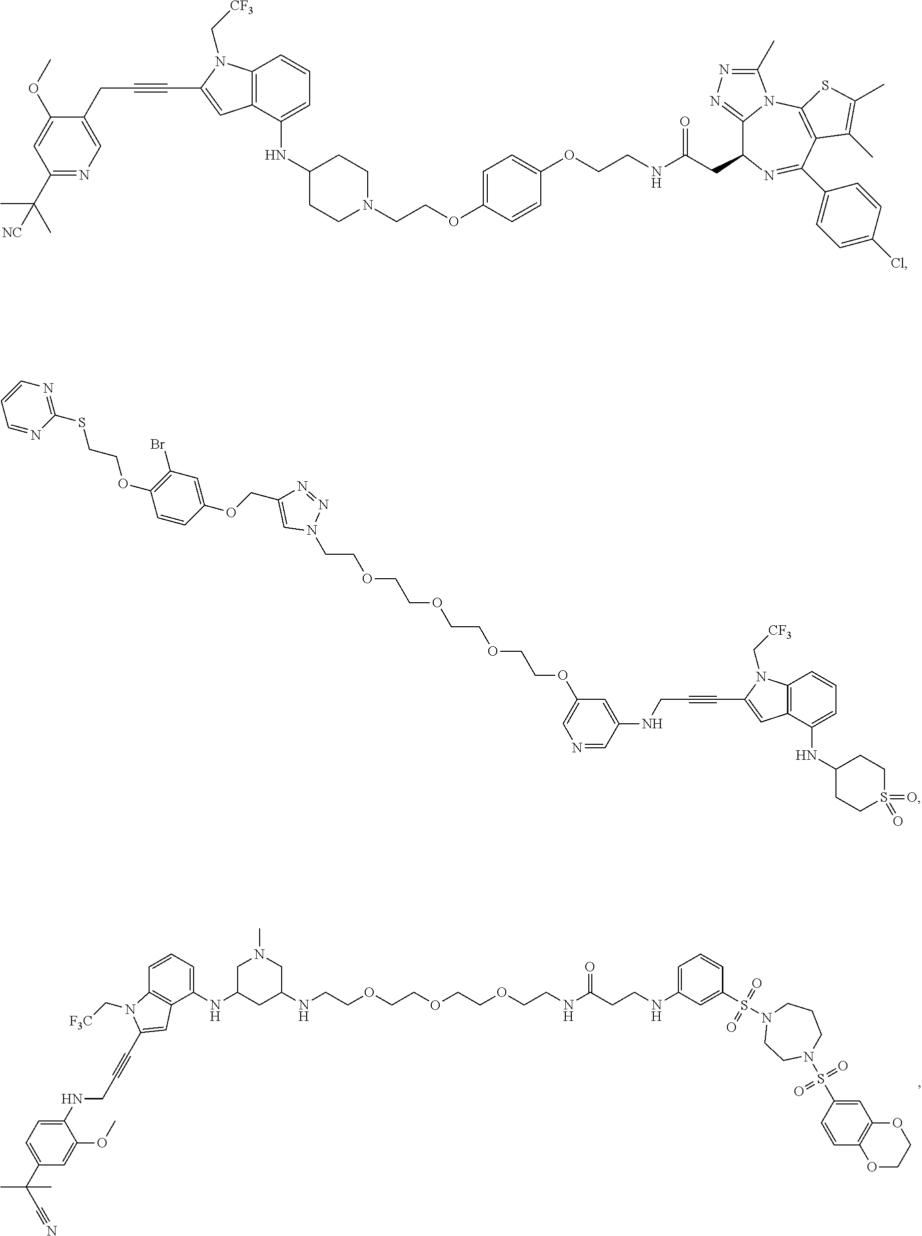 [SMILES: COc1cc(C(C)(C)C#N)ncc1CC#Cc1cc2c(NC3CCN(CCOc4ccc(OCCNC(=O)C[C@@H]5N=C(c6ccc(Cl)cc6)c6c(sc(C)c6C)-n6c(C)nnc65)cc4)CC3)cccc2n1CC(F)(F)F ; O=S1(=O)CCC(Nc2cccc3c2cc(C#CCNc2cncc(OCCOCCOCCOCCn4cc(COc5ccc(OCCSc6ncccn6)c(Br)c5)nn4)c2)n3CC(F)(F)F)CC1 ; COc1cc(C(C)(C)C#N)ccc1NCC#Cc1cc2c(NC3CC(NCCOCCOCCOCCNC(=O)CCNc4cccc(S(=O)(=O)N5CCCN(S(=O)(=O)c6ccc7c(c6)OCCO7)CC5)c4)CN(C)C3)cccc2n1CC(F)(F)F]

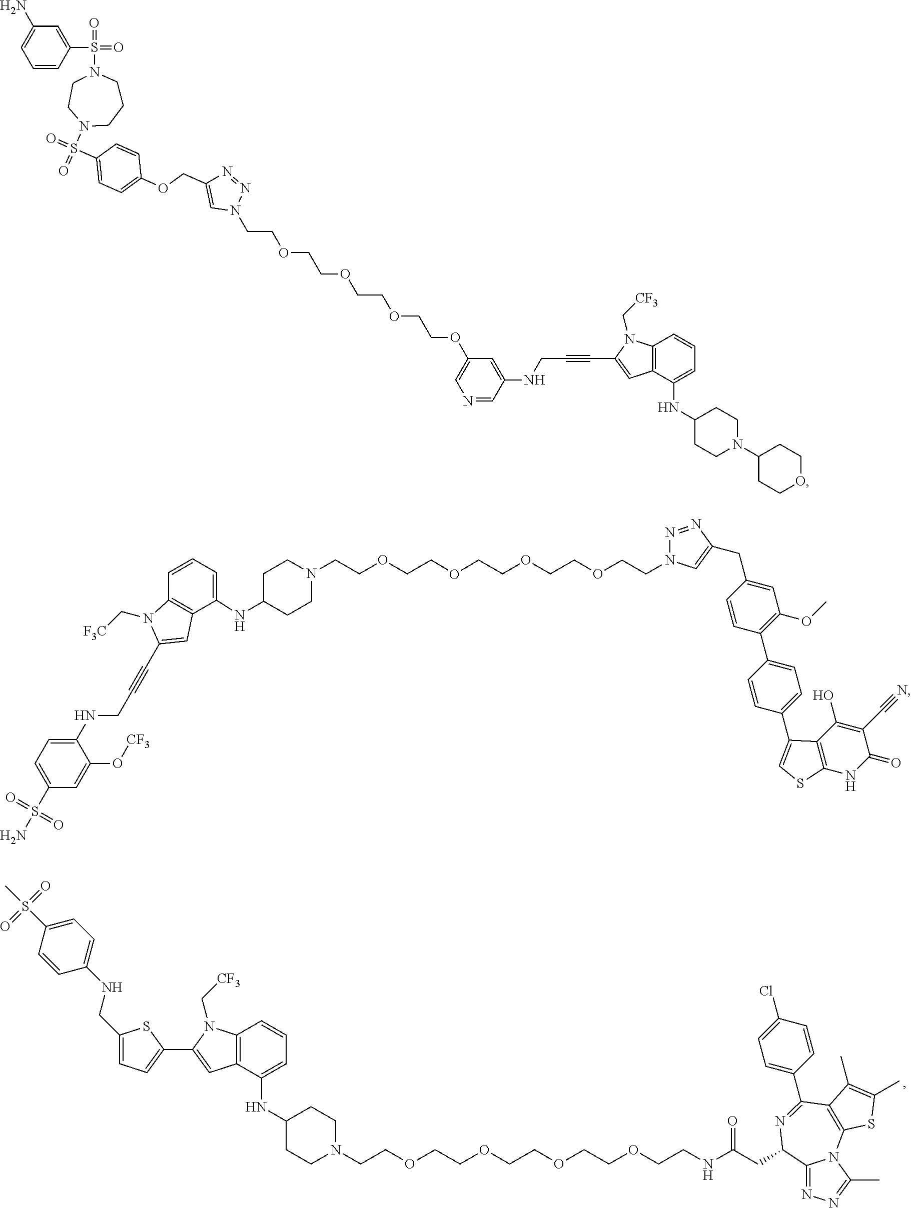

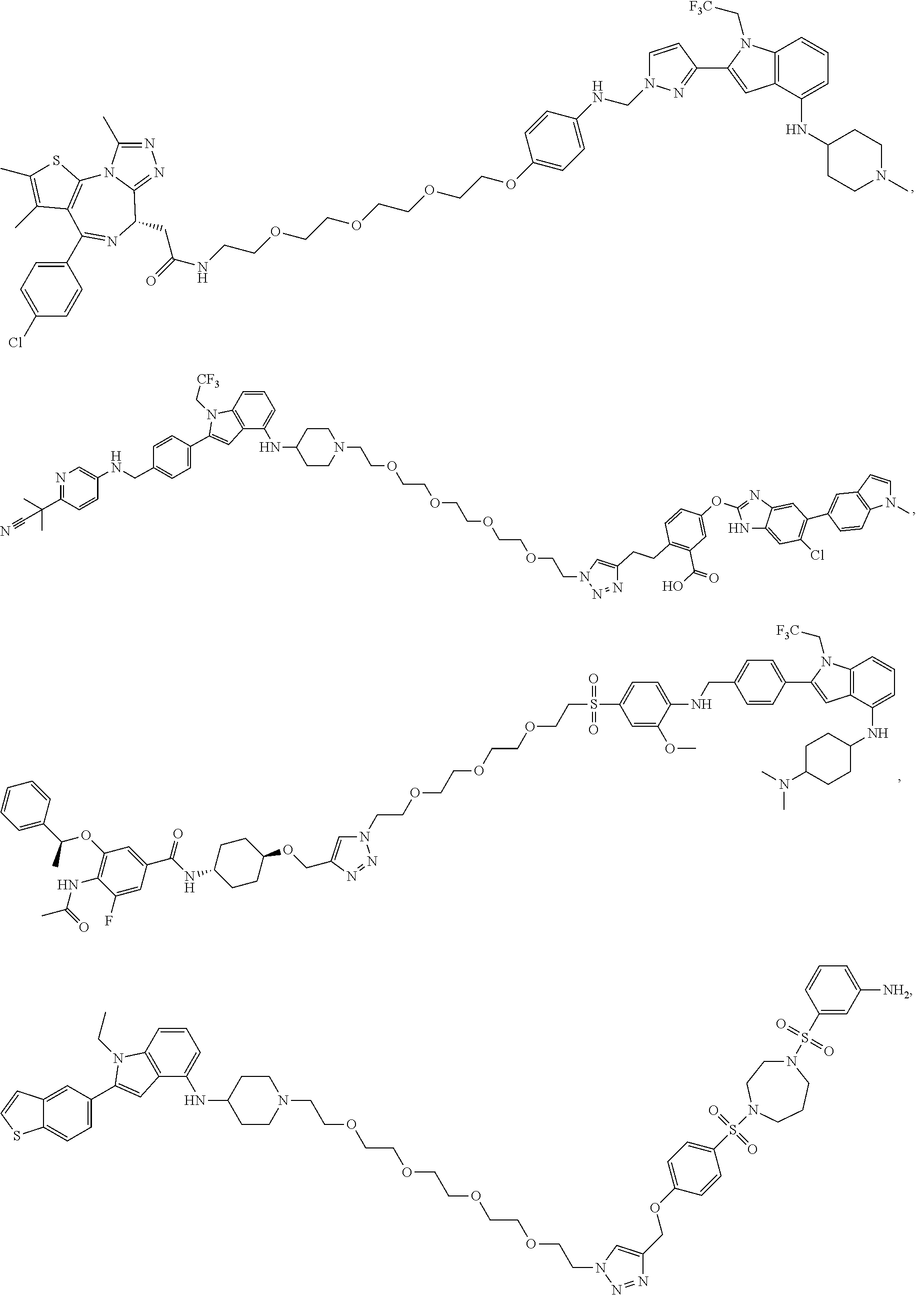

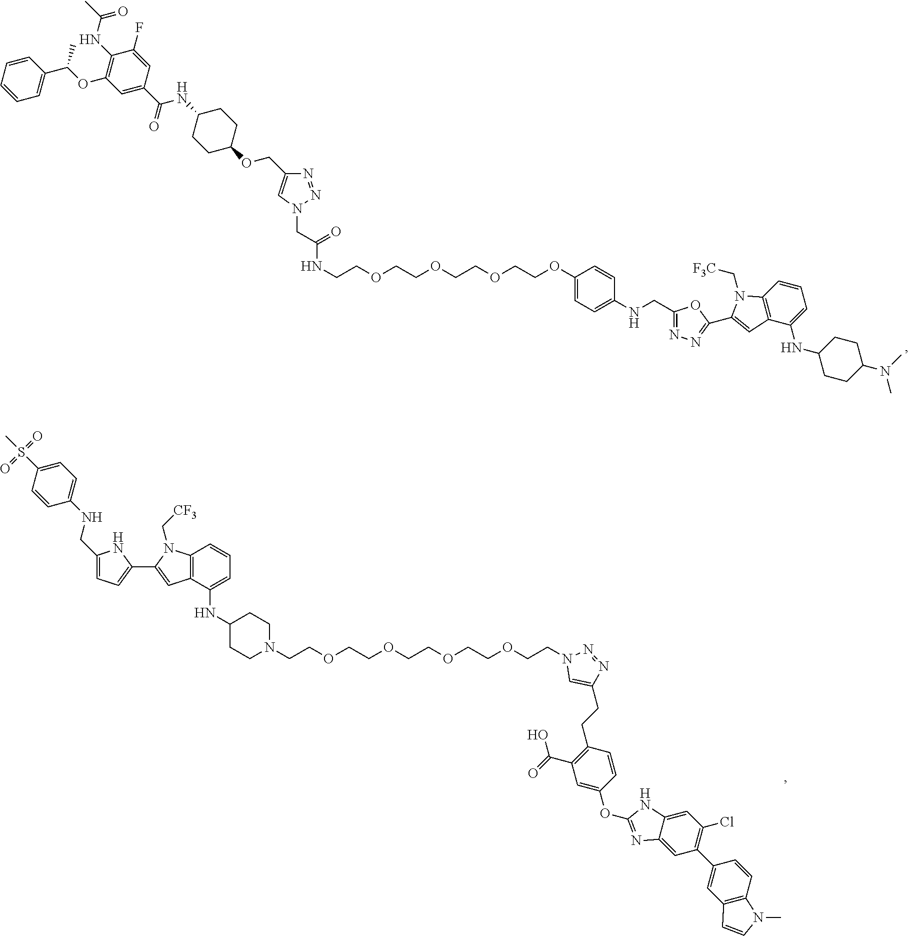 [SMILES: CC(=O)Nc1c(F)cc(C(=O)N[C@H]2CC[C@H](OCc3cn(CC(=O)NCCOCCOCCOCCOc4ccc(NCc5nnc(-c6cc7c(NC8CCC(N(C)C)CC8)cccc7n6CC(F)(F)F)o5)cc4)nn3)CC2)cc1O[C@@H](C)c1ccccc1 ; Cn1ccc2cc(-c3cc4nc(Oc5ccc(CCc6cn(CCOCCOCCOCCOCCN7CCC(Nc8cccc9c8cc(-c8ccc(CNc%10ccc(S(C)(=O)=O)cc%10)[nH]8)n9CC(F)(F)F)CC7)nn6)c(C(=O)O)c5)[nH]c4cc3Cl)ccc21]

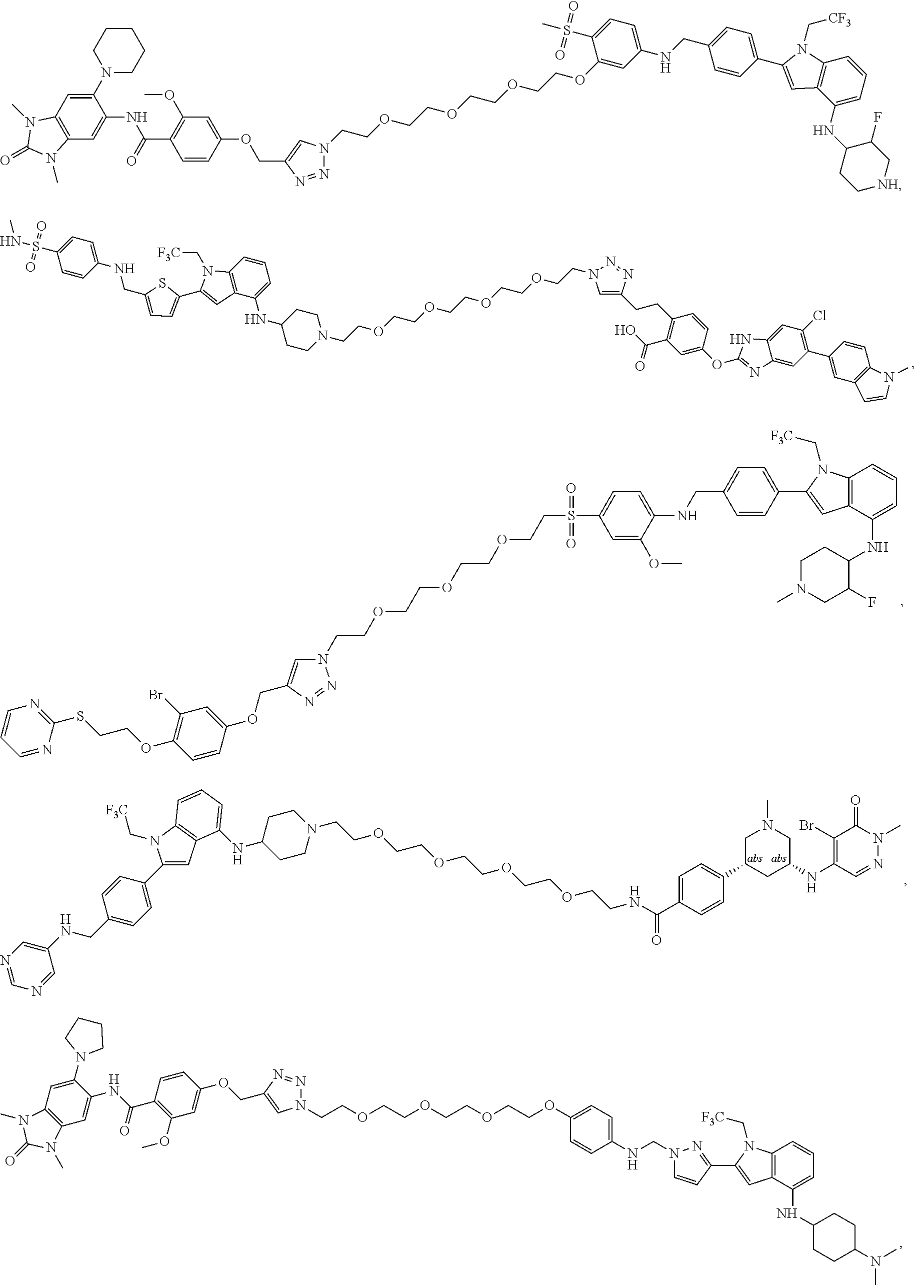

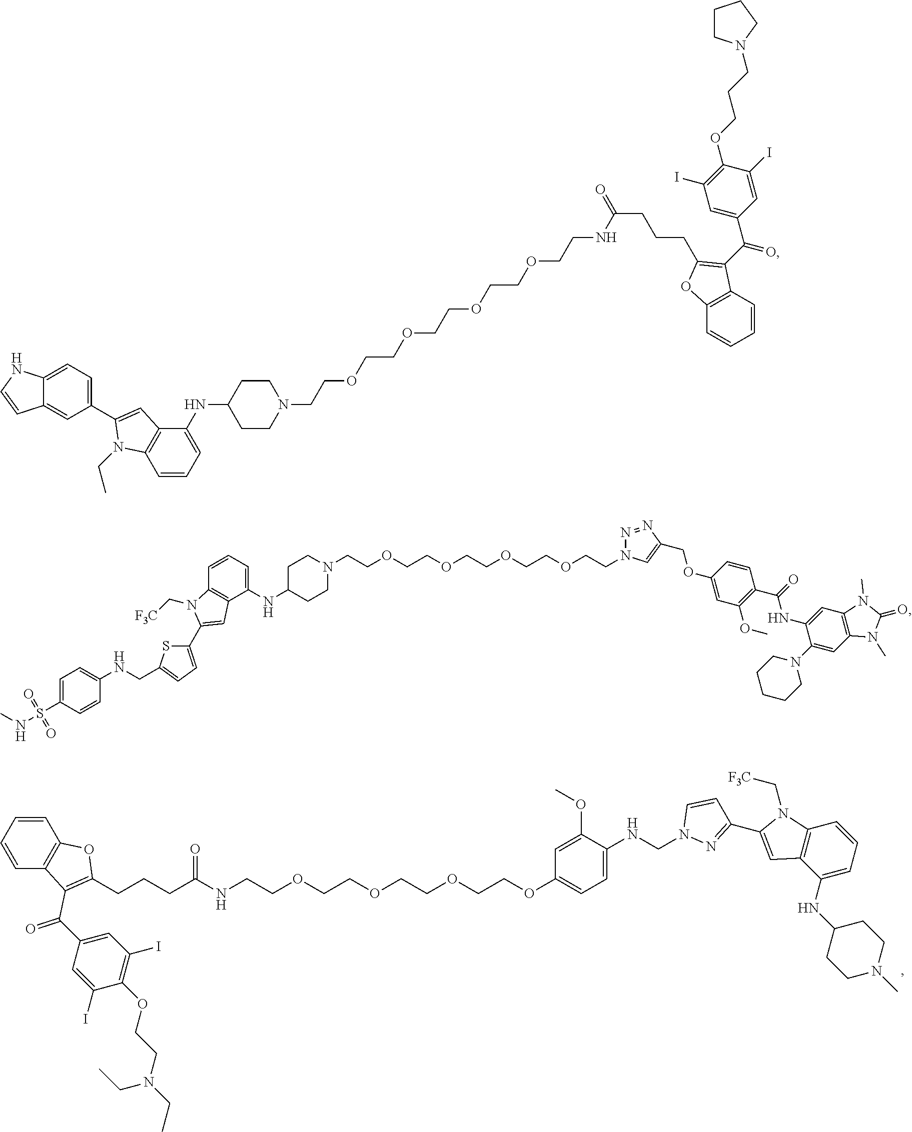 [SMILES: CCn1c(-c2ccc3[nH]ccc3c2)cc2c(NC3CCN(CCOCCOCCOCCOCCNC(=O)CCCc4oc5ccccc5c4C(=O)c4cc(I)c(OCCCN5CCCC5)c(I)c4)CC3)cccc21 ; CNS(=O)(=O)c1ccc(NCc2ccc(-c3cc4c(NC5CCN(CCOCCOCCOCCOCCn6cc(COc7ccc(C(=O)Nc8cc9c(cc8N8CCCCC8)n(C)c(=O)n9C)c(OC)c7)nn6)CC5)cccc4n3CC(F)(F)F)s2)cc1 ; CCN(CC)CCOc1c(I)cc(C(=O)c2c(CCCC(=O)NCCOCCOCCOCCOc3ccc(NCn4ccc(-c5cc6c(NC7CCN(C)CC7)cccc6n5CC(F)(F)F)n4)c(OC)c3)oc3ccccc23)cc1I]

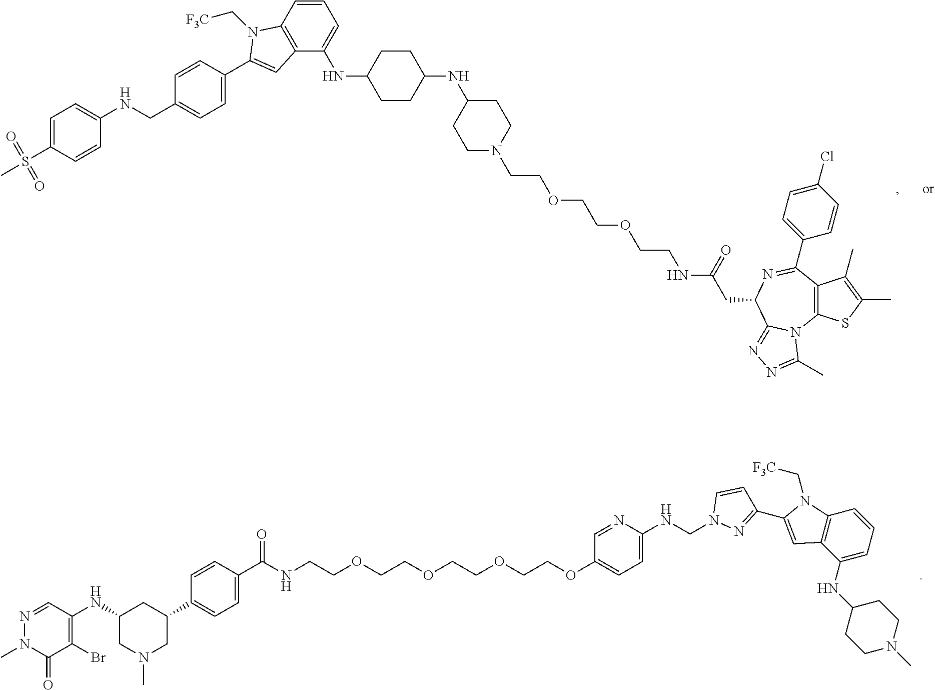

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. A method for inducing a change in a target protein comprising contacting the target protein with a modifier protein via the compound of claim 1 such that the modifier protein induces a change to the target protein.

64. (canceled)

65. (canceled)

66. (canceled)

67. (canceled)

68. A modifier protein targeting chimeric (OmniTAC) compound comprising a targeting ligand;
wherein the targeting ligand is configured to bind to a target protein; wherein the compound has a structure of: 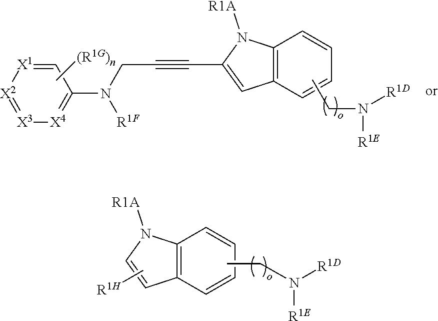
wherein:
X¹, X², and X³ are each independently N or C;
R^{1A} is a cysteine reactive group, C₁₋₆ alkyl, C₃₋₁₂ cycloalkyl, 3- to 12-membered heterocycloalkyl, —SOR^{1A1}, or —COR^{1A1}, wherein the C₁₋₆ alkyl and C₃₋₁₂ cycloalkyl is optionally substituted with —CN, halogen, or C₁₋₆alkylamines;
R^{1A1} is C₁₋₆ alkyl, C₂₋₆ alkenyl, or C₂₋₆ alkynyl, wherein the C₁₋₆ alkyl, C₂₋₆ alkenyl, and C₂₋₆ alkynyl are optionally substituted with —CN, halogen, C₁₋₆ alkyl, or C₁₋₆ alkylamines;
R^{1D} is hydrogen or C₁₋₆ alkyl;
R^{1E} is C₁₋₆ alkyl, C₃₋₁₂ cycloalkyl, 3- to 12-membered heterocycloalkyl, C₆₋₁₂ aryl, or 3- to 12-membered heteroaryl, wherein the C₁₋₆ alkyl, C₃₋₁₂ cycloalkyl, 3- to 12-membered heterocycloalkyl, C₆₋₁₂ aryl, and 3- to 12-membered heteroaryl are optionally substituted with C₁₋₆ alkyl, C₁₋₆ alkoxy, C₁₋₆ carboxylic acid, —NH₂, or —N(C₁₋₆alkyl)₂;
R^{1F} is hydrogen or C₁₋₆ alkyl;
each R^{1G} is independently —CN, halogen, C₁₋₆ alkyl, C₁₋₆ alkoxy, C₁₋₆ haloalkyl, —COO(C₁₋₆alkyl), —SO₂(C₁₋₆alkyl), —SO₂NH-heteroaryl, or two R^{1G} taken together form an aryl or heteroaryl ring;
R^{1H} is a C₆₋₁₂ aryl or 3- to 12-membered heteroaryl, wherein the C₆₋₁₂ aryl and 3- to 12-membered heteroaryl are optionally substituted with halogen, C₁₋₆ alkyl, or C₁₋₆ alkylene-NH(C₁₋₆alkyl);
n is an integer between 0 to 5; and
o is 0 or 1.

69. (canceled)

70. (canceled)

71. (canceled)

72. (canceled)

73. (canceled)

74. (canceled)

75. The compound of claim 68, wherein the compound is:

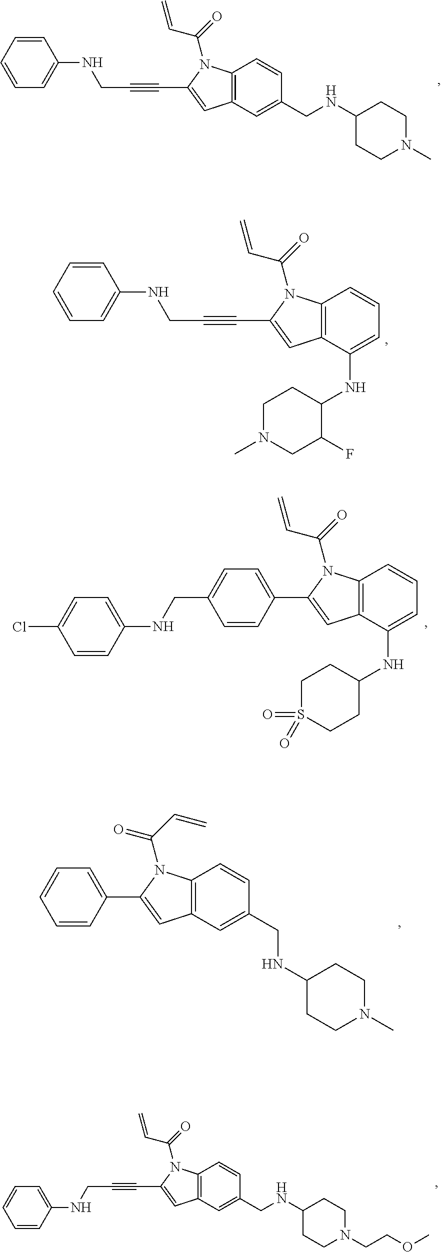

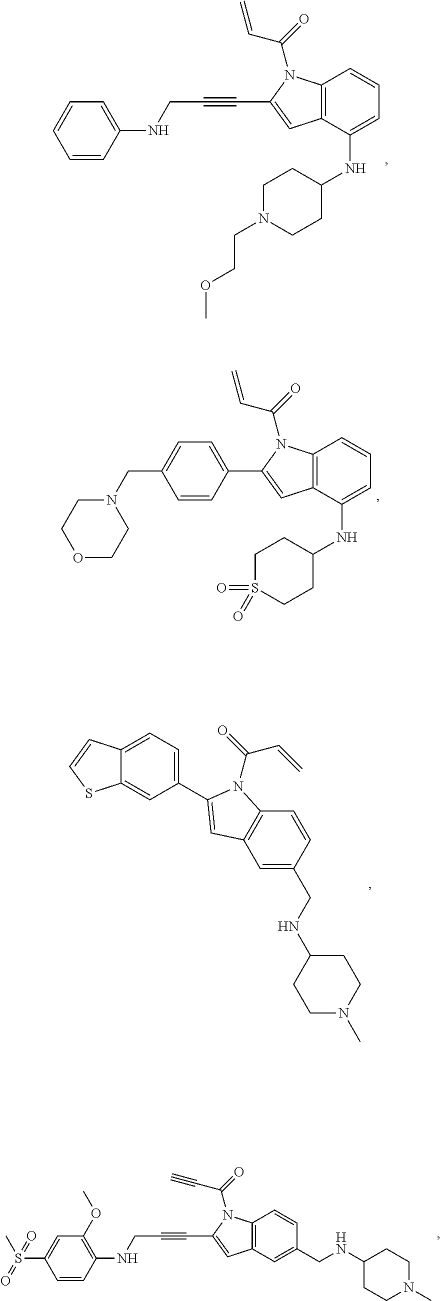

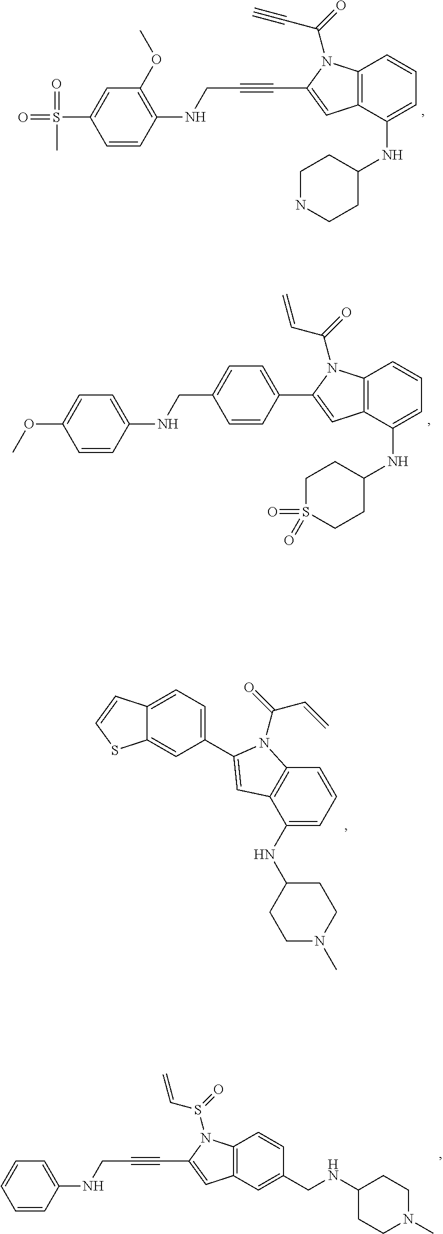 [SMILES: C#CC(=O)n1c(C#CCNc2ccc(S(C)(=O)=O)cc2OC)cc2c(NC3CCNCC3)cccc21 ; C=CC(=O)n1c(-c2ccc(CNc3ccc(OC)cc3)cc2)cc2c(NC3CCS(=O)(=O)CC3)cccc21 ; C=CC(=O)n1c(-c2ccc3ccsc3c2)cc2c(NC3CCN(C)CC3)cccc21 ; C=CS(=O)n1c(C#CCNc2ccccc2)cc2cc(CNC3CCN(C)CC3)ccc21]

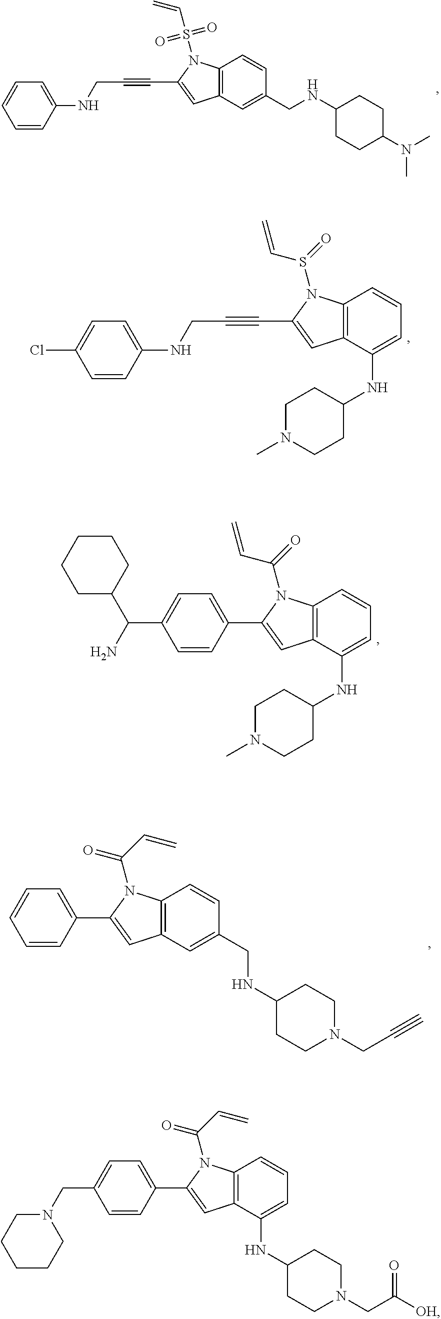 [SMILES: C=CS(=O)(=O)n1c(C#CCNc2ccccc2)cc2cc(CNC3CCC(N(C)C)CC3)ccc21 ; C=CS(=O)n1c(C#CCNc2ccc(Cl)cc2)cc2c(NC3CCN(C)CC3)cccc21 ; C=CC(=O)n1c(-c2ccc(C(N)C3CCCCC3)cc2)cc2c(NC3CCN(C)CC3)cccc21 ; C#CCN1CCC(NCc2ccc3c(c2)cc(-c2ccccc2)n3C(=O)C=C)CC1 ; C=CC(=O)n1c(-c2ccc(CN3CCCCC3)cc2)cc2c(NC3CCN(CC(=O)O)CC3)cccc21]

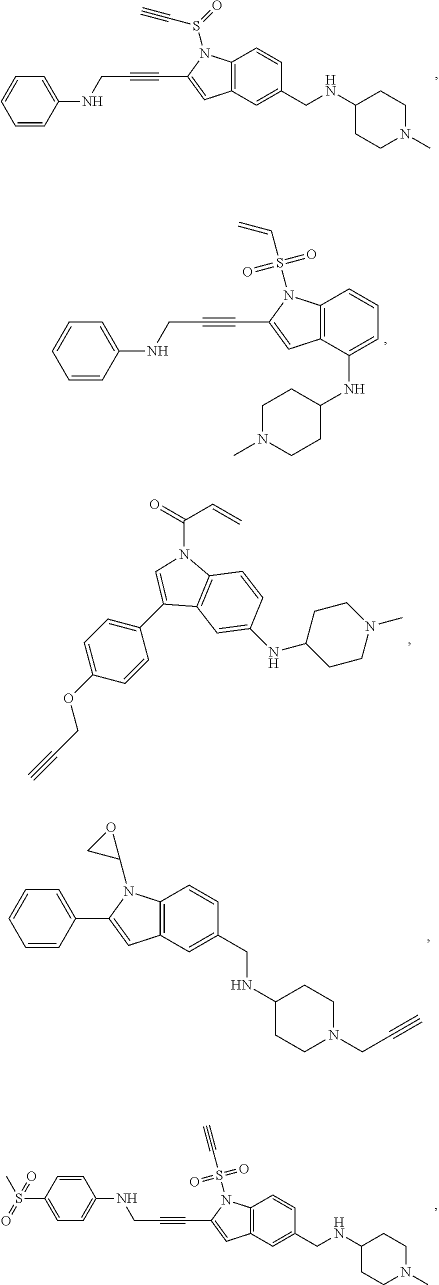

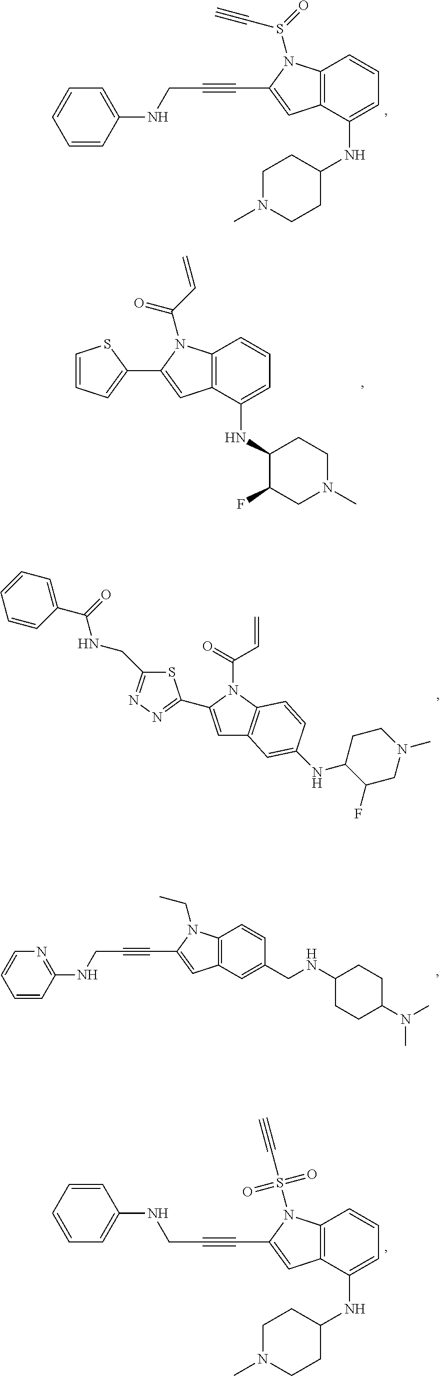 [SMILES: C#CS(=O)n1c(C#CCNc2ccccc2)cc2c(NC3CCN(C)CC3)cccc21 ; C=CC(=O)n1c(-c2cccs2)cc2c(N[C@H]3CCN(C)C[C@H]3F)cccc21 ; C=CC(=O)n1c(-c2nnc(CNC(=O)c3ccccc3)s2)cc2cc(NC3CCN(C)CC3F)ccc21 ; CCn1c(C#CCNc2ccccn2)cc2cc(CNC3CCC(N(C)C)CC3)ccc21 ; C#CS(=O)(=O)n1c(C#CCNc2ccccc2)cc2c(NC3CCN(C)CC3)cccc21]

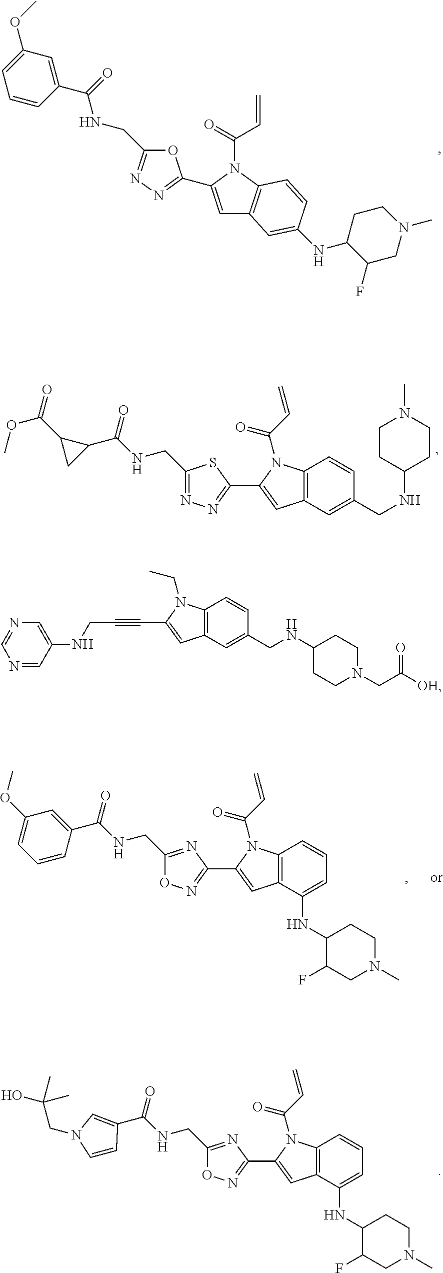

76. The compound of claim 10, wherein the compound is:

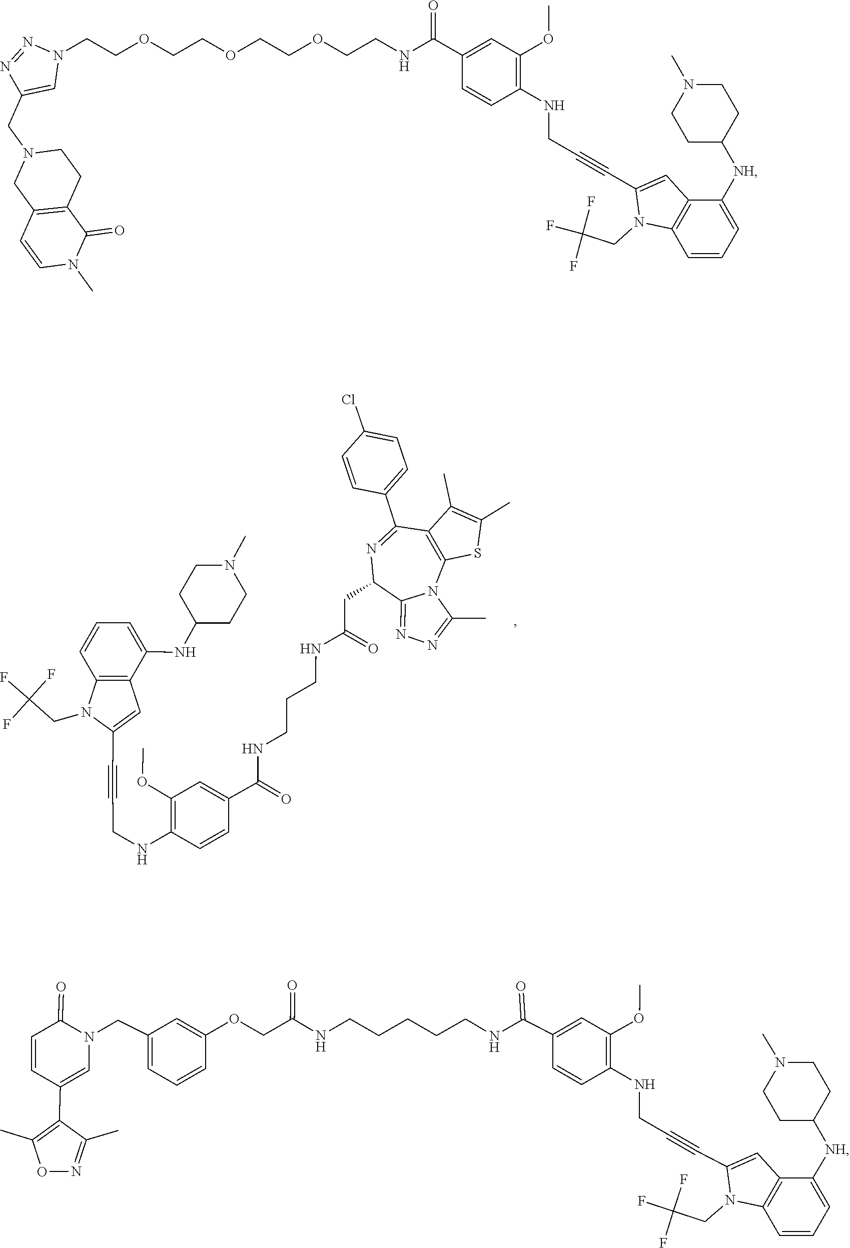

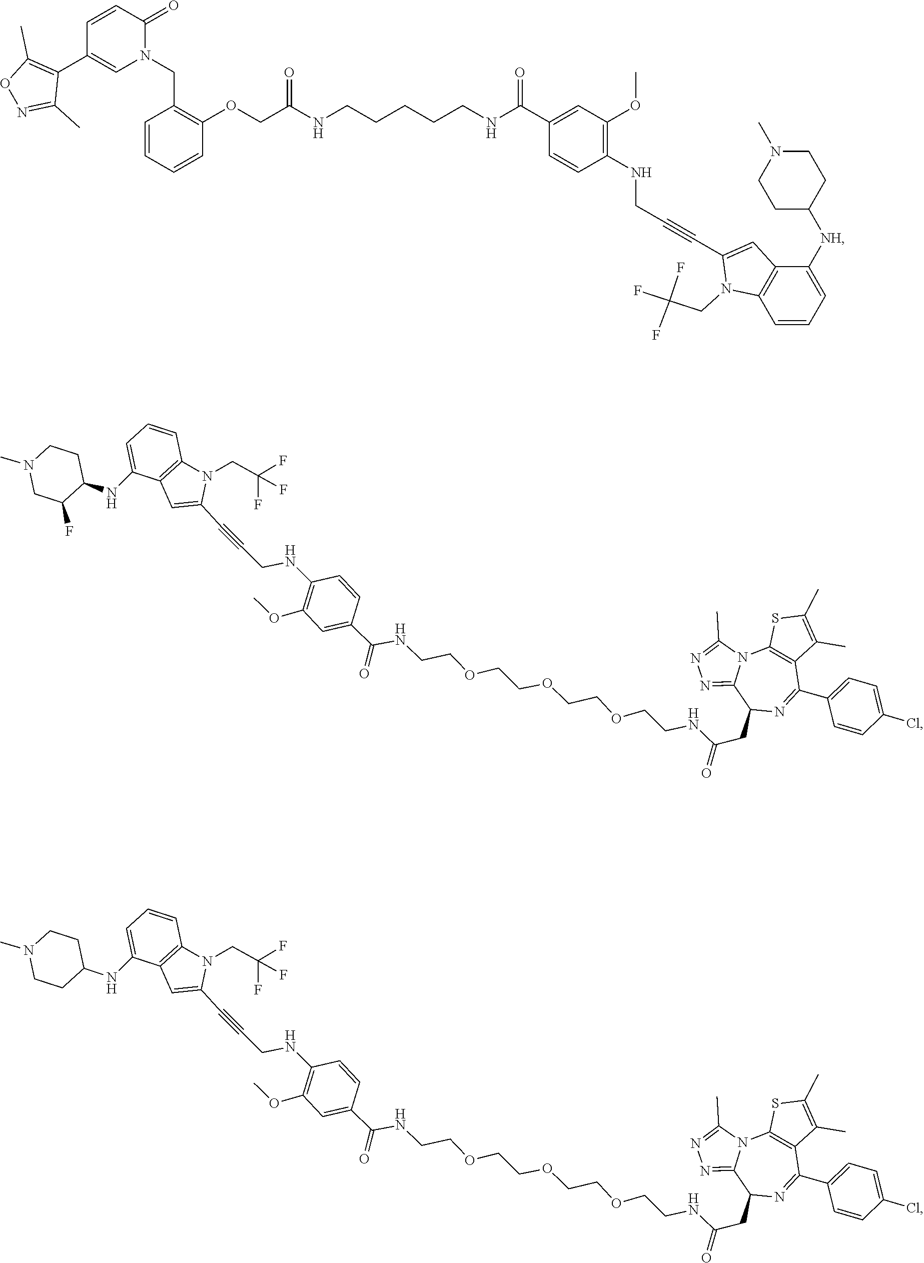 [SMILES: COc1cc(C(=O)NCCCCCNC(=O)COc2ccccc2Cn2cc(-c3c(C)noc3C)ccc2=O)ccc1NCC#Cc1cc2c(NC3CCN(C)CC3)cccc2n1CC(F)(F)F ; COc1cc(C(=O)NCCOCCOCCOCCNC(=O)C[C@@H]2N=C(c3ccc(Cl)cc3)c3c(sc(C)c3C)-n3c(C)nnc32)ccc1NCC#Cc1cc2c(N[C@@H]3CCN(C)C[C@@H]3F)cccc2n1CC(F)(F)F ; COc1cc(C(=O)NCCOCCOCCOCCNC(=O)C[C@@H]2N=C(c3ccc(Cl)cc3)c3c(sc(C)c3C)-n3c(C)nnc32)ccc1NCC#Cc1cc2c(NC3CCN(C)CC3)cccc2n1CC(F)(F)F]

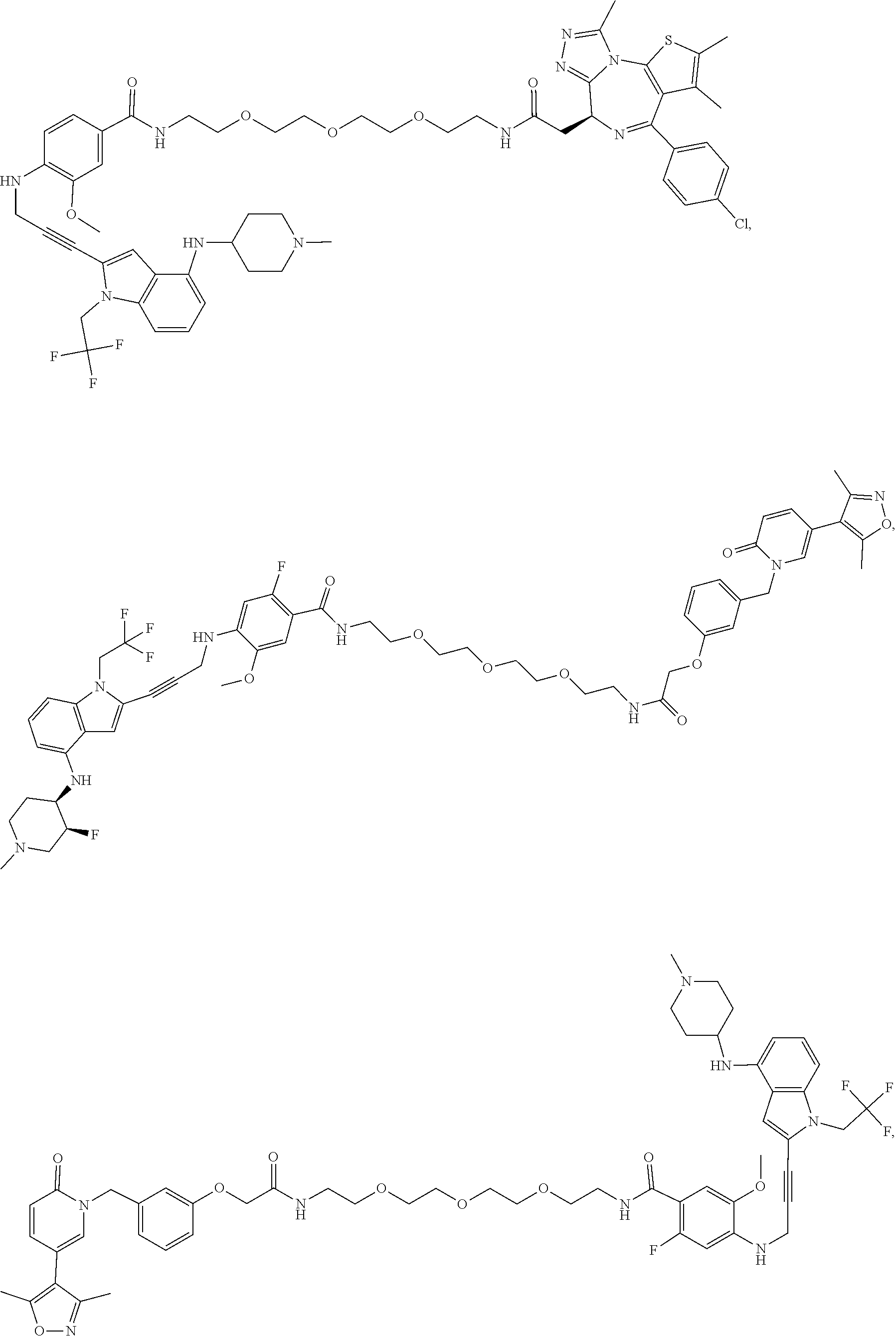 [SMILES: COc1cc(C(=O)NCCOCCOCCOCCNC(=O)C[C@@H]2N=C(c3ccc(Cl)cc3)c3c(sc(C)c3C)-n3c(C)nnc32)ccc1NCC#Cc1cc2c(NC3CCN(C)CC3)cccc2n1CC(F)(F)F ; COc1cc(C(=O)NCCOCCOCCOCCNC(=O)COc2cccc(Cn3cc(-c4c(C)noc4C)ccc3=O)c2)c(F)cc1NCC#Cc1cc2c(N[C@@H]3CCN(C)C[C@@H]3F)cccc2n1CC(F)(F)F ; COc1cc(C(=O)NCCOCCOCCOCCNC(=O)COc2cccc(Cn3cc(-c4c(C)noc4C)ccc3=O)c2)c(F)cc1NCC#Cc1cc2c(NC3CCN(C)CC3)cccc2n1CC(F)(F)F]

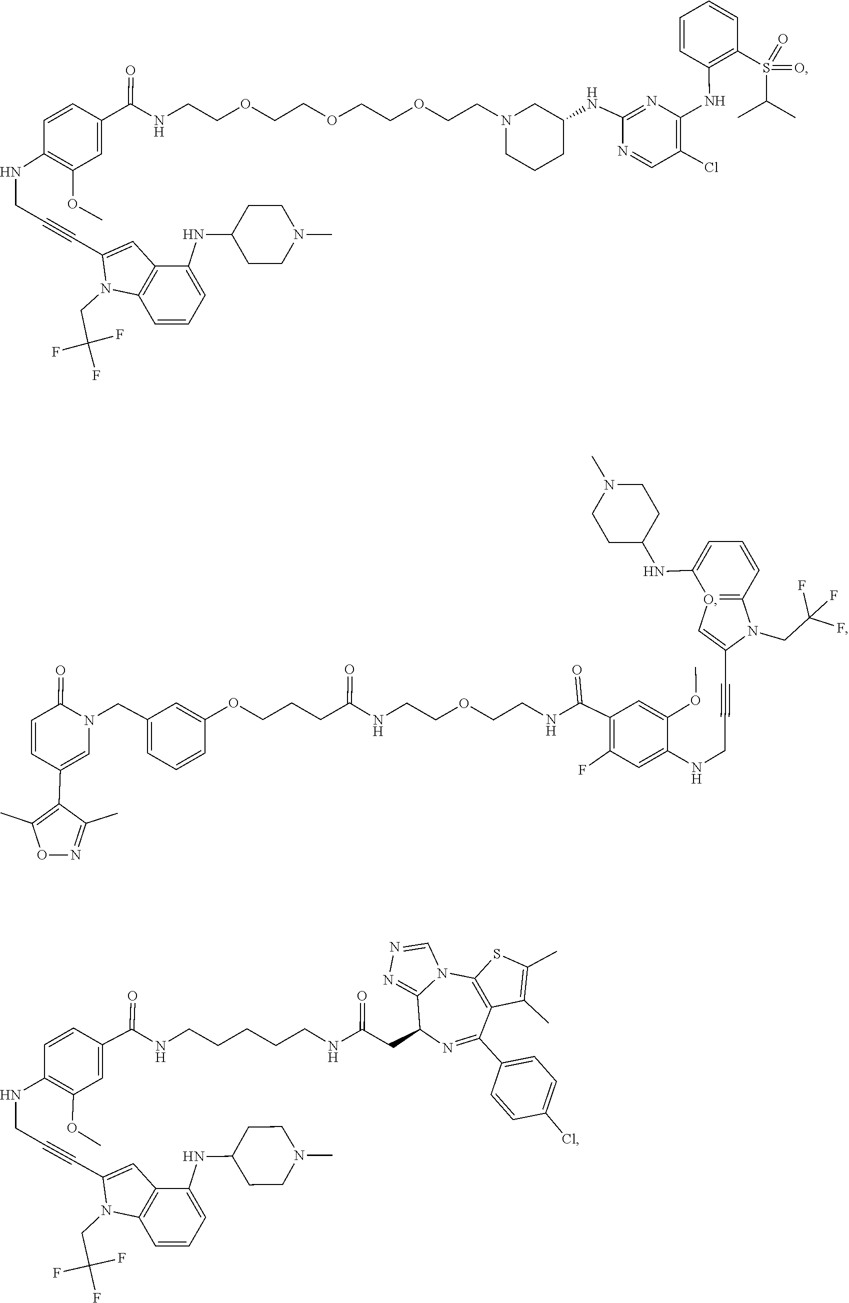

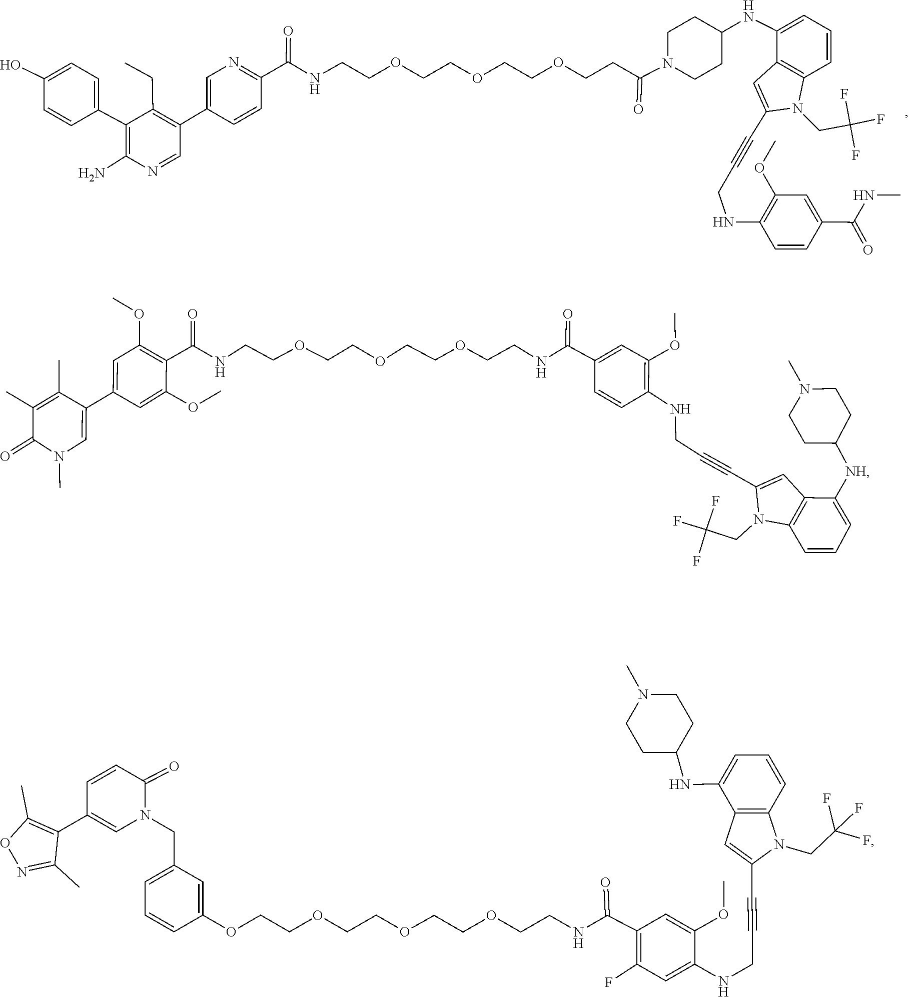 [SMILES: CCc1c(-c2ccc(C(=O)NCCOCCOCCOCCC(=O)N3CCC(Nc4cccc5c4cc(C#CCNc4ccc(C(=O)NC)cc4OC)n5CC(F)(F)F)CC3)nc2)cnc(N)c1-c1ccc(O)cc1 ; COc1cc(C(=O)NCCOCCOCCOCCNC(=O)c2c(OC)cc(-c3cn(C)c(=O)c(C)c3C)cc2OC)ccc1NCC#Cc1cc2c(NC3CCN(C)CC3)cccc2n1CC(F)(F)F ; COc1cc(C(=O)NCCOCCOCCOCCOc2cccc(Cn3cc(-c4c(C)noc4C)ccc3=O)c2)c(F)cc1NCC#Cc1cc2c(NC3CCN(C)CC3)cccc2n1CC(F)(F)F]

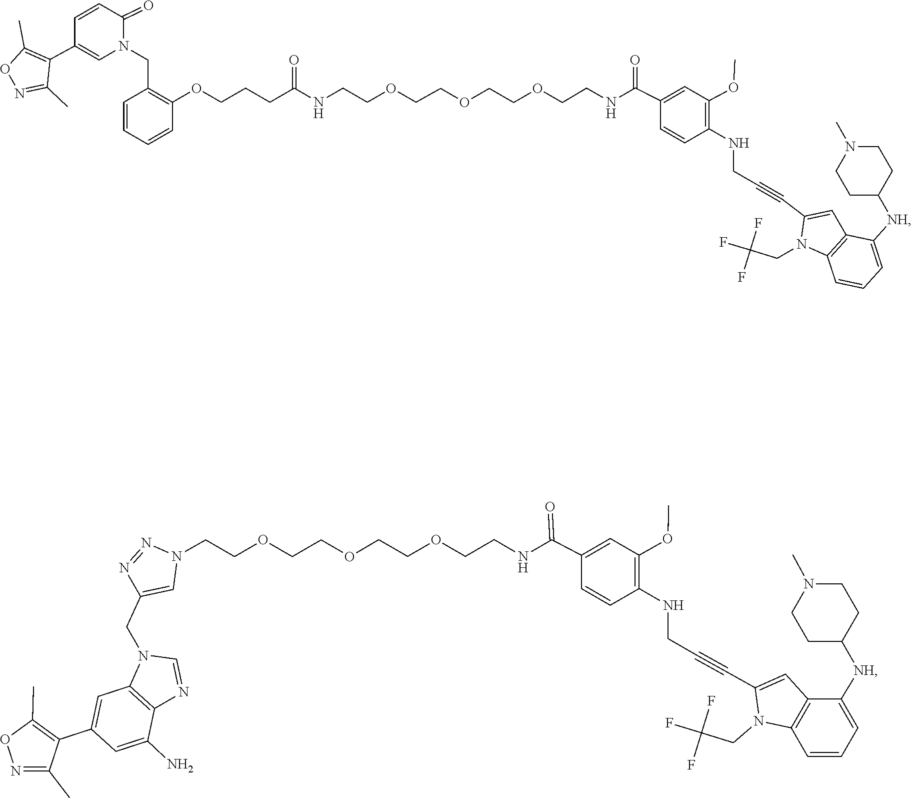 [SMILES: COc1cc(C(=O)NCCOCCOCCOCCNC(=O)CCCOc2ccccc2Cn2cc(-c3c(C)noc3C)ccc2=O)ccc1NCC#Cc1cc2c(NC3CCN(C)CC3)cccc2n1CC(F)(F)F ; COc1cc(C(=O)NCCOCCOCCOCCn2cc(Cn3cnc4c(N)cc(-c5c(C)noc5C)cc43)nn2)ccc1NCC#Cc1cc2c(NC3CCN(C)CC3)cccc2n1CC(F)(F)F]

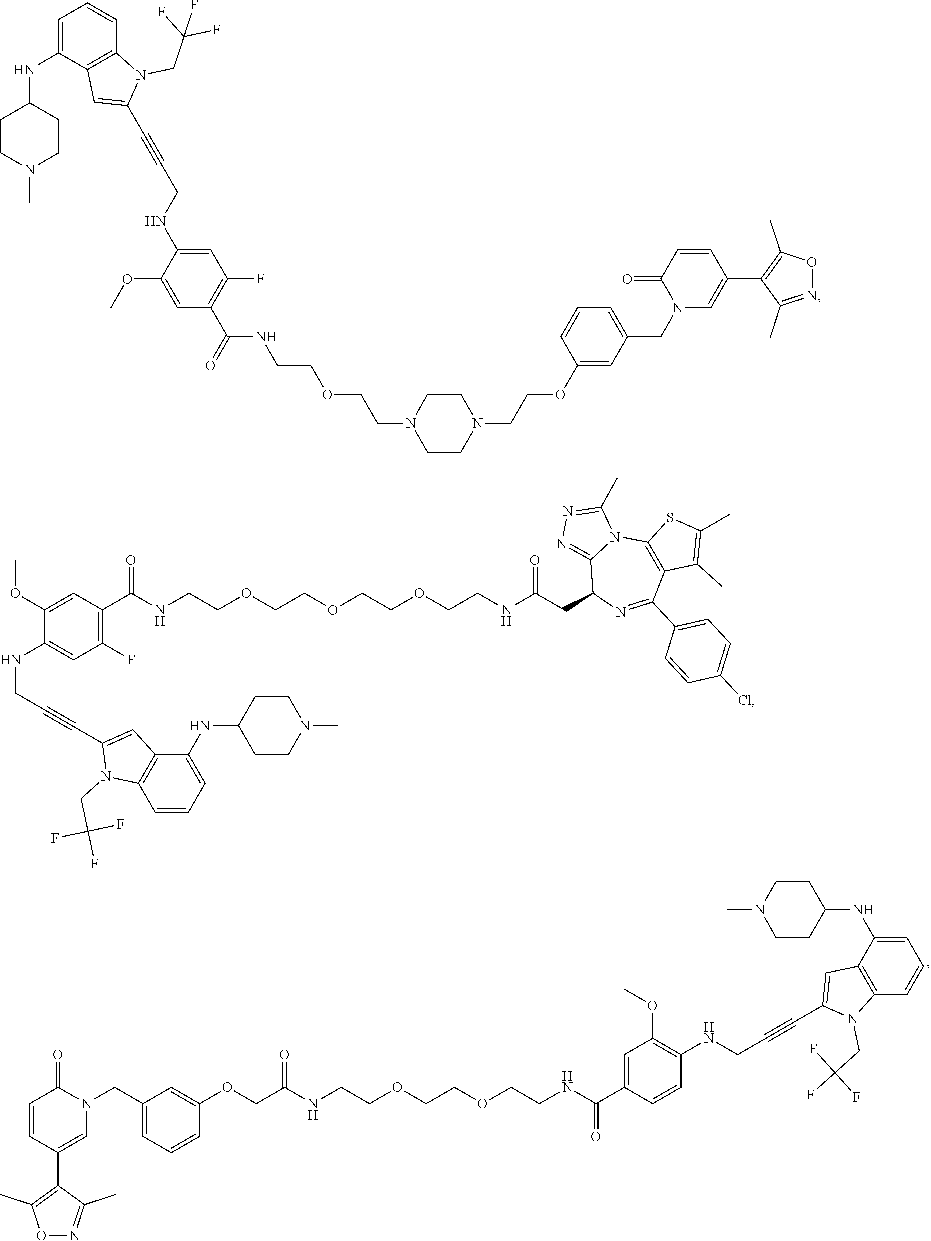 [SMILES: COc1cc(C(=O)NCCOCCN2CCN(CCOc3cccc(Cn4cc(-c5c(C)noc5C)ccc4=O)c3)CC2)c(F)cc1NCC#Cc1cc2c(NC3CCN(C)CC3)cccc2n1CC(F)(F)F ; COc1cc(C(=O)NCCOCCOCCOCCNC(=O)C[C@@H]2N=C(c3ccc(Cl)cc3)c3c(sc(C)c3C)-n3c(C)nnc32)c(F)cc1NCC#Cc1cc2c(NC3CCN(C)CC3)cccc2n1CC(F)(F)F ; COc1cc(C(=O)NCCOCCOCCNC(=O)COc2cccc(Cn3cc(-c4c(C)noc4C)ccc3=O)c2)ccc1NCC#Cc1cc2c(NC3CCN(C)CC3)cccc2n1CC(F)(F)F]

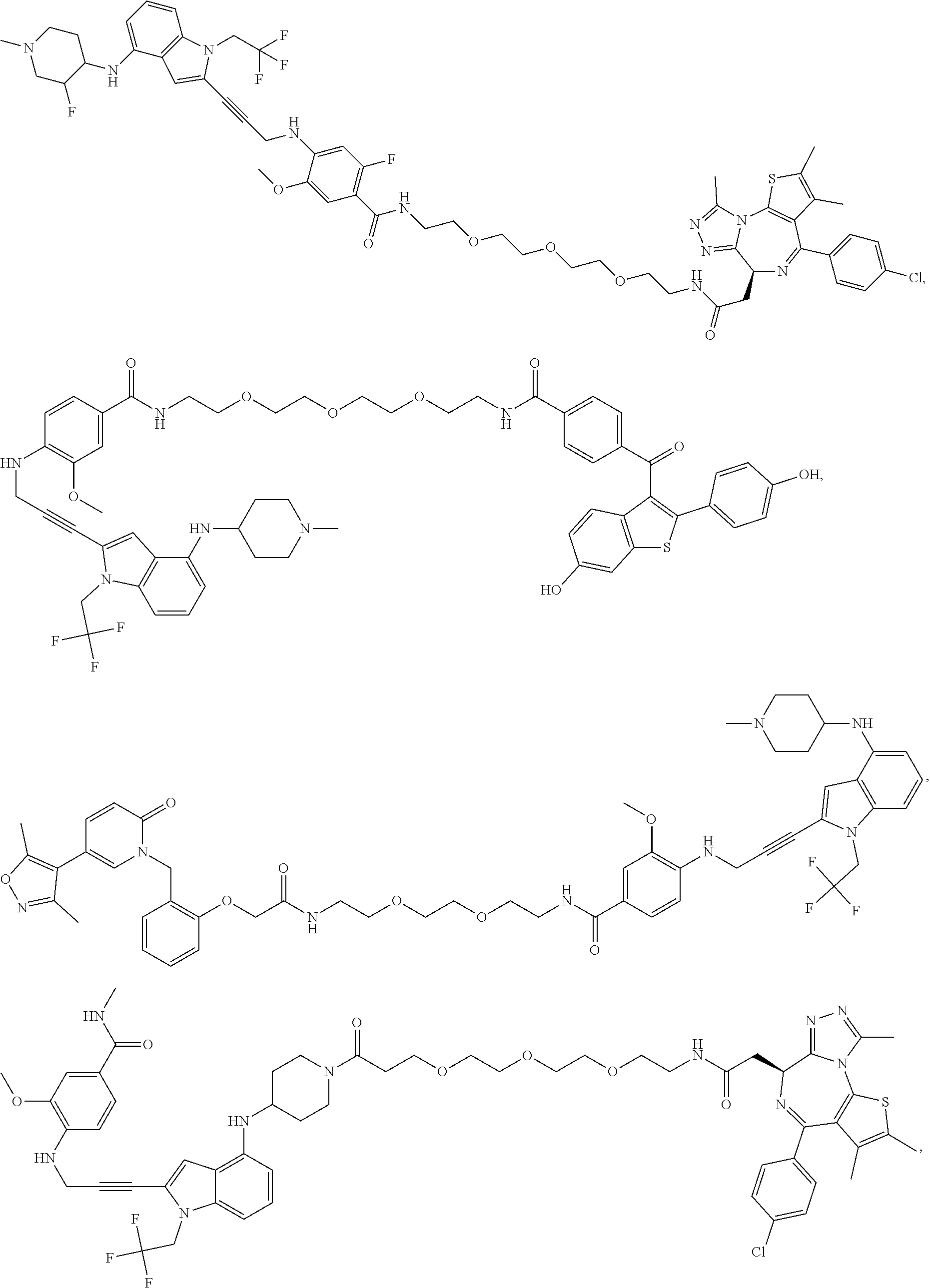

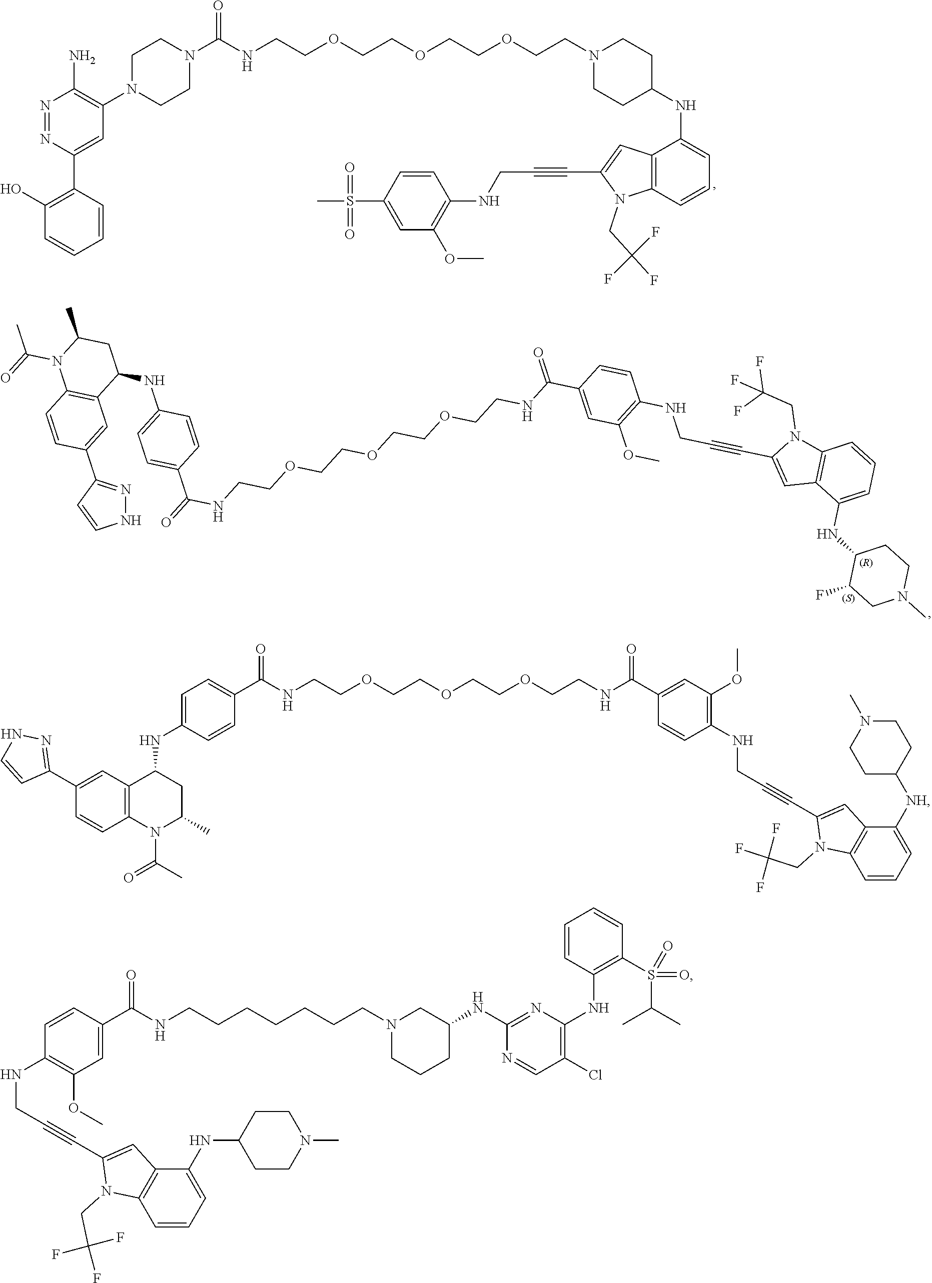

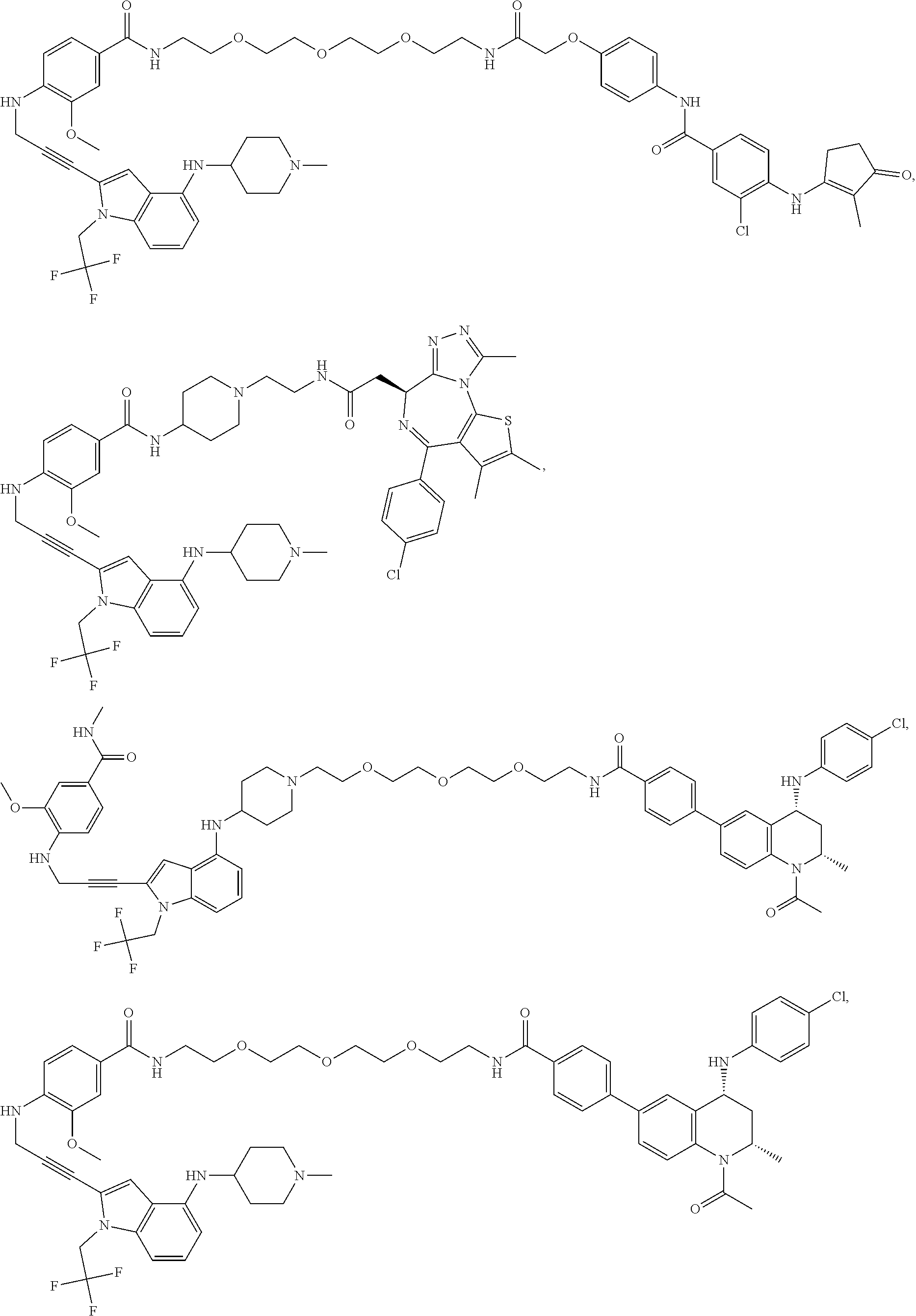

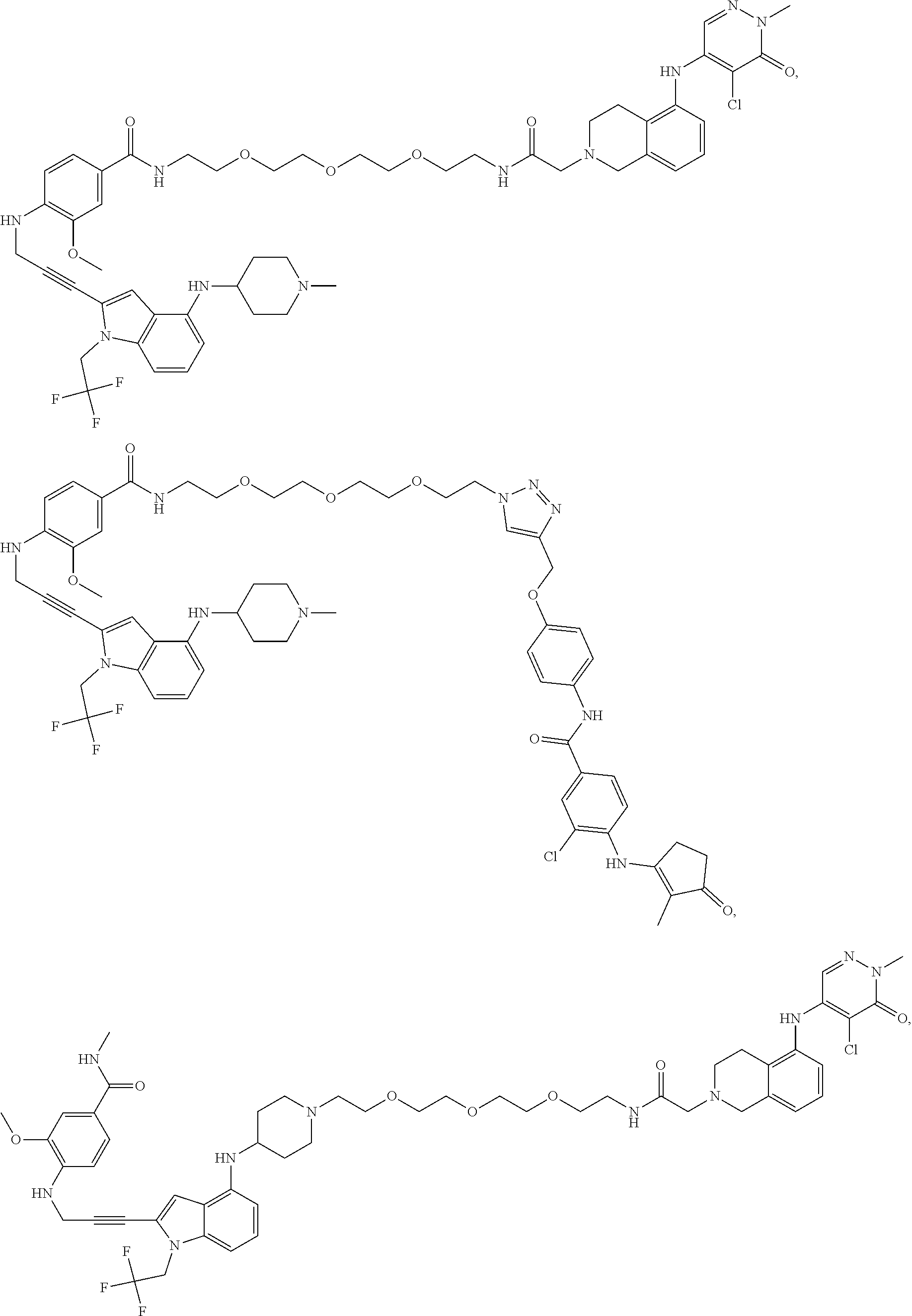

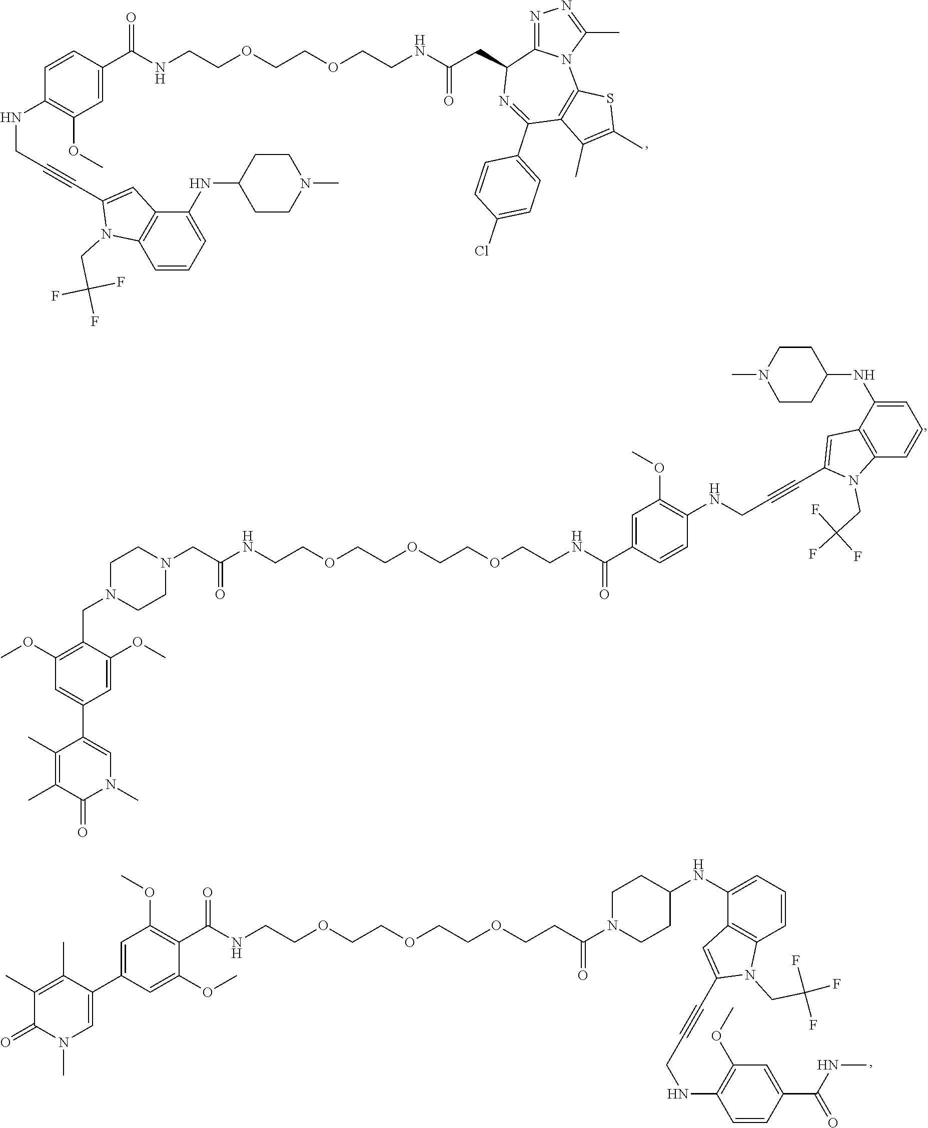

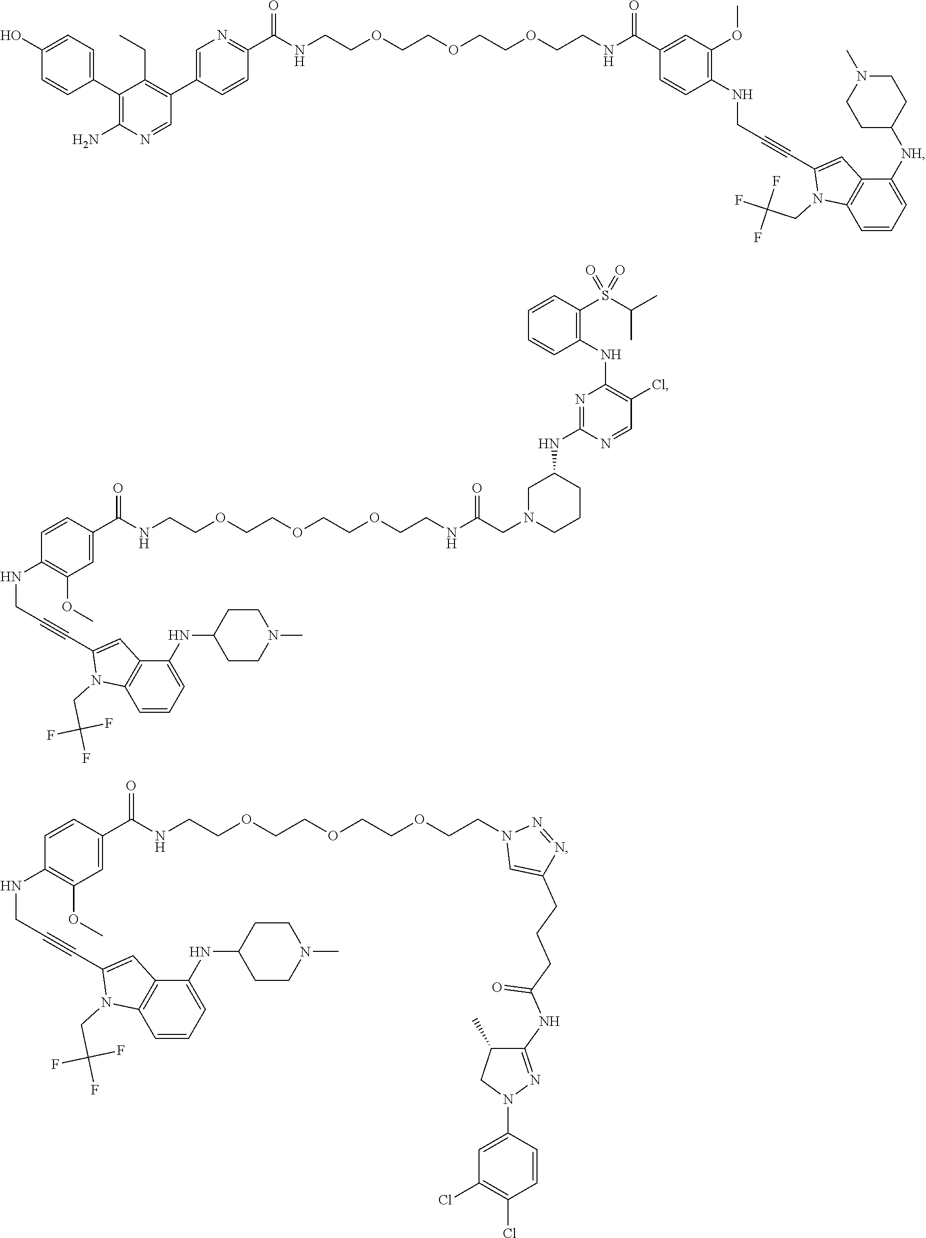 [SMILES: CCc1c(-c2ccc(C(=O)NCCOCCOCCOCCNC(=O)c3ccc(NCC#Cc4cc5c(NC6CCN(C)CC6)cccc5n4CC(F)(F)F)c(OC)c3)nc2)cnc(N)c1-c1ccc(O)cc1 ; COc1cc(C(=O)NCCOCCOCCOCCNC(=O)CN2CCC[C@@H](Nc3ncc(Cl)c(Nc4ccccc4S(=O)(=O)C(C)C)n3)C2)ccc1NCC#Cc1cc2c(NC3CCN(C)CC3)cccc2n1CC(F)(F)F ; COc1cc(C(=O)NCCOCCOCCOCCn2cc(CCCC(=O)NC3=NN(c4ccc(Cl)c(Cl)c4)C[C@@H]3C)nn2)ccc1NCC#Cc1cc2c(NC3CCN(C)CC3)cccc2n1CC(F)(F)F]

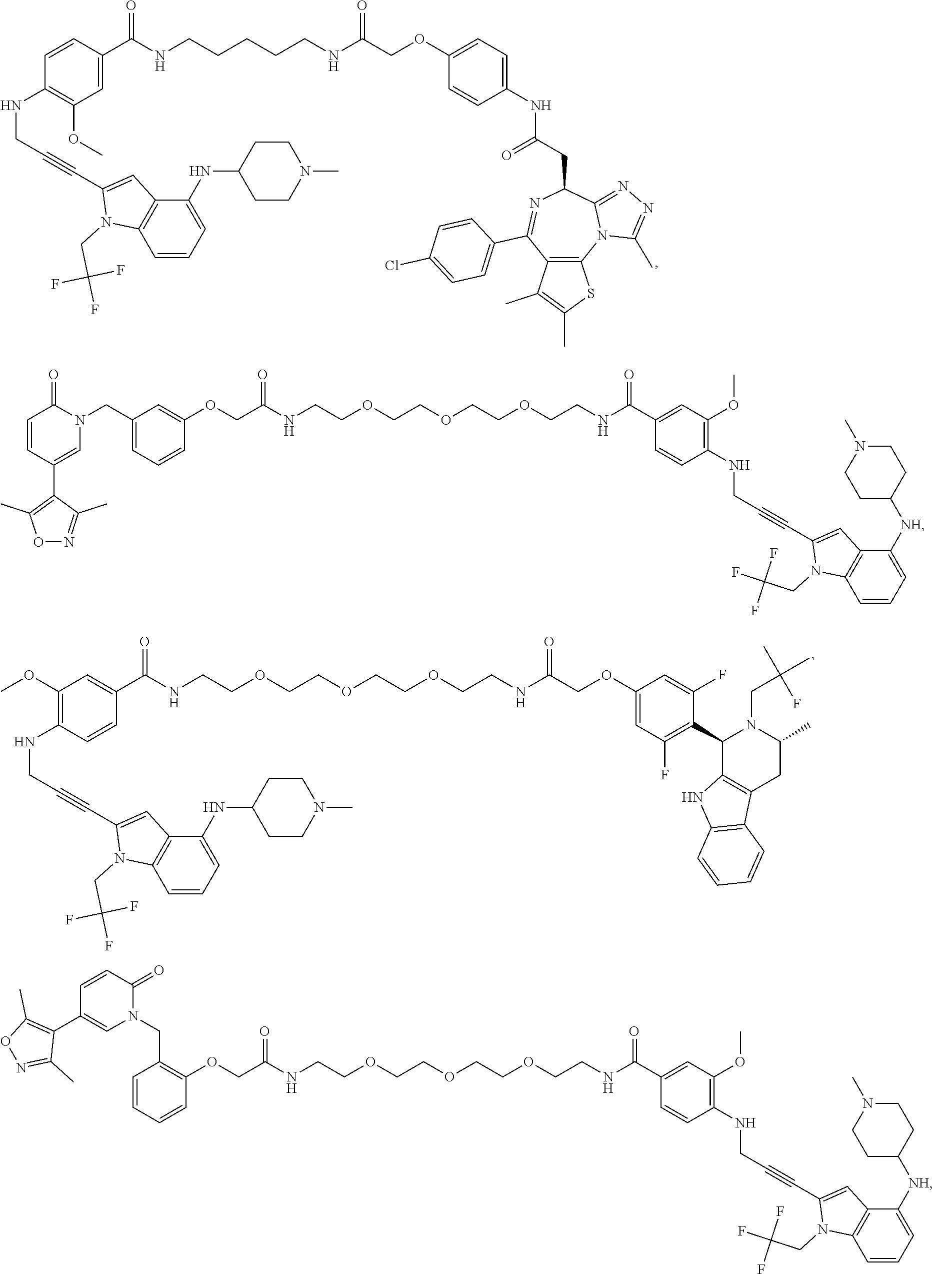

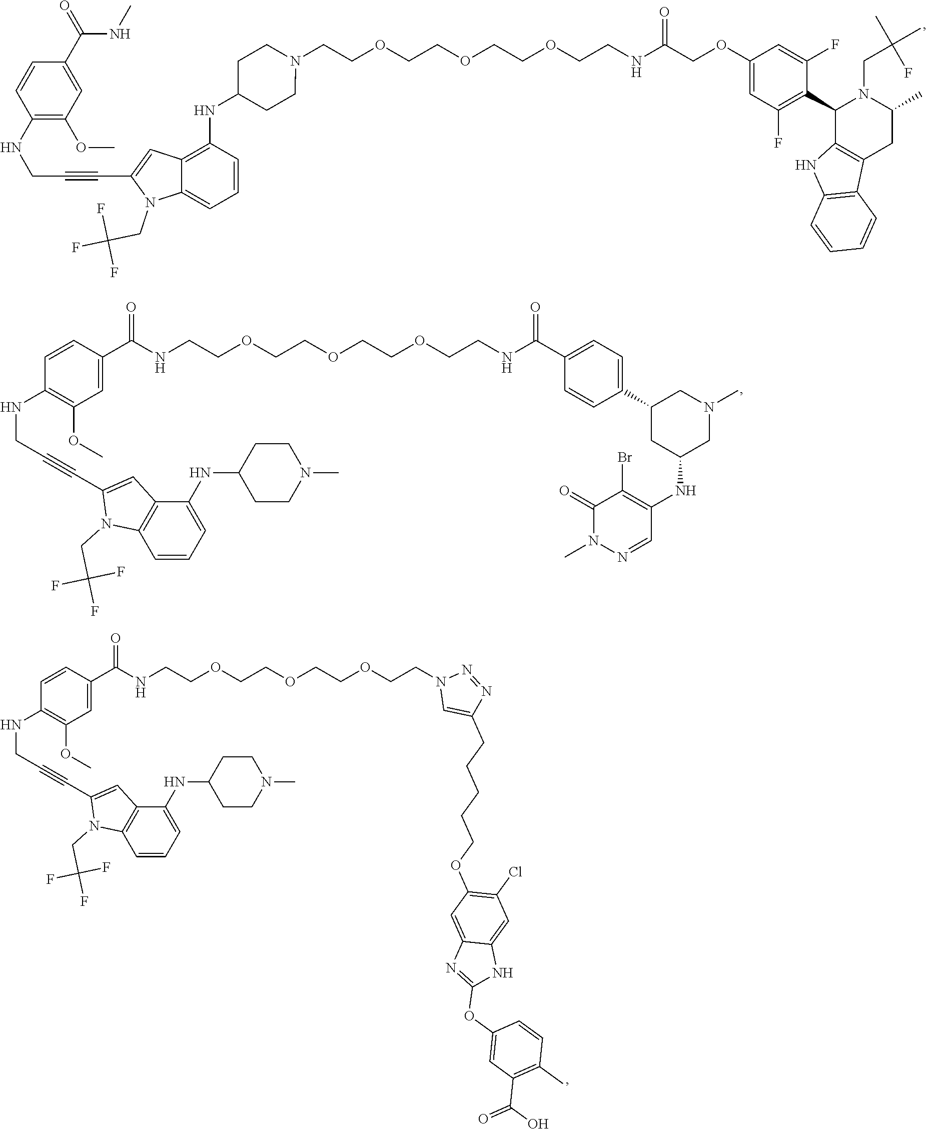

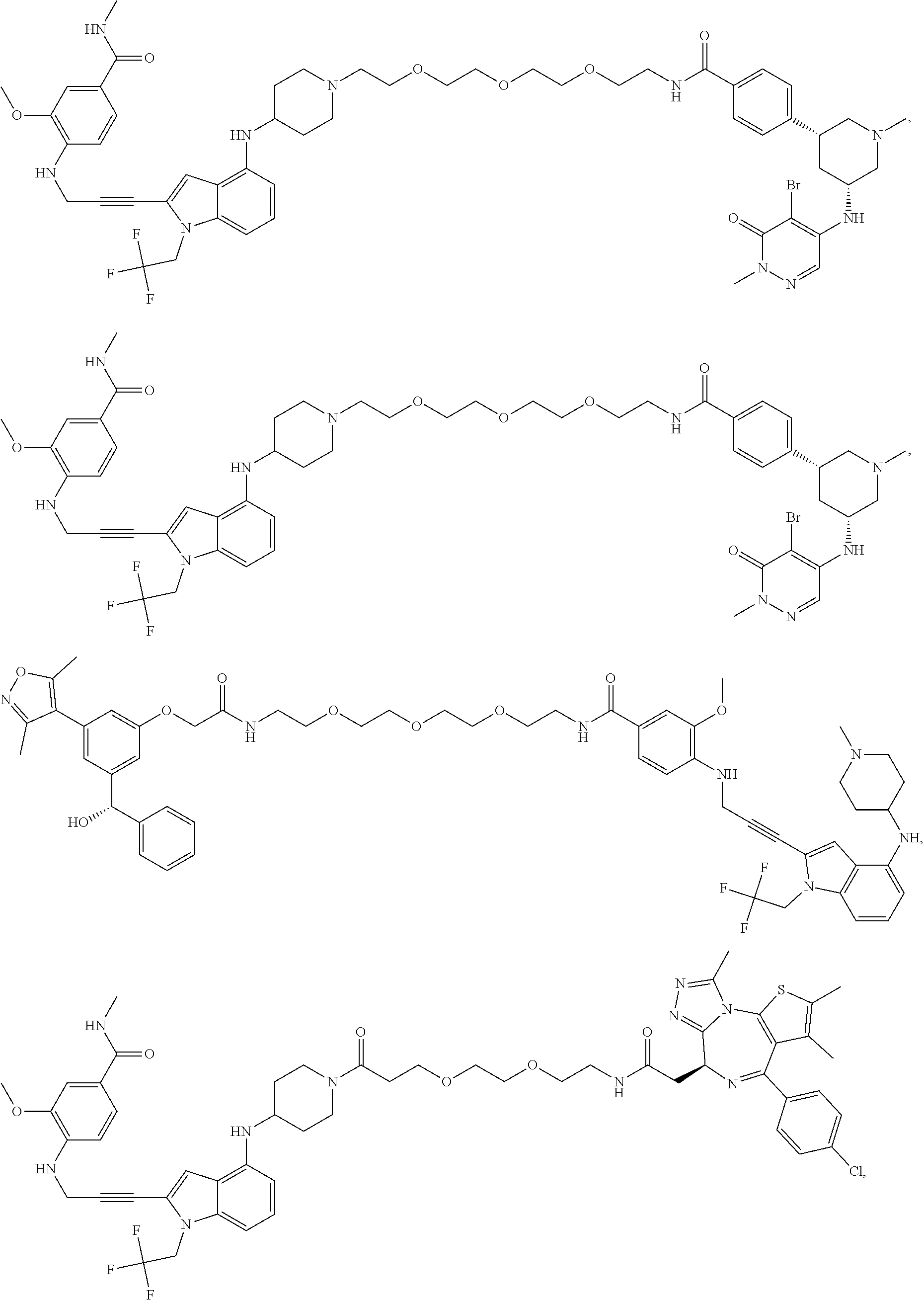

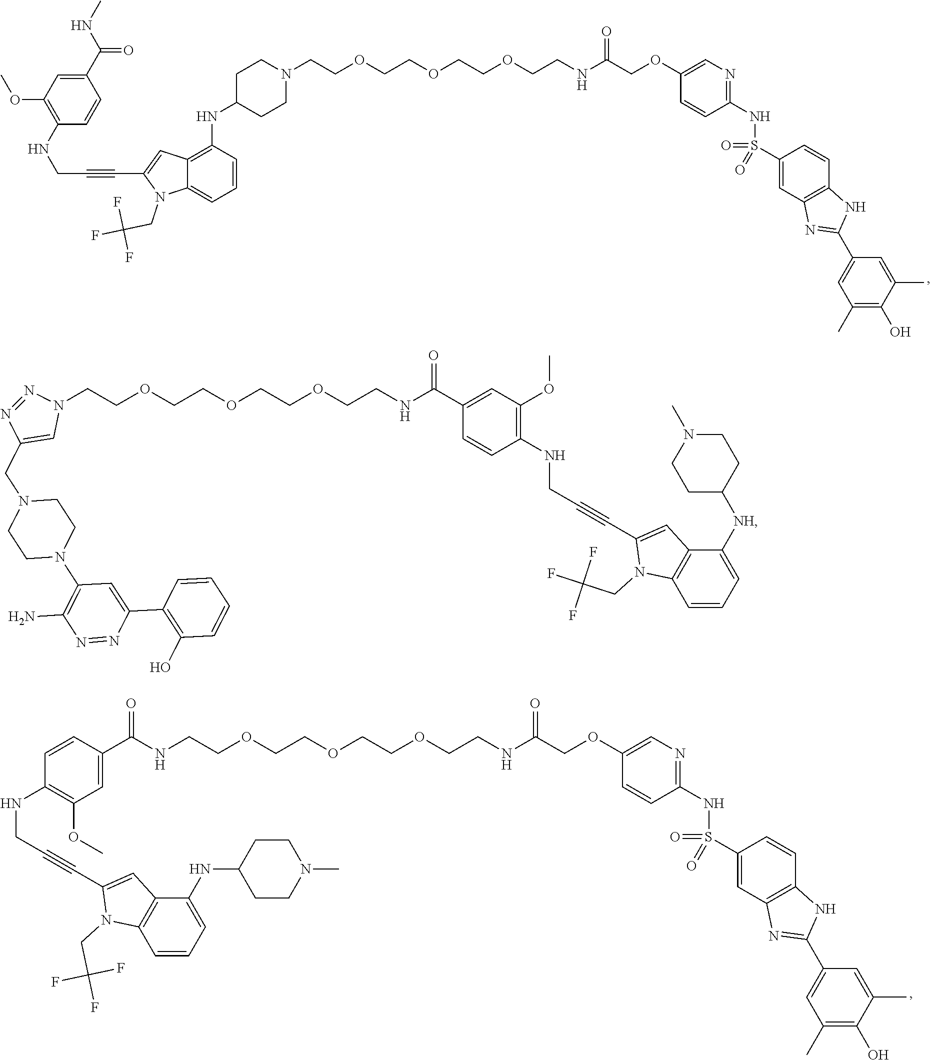

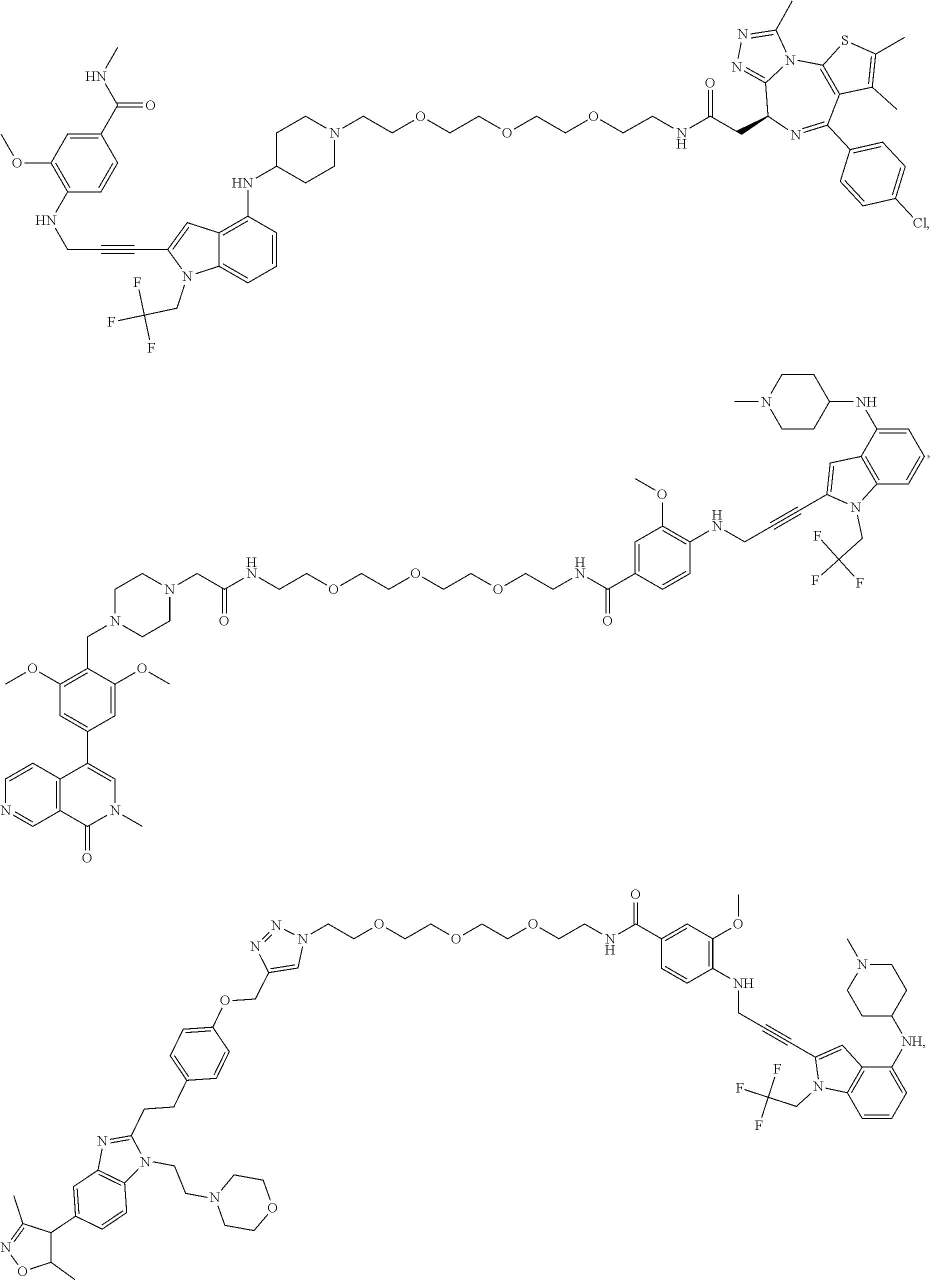 [SMILES: CNC(=O)c1ccc(NCC#Cc2cc3c(NC4CCN(CCOCCOCCOCCNC(=O)C[C@@H]5N=C(c6ccc(Cl)cc6)c6c(sc(C)c6C)-n6c(C)nnc65)CC4)cccc3n2CC(F)(F)F)c(OC)c1 ; COc1cc(C(=O)NCCOCCOCCOCCNC(=O)CN2CCN(Cc3c(OC)cc(-c4cn(C)c(=O)c5cnccc45)cc3OC)CC2)ccc1NCC#Cc1cc2c(NC3CCN(C)CC3)cccc2n1CC(F)(F)F ; COc1cc(C(=O)NCCOCCOCCOCCn2cc(COc3ccc(CCc4nc5cc(C6C(C)=NOC6C)ccc5n4CCN4CCOCC4)cc3)nn2)ccc1NCC#Cc1cc2c(NC3CCN(C)CC3)cccc2n1CC(F)(F)F]

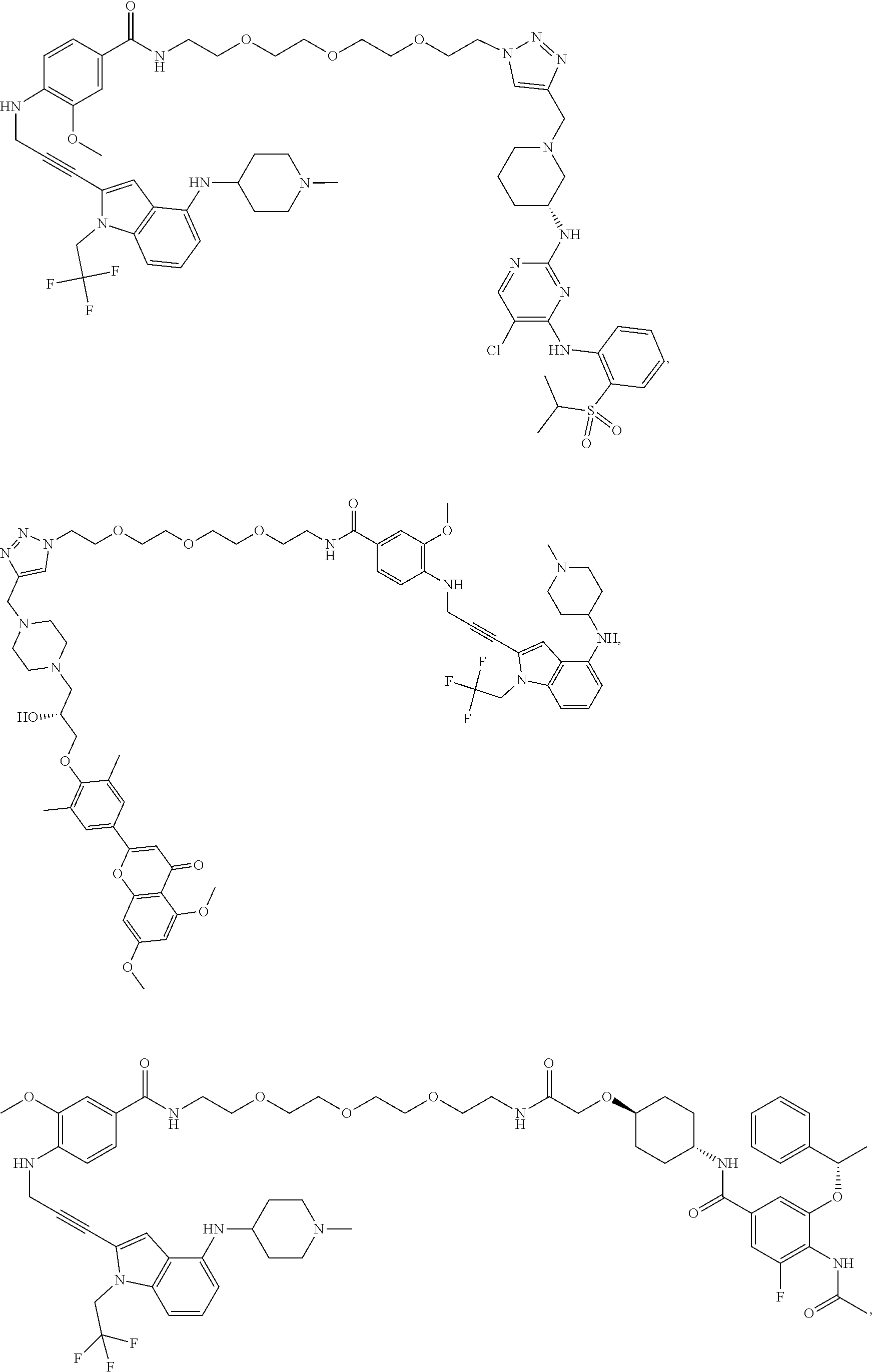 [SMILES: COc1cc(C(=O)NCCOCCOCCOCCn2cc(CN3CCC[C@@H](Nc4ncc(Cl)c(Nc5ccccc5S(=O)(=O)C(C)C)n4)C3)nn2)ccc1NCC#Cc1cc2c(NC3CCN(C)CC3)cccc2n1CC(F)(F)F ; COc1cc(OC)c2c(=O)cc(-c3cc(C)c(OC[C@H](O)CN4CCN(Cc5cn(CCOCCOCCOCCNC(=O)c6ccc(NCC#Cc7cc8c(NC9CCN(C)CC9)cccc8n7CC(F)(F)F)c(OC)c6)nn5)CC4)c(C)c3)oc2c1 ; COc1cc(C(=O)NCCOCCOCCOCCNC(=O)CO[C@H]2CC[C@H](NC(=O)c3cc(F)c(NC(C)=O)c(O[C@@H](C)c4ccccc4)c3)CC2)ccc1NCC#Cc1cc2c(NC3CCN(C)CC3)cccc2n1CC(F)(F)F]

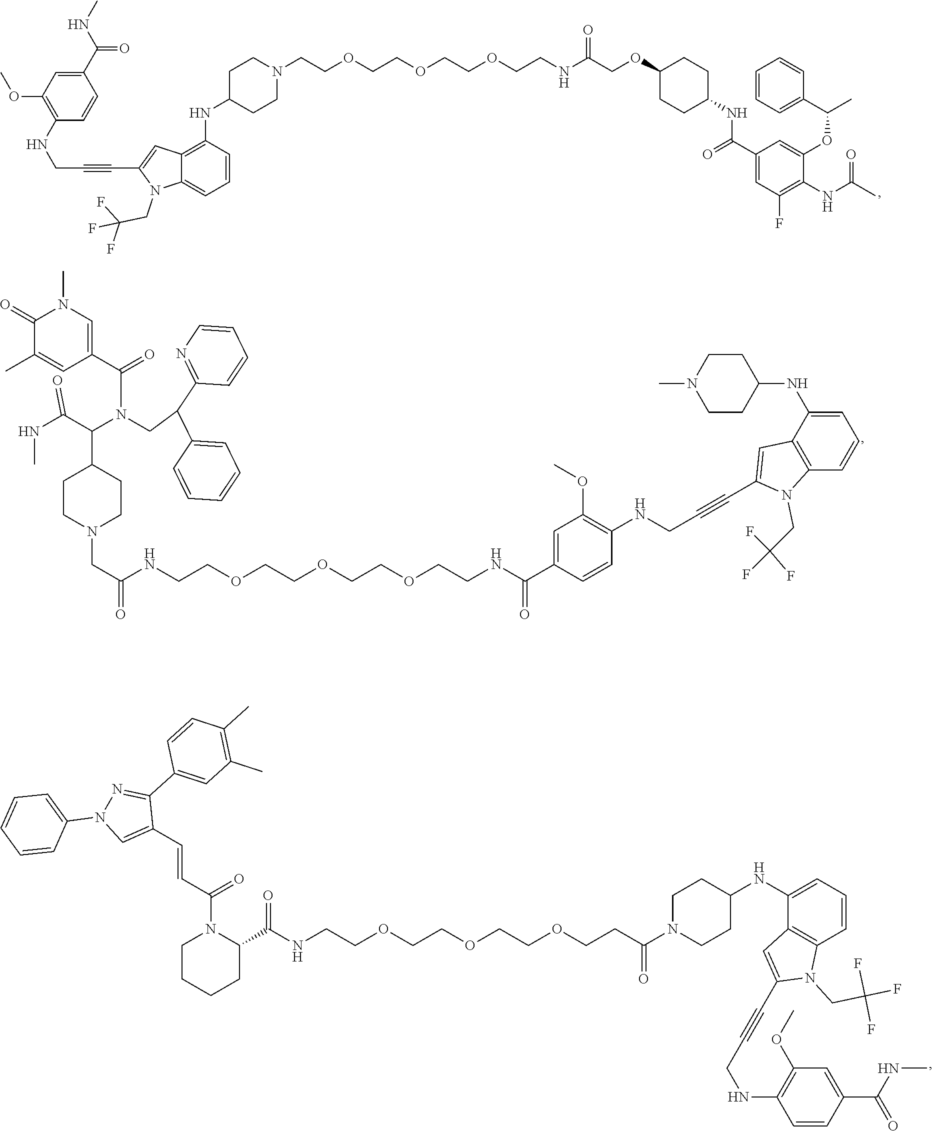

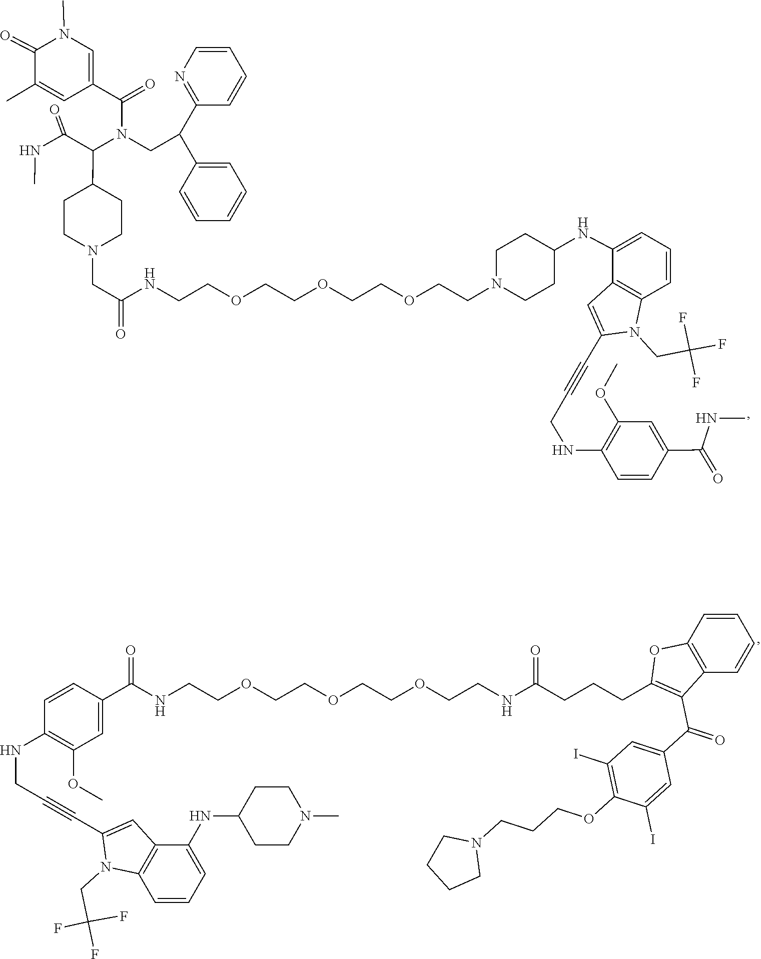 [SMILES: CNC(=O)c1ccc(NCC#Cc2cc3c(NC4CCN(CCOCCOCCOCCNC(=O)CN5CCC(C(C(=O)NC)N(CC(c6ccccc6)c6ccccn6)C(=O)c6cc(C)c(=O)n(C)c6)CC5)CC4)cccc3n2CC(F)(F)F)c(OC)c1 ; COc1cc(C(=O)NCCOCCOCCOCCNC(=O)CCCc2oc3ccccc3c2C(=O)c2cc(I)c(OCCCN3CCCC3)c(I)c2)ccc1NCC#Cc1cc2c(NC3CCN(C)CC3)cccc2n1CC(F)(F)F]

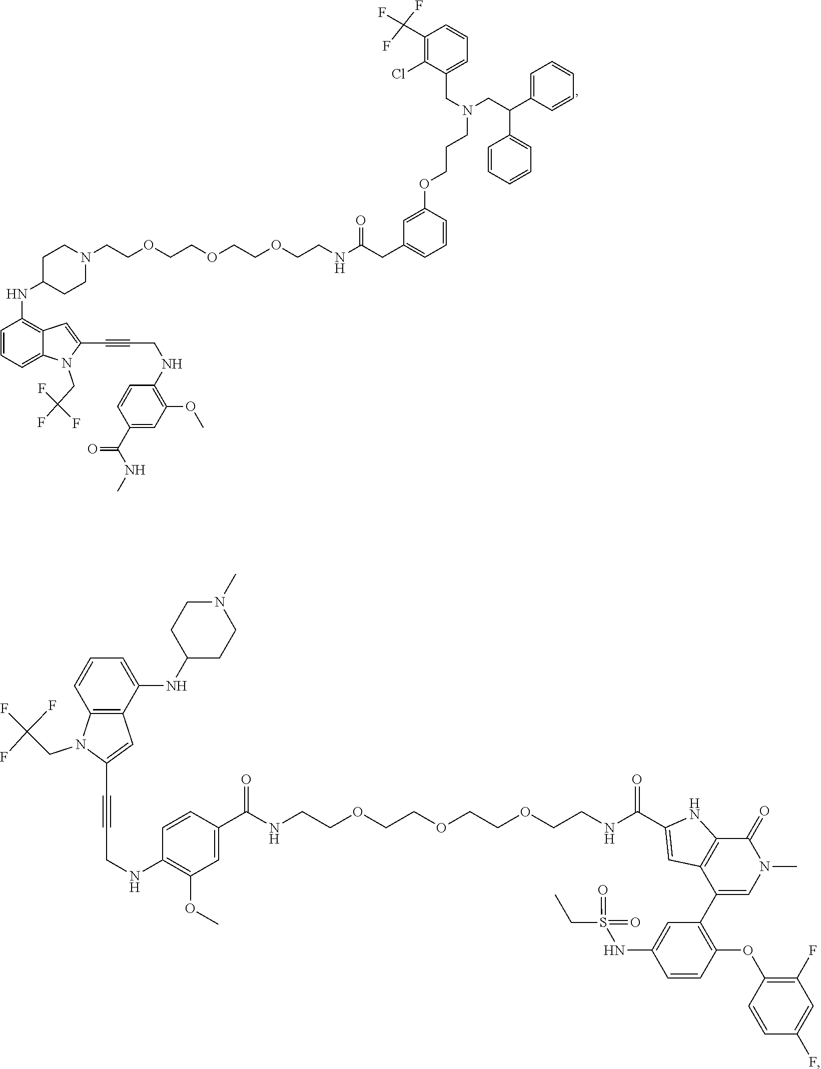 [SMILES: CNC(=O)c1ccc(NCC#Cc2cc3c(NC4CCN(CCOCCOCCOCCNC(=O)Cc5cccc(OCCCN(Cc6cccc(C(F)(F)F)c6Cl)CC(c6ccccc6)c6ccccc6)c5)CC4)cccc3n2CC(F)(F)F)c(OC)c1 ; CCS(=O)(=O)Nc1ccc(Oc2ccc(F)cc2F)c(-c2cn(C)c(=O)c3[nH]c(C(=O)NCCOCCOCCOCCNC(=O)c4ccc(NCC#Cc5cc6c(NC7CCN(C)CC7)cccc6n5CC(F)(F)F)c(OC)c4)cc23)c1]

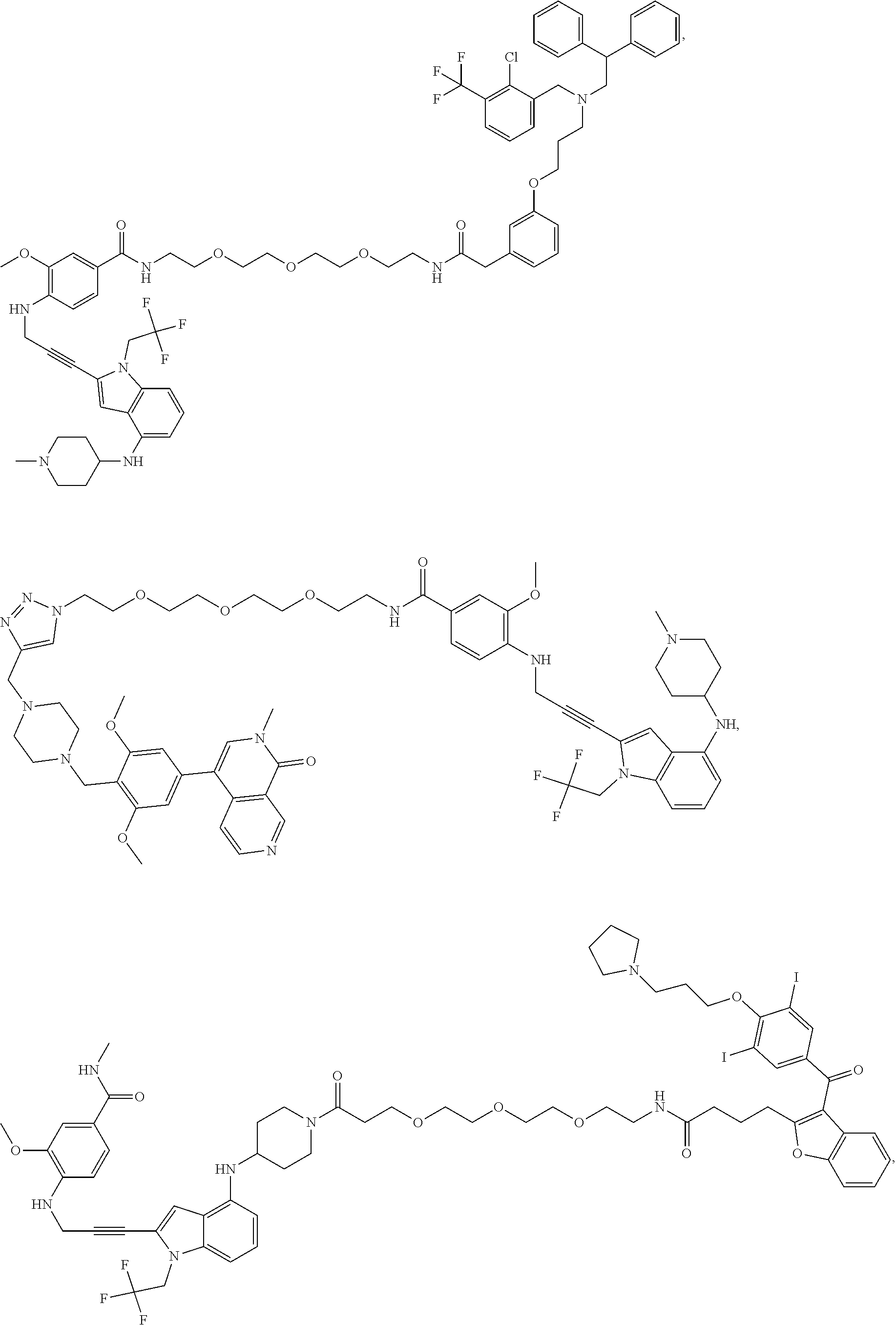 [SMILES: COc1cc(C(=O)NCCOCCOCCOCCNC(=O)Cc2cccc(OCCCN(Cc3cccc(C(F)(F)F)c3Cl)CC(c3ccccc3)c3ccccc3)c2)ccc1NCC#Cc1cc2c(NC3CCN(C)CC3)cccc2n1CC(F)(F)F ; COc1cc(C(=O)NCCOCCOCCOCCn2cc(CN3CCN(Cc4c(OC)cc(-c5cn(C)c(=O)c6cnccc56)cc4OC)CC3)nn2)ccc1NCC#Cc1cc2c(NC3CCN(C)CC3)cccc2n1CC(F)(F)F ; CNC(=O)c1ccc(NCC#Cc2cc3c(NC4CCN(C(=O)CCOCCOCCOCCNC(=O)CCCc5oc6ccccc6c5C(=O)c5cc(I)c(OCCCN6CCCC6)c(I)c5)CC4)cccc3n2CC(F)(F)F)c(OC)c1]

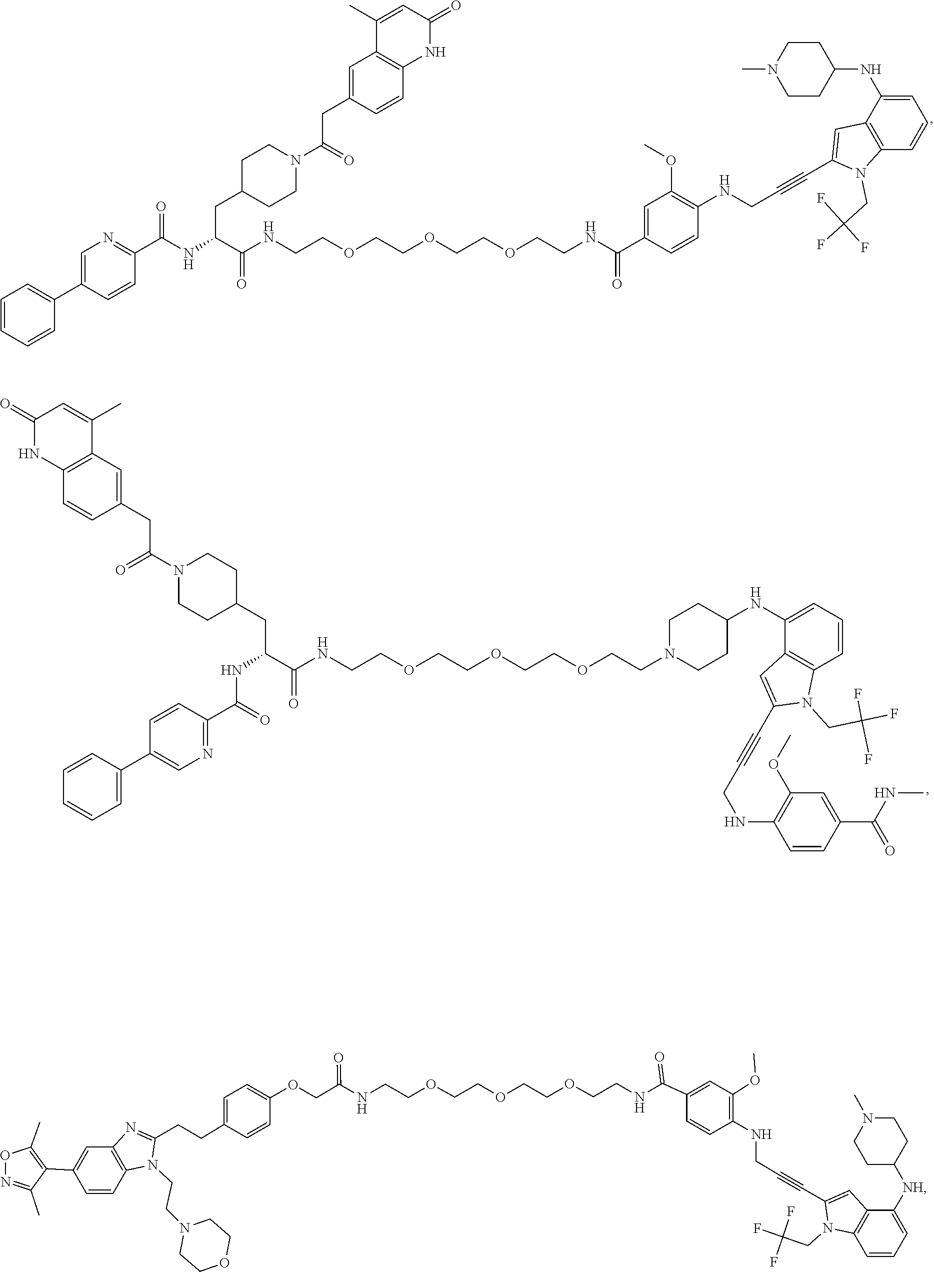 [SMILES: COc1cc(C(=O)NCCOCCOCCOCCNC(=O)[C@@H](CC2CCN(C(=O)Cc3ccc4[nH]c(=O)cc(C)c4c3)CC2)NC(=O)c2ccc(-c3ccccc3)cn2)ccc1NCC#Cc1cc2c(NC3CCN(C)CC3)cccc2n1CC(F)(F)F ; CNC(=O)c1ccc(NCC#Cc2cc3c(NC4CCN(CCOCCOCCOCCNC(=O)[C@@H](CC5CCN(C(=O)Cc6ccc7[nH]c(=O)cc(C)c7c6)CC5)NC(=O)c5ccc(-c6ccccc6)cn5)CC4)cccc3n2CC(F)(F)F)c(OC)c1 ; COc1cc(C(=O)NCCOCCOCCOCCNC(=O)COc2ccc(CCc3nc4cc(-c5c(C)noc5C)ccc4n3CCN3CCOCC3)cc2)ccc1NCC#Cc1cc2c(NC3CCN(C)CC3)cccc2n1CC(F)(F)F]

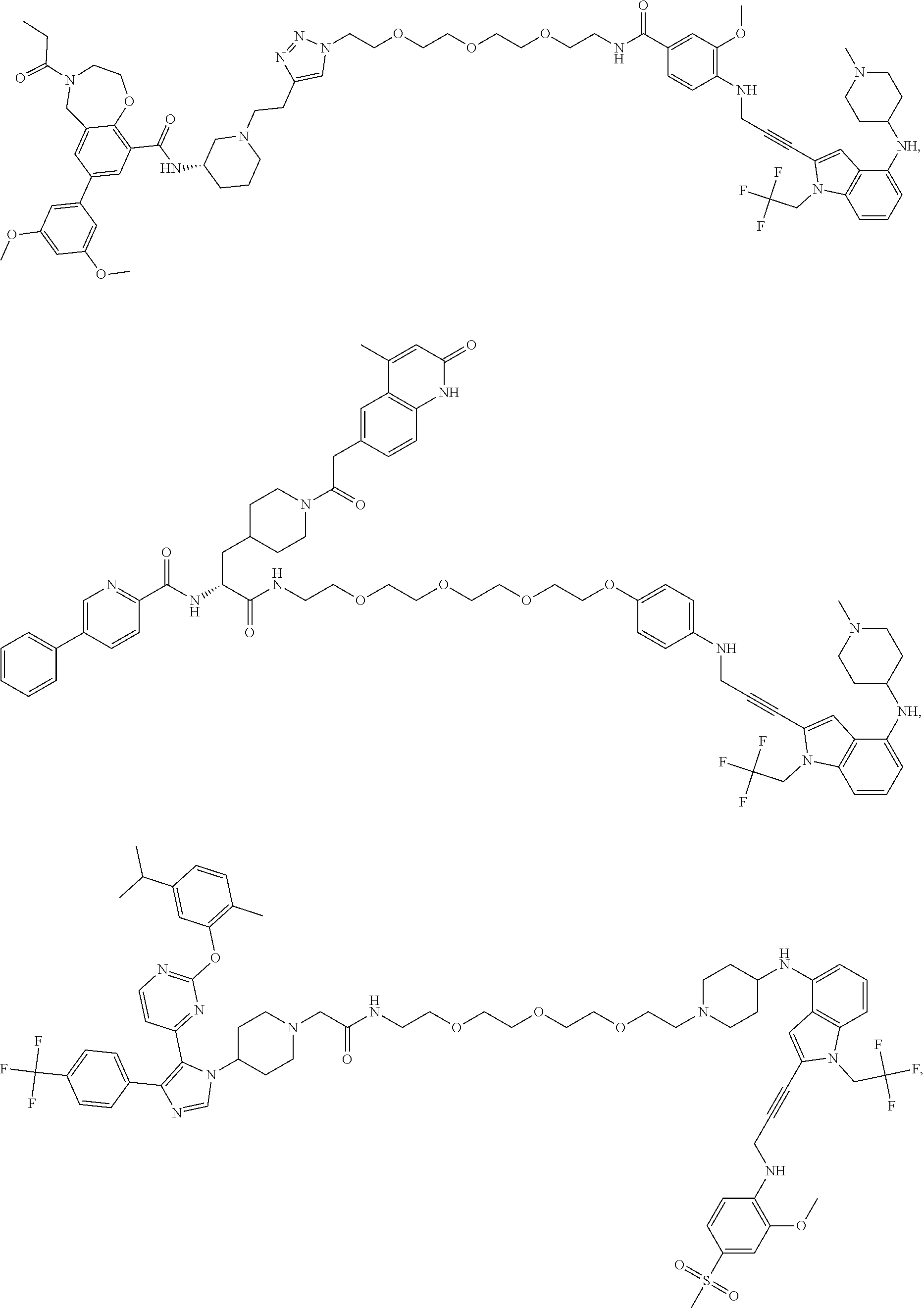 [SMILES: CCC(=O)N1CCOc2c(cc(-c3cc(OC)cc(OC)c3)cc2C(=O)N[C@H]2CCCN(CCc3cn(CCOCCOCCOCCNC(=O)c4ccc(NCC#Cc5cc6c(NC7CCN(C)CC7)cccc6n5CC(F)(F)F)c(OC)c4)nn3)C2)C1 ; Cc1cc(=O)[nH]c2ccc(CC(=O)N3CCC(C[C@@H](NC(=O)c4ccc(-c5ccccc5)cn4)C(=O)NCCOCCOCCOCCOc4ccc(NCC#Cc5cc6c(NC7CCN(C)CC7)cccc6n5CC(F)(F)F)cc4)CC3)cc12 ; COc1cc(S(C)(=O)=O)ccc1NCC#Cc1cc2c(NC3CCN(CCOCCOCCOCCNC(=O)CN4CCC(n5cnc(-c6ccc(C(F)(F)F)cc6)c5-c5ccnc(Oc6cc(C(C)C)ccc6C)n5)CC4)CC3)cccc2n1CC(F)(F)F]

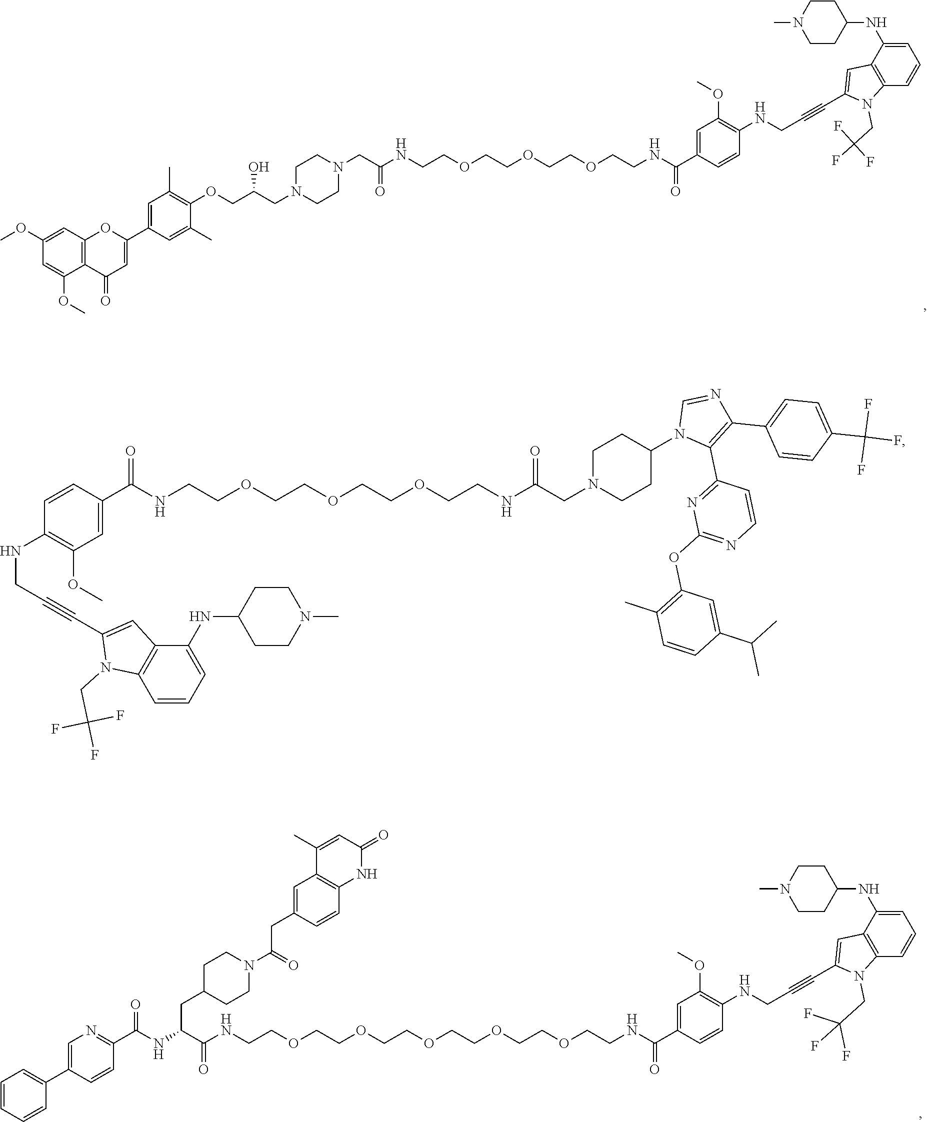 [SMILES: COc1cc(OC)c2c(=O)cc(-c3cc(C)c(OC[C@H](O)CN4CCN(CC(=O)NCCOCCOCCOCCNC(=O)c5ccc(NCC#Cc6cc7c(NC8CCN(C)CC8)cccc7n6CC(F)(F)F)c(OC)c5)CC4)c(C)c3)oc2c1 ; COc1cc(C(=O)NCCOCCOCCOCCNC(=O)CN2CCC(n3cnc(-c4ccc(C(F)(F)F)cc4)c3-c3ccnc(Oc4cc(C(C)C)ccc4C)n3)CC2)ccc1NCC#Cc1cc2c(NC3CCN(C)CC3)cccc2n1CC(F)(F)F ; COc1cc(C(=O)NCCOCCOCCOCCOCCOCCNC(=O)[C@@H](CC2CCN(C(=O)Cc3ccc4[nH]c(=O)cc(C)c4c3)CC2)NC(=O)c2ccc(-c3ccccc3)cn2)ccc1NCC#Cc1cc2c(NC3CCN(C)CC3)cccc2n1CC(F)(F)F]

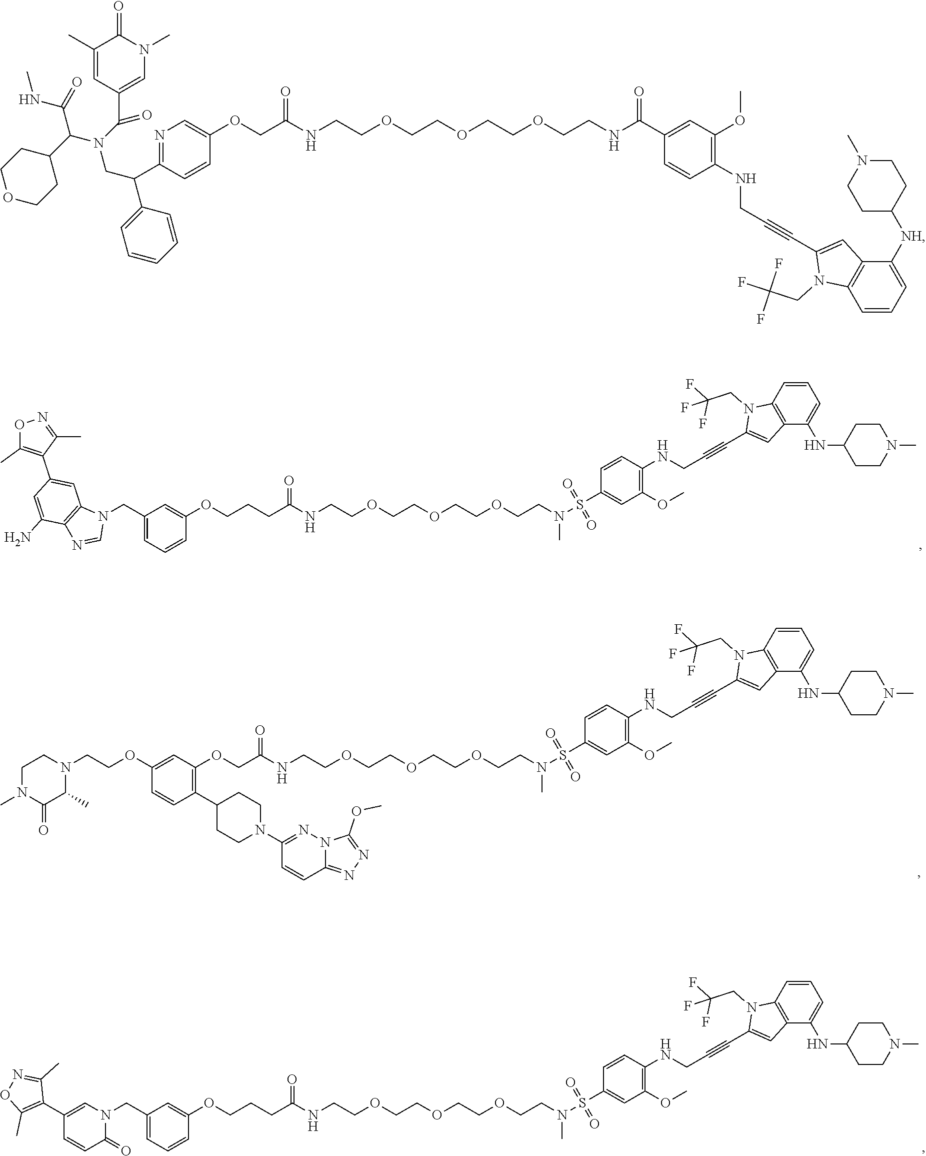

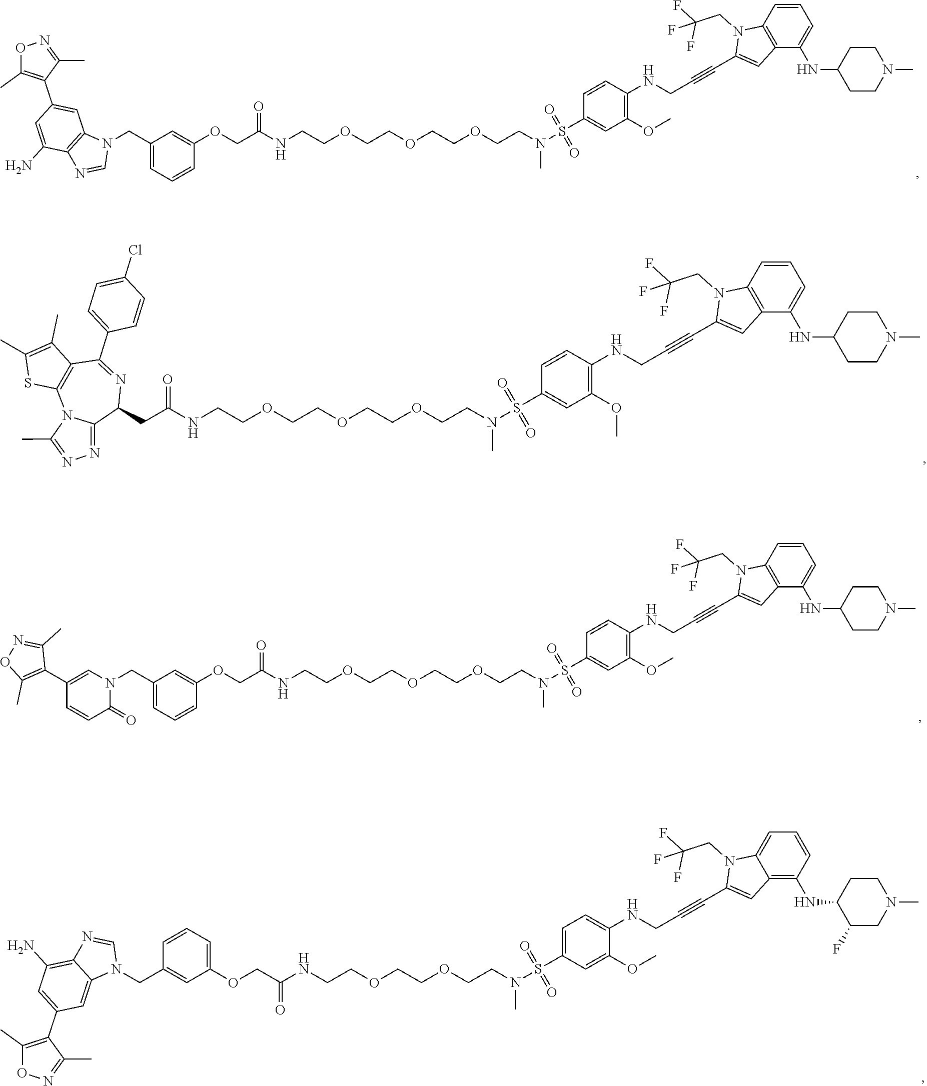

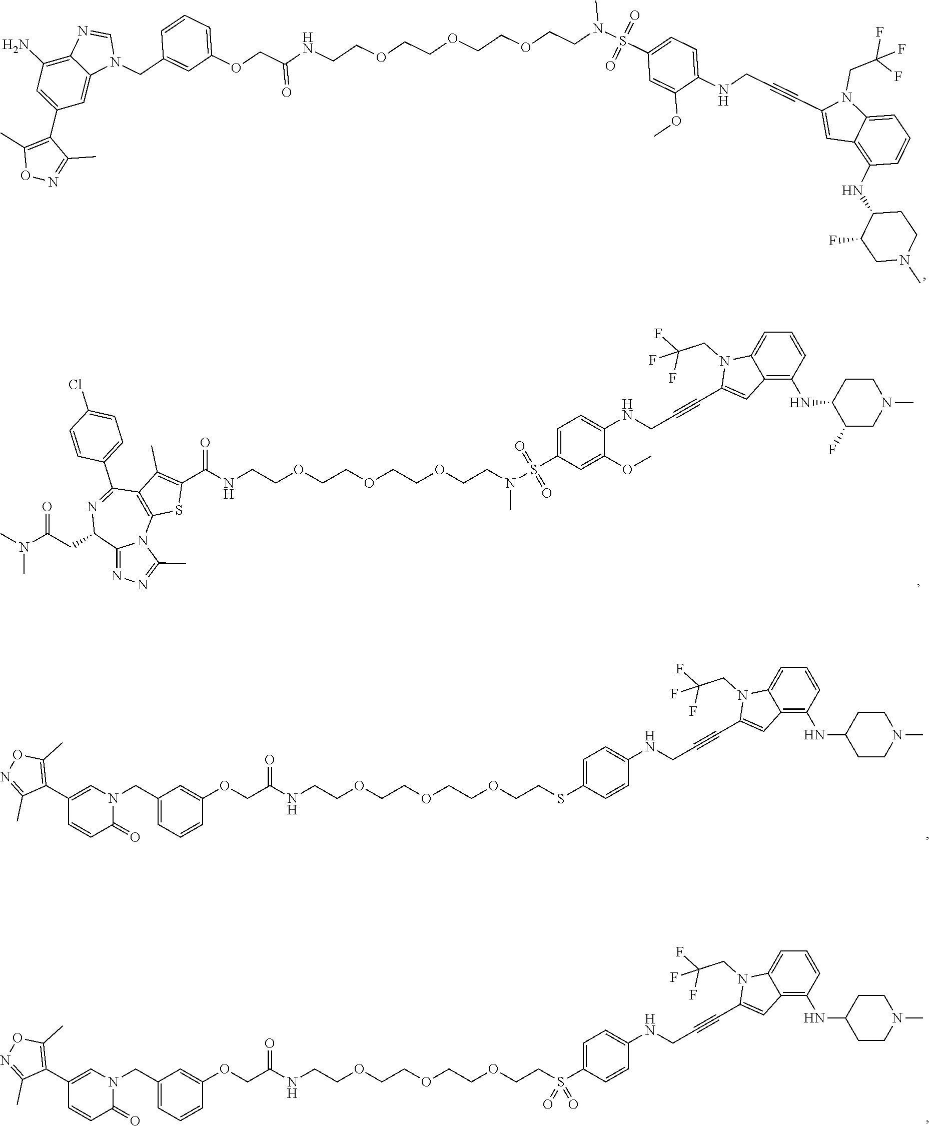

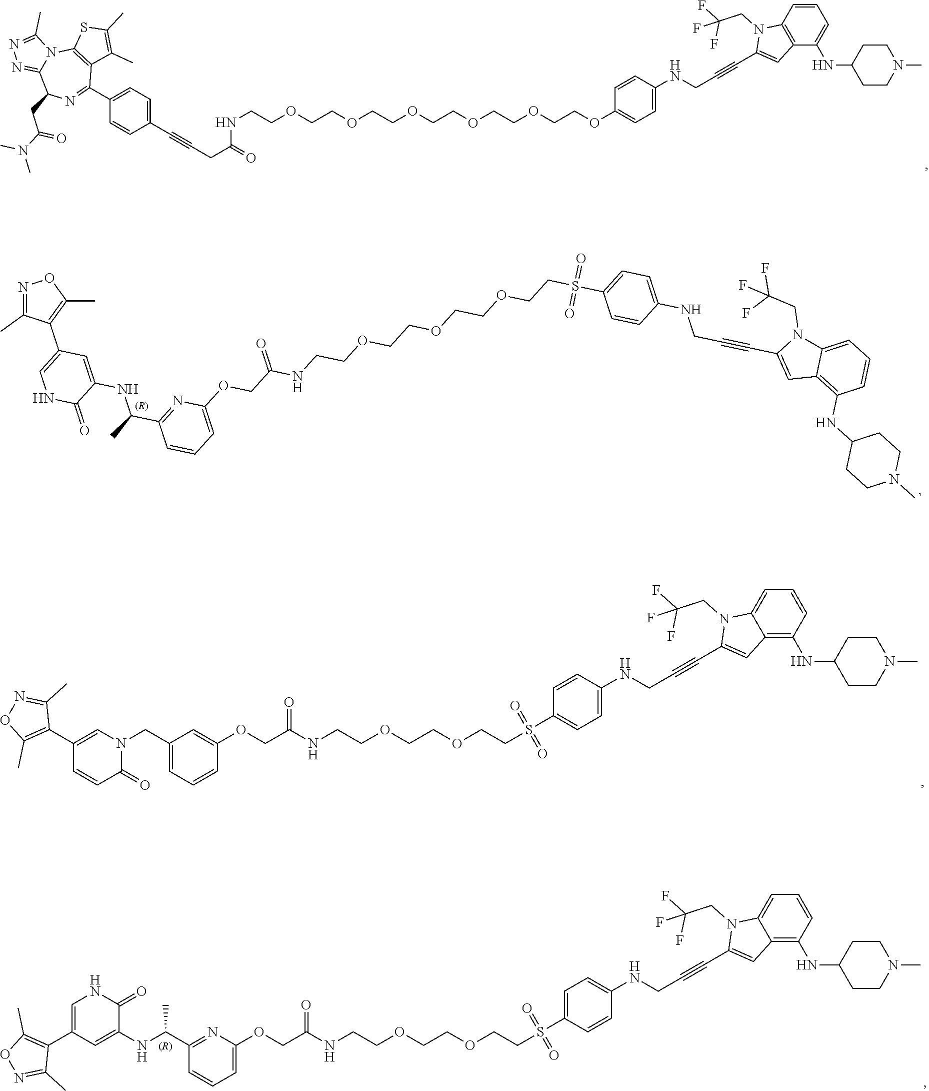

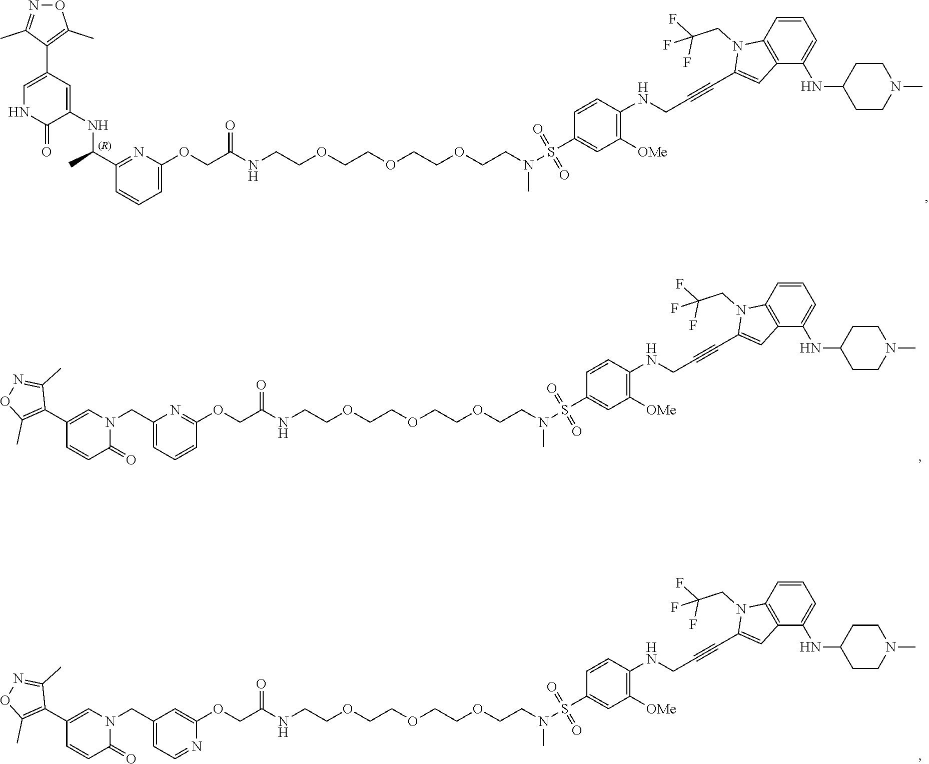

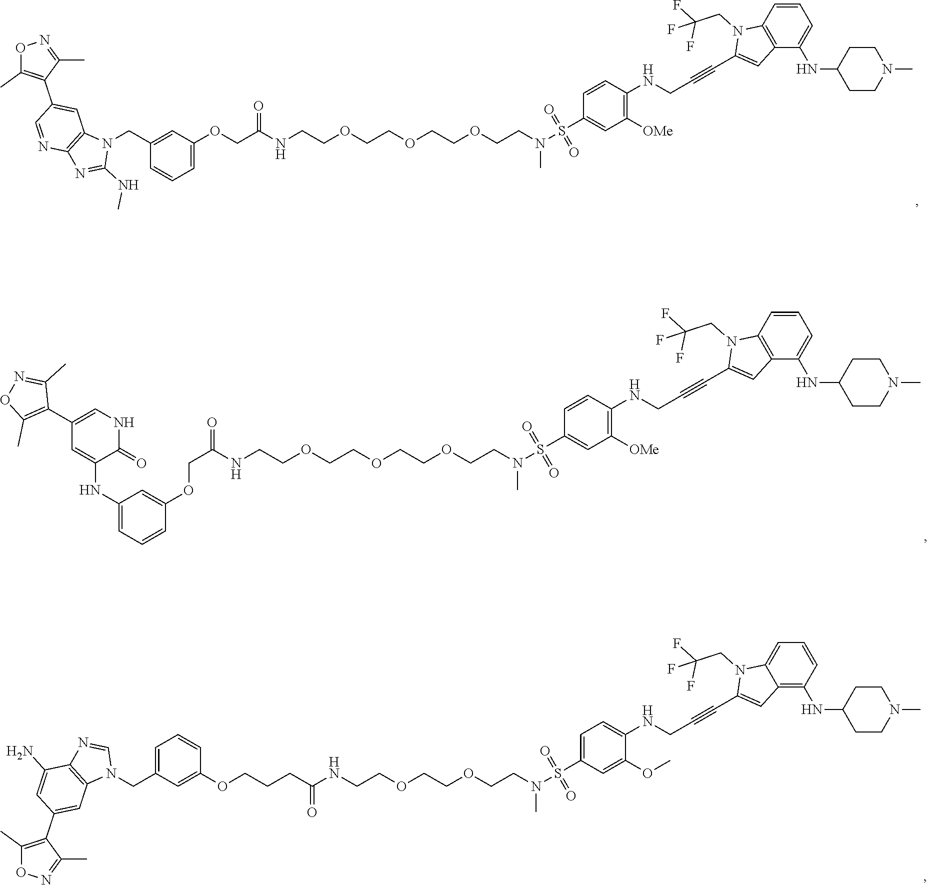

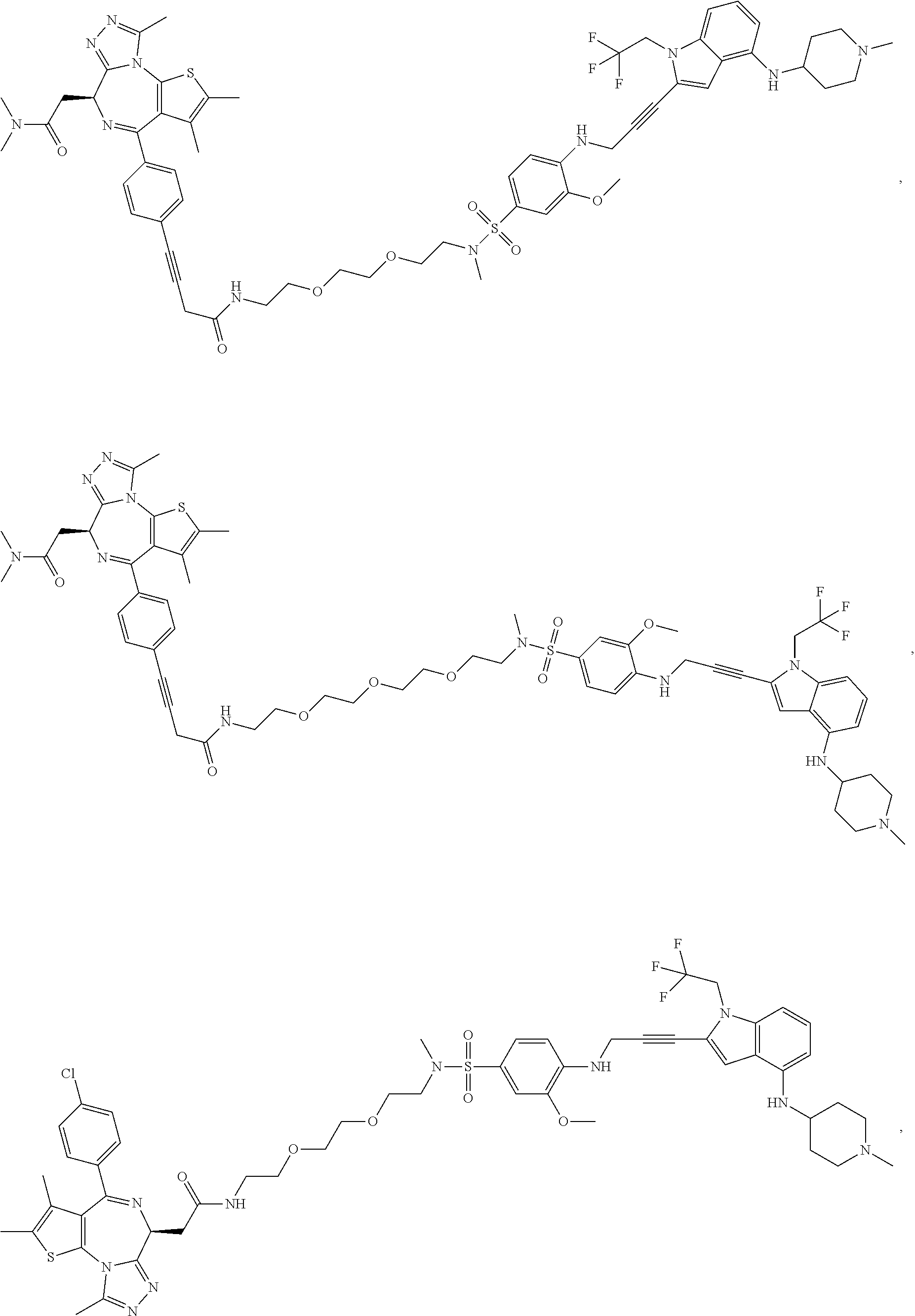

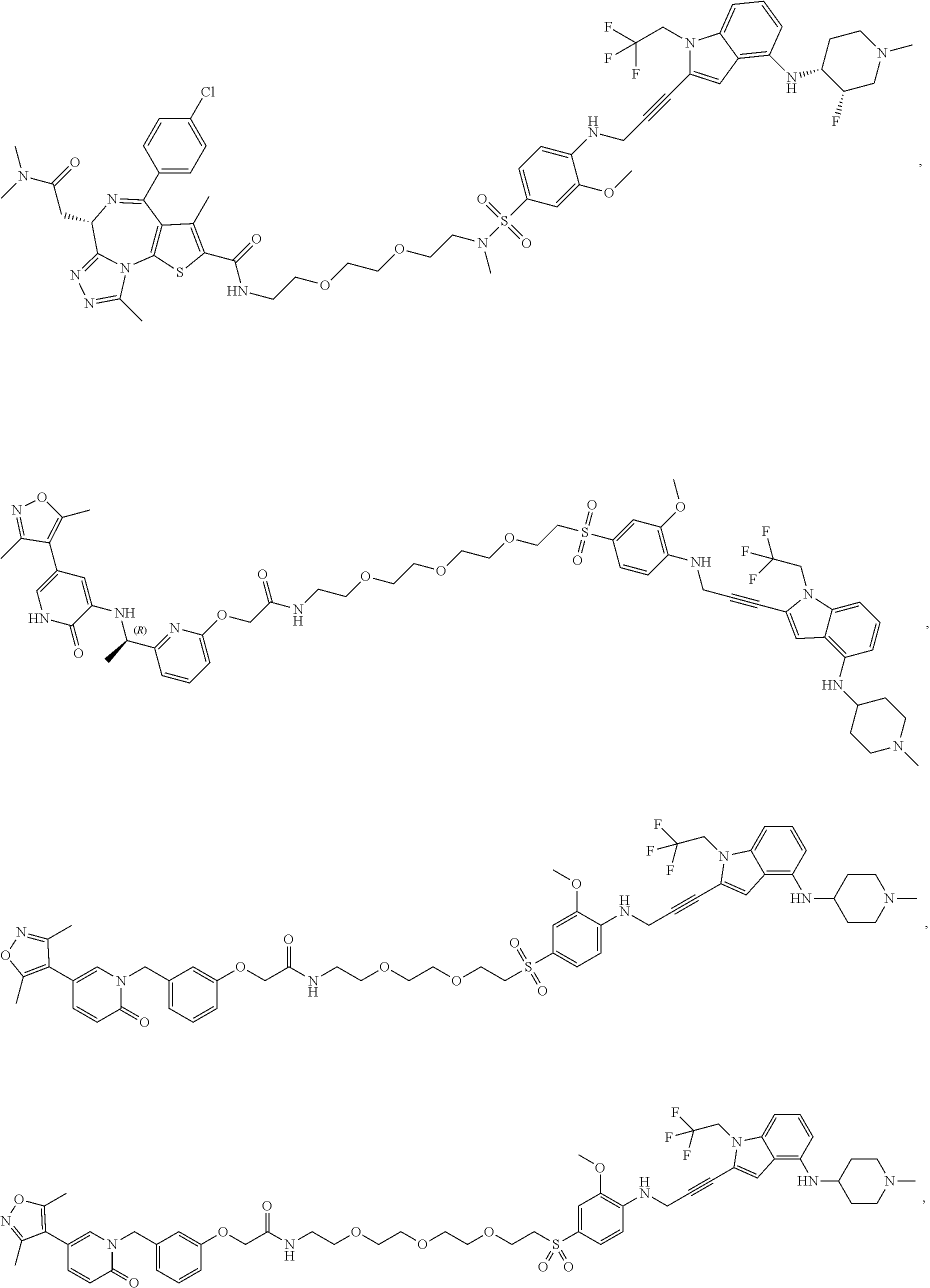

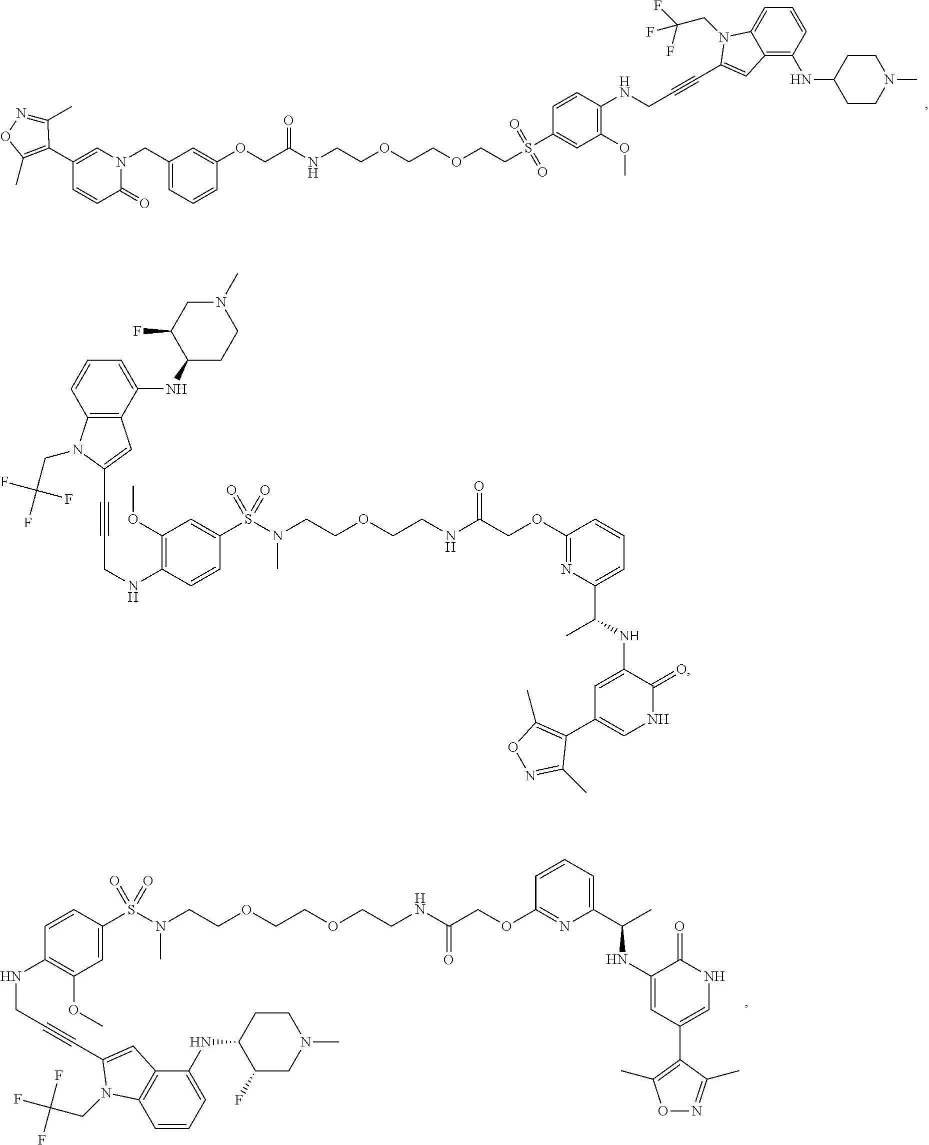 [SMILES: COc1cc(S(=O)(=O)CCOCCOCCNC(=O)COc2cccc(Cn3cc(-c4c(C)noc4C)ccc3=O)c2)ccc1NCC#Cc1cc2c(NC3CCN(C)CC3)cccc2n1CC(F)(F)F ; COc1cc(S(=O)(=O)N(C)CCOCCNC(=O)COc2cccc([C@@H](C)Nc3cc(-c4c(C)noc4C)c[nH]c3=O)n2)ccc1NCC#Cc1cc2c(N[C@@H]3CCN(C)C[C@@H]3F)cccc2n1CC(F)(F)F ; COc1cc(S(=O)(=O)N(C)CCOCCOCCNC(=O)COc2cccc([C@@H](C)Nc3cc(-c4c(C)noc4C)c[nH]c3=O)n2)ccc1NCC#Cc1cc2c(N[C@@H]3CCN(C)C[C@@H]3F)cccc2n1CC(F)(F)F]

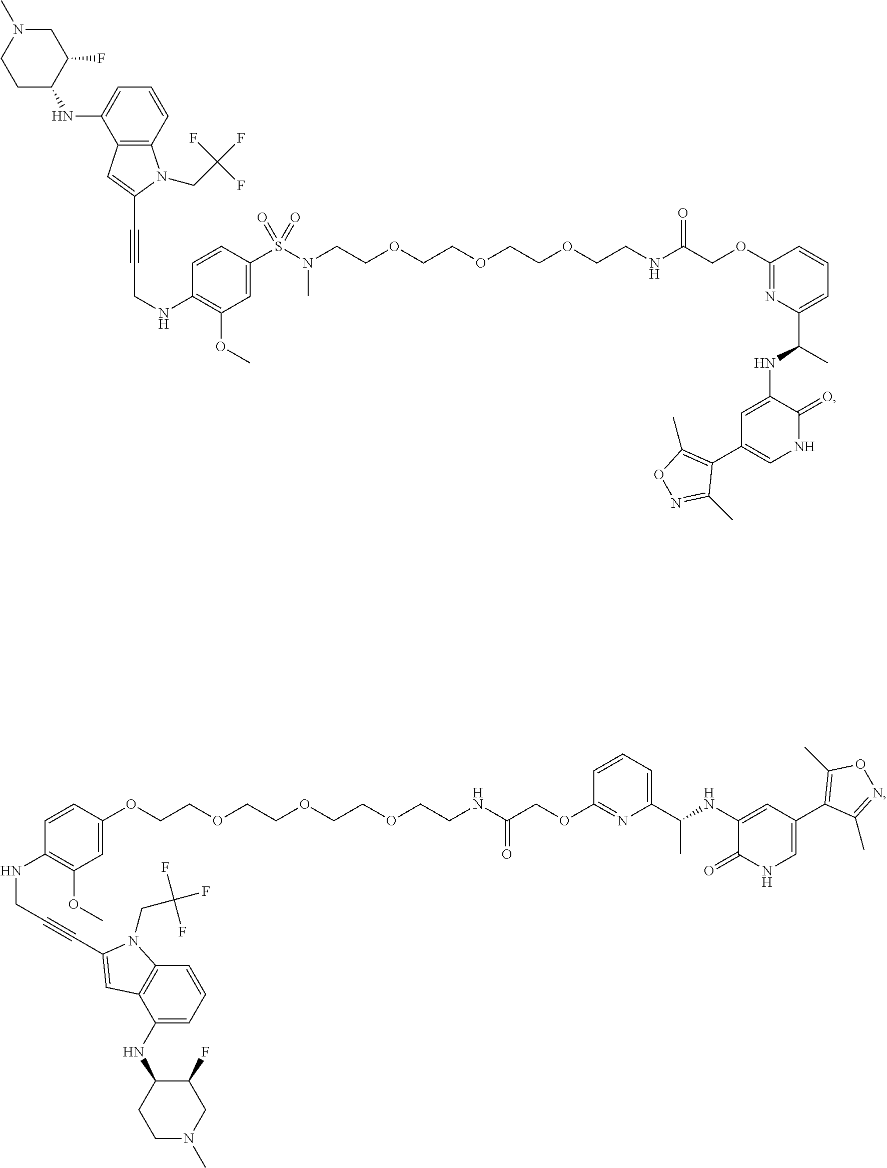 [SMILES: COc1cc(S(=O)(=O)N(C)CCOCCOCCOCCNC(=O)COc2cccc([C@@H](C)Nc3cc(-c4c(C)noc4C)c[nH]c3=O)n2)ccc1NCC#Cc1cc2c(N[C@@H]3CCN(C)C[C@@H]3F)cccc2n1CC(F)(F)F ; COc1cc(OCCOCCOCCOCCNC(=O)COc2cccc([C@@H](C)Nc3cc(-c4c(C)noc4C)c[nH]c3=O)n2)ccc1NCC#Cc1cc2c(N[C@@H]3CCN(C)C[C@@H]3F)cccc2n1CC(F)(F)F]

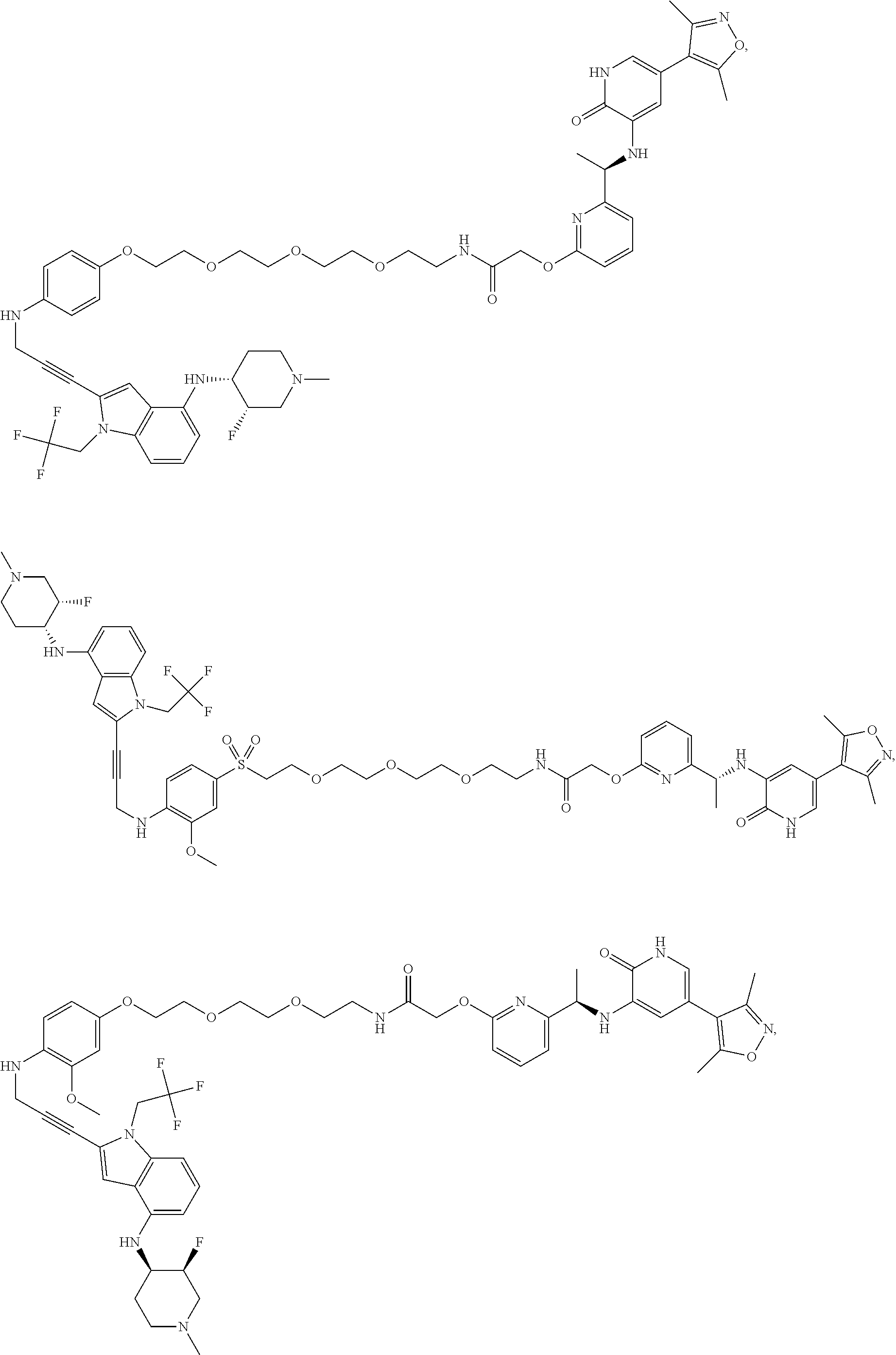 [SMILES: Cc1noc(C)c1-c1c[nH]c(=O)c(N[C@H](C)c2cccc(OCC(=O)NCCOCCOCCOCCOc3ccc(NCC#Cc4cc5c(N[C@@H]6CCN(C)C[C@@H]6F)cccc5n4CC(F)(F)F)cc3)n2)c1 ; COc1cc(S(=O)(=O)CCOCCOCCOCCNC(=O)COc2cccc([C@@H](C)Nc3cc(-c4c(C)noc4C)c[nH]c3=O)n2)ccc1NCC#Cc1cc2c(N[C@@H]3CCN(C)C[C@@H]3F)cccc2n1CC(F)(F)F ; COc1cc(OCCOCCOCCNC(=O)COc2cccc([C@@H](C)Nc3cc(-c4c(C)noc4C)c[nH]c3=O)n2)ccc1NCC#Cc1cc2c(N[C@@H]3CCN(C)C[C@@H]3F)cccc2n1CC(F)(F)F]

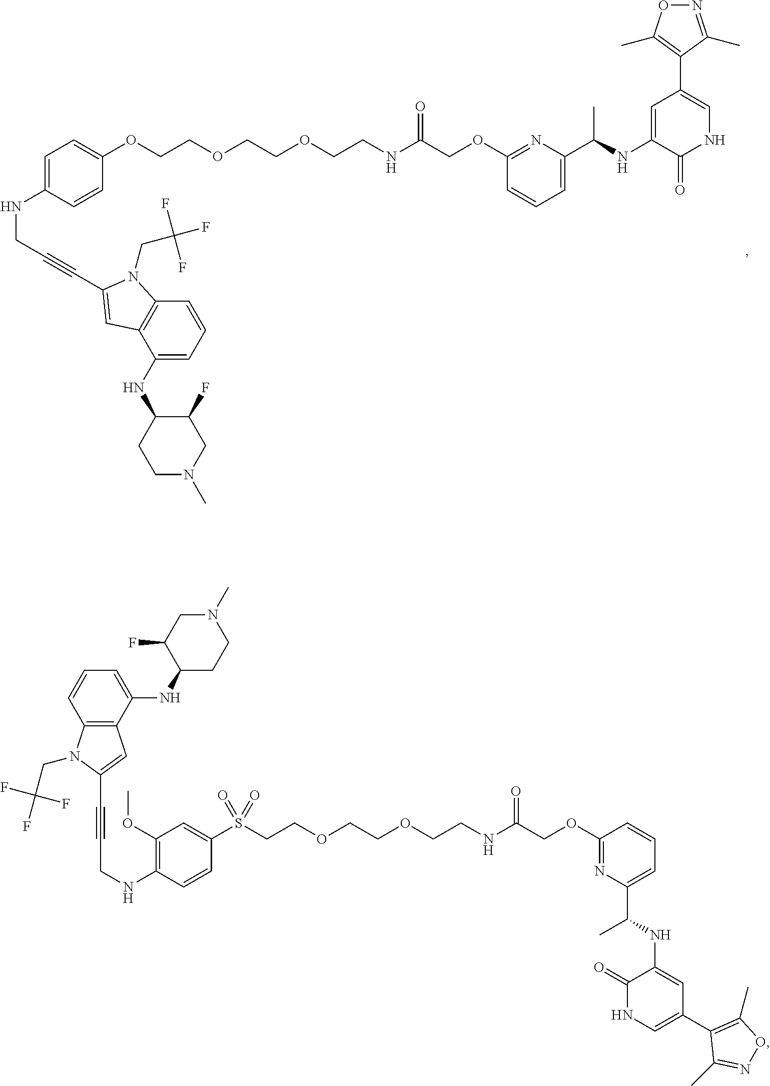 [SMILES: Cc1noc(C)c1-c1c[nH]c(=O)c(N[C@H](C)c2cccc(OCC(=O)NCCOCCOCCOc3ccc(NCC#Cc4cc5c(N[C@@H]6CCN(C)C[C@@H]6F)cccc5n4CC(F)(F)F)cc3)n2)c1 ; COc1cc(S(=O)(=O)CCOCCOCCNC(=O)COc2cccc([C@@H](C)Nc3cc(-c4c(C)noc4C)c[nH]c3=O)n2)ccc1NCC#Cc1cc2c(N[C@@H]3CCN(C)C[C@@H]3F)cccc2n1CC(F)(F)F]

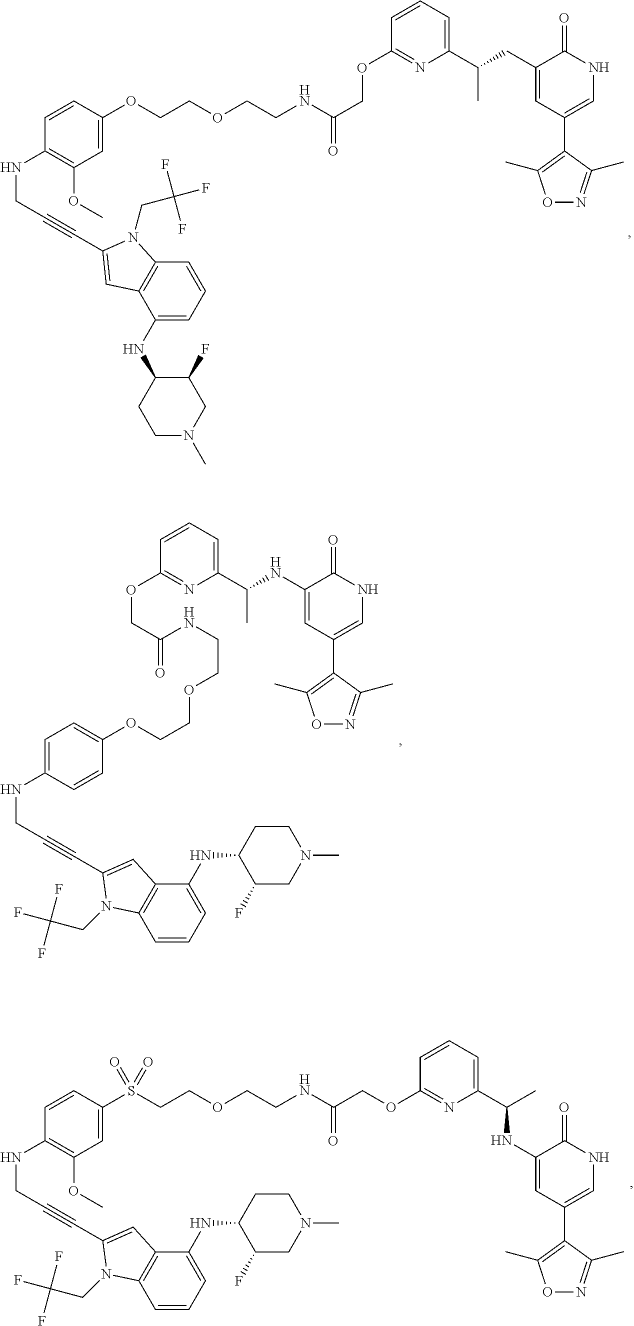 [SMILES: COc1cc(OCCOCCNC(=O)COc2cccc([C@@H](C)Cc3cc(-c4c(C)noc4C)c[nH]c3=O)n2)ccc1NCC#Cc1cc2c(N[C@@H]3CCN(C)C[C@@H]3F)cccc2n1CC(F)(F)F ; Cc1noc(C)c1-c1c[nH]c(=O)c(N[C@H](C)c2cccc(OCC(=O)NCCOCCOc3ccc(NCC#Cc4cc5c(N[C@@H]6CCN(C)C[C@@H]6F)cccc5n4CC(F)(F)F)cc3)n2)c1 ; COc1cc(S(=O)(=O)CCOCCNC(=O)COc2cccc([C@@H](C)Nc3cc(-c4c(C)noc4C)c[nH]c3=O)n2)ccc1NCC#Cc1cc2c(N[C@@H]3CCN(C)C[C@@H]3F)cccc2n1CC(F)(F)F]

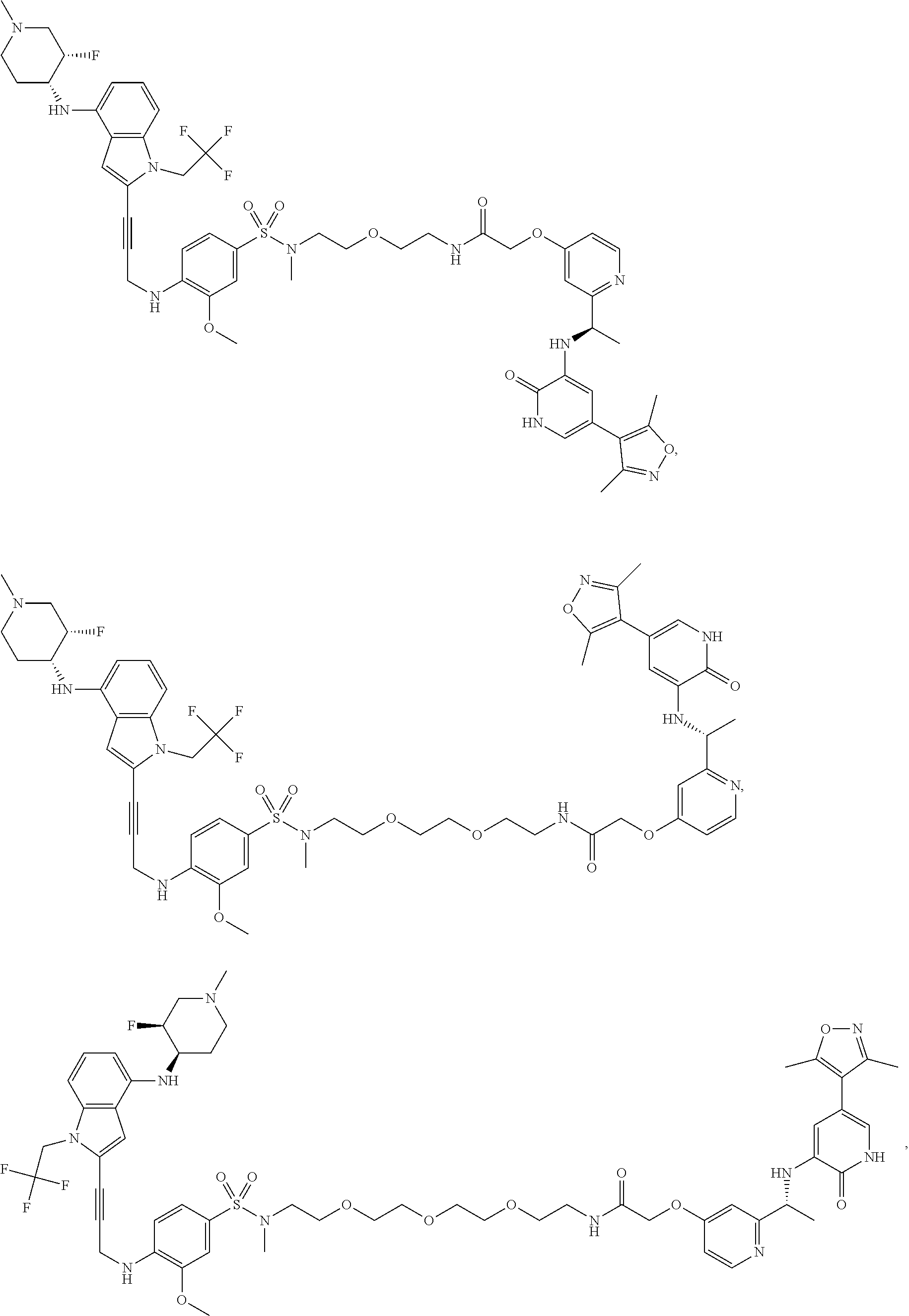

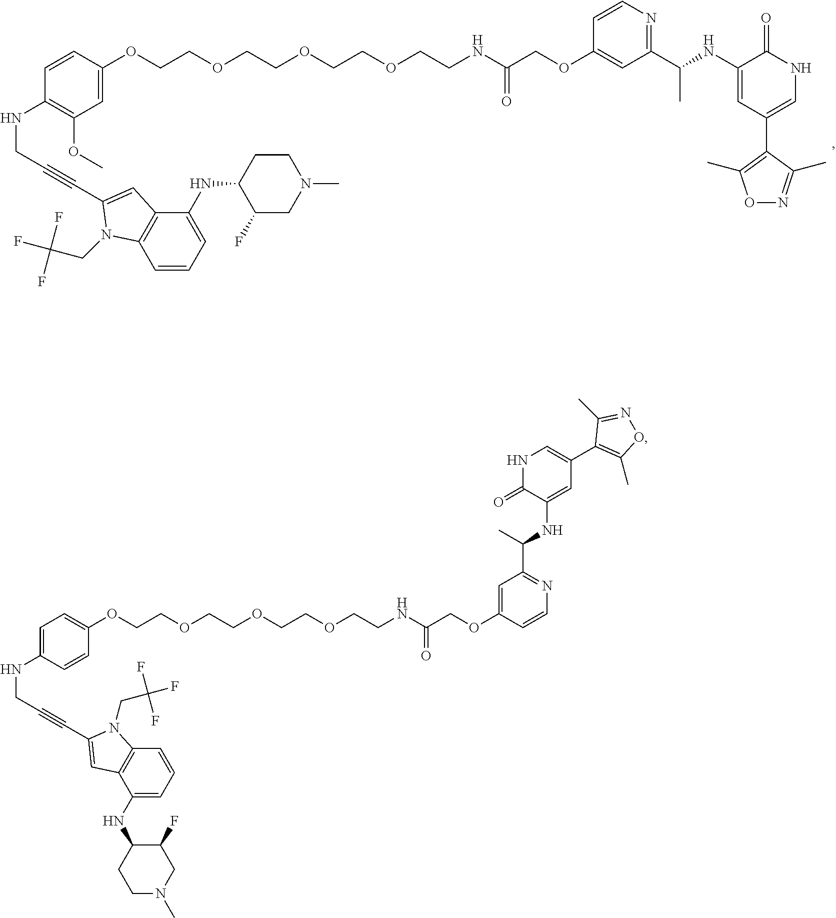 [SMILES: COc1cc(OCCOCCOCCOCCNC(=O)COc2ccnc([C@@H](C)Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; Cc1noc(C)c1-c1c[nH]c(=O)c(N[C@H](C)c2cc(OCC(=O)NCCOCCOCCOCCOc3ccc(NCC#Cc4cc5c(N[C@@H]6CCN(C)C[C@@H]6F)cccc5n4CC(F)(F)F)cc3)ccn2)c1]

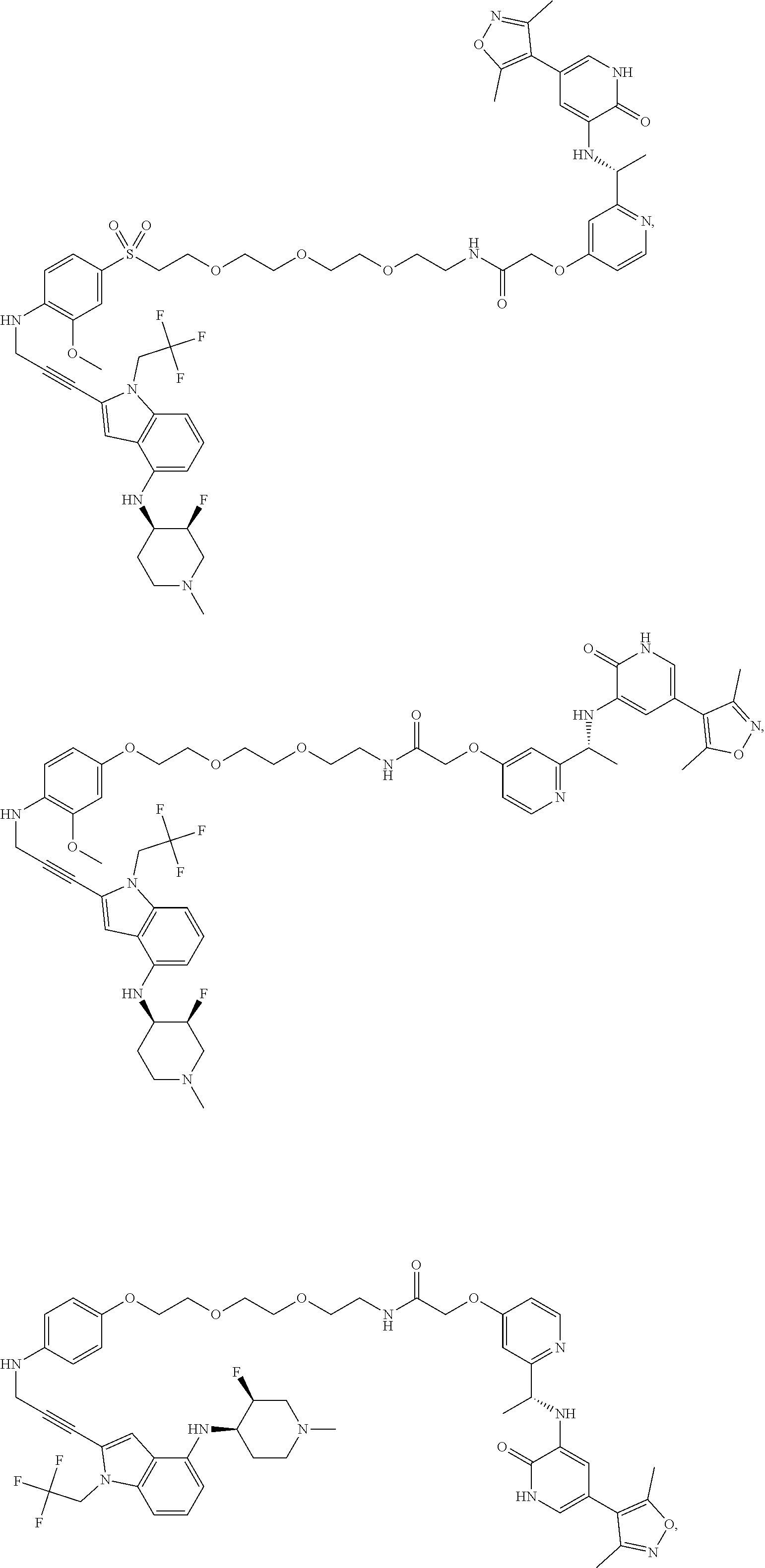 [SMILES: COc1cc(S(=O)(=O)CCOCCOCCOCCNC(=O)COc2ccnc([C@@H](C)Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; COc1cc(OCCOCCOCCNC(=O)COc2ccnc([C@@H](C)Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; Cc1noc(C)c1-c1c[nH]c(=O)c(N[C@H](C)c2cc(OCC(=O)NCCOCCOCCOc3ccc(NCC#Cc4cc5c(N[C@@H]6CCN(C)C[C@@H]6F)cccc5n4CC(F)(F)F)cc3)ccn2)c1]

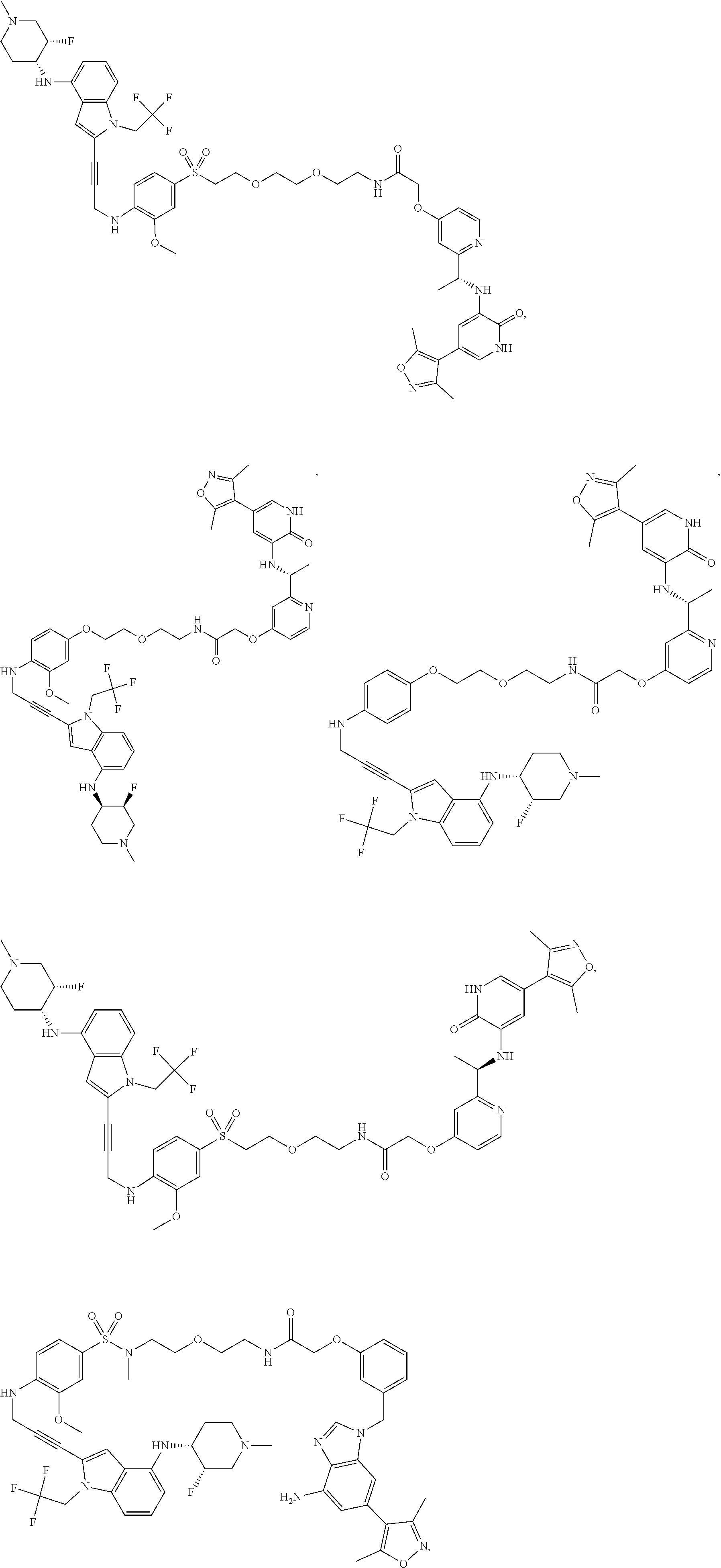

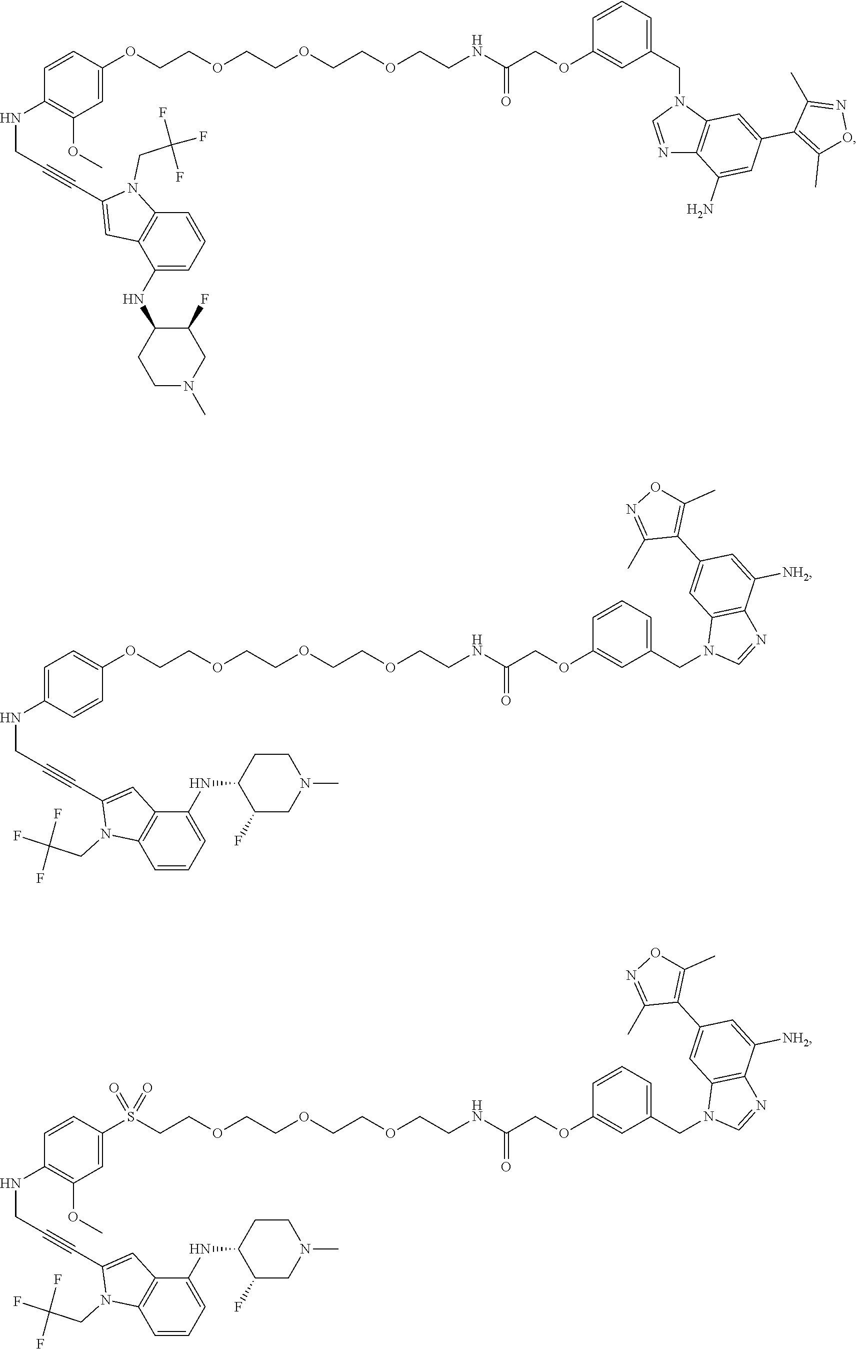 [SMILES: COc1cc(OCCOCCOCCOCCNC(=O)COc2cccc(Cn3cnc4c(N)cc(-c5c(C)noc5C)cc43)c2)ccc1NCC#Cc1cc2c(N[C@@H]3CCN(C)C[C@@H]3F)cccc2n1CC(F)(F)F ; Cc1noc(C)c1-c1cc(N)c2ncn(Cc3cccc(OCC(=O)NCCOCCOCCOCCOc4ccc(NCC#Cc5cc6c(N[C@@H]7CCN(C)C[C@@H]7F)cccc6n5CC(F)(F)F)cc4)c3)c2c1 ; COc1cc(S(=O)(=O)CCOCCOCCOCCNC(=O)COc2cccc(Cn3cnc4c(N)cc(-c5c(C)noc5C)cc43)c2)ccc1NCC#Cc1cc2c(N[C@@H]3CCN(C)C[C@@H]3F)cccc2n1CC(F)(F)F]

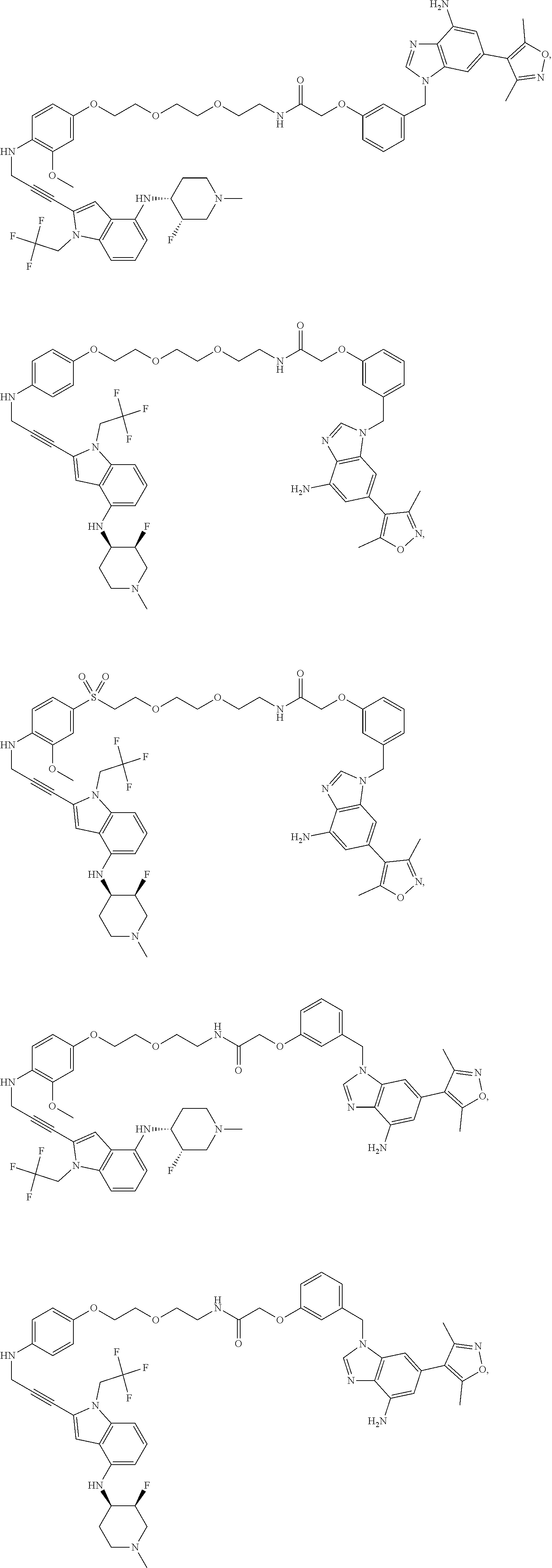

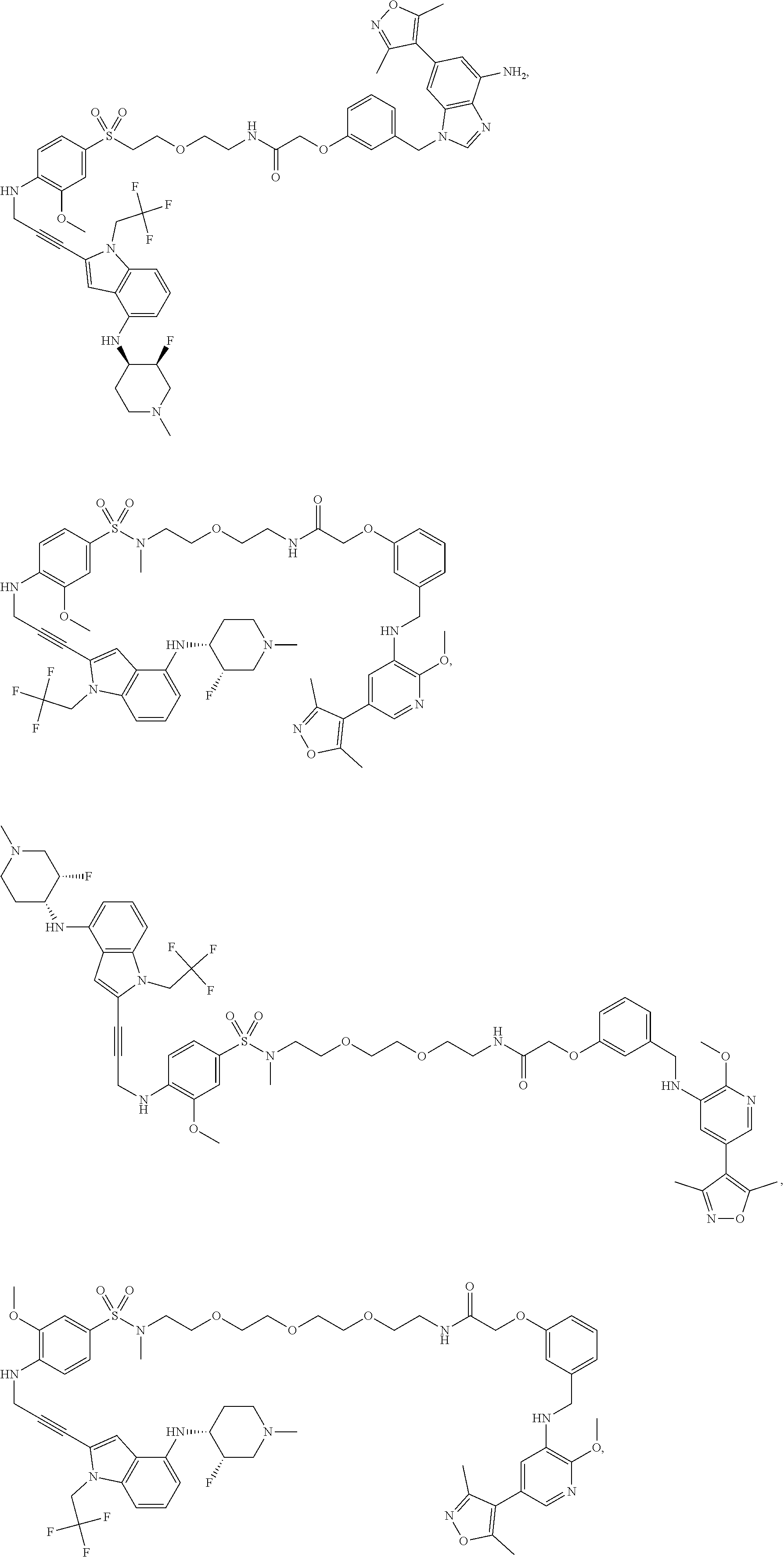

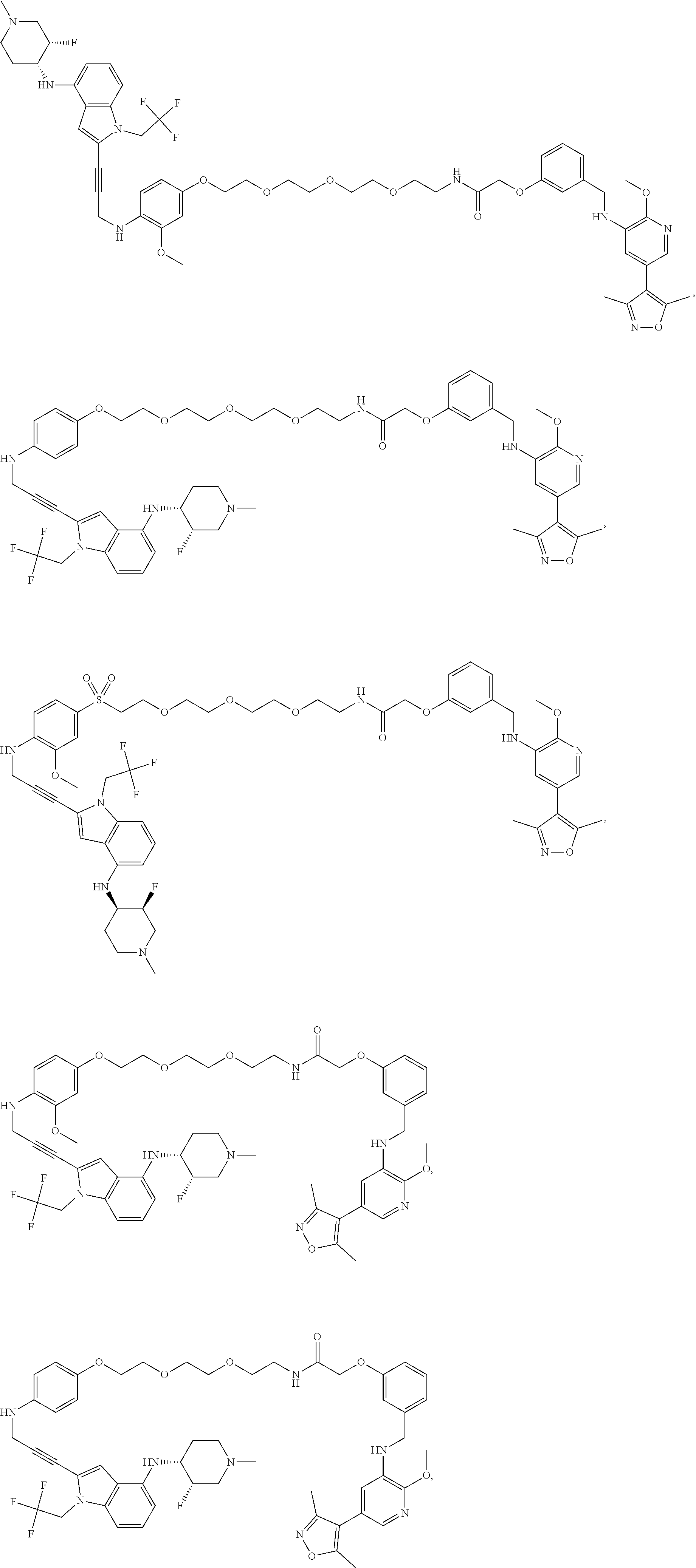

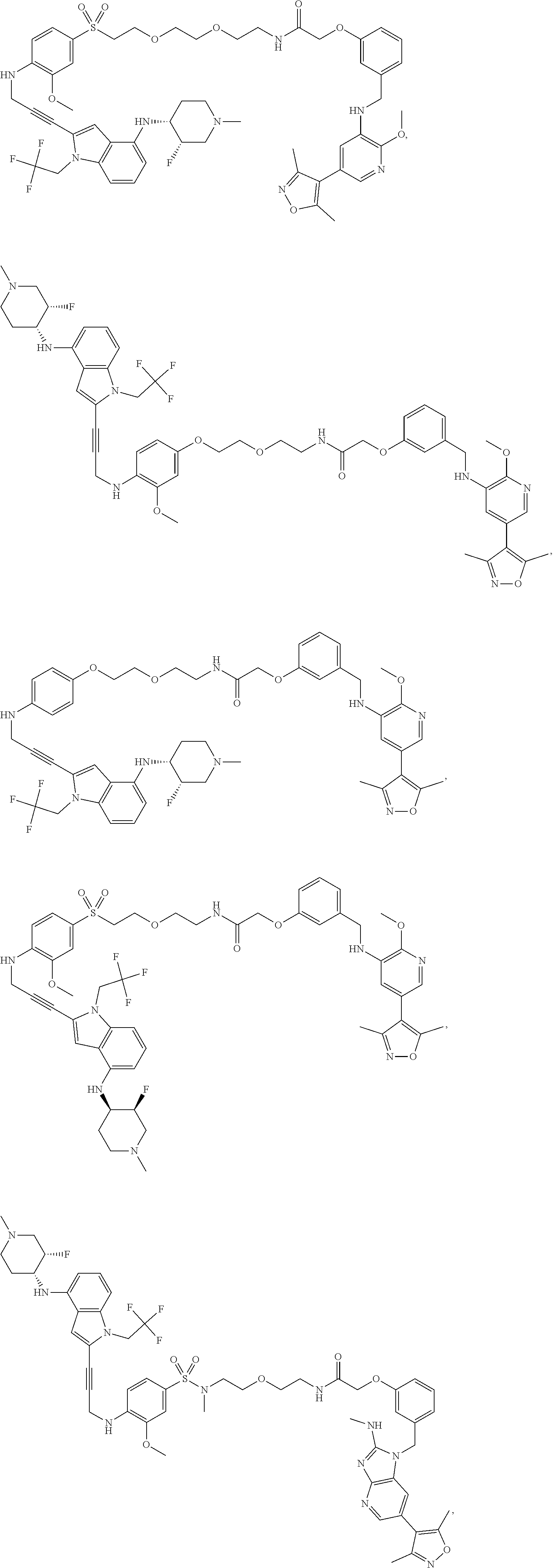

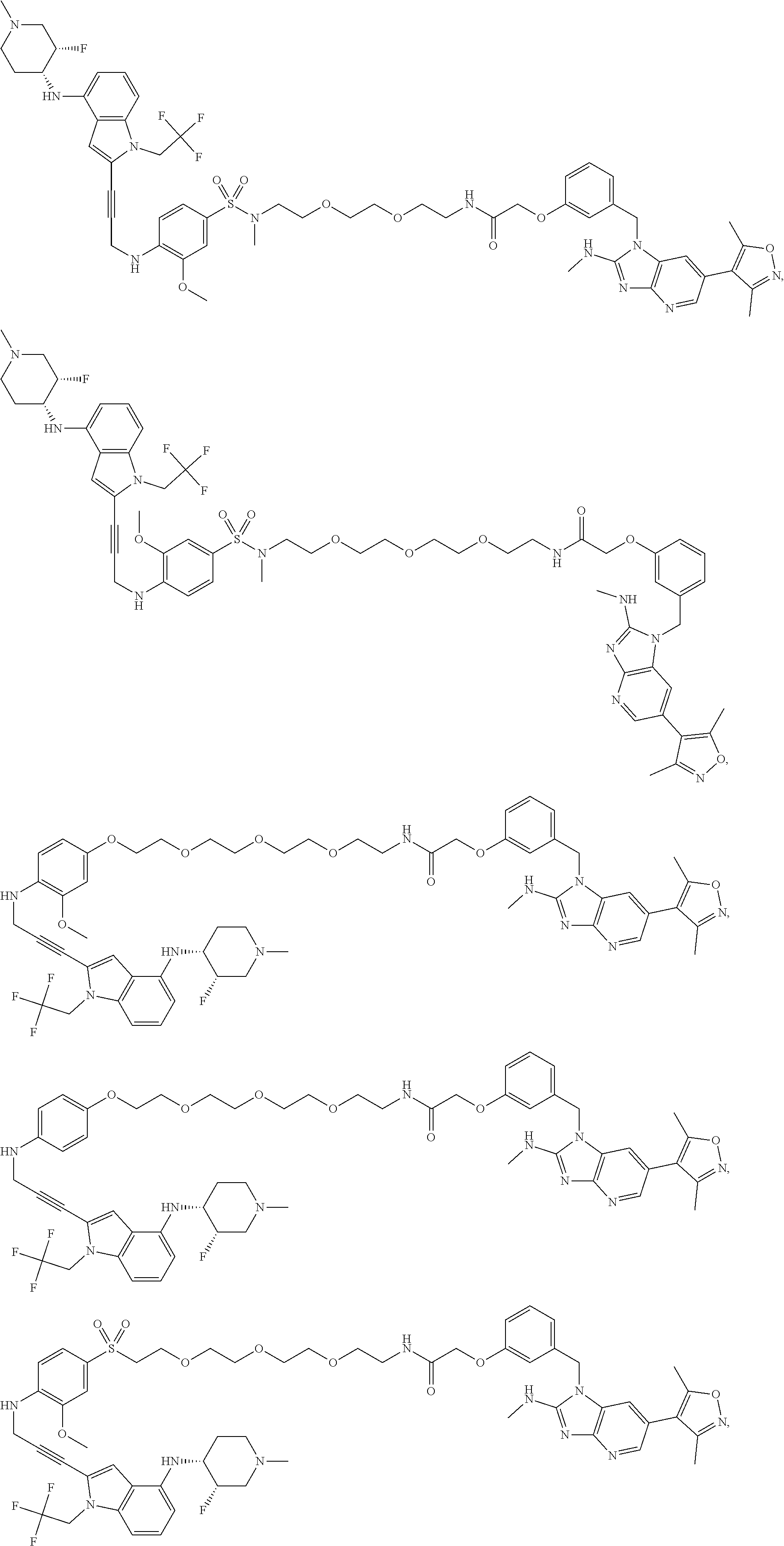

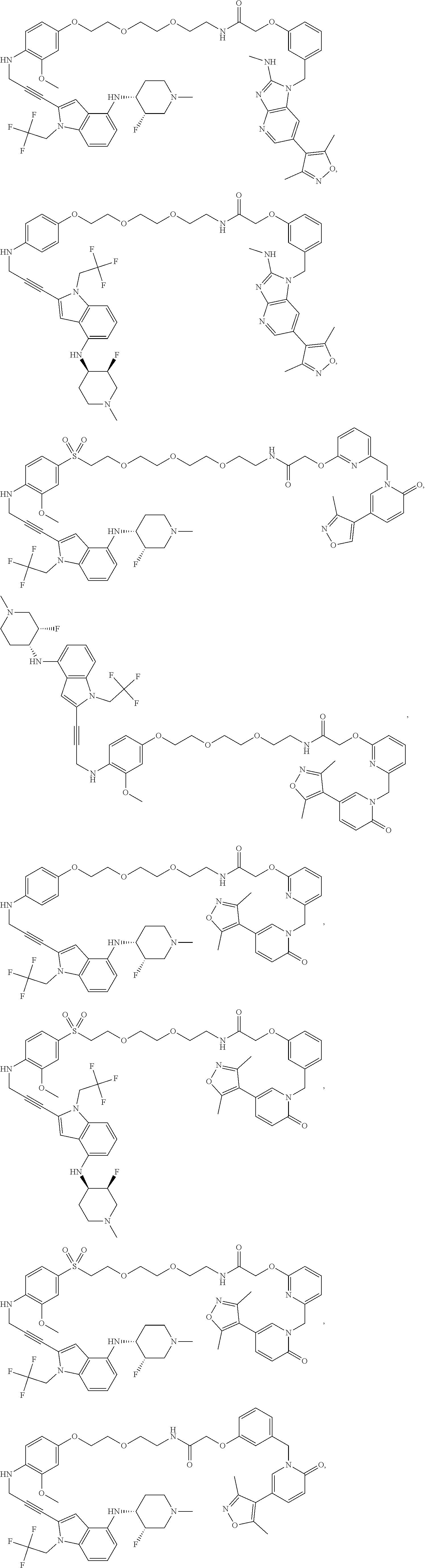

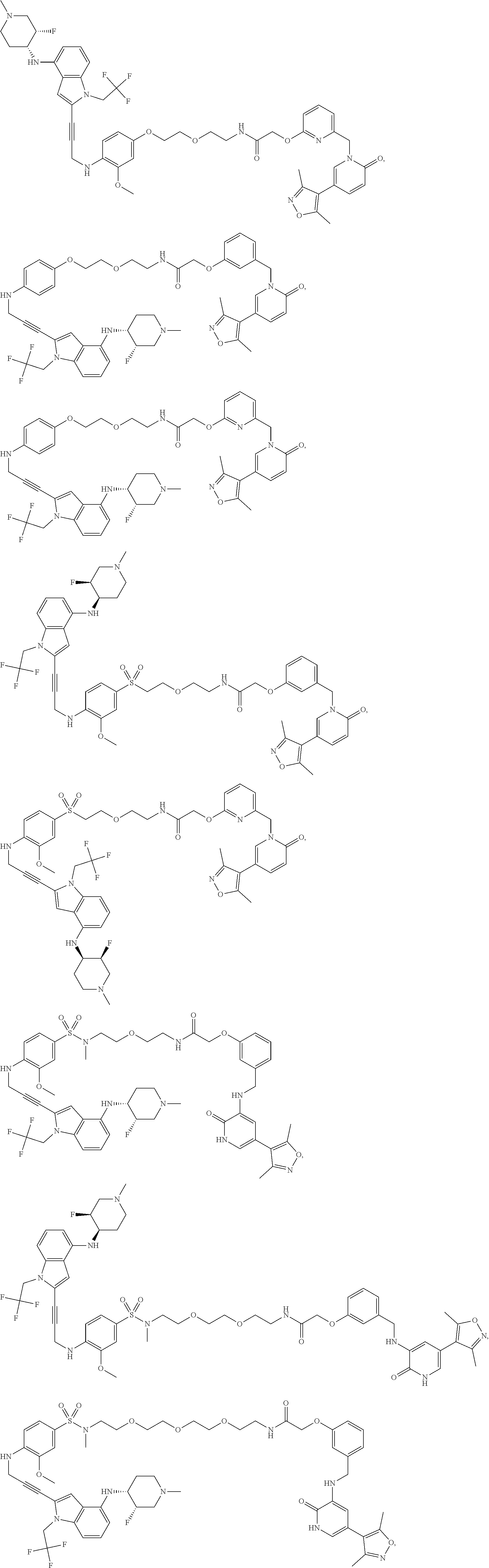

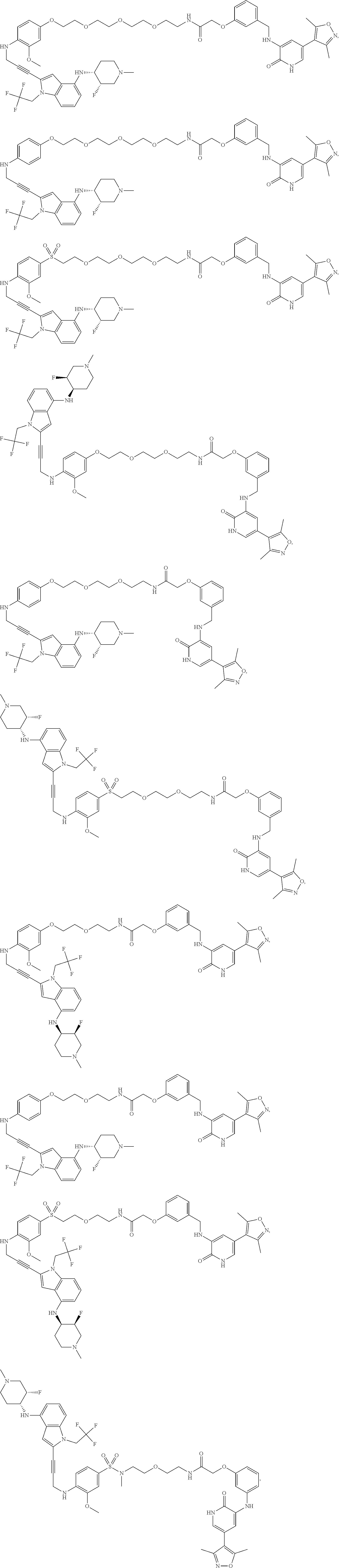

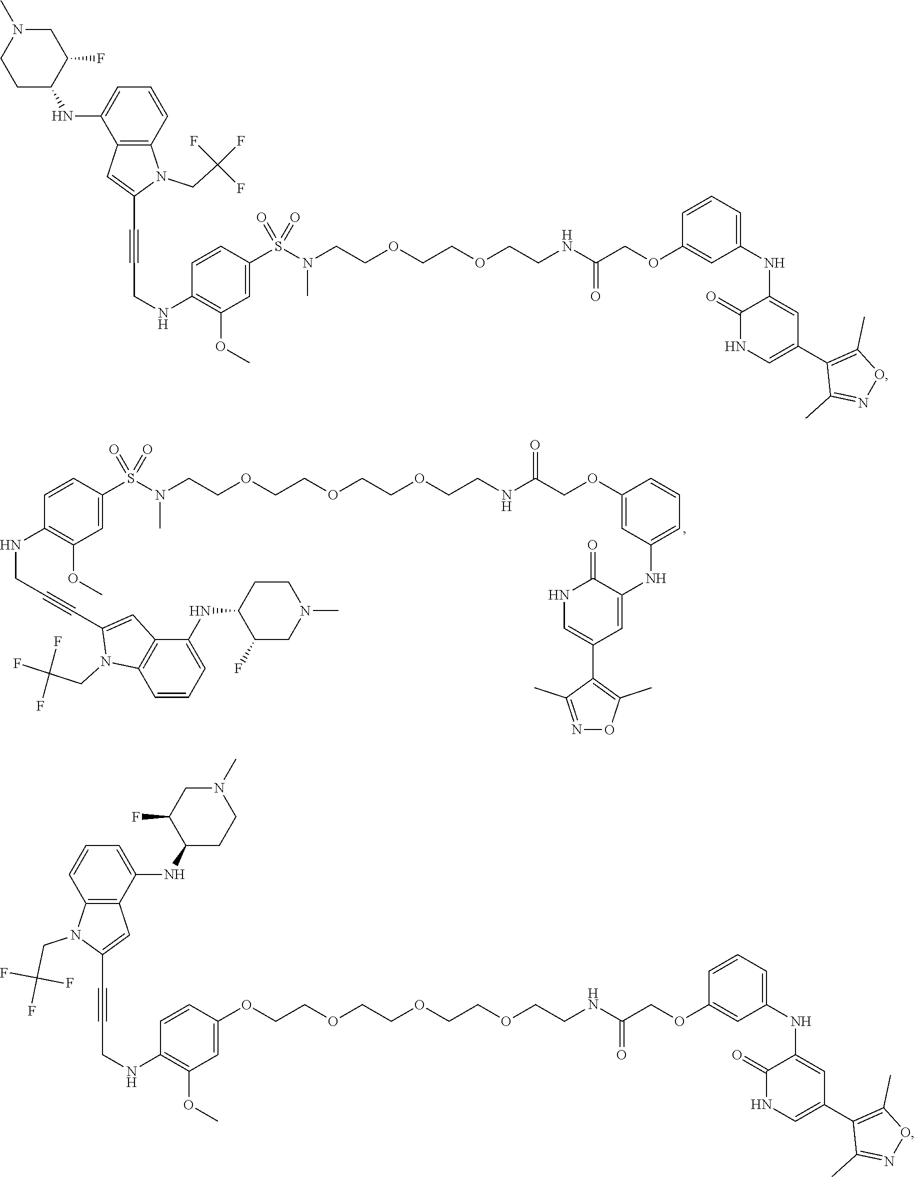 [SMILES: COc1cc(S(=O)(=O)N(C)CCOCCOCCNC(=O)COc2cccc(Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; COc1cc(S(=O)(=O)N(C)CCOCCOCCOCCNC(=O)COc2cccc(Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; COc1cc(OCCOCCOCCOCCNC(=O)COc2cccc(Nc3cc(-c4c(C)noc4C)c[nH]c3=O)c2)ccc1NCC#Cc1cc2c(N[C@@H]3CCN(C)C[C@@H]3F)cccc2n1CC(F)(F)F]

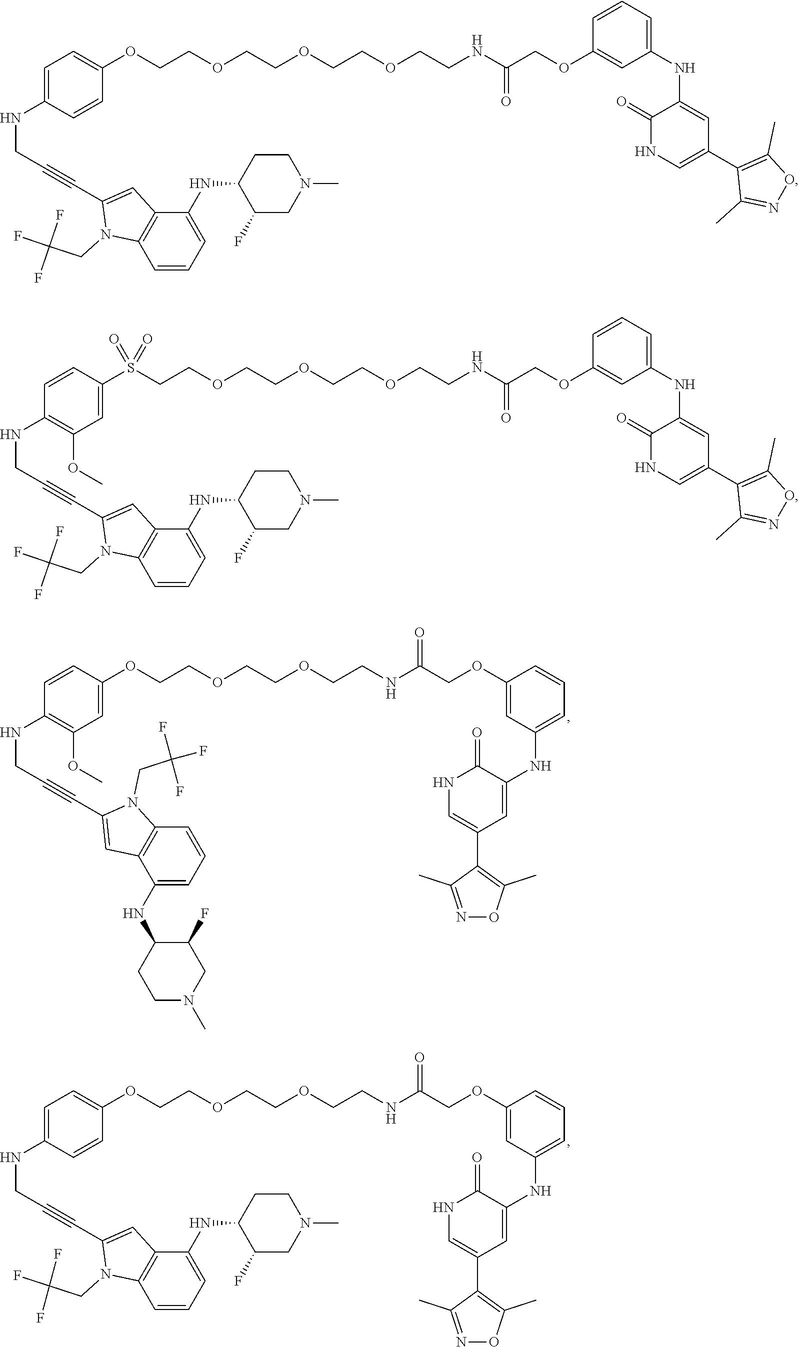 [SMILES: Cc1noc(C)c1-c1c[nH]c(=O)c(Nc2cccc(OCC(=O)NCCOCCOCCOCCOc3ccc(NCC#Cc4cc5c(N[C@@H]6CCN(C)C[C@@H]6F)cccc5n4CC(F)(F)F)cc3)c2)c1 ; COc1cc(S(=O)(=O)CCOCCOCCOCCNC(=O)COc2cccc(Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; COc1cc(OCCOCCOCCNC(=O)COc2cccc(Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; Cc1noc(C)c1-c1c[nH]c(=O)c(Nc2cccc(OCC(=O)NCCOCCOCCOc3ccc(NCC#Cc4cc5c(N[C@@H]6CCN(C)C[C@@H]6F)cccc5n4CC(F)(F)F)cc3)c2)c1]

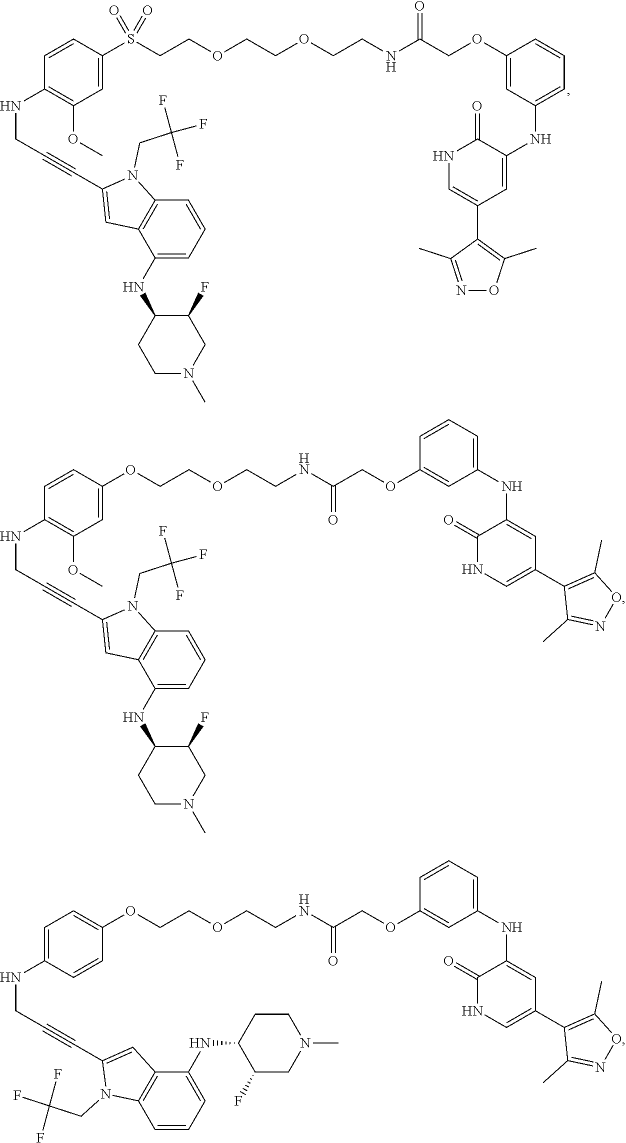 [SMILES: COc1cc(S(=O)(=O)CCOCCOCCNC(=O)COc2cccc(Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; COc1cc(OCCOCCNC(=O)COc2cccc(Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; Cc1noc(C)c1-c1c[nH]c(=O)c(Nc2cccc(OCC(=O)NCCOCCOc3ccc(NCC#Cc4cc5c(N[C@@H]6CCN(C)C[C@@H]6F)cccc5n4CC(F)(F)F)cc3)c2)c1]

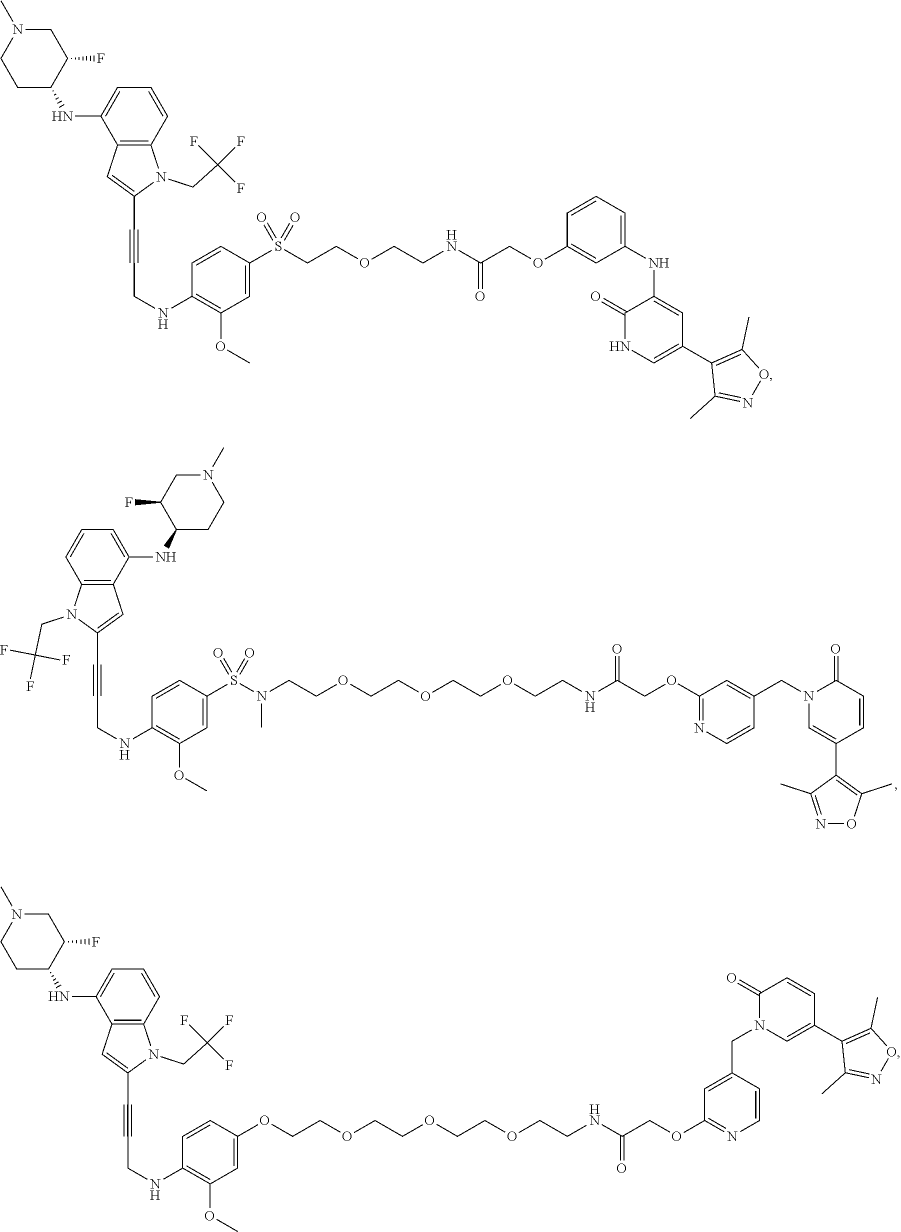 [SMILES: COc1cc(S(=O)(=O)CCOCCNC(=O)COc2cccc(Nc3cc(-c4c(C)noc4C)c[nH]c3=O)c2)ccc1NCC#Cc1cc2c(N[C@@H]3CCN(C)C[C@@H]3F)cccc2n1CC(F)(F)F ; COc1cc(S(=O)(=O)N(C)CCOCCOCCOCCNC(=O)COc2cc(Cn3cc(-c4c(C)noc4C)ccc3=O)ccn2)ccc1NCC#Cc1cc2c(N[C@@H]3CCN(C)C[C@@H]3F)cccc2n1CC(F)(F)F ; COc1cc(OCCOCCOCCOCCNC(=O)COc2cc(Cn3cc(-c4c(C)noc4C)ccc3=O)ccn2)ccc1NCC#Cc1cc2c(N[C@@H]3CCN(C)C[C@@H]3F)cccc2n1CC(F)(F)F]

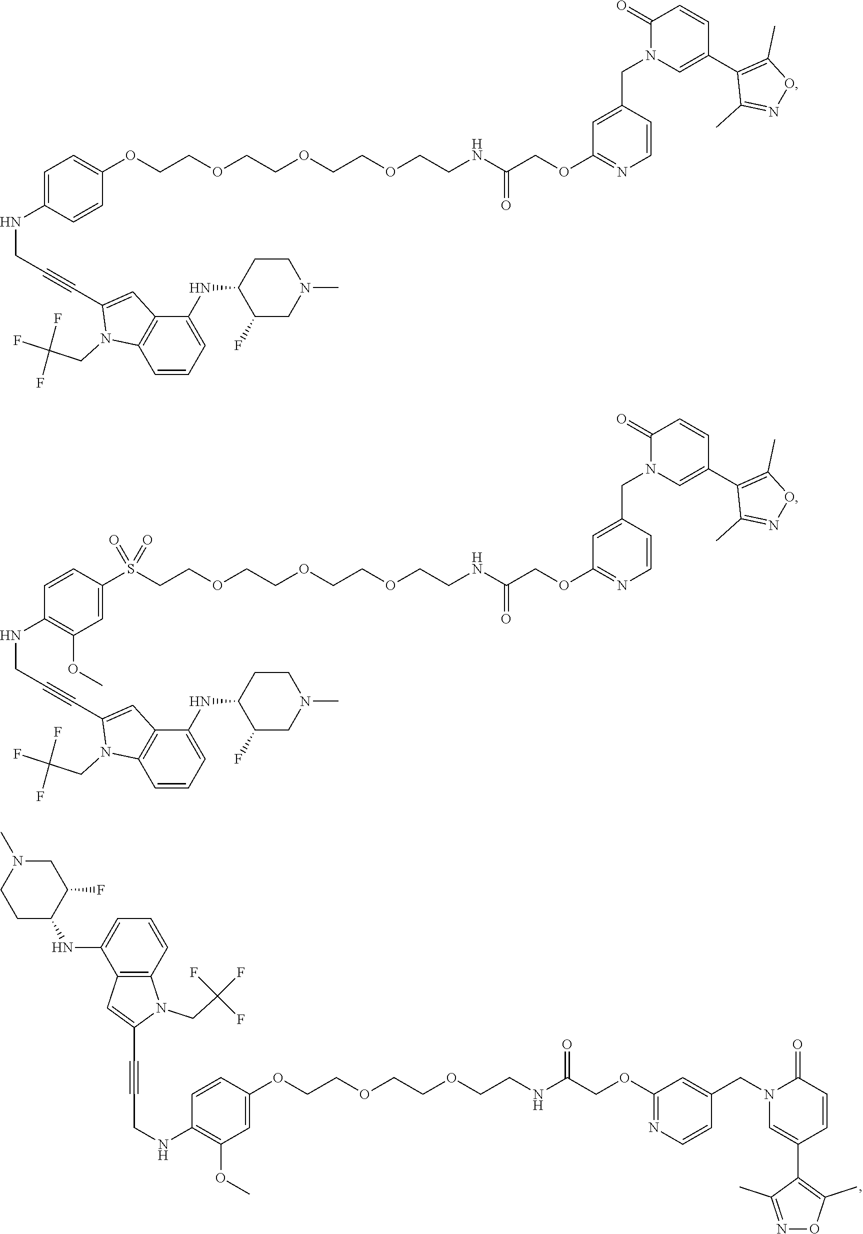 [SMILES: Cc1noc(C)c1-c1ccc(=O)n(Cc2ccnc(OCC(=O)NCCOCCOCCOCCOc3ccc(NCC#Cc4cc5c(N[C@@H]6CCN(C)C[C@@H]6F)cccc5n4CC(F)(F)F)cc3)c2)c1 ; COc1cc(S(=O)(=O)CCOCCOCCOCCNC(=O)COc2cc(Cn3cc(-c4c(C)noc4C)ccc3=O)ccn2)ccc1NCC#Cc1cc2c(N[C@@H]3CCN(C)C[C@@H]3F)cccc2n1CC(F)(F)F ; COc1cc(OCCOCCOCCNC(=O)COc2cc(Cn3cc(-c4c(C)noc4C)ccc3=O)ccn2)ccc1NCC#Cc1cc2c(N[C@@H]3CCN(C)C[C@@H]3F)cccc2n1CC(F)(F)F]

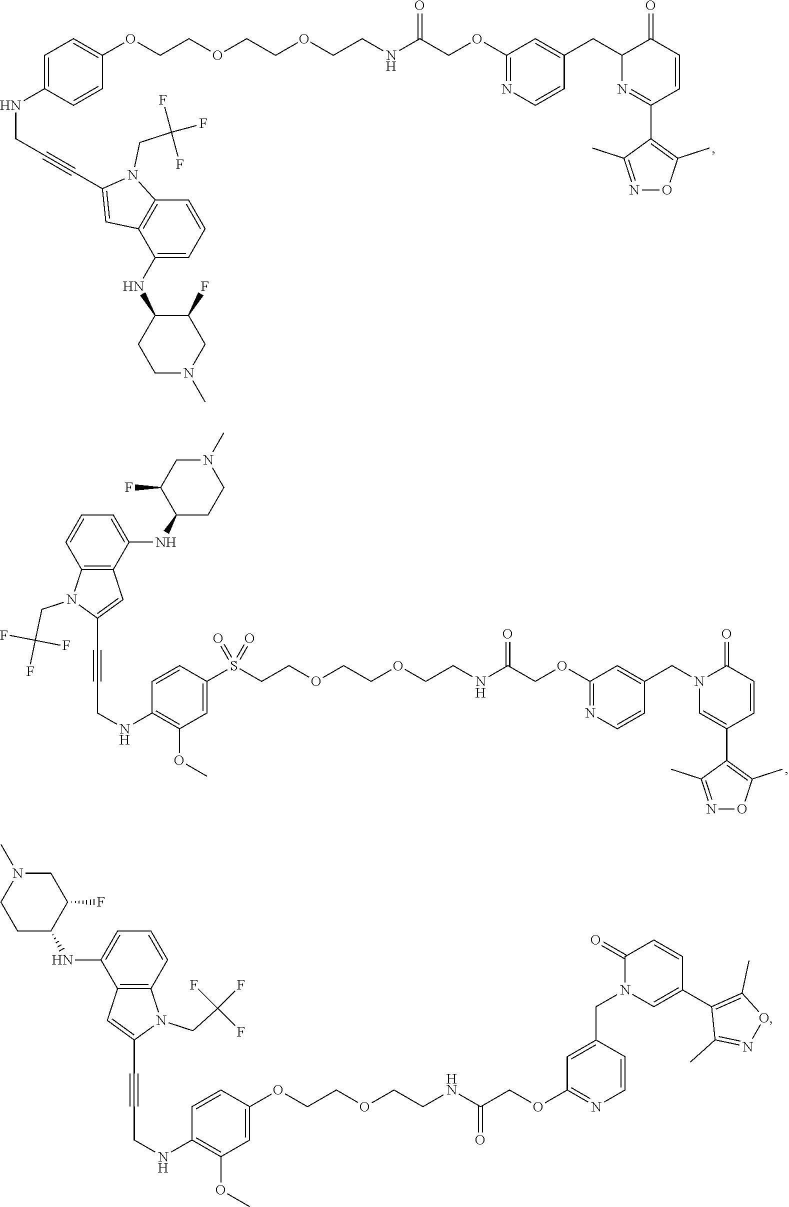

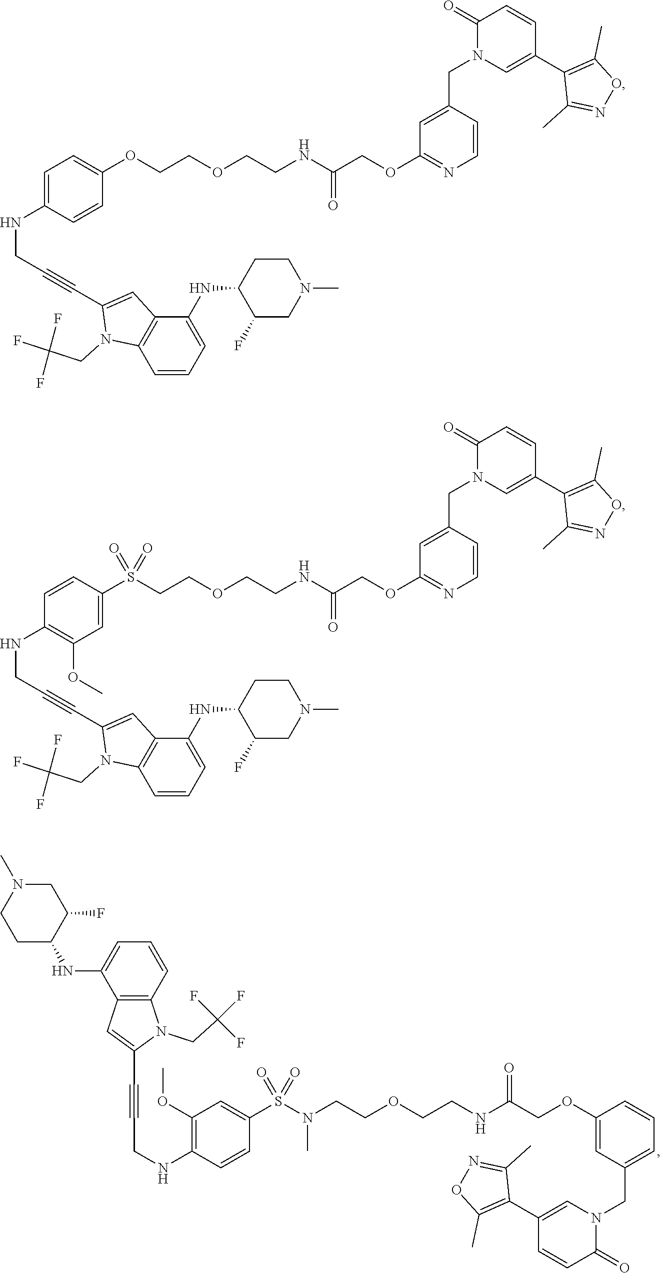 [SMILES: Cc1noc(C)c1-c1ccc(=O)n(Cc2ccnc(OCC(=O)NCCOCCOc3ccc(NCC#Cc4cc5c(N[C@@H]6CCN(C)C[C@@H]6F)cccc5n4CC(F)(F)F)cc3)c2)c1 ; COc1cc(S(=O)(=O)CCOCCNC(=O)COc2cc(Cn3cc(-c4c(C)noc4C)ccc3=O)ccn2)ccc1NCC#Cc1cc2c(N[C@@H]3CCN(C)C[C@@H]3F)cccc2n1CC(F)(F)F ; COc1cc(S(=O)(=O)N(C)CCOCCNC(=O)COc2cccc(Cn3cc(-c4c(C)noc4C)ccc3=O)c2)ccc1NCC#Cc1cc2c(N[C@@H]3CCN(C)C[C@@H]3F)cccc2n1CC(F)(F)F]

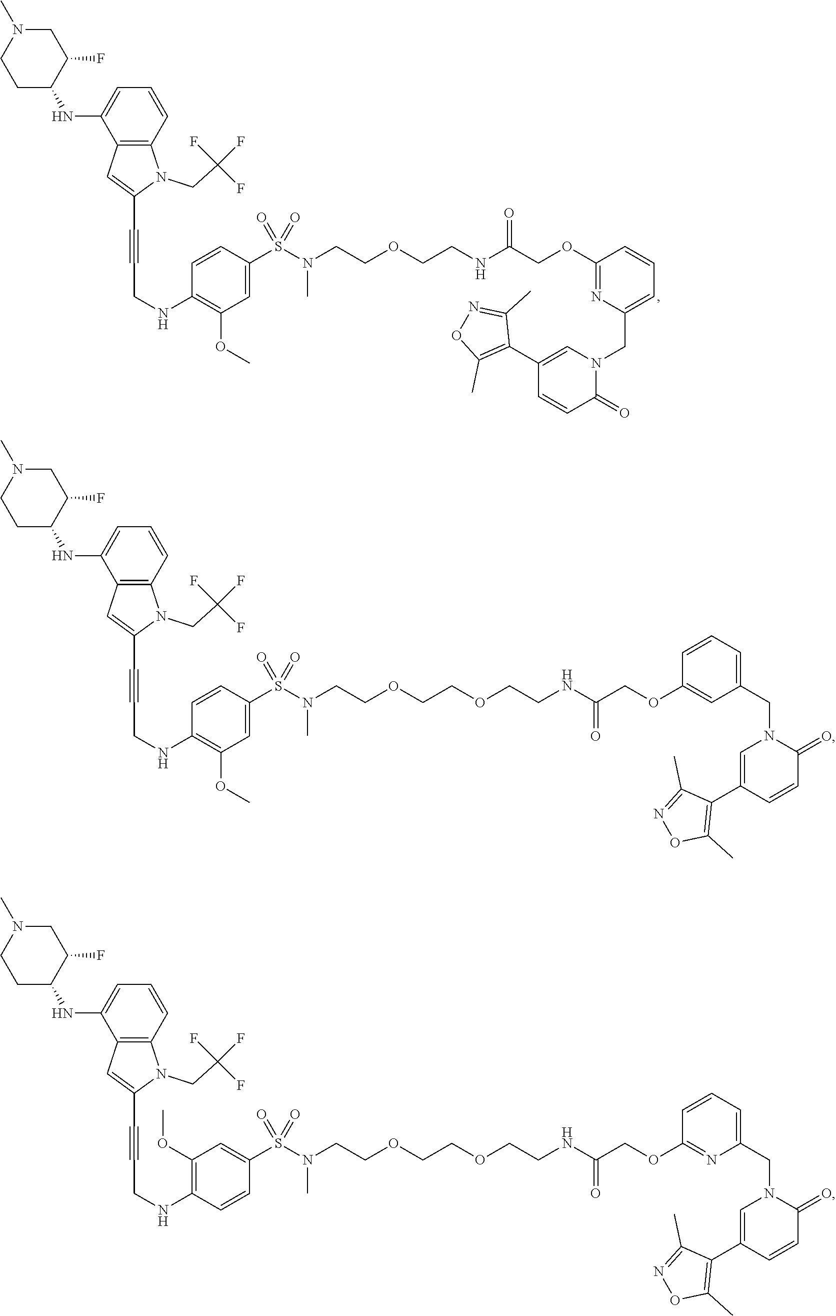 [SMILES: COc1cc(S(=O)(=O)N(C)CCOCCNC(=O)COc2cccc(Cn3cc(-c4c(C)noc4C)ccc3=O)n2)ccc1NCC#Cc1cc2c(N[C@@H]3CCN(C)C[C@@H]3F)cccc2n1CC(F)(F)F ; COc1cc(S(=O)(=O)N(C)CCOCCOCCNC(=O)COc2cccc(Cn3cc(-c4c(C)noc4C)ccc3=O)c2)ccc1NCC#Cc1cc2c(N[C@@H]3CCN(C)C[C@@H]3F)cccc2n1CC(F)(F)F ; COc1cc(S(=O)(=O)N(C)CCOCCOCCNC(=O)COc2cccc(Cn3cc(-c4c(C)noc4C)ccc3=O)n2)ccc1NCC#Cc1cc2c(N[C@@H]3CCN(C)C[C@@H]3F)cccc2n1CC(F)(F)F]

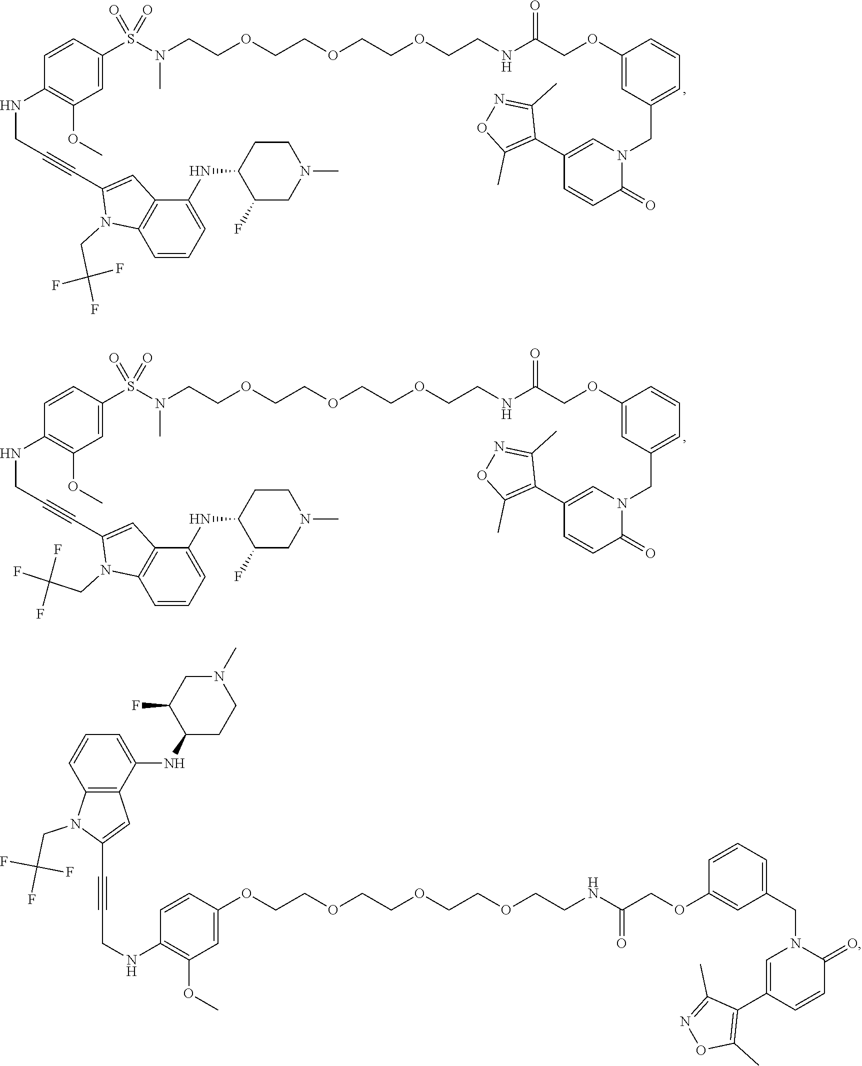 [SMILES: COc1cc(S(=O)(=O)N(C)CCOCCOCCOCCNC(=O)COc2cccc(Cn3cc(-c4c(C)noc4C)ccc3=O)c2)ccc1NCC#Cc1cc2c(N[C@@H]3CCN(C)C[C@@H]3F)cccc2n1CC(F)(F)F ; COc1cc(S(=O)(=O)N(C)CCOCCOCCOCCNC(=O)COc2cccc(Cn3cc(-c4c(C)noc4C)ccc3=O)c2)ccc1NCC#Cc1cc2c(N[C@@H]3CCN(C)C[C@@H]3F)cccc2n1CC(F)(F)F ; COc1cc(OCCOCCOCCOCCNC(=O)COc2cccc(Cn3cc(-c4c(C)noc4C)ccc3=O)c2)ccc1NCC#Cc1cc2c(N[C@@H]3CCN(C)C[C@@H]3F)cccc2n1CC(F)(F)F]

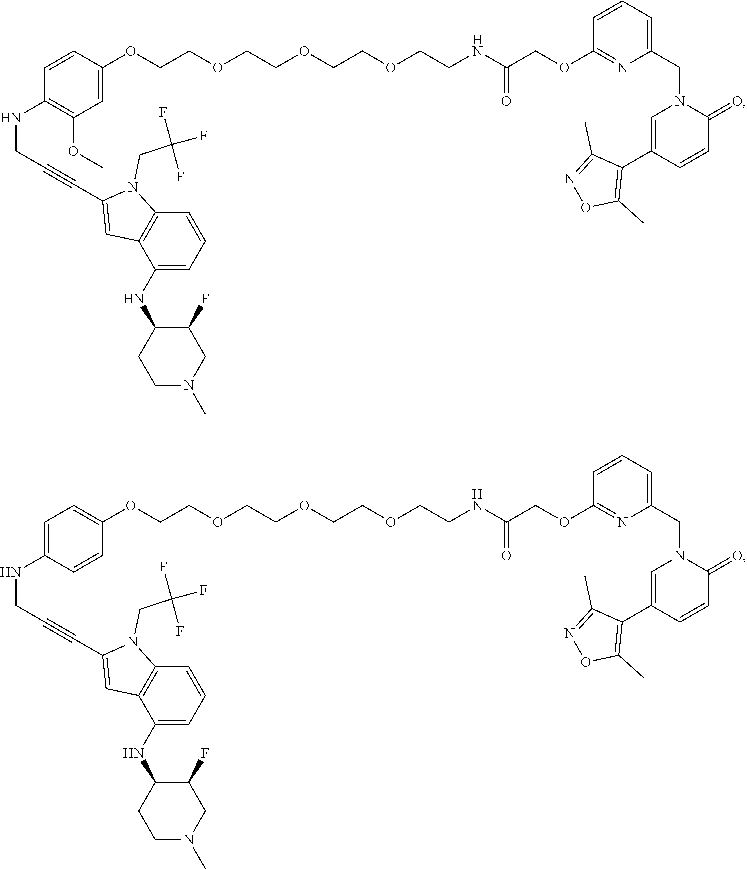 [SMILES: COc1cc(OCCOCCOCCOCCNC(=O)COc2cccc(Cn3cc(-c4c(C)noc4C)ccc3=O)n2)ccc1NCC#Cc1cc2c(N[C@@H]3CCN(C)C[C@@H]3F)cccc2n1CC(F)(F)F ; Cc1noc(C)c1-c1ccc(=O)n(Cc2cccc(OCC(=O)NCCOCCOCCOCCOc3ccc(NCC#Cc4cc5c(N[C@@H]6CCN(C)C[C@@H]6F)cccc5n4CC(F)(F)F)cc3)n2)c1]

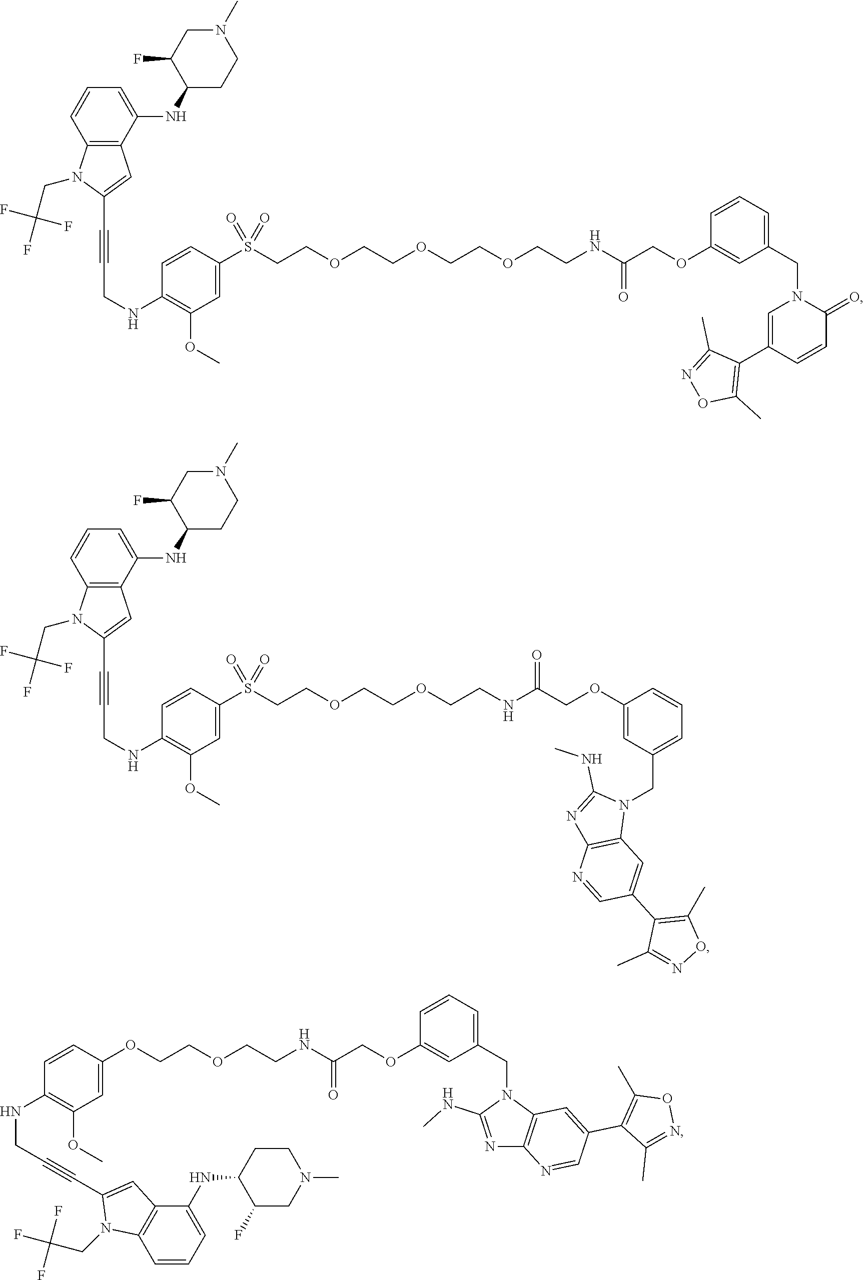 [SMILES: COc1cc(S(=O)(=O)CCOCCOCCOCCNC(=O)COc2cccc(Cn3cc(-c4c(C)noc4C)ccc3=O)c2)ccc1NCC#Cc1cc2c(N[C@@H]3CCN(C)C[C@@H]3F)cccc2n1CC(F)(F)F ; CNc1nc2ncc(-c3c(C)noc3C)cc2n1Cc1cccc(OCC(=O)NCCOCCOCCS(=O)(=O)c2ccc(NCC#Cc3cc4c(N[C@@H]5CCN(C)C[C@@H]5F)cccc4n3CC(F)(F)F)c(OC)c2)c1 ; CNc1nc2ncc(-c3c(C)noc3C)cc2n1Cc1cccc(OCC(=O)NCCOCCOc2ccc(NCC#Cc3cc4c(N[C@@H]5CCN(C)C[C@@H]5F)cccc4n3CC(F)(F)F)c(OC)c2)c1]

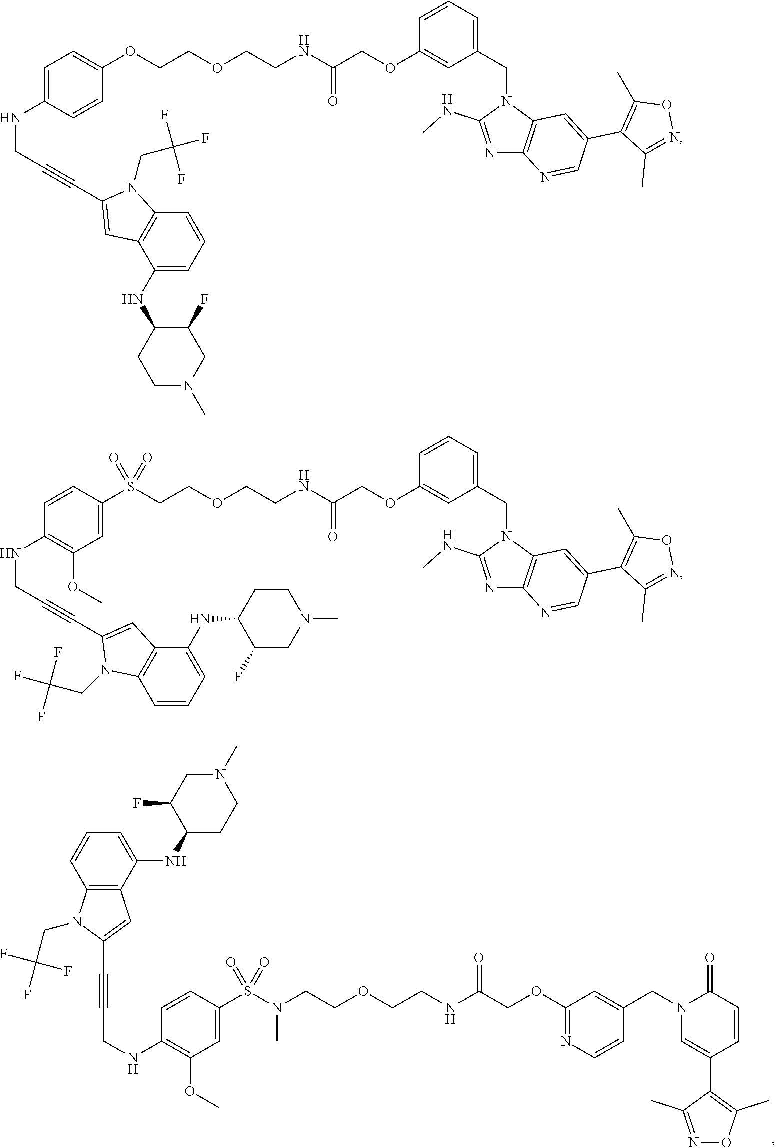 [SMILES: CNc1nc2ncc(-c3c(C)noc3C)cc2n1Cc1cccc(OCC(=O)NCCOCCOc2ccc(NCC#Cc3cc4c(N[C@@H]5CCN(C)C[C@@H]5F)cccc4n3CC(F)(F)F)cc2)c1 ; CNc1nc2ncc(-c3c(C)noc3C)cc2n1Cc1cccc(OCC(=O)NCCOCCS(=O)(=O)c2ccc(NCC#Cc3cc4c(N[C@@H]5CCN(C)C[C@@H]5F)cccc4n3CC(F)(F)F)c(OC)c2)c1 ; COc1cc(S(=O)(=O)N(C)CCOCCNC(=O)COc2cc(Cn3cc(-c4c(C)noc4C)ccc3=O)ccn2)ccc1NCC#Cc1cc2c(N[C@@H]3CCN(C)C[C@@H]3F)cccc2n1CC(F)(F)F]

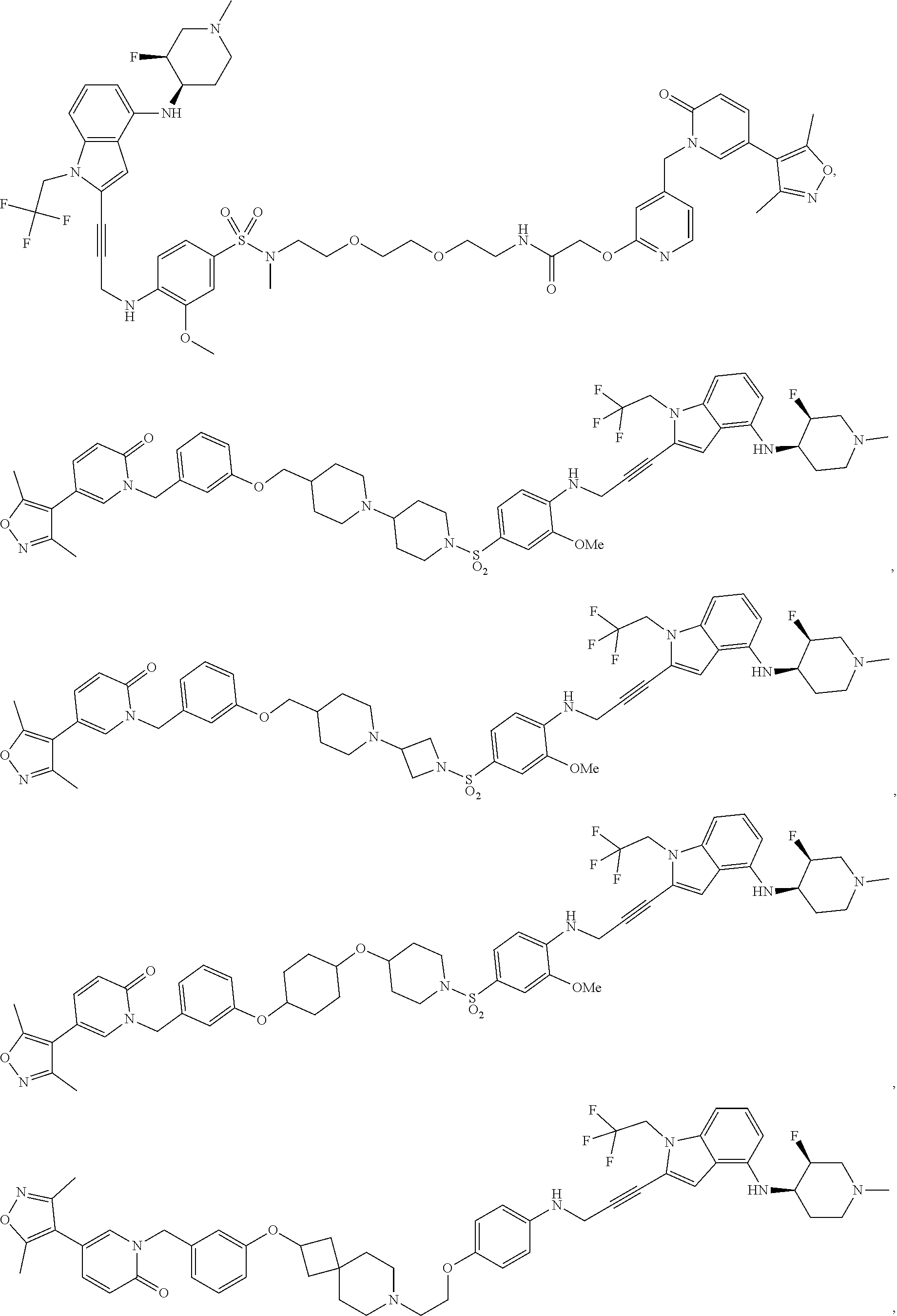

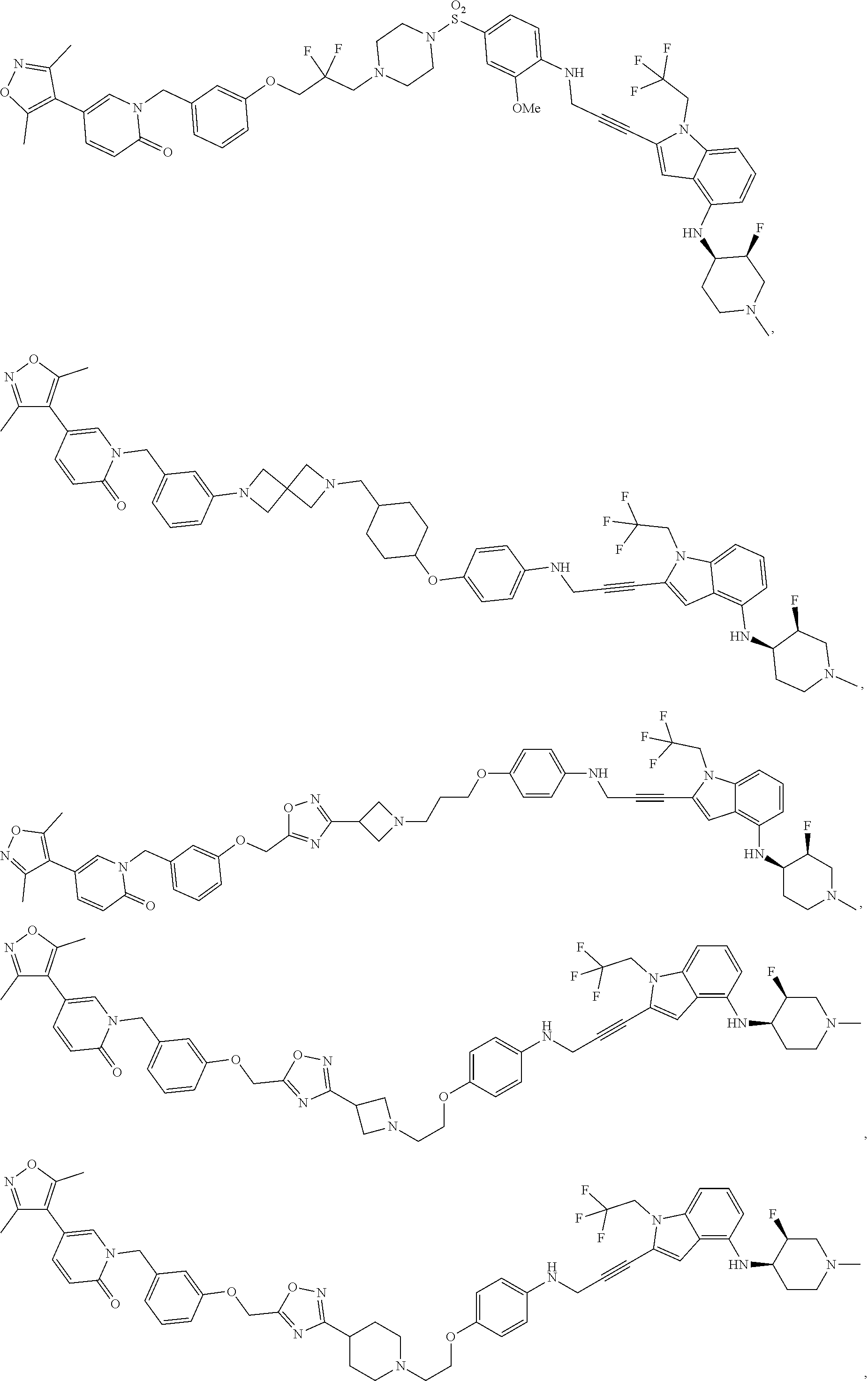

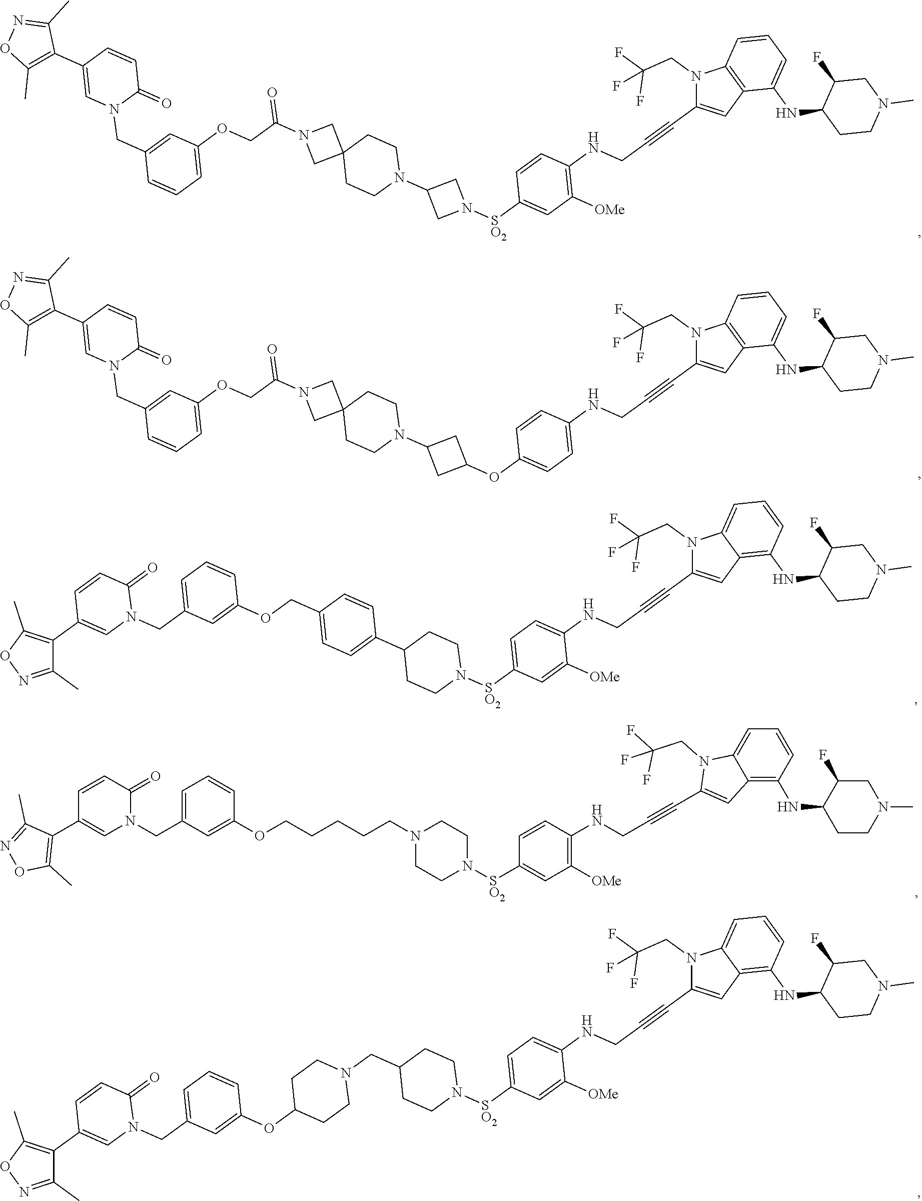

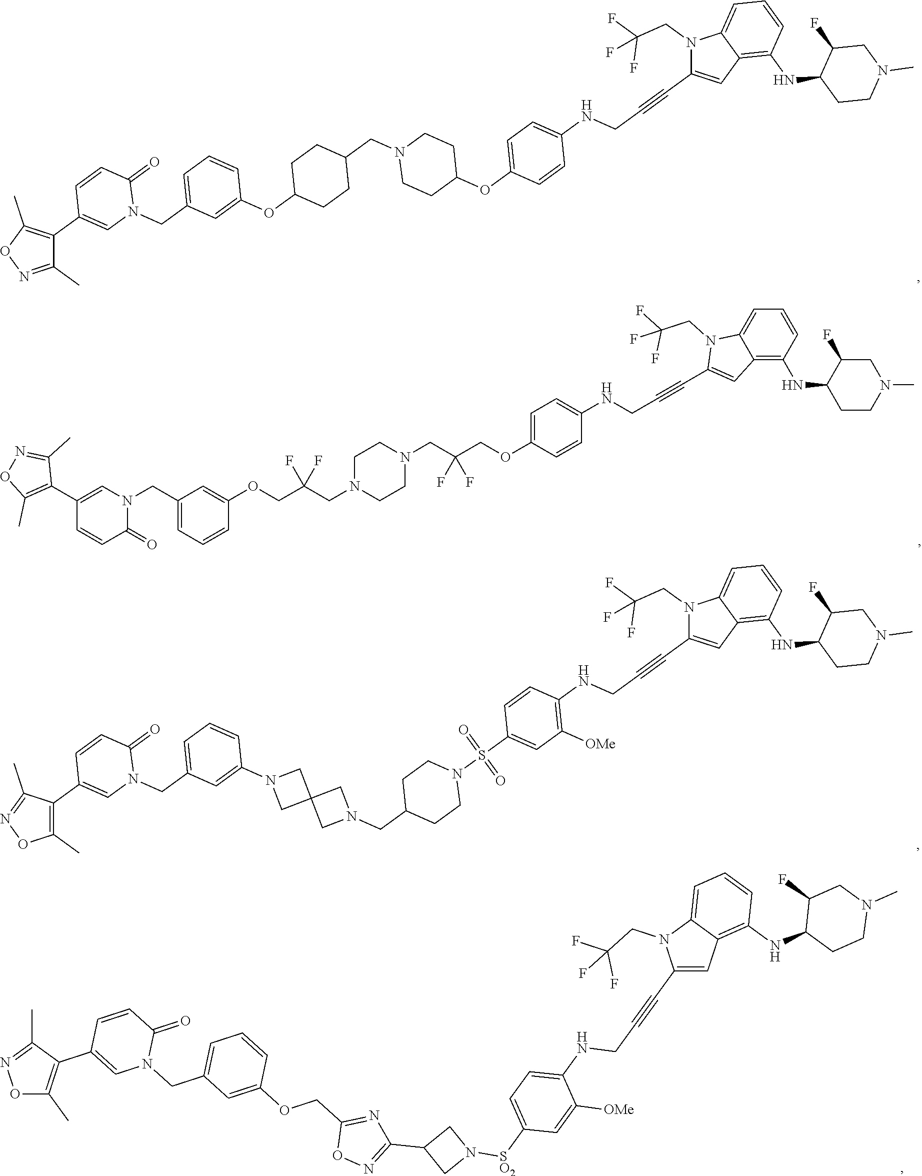

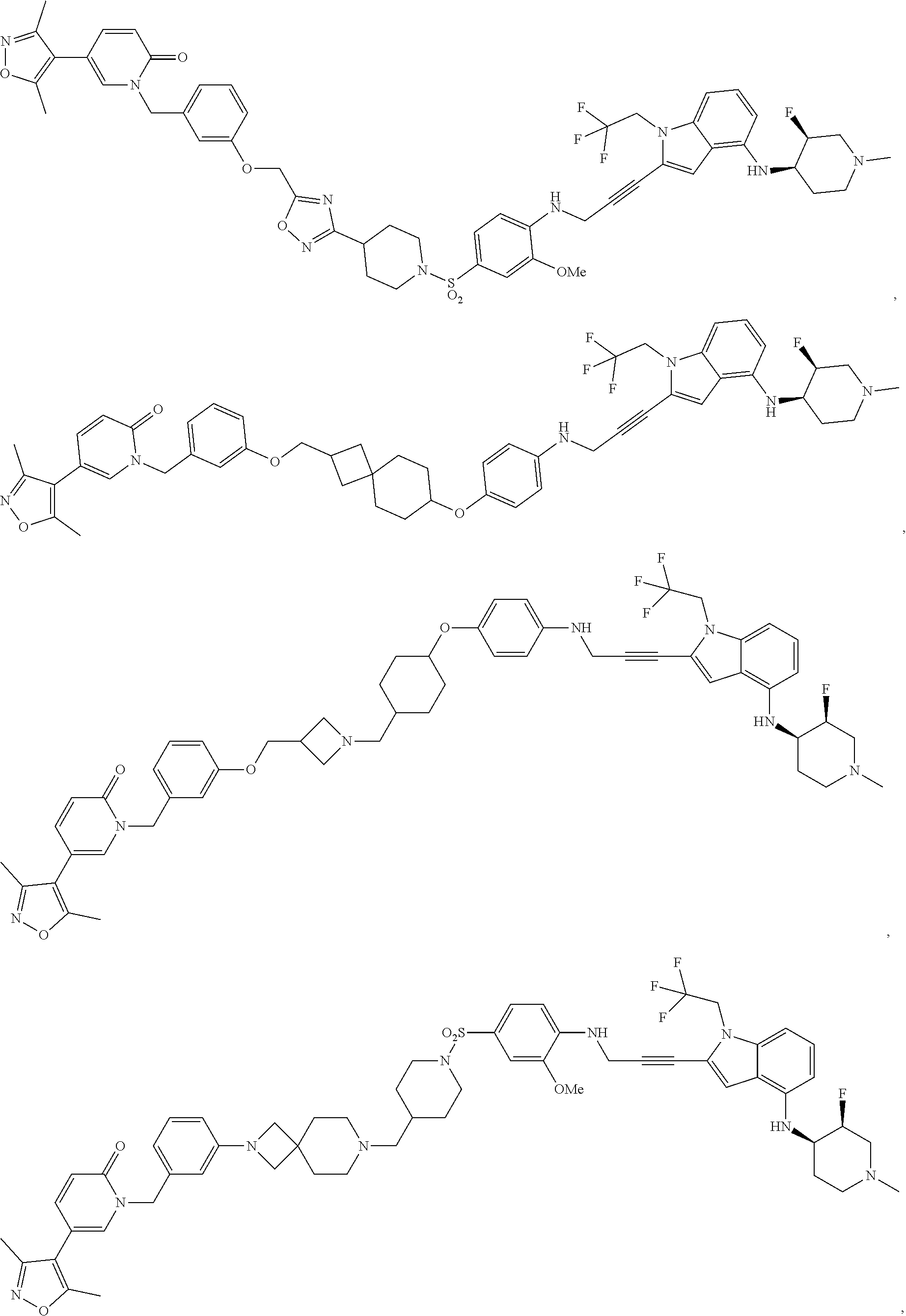

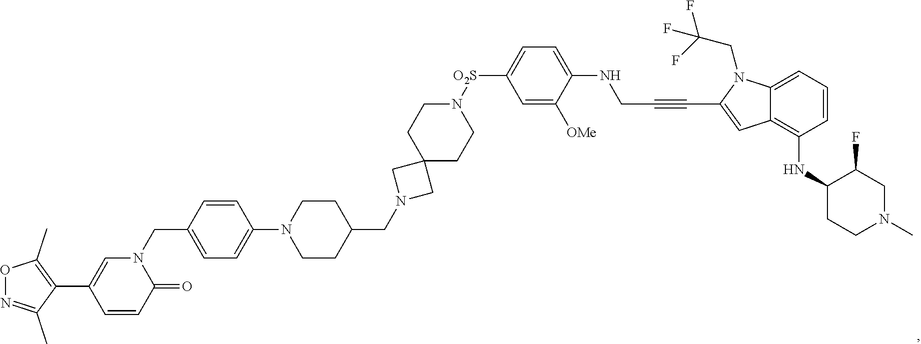 [SMILES: COc1cc(S(=O)(=O)N2CCC3(CC2)CN(CC2CCN(c4ccc(Cn5cc(-c6c(C)noc6C)ccc5=O)cc4)CC2)C3)ccc1NCC#Cc1cc2c(N[C@@H]3CCN(C)C[C@@H]3F)cccc2n1CC(F)(F)F]

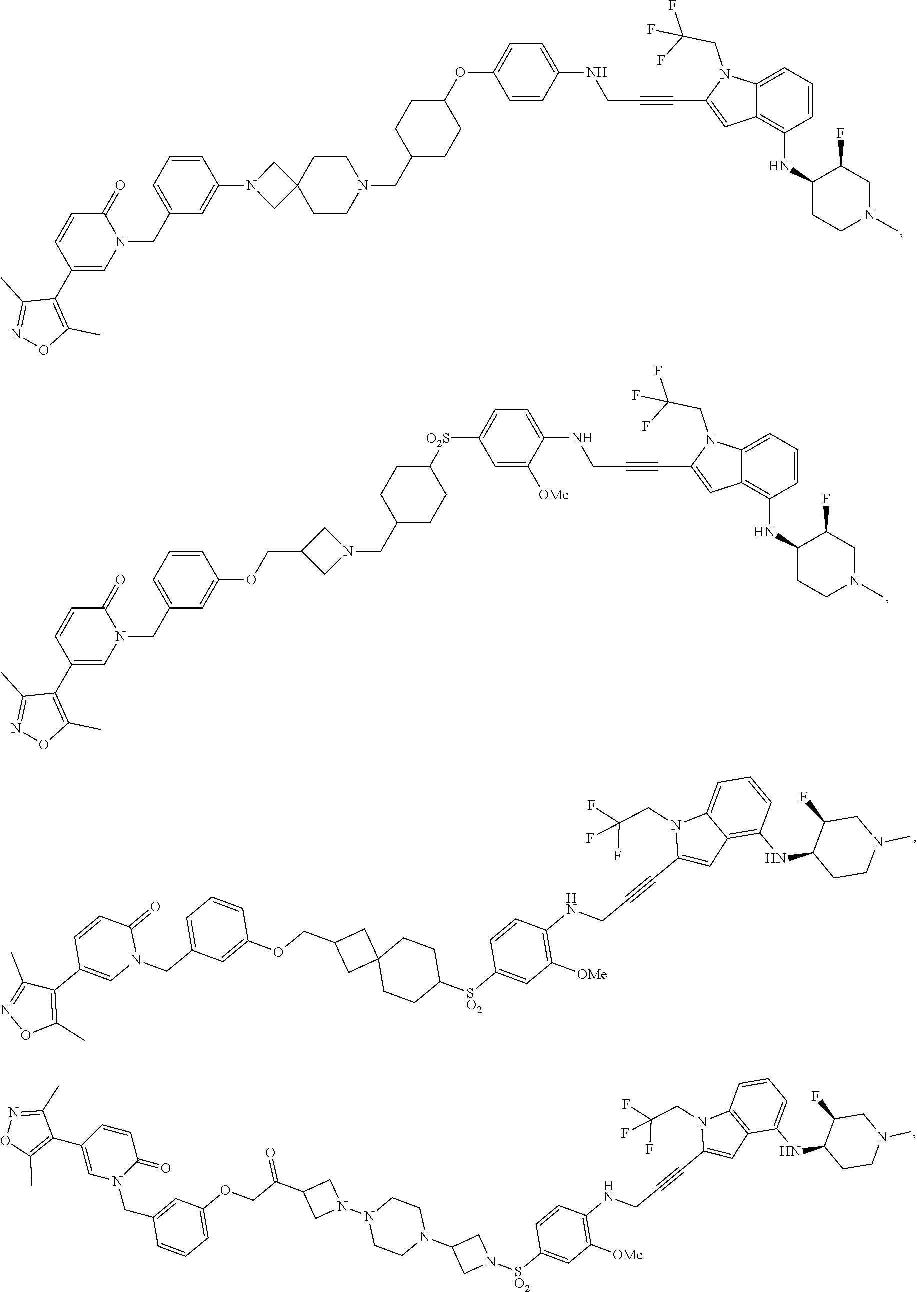

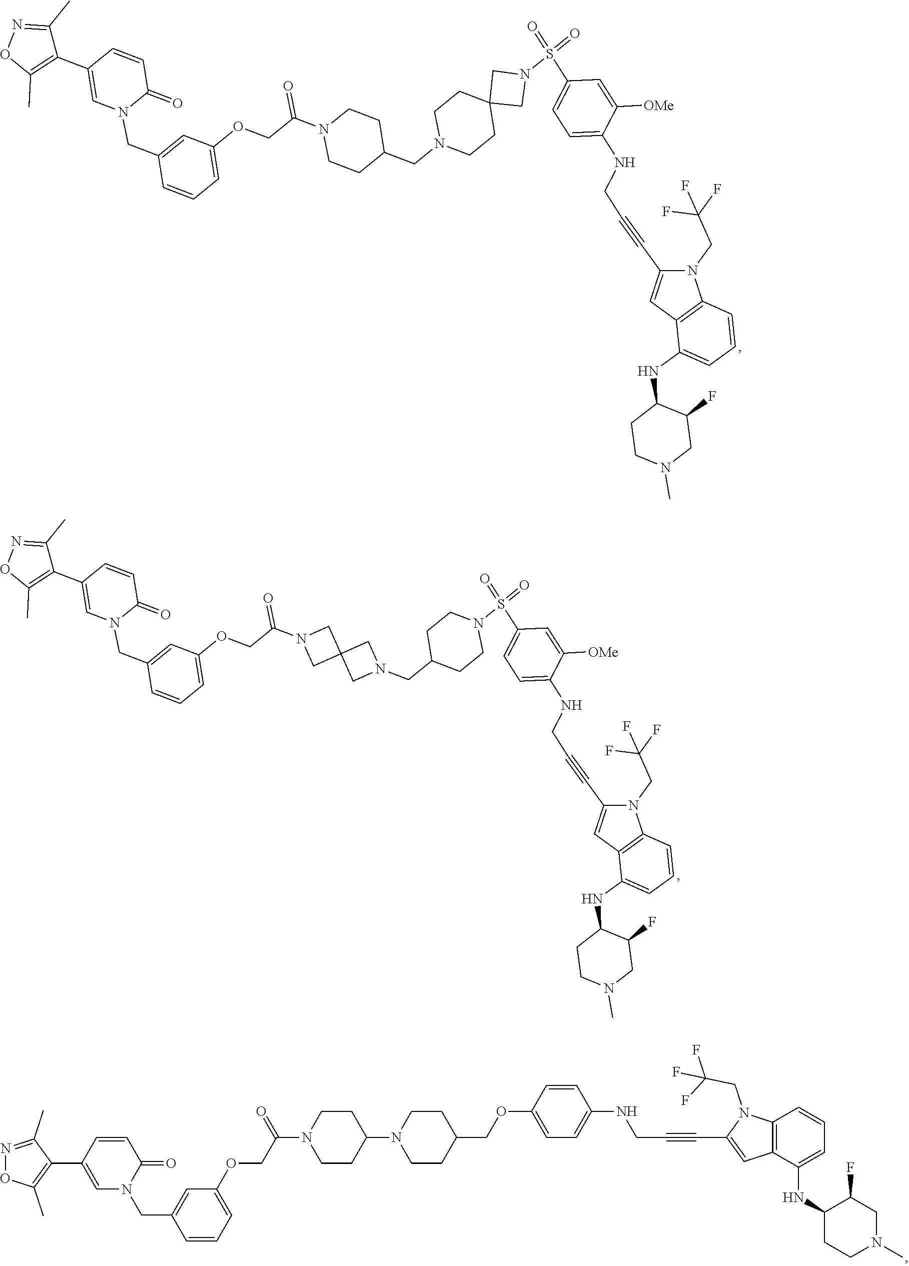 [SMILES: COc1cc(S(=O)(=O)N2CC3(CCN(CC4CCN(C(=O)COc5cccc(Cn6cc(-c7c(C)noc7C)ccc6=O)c5)CC4)CC3)C2)ccc1NCC#Cc1cc2c(N[C@@H]3CCN(C)C[C@@H]3F)cccc2n1CC(F)(F)F ; COc1cc(S(=O)(=O)N2CCC(CN3CC4(C3)CN(C(=O)COc3cccc(Cn5cc(-c6c(C)noc6C)ccc5=O)c3)C4)CC2)ccc1NCC#Cc1cc2c(N[C@@H]3CCN(C)C[C@@H]3F)cccc2n1CC(F)(F)F ; Cc1noc(C)c1-c1ccc(=O)n(Cc2cccc(OCC(=O)N3CCC(N4CCC(COc5ccc(NCC#Cc6cc7c(N[C@@H]8CCN(C)C[C@@H]8F)cccc7n6CC(F)(F)F)cc5)CC4)CC3)c2)c1]

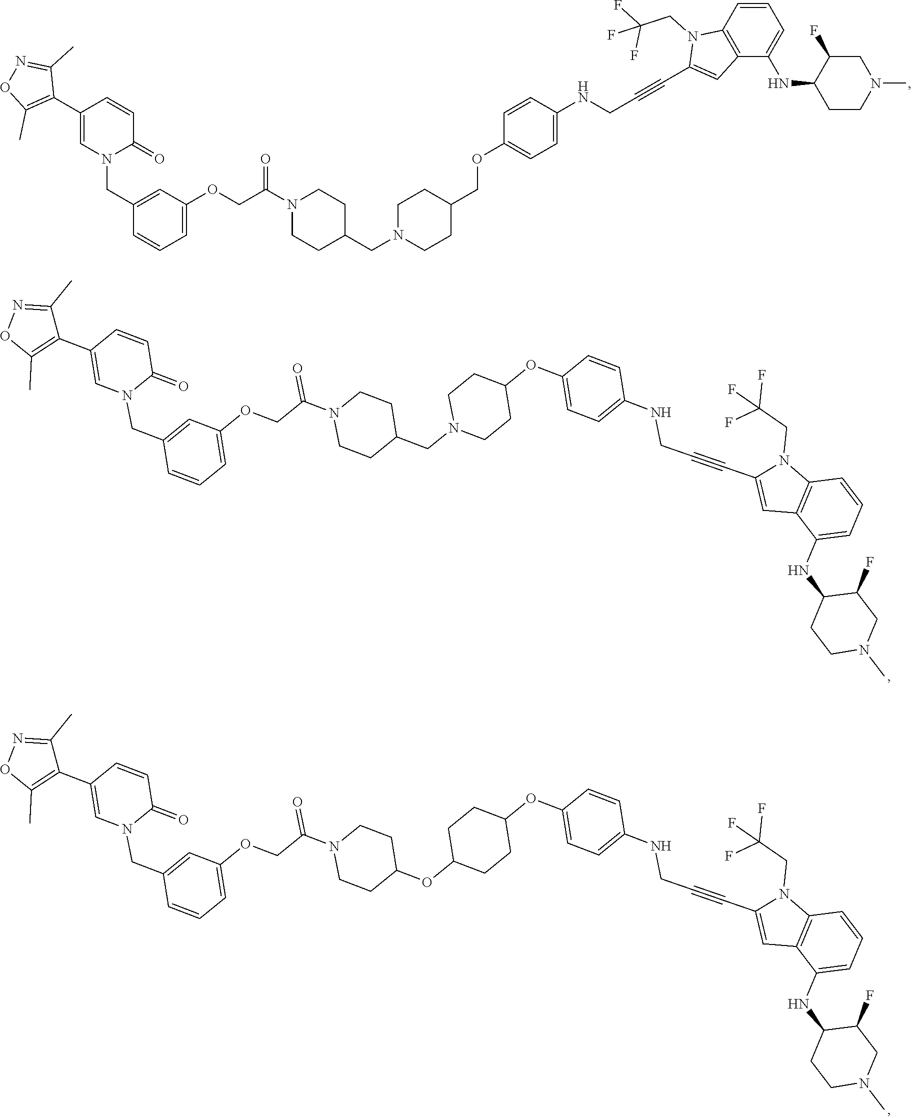 [SMILES: Cc1noc(C)c1-c1ccc(=O)n(Cc2cccc(OCC(=O)N3CCC(CN4CCC(COc5ccc(NCC#Cc6cc7c(N[C@@H]8CCN(C)C[C@@H]8F)cccc7n6CC(F)(F)F)cc5)CC4)CC3)c2)c1 ; Cc1noc(C)c1-c1ccc(=O)n(Cc2cccc(OCC(=O)N3CCC(CN4CCC(Oc5ccc(NCC#Cc6cc7c(N[C@@H]8CCN(C)C[C@@H]8F)cccc7n6CC(F)(F)F)cc5)CC4)CC3)c2)c1 ; Cc1noc(C)c1-c1ccc(=O)n(Cc2cccc(OCC(=O)N3CCC(OC4CCC(Oc5ccc(NCC#Cc6cc7c(N[C@@H]8CCN(C)C[C@@H]8F)cccc7n6CC(F)(F)F)cc5)CC4)CC3)c2)c1]

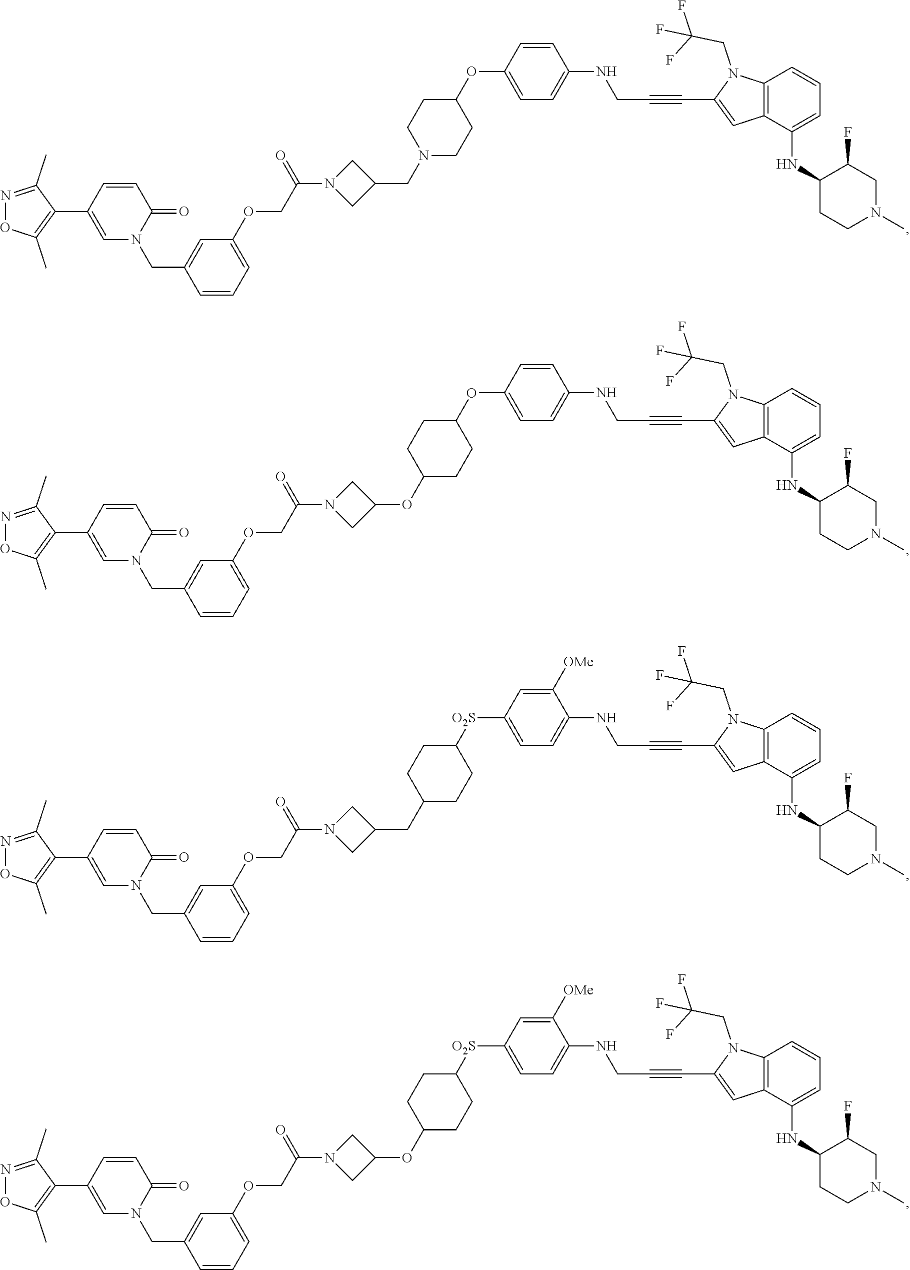

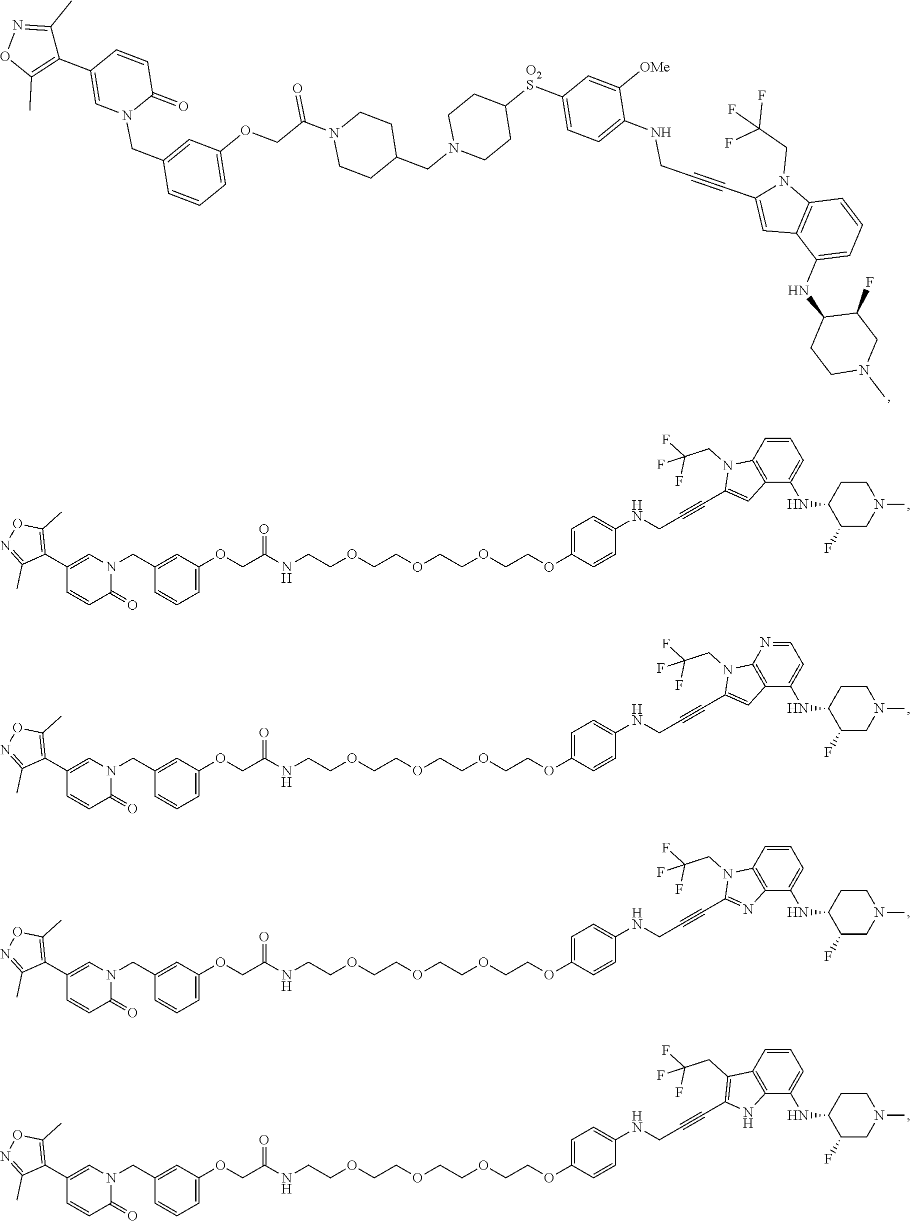

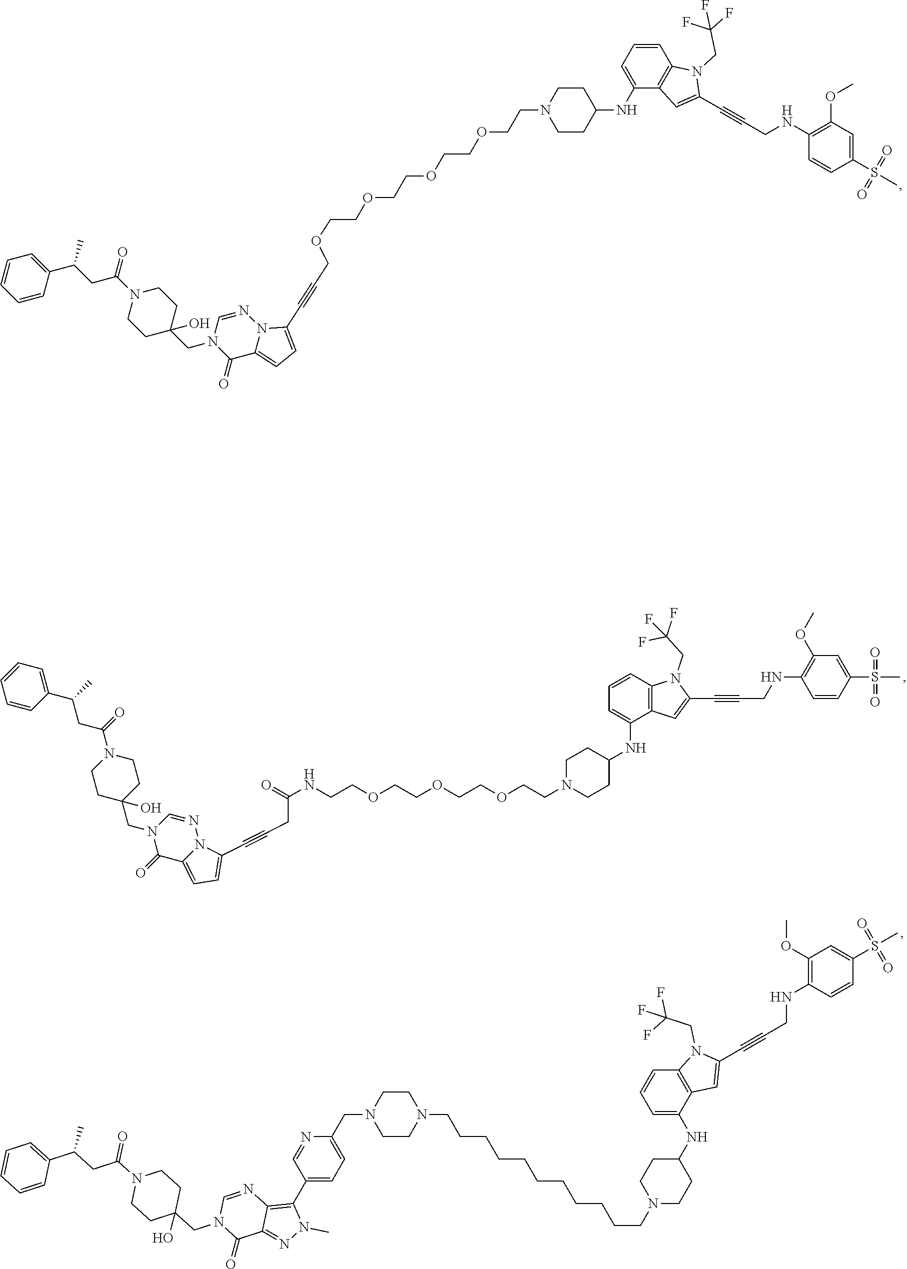 [SMILES: COc1cc(S(C)(=O)=O)ccc1NCC#Cc1cc2c(NC3CCN(CCOCCOCCOCCOCC#Cc4ccc5c(=O)n(CC6(O)CCN(C(=O)C[C@@H](C)c7ccccc7)CC6)cnn45)CC3)cccc2n1CC(F)(F)F ; COc1cc(S(C)(=O)=O)ccc1NCC#Cc1cc2c(NC3CCN(CCOCCOCCOCCNC(=O)CC#Cc4ccc5c(=O)n(CC6(O)CCN(C(=O)C[C@@H](C)c7ccccc7)CC6)cnn45)CC3)cccc2n1CC(F)(F)F ; COc1cc(S(C)(=O)=O)ccc1NCC#Cc1cc2c(NC3CCN(CCCCCCCCCCCN4CCN(Cc5ccc(-c6c7ncn(CC8(O)CCN(C(=O)C[C@@H](C)c9ccccc9)CC8)c(=O)c7nn6C)cn5)CC4)CC3)cccc2n1CC(F)(F)F]

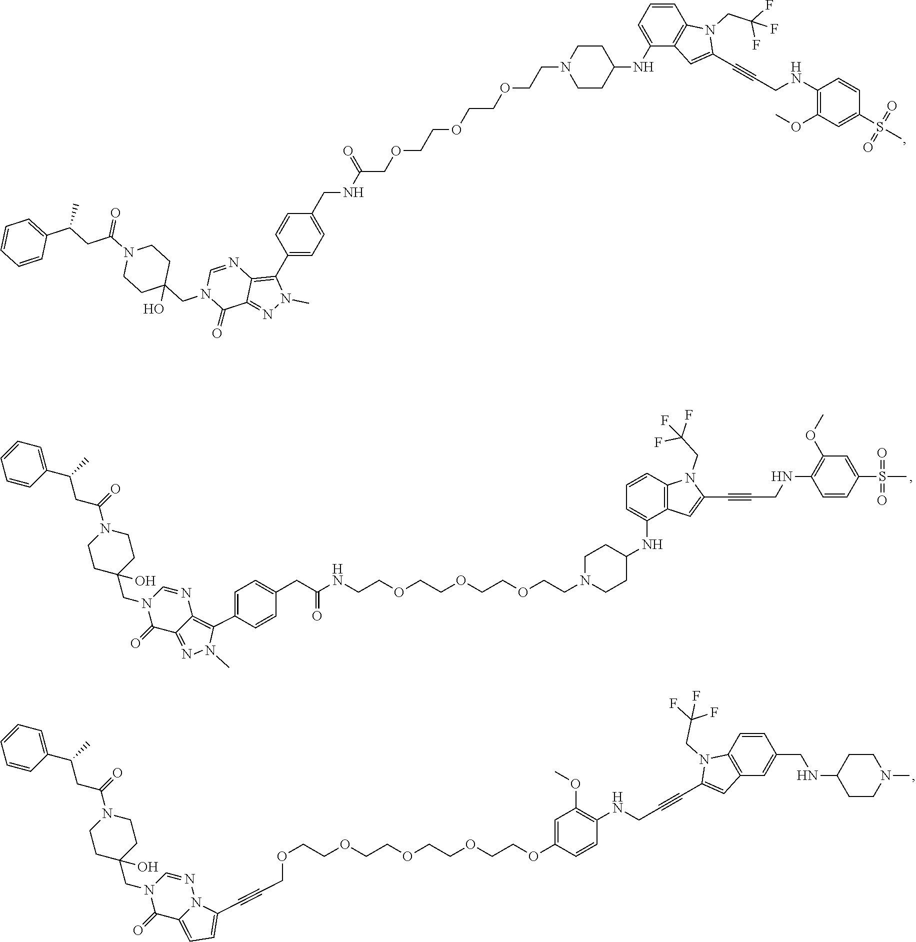 [SMILES: COc1cc(S(C)(=O)=O)ccc1NCC#Cc1cc2c(NC3CCN(CCOCCOCCOCC(=O)NCc4ccc(-c5c6ncn(CC7(O)CCN(C(=O)C[C@@H](C)c8ccccc8)CC7)c(=O)c6nn5C)cc4)CC3)cccc2n1CC(F)(F)F ; COc1cc(S(C)(=O)=O)ccc1NCC#Cc1cc2c(NC3CCN(CCOCCOCCOCCNC(=O)Cc4ccc(-c5c6ncn(CC7(O)CCN(C(=O)C[C@@H](C)c8ccccc8)CC7)c(=O)c6nn5C)cc4)CC3)cccc2n1CC(F)(F)F ; COc1cc(OCCOCCOCCOCCOCC#Cc2ccc3c(=O)n(CC4(O)CCN(C(=O)C[C@@H](C)c5ccccc5)CC4)cnn23)ccc1NCC#Cc1cc2cc(CNC3CCN(C)CC3)ccc2n1CC(F)(F)F]

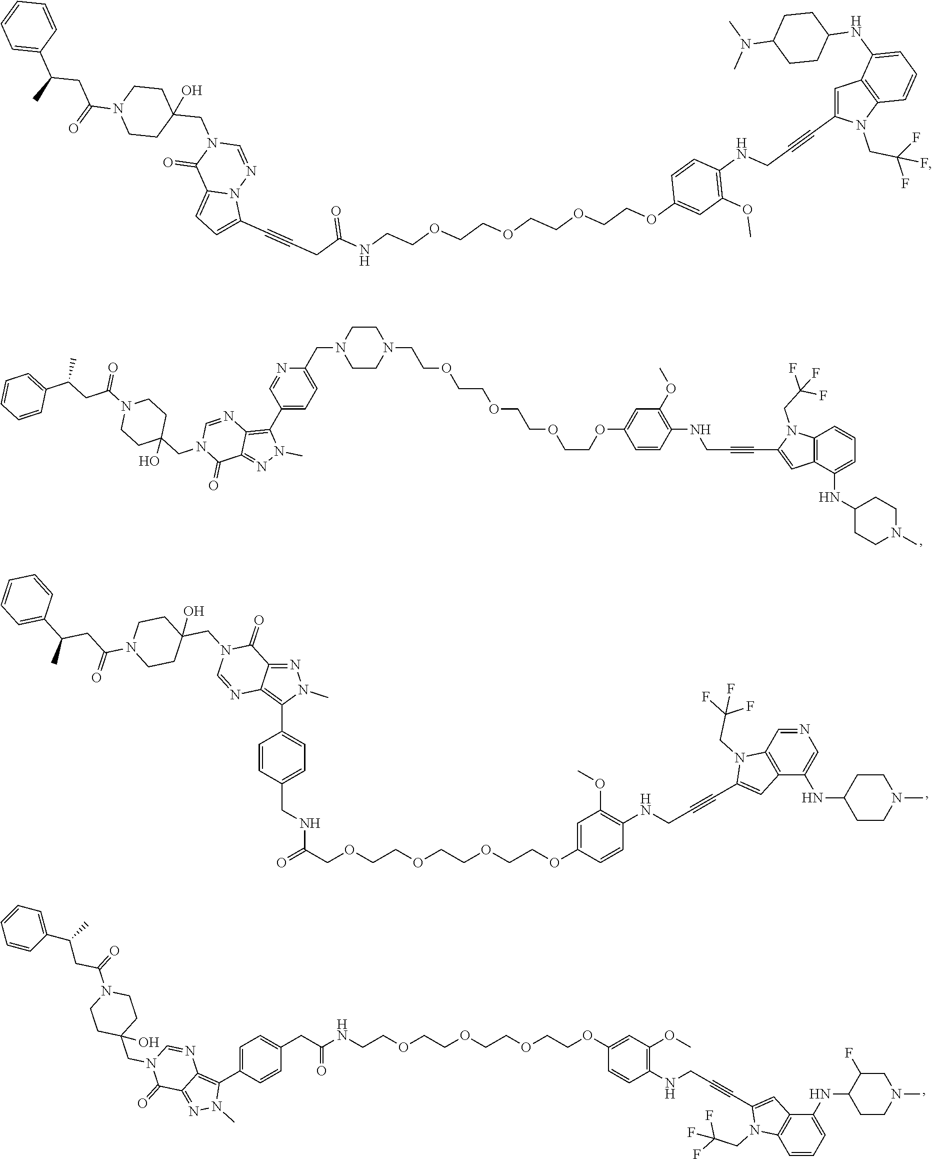

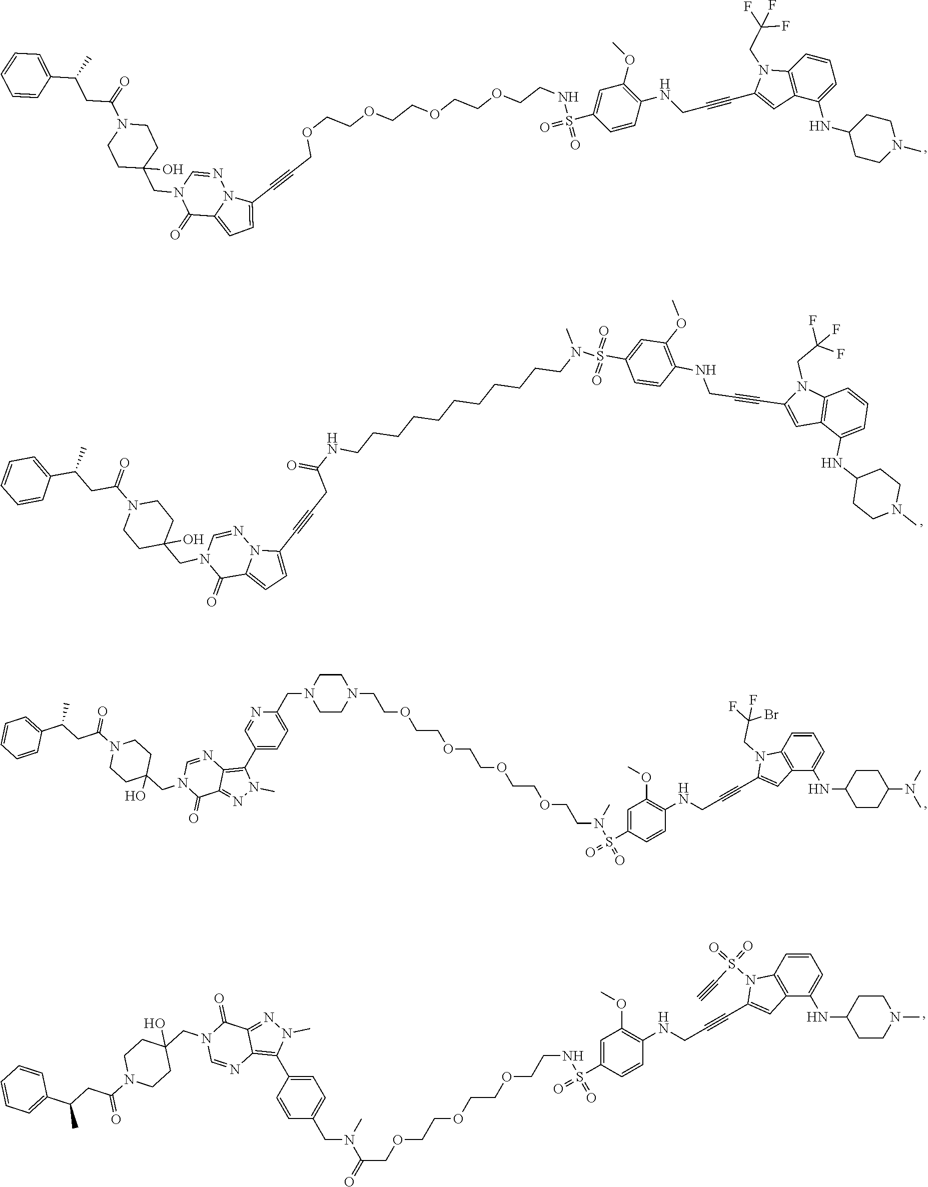

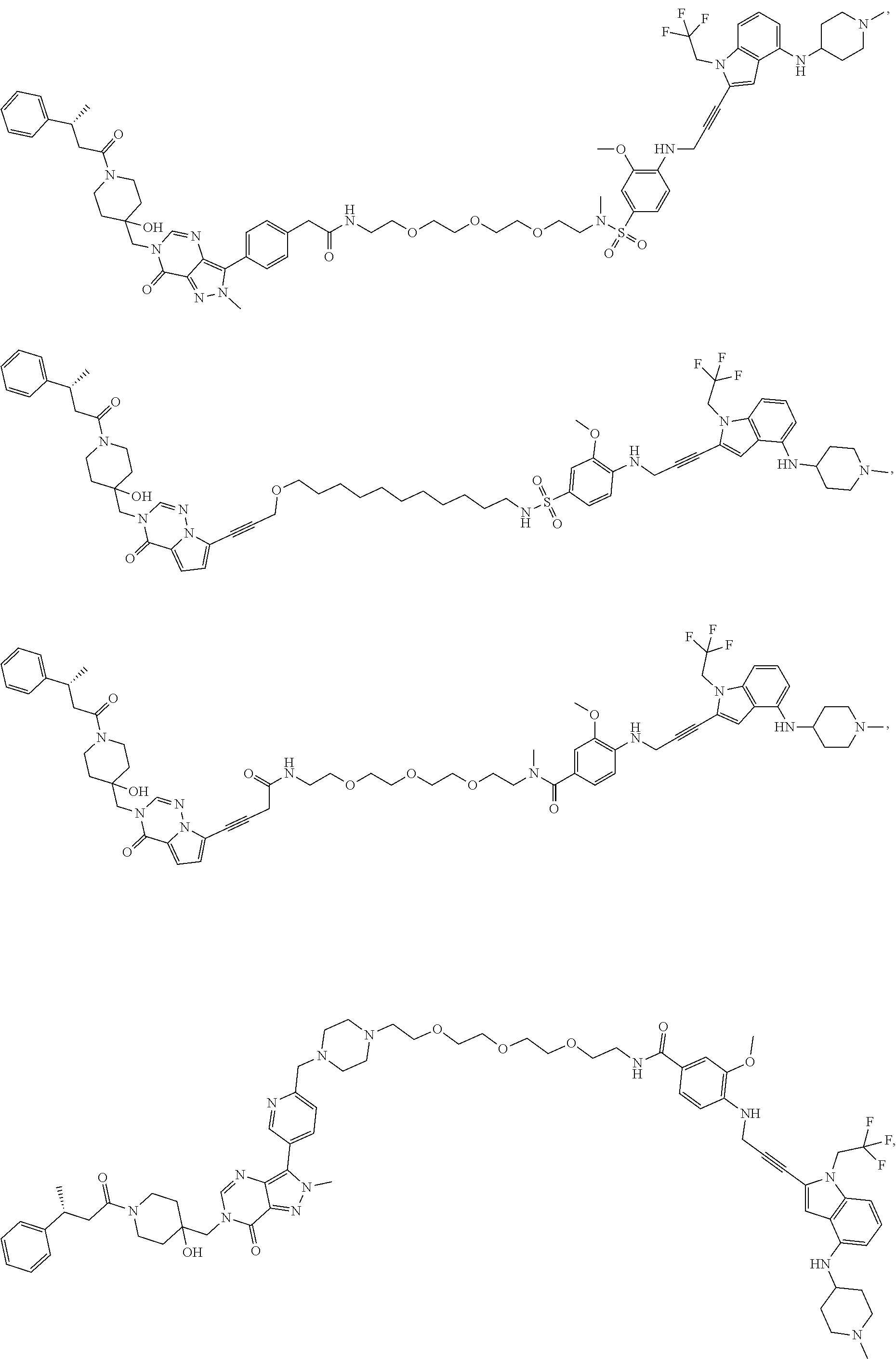

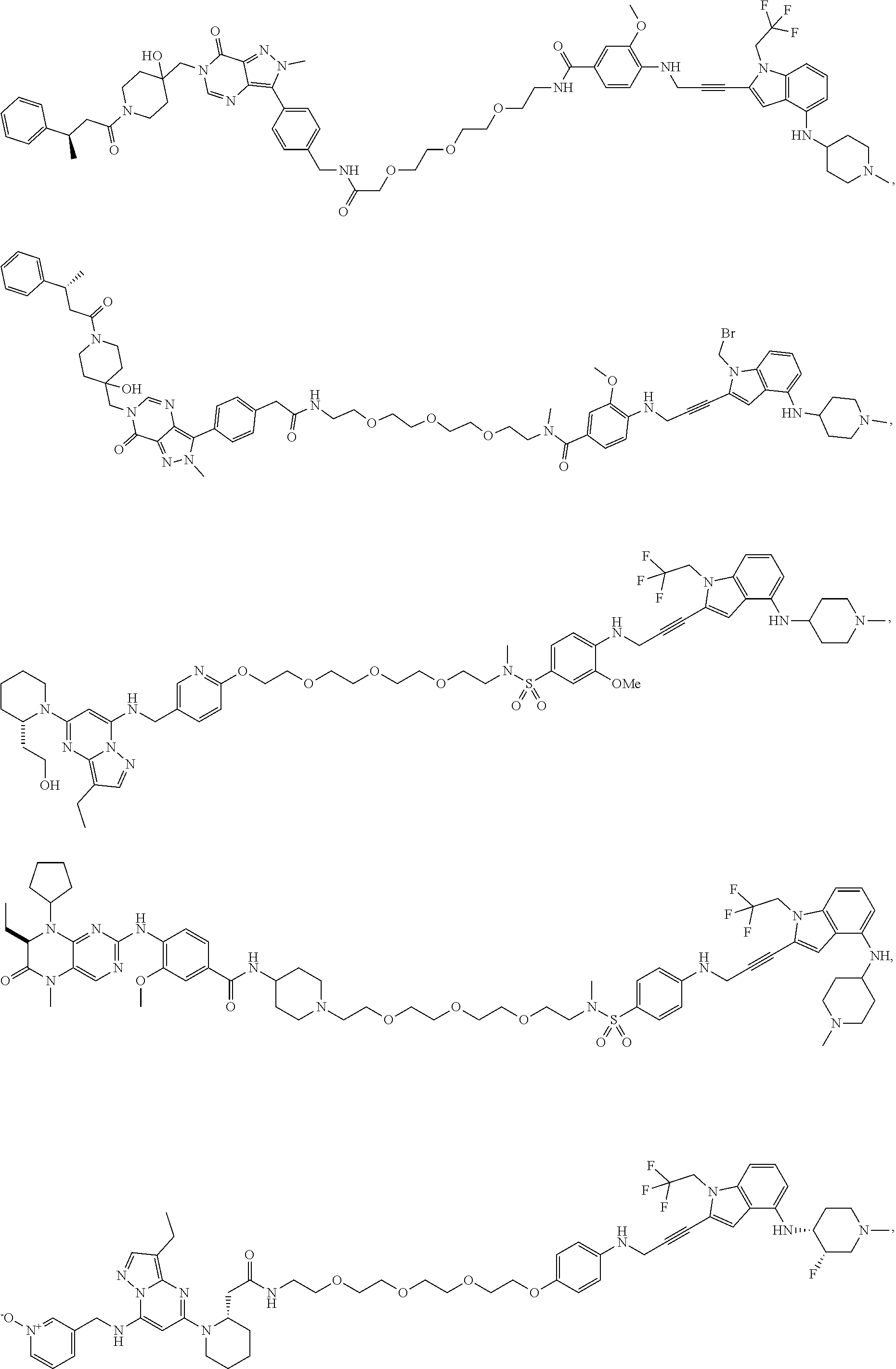

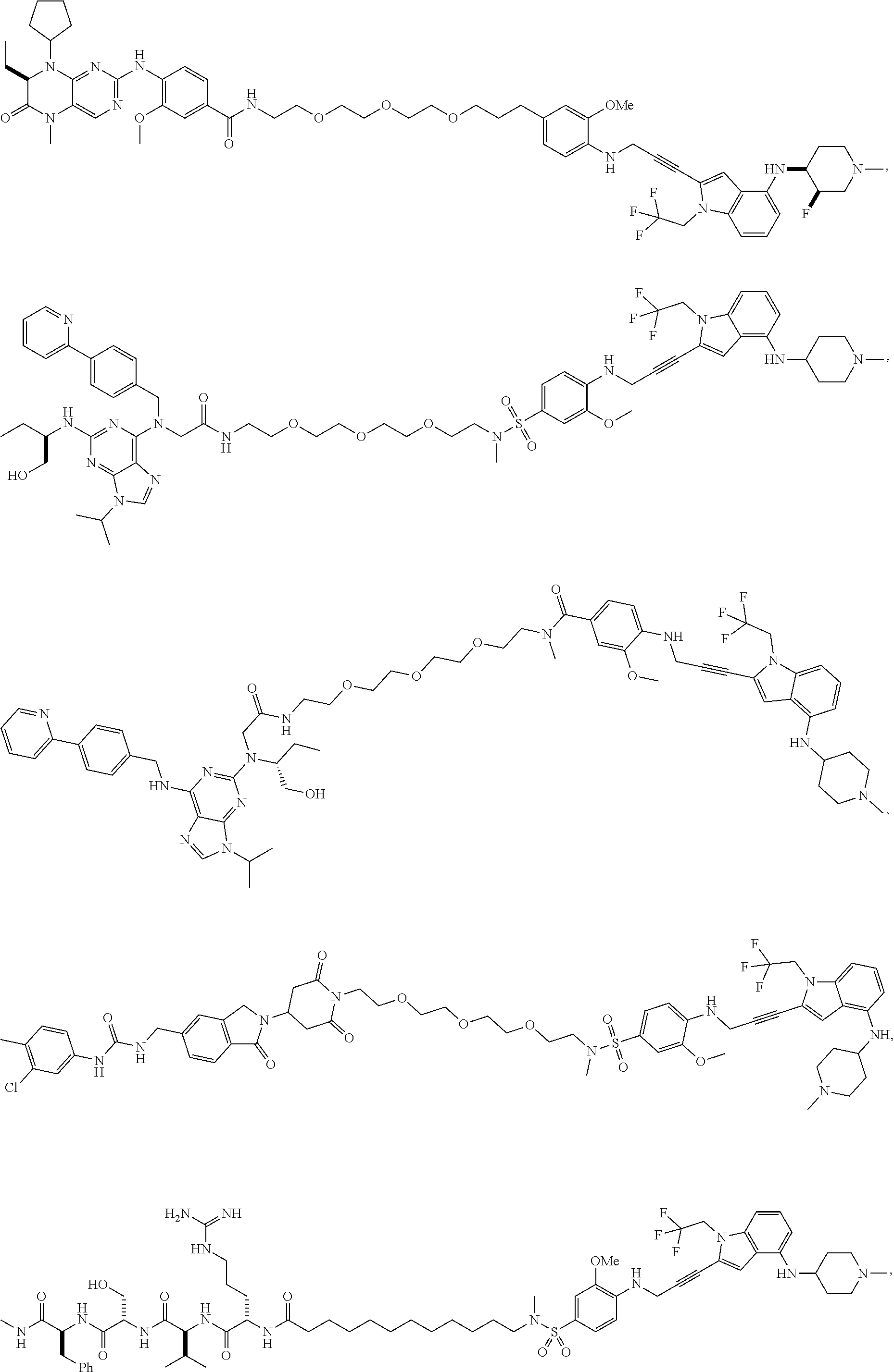

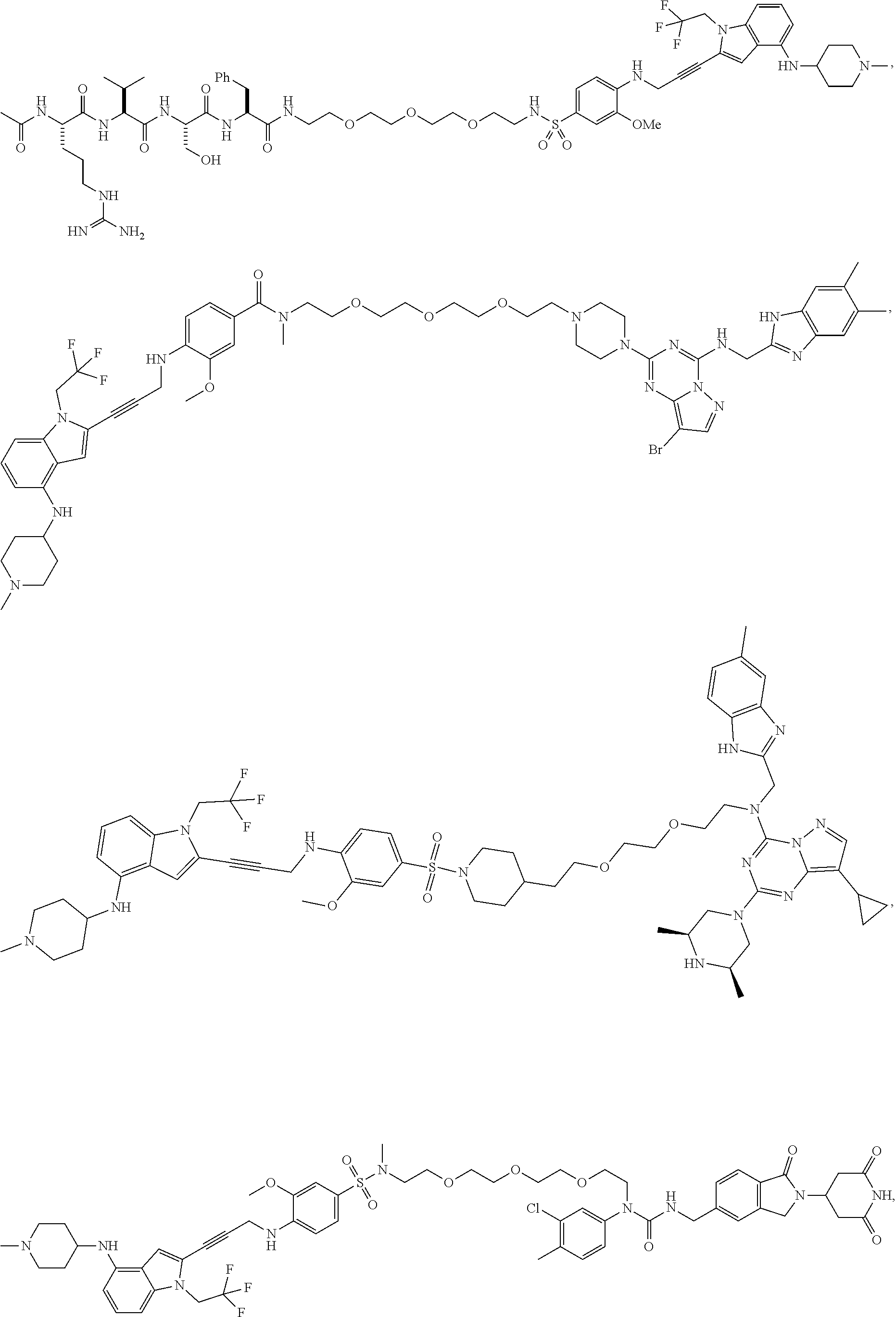

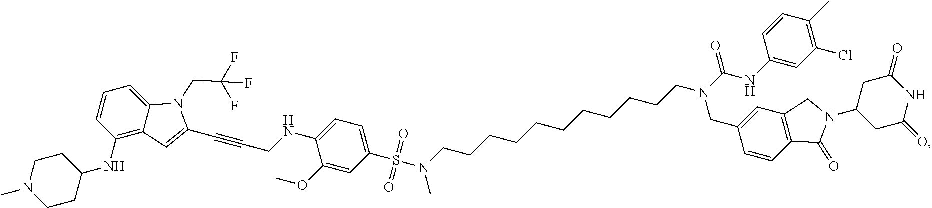 [SMILES: COc1cc(S(=O)(=O)N(C)CCCCCCCCCCCN(Cc2ccc3c(c2)CN(C2CC(=O)NC(=O)C2)C3=O)C(=O)Nc2ccc(C)c(Cl)c2)ccc1NCC#Cc1cc2c(NC3CCN(C)CC3)cccc2n1CC(F)(F)F]

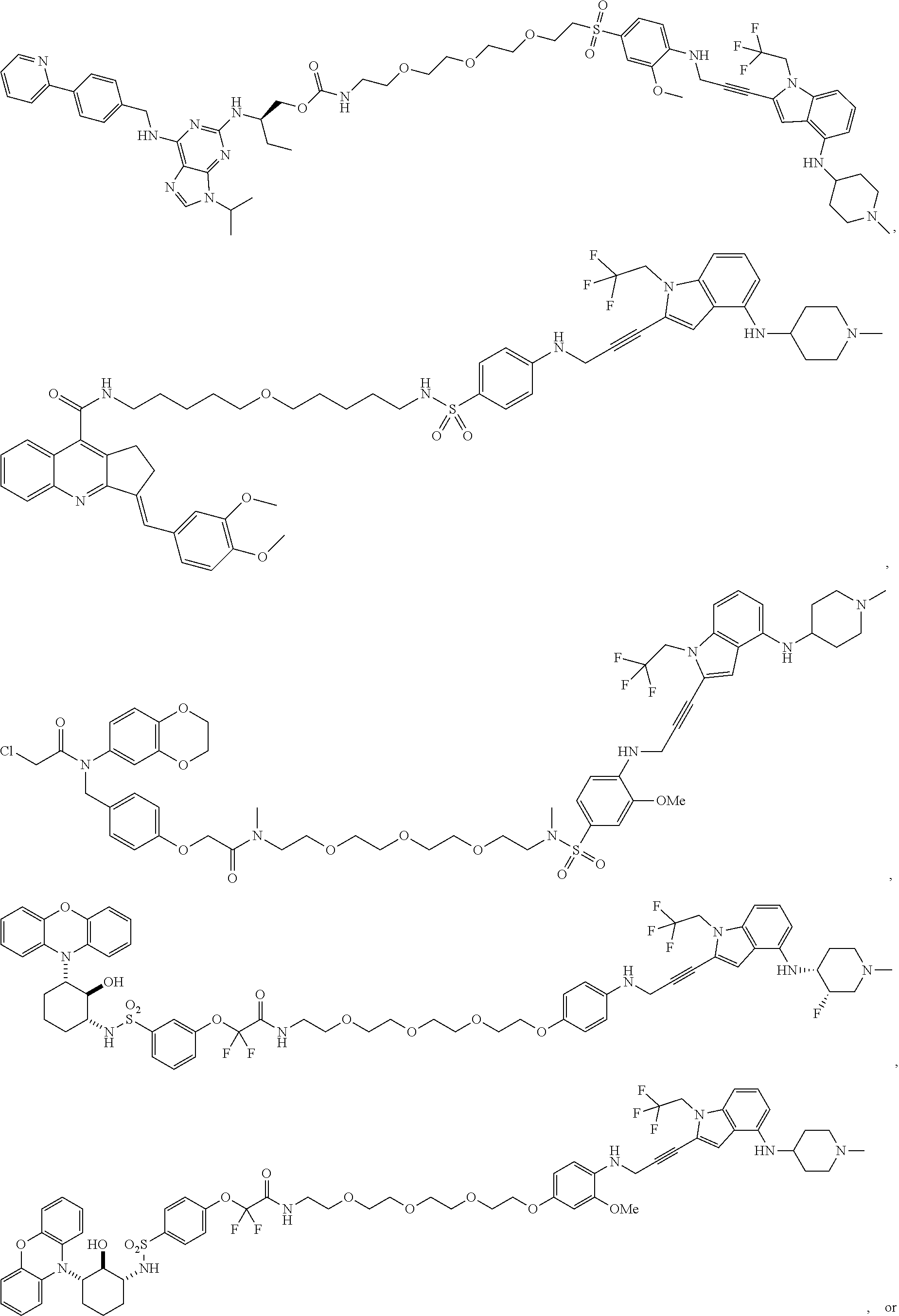

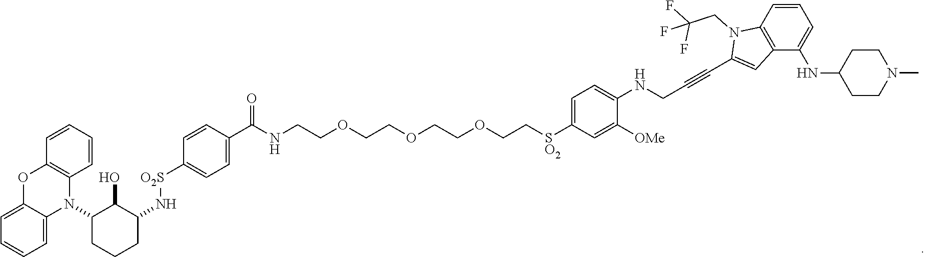

77. (canceled)

78. (canceled)

79. (canceled)

80. (canceled)

81. (canceled)

82. (canceled)


SEQUENCE LISTING (1 sequence)

SEQ ID NO: 1 - protein, 393 aa, Homo sapiens [sequence omitted]